(19) United States
(12) Patent Application Publication
(10) Pub. No.: US 2023/0105376 A1
(43) Pub. Date: Apr. 6, 2023

(54) PIV5-BASED CORONAVIRUS VACCINES AND METHODS OF USE THEREOF

(71) Applicant: CYANVAC LLC, Athens, GA (US)

(72) Inventors: Zhuo LI, Bogart, GA (US); Biao HE, Watkinsville, GA (US); Hong JIN, Cupertino, CA (US); Ashley BEAVIS, Athens, GA (US); Maria Cristina GINGERICH, Athens, GA (US)

(73) Assignee: CYANVAC LLC, Athens, GA (US)

(21) Appl. No.: 17/933,812

(22) Filed: Sep. 20, 2022

Related U.S. Application Data
(60) Provisional application No. 63/246,161, filed on Sep. 20, 2021.
(60) Provisional application No. 63/365,934, filed on Jun. 6, 2022.

(51) Int. Cl. A61K 39/215; C12N 15/86; A61P 31/14

(52) U.S. Cl. CPC A61K 39/215; C12N 15/86; A61P 31/14; C12N 2760/18743; C12N 2760/18734; A61K 2039/545

(57) ABSTRACT

The present invention provides constructs of the parainfluenza virus type-5 (PIV5) virus expressing the SARS-CoV-2 envelope spike (S) and nucleocapsid (N) proteins of SARS-CoV-2 variants for use as safe, stable, efficacious, and cost-effective vaccines against COVID-19.

### REFERENCE TO RELATED APPLICATIONS

This application is the United States non-provisional patent application of U.S. Provisional Application Ser. No. 63/246,161, filed Sep. 20, 2021, and of U.S. Provisional Application Ser. No. 63/365,934, filed Jun. 6, 2022, each of which are hereby incorporated by this reference in their entireties.

### REFERENCE TO SEQUENCE LISTING

The instant application contains a Sequence Listing which has been submitted electronically in ST26 .XML format and is hereby incorporated by reference in its entirety. Said ST26 .XML copy, created on Sep. 20, 2022, is named “065095002US” and is 473 kilobytes in size.

### FIELD OF THE INVENTION

The invention is generally related to the field of immunomodulation, and more particularly to compositions and methods for modulating immune responses in a subject.

### BACKGROUND OF THE INVENTION

SARS-CoV-2 is a novel coronavirus that was first identified in Wuhan, China in December 2019 as a cause of pneumonia and has subsequently spread globally to cause the COVID-19 pandemic. According to the World Health Organization (WHO), since 2019, COVID-19 has spread globally, infected more than 519 million people, and caused at least 6 million deaths. SARS-CoV-2 initially infects the upper respiratory tract epithelium (Wolfel, R., et al., Nature, 581(7809):465 (2020)) but can progress to the lower respiratory tract to cause pneumonia and acute respiratory distress syndrome (ARDS) (Huang, C., et al., Lancet, 395(10223):497 (2020)). Since the beginning of the 2019 coronavirus disease (COVID-19) pandemic, numerous SARS-CoV-2 variants have emerged. WHO defines a SARS-CoV-2 variant of concern (VOC) as a variant that affects virus transmissibility and COVID-19 epidemiology, increases virulence and pathogenicity, or decreases the effectiveness of in-place public health measures. Current VOCs include delta and omicron, while previously circulating VOCs include alpha, beta, and gamma.

The global spread of SARS-CoV-2 prompted rapid development of prophylactic vaccines. Vaccines and antibody therapies have been introduced worldwide providing a ray of hope for the end of the COVID-19 pandemic. Currently, three vaccines are approved for use in the United States. The vaccines developed by Pfizer and Moderna are based on mRNA technology, while the vaccine produced by Johnson & Johnson (J&J) utilizes a human adenovirus type 26 vector. A vaccine produced by AstraZeneca employs a Chimpanzee adenovirus vector and is approved for use in the European Union (Ura, T., et al., Vaccine, 39(2): 197 (2021)). Since May 2022, 11 billion vaccine doses have been administered worldwide. However, SARS-CoV-2 variants have demonstrated immune escape in previously infected and fully vaccinated individuals. Compared to neutralization of alpha variant, serum from convalescent individuals was four-fold less effective against delta variant. The same study found that serum from individuals who received two doses of Pfizer's vaccine has delta-neutralizing abilities three- to five-fold lower than its alpha-neutralizing abilities. This immune escape was correlated to epitopes found on the SARS-CoV-2 spike protein (Planas, D., et al., Nature, 596(7871): 276 (2021)). Another study found that the omicron-neutralizing ability of serum from WA1-convalescent individuals is eight-fold lower than its WA1-neutralizing ability (Zhang, L., et al., Emerg Microbes Infect, 11(1): 1 (2022)). Analysis of published reports indicates that for individuals vaccinated with Pfizer, Moderna, J&J, or AstraZeneca vaccines, vaccine efficacy decreased by approximately 21 percentage points within one to six months after full vaccination. This decrease in efficacy was associated with waning immunity (Feikin, D. R., et al., Lancet, 399(10328): 924 (2022)).

Therefore, compounding waning immunity with the emergence of variants capable of immune escape, there is an urgent need for novel SARS-CoV-2 vaccine candidates with long-lasting immunity and protective against variants.

### SUMMARY OF THE INVENTION

The present disclosure presents an intranasal viral-vectored vaccine against SARS-CoV-2 variants. The disclosure includes a viral expression vector having a parainfluenza virus 5 (PIV5) genome having a heterologous nucleic acid sequence with at least 98% sequence identity to SEQ ID NOs: 61, 62, 63, 64, 65, 66, 67, 68, 69, 70, 71, 72, 73, 74, 75, 76, 77, 78, 79, or 80, wherein the viral expression vector expresses a heterologous polypeptide comprising a coronavirus spike (S) and/or nucleocapsid (N) proteins.

In some aspects of the viral expression vector, the coronavirus S protein includes the coronavirus S protein of the severe acute respiratory syndrome coronavirus 2 (SARS-CoV-2) and the coronavirus N protein is the coronavirus N protein of SARS-CoV-2, a variant of interest or a variant of concern of SARS-CoV-2.

In some aspects of the viral expression vector, the coronavirus S protein is the coronavirus S protein of a SARS-CoV-2 beta variant, a SARS-CoV-2 gamma variant, a SARS-CoV-2 delta variant, or a SARS-CoV-2 omicron variant and the coronavirus N protein is the coronavirus N protein of a SARS-CoV-2 beta variant, a SARS-CoV-2 gamma variant, a SARS-CoV-2 delta variant, or a SARS-CoV-2 omicron variant.

In some aspects of the viral expression vector, the coronavirus S protein comprises the coronavirus S protein of SARS-CoV-2 and wherein the cytoplasmic tail of the coronavirus S protein has been replaced with the cytoplasmic tail of the fusion (F) protein of PINS.

In some aspects of the viral expression vector, the coronavirus S protein of a variant of SARS-CoV-2 has been inserted between the PIV5 small hydrophobic (SH) and hemagglutinin (HN) genes and the coronavirus N protein of a variant of SARS-CoV-2 has been inserted between the PIV5 HN and polymerase (L) genes.

In some aspects of the viral expression vector, the coronavirus S protein of a variant of SARS-CoV-2 has been inserted between the PIV5 HN and polymerase (L) genes.

In some aspects of the viral expression vector, the coronavirus S protein is a S protein from a variant of interest or a variant of concern of SARS-CoV-2 and the coronavirus N protein is a N protein from different variant of interest or a variant of concern of SARS-CoV-2.

In some aspects of the viral expression vector, the PIV5 small hydrophobic (SH) gene has been replaced with the coronavirus N protein of SARS-CoV-2.

In some aspects of the viral expression vector, the PIV5 genome further comprises one or more mutations comprising a mutation of the V/P gene, a mutation of the shared N-terminus of the V and P proteins, a mutation of residues 26, 32, 33, 50, 102, and/or 157 of the shared N-terminus of the V and P proteins, a mutation lacking the C-terminus of the V protein, a mutation lacking the small hydrophobic (SH) protein, a mutation of the fusion (F) protein, a mutation of the phosphoprotein (P), a mutation of the large RNA polymerase (L) protein, a mutation incorporating residues from canine parainfluenza virus, a mutation inducing apoptosis, or a combination thereof.

In some aspects of the viral expression vector, one or more mutations comprise PIV5VΔC, PIV5ΔSH, PIV5-P-S308G, or a combination thereof.

In some aspects of the viral expression vector, the heterologous polypeptide comprises a CPI V/P gene that contains mutations at amino acid residue S157 or S308, or the combination thereof, wherein serine (S) is substituted with an amino acid residue selected from a group consisting of alanine (A), asparagine (B), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), selenocysteine (U), valine (V), tryptophan (W), tyrosine (Y), and glutamine (Z).

In some aspects of the viral expression vector, the amino acid substitution at amino acid residue S157 comprises a substitution of serine (S) to phenylalanine (F) and the amino acid substitution at amino acid residue S308 comprises a substitution of serine (S) to alanine (A) or Glycine (G).

In some aspects of the viral expression vector, a viral particle comprises the viral expression vector.

The present disclosure also presents a composition comprising the viral expression vector presented above, a viral particle presented above, or a combination thereof.

In some aspects of the composition, a heterologous coronavirus spike (S) and nucleocapsid (N) proteins are expressed in a cell by contacting the cell with the composition.

The present disclosure also presents a method of inducing an immune response in a subject having coronavirus disease 2019 (COVID-19) to coronavirus spike (S) and nucleocapsid (N) proteins, the method comprising administering the composition presented above to the subject, wherein the immune response comprises a humoral immune response and/or a cellular immune response.

In some aspects of the method, the subject is vaccinated against COVID-19), the method comprising administering the composition to the subject.

In some aspects of the method, the composition is administered intranasally, intramuscularly, topically, or orally.

The present disclosure also presents a method of inducing in a subject an immune response comprising administering a PIV-5 booster vaccine composition comprising a viral expression vector or a viral particle having a PIV5 genome comprising a heterologous nucleic acid sequence with at least 98% sequence identity to SEQ ID NOs: 61, 62, 63, 64, 65, 66, 67, 68, 69, 70, 71, 72, 73, 74, 75, 76, 77, 78, 79, or 80, wherein said subject has previously received a primary vaccination against SARS-COV-2.

### BRIEF DESCRIPTION OF THE DRAWINGS

FIG. 1 shows a schematic of the vaccine virus indicating the location of SARS-CoV-2 genes and their corresponding variant of origin.

FIG. 2 shows an outline of hamster immunization and challenge.

FIGS. 3A-3B show hamster study 1. Following challenge with SARS-CoV-2 WA1 (FIG. 3A) or CA1/alpha (FIG. 3B), the hamster weights were monitored. Weights are graphed as percent of weight on the day of immunization, or 0 dpi.

FIGS. 4A-4B show infectious SARS-CoV-2 in hamster lungs post-WA1 (FIG. 4A) or CA1/alpha (FIG. 4B) challenge. 5 dpc, lungs were harvested and lung homogenate was plagued on Vero cells. The limit of detection (LOD) is indicated by the dotted line.

FIGS. 5A-5D show SARS-CoV-2 RNA in hamster lungs post-WA1 (FIGS. 5A-5B) or CA1/alpha (FIGS. 5C-5D) challenge. CT values for each sample are plotted. The standard curves for challenge variant were used to calculate the CT value indicative of PCR-negative and 1 PFU/reaction (rxn).

FIG. 6 is a schematic of the study showing the efficacy of CVXGA1 and CVXGA3 in African green monkeys.

FIGS. 7A-7B show SARS-CoV-2 S-specific cellular immune response in immunized African green monkeys (AGMs). Prior vaccine dosing at day −1, and 7, 14, and 28 dpi, PBMCs were isolated from immunized AGMs. The PBMCs were stimulated with SARS-CoV-2 S peptide, and the number of IFN-γ spots were quantified. Graphed are the number of spots per 200,000 PBMCs.

FIG. 8 shows Anti-SARS-CoV-2 S IgG antibody response in immunized AGMs. Prior dosing at day −1 and 28 dpi, serum was collected from immunized AGMs, serially diluted and incubated on T96 plates coated with 1 μg/mL purified SARS-CoV-2 S protein. The wells were washed and incubated with anti-monkey IgG antibody conjugated to HRP. The wells were washed and incubated with KPL SureBlue RMB substrate. The antibody titer was calculated as log 10 of the highest serum dilution at which the OD450 was greater than two standard deviations above the mean OD450 of naïve serum. The dotted line indicates the limit of detection (LOD).

FIGS. 9A-9B show infectious SARS-CoV-2 challenge virus titer in nasal wash (FIG. 9A) and BAL (FIG. 9B). NW and BAL were collected at 2, 4, and 7 dpc. The samples were serially diluted and incubated on Vero cells. 1 hr post-infection, a methylcellulose overlay was added onto the cells. Three days post-infection, the cells were fixed and stained, and the plaques were counted.

FIGS. 10A-10B shows SARS-CoV-2 challenge virus RNA load in nasal wash (FIG. 10A) and BAL (FIG. 10B) qRT-PCR. 2, 4, and 7 dpc, NW and BAL were collected. The samples were mixed 1:10 in Trizol, and RNA was extracted. qRT-PCR was performed on the RNA samples. To calculate the PCR negative and theoretical 1 PFU/rxn values, a standard curve was generated with RNA extracted from SARS-CoV-2 alpha virus.

FIG. 11 is a schematic of the PIV5 and CVXGA vaccines. The PIV5 genome has 7 genes 3′ to 5′: NP, V/P, M, F, SH, HN, L. The S genes from SARS-CoV-2 WA1 (CVXGA1), beta variant (CVXGA3), gamma variant (CVXGA5), delta variant (CVXGA13), and omicron variant (CVXGA14) had their cytoplasmic tails replaced with the PIV5 F cytoplasmic tail and inserted between PIV5 genes SH and HN. CVXGA2 also has SARS-CoV-2 WA1 N between PIV5 HN and L genes.

FIGS. 12A-12AA show CVXGA vaccine antigen expression panels. Vero cells were infected at MOI 0.01 and fixed with 80% methanol at 3 days post-infection. FIGS. 12A-12S show intracellular expression of PIV5-V/P, SARS-CoV-2-S, and SARS-CoV-2-N. The cells were incubated with anti-PIV5 V/P, -SARS-CoV-2 S, or SARS-CoV-2 N antibodies, followed by Cy3-conjugated secondary antibody. The cells were washed with PBS and imaged at 10× with an EVOS M5000 microscope (Thermo Fisher Scientific). FIGS. 12T-12AA show cell-to-cell fusion induced by SARS-CoV-2 S expression. Infected cells were imaged at 10× with an Evos M5000 microscope. Arrows indicate syncytium, multinucleated cells.

FIGS. 13A-13B show that immunization of hamsters with CVXGA1, CVXGA2, CVXGA3, and CVXGA5 induces anti-SARS-CoV-2 S IgG antibodies. FIG. 13A shows intracellular expression of PIV5-V/P, SARS-CoV-2-S, and SARS-CoV-2-N. The cells were incubated with anti-PIV5 V/P, -SARS-CoV-2 S, or SARS-CoV-2 N antibodies, followed by Cy3-conjugated secondary antibody. The cells were washed with PBS and imaged at 10× with an EVOS M5000 microscope (Thermo Fisher Scientific). FIG. 13B shows cell-to-cell fusion induced by SARS-CoV-2 S expression. Infected cells were imaged at 10× with an Evos M5000 microscope. Arrows indicate syncytium, multinucleated cells.

FIGS. 14A-14F show immunization with CVXGA1 protects hamsters from challenge with SARS-CoV-2 WA1 and alpha variant. Following challenge with WA1 (FIG. 14A) or alpha variant (FIG. 14B), hamster weights were monitored daily for five days and graphed as percent day 0 weight. Statistical significance was calculated for each timepoint between each group and PIV5-immunized hamsters with t tests (* p<0.05, ** p<0.01, *** p<0.001, **** p<0.0001). 5 dpc with WA1 (FIG. 14C) or alpha variant (FIG. 14D), the hamster lungs were harvested and homogenized in 1.7 mL PBS. Viral load was quantified via plaque assay in Vero E6 cells and graphed as PFU/mL lung homogenate. The limit of detection (LOD) is indicated by the dotted line. Error bars represent the standard error of the means. RNA was extracted from lung homogenate and SARS-CoV-2 WA1 (FIG. 14E) or alpha variant (FIG. 14F) vRNA was quantified via RT-qPCR. The cycle threshold (Ct) value for each sample is presented and error bars represent the standard error of the means. The known viral titers of WA1 and alpha variant were used to generate standard curves for FIG. 14E and FIG. 14F, respectively, and calculate the Ct values equating to 1 PFU per reaction (rxn). The Ct value generated from RNA extracted from sterile water is denoted by a dotted line labelled PCR negative. The limit of detection (LOD) is indicated by a dotted line at Ct value=40, the number of PCR cycles. Comparing each group to PIV5-immunized hamsters, statistical significance was calculated with one-way ANOVA (** p<0.01, *** p<0.001).

FIGS. 15A-15F show that immunization with CVXGA1 protects hamsters from challenge with SARS-CoV-2 delta variant. FIG. 15A is an immunization schematic. Golden Syrian hamsters (n=5) were intranasally immunized with 100 μL PBS, 3×10⁵ PFU CVXGA1, 2×10⁵ PFU CVXGA3, or 1.5×10⁵ PFU CVXGA13. Blood was collected at 19 dpi. At 28 dpi, the hamsters were challenged with 10⁴ PFU of SARS-CoV-2 delta variant. Following challenge infection, the hamster weights were monitored daily before terminating the study and harvesting lungs at 5 dpc. 19 dpc, anti-SARS-CoV-2 WA1 S (FIG. 15B) and RBD (FIG. 15C) IgG antibodies were quantified via ELISA. Antibody titer was calculated as log₁₀ of the highest serum dilution at which the OD₄₅₀ was greater than two standard deviations above the mean OD₄₅₀ of naïve serum. The limit of detection (LOD) is indicated by the dotted line. Error bars represent the standard error of the means. Comparing each group to the vector control, statistical significance was calculated with one-way ANOVA (** p<0.01, **** p<0.0001). FIG. 15D shows the results following challenge. Hamster weights were monitored daily for five days and graphed as percent day 0 weight. Statistical significance was calculated for each timepoint between each group and PIV5-immunized hamsters with t tests (* p<0.05, ** p<0.01, *** p<0.001). 5 dpc, the hamster lungs were harvested and homogenized in 1.7 mL PBS. FIG. 15E shows viral load which was quantified via plaque assay in Vero TEMPRSS cells and graphed as PFU/mL lung homogenate. The limit of detection (LOD) is indicated by the dotted line. Error bars represent the standard error of the means. FIG. 15F shows RNA was extracted from lung homogenate and SARS-CoV-2 delta vRNA was quantified via RT-qPCR. The cycle threshold (Ct) value for each sample is presented and error bars represent the standard error of the means. The known viral titer of delta variant was used to generate a standard curves and calculate the Ct value equating to 1 PFU per reaction (rxn). The limit of detection (LOD), PCR negative, is indicated by a dotted line at Ct value=40, the number of PCR cycles.

FIGS. 16A-16H show that immunization with a Covid-19 mRNA vaccine, one dose of CVXGA1 or two doses of CVXGA1, protects hamsters against challenge with delta variant. FIG. 16A is a schematic of immunization. Golden Syrian hamsters received 100 μL PBS intranasally (n=5, Group 1), 2 μg mRNA COVID vaccine (n=25, Group 2), or 100 μL 7×10⁴ PFU CVXGA1 (n=10, Groups 3 & 4). At 29 dpi, hamsters that received the mRNA vaccine were boosted with the mRNA vaccine again and group 3 hamsters received another dose of CVXGA1. At 91 dpi following initial immunization, hamsters who received two doses of mRNA received 2 μg mRNA vaccine (n=5, Group 2A), 100 μL 7×10⁴ PFU CVXGA1 (n=5, Group 2B), 100 μL 10³ PFU CVXGA13 (n=5, Group 2C), 100 μL PBS i.n. (n=5, Group 2D), or 100 μL 10⁴ PFU CVXGA14 (n=5, Group 2E). Blood was collected at 36 and 108 dpi. At 116 dpi, the hamsters were challenged with 10⁵ PFU SARS-CoV-2 delta variant. Following challenge infection, hamster weights were monitored for 5 days and the hamster lungs were harvested. FIG. 16B shows 36 and 108 days post-immunization, anti-SARS-CoV-2 WA1 S IgG antibodies were quantified via ELISA. Antibody titer was calculated as log₁₀ of the highest serum dilution at which the OD₄₅₀ was greater than two standard deviations above the mean OD₄₅₀ of naïve serum. The limit of detection (LOD) is indicated by the dotted line. Error bars represent the standard error of the means. FIGS. 16C-16E show graphs of microneutralization assays performed. Hamster serum was heat-inactivated and serially diluted. The serum was mixed 1:1 with 6×10³ focus-forming units (FFU)/mL SARS-CoV-2 WA1, delta, or omicron and incubated on Vero cells for WA1 or Vero TEMPRSS2 cells for delta and omicron. After 24 hr, the number of infected cells were quantified via immunostain. Neutralization titers were calculated as log₁₀ of the highest serum dilution at which the virus infectivity was reduced by at least 50%. The limit of detection (LOD) is indicated by the dotted line. FIG. 16F shows that following challenge, hamster weights were monitored daily for five days and graphed as percent day 0 weight. Statistical significance was calculated for each timepoint between each group and PBS-immunized hamsters with t tests (* p≤0.05). 5 dpc, the hamster lungs were harvested and homogenized in 1.7 mL PBS. FIG. 16G shows a graph where viral load was quantified via plaque assay in Vero TEMPRSS cells and graphed as PFU/mL lung homogenate. The limit of detection (LOD) is indicated by the dotted line. Error bars represent the standard error of the means. FIG. 16H shows a graph where RNA was extracted from lung homogenate and SARS-CoV-2 delta vRNA was quantified via RT-qPCR. The cycle threshold (Ct) value for each sample is presented and error bars represent the standard error of the means. The known viral titer of delta variant was used to generate a standard curve and calculate the Ct value equating to 1 PFU per reaction (rxn). The Ct value generated from RNA extracted from sterile water is denoted by a dotted line labelled PCR negative. The limit of detection (LOD) is indicated by a dotted line at Ct value=40, the number of PCR cycles. Error bars represent the standard error of the means. Statistical significance was calculated with one-way ANOVA (** p<0.01, **** p<0.0001).

FIGS. 17A-17E are graphs showing that, following two doses of a Covid-19 mRNA vaccine, CVXGA1 boosts the humoral immune response and elicits protection of hamsters against challenge with delta variant. FIG. 17A is a graph showing, anti-SARS-CoV-2 S IgG antibodies 17 days post-boost which were quantified via ELISA. Antibody titer was calculated as log₁₀ of the highest serum dilution at which the OD₄₅₀ was greater than two standard deviations above the mean OD₄₅₀ of naïve serum. The limit of detection (LOD) is indicated by the dotted line. Error bars represent the standard error of the means. Statistical significance was calculated with one-way ANOVA (**** p<0.0001). FIG. 17 B shows microneutralization assays were performed with post-boost sera. Hamster serum was heat-inactivated and serially diluted. The serum was mixed 1:1 with 6×10³ focus-forming units (FFU)/mL SARS-CoV-2 WA1, delta, or omicron and incubated on Vero cells for WA1 or Vero TEMPRSS2 cells for delta and omicron. After 24 hr, the number of infected cells were quantified via immunostain. Neutralization titers were calculated as log₁₀ of the highest serum dilution at which the virus infectivity was reduced by at least 50%. The limit of detection (LOD) is indicated by the dotted line. Statistical significance was calculated between each group and the PBS group with one-way ANOVA (* p≤0.05, ** p<0.01, *** p<0.001, **** p<0.0001). FIG. 17C is a line graph showing that, following challenge, hamster weights were monitored daily for five days and graphed as percent day 0 weight. Statistical significance was calculated for each timepoint between each group and PBS-immunized hamsters with t tests (* p≤0.05, ** p<0.01). 5 dpc, the hamster lungs were harvested and homogenized in 1.7 mL PBS. FIG. 17D shows that no detectable infectious virus in their lungs were observed in boosted hamsters. FIG. 17E shows viral load quantified via plaque assay in Vero TEMPRSS cells and graphed as PFU/mL of lung homogenate. The limit of detection (LOD) is indicated by the dotted line. Error bars represent the standard error of the means. FIG. 17F shows RNA was extracted from lung homogenate and SARS-CoV-2 delta vRNA was quantified via RT-qPCR. The cycle threshold (Ct) value for each sample is presented and error bars represent the standard error of the means. The known viral titer of delta variant was used to generate a standard curve and calculate the Ct value equating to 1 PFU per reaction (rxn). The Ct value generated from RNA extracted from sterile water is denoted by a dotted line labelled PCR negative. The limit of detection (LOD) is indicated by a dotted line at Ct value=40, the number of PCR cycles. Error bars represent the standard error of the means. Statistical significance was calculated with one-way ANOVA (** p<0.01, **** p<0.0001).

FIGS. 18A-18C show RT-qPCR standard curves. RNA was extracted from SARS-CoV-2 WA1 (FIG. 18A), alpha variant (FIG. 18B), and delta variant (FIG. 18C) viral stocks of known titer. The RNA was serially diluted and vRNA was quantified via RT-qPCR. To generate a standard curve, the Ct value was plotted on the y-axis and the PFU per reaction (rxn) was plotted on the x-axis. Dotted lines indicate the Ct value which corresponds to 1 PFU/rxn.

FIGS. 19A-19B are schematics of PIV5 CPI based SARS-CoV-2 vaccine constructs.

### DETAILED DESCRIPTION OF THE INVENTION

### A. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure.”

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

As used herein, the term “combination” of a PIV5-based AVLP composition as described herein and at least a second pharmaceutically active ingredient means at least two, but any desired combination of compounds can be delivered simultaneously or sequentially (e.g., within a 24-hour period). It is contemplated that when used to treat various diseases, the compositions and methods of the present disclosure can be utilized with other therapeutic methods/agents suitable for the same or similar diseases. Such other therapeutic methods/agents can be co-administered (simultaneously or sequentially, in any order) to generate additive or synergistic effects. Suitable therapeutically effective dosages for each agent may be lowered due to the additive action or synergy. Also, two or more embodiments of the disclosure may be also co-administered to generate additive or synergistic effects.

The term “coronavirus” refers to a group of related RNA viruses that cause diseases in mammals and birds. In humans, these viruses cause respiratory tract infections that can range from mild to lethal. Mild illnesses include some cases of the common cold (which is caused also by certain other viruses, predominantly rhinoviruses), while more lethal varieties can cause SARS, MERS, and COVID-19. There are presently no vaccines or antiviral drugs to prevent or treat human coronavirus infections.

The term “SARS” or “severe acute respiratory syndrome” refers to a viral respiratory disease of zoonotic origin that surfaced in the early 2000s caused by severe acute respiratory syndrome coronavirus (SARS-CoV or SARS-CoV-1), the first-identified strain of the SARS coronavirus species severe acute respiratory syndrome-related coronavirus (SARS-CoV). The syndrome caused the 2002-2004 SARS outbreak. In 2019, its successor, the related virus strain Severe Acute Respiratory Syndrome Coronavirus 2 (SARS-CoV-2), was discovered.

The term “Covid-19” or “Coronavirus disease 2019” refers to a severe acute respiratory syndrome (SARS) caused by a virus known as SARS-Coronavirus 2 (SARS-CoV2).

As described herein, the term “vaccinating” designates typically the sequential administration of one or more antigens to a subject, to produce and/or enhance an immune response against the antigen(s). The sequential administration includes a priming immunization followed by one or several boosting immunizations.

Within the context of the present invention, the term “pathogen” refers to any agent that can cause a pathological condition. Examples of “pathogens” include, without limitation, cells (e.g., bacteria cells, diseased mammal cells, cancer mammal cells), fungus, parasites, viruses, prions or toxins. Preferred pathogens are infectious pathogens. In a particular embodiment, the infectious pathogen is a virus, such as the coronaviruses.

An antigen, as used therein, designates any molecule which can cause a T-cell or B-cell immune response in a subject. An antigen specific for a pathogen is, typically, an element obtained or derived from said pathogen, which contains an epitope, and which can cause an immune response against the pathogen. Depending on the pathogenic agent, the antigen may be of various nature, such as a (poly)peptide, protein, nucleic acid, lipid, cell, etc. Live weakened forms of pathogens (e.g., bacteria, viruses), or killed or inactivated forms thereof may be used as well, or purified material therefrom such as proteins, peptides, lipids, etc. The antigen may be naturally-occurring or artificially created. It may be exogenous to the treated mammal, or endogenous (e.g., tumor antigens). The antigen may be produced by techniques known per se in the art, such as for instance synthetic or recombinant technologies, or enzymatic approaches.

In a particular embodiment, the antigen is a protein, polypeptide and/or peptide. The terms “polypeptide”, “peptide” and “protein” are used interchangeably herein to refer to a polymer of amino acid residues. The terms also apply to amino acid polymers in which one or more amino acid residues may be modified or non-naturally occurring residues, such as an artificial chemical mimetic of a corresponding naturally occurring amino acid. It should be understood that the term “protein” also includes fragments or variants of different antigens, such as epitope-containing fragments, or proteins obtained from a pathogen and subsequently enzymatically, chemically, mechanically or thermally modified.

A “therapeutically effective amount” means the amount of a compound (e.g., a PIV5-based composition as described herein) that, when administered to a subject for treating a state, disorder or condition, is sufficient to effect such treatment. The “therapeutically effective amount” will vary depending on the compound or bacteria administered as well as the disease and its severity and the age, weight, physical condition and responsiveness of the mammal to be treated.

The phrase “pharmaceutically acceptable”, as used in connection with compositions of the disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “pharmaceutically acceptable composition” as used herein refers to a composition comprising at least one compound as disclosed herein formulated together with one or more pharmaceutically acceptable carriers.

The term “administration” refers to the introduction of an amount of a predetermined substance into a patient by a certain suitable method. The composition disclosed herein may be administered via any of the common routes, as long as it is able to reach a desired tissue, for example, but is not limited to, inhaling, intraperitoneal, intravenous, intramuscular, subcutaneous, intradermal, oral, topical, intranasal, intrapulmonary, or intrarectal administration. However, since peptides are digested upon oral administration, active ingredients of a composition for oral administration should be coated or formulated for protection against degradation in the stomach.

The term “dose” means a single amount of a compound or an agent that is being administered thereto; and/or “regimen: which means a plurality of pre-determined doses that can be different in amounts or similar, given at various time intervals, which can be different or similar in terms of duration. In some embodiments, a regimen also encompasses a time of a delivery period (e.g., agent administration period, or treatment period). Alternatively, a regimen is a plurality of predetermined plurality pre-determined vaporized amounts given at pre-determined time intervals.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein, the term “parainfluenza virus 5” (PIV5) includes, for example and not limitation, strains KNU-11, CC-14, D277, 1168-1, and 08-1990. Non-limiting examples of PIV5 genomes are listed in GenBank Accession Nos. NC_006430.1, AF052755.1, KC852177.1, KP893891.1, KC237065.1, KC237064.1 and KC237063.1, which are hereby incorporated by reference.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

The mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described as making up the various elements of the disclosure are intended to be illustrative and not restrictive. Many suitable materials that would perform the same or a similar function as the materials described herein are intended to be embraced within the scope of the disclosure. Such other materials not described herein can include, but are not limited to, for example, materials that are developed after the time of the development of the disclosure.

### B. SARS-COV-2 Compositions

The disclosure provides PIV5-based compositions, systems and methods for their use in multiple applications including functional genomics, drug discovery, target validation, protein production (e.g., therapeutic proteins, vaccines, monoclonal antibodies), gene therapy, and therapeutic treatments such as cancer therapy.

I. SARS-CoV-2

With the present invention, constructs of the parainfluenza virus type-5 (PIV5) virus expressing the SARS-CoV-2 envelope spike (S) and nucleocapsid (N) protein have been generated for use as vaccines against COVID. These constructs demonstrate effectiveness as vaccines, with single dose intranasal immunization inducing sterilizing immunity in ferrets and cats.

Coronavirus disease 2019 (COVID-19) is a newly emerging infectious disease currently spreading across the world. It is caused by a novel coronavirus, severe acute respiratory syndrome coronavirus 2 (SARS-CoV-2) (Zhu et al., 2020, N Engl J Med; 382:727-733). SARS-CoV-2 was first identified in Wuhan, China in December 2019, and has subsequently spread globally to cause the COVID-19 pandemic. The virus has infected more than 221 million persons world-wide, caused more than 4,574,000 deaths as of Sep. 8, 2021, and is poised to continue to spread in the absence of herd immunity. While vaccines and antibody therapies have been introduced worldwide, the emergence of multiple viral variants which are rapidly replacing the original virus identified in Wuhan has allowed for immune escape in vaccinated populations, presenting a need for improved vaccine efficacy.

SARS-CoV-2 is a single-stranded RNA-enveloped virus belonging to the B coronavirus family (Lu et al., 2020, Lancet; 395:565-74). An RNA-based metagenomic next-generation sequencing approach has been applied to characterize its entire genome, which is 29,881 nucleotides (nt) in length (GenBank Sequence Accession MN908947) encoding 9860 amino acids (Chen et al., 2020, Emerg Microbes Infect; 9:313-9). Full-genome sequenced genomes available at GenBank include isolate 2019-nCoV WHU01 (GenBank accession number MN988668) and NC_045512 for SARS-CoV-2, both isolates from Wuhan, China, and at least seven additional sequences (MN938384.1, MN975262.1, MN985325.1, MN988713.1, MN994467.1, MN994468.1, and MN997409.1) which are >99.9% identical and are hereby incorporate by reference.

1. SARS-CoV-2 Variants

Since SARS-CoV-2 was first identified in 2019, multiple genetic variants of SARS-CoV-2 have been emerging and circulating around the world. Viral mutations and variants in the United States are routinely monitored through sequence-based surveillance, laboratory studies, and epidemiological investigations. The US government SARS-CoV-2 Interagency Group (SIG) developed a Variant Classification scheme that defines three classes of SARS-CoV-2 variants: variant of interest, variant of concern and variant of high consequence.

a. Variant of Interest

A SARS-CoV-2 variant of interest is a variant with specific genetic markers that have been associated with changes to receptor binding, reduced neutralization by antibodies generated against previous infection or vaccination, reduced efficacy of treatments, potential diagnostic impact, or predicted increase in transmissibility or disease severity.

A variant of interest might require one or more appropriate public health actions, including enhanced sequence surveillance, enhanced laboratory characterization, or epidemiological investigations to assess how easily the virus spreads to others, the severity of disease, the efficacy of therapeutics and whether currently approved or authorized vaccines offer protection. The growing list variants of interest that are being monitored and characterized include, but are not limited to, Eta, Iota, Kappa, Lambda and Mu.

b. Variant of Concern

A SARS-CoV-2 variant of concern is a variant for which there is evidence of an increase in transmissibility, more severe disease (e.g., increased hospitalizations or deaths), significant reduction in neutralization by antibodies generated during previous infection or vaccination, reduced effectiveness of treatments or vaccines, or diagnostic detection failures. Possible attributes of a variant of concern include evidence of impact on diagnostics, treatments, or vaccines, widespread interference with diagnostic test targets, evidence of substantially decreased susceptibility to one or more class of therapies, evidence of significant decreased neutralization by antibodies generated during previous infection or vaccination, evidence of reduced vaccine-induced protection from severe disease, evidence of increased transmissibility and evidence of increased disease severity.

Variants of concern might require one or more appropriate public health actions, such as notification to WHO under the International Health Regulations, reporting to CDC, local or regional efforts to control spread, increased testing, or research to determine the effectiveness of vaccines and treatments against the variant. Based on the characteristics of the variant, additional considerations may include the development of new diagnostics or the modification of vaccines or treatments. The growing list of variants of concern that are being closely monitored and characterized, includes, but is not limited to, Alpha, Beta, Delta, and Gamma.

c. Variant of High Consequence

A SARS-CoV-2 variant of high consequence has clear evidence that prevention measures or medical countermeasures (MCMs) have significantly reduced effectiveness relative to previously circulating variants. Possible attributes of a variant of high consequence include a demonstrated failure of diagnostic test targets, evidence to suggest a significant reduction in vaccine effectiveness, a disproportionately high number of vaccine breakthrough cases, or very low vaccine-induced protection against severe disease, significantly reduced susceptibility to multiple Emergency Use Authorization (EUA) or approved therapeutics and more severe clinical disease and increased hospitalizations.

A variant of high consequence would require notification to WHO under the International Health Regulations, reporting to CDC, an announcement of strategies to prevent or contain transmission, and recommendations to update treatments and vaccines. Currently, there are no SARS-CoV-2 variants that rise to the level of high consequence.

II. Parainfluenza Virus 5 (PIV5)

Parainfluenza virus 5 (PIV5), a negative-stranded RNA virus, is a member of the Rubulavirus genus of the family Paramyxoviridae which includes many important human and animal pathogens such as mumps virus, human parainfluenza virus type 2 and type 4, Newcastle disease virus, Sendai virus, HPIV3, measles virus, canine distemper virus, rinderpest virus and respiratory syncytial virus. PIV5 was previously known as Simian Virus-5 (SV5). Although PIV5 is a virus that infects many animals and humans, no known symptoms or diseases in humans have been associated with PIV5. Unlike most paramyxoviruses, PIV5 infect normal cells with little cytopathic effect. As a negative stranded RNA virus, the genome of PIV5 is very stable. As PIV5 does not have a DNA phase in its life cycle and it replicates solely in cytoplasm, PIV5 is unable to integrate into the host genome. Therefore, using PIV5 as a vector avoids possible unintended consequences from genetic modifications of host cell DNAs. PIV5 can grow to high titers in cells, including Vero cells which have been approved for vaccine production by WHO and FDA. Thus, PIV5 presents many advantages as a vaccine vector.

A PIV5-based vaccine vector of the present invention may be based on any of a variety of wild type, mutant, or recombinant (rPIV5) strains. Wild type strains include, but are not limited to, the PIV5 strains W3A, WR (ATCC® Number VR-288TM), canine parainfluenza virus strain 78-238 (ATCC number VR-1573) (Evermann et al., 1980, J Am Vet Med Assoc; 177:1132-1134; and Evermann et al., 1981, Arch Virol; 68:165-172), canine parainfluenza virus strain D008 (ATCC number VR-399) (Binn et al., 1967, Proc Soc Exp Biol Med; 126:140-145), MIL, DEN, LN, MEL, cryptovirus, CPI+, CPI−, H221, 78524, T1 and SER. See, for example, Chatziandreou et al., 2004, J Gen Virol; 85(Pt 10):3007-16; Choppin, 1964, Virology: 23:224-233; and Baumgartner et al., 1987, Intervirology; 27:218-223. Additionally, PIV5 strains used in commercial kennel cough vaccines, such as, for example, BI, FD, Merck, and Merial vaccines, may be used.

III. PIV5 CPI Vectored SARS-CoV-2 Constructs

A PIV5 vaccine vector of the present invention may be constructed using any of a variety of methods, including, but not limited to, the reverse genetics system described in more detail in He et al. (Virology; 237(2):249-60, 1997). PIV5 encodes eight viral proteins. Nucleocapsid protein (NP), phosphoprotein (P) and large RNA polymerase (L) protein are important for transcription and replication of the viral RNA genome. The V protein plays important roles in viral pathogenesis as well as viral RNA synthesis. The fusion (F) protein, a glycoprotein, mediates both cell-to-cell and virus-to-cell fusion in a pH-independent manner that is essential for virus entry into cells. The structures of the F protein have been determined and critical amino acid residues for efficient fusion have been identified. The hemagglutinin-neuraminidase (HN) glycoprotein is also involved in virus entry and release from the host cells. The matrix (M) protein plays an important role in virus assembly and budding. The hydrophobic (SH) protein is a 44-residue hydrophobic integral membrane protein and is oriented in membranes with its N terminus in the cytoplasm. For reviews of the molecular biology of paramyxoviruses see, for example, Whelan et al., 2004, Curr Top Microbiol Immunol; 283:61-119; and Lamb & Parks, (2006). Paramyxoviridae: the viruses and their replication. In Fields Virology, 5th edn, pp. 1449-1496. Edited by D. M. Knipe & P. M. Howley. Philadelphia, Pa.: Lippincott Williams & Wilkins.

Previously, recombinant PIV5 viruses expressing foreign genes from numerous pathogens, including Influenza, Rabies, Respiratory Syncytial Virus, Tuberculosis, Burkholderia, and MERS-CoV have been generated and tested as vaccine candidates (Li, Z., et al., J Virol, 87(1):354 (2013); Chen, Z., et al., J Virol, 87(6): 2986 (2013); Wang, D., et al., J Virol, 91(11) (2017); Chen, Z., et al., Vaccine, 33(51):7217 (2015); Lafontaine, E. R., et al., Vaccine X., 1:100002 (2018); Li, K., et al., mBio, 11(2) (2020)). Because it actively replicates in the respiratory tract following intranasal immunization, PIV5-vectored vaccines can generate mucosal immunity that includes antigen-specific IgA antibodies and long-lived IgA plasma cells (Wang, D., et al., J Virol, 91(11) (2017). Xiao, P., et al., Front Immunol., 12:623996 (2021)). Recently a PIV5-vectored vaccine expressing the spike protein from SARS-CoV-2 Wuhan (WA1; CVXGA1) has been shown to be efficacious in mice and ferrets. A single, intranasal dose of CVXGA1 induced WA1-neutralizing antibodies and protected K18-hACE2 mice against lethal infection with SARS-CoV-2 WA1. Furthermore, a single, intranasal dose of CVXGA1 protected ferrets from infection with SARS-CoV-2 WA1 and blocked transmission to cohoused naïve ferrets (An, D., et al., Sci Adv, 7(27) (2021)). While these studies determined its efficacy against SARS-CoV-2 WA1, further studies were necessary to establish its efficacy against SARS-CoV-2 variants.

1. PIV5-Based Vaccine Vectors Encoding the SARS-CoV-2 Spike (S) Protein

With the PIV5-based vaccine vectors of the present invention, a heterologous nucleotide sequence encoding the spike (S) protein of a coronavirus, including, but not limited to, the S protein of SARS-CoV-2, is inserted in the PIV5 genome. Coronavirus entry into host cells is mediated by the transmembrane S glycoprotein (Tortorici and Veesler, 2019, Adv Virus Res; 105:93-116). As the coronavirus S glycoprotein is surface-exposed and mediates entry into host cells, it is the main target of neutralizing antibodies upon infection and the focus of therapeutic and vaccine design. The spike S protein of SARS-CoV-2 is composed of two subunits, S1 and S2. The S1 subunit contains a receptor-binding domain that recognizes and binds to the host receptor angiotensin-converting enzyme 2, while the S2 subunit mediates viral cell membrane fusion by forming a six-helical bundle via the two-heptad repeat domain. (Huang et al., 2020, Acta Pharmacol Sinica; 0:1-9 (available on the worldwide web at doi.org/10.1038/s41401-020-0485-4).

The total length of SARS-CoV-2 S is 1273 amino acids (aa) and consists of a signal peptide (amino acids 1-13) located at the N-terminus, the S1 subunit (14-685 residues), and the S2 subunit (686-1273 residues); the last two regions are responsible for receptor binding and membrane fusion, respectively. In the S1 subunit, there is an N-terminal domain (14-305 residues) and a receptor-binding domain (RBD, 319-541 residues); the fusion peptide (FP) (788-806 residues), heptapeptide repeat sequence 1 (HR1) (912-984 residues), HR2 (1163-1213 residues), TM domain (1213-1237 residues), and cytoplasm domain (1237-1273 residues) comprise the S2 subunit (Xia et al., 2020, Cell Mol Immunol; 17:765-7).

In some PIV5-based vaccine vectors of the present invention, the heterologous nucleotide sequence encoding the spike (S) protein of a coronavirus, including, but not limited to, the S protein of SARS-CoV-2, has been modified so that the cytoplasmic tail of the coronavirus S protein has been replaced with the cytoplasmic tail of the fusion (F) protein of PIV5. An example of such a PIV5 construct includes the PIV5 construct CVX-GA1, also referred to herein as CVXGA1, CVX-UGA1, pDA27, or DA27. CVXGA1, recombinant PIV5 expressing S from SARS-CoV-2 WA1, is currently under phase 1 clinical trial in the US (ClinicalTrials.gov. Phase 1 Study of Intranasal PIV5-vectored COVID-19 Vaccine Expressing SARS-CoV-2 Spike Protein in Healthy Adults (CVXGA1-001) 2021).

In some PIV5-based vaccine vectors of the present invention, the heterologous nucleotide sequence encoding the coronavirus S protein, including but not limited to, the S protein of SARS-CoV-2, has been modified so that the S protein includes an amino acid substitution at amino acid residue W886 and/or F888. In some aspects, the amino acid substitution at amino acid residue W886 includes a substitution of tryptophan (W) to arginine (R) and/or the amino acid substitution at amino acid residue W888 includes a substitution of phenylalanine (F) to arginine (R).

In some PIV5-based vaccine vectors of the present invention, the heterologous nucleotide sequence encoding the spike (S) protein of a coronavirus, including, but not limited to, the S protein of SARS-CoV-2, includes both a modification so that the cytoplasmic tail of the coronavirus S protein has been replaced with the cytoplasmic tail of the fusion (F) protein of PIV5 and includes an amino acid substitution at amino acid residue W886 and/or F888. In some aspects, the amino acid substitution at amino acid residue W886 includes a substitution of tryptophan (W) to arginine (R) and/or the amino acid substitution at amino acid residue W888 includes a substitution of phenylalanine (F) to arginine (R). An example of such a PIV5 construct includes the PIV5 construct CVX-GA2, also referred to herein as CVXGA2 or CVX-UGA2.

The heterologous nucleotide sequence encoding the coronavirus S protein, including but not limited to the S protein of SARS-CoV-2, may be inserted in any of a variety of locations in the PIV5 genome.

In some embodiments, the heterologous nucleotide sequence encoding the coronavirus S protein, including but not limited to the S protein of SARS-CoV-2, may be inserted between the small hydrophobic protein (SH) gene and the hemagglutinin-neuraminidase (HN) gene of the PIV5 genome.

The heterologous nucleotide sequence encoding the coronavirus S protein, including but not limited to the S protein of SARS-CoV-2, may be inserted between the hemagglutinin-neuraminidase (HN) and large RNA polymerase protein (L) gene of the PIV5 genome. In some embodiments, the heterologous nucleotide sequence is not inserted at a location between the hemagglutinin-neuraminidase (HN) and large RNA polymerase protein (L) gene of the PIV5 genome. In some embodiments, the heterologous nucleotide sequence is inserted at a location other than between the hemagglutinin-neuraminidase (FIN) and large RNA polymerase protein (L) gene of the PIV5 genome.

The heterologous nucleotide sequence encoding the coronavirus S protein, including but not limited to the S protein of SARS-CoV-2, may be inserted between the nucleocapsid protein (NP) gene and the V/P gene of the PIV5 genome.

The heterologous nucleotide sequence encoding the coronavirus S protein, including but not limited to the S protein of SARS-CoV-2, may be inserted between the M gene and the F gene of the PIV5 genome.

The heterologous nucleotide sequence encoding the coronavirus S protein, including but not limited to the S protein of SARS-CoV-2, may be inserted between the F gene and the SH gene of the PIV5 genome.

The heterologous nucleotide sequence encoding the coronavirus S protein, including but not limited to the S protein of SARS-CoV-2, may be inserted between the VP gene and the matrix protein (M) gene of the PIV5 genome.

The heterologous nucleotide sequence encoding the coronavirus S protein, including but not limited to the S protein of SARS-CoV-2, may be inserted between the leader sequence and the nucleocapsid protein (NP) gene of the PIV5 genome.

The heterologous nucleotide sequence encoding the coronavirus S protein, including but not limited to the S protein of SARS-CoV-2, may be inserted immediately downstream of the leader sequence of the PIV5 genome.

The heterologous nucleotide sequence encoding the coronavirus S protein, including but not limited to the S protein of SARS-CoV-2, may be inserted to replace all or part of a PIV5 gene within the PIV5 genome. For example, the heterologous nucleotide sequence may replace the F, HN, or SH gene of the PIV5 genome. A heterologous nucleotide sequence may be inserted within a PIV5 gene, resulting in the expression of a chimeric polypeptide. For example, the heterologous nucleotide sequence may be inserted within the SH gene nucleotide sequence, within the NP gene nucleotide sequence, within the V/P gene nucleotide sequence, within the M gene nucleotide sequence, within the F gene nucleotide sequence, within the HN gene nucleotide sequence, and/or within the L gene nucleotide sequence of a PIV5 genome.

The heterologous nucleotide sequence encoding the coronavirus S protein may be produced by inserting the coronavirus S protein gene from different variants into PIV5 canine parainfluenza virus (CPI) vector: CVXGA1, 3, 4, 5, 6, 13, 14 and 16.2. PIV5-based vaccine vectors encoding the SARS-CoV-2 spike (S) and nucleocapsid (N) proteins

PIV5 canine parainfluenza virus (CPI) vaccine vectors encoding SARS-COV-2 variants of concern or variants of interest are disclosed herein. The PIV5-based vaccine vectors may comprise inserting the Spike (S) protein gene from different variants into PIV5 CPI vector: CVXGA1, 3, 4, 5, 6, 13, 14 and 16.

To improve vaccine efficacy, the SARS-CoV-2 nucleocapsid (N) may be inserted between the HN and L gene junction in addition to the SARS-CoV-2 spike (S) inserted at the SH and HN junction. In studies disclosed herein, the expression of both the S and N proteins of SARS-CoV-2 have been shown to offer additional protection for the vaccine especially the cellular immune responses offered by the N protein.

Since the CPI-S+N has reduced titer/yield, the construct may be further modified by moving the SARS-CoV-2 N protein gene to the PIV5 CPI SH gene location as SH deletion has been shown not impacting virus growth. CVXGA7 was constructed to have the N and S genes from SARS-CoV-2 inserted into the CPIΔSH backbone to produce CPIΔSH-N+S.

The mutations in the V/P gene, S157F and S308A, have been shown previously to increase viral polymerase activities and improve viral titer/yield (Timani K A, Sun D, Sun M, et al. A single amino acid residue change in the P protein of parainfluenza virus 5 elevates viral gene expression. J Virol. 2008; 82(18):9123-9133. doi:10.1128/JVI.00289-08; Sun D, Luthra P, Li Z, He B. PLK1 down-regulates parainfluenza virus 5 gene expression. PLoS Pathog., 5(7):e1000525 (2009)). Mutations S157F and S308A are herein introduced into the CPI V/P gene to produce CVXGA10 (CPI-S-PLK) and CVXGA12 (CPIΔSH-S-PLK) viruses.

A PIV5 viral vaccine of the present invention may also have a mutation, alteration, or deletion in one or more of these eight proteins of the PIV5 genome. For example, a PIV5 viral expression vector may include one or more mutations, including, but not limited to any of those described herein. In some aspects, a combination of two or more (two, three, four, five, six, seven, or more) mutations may be advantageous and may demonstrated enhanced activity.

A mutation includes, but is not limited to, a mutation of the V/P gene, a mutation of the shared N-terminus of the V and P proteins, a mutation of residues 26, 32, 33, 50, 102, and/or 157 of the shared N-terminus of the V and P proteins, a mutation lacking the C-terminus of the V protein, a mutation lacking the small hydrophobic (SH) protein, a mutation of the fusion (F) protein, a mutation of the phosphoprotein (P), a mutation of the large RNA polymerase (L) protein, a mutation incorporating residues from canine parainfluenza virus, and/or a mutation that enhances syncytial formation.

A mutation may include, but is not limited to, rPIV5-V/P-CPI-, rPIV5-CPI-, rPIV5-CPI+, rPIV5V ΔC, rPIV-Rev, rPIV5-RL, rPIV5-P-S157A, rPIV5-P-S308A, rPIV5-L-A1981D and rPIV5-F-5443P, rPIV5-MDA7, rPIV5 ΔSH-CPI-, rPIV5 ΔSH-Rev, and combinations thereof.

PIV5 can infect cells productively with little cytopathic effect (CPE) in many cell types. In some cell types, PIV5 infection causes formation of syncytia, i.e., fusion of many cells together, leading to cell death. A mutation may include one or more mutations that promote syncytia formation (see, for example Paterson et al., 2000, Virology; 270:17-30).

The V protein of PIV5 plays a critical role in blocking apoptosis induced by virus. Recombinant PIV5 lacking the conserved cysteine-rich C-terminus (rPIV5V ΔC) of the V protein induces apoptosis in a variety of cells through an intrinsic apoptotic pathway, likely initiated through endoplasmic reticulum (ER)-stress (Sun et al., 2004, J Virol; 78:5068-5078). Mutant recombinant PIV5 with mutations in the N-terminus of the V/P gene products, such as rPIV5-CPI-, also induce apoptosis (Wansley and Parks, 2002, J Virol; 76:10109-10121). A mutation includes, but is not limited to, rPIV5 ΔSH, rPIV5-CPI-, rPIV5VΔC, and combinations thereof.

In some embodiments, PIV5-based vaccine vectors of the present invention are chosen from Table 4.

Also included in the present invention are virions and infectious viral particles that include a PIV5 genome including a heterologous nucleotide sequence encoding a coronavirus S protein, including but not limited to the S protein of SARS-CoV-2.

Also included in the present invention are compositions including one or more of the PIV5 viral constructs or virions, as described herein. Such a composition may include a pharmaceutically acceptable carrier. As used, a pharmaceutically acceptable carrier refers to one or more compatible solid or liquid fillers, diluents or encapsulating substances which are suitable for administration to a human or other vertebrate animal. Such a carrier may be pyrogen free. The present invention also includes methods of making and using the viral vectors and compositions described herein.

The compositions of the present disclosure may be formulated in pharmaceutical preparations in a variety of forms adapted to the chosen route of administration. One of skill will understand that the composition will vary depending on mode of administration and dosage unit.

The agents of this invention can be administered in a variety of ways, including, but not limited to, intravenous, topical, oral, intranasal, subcutaneous, intraperitoneal, intramuscular, and intratumor deliver. In some aspects, the agents of the present invention may be formulated for controlled or sustained release. One advantage of intranasal immunization is the potential to induce a mucosal immune response.

IV. PIV5 Vaccine Virus Sequences

The vaccine virus sequences from the constructs in Table 1 are listed below. The inserted N of SARS-CoV-2 sequences are lower cased and underlined and the inserted S of SARS-CoV-2 sequences are in the lower cased sequences:

1. CVL34

The nucleic acid sequence for CVL34 is:

(SEQ ID NO: 61)
ACCAGGGGGAAAACGAAGTGGTGACTCAAATCATCGAAGACCCTC
GAGATTACATAGGTCCGGAACCTATGGCCTTCGTGACCGACCTCG
AGTCAGAGTAGTTCAATAAGGACCTATCAAGTTTGGGCAATTTTT
CGTCCCCGACACAAAAATGTCATCCGTGCTTAAAGCATATGAGCG
ATTCACACTCACTCAAGAACTGCAAGATCAGAGTGAGGAAGGTAC
AATCCCACCTACAACACTAAAACCGGTAATCAGGGTATTTATACT
AACCTCTAATAACCCAGAGCTAAGATCCCGGCTTCTTCTATTCTG
CCTACGGATTGTTCTCAGTAATGGTGCAAGGGATTCCCATCGCTT
TGGAGCATTACTTACAATGTTTTCGCTACCATCAGCCACAATGCT
CAATCATGTCAAATTAGCTGACCAGTCACCAGAAGCTGATATCGA
AAGGGTAGAGATCGATGGCTTTGAGGAGGGATCATTCCGCTTAAT
CCCCAATGCTCGTTCAGGTATGAGCCGTGGAGAGATCAATGCCTA
TGCTGCACTTGCAGAAGATCTACCTGACACACTAAACCATGCAAC
ACCTTTTGTTGATTCCGAAGTCGAGGGAACTGCATGGGATGAGAT
TGAGACTTTCTTAGATATGTGTTACAGTGTCCTAATGCAGGCATG
GATAGTGACTTGCAAGTGCATGACTGCGCCAGACCAACCTGCTGC
TTCTATTGAGAAACGCCTGCAAAAATATCGTCAGCAAGGCAGGAT
CAACCCGAGATATCTCCTGCAACCGGAGGCTCGAAGAATAATCCA
GAATGTAATCCGAAAGGGAATGGTGGTCAGACATTTCCTCACCTT
TGAACTGCAGCTTGCCCGAGCACAAAGCCTTGTATCAAATAGGTA
TTATGCTATGGTAGGGGATGTTGGAAAGTATATAGAGAATTGTGG
AATGGGAGGCTTCTTTTTGACACTAAAATATGCATTAGGAACCAG
ATGGCCCACACTTGCTTTAGCTGCATTTTCAGGAGAGCTAACAAA
GCTAAAGTCCCTCATGGCATTATACCAGACCCTTGGTGAGCAGGC
CCGATATTTGGCCCTATTGGAGTCACCACATTTGATGGATTTTGC
TGCAGCAAACTACCCACTGCTATATAGCTATGCTATGGGAATAGG
CTATGTGTTAGATGTCAACATGAGGAACTACGCTTTCTCCAGATC
ATACATGAATAAGACATATTTCCAATTGGGAATGGAAACTGCAAG
AAAACAACAGGGTGCAGTTGACATGAGGATGGCAGAAGATCTCGG
TCTAACTCAAGCCGAACGCACCGAGATGGCAAATACACTTGCCAA
ATTGACCACAGCAAATCGAGGGGCAGACACCAGGGGAGGAGTCAA
CCCGTTCTCATCTATCACTGGGACAACTCAGGTGCCCGCTGCAGC
AACAGGTGACACATTCGAGAGTTACATGGCAGCGGATCGACTGAG
GCAGAGATATGCTGATGCAGGCACCCACGATGATGAGATGCCACC
ATTGGAAGAGGAGGAAGAGGACGACACATCTGCAGGTCCACGCAC
TGGACTAACTCTTGAACAAGTGGCCTTGGACATCCAGAACGCAGC
AGTTGGAGCTCCCATCCATACAGATGACCTGAATGCCGCACTGGG
TGATCTTGACATCTAGACAATTCAGATCCCAATCTTAAATCGACA
CACCTAATTGACCAGTTAGATGGAACTACAGTGGATTCCATAAGG
TTCCTGCCTACCATCGGCTTTTAAGAAAAAAATAGGCCCGGACGG
GTTAGCAACAAGCGACTGCCGATGCCAATAACACAATCCACAATC
TACAATGGATCCCACTGATCTGAGCTTCTCCCCAGATGAGATCAA
TAAGCTCATAGAGACAGGCCTGAATACTGTGGAGTATTTTACTTC
CCAACAAGTCACAGGAACATCCTCTCTTGGAAAGAATACAATACC
ACCAGGGGTCACAGGACTACTAACCAATGCTGCAGAGGCAAAGAT
CCAAGAGTCAACCAACCATCAGAAGGGTTCAGTTGGTGGGGGCGC
AAAACCAAAGAAACCGCGACCAAAAATTGCCATTGTGCCAGCAGA
TGACAAAACGGTGCCCGGAAAGCCGATCCCAAACCCTCTATTAGG
TCTGGACTCCACCCCGAGCACCCAAACTGTGCTTGATCTAAGTGG
GAAAACATTACCATCAGGATCCTATAAGGGGGTTAAACTTGCGAA
ATTTGGAAAAGAAAATCTGATGACACGGTTCATCGAGGAACCCAG
AGAGAATCCTATCGCAACCAATTCCCCCATCGATTTTAAGAGGGG
CAGGGATACCGGCGGGTTCCATAGAAGGGAGTACTCAATCGGATG
GGTGGGAGATGAAGTCAAGGTCACTGAGTGGTGCAATCCATCCTG
TTCTCCAATCACCGCTGCAGCAAGGCGATTTGAATGCACTTGTCA
CCAGTGTCCAGTCACTTGCTCTGAATGTGAACGAGATACTTAATA
CAGTGAGAAATTTGGACTCTCGGATGAATCAACTGGAGACAAAAG
TAGATCGCATTCTCTCATCTCAGTCTCTAATCCAGACCATCAAGA
ATGACATAGTTGGACTTAAAGCAGGGATGGCTACTTTAGAAGGAA
TGATTACAACTGTGAAAATCATGGACCCGGGAGTTCCCAGTAATG
TTACTGTGGAAGATGTACGCAAGAAACTAAGTAACCATGCTGTTG
TTGTGCCAGAATCATTCAATGATAGTTTCTTGACTCAATCTGAAG
ATGTAATTTCACTTGATGAGTTGGCTCGACCAACTGCAACAAGTG
TTAAGAAGATTGTCAGGAAGGTTCCTCCTCAGAAGGATCTGACTG
GATTGAAGATCACACTAGAGCAATTGGCAAAGGATTGCATCAGCA
AACCGAAGATGAGGGAAGAGTATCTCCTCAAAATCAACCAGGCTT
CCAGTGAGGCTCAGCTAATTGACCTCAAGAAAGCAATCATCCGCA
GTGCAATTTGATCAAGAAACACCCAATTACACTACACTGGTATGA
CACTGTACTAACCCTGAGGGTTTTAGAAAAAACGATTAACGATAA
ATAAGCCCGAACACTACACACCACCCGAGGCAGCCATGCCATCCA
TCAGCATCCCCGCAGACCCCACCAATCCACGTCAATCAATAAAAG
CGTTCCCAATTGTGATCAACCGTGATGGGGGTGAGAAAGGTCGCT
TGGTTAAACAACTACGCACAACCTACTTGAATGACCTAGATACTC
ATGAGCCACTGGTGACATTCGTAAATACTTATGGATTCATCTACG
AACAGGATCGGGGAAATACCATTGTCGGAGAGGATCAACATGGGA
AGAAAAGAGAGGCTGTGACTGCTGCAATGGTTACCCTTGGATGTG
GGCCTAATCTACCATCATTAGGGAATGTCCTGGGACAACTGAGTG
AATTCCAGGTCATTGTTAGGAAGACATCCAGCAAAGCGGAAGAGA
TGGTCTTTGAAATTGTTAAGTATCCGAGAATATTTCGGGGTCATA
CATTAATCCAGAAAGGACTAGTCTGTGTCTCCGCAGAAAAATTTG
TTAAGTCACCAGGGAAAGTACAATCTGGAATGGACTATCTCTTCA
TTCCGACATTTCTGTCAGTGACTTACTGTCCAGCTGCAATCAAAT
TTCAGGTACCTGGCCCCATGTTGAAAATGAGATCAAGATACACTC
AGAGCTTACAACTTGAACTAATGATAAGAATCCTGTGTAAGCCCG
ATTCGCCACTTATGAAGGTCCATATCCCTGACAAGGAAGGAAGAG
GATGTCTTGTATCAGTATGGTTGCATGTATGCAATATCTTCAAAT
CAGGAAACAAGAATGGCAGTGAGTGGCAGGAATACTGGATGAGAA
AGTGTGCTAACATGCAACTTGAAGTGTCGATTGCAGATATGTGGG
GACCAACTATCATAATTCATGCCAGAGGTCACATTCCCAAAAGTG
CTAAGTTGTTTTTTGGAAAGGGTGGATGGAGCTGCCATCCACTTC
ACGAAGTTGTTCCAAGTGTCACTAAAACACTATGGTCCGTGGGCT
GTGAGATTACAAAGGCGAAGGCAATAATACAAGAGAGTAGCATCT
CTCTTCTCGTGGAGACTACTGACATCATAAGTCCAAAAGTCAAAA
TTTCATCTAAGCATCGCCGCTTTGGGAAATCAAATTGGGGTCTGT
TCAAGAAAACCAAATCACTGCCCAACCTGACGGAGCTGGAATGAC
TGACCCCCAATCGAGACTACACCACCTCAAACTATAGGTGGGTGG
TACCTCAGTGATTAATCTCGTAAGCACTGATCGTAGGCTACAACA
CACTAATATTATCCAGATTAGAGAGCTTAATTAGCTCTGTATTAA
TAATAACACTACTATTCCAATAACTGGAATCACCAGCTTGATTTA
TCTCCAAAATGATTCAAAGAAAACAAATCATATTAAGACTATCCT
AAGCACGAACCCATATCGTCCTTCAAATCATGGGTACTATAATTC
AATTTCTGGTGGTCTCCTGTCTATTGGCAGGAGCAGGCAGTCTTG
ATCCAGCAGCCCTCATGCAAATCGGTGTCATTCCAACAAATGTCC
GGCAACTTATGTATTATACTGAGGCCTCATCAGCATTCATTGTTG
TGAAGTTAATGCCTACAATTGACTCGCCGATTAGTGGATGTAATA
TAACATCAATTTCAAGCTATAATGCAACAGTGACAAAACTCCTAC
AGCCGATCGGTGAGAATTTGGAGACGATTAGGAACCAGTTGATTC
CAACTCGGAGGAGACGCCGGTTTGCAGGGGTGGTGATTGGATTAG
CTGCATTAGGAGTAGCTACTGCCGCACAGGTCACTGCCGCAGTAG
CACTAGTAAAGGCAAATGAAAATGCTGCGGCTATACTCAATCTCA
AAAATGCAATCCAAAAAACAAATGCAGCAGTTGCAGATGTGGTCC
AGGCCACACAATCACTAGGAACAGCAGTTCAAGCAGTTCAAGATC
ACATAAACAGTGTGGTAAGTCCAGCAATTACAGCAGCCAATTGTA
AAGCCCAAGATGCTATCATTGGCTCAATCCTCAATCTCTATTTGA
CCGAGTTGACAACTATCTTCCACAATCAAATTACAAACCCTGCAT
TGAGTCCTATTACAATTCAAGCTTTAAGGATCCTACTAGGGAGTA
CCTTGCCGACTGTGGTCGAAAAATCTTTCAATACCCAGATAAGTG
CGGCTGAGCTTCTCTCATCAGGGTTATTGACAGGCCAGATTGTGG
GATTAGATTTGACCTATATGCAGATGGTCATAAAAATTGAGCTGC
CAACTTTAACTGTACAACCTGCAACCCAGATCATAGATCTGGCCA
CCATTTCTGCATTCATTAACAATCAAGAAGTCATGGCCCAATTAC
CAACACGTGTTATTGTGACTGGCAGCTTGATCCAAGCCTATCCCG
CATCGCAATGCACTATTACACCCAACACTGTGTACTGTAGGTATA
ATGATGCCCAAGTACTCTCAGATGATACGATGGCTTGCCTCCAAG
GTAACTTGACAAGATGCACCTTCTCTCCAGTGGTTGGGAGCTTTC
TCACTCGATTCGTGCTGTTCGATGGAATAGTTTATGCAAATTGCA
GGTCGATGTTGTGCAAGTGCATGCAGCCTGCTGCTGTGATCCTAC
AGCCGAGTTCATCCCCTGTAACTGTCATTGACATGTACAAATGTG
TGAGTCTGCAGCTTGACAATCTCAGATTCACCATCACTCAATTGG
CCAATGTAACCTACAATAGCACCATCAAGCTTGAAACATCCCAGA
TCTTGCCTATTGATCCGTTGGATATATCCCAGAATCTAGCTGCGG
TGAATAAGAGTCTAAGTGATGCACTACAACACTTAGCACAAAGTG
ACACATACCTTTCTGCAATCACATCAGCTACGACTACAAGTGTAT
TATCCATAATAGCAATCTGTCTTGGATCGTTAGGTTTAATATTAA
TAATCTTGCTCAGTGTAGTTGTGTGGAAGTTATTGACCATTGTCG
CTGCTAATCGAAATAGAATGGAGAATTTTGTTTATCATAATTCAG
CATTCCACCACTCACGATCTGATCTCAGTGAGAAAAATCAACCTG
CAACTCTTGGAACAAGATAAGACAGTCATCCATTAGTAATTTTTA
AGAAAAAAACGATAGGACCGAAACTAGTATTGAAAGAACCGTCTC
GGTCAATCTAGGTAATCGAGCTGCTACCGTCTCGGAAAGCTCAAA
TCATGCTGCCTGATCCGGAAGATCCGGAAAGCAAAAAAGCTACAA
GGAGAACAGGAAACCTAATTATCTGCTTCCTATTCATCTTCTTTC
CGTTTGTAAACTTCATTGTTCCAACTCTAAGACACTTGCTGTCCT
AACACCTGCTATAGGCTATCCACTGCATCATCTCTTTTTAAGAAA
AAAATAGGCCCGGACGGGTTAGCAACAAGCGACTGCCGGTGCCAA
CAACACAATCCACAATCTACAGTCGACGCGGCCGCCatgttcgtc
ttcctggtcctgctgcctctggtctcctcacagtgcgtcaatctg
acaactcggactcagctgccacctgcttatactaatagcttcacc
agaggcgtgtactatcctgacaaggtgtttagaagctccgtgctg
cactctacacaggatctgtttctgccattctttagcaacgtgacc
tggttccacgccatccacgtgagcggcaccaatggcacaaagcgg
ttcgacaatcccgtgctgccttttaacgatggcgtgtacttcgcc
tctaccgagaagagcaacatcatcagaggctggatctttggcacc
acactggactccaagacacagtctctgctgatcgtgaacaatgcc
accaacgtggtcatcaaggtgtgcgagttccagttttgtaatgat
cccttcctgggcgtgtactatcacaagaacaataagagctggatg
gagtccgagtttagagtgtattctagcgccaacaactgcacattt
gagtacgtgagccagcctttcctgatggacctggagggcaagcag
ggcaatttcaagaacctgagggagttcgtgtttaagaatatcgac
ggctacttcaaaatctactctaagcacacccccatcaacctggtg
cgcgacctgcctcagggcttcagcgccctggagcccctggtggat
ctgcctatcggcatcaacatcacccggtttcagacactgctggcc
ctgcacagaagctacctgacacccggcgactcctctagcggatgg
accgccggcgctgccgcctactatgtgggctacctccagccccgg
accttcctgctgaagtacaacgagaatggcaccatcacagacgca
gtggattgcgccctggaccccctgagcgagacaaagtgtacactg
aagtcctttaccgtggagaagggcatctatcagacatccaatttc
agggtgcagccaaccgagtctatcgtgcgctttcctaatatcaca
aacctgtgcccatttggcgaggtgttcaacgcaacccgcttcgcc
agcgtgtacgcctggaataggaagcggatcagcaactgcgtggcc
gactatagcgtgctgtacaactccgcctctttcagcacctttaag
tgctatggcgtgtcccccacaaagctgaatgacctgtgctttacc
aacgtctacgccgattctttcgtgatcaggggcgacgaggtgcgc
cagatcgcccccggccagacaggcaagatcgcagactacaattat
aagctgccagacgatttcaccggctgcgtgatcgcctggaacagc
aacaatctggattccaaagtgggcggcaactacaattatctgtac
cggctgtttagaaagagcaatctgaagcccttcgagagggacatc
tctacagaaatctaccaggccggcagcaccccttgcaatggcgtg
gagggctttaactgttatttcccactccagtcctacggcttccag
cccacaaacggcgtgggctatcagccttaccgcgtggtggtgctg
agctttgagctgctgcacgccccagcaacagtgtgcggccccaag
aagtccaccaatctggtgaagaacaagtgcgtgaacttcaacttc
aacggcctgaccggcacaggcgtgctgaccgagtccaacaagaag
ttcctgccatttcagcagttcggcagggacatcgcagataccaca
gacgccgtgcgcgacccacagaccctggagatcctggacatcaca
ccctgctctttcggcggcgtgagcgtgatcacacccggcaccaat
acaagcaaccaggtggccgtgctgtatcaggacgtgaattgtacc
gaggtgcccgtggctatccacgccgatcagctgaccccaacatgg
cgggtgtacagcaccggctccaacgtcttccagacaagagccgga
tgcctgatcggagcagagcacgtgaacaattcctatgagtgcgac
atcccaatcggcgccggcatctgtgcctcttaccagacccagaca
aactctcccagaagagcccggagcgtggcctcccagtctatcatc
gcctataccatgtccctgggcgccgagaacagcgtggcctactct
aacaatagcatcgccatcccaaccaacttcacaatctctgtgacc
acagagatcctgcccgtgtccatgaccaagacatctgtggactgc
acaatgtatatctgtggcgattctaccgagtgcagcaacctgctg
ctccagtacggcagcttttgtacccagctgaatagagccctgaca
ggcatcgccgtggagcaggataagaacacacaggaggtgttcgcc
caggtgaagcaaatctacaagaccccccctatcaaggactttggc
ggcttcaatttttcccagatcctgcctgatccatccaagccttct
aagcggagctttatcgaggacctgctgttcaacaaggtgaccctg
gccgatgccggcttcatcaagcagtatggcgattgcctgggcgac
atcgcagccagggacctgatctgcgcccagaagtttaatggcctg
accgtgctgccacccctgctgacagatgagatgatcgcacagtac
acaagcgccctgctggccggcaccatcacatccggaTggaccTTc
ggcgcaggagccgccctccagatcccctttgccatgcagatggcc
tataggttcaacggcatcggcgtgacccagaatgtgctgtacgag
aaccagaagctgatcgccaatcagtttaactccgccatcggcaag
atccaggacagcctgtcctctacagccagcgccctgggcaagctc
caggatgtggtgaatcagaacgcccaggccctgaataccctggtg
aagcagctgagcagcaacttcggcgccatctctagcgtgctgaat
gacatcctgagccggctggacaaggtggaggcagaggtgcagatc
gaccggctgatcaccggccggctccagagcctccagacctatgtg
acacagcagctgatcagggccgccgagatcagggccagcgccaat
ctggcagcaaccaagatgtccgagtgcgtgctgggccagtctaag
agagtggacttttgtggcaagggctatcacctgatgtccttccct
cagtctgccccacacggcgtggtgtttctgcacgtgacctacgtg
cccgcccaggagaagaacttcaccacagcccctgccatctgccac
gatggcaaggcccactttccaagggagggcgtgttcgtgtccaac
ggcacccactggtttgtgacacagcgcaatttctacgagccccag
atcatcaccacagacaacaccttcgtgagcggcaactgtgacgtg
gtcatcggcatcgtgaacaataccgtgtatgatccactccagccc
gagctggacagctttaaggaggagctggataagtatttcaagaat
cacacctcccctgacgtggatctgggcgacatcagcggcatcaat
gcctccgtggtgaacatccagaaggagatcgaccgcctgaacgag
gtggctaagaatctgaacgagagcctgatcgacctccaggagctg
ggcaagtatgagcagtacatcaagtggccctggtacatctggctg
ggcttcatcgccggcctgatcgccatcgtgatggtgaccatcatg
ctgtgctgtatgacatcctgctgttcttgcctgaagggctgctgt
agctgtggctcctgctgtAAGTTATTGACCATTGTCGTTGCTAAT
CGAAATAGAATGGAGAATTTTGTTTATCATAAATGAACGCGTGCT
AGCCTCCTGCCATACTTCCTACTCACATCATATCTATTTTAAAGA
AAAAATAGGCCCGAACACTAATCGTGCCGGCAGTGCCACTGCACA
CACAACACTACACATACAATACACTACAATGGTTGCAGAAGATGC
CCCTGTTAGGGGCACTTGCCGAGTATTATTTCGAACAACAACTTT
AATTTTTCTATGCACACTATTAGCATTAAGCATCTCTATCCTTTA
TGAGAGTTTAATAACCCAAAAGCAAATCATGAGCCAAGCAGGCTC
AACTGGATCTAATTCTAGATTAGGAAGTATCACTGATCTTCTTAA
TAATATTCTCTCTGTCGCAAATCAGATTATATATAACTCTGCAGT
CGCTCTACCTCTACAATTGGACACTCTTGAATCAACACTCCTTAC
AGCCATTAAGTCTCTTCAAACAAGTGACAAGCTAGAACAGAACTG
CTCGTGGGGTGCTGCACTGATTAATGATAATAGATACATTAATGG
CATCAATCAGTTCTATTTCTCAATTGCTGAGGGTCGCAAGCTGAC
ACTTGGCCCACTTCTTAATATACCTAGTTTCATTCCAACTGCCAC
GACACCAGAGGGCTGCACCAGGATCCCATCATTCTCGCTCACCAA
GACACACTGGTGTTATACACACAATGTTATCCTGAATGGATGCCA
GGATCATGTATCCTCAAATCAATTTGTTTCCATGGGAATCATTGA
ACCCACTTCTGCCGGGTTTCCATCCTTTCGAACCCTAAAGACTCT
ATATCTCAGCGATGGGGTCAATCGTAAGAGCTGCTCTATCAGTAC
AGTTCCGGGGGGTTGTATGATGTACTGTTTCGTCTCTACTCAACC
AGAGAGGGATGACTACTTTTCTACCGCTCCTCCAGAACAACGAAT
TATTATAATGTACTATAATGATACAATCGTGGAGCGCATAATTAA
TCCACCCGGGGTACTAGATGTATGGGCAACATTGAACCCAGGAAC
AGGAAGCGGGGTATATTATTTAGGTTGGGTACTCTTTCCAATATA
TGGCGGCGTGATTAAAGATACGAGTTTATGGAATAATCAAGCAAA
TAAATACTTTATCCCCCAGATGGTTGCTGCTCTCTGCTCACAAAA
CCAGGCAACTCAAGTCCAAAATGCTAAGTCATCATACTATAGCAG
CTGGTTTGGCAATCGAATGATTCAGTCTGGGATCCTGGCATGTCC
TCTTCAACAGGATCTAACCAATGAGTGTTTAGTTCTGCCCTTTTC
TAATGATCAGGTGCTTATGGGTGCTGAAGGGAGATTATACATGTA
TGGTGACTCGGTGTATTACTACCAAAGAAGCAATAGTTGGTGGCC
TATGACCATGCTGTATAAGGTAACCATAACATTCACTAATGGTCA
GCCATCTGCTATATCAGCTCAGAATGTGCCCACACAGCAGGTCCC
TAGACCTGGGACAGGAAGCTGCTCTGCAACAAATAGATGTCCCGG
TTTTTGCTTGAAAGGAGTGTATGCTGATGCCTGGTTACTGACCAA
CCCTTCGTCTACCAGTACATTTGGATCAGAAGCAACCTTCACTGG
TTCTTATCTCAACGCAGCAACTCAGCGTATCAATCCGACGATGTA
TATCGCGAACAACACACAGATCATAAGCTCACAGCAATTTGGATC
AAGCGGTCAAGAAGCAGCATATGGCCACACAACTTGTTTTAGGGA
CACAGGCTCTGTTATGGTATACTGTATCTATATTATTGAATTGTC
CTCATCTCTCTTAGGACAATTTCAGATTGTCCCATTTATCCGTCA
GGTGACACTATCCTAAAGGCAGAAGCCTTCAGGTCTGACCCAGCC
AATCAAAGCATTATACCAGACCATGGCCTACCATCGGCTTTAAAG
AAAAAAATAGGCCCGGACGGGTTAGCAACAAGCGGCGGCCGCGAA
TTCGCCACCatgtcagataacggaccacagaaccagaggaacgca
cccaggattactttcggaggaccaagcgatagcaccgggagcaac
cagaatggagagcggagcggagcaagatccaagcagagacggccc
cagggcctgccaaacaataccgcatcctggttcaccgccctgaca
cagcacggcaaggaggacctgaagtttccaaggggacagggagtg
cctatcaacaccaatagctcccctgacgatcagatcggctactat
aggagggcaacaaggagaatcaggggaggcgacggcaagatgaag
gatctgagcccacgctggtacttctactatctgggaaccggacct
gaggcaggcctgccatatggcgccaacaaggacggaatcatctgg
gtggcaaccgagggcgccctgaacacaccaaaggatcacatcggc
acaagaaatcccgccaacaatgcagcaatcgtgctgcagctgcca
cagggaaccacactgcccaagggcttttacgcagagggctctcgg
ggaggcagccaggcatctagcagatcctctagccggagcagaaac
tcctctaggaattccaccccaggaagctccaggggcacatcccct
gcccgcatggcaggaaacggaggcgacgccgccctggccctgctg
ctgctggatcgcctgaatcagctggagtccaagatgtctggcaag
ggacagcagcagcagggacagaccgtgacaaagaagtccgccgcc
gaggcctctaagaagccaaggcagaagcgcaccgccacaaaggcc
tacaacgtgacccaggccttcggcaggcgcggaccagagcagaca
cagggcaattttggcgaccaggagctgatcaggcagggaaccgat
tataagcactggcctcagatcgcccagttcgccccatctgccagc
gccttctttggcatgtctagaatcggcatggaggtgacccccagc
ggcacatggctgacctacacaggcgccatcaagctggacgataag
gaccctaacttcaaggatcaggtcatcctgctgaacaagcacatc
gacgcctataagacctttccccctacagagcccaagaaggacaag
aagaagaaggccgatgagacacaggccctgcctcagaggcagaag
aagcagcagaccgtgacactgctgccagccgccgatctggacgat
ttctccaaacagctgcagcagagcatgtccagtgccgactccacc
caggcttgatgaTGAGGTACCTGCGGCCGCCTAAGGTCGACTCAT
GGAATGCATACCAAACATTATTGACACTAATGACACACAAAATTG
GTTTTAAGAAAAACCAAGAGAACAATAGGCCAGAATGATGGCTGG
GTCTCGGGAGATATTACTCCCTGAAGTCCATCTCAATTCACCAAT
TGTAAAGCATAAGCTATACTATTACATTCTACTTGGAAACCTCCC
AAATGAGATCGACATTGACGATTTAGGTCCATTACATAATCAAAA
TTGGAATCAAATAGCACATGAAGAGTCTAACTTAGCCCAACGCTT
GGTAAATGTAAGAAATTTTCTAATTACCCACATCCCTGATCTTAG
AAAGGGCCATTGGCAAGAGTATGTCAATGTAATACTGTGGCCGCG
AATTCTTCCCTTGATCCCGGATTTTAAAATCAATGACCAATTGCC
TCTACTCAAAAATTGGGACAAGTTAGTTAAAGAATCATGTTCAGT
AATCAATGCGGGTACTTCCCAGTGCATTCAGAATCTCAGCTATGG
ACTGACAGGTCGTGGGAACCTCTTTACACGATCACGTGAACTCTC
TGGTGACCGCAGGGATATTGATCTTAAGACGGTTGTGGCAGCATG
GCATGACTCAGACTGGAAAAGAATAAGTGATTTTTGGATTATGAT
CAAATTCCAGATGAGACAATTAATTGTTAGGCAAACAGATCATAA
TGATCCTGATTTAATCACGTATATCGAAAATAGAGAAGGCATAAT
CATCATAACCCCTGAACTGGTAGCATTATTTAACACTGAGAATCA
TACACTAACATACATGACCTTTGAAATTGTACTGATGGTTTCAGA
TATGTACGAAGGTCGTCACAACATTTTATCACTATGCACAGTTAG
CACTTACCTGAATCCTCTGAAGAAAAGAATAACATATTTATTGAG
CCTTGTAGATAACTTAGCTTTTCAGATAGGTGATGCTGTATATAA
CATAATTGCTTTGCTAGAATCCTTTGTATATGCACAGTTGCAAAT
GTCAGATCCCATCCCAGAACTCAGAGGACAATTCCATGCATTCGT
ATGTTCTGAGATTCTTGATGCACTAAGGGGAACTAATAGTTTCAC
CCAGGATGAATCAAGAACTGTGACAACCAATTTGATATCCCCATT
CCAAGATCTGACCCCAGATCTTACGGCTGAATTGCTCTGTATAAT
GAGGCTTTGGGGACACCCCATGCTCACCGCCAGTCAAGCTGCGGG
AAAGGTACGCGAGTCCATGTGTGCTGGAAAAGTATTAGACTTTCC
CACCATTATGAAAACACTAGCCTTTTTCCATACTATTCTGATCAA
TGGATACAGGAGGAAGCATCATGGAGTATGGCCACCCTTAAACTT
ACCGGGTAATGCTTCAAAGGGTCTCACGGAACTTATGAATGACAA
TACTGAGATAAGCTATGAATTCACACTTAAGCATTGGAAGGAAAT
CTCTCTTATAAAATTCAAGAAATGTTTTGATGCAGACGCAGGTGA
GGAACTCAGTATATTTATGAAAGATAAAGCAATTAGTGCCCCAAA
ACAAGATTGGATGAGTGTGTTTAGAAGAAGCCTAATCAAACAGCG
CCATCAGCATCATCAGGTCCCCCTACCAAATCCATTCAATCGACG
GCTATTGCTAAACTTTCTCGGAGATGACAAATTCGACCCGAATGT
GGAGCTACAGTATGTAACATCAGGTGAGTATCTACATGATGACAC
GTTTTGTGCATCATATTCACTAAAAGAGAAGGAAATTAAACCTGA
TGGTCGAATTTTTGCAAAGTTGACTAAGAGAATGAGATCATGTCA
AGTTATAGCAGAATCTCTTTTAGCGAACCATGCTGGGAAGTTAAT
GAAAGAGAATGGTGTTGTGATGAATCAGCTATCATTAACAAAATC
ACTATTAACAATGAGTCAGATTGGAATAATATCCGAGAAAGCTAG
AAAATCGACTCGAGATAACATAAATCAACCTGGTTTCCAGAATAT
CCAGAGAAATAAATCACATCACTCCAAGCAAGTCAATCAGCGAGA
TCCAAGTGATGACTTTGAATTGGCAGCATCTTTTTTAACTACTGA
TCTCAAAAAATATTGTTTACAATGGAGGTACCAGACAATTATCCC
ATTTGCTCAATCATTAAACAGAATGTATGGTTATCCTCATCTCTT
TGAGTGGATTCACTTACGGCTAATGCGTAGTACACTTTACGTGGG
GGATCCCTTCAACCCACCAGCAGATACCAGTCAATTTGATCTAGA
TAAAGTAATTAATGGAGATATCTTCATTGTATCACCCAGAGGTGG
AATTGAAGGGCTATGTCAAAAGGCTTGGACAATGATATCTATCTC
TGTGATAATTCTATCTGCCACAGAGTCTGGCACACGAGTAATGAG
TATGGTGCAGGGAGATAATCAAGCAATTGCTGTCACCACACGAGT
ACCAAGGAGCCTGCCGACTCTTGAGAAAAAGACTATTGCTTTTAG
ATCTTGTAATCTATTCTTTGAGAGGTTAAAATGTAATAATTTTGG
ATTAGGTCACCATTTGAAAGAACAAGAGACTATCATTAGTTCTCA
CTTCTTTGTTTATAGCAAGAGAATATTCTATCAGGGGAGGATTCT
AACGCAAGCCTTAAAAAATGCTAGTAAGCTCTGCTTGACAGCTGA
TGTCCTAGGAGAATGTACCCAATCATCATGTTCTAATCTTGCAAC
TACTGTCATGAGGTTAACTGAGAATGGTGTTGAAAAAGATATCTG
TTTCTACTTGAATATCTATATGACCATCAAACAGCTCTCCTATGA
TATCATCTTCCCTCAAGTGTCAATTCCTGGAGATCAGATCACATT
AGAATACATAAATAATCCACACCTGGTATCACGATTGGCTCTTCT
GCCATCCCAGCTAGGAGGTCTAAACTACCTGTCATGCAGTAGGCT
GTTCAATCGAAACATAGGCGACCCGGTGGTTTCCGCAGTTGCAGA
TCTTAAGAGATTAATTAAATCAGGATGTATGGATTACTGGATCCT
TTATAACTTATTAGGGAGAAAACCGGGAAACGGCTCATGGGCTAC
TTTAGCAGCTGACCCGTACTCAATCAATATAGAGTATCAATACCC
CCCAACTACAGCTCTTAAGAGGCACACCCAACAAGCTCTGATGGA
ACTCAGTACGAATCCAATGTTACGTGGCATATTCTCTGACAATGC
ACAGGCAGAAGAAAATAATCTTGCTAGATTTCTCCTGGATAGGGA
GGTGATCTTTCCGCGTGTAGCTCACATCATCATTGAGCAAACCAG
TGTCGGGAGGAGAAAACAGATTCAAGGATATTTGGATTCAACTAG
ATCGATAATGAGTAAATCACTAGAAATTAAGCCCTTGTCCAATAG
GAAGCTTAATGAAATACTGGATTACAACATCAATTACCTAGCTTA
CAATTTGGCATTACTCAAGAATGCTATTGAACCTCCGACTTATTT
GAAAGCAATGACTCTTGAAACATGTAGCATCGACATTGCAAGGAG
CCTCCGGAAGCTCTCCTGGGCCCCACTCTTGGGTGGGAGAAATCT
TGAAGGATTAGAGACGCCAGATCCCATTGAAATTACTGCAGGAGC
ATTAATTGTTGGATCGGGCTACTGTGAACAGTGTGCTGCAGGAGA
CAATCGATTCACATGGTTTTTCTTGCCATCTGGTATCGAGATAGG
AGGGGATCCCCGTGATAATCCTCCTATCCGTGTACCGTACATTGG
CTCCAGGACTGATGAGAGGAGGGTAGCCTCAATGGCATACATCAG
GGGTGCCTCGAGTAGCCTAAAAGCAGTTCTTAGACTGGCGGGAGT
GTACATCTGGGCATTCGGAGATACTCTGGAGAATTGGATAGATGC
ACTGGATTTGTCTCACACTAGAGTTAACATCACACTTGAACAGCT
GCAATCCCTCACCCCACTTCCAACCTCTGCCAATCTAACCCATCG
GTTGGATGATGGCACAACTACCCTAAAGTTTACTCCTGCGAGCTC
TTACACCTTTTCAAGTTTCACTCATATATCAAATGATGAGCAATA
CCTGACAATTAATGACAAAACTGCAGATTCAAATATAATCTACCA
ACAGTTAATGATCACTGGACTCGGAATTTTAGAAACATGGAATAA
TCCCCCAATCAATAGAACATTCGAAGAATCTACCCTACATTTGCA
CACTGGTGCATCATGTTGTGTCCGACCTGTGGACTCCTGCATCAT
CTCAGAAGCATTAACAGTCAAGCCACATATTACAGTACCGTACAG
CAATAAATTTGTATTTGATGAAGACCCGCTATCTGAATATGAGAC
TGCAAAACTGGAATCGTTATCATTTCAAGCCCAATTAGGCAACAT
TGATGCTGTAGATATGACAGGTAAATTAACATTATTGTCCCAATT
CACTGCAAGGCAGATTATTAATGCAATCACTGGACTCGATGAGTC
TGTCTCTCTTACTAATGATGCCATTGTTGCATCAGACTATGTCTC
CAATTGGATTAGTGAATGCATGTATACCAAATTAGATGAATTATT
TATGTATTGTGGGTGGGAACTACTATTGGAACTATCCTATCAAAT
GTATTATCTGAGGGTAGTTGGGTGGAGTAATATAGTGGATTATTC
TTACATGATCTTGAGAAGAATCCCGGGTGCAGCATTAAACAATCT
GGCATCTACATTAAGTCATCCAAAACTTTTCCGACGAGCTATCAA
CCTAGATATAGTTGCCCCCTTAAATGCTCCTCATTTTGCATCTCT
GGACTACATCAAGATGAGTGTGGATGCAATACTCTGGGGCTGTAA
AAGAGTCATCAATGTGCTCTCCAATGGAGGGGACTTAGAATTAGT
TGTGACATCTGAAGATAGCCTTATTCTCAGTGACCGATCCATGAA
TCTCATTGCAAGGAAATTAACTTTATTATCACTGATTCACCATAA
TGGTTTGGAACTACCAAAGATTAAGGGGTTCTCTCCTGATGAGAA
GTGTTTCGCTTTGACAGAATTTTTGAGGAAAGTGGTGAACTCAGG
GTTGAGTTCAATAGAGAACCTATCAAATTTTATGTACAATGTGGA
GAACCCACGGCTTGCAGCATTCGCCAGCAACAATTACTACCTGAC
CAGAAAATTATTGAATTCAATACGAGATACTGAGTCGGGTCAAGT
AGCAGTCACCTCATATTATGAATCATTAGAATATATTGATAGTCT
TAAGCTAACCCCACATGTGCCTGGTACCTCATGCATTGAGGATGA
TAGTCTATGTACAAATGATTACATAATCTGGATCATAGAGTCTAA
TGCAAACTTGGAGAAGTATCCAATTCCAAATAGCCCTGAGGATGA
TTCCAATTTCCATAACTTTAAGTTGAATGCTCCATCGCACCATAC
CTTACGCCCATTAGGGTTGTCATCAACTGCTTGGTATAAGGGTAT
AAGCTGTTGCAGGTACCTTGAGCGATTAAAGCTACCACAAGGTGA
TCATTTATATATTGCAGAAGGTAGTGGTGCCAGTATGACAATCAT
AGAATACCTATTCCCAGGAAGAAAGATATATTACAATTCTTTATT
TAGTAGTGGTGACAATCCCCCACAAAGAAATTATGCACCAATGCC
TACTCAGTTCATTGAGAGTGTCCCATACAAGCTCTGGCAAGCACA
CACAGATCAATATCCCGAGATTTTTGAGGACTTCATCCCTCTATG
GAACGGAAATGCCGCCATGACTGACATAGGAATGACAGCTTGTGT
AGAATTCATCATCAATCGAGTCGGCCCAAGGACTTGCAGTTTAGT
ACATGTAGATTTGGAATCAAGTGCAAGCTTAAATCAACAATGCCT
GTCAAAGCCGATAATTAATGCTATCATCACTGCTACAACTGTTTT
GTGCCCTCATGGGGTGCTTATTCTGAAATATAGTTGGTTGCCATT
TACTAGATTTAGTACTTTGATCACTTTCTTATGGTGCTACTTTGA
GAGAATCACTGTTCTTAGGAGCACATATTCTGGTCCAGCTAATCA
TGAGGTTTATTTAATTTGTATCCTTGCCAACAACTTTGCATTCCA
GACTGTCTCGCAGGCAACAGGAATGGCGATGACTTTAACCGATCA
AGGGTTTACTTTGATATCACCTGAAAGAATAAATCAGTATTGGGA
TGGTCACTTGAAGCAAGAACGTATCGTAGCAGAAGCAATTGATAA
GGTGGTTCTAGGAGAAGATGCTCTATTCAATTCGAGTGATAATGA
ATTAATTCTCAAATGTGGAGGGACACCAAATGCACGGAATCTTAT
CGATATCGAGCCAGTCGCAACTTTCATAGAATTTGAACAACTGAT
CTGCACAATGTTGACAACCCACTTGAAGGAAATAATTGATATAAC
AAGGTCTGGAACCCAGGATTATGAAAGTTTATTACTCACTCCTTA
CAATTTAGGTCTTCTTGGTAAAATCAGTACGATAGTGAGATTATT
AACAGAAAGGATTCTAAATCATACTATCAGGAATTGGTTGATCCT
CCCACCTTCGCTCCGGATGATCGTGAAGCAGGACTTGGAATTCGG
CATATTCAGGATTACTTCCATCCTCAATTCTGATCGGTTCCTGAA
GCTTTCTCCAAATAGGAAATACTTGATTACACAATTAACTGCAGG
CTACATTAGGAAATTGATTGAGGGGGATTGTAATATCGATCTAAC
CAGACCTATCCAAAAACAAATCTGGAAAGCATTAGGTTGTGTAGT
CTATTGTCACGATCCAGTAGATCAAAGGGAATCAACAGAGTTTAT
TGATATAAATATTAATGAAGAAATAGACCGCGGGATCGATGGCGA
GGAAATCTAAATATATCAAGAATCAGAATTAGTTTAAGAAAAAAG
AAGAGGATTAATCTTGGTTTTCCCCTTGGT.

2. CVL44

The nucleic acid sequence for CVL44 is:

(SEQ ID NO: 62)
ACCAGGGGGAAAACGAAGTGGTGACTCAAATCATC
GAAGACCCTCGAGATTACATAGGTCCGGAACCTAT
GGCCTTCGTGACCGACCTCGAGTCAGAGTAGTTCA
ATAAGGACCTATCAAGTTTGGGCAATTTTTCGTCC
CCGACACAAAAATGTCATCCGTGCTTAAAGCATAT
GAGCGATTCACACTCACTCAAGAACTGCAAGATCA
GAGTGAGGAAGGTACAATCCCACCTACAACACTAA
AACCGGTAATCAGGGTATTTATACTAACCTCTAAT
AACCCAGAGCTAAGATCCCGGCTTCTTCTATTCTG
CCTACGGATTGTTCTCAGTAATGGTGCAAGGGATT
CCCATCGCTTTGGAGCATTACTTACAATGTTTTCG
CTACCATCAGCCACAATGCTCAATCATGTCAAATT
AGCTGACCAGTCACCAGAAGCTGATATCGAAAGGG
TAGAGATCGATGGCTTTGAGGAGGGATCATTCCGC
TTAATCCCCAATGCTCGTTCAGGTATGAGCCGTGG
AGAGATCAATGCCTATGCTGCACTTGCAGAAGATC
TACCTGACACACTAAACCATGCAACACCTTTTGTT
GATTCCGAAGTCGAGGGAACTGCATGGGATGAGAT
TGAGACTTTCTTAGATATGTGTTACAGTGTCCTAA
TGCAGGCATGGATAGTGACTTGCAAGTGCATGACT
GCGCCAGACCAACCTGCTGCTTCTATTGAGAAACG
CCTGCAAAAATATCGTCAGCAAGGCAGGATCAACC
CGAGATATCTCCTGCAACCGGAGGCTCGAAGAATA
ATCCAGAATGTAATCCGAAAGGGAATGGTGGTCAG
ACATTTCCTCACCTTTGAACTGCAGCTTGCCCGAG
CACAAAGCCTTGTATCAAATAGGTATTATGCTATG
GTAGGGGATGTTGGAAAGTATATAGAGAATTGTGG
AATGGGAGGCTTCTTTTTGACACTAAAATATGCAT
TAGGAACCAGATGGCCCACACTTGCTTTAGCTGCA
TTTTCAGGAGAGCTAACAAAGCTAAAGTCCCTCAT
GGCATTATACCAGACCCTTGGTGAGCAGGCCCGAT
ATTTGGCCCTATTGGAGTCACCACATTTGATGGAT
TTTGCTGCAGCAAACTACCCACTGCTATATAGCTA
TGCTATGGGAATAGGCTATGTGTTAGATGTCAACA
TGAGGAACTACGCTTTCTCCAGATCATACATGAAT
AAGACATATTTCCAATTGGGAATGGAAACTGCAAG
AAAACAACAGGGTGCAGTTGACATGAGGATGGCAG
AAGATCTCGGTCTAACTCAAGCCGAACGCACCGAG
ATGGCAAATACACTTGCCAAATTGACCACAGCAAA
TCGAGGGGCAGACACCAGGGGAGGAGTCAACCCGT
TCTCATCTATCACTGGGACAACTCAGGTGCCCGCT
GCAGCAACAGGTGACACATTCGAGAGTTACATGGC
AGCGGATCGACTGAGGCAGAGATATGCTGATGCAG
GCACCCACGATGATGAGATGCCACCATTGGAAGAG
GAGGAAGAGGACGACACATCTGCAGGTCCACGCAC
TGGACTAACTCTTGAACAAGTGGCCTTGGACATCC
AGAACGCAGCAGTTGGAGCTCCCATCCATACAGAT
GACCTGAATGCCGCACTGGGTGATCTTGACATCTA
GACAATTCAGATCCCAATCTTAAATCGACACACCT
AATTGACCAGTTAGATGGAACTACAGTGGATTCCA
TAAGGTTCCTGCCTACCATCGGCTTTTAAGAAAAA
AATAGGCCCGGACGGGTTAGCAACAAGCGACTGCC
GATGCCAATAACACAATCCACAATCTACAATGGAT
CCCACTGATCTGAGCTTCTCCCCAGATGAGATCAA
TAAGCTCATAGAGACAGGCCTGAATACTGTGGAGT
ATTTTACTTCCCAACAAGTCACAGGAACATCCTCT
CTTGGAAAGAATACAATACCACCAGGGGTCACAGG
ACTACTAACCAATGCTGCAGAGGCAAAGATCCAAG
AGTCAACCAACCATCAGAAGGGTTCAGTTGGTGGG
GGCGCAAAACCAAAGAAACCGCGACCAAAAATTGC
CATTGTGCCAGCAGATGACAAAACGGTGCCCGGAA
AGCCGATCCCAAACCCTCTATTAGGTCTGGACTCC
ACCCCGAGCACCCAAACTGTGCTTGATCTAAGTGG
GAAAACATTACCATCAGGATCCTATAAGGGGGTTA
AACTTGCGAAATTTGGAAAAGAAAATCTGATGACA
CGGTTCATCGAGGAACCCAGAGAGAATCCTATCGC
AACCAATTCCCCCATCGATTTTAAGAGGGGCAGGG
ATACCGGCGGGTTCCATAGAAGGGAGTACTCAATC
GGATGGGTGGGAGATGAAGTCAAGGTCACTGAGTG
GTGCAATCCATCCTGTTCTCCAATCACCGCTGCAG
CAAGGCGATTTGAATGCACTTGTCACCAGTGTCCA
GTCACTTGCTCTGAATGTGAACGAGATACTTAATA
CAGTGAGAAATTTGGACTCTCGGATGAATCAACTG
GAGACAAAAGTAGATCGCATTCTCTCATCTCAGTC
TCTAATCCAGACCATCAAGAATGACATAGTTGGAC
TTAAAGCAGGGATGGCTACTTTAGAAGGAATGATT
ACAACTGTGAAAATCATGGACCCGGGAGTTCCCAG
TAATGTTACTGTGGAAGATGTACGCAAGAAACTAA
GTAACCATGCTGTTGTTGTGCCAGAATCATTCAAT
GATAGTTTCTTGACTCAATCTGAAGATGTAATTTC
ACTTGATGAGTTGGCTCGACCAACTGCAACAAGTG
TTAAGAAGATTGTCAGGAAGGTTCCTCCTCAGAAG
GATCTGACTGGATTGAAGATCACACTAGAGCAATT
GGCAAAGGATTGCATCAGCAAACCGAAGATGAGGG
AAGAGTATCTCCTCAAAATCAACCAGGCTTCCAGT
GAGGCTCAGCTAATTGACCTCAAGAAAGCAATCAT
CCGCAGTGCAATTTGATCAAGAAACACCCAATTAC
ACTACACTGGTATGACACTGTACTAACCCTGAGGG
TTTTAGAAAAAACGATTAACGATAAATAAGCCCGA
ACACTACACACCACCCGAGGCAGCCATGCCATCCA
TCAGCATCCCCGCAGACCCCACCAATCCACGTCAA
TCAATAAAAGCGTTCCCAATTGTGATCAACCGTGA
TGGGGGTGAGAAAGGTCGCTTGGTTAAACAACTAC
GCACAACCTACTTGAATGACCTAGATACTCATGAG
CCACTGGTGACATTCGTAAATACTTATGGATTCAT
CTACGAACAGGATCGGGGAAATACCATTGTCGGAG
AGGATCAACATGGGAAGAAAAGAGAGGCTGTGACT
GCTGCAATGGTTACCCTTGGATGTGGGCCTAATCT
ACCATCATTAGGGAATGTCCTGGGACAACTGAGTG
AATTCCAGGTCATTGTTAGGAAGACATCCAGCAAA
GCGGAAGAGATGGTCTTTGAAATTGTTAAGTATCC
GAGAATATTTCGGGGTCATACATTAATCCAGAAAG
GACTAGTCTGTGTCTCCGCAGAAAAATTTGTTAAG
TCACCAGGGAAAGTACAATCTGGAATGGACTATCT
CTTCATTCCGACATTTCTGTCAGTGACTTACTGTC
CAGCTGCAATCAAATTTCAGGTACCTGGCCCCATG
TTGAAAATGAGATCAAGATACACTCAGAGCTTACA
ACTTGAACTAATGATAAGAATCCTGTGTAAGCCCG
ATTCGCCACTTATGAAGGTCCATATCCCTGACAAG
GAAGGAAGAGGATGTCTTGTATCAGTATGGTTGCA
TGTATGCAATATCTTCAAATCAGGAAACAAGAATG
GCAGTGAGTGGCAGGAATACTGGATGAGAAAGTGT
GCTAACATGCAACTTGAAGTGTCGATTGCAGATAT
GTGGGGACCAACTATCATAATTCATGCCAGAGGTC
ACATTCCCAAAAGTGCTAAGTTGTTTTTTGGAAAG
GGTGGATGGAGCTGCCATCCACTTCACGAAGTTGT
TCCAAGTGTCACTAAAACACTATGGTCCGTGGGCT
GTGAGATTACAAAGGCGAAGGCAATAATACAAGAG
AGTAGCATCTCTCTTCTCGTGGAGACTACTGACAT
CATAAGTCCAAAAGTCAAAATTTCATCTAAGCATC
GCCGCTTTGGGAAATCAAATTGGGGTCTGTTCAAG
AAAACCAAATCACTGCCCAACCTGACGGAGCTGGA
ATGACTGACCCCCAATCGAGACTACACCACCTCAA
ACTATAGGTGGGTGGTACCTCAGTGATTAATCTCG
TAAGCACTGATCGTAGGCTACAACACACTAATATT
ATCCAGATTAGAGAGCTTAATTAGCTCTGTATTAA
TAATAACACTACTATTCCAATAACTGGAATCACCA
GCTTGATTTATCTCCAAAATGATTCAAAGAAAACA
AATCATATTAAGACTATCCTAAGCACGAACCCATA
TCGTCCTTCAAATCATGGGTACTATAATTCAATTT
CTGGTGGTCTCCTGTCTATTGGCAGGAGCAGGCAG
TCTTGATCCAGCAGCCCTCATGCAAATCGGTGTCA
TTCCAACAAATGTCCGGCAACTTATGTATTATACT
GAGGCCTCATCAGCATTCATTGTTGTGAAGTTAAT
GCCTACAATTGACTCGCCGATTAGTGGATGTAATA
TAACATCAATTTCAAGCTATAATGCAACAGTGACA
AAACTCCTACAGCCGATCGGTGAGAATTTGGAGAC
GATTAGGAACCAGTTGATTCCAACTCGGAGGAGAC
GCCGGTTTGCAGGGGTGGTGATTGGATTAGCTGCA
TTAGGAGTAGCTACTGCCGCACAGGTCACTGCCGC
AGTAGCACTAGTAAAGGCAAATGAAAATGCTGCGG
CTATACTCAATCTCAAAAATGCAATCCAAAAAACA
AATGCAGCAGTTGCAGATGTGGTCCAGGCCACACA
ATCACTAGGAACaGCAGTTCAAGCAGTTCAAGATC
ACATAAACAGTGTGGTAAGTCCAGCAATTACAGCA
GCCAATTGTAAAGCCCAAGATGCTATCATTGGCTC
AATCCTCAATCTCTATTTGACCGAGTTGACAACTA
TCTTCCACAATCAAATTACAAACCCTGCATTGAGT
CCTATTACAATTCAAGCTTTAAGGATCCTACTAGG
GAGTACCTTGCCGACTGTGGTCGAAAAATCTTTCA
ATACCCAGATAAGTGCGGCTGAGCTTCTCTCATCA
GGGTTATTGACAGGCCAGATTGTGGGATTAGATTT
GACCTATATGCAGATGGTCATAAAAATTGAGCTGC
CAACTTTAACTGTACAACCTGCAACCCAGATCATA
GATCTGGCCACCATTTCTGCATTCATTAACAATCA
AGAAGTCATGGCCCAATTACCAACACGTGTTATTG
TGACTGGCAGCTTGATCCAAGCCTATCCCGCATCG
CAATGCACTATTACACCCAACACTGTGTACTGTAG
GTATAATGATGCCCAAGTACTCTCAGATGATACGA
TGGCTTGCCTCCAAGGTAACTTGACAAGATGCACC
TTCTCTCCAGTGGTTGGGAGCTTTCTCACTCGATT
CGTGCTGTTCGATGGAATAGTTTATGCAAATTGCA
GGTCGATGTTGTGCAAGTGCATGCAGCCTGCTGCT
GTGATCCTACAGCCGAGTTCATCCCCTGTAACTGT
CATTGACATGTACAAATGTGTGAGTCTGCAGCTTG
ACAATCTCAGATTCACCATCACTCAATTGGCCAAT
GTAACCTACAATAGCACCATCAAGCTTGAAACATC
CCAGATCTTGCCTATTGATCCGTTGGATATATCCC
AGAATCTAGCTGCGGTGAATAAGAGTCTAAGTGAT
GCACTACAACACTTAGCACAAAGTGACACATACCT
TTCTGCAATCACATCAGCTACGACTACAAGTGTAT
TATCCATAATAGCAATCTGTCTTGGATCGTTAGGT
TTAATATTAATAATCTTGCTCAGTGTAGTTGTGTG
GAAGTTATTGACCATTGTCGCTGCTAATCGAAATA
GAATGGAGAATTTTGTTTATCATAATTCAGCATTC
CACCACTCACGATCTGATCTCAGTGAGAAAAATCA
ACCTGCAACTCTTGGAACAAGATAAGACAGTCATC
CATTAGTAATTTTTAAGAAAAAAACGATAGGACCG
AAACTAGTATTGAAAGAACCGTCTCGGTCAATCTA
GGTAATCGAGCTGcTACCGTCTCGGAAAGCTCAAA
TCATGCTGCCTGATCCGGAAGATCCGGAAAGCAAA
AAAGCTACAAGGAGAACAGGAAACCTAATTATCTG
CTTCCTATTCATCTTCTTTCCGTTTGTAAACTTCA
TTGTTCCAACTCTAAGACACTTGCTGTCCTAACAC
CTGCTATAGGCTATCCACTGCATCATCTCTTTTTA
AGAAAAAAATAGGCCCGGACGGGTTAGCAACAAGC
GACTGCCGGTGCCAACAACACAATCCACAATCTAC
AGTCGACGCGGCCGCCACCatgttcgtgttcctgg
tgctgctgccactggtcagcagccagtgcgtgaat
ttcactacccggacccagctgcctcctgcttatac
aaacagcttcaccaggggagtgtactaccccgata
aggtgttccgcagctctgtgctgcacagcacccag
gacttgtttcttcccttcttcagcaacgtgacctg
gttccacgccatccacgtgtccggcaccaacggca
ccaaaagattcgccaatcctgtgctgcctttcaac
gatggcgtgtactttgcctctacagagaagtctaa
catcatcagaggctggatcttcggcacaaccctgg
attctaaaacccagagcctgctgatcgtgaacaat
gccaccaatgtagtgatcaaggtttgtgagttcca
gttctgcaacgacccctttctgggcgtttactacc
acaagaacaacaaaagctggatggaaagcgagttc
agggtgtactctagcgccaacaactgtaccttcga
gtacgtgagccagccttttctgatggacctggagg
gcaagcagggcaacttcaaaaacctgcgggagttc
gtgtttaagaacatcgacggatacttcaagattta
ctccaagcacaccccaattaacttggttagaggac
tgccccaaggcttcagcgccctggaacccctggtg
gacctgcctatcggcatcaacatcacaagattcca
aaccctgcatagaagctatctgacaccaggcgact
ctagctctggatggacagccggcgccgctgcttat
tacgtgggctacctgcagcctagaaccttcctcct
gaaatacaacgagaacggaaccatcaccgacgccg
tggactgcgccctggatcctctgagcgagacaaag
tgcaccctgaagtccttcaccgtcgaaaagggcat
ctaccaaacaagcaacttcagagtgcagcctactg
agagcatcgtgcggtttccaaacatcaccaacctg
tgtccttttggcgaggtgttcaacgccacccggtt
cgcttccgtgtacgcctggaatagaaagcggatca
gcaactgcgtggccgactacagcgtgctgtataat
agcgcctcattttctacttttaaatgttacggcgt
gtctcctacaaagctgaacgatctgtgcttcacaa
atgtgtacgccgatagcttcgtgattagaggcgac
gaggtgcggcagatcgcccctggccagaccggcaa
catcgctgactacaactacaagctgcctgacgact
tcaccggctgtgtgatcgcatggaacagcaataac
ctggactctaaggtgggcggaaactacaattacct
gtacagactgttcagaaagagcaacctgaagccct
tcgagagagatatctccacagaaatctatcaggcc
ggatctacaccttgtaatggcgtgaagggcttcaa
ctgctacttccctctgcaatcctacggctttcagc
ccacatacggcgtgggctaccagccataccgggtc
gtggtgctgagcttcgagctgctgcacgcccctgc
cacagtgtgcggccctaagaagagcaccaatctgg
taaagaacaagtgcgtcaacttcaatttcaacggc
ctgaccgggaccggagtgctgaccgaaagcaacaa
gaaattcctgccgttccagcagttcggcagagata
tcgccgacaccaccgatgccgtgcgggacccccag
acacttgaaatcctggacatcaccccttgcagttt
tggcggcgtgtccgtgatcacacctggaacaaaca
ccagcaaccaggtggccgtgctgtaccagggcgtc
aactgcaccgaggtccccgtggccatccacgccga
tcagctgacacctacatggcgggtgtactctaccg
gcagcaacgtgttccaaaccagagccggctgcctg
atcggcgctgagcacgtgaacaacagctacgagtg
cgacattcctatcggtgctggcatctgcgcctctt
accagacacagacaaacagccccagaagagcgaga
agcgtggctagtcagagcatcatcgcctacaccat
gagcctgggcgtggagaacagcgtggcctactcta
acaatagcatcgccatcccaaccaacttcacaatc
tctgtgaccacagagatcctgcccgtgtccatgac
caagacatctgtggactgcacaatgtatatctgtg
gcgattctaccgagtgcagcaacctgctgctccag
tacggcagcttttgtacccagctgaatagagccct
gacaggcatcgccgtggagcaggataagaacacac
aggaggtgttcgcccaggtgaagcaaatctacaag
accccccctatcaaggactttggcggcttcaattt
ttcccagatcctgcctgatccatccaagccttcta
agcggagctttatcgaggacctgctgttcaacaag
gtgaccctggccgatgccggcttcatcaagcagta
tggcgattgcctgggcgacatcgcagccagggacc
tgatctgcgcccagaagtttaatggcctgaccgtg
ctgccacccctgctgacagatgagatgatcgcaca
gtacacaagcgccctgctggccggcaccatcacat
ccggatggaccttcggcgcaggagccgccctccag
atcccctttgccatgcagatggcctataggttcaa
cggcatcggcgtgacccagaatgtgctgtacgaga
accagaagctgatcgccaatcagtttaactccgcc
atcggcaagatccaggacagcctgtcctctacagc
cagcgccctgggcaagctccaggatgtggtgaatc
agaacgcccaggccctgaataccctggtgaagcag
ctgagcagcaacttcggcgccatctctagcgtgct
gaatgacatcctgagccggctggacaaggtggagg
cagaggtgcagatcgaccggctgatcaccggccgg
ctccagagcctccagacctatgtgacacagcagct
gatcagggccgccgagatcagggccagcgccaatc
tggcagcaaccaagatgtccgagtgcgtgctgggc
cagtctaagagagtggacttttgtggcaagggcta
tcacctgatgtccttccctcagtctgccccacacg
gcgtggtgtttctgcacgtgacctacgtgcccgcc
caggagaagaacttcaccacagcccctgccatctg
ccacgatggcaaggcccactttccaagggagggcg
tgttcgtgtccaacggcacccactggtttgtgaca
cagcgcaatttctacgagccccagatcatcaccac
agacaacaccttcgtgagcggcaactgtgacgtgg
tcatcggcatcgtgaacaataccgtgtatgatcca
ctccagcccgagctggacagctttaaggaggagct
ggataagtatttcaagaatcacacctcccctgacg
tggatctgggcgacatcagcggcatcaatgcctcc
gtggtgaacatccagaaggagatcgaccgcctgaa
cgaggtggctaagaatctgaacgagagcctgatcg
acctccaggagctgggcaagtatgagcagtacatc
aagtggccctggtacatctggctgggcttcatcgc
cggcctgatcgccatcgtgatggtgaccatcatgc
tgtgctgtatgacatcctgctgttcttgcctgaag
ggctgctgtagctgtggctcctgctgtAAGTTATT
GACCATTGTCGTTGCTAATCGAAATAGAATGGAGA
ATTTTGTTTATCATAAATGAACGCGTGCTAGCCTC
CTGCCATACTTCCTACTCACATCATATCTATTTTA
AAGAAAAAATAGGCCCGAACACTAATCGTGCCGGC
AGTGCCACTGCACACACAACACTACACATACAATA
CACTACAATGGTTGCAGAAGATGCCCCTGTTAGGG
GCACTTGCCGAGTATTATTTCGAACAACAACTTTA
ATTTTTCTATGCACACTATTAGCATTAAGCATCTC
TATCCTTTATGAGAGTTTAATAACCCAAAAGCAAA
TCATGAGCCAAGCAGGCTCAACTGGATCTAATTCT
AGATTAGGAAGTATCACTGATCTTCTTAATAATAT
TCTCTCTGTCGCAAATCAGATTATATATAACTCTG
CAGTCGCTCTACCTCTACAATTGGACACTCTTGAA
TCAACACTCCTTACAGCCATTAAGTCTCTTCAAAC
AAGTGACAAGCTAGAACAGAACTGCTCGTGGGGTG
CTGCACTGATTAATGATAATAGATACATTAATGGC
ATCAATCAGTTCTATTTCTCAATTGCTGAGGGTCG
CAAGCTGACACTTGGCCCACTTCTTAATATACCTA
GTTTCATTCCAACTGCCACGACACCAGAGGGCTGC
ACCAGGATCCCATCATTCTCGCTCACCAAGACACA
CTGGTGTTATACACACAATGTTATCCTGAATGGAT
GCCAGGATCATGTATCCTCAAATCAATTTGTTTCC
ATGGGAATCATTGAACCCACTTCTGCCGGGTTTCC
ATCCTTTCGAACCCTAAAGACTCTATATCTCAGCG
ATGGGGTCAATCGTAAGAGCTGCTCTATCAGTACA
GTTCCGGGGGGTTGTATGATGTACTGTTTCGTCTC
TACTCAACCAGAGAGGGATGACTACTTTTCTACCG
CTCCTCCAGAACAACGAATTATTATAATGTACTAT
AATGATACAATCGTGGAGCGCATAATTAATCCACC
CGGGGTACTAGATGTATGGGCAACATTGAACCCAG
GAACAGGAAGCGGGGTATATTATTTAGGTTGGGTA
CTCTTTCCAATATATGGCGGCGTGATTAAAGATAC
GAGTTTATGGAATAATCAAGCAAATAAATACTTTA
TCCCCCAGATGGTTGCTGCTCTCTGCTCACAAAAC
CAGGCAACTCAAGTCCAAAATGCTAAGTCATCATA
CTATAGCAGCTGGTTTGGCAATCGAATGATTCAGT
CTGGGATCCTGGCATGTCCTCTTCAACAGGATCTA
ACCAATGAGTGTTTAGTTCTGCCCTTTTCTAATGA
TCAGGTGCTTATGGGTGCTGAAGGGAGATTATACA
TGTATGGTGACTCGGTGTATTACTACCAAAGAAGC
AATAGTTGGTGGCCTATGACCATGCTGTATAAGGT
AACCATAACATTCACTAATGGTCAGCCATCTGCTA
TATCAGCTCAGAATGTGCCCACACAGCAGGTCCCT
AGACCTGGGACAGGAAGCTGCTCTGCAACAAATAG
ATGTCCCGGTTTTTGCTTGAAAGGAGTGTATGCTG
ATGCCTGGTTACTGACCAACCCTTCGTCTACCAGT
ACATTTGGATCAGAAGCAACCTTCACTGGTTCTTA
TCTCAACGCAGCAACTCAGCGTATCAATCCGACGA
TGTATATCGCGAACAACACACAGATCATAAGCTCA
CAGCAATTTGGATCAAGCGGTCAAGAAGCAGCATA
TGGCCACACAACTTGTTTTAGGGACACAGGCTCTG
TTATGGTATACTGTATCTATATTATTGAATTGTCC
TCATCTCTCTTAGGACAATTTCAGATTGTCCCATT
TATCCGTCAGGTGACACTATCCTAAAGGCAGAAGC
CTCCAGGTCTGACCCAGCCAATCAAAGCATTATAC
CAGACCATGGAATGCATACCAAACATTATTGACAC
TAATGACACACAAAATTGGTTTTAAGAAAAACCAA
GAGAACAATAGGCCAGAATGGCTGGGTCTCGGGAG
ATATTACTCCCTGAAGTCCATCTCAATTCACCAAT
TGTAAAGCATAAGCTATACTATTACATTCTACTTG
GAAACCTCCCAAATGAGATCGACATTGACGATTTA
GGTCCATTACATAATCAAAATTGGAATCAAATAGC
ACATGAAGAGTCTAACTTAGCCCAACGCTTGGTAA
ATGTAAGAAATTTTCTAATTACCCACATCCCTGAT
CTTAGAAAGGGCCATTGGCAAGAGTATGTCAATGT
AATACTGTGGCCGCGAATTCTTCCCTTGATCCCGG
ATTTTAAAATCAATGACCAATTGCCTCTACTCAAA
AATTGGGACAAGTTAGTTAAAGAATCATGTTCAGT
AATCAATGCGGGTACTTCCCAGTGCATTCAGAATC
TCAGCTATGGACTGACAGGTCGTGGGAACCTCTTT
ACACGATCACGTGAACTCTCTGGTGACCGCAGGGA
TATTGATCTTAAGACGGTTGTGGCAGCATGGCATG
ACTCAGACTGGAAAAGAATAAGTGATTTTTGGATT
ATGATCAAATTCCAGATGAGACAATTAATTGTTAG
GCAAACAGATCATAATGATCCTGATTTAATCACGT
ATATCGAAAATAGAGAAGGCATAATCATCATAACC
CCTGAACTGGTAGCATTATTTAACACTGAGAATCA
TACACTAACATACATGACCTTTGAAATTGTACTGA
TGGTTTCAGATATGTACGAAGGTCGTCACAACATT
TTATCACTATGCACAGTTAGCACTTACCTGAATCC
TCTGAAGAAAAGAATAACATATTTATTGAGCCTTG
TAGATAACTTAGCTTTTCAGATAGGTGATGCTGTA
TATAACATAATTGCTTTGCTAGAATCCTTTGTATA
TGCACAGTTGCAAATGTCAGATCCCATCCCAGAAC
TCAGAGGACAATTCCATGCATTCGTATGTTCTGAG
ATTCTTGATGCACTAAGGGGAACTAATAGTTTCAC
CCAGGATGAATCAAGAACTGTGACAACCAATTTGA
TATCCCCATTCCAAGATCTGACCCCAGATCTTACG
GCTGAATTGCTCTGTATAATGAGGCTTTGGGGACA
CCCCATGCTCACCGCCAGTCAAGCTGCGGGAAAGG
TACGCGAGTCCATGTGTGCTGGAAAAGTATTAGAC
TTTCCCACCATTATGAAAACACTAGCCTTTTTCCA
TACTATTCTGATCAATGGATACAGGAGGAAGCATC
ATGGAGTATGGCCACCCTTAAACTTACCGGGTAAT
GCTTCAAAGGGTCTCACGGAACTTATGAATGACAA
TACTGAGATAAGCTATGAATTCACACTTAAGCATT
GGAAGGAAATCTCTCTTATAAAATTCAAGAAATGT
TTTGATGCAGACGCAGGTGAGGAACTCAGTATATT
TATGAAAGATAAAGCAATTAGTGCCCCAAAACAAG
ATTGGATGAGTGTGTTTAGAAGAAGCCTAATCAAA
CAGCGCCATCAGCATCATCAGGTCCCCCTACCAAA
TCCATTCAATCGACGGCTATTGCTAAACTTTCTCG
GAGATGACAAATTCGACCCGAATGTGGAGCTACAG
TATGTAACATCAGGTGAGTATCTACATGATGACAC
GTTTTGTGCATCATATTCACTAAAAGAGAAGGAAA
TTAAACCTGATGGTCGAATTTTTGCAAAGTTGACT
AAGAGAATGAGATCATGTCAAGTTATAGCAGAATC
TCTTTTAGCGAACCATGCTGGGAAGTTAATGAAAG
AGAATGGTGTTGTGATGAATCAGCTATCATTAACA
AAATCACTATTAACAATGAGTCAGATTGGAATAAT
ATCCGAGAAAGCTAGAAAATCGACTCGAGATAACA
TAAATCAACCTGGTTTCCAGAATATCCAGAGAAAT
AAATCACATCACTCCAAGCAAGTCAATCAGCGAGA
TCCAAGTGATGACTTTGAATTGGCAGCATCTTTTT
TAACTACTGATCTCAAAAAATATTGTTTACAATGG
AGGTACCAGACAATTATCCCATTTGCTCAATCATT
AAACAGAATGTATGGTTATCCTCATCTCTTTGAGT
GGATTCACTTACGGCTAATGCGTAGTACACTTTAC
GTGGGGGATCCCTTCAACCCACCAGCAGATACCAG
TCAATTTGATCTAGATAAAGTAATTAATGGAGATA
TCTTCATTGTATCACCCAGAGGTGGAATTGAAGGG
CTATGTCAAAAGGCTTGGACAATGATATCTATCTC
TGTGATAATTCTATCTGCCACAGAGTCTGGCACAC
GAGTAATGAGTATGGTGCAGGGAGATAATCAAGCA
ATTGCTGTCACCACACGAGTACCAAGGAGCCTGCC
GACTCTTGAGAAAAAGACTATTGCTTTTAGATCTT
GTAATCTATTCTTTGAGAGGTTAAAATGTAATAAT
TTTGGATTAGGTCACCATTTGAAAGAACAAGAGAC
TATCATTAGTTCTCACTTCTTTGTTTATAGCAAGA
GAATATTCTATCAGGGGAGGATTCTAACGCAAGCC
TTAAAAAATGCTAGTAAGCTCTGCTTGACAGCTGA
TGTCCTAGGAGAATGTACCCAATCATCATGTTCTA
ATCTTGCAACTACTGTCATGAGGTTAACTGAGAAT
GGTGTTGAAAAAGATATCTGTTTCTACTTGAATAT
CTATATGACCATCAAACAGCTCTCCTATGATATCA
TCTTCCCTCAAGTGTCAATTCCTGGAGATCAGATC
ACATTAGAATACATAAATAATCCACACCTGGTATC
ACGATTGGCTCTTCTGCCATCCCAGCTAGGAGGTC
TAAACTACCTGTCATGCAGTAGGCTGTTCAATCGA
AACATAGGCGACCCGGTGGTTTCCGCAGTTGCAGA
TCTTAAGAGATTAATTAAATCAGGATGTATGGATT
ACTGGATCCTTTATAACTTATTAGGGAGAAAACCG
GGAAACGGCTCATGGGCTACTTTAGCAGCTGACCC
GTACTCAATCAATATAGAGTATCAATACCCCCCAA
CTACAGCTCTTAAGAGGCACACCCAACAAGCTCTG
ATGGAACTCAGTACGAATCCAATGTTACGTGGCAT
ATTCTCTGACAATGCACAGGCAGAAGAAAATAATC
TTGCTAGATTTCTCCTGGATAGGGAGGTGATCTTT
CCGCGTGTAGCTCACATCATCATTGAGCAAACCAG
TGTCGGGAGGAGAAAACAGATTCAAGGATATTTGG
ATTCAACTAGATCGATAATGAGTAAATCACTAGAA
ATTAAGCCCTTGTCCAATAGGAAGCTTAATGAAAT
ACTGGATTACAACATCAATTACCTAGCTTACAATT
TGGCATTACTCAAGAATGCTATTGAACCTCCGACT
TATTTGAAAGCAATGACTCTTGAAACATGTAGCAT
CGACATTGCAAGGAGCCTCCGGAAGCTCTCCTGGG
CCCCACTCTTGGGTGGGAGAAATCTTGAAGGATTA
GAGACGCCAGATCCCATTGAAATTACTGCAGGAGC
ATTAATTGTTGGATCGGGCTACTGTGAACAGTGTG
CTGCAGGAGACAATCGATTCACATGGTTTTTCTTG
CCATCTGGTATCGAGATAGGAGGGGATCCCCGTGA
TAATCCTCCTATCCGTGTACCGTACATTGGCTCCA
GGACTGATGAGAGGAGGGTAGCCTCAATGGCATAC
ATCAGGGGTGCCTCGAGTAGCCTAAAAGCAGTTCT
TAGACTGGCGGGAGTGTACATCTGGGCATTCGGAG
ATACTCTGGAGAATTGGATAGATGCACTGGATTTG
TCTCACACTAGAGTTAACATCACACTTGAACAGCT
GCAATCCCTCACCCCACTTCCAACCTCTGCCAATC
TAACCCATCGGTTGGATGATGGCACAACTACCCTA
AAGTTTACTCCTGCGAGCTCTTACACCTTTTCAAG
TTTCACTCATATATCAAATGATGAGCAATACCTGA
CAATTAATGACAAAACTGCAGATTCAAATATAATC
TACCAACAGTTAATGATCACTGGACTCGGAATTTT
AGAAACATGGAATAATCCCCCAATCAATAGAACAT
TCGAAGAATCTACCCTACATTTGCACACTGGTGCA
TCATGTTGTGTCCGACCTGTGGACTCCTGCATCAT
CTCAGAAGCATTAACAGTCAAGCCACATATTACAG
TACCGTACAGCAATAAATTTGTATTTGATGAAGAC
CCGCTATCTGAATATGAGACTGCAAAACTGGAATC
GTTATCATTTCAAGCCCAATTAGGCAACATTGATG
CTGTAGATATGACAGGTAAATTAACATTATTGTCC
CAATTCACTGCAAGGCAGATTATTAATGCAATCAC
TGGACTCGATGAGTCTGTCTCTCTTACTAATGATG
CCATTGTTGCATCAGACTATGTCTCCAATTGGATT
AGTGAATGCATGTATACCAAATTAGATGAATTATT
TATGTATTGTGGGTGGGAACTACTATTGGAACTAT
CCTATCAAATGTATTATCTGAGGGTAGTTGGGTGG
AGTAATATAGTGGATTATTCTTACATGATCTTGAG
AAGAATCCCGGGTGCAGCATTAAACAATCTGGCAT
CTACATTAAGTCATCCAAAACTTTTCCGACGAGCT
ATCAACCTAGATATAGTTGCCCCCTTAAATGCTCC
TCATTTTGCATCTCTGGACTACATCAAGATGAGTG
TGGATGCAATACTCTGGGGCTGTAAAAGAGTCATC
AATGTGCTCTCCAATGGAGGGGACTTAGAATTAGT
TGTGACATCTGAAGATAGCCTTATTCTCAGTGACC
GATCCATGAATCTCATTGCAAGGAAATTAACTTTA
TTATCACTGATTCACCATAATGGTTTGGAACTACC
AAAGATTAAGGGGTTCTCTCCTGATGAGAAGTGTT
TCGCTTTGACAGAATTTTTGAGGAAAGTGGTGAAC
TCAGGGTTGAGTTCAATAGAGAACCTATCAAATTT
TATGTACAATGTGGAGAACCCACGGCTTGCAGCAT
TCGCCAGCAACAATTACTACCTGACCAGAAAATTA
TTGAATTCAATACGAGATACTGAGTCGGGTCAAGT
AGCAGTCACCTCATATTATGAATCATTAGAATATA
TTGATAGTCTTAAGCTAACCCCACATGTGCCTGGT
ACCTCATGCATTGAGGATGATAGTCTATGTACAAA
TGATTACATAATCTGGATCATAGAGTCTAATGCAA
ACTTGGAGAAGTATCCAATTCCAAATAGCCCTGAG
GATGATTCCAATTTCCATAACTTTAAGTTGAATGC
TCCATCGCACCATACCTTACGCCCATTAGGGTTGT
CATCAACTGCTTGGTATAAGGGTATAAGCTGTTGC
AGGTACCTTGAGCGATTAAAGCTACCACAAGGTGA
TCATTTATATATTGCAGAAGGTAGTGGTGCCAGTA
TGACAATCATAGAATACCTATTCCCAGGAAGAAAG
ATATATTACAATTCTTTATTTAGTAGTGGTGACAA
TCCCCCACAAAGAAATTATGCACCAATGCCTACTC
AGTTCATTGAGAGTGTCCCATACAAGCTCTGGCAA
GCACACACAGATCAATATCCCGAGATTTTTGAGGA
CTTCATCCCTCTATGGAACGGAAATGCCGCCATGA
CTGACATAGGAATGACAGCTTGTGTAGAATTCATC
ATCAATCGAGTCGGCCCAAGGACTTGCAGTTTAGT
ACATGTAGATTTGGAATCAAGTGCAAGCTTAAATC
AACAATGCCTGTCAAAGCCGATAATTAATGCTATC
ATCACTGCTACATGATCACTTTCTTATGGTGCTAC
TTTGAGAGAATCACTGTTCTTAGGAGCACATATTC
TGGTCCAGCTAATCATGAGGTTTATTTAATTTGTA
TCCTTGCCAACAACTTTGCATTCCAGACTGTCTCG
CAGGCAACAGGAATGGCGATGACTTTAACCGATCA
AGGGTTTACTTTGATATCACCTGAAAGAATAAATC
AGTATTGGGATGGTCACTTGAAGCAAGAACGTATC
GTAGCAGAAGCAATTGATAAGGTGGTTCTAGGAGA
AGATGCTCTATTCAATTCGAGTGATAATGAATTAA
TTCTCAAATGTGGAGGGACACCAAATGCACGGAAT
CTTATCGATATCGAGCCAGTCGCAACTTTCATAGA
ATTTGAACAACTGATCTGCACAATGTTGACAACCC
ACTTGAAGGAAATAATTGATATAACAAGGTCTGGA
ACCCAGGATTATGAAAGTTTATTACTCACTCCTTA
CAATTTAGGTCTTCTTGGTAAAATCAGTACGATAG
TGAGATTATTAACAGAAAGGATTCTAAATCATACT
ATCAGGAATTGGTTGATCCTCCCACCTTCGCTCCG
GATGATCGTGAAGCAGGACTTGGAATTCGGCATAT
TCAGGATTACTTCCATCCTCAATTCTGATCGGTTC
CTGAAGCTTTCTCCAAATAGGAAATACTTGATTAC
ACAATTAACTGCAGGCTACATTAGGAAATTGATTG
AGGGGGATTGTAATATCGATCTAACCAGACCTATC
CAAAAACAAATCTGGAAAGCATTAGGTTGTGTAGT
CTATTGTCACGATCCAGTAGATCAAAGGGAATCAA
CAGAGTTTATTGATATAAATATTAATGAAGAAATA
GACCGCGGGATCGATGGCGAGGAAATCTAAATATA
TCAAGAATCAGAATTAGTTTAAGAAAAAAGAAGAG
GATTAATCTTGGTTTTCCCCTTGGT.

3. CVL48

The nucleic acid sequence for CVL48 is:

(SEQ ID NO: 63)
ACCAGGGGGAAAACGAAGTGGTGACTCAAATCATC
GAAGACCCTCGAGATTACATAGGTCCGGAACCTAT
GGCCTTCGTGACCGACCTCGAGTCAGAGTAGTTCA
ATAAGGACCTATCAAGTTTGGGCAATTTTTCGTCC
CCGACACAAAAATGTCATCCGTGCTTAAAGCATAT
GAGCGATTCACACTCACTCAAGAACTGCAAGATCA
GAGTGAGGAAGGTACAATCCCACCTACAACACTAA
AACCGGTAATCAGGGTATTTATACTAACCTCTAAT
AACCCAGAGCTAAGATCCCGGCTTCTTCTATTCTG
CCTACGGATTGTTCTCAGTAATGGTGCAAGGGATT
CCCATCGCTTTGGAGCATTACTTACAATGTTTTCG
CTACCATCAGCCACAATGCTCAATCATGTCAAATT
AGCTGACCAGTCACCAGAAGCTGATATCGAAAGGG
TAGAGATCGATGGCTTTGAGGAGGGATCATTCCGC
TTAATCCCCAATGCTCGTTCAGGTATGAGCCGTGG
AGAGATCAATGCCTATGCTGCACTTGCAGAAGATC
TACCTGACACACTAAACCATGCAACACCTTTTGTT
GATTCCGAAGTCGAGGGAACTGCATGGGATGAGAT
TGAGACTTTCTTAGATATGTGTTACAGTGTCCTAA
TGCAGGCATGGATAGTGACTTGCAAGTGCATGACT
GCGCCAGACCAACCTGCTGCTTCTATTGAGAAACG
CCTGCAAAAATATCGTCAGCAAGGCAGGATCAACC
CGAGATATCTCCTGCAACCGGAGGCTCGAAGAATA
ATCCAGAATGTAATCCGAAAGGGAATGGTGGTCAG
ACATTTCCTCACCTTTGAACTGCAGCTTGCCCGAG
CACAAAGCCTTGTATCAAATAGGTATTATGCTATG
GTAGGGGATGTTGGAAAGTATATAGAGAATTGTGG
AATGGGAGGCTTCTTTTTGACACTAAAATATGCAT
TAGGAACCAGATGGCCCACACTTGCTTTAGCTGCA
TTTTCAGGAGAGCTAACAAAGCTAAAGTCCCTCAT
GGCATTATACCAGACCCTTGGTGAGCAGGCCCGAT
ATTTGGCCCTATTGGAGTCACCACATTTGATGGAT
TTTGCTGCAGCAAACTACCCACTGCTATATAGCTA
TGCTATGGGAATAGGCTATGTGTTAGATGTCAACA
TGAGGAACTACGCTTTCTCCAGATCATACATGAAT
AAGACATATTTCCAATTGGGAATGGAAACTGCAAG
AAAACAACAGGGTGCAGTTGACATGAGGATGGCAG
AAGATCTCGGTCTAACTCAAGCCGAACGCACCGAG
ATGGCAAATACACTTGCCAAATTGACCACAGCAAA
TCGAGGGGCAGACACCAGGGGAGGAGTCAACCCGT
TCTCATCTATCACTGGGACAACTCAGGTGCCCGCT
GCAGCAACAGGTGACACATTCGAGAGTTACATGGC
AGCGGATCGACTGAGGCAGAGATATGCTGATGCAG
GCACCCACGATGATGAGATGCCACCATTGGAAGAG
GAGGAAGAGGACGACACATCTGCAGGTCCACGCAC
TGGACTAACTCTTGAACAAGTGGCCTTGGACATCC
AGAACGCAGCAGTTGGAGCTCCCATCCATACAGAT
GACCTGAATGCCGCACTGGGTGATCTTGACATCTA
GACAATTCAGATCCCAATCTTAAATCGACACACCT
AATTGACCAGTTAGATGGAACTACAGTGGATTCCA
TAAGGTTCCTGCCTACCATCGGCTTTTAAGAAAAA
AATAGGCCCGGACGGGTTAGCAACAAGCGACTGCC
GATGCCAATAACACAATCCACAATCTACAATGGAT
CCCACTGATCTGAGCTTCTCCCCAGATGAGATCAA
TAAGCTCATAGAGACAGGCCTGAATACTGTGGAGT
ATTTTACTTCCCAACAAGTCACAGGAACATCCTCT
CTTGGAAAGAATACAATACCACCAGGGGTCACAGG
ACTACTAACCAATGCTGCAGAGGCAAAGATCCAAG
AGTCAACCAACCATCAGAAGGGTTCAGTTGGTGGG
GGCGCAAAACCAAAGAAACCGCGACCAAAAATTGC
CATTGTGCCAGCAGATGACAAAACGGTGCCCGGAA
AGCCGATCCCAAACCCTCTATTAGGTCTGGACTCC
ACCCCGAGCACCCAAACTGTGCTTGATCTAAGTGG
GAAAACATTACCATCAGGATCCTATAAGGGGGTTA
AACTTGCGAAATTTGGAAAAGAAAATCTGATGACA
CGGTTCATCGAGGAACCCAGAGAGAATCCTATCGC
AACCAATTCCCCCATCGATTTTAAGAGGGGCAGGG
ATACCGGCGGGTTCCATAGAAGGGAGTACTCAATC
GGATGGGTGGGAGATGAAGTCAAGGTCACTGAGTG
GTGCAATCCATCCTGTTCTCCAATCACCGCTGCAG
CAAGGCGATTTGAATGCACTTGTCACCAGTGTCCA
GTCACTTGCTCTGAATGTGAACGAGATACTTAATA
CAGTGAGAAATTTGGACTCTCGGATGAATCAACTG
GAGACAAAAGTAGATCGCATTCTCTCATCTCAGTC
TCTAATCCAGACCATCAAGAATGACATAGTTGGAC
TTAAAGCAGGGATGGCTACTTTAGAAGGAATGATT
ACAACTGTGAAAATCATGGACCCGGGAGTTCCCAG
TAATGTTACTGTGGAAGATGTACGCAAGAAACTAA
GTAACCATGCTGTTGTTGTGCCAGAATCATTCAAT
GATAGTTTCTTGACTCAATCTGAAGATGTAATTTC
ACTTGATGAGTTGGCTCGACCAACTGCAACAAGTG
TTAAGAAGATTGTCAGGAAGGTTCCTCCTCAGAAG
GATCTGACTGGATTGAAGATCACACTAGAGCAATT
GGCAAAGGATTGCATCAGCAAACCGAAGATGAGGG
AAGAGTATCTCCTCAAAATCAACCAGGCTTCCAGT
GAGGCTCAGCTAATTGACCTCAAGAAAGCAATCAT
CCGCAGTGCAATTTGATCAAGAAACACCCAATTAC
ACTACACTGGTATGACACTGTACTAACCCTGAGGG
TTTTAGAAAAAACGATTAACGATAAATAAGCCCGA
ACACTACACACCACCCGAGGCAGCCATGCCATCCA
TCAGCATCCCCGCAGACCCCACCAATCCACGTCAA
TCAATAAAAGCGTTCCCAATTGTGATCAACCGTGA
TGGGGGTGAGAAAGGTCGCTTGGTTAAACAACTAC
GCACAACCTACTTGAATGACCTAGATACTCATGAG
CCACTGGTGACATTCGTAAATACTTATGGATTCAT
CTACGAACAGGATCGGGGAAATACCATTGTCGGAG
AGGATCAACATGGGAAGAAAAGAGAGGCTGTGACT
GCTGCAATGGTTACCCTTGGATGTGGGCCTAATCT
ACCATCATTAGGGAATGTCCTGGGACAACTGAGTG
AATTCCAGGTCATTGTTAGGAAGACATCCAGCAAA
GCGGAAGAGATGGTCTTTGAAATTGTTAAGTATCC
GAGAATATTTCGGGGTCATACATTAATCCAGAAAG
GACTAGTCTGTGTCTCCGCAGAAAAATTTGTTAAG
TCACCAGGGAAAGTACAATCTGGAATGGACTATCT
CTTCATTCCGACATTTCTGTCAGTGACTTACTGTC
CAGCTGCAATCAAATTTCAGGTACCTGGCCCCATG
TTGAAAATGAGATCAAGATACACTCAGAGCTTACA
ACTTGAACTAATGATAAGAATCCTGTGTAAGCCCG
ATTCGCCACTTATGAAGGTCCATATCCCTGACAAG
GAAGGAAGAGGATGTCTTGTATCAGTATGGTTGCA
TGTATGCAATATCTTCAAATCAGGAAACAAGAATG
GCAGTGAGTGGCAGGAATACTGGATGAGAAAGTGT
GCTAACATGCAACTTGAAGTGTCGATTGCAGATAT
GTGGGGACCAACTATCATAATTCATGCCAGAGGTC
ACATTCCCAAAAGTGCTAAGTTGTTTTTTGGAAAG
GGTGGATGGAGCTGCCATCCACTTCACGAAGTTGT
TCCAAGTGTCACTAAAACACTATGGTCCGTGGGCT
GTGAGATTACAAAGGCGAAGGCAATAATACAAGAG
AGTAGCATCTCTCTTCTCGTGGAGACTACTGACAT
CATAAGTCCAAAAGTCAAAATTTCATCTAAGCATC
GCCGCTTTGGGAAATCAAATTGGGGTCTGTTCAAG
AAAACCAAATCACTGCCCAACCTGACGGAGCTGGA
ATGACTGACCCCCAATCGAGACTACACCACCTCAA
ACTATAGGTGGGTGGTACCTCAGTGATTAATCTCG
TAAGCACTGATCGTAGGCTACAACACACTAATATT
ATCCAGATTAGAGAGCTTAATTAGCTCTGTATTAA
TAATAACACTACTATTCCAATAACTGGAATCACCA
GCTTGATTTATCTCCAAAATGATTCAAAGAAAACA
AATCATATTAAGACTATCCTAAGCACGAACCCATA
TCGTCCTTCAAATCATGGGTACTATAATTCAATTT
CTGGTGGTCTCCTGTCTATTGGCAGGAGCAGGCAG
TCTTGATCCAGCAGCCCTCATGCAAATCGGTGTCA
TTCCAACAAATGTCCGGCAACTTATGTATTATACT
GAGGCCTCATCAGCATTCATTGTTGTGAAGTTAAT
GCCTACAATTGACTCGCCGATTAGTGGATGTAATA
TAACATCAATTTCAAGCTATAATGCAACAGTGACA
AAACTCCTACAGCCGATCGGTGAGAATTTGGAGAC
GATTAGGAACCAGTTGATTCCAACTCGGAGGAGAC
GCCGGTTTGCAGGGGTGGTGATTGGATTAGCTGCA
TTAGGAGTAGCTACTGCCGCACAGGTCACTGCCGC
AGTAGCACTAGTAAAGGCAAATGAAAATGCTGCGG
CTATACTCAATCTCAAAAATGCAATCCAAAAAACA
AATGCAGCAGTTGCAGATGTGGTCCAGGCCACACA
ATCACTAGGAACaGCAGTTCAAGCAGTTCAAGATC
ACATAAACAGTGTGGTAAGTCCAGCAATTACAGCA
GCCAATTGTAAAGCCCAAGATGCTATCATTGGCTC
AATCCTCAATCTCTATTTGACCGAGTTGACAACTA
TCTTCCACAATCAAATTACAAACCCTGCATTGAGT
CCTATTACAATTCAAGCTTTAAGGATCCTACTAGG
GAGTACCTTGCCGACTGTGGTCGAAAAATCTTTCA
ATACCCAGATAAGTGCGGCTGAGCTTCTCTCATCA
GGGTTATTGACAGGCCAGATTGTGGGATTAGATTT
GACCTATATGCAGATGGTCATAAAAATTGAGCTGC
CAACTTTAACTGTACAACCTGCAACCCAGATCATA
GATCTGGCCACCATTTCTGCATTCATTAACAATCA
AGAAGTCATGGCCCAATTACCAACACGTGTTATTG
TGACTGGCAGCTTGATCCAAGCCTATCCCGCATCG
CAATGCACTATTACACCCAACACTGTGTACTGTAG
GTATAATGATGCCCAAGTACTCTCAGATGATACGA
TGGCTTGCCTCCAAGGTAACTTGACAAGATGCACC
TTCTCTCCAGTGGTTGGGAGCTTTCTCACTCGATT
CGTGCTGTTCGATGGAATAGTTTATGCAAATTGCA
GGTCGATGTTGTGCAAGTGCATGCAGCCTGCTGCT
GTGATCCTACAGCCGAGTTCATCCCCTGTAACTGT
CATTGACATGTACAAATGTGTGAGTCTGCAGCTTG
ACAATCTCAGATTCACCATCACTCAATTGGCCAAT
GTAACCTACAATAGCACCATCAAGCTTGAAACATC
CCAGATCTTGCCTATTGATCCGTTGGATATATCCC
AGAATCTAGCTGCGGTGAATAAGAGTCTAAGTGAT
GCACTACAACACTTAGCACAAAGTGACACATACCT
TTCTGCAATCACATCAGCTACGACTACAAGTGTAT
TATCCATAATAGCAATCTGTCTTGGATCGTTAGGT
TTAATATTAATAATCTTGCTCAGTGTAGTTGTGTG
GAAGTTATTGACCATTGTCGCTGCTAATCGAAATA
GAATGGAGAATTTTGTTTATCATAATTCAGCATTC
CACCACTCACGATCTGATCTCAGTGAGAAAAATCA
ACCTGCAACTCTTGGAACAAGATAAGACAGTCATC
CATTAGTAATTTTTAAGAAAAAAACGATAGGACCG
AAACTAGTATTGAAAGAACCGTCTCGGTCAATCTA
GGTAATCGAGCTGcTACCGTCTCGGAAAGCTCAAA
TCATGCTGCCTGATCCGGAAGATCCGGAAAGCAAA
AAAGCTACAAGGAGAACAGGAAACCTAATTATCTG
CTTCCTATTCATCTTCTTTCCGTTTGTAAACTTCA
TTGTTCCAACTCTAAGACACTTGCTGTCCTAACAC
CTGCTATAGGCTATCCACTGCATCATCTCTTTTTA
AGAAAAAAATAGGCCCGGACGGGTTAGCAACAAGC
GACTGCCGGTGCCAACAACACAATCCACAATCTAC
AGTCGACGCGGCCGCCACCatgttcgtcttcctgg
tcctgctgcctctggtctcctcacagtgcgtcaat
ctgacaactcggactcagctgccacctgcttatac
taatagcttcaccagaggcgtgtactatcctgaca
aggtgtttagaagctccgtgctgcactctacacag
gatctgtttctgccattctttagcaacgtgacctg
gttccacgccatcagcggcaccaatggcacaaagc
ggttcgacaatcccgtgctgccttttaacgatggc
gtgtacttcgcctctaccgagaagagcaacatcat
cagaggctggatctttggcaccacactggactcca
agacacagtctctgctgatcgtgaacaatgccacc
aacgtggtcatcaaggtgtgcgagttccagttttg
taatgatcccttcctgggcgtgtatcacaagaaca
ataagagctggatggagtccgagtttagagtgtat
tctagcgccaacaactgcacatttgagtacgtgag
ccagcctttcctgatggacctggagggcaagcagg
gcaatttcaagaacctgagggagttcgtgtttaag
aatatcgacggctacttcaaaatctactctaagca
cacccccatcaacctggtgcgcgacctgcctcagg
gcttcagcgccctggagcccctggtggatctgcct
atcggcatcaacatcacccggtttcagacactgct
ggccctgcacagaagctacctgacacccggcgact
cctctagcggatggaccgccggcgctgccgcctac
tatgtgggctacctccagccccggaccttcctgct
gaagtacaacgagaatggcaccatcacagacgcag
tggattgcgccctggaTcccctgagcgagacaaag
tgtacactgaagtcctttaccgtggagaagggcat
ctatcagacatccaatttcagggtgcagccaaccg
agtctatcgtgcgctttcctaatatcacaaacctg
tgcccatttggcgaggtgttcaacgcaacccgctt
cgccagcgtgtacgcctggaataggaagcggatca
gcaactgcgtggccgactatagcgtgctgtacaac
tccgcctctttcagcacctttaagtgctatggcgt
gtcccccacaaagctgaatgacctgtgctttacca
acgtctacgccgattctttcgtgatcaggggcgac
gaggtgcgccagatcgcccccggccagacaggcaa
gatcgcagactacaattataagctgccagacgatt
tcaccggctgcgtgatcgcctggaacagcaacaat
ctggattccaaagtgggcggcaactacaattatct
gtaccggctgtttagaaagagcaatctgaagccct
tcgagagggacatctctacagaaatctaccaggcc
ggcagcaccccttgcaatggcgtggagggctttaa
ctgttatttcccactccagtcctacggcttccagc
ccacaTATggcgtgggctatcagccttaccgcgtg
gtggtgctgagctttgagctgctgcacgccccagc
aacagtgtgcggccccaagaagtccaccaatctgg
tgaagaacaagtgcgtgaacttcaacttcaacggc
ctgaccggcacaggcgtgctgaccgagtccaacaa
gaagttcctgccatttcagcagttcggcagggaca
tcgATgataccacagacgccgtgcgcgacccacag
accctggagatcctggacatcacaccctgctcttt
cggcggcgtgagcgtgatcacacccggcaccaata
caagcaaccaggtggccgtgctgtatcaggGcgtg
aattgtaccgaggtgcccgtggctatccacgccga
tcagctgaccccaacatggcgggtgtacagcaccg
gctccaacgtcttccagacaagagccggatgcctg
atcggagcagagcacgtgaacaattcctatgagtg
cgacatcccaatcggcgccggcatctgtgcctctt
accagacccagacaaactctcAcagaagagcccgg
agcgtggcctcccagtctatcatcgcctataccat
gtccctgggcgccgagaacagcgtggcctactcta
acaatagcatcgccatcccaaTcaacttcacaatc
tctgtgaccacagagatcctgcccgtgtccatgac
caagacatctgtggactgcacaatgtatatctgtg
gcgattctaccgagtgcagcaacctgctgctccag
tacggcagcttttgtacccagctgaatagagccct
gacaggcatcgccgtggagcaggataagaacacac
aggaggtgttcgcccaggtgaagcaaatctacaag
accccccctatcaaggactttggcggcttcaattt
ttcccagatcctgcctgatccatccaagccttcta
agcggagctttatcgaggacctgctgttcaacaag
gtgaccctggccgatgccggcttcatcaagcagta
tggcgattgcctgggcgacatcgcagccagggacc
tgatctgcgcccagaagtttaatggcctgaccgtg
ctgccacccctgctgacagatgagatgatcgcaca
gtacacaagcgccctgctggccggcaccatcacat
ccggatggaccttcggcgcaggagccgccctccag
atcccctttgccatgcagatggcctataggttcaa
cggcatcggcgtgacccagaatgtgctgtacgaga
accagaagctgatcgccaatcagtttaactccgcc
atcggcaagatccaggacagcctgtcctctacagc
cagcgccctgggcaagctccaggatgtggtgaatc
agaacgcccaggccctgaataccctggtgaagcag
ctgagcagcaacttcggcgccatctctagcgtgct
gaatgacatcctggcTcggctggacaaggtggagg
cagaggtgcagatcgaccggctgatcaccggccgg
ctccagagcctccagacctatgtgacacagcagct
gatcagggccgccgagatcagggccagcgccaatc
tggcagcaaccaagatgtccgagtgcgtgctgggc
cagtctaagagagtggacttttgtggcaagggcta
tcacctgatgtccttccctcagtctgccccacacg
gcgtggtgtttctgcacgtgacctacgtgcccgcc
caggagaagaacttcaccacagcccctgccatctg
ccacgatggcaaggcccactttccaagggagggcg
tgttcgtgtccaacggcacccactggtttgtgaca
cagcgcaatttctacgagccccagatcatcaccac
aCacaacaccttcgtgagcggcaactgtgacgtgg
tcatcggcatcgtgaacaataccgtgtatgatcca
ctccagcccgagctggacagctttaaggaggagct
ggataagtatttcaagaatcacacctcccctgacg
tggatctgggcgacatcagcggcatcaatgcctcc
gtggtgaacatccagaaggagatcgaccgcctgaa
cgaggtggctaagaatctgaacgagagcctgatcg
acctccaggagctgggcaagtatgagcagtacatc
aagtggccctggtacatctggctgggcttcatcgc
cggcctgatcgccatcgtgatggtgaccatcatgc
tgtgctgtatgacatcctgctgttcttgcctgaag
ggctgctgtagctgtggctcctgctgtAAGTTATT
GACCATTGTCGTTGCTAATCGAAATAGAATGGAGA
ATTTTGTTTATCATAAATGAGCTAGCCTCCTGCCA
TACTTCCTACTCACATCATATCTATTTTAAAGAAA
AAATAGGCCCGAACACTAATCGTGCCGGCAGTGCC
ACTGCACACACAACACTACACATACAATACACTAC
AATGGTTGCAGAAGATGCCCCTGTTAGGGGCACTT
GCCGAGTATTATTTCGAACAACAACTTTAATTTTT
CTATGCACACTATTAGCATTAAGCATCTCTATCCT
TTATGAGAGTTTAATAACCCAAAAGCAAATCATGA
GCCAAGCAGGCTCAACTGGATCTAATTCTAGATTA
GGAAGTATCACTGATCTTCTTAATAATATTCTCTC
TGTCGCAAATCAGATTATATATAACTCTGCAGTCG
CTCTACCTCTACAATTGGACACTCTTGAATCAACA
CTCCTTACAGCCATTAAGTCTCTTCAAACAAGTGA
CAAGCTAGAACAGAACTGCTCGTGGGGTGCTGCAC
TGATTAATGATAATAGATACATTAATGGCATCAAT
CAGTTCTATTTCTCAATTGCTGAGGGTCGCAAGCT
GACACTTGGCCCACTTCTTAATATACCTAGTTTCA
TTCCAACTGCCACGACACCAGAGGGCTGCACCAGG
ATCCCATCATTCTCGCTCACCAAGACACACTGGTG
TTATACACACAATGTTATCCTGAATGGATGCCAGG
ATCATGTATCCTCAAATCAATTTGTTTCCATGGGA
ATCATTGAACCCACTTCTGCCGGGTTTCCATCCTT
TCGAACCCTAAAGACTCTATATCTCAGCGATGGGG
TCAATCGTAAGAGCTGCTCTATCAGTACAGTTCCG
GGGGGTTGTATGATGTACTGTTTCGTCTCTACTCA
ACCAGAGAGGGATGACTACTTTTCTACCGCTCCTC
CAGAACAACGAATTATTATAATGTACTATAATGAT
ACAATCGTGGAGCGCATAATTAATCCACCCGGGGT
ACTAGATGTATGGGCAACATTGAACCCAGGAACAG
GAAGCGGGGTATATTATTTAGGTTGGGTACTCTTT
CCAATATATGGCGGCGTGATTAAAGATACGAGTTT
ATGGAATAATCAAGCAAATAAATACTTTATCCCCC
AGATGGTTGCTGCTCTCTGCTCACAAAACCAGGCA
ACTCAAGTCCAAAATGCTAAGTCATCATACTATAG
CAGCTGGTTTGGCAATCGAATGATTCAGTCTGGGA
TCCTGGCATGTCCTCTTCAACAGGATCTAACCAAT
GAGTGTTTAGTTCTGCCCTTTTCTAATGATCAGGT
GCTTATGGGTGCTGAAGGGAGATTATACATGTATG
GTGACTCGGTGTATTACTACCAAAGAAGCAATAGT
TGGTGGCCTATGACCATGCTGTATAAGGTAACCAT
AACATTCACTAATGGTCAGCCATCTGCTATATCAG
CTCAGAATGTGCCCACACAGCAGGTCCCTAGACCT
GGGACAGGAAGCTGCTCTGCAACAAATAGATGTCC
CGGTTTTTGCTTGAAAGGAGTGTATGCTGATGCCT
GGTTACTGACCAACCCTTCGTCTACCAGTACATTT
GGATCAGAAGCAACCTTCACTGGTTCTTATCTCAA
CGCAGCAACTCAGCGTATCAATCCGACGATGTATA
TCGCGAACAACACACAGATCATAAGCTCACAGCAA
TTTGGATCAAGCGGTCAAGAAGCAGCATATGGCCA
CACAACTTGTTTTAGGGACACAGGCTCTGTTATGG
TATACTGTATCTATATTATTGAATTGTCCTCATCT
CTCTTAGGACAATTTCAGATTGTCCCATTTATCCG
TCAGGTGACACTATCCTAAAGGCAGAAGCCTCCAG
GTCTGACCCAGCCAATCAAAGCATTATACCAGACC
ATGGAATGCATACCAAACATTATTGACACTAATGA
CACACAAAATTGGTTTTAAGAAAAACCAAGAGAAC
AATAGGCCAGAATGGCTGGGTCTCGGGAGATATTA
CTCCCTGAAGTCCATCTCAATTCACCAATTGTAAA
GCATAAGCTATACTATTACATTCTACTTGGAAACC
TCCCAAATGAGATCGACATTGACGATTTAGGTCCA
TTACATAATCAAAATTGGAATCAAATAGCACATGA
AGAGTCTAACTTAGCCCAACGCTTGGTAAATGTAA
GAAATTTTCTAATTACCCACATCCCTGATCTTAGA
AAGGGCCATTGGCAAGAGTATGTCAATGTAATACT
GTGGCCGCGAATTCTTCCCTTGATCCCGGATTTTA
AAATCAATGACCAATTGCCTCTACTCAAAAATTGG
GACAAGTTAGTTAAAGAATCATGTTCAGTAATCAA
TGCGGGTACTTCCCAGTGCATTCAGAATCTCAGCT
ATGGACTGACAGGTCGTGGGAACCTCTTTACACGA
TCACGTGAACTCTCTGGTGACCGCAGGGATATTGA
TCTTAAGACGGTTGTGGCAGCATGGCATGACTCAG
ACTGGAAAAGAATAAGTGATTTTTGGATTATGATC
AAATTCCAGATGAGACAATTAATTGTTAGGCAAAC
AGATCATAATGATCCTGATTTAATCACGTATATCG
AAAATAGAGAAGGCATAATCATCATAACCCCTGAA
CTGGTAGCATTATTTAACACTGAGAATCATACACT
AACATACATGACCTTTGAAATTGTACTGATGGTTT
CAGATATGTACGAAGGTCGTCACAACATTTTATCA
CTATGCACAGTTAGCACTTACCTGAATCCTCTGAA
GAAAAGAATAACATATTTATTGAGCCTTGTAGATA
ACTTAGCTTTTCAGATAGGTGATGCTGTATATAAC
ATAATTGCTTTGCTAGAATCCTTTGTATATGCACA
GTTGCAAATGTCAGATCCCATCCCAGAACTCAGAG
GACAATTCCATGCATTCGTATGTTCTGAGATTCTT
GATGCACTAAGGGGAACTAATAGTTTCACCCAGGA
TGAATCAAGAACTGTGACAACCAATTTGATATCCC
CATTCCAAGATCTGACCCCAGATCTTACGGCTGAA
TTGCTCTGTATAATGAGGCTTTGGGGACACCCCAT
GCTCACCGCCAGTCAAGCTGCGGGAAAGGTACGCG
AGTCCATGTGTGCTGGAAAAGTATTAGACTTTCCC
ACCATTATGAAAACACTAGCCTTTTTCCATACTAT
TCTGATCAATGGATACAGGAGGAAGCATCATGGAG
TATGGCCACCCTTAAACTTACCGGGTAATGCTTCA
AAGGGTCTCACGGAACTTATGAATGACAATACTGA
GATAAGCTATGAATTCACACTTAAGCATTGGAAGG
AAATCTCTCTTATAAAATTCAAGAAATGTTTTGAT
GCAGACGCAGGTGAGGAACTCAGTATATTTATGAA
AGATAAAGCAATTAGTGCCCCAAAACAAGATTGGA
TGAGTGTGTTTAGAAGAAGCCTAATCAAACAGCGC
CATCAGCATCATCAGGTCCCCCTACCAAATCCATT
CAATCGACGGCTATTGCTAAACTTTCTCGGAGATG
ACAAATTCGACCCGAATGTGGAGCTACAGTATGTA
ACATCAGGTGAGTATCTACATGATGACACGTTTTG
TGCATCATATTCACTAAAAGAGAAGGAAATTAAAC
CTGATGGTCGAATTTTTGCAAAGTTGACTAAGAGA
ATGAGATCATGTCAAGTTATAGCAGAATCTCTTTT
AGCGAACCATGCTGGGAAGTTAATGAAAGAGAATG
GTGTTGTGATGAATCAGCTATCATTAACAAAATCA
CTATTAACAATGAGTCAGATTGGAATAATATCCGA
GAAAGCTAGAAAATCGACTCGAGATAACATAAATC
AACCTGGTTTCCAGAATATCCAGAGAAATAAATCA
CATCACTCCAAGCAAGTCAATCAGCGAGATCCAAG
TGATGACTTTGAATTGGCAGCATCTTTTTTAACTA
CTGATCTCAAAAAATATTGTTTACAATGGAGGTAC
CAGACAATTATCCCATTTGCTCAATCATTAAACAG
AATGTATGGTTATCCTCATCTCTTTGAGTGGATTC
ACTTACGGCTAATGCGTAGTACACTTTACGTGGGG
GATCCCTTCAACCCACCAGCAGATACCAGTCAATT
TGATCTAGATAAAGTAATTAATGGAGATATCTTCA
TTGTATCACCCAGAGGTGGAATTGAAGGGCTATGT
CAAAAGGCTTGGACAATGATATCTATCTCTGTGAT
AATTCTATCTGCCACAGAGTCTGGCACACGAGTAA
TGAGTATGGTGCAGGGAGATAATCAAGCAATTGCT
GTCACCACACGAGTACCAAGGAGCCTGCCGACTCT
TGAGAAAAAGACTATTGCTTTTAGATCTTGTAATC
TATTCTTTGAGAGGTTAAAATGTAATAATTTTGGA
TTAGGTCACCATTTGAAAGAACAAGAGACTATCAT
TAGTTCTCACTTCTTTGTTTATAGCAAGAGAATAT
TCTATCAGGGGAGGATTCTAACGCAAGCCTTAAAA
AATGCTAGTAAGCTCTGCTTGACAGCTGATGTCCT
AGGAGAATGTACCCAATCATCATGTTCTAATCTTG
CAACTACTGTCATGAGGTTAACTGAGAATGGTGTT
GAAAAAGATATCTGTTTCTACTTGAATATCTATAT
GACCATCAAACAGCTCTCCTATGATATCATCTTCC
CTCAAGTGTCAATTCCTGGAGATCAGATCACATTA
GAATACATAAATAATCCACACCTGGTATCACGATT
GGCTCTTCTGCCATCCCAGCTAGGAGGTCTAAACT
ACCTGTCATGCAGTAGGCTGTTCAATCGAAACATA
GGCGACCCGGTGGTTTCCGCAGTTGCAGATCTTAA
GAGATTAATTAAATCAGGATGTATGGATTACTGGA
TCCTTTATAACTTATTAGGGAGAAAACCGGGAAAC
GGCTCATGGGCTACTTTAGCAGCTGACCCGTACTC
AATCAATATAGAGTATCAATACCCCCCAACTACAG
CTCTTAAGAGGCACACCCAACAAGCTCTGATGGAA
CTCAGTACGAATCCAATGTTACGTGGCATATTCTC
TGACAATGCACAGGCAGAAGAAAATAATCTTGCTA
GATTTCTCCTGGATAGGGAGGTGATCTTTCCGCGT
GTAGCTCACATCATCATTGAGCAAACCAGTGTCGG
GAGGAGAAAACAGATTCAAGGATATTTGGATTCAA
CTAGATCGATAATGAGTAAATCACTAGAAATTAAG
CCCTTGTCCAATAGGAAGCTTAATGAAATACTGGA
TTACAACATCAATTACCTAGCTTACAATTTGGCAT
TACTCAAGAATGCTATTGAACCTCCGACTTATTTG
AAAGCAATGACTCTTGAAACATGTAGCATCGACAT
TGCAAGGAGCCTCCGGAAGCTCTCCTGGGCCCCAC
TCTTGGGTGGGAGAAATCTTGAAGGATTAGAGACG
CCAGATCCCATTGAAATTACTGCAGGAGCATTAAT
TGTTGGATCGGGCTACTGTGAACAGTGTGCTGCAG
GAGACAATCGATTCACATGGTTTTTCTTGCCATCT
GGTATCGAGATAGGAGGGGATCCCCGTGATAATCC
TCCTATCCGTGTACCGTACATTGGCTCCAGGACTG
ATGAGAGGAGGGTAGCCTCAATGGCATACATCAGG
GGTGCCTCGAGTAGCCTAAAAGCAGTTCTTAGACT
GGCGGGAGTGTACATCTGGGCATTCGGAGATACTC
TGGAGAATTGGATAGATGCACTGGATTTGTCTCAC
ACTAGAGTTAACATCACACTTGAACAGCTGCAATC
CCTCACCCCACTTCCAACCTCTGCCAATCTAACCC
ATCGGTTGGATGATGGCACAACTACCCTAAAGTTT
ACTCCTGCGAGCTCTTACACCTTTTCAAGTTTCAC
TCATATATCAAATGATGAGCAATACCTGACAATTA
ATGACAAAACTGCAGATTCAAATATAATCTACCAA
CAGTTAATGATCACTGGACTCGGAATTTTAGAAAC
ATGGAATAATCCCCCAATCAATAGAACATTCGAAG
AATCTACCCTACATTTGCACACTGGTGCATCATGT
TGTGTCCGACCTGTGGACTCCTGCATCATCTCAGA
AGCATTAACAGTCAAGCCACATATTACAGTACCGT
ACAGCAATAAATTTGTATTTGATGAAGACCCGCTA
TCTGAATATGAGACTGCAAAACTGGAATCGTTATC
ATTTCAAGCCCAATTAGGCAACATTGATGCTGTAG
ATATGACAGGTAAATTAACATTATTGTCCCAATTC
ACTGCAAGGCAGATTATTAATGCAATCACTGGACT
CGATGAGTCTGTCTCTCTTACTAATGATGCCATTG
TTGCATCAGACTATGTCTCCAATTGGATTAGTGAA
TGCATGTATACCAAATTAGATGAATTATTTATGTA
TTGTGGGTGGGAACTACTATTGGAACTATCCTATC
AAATGTATTATCTGAGGGTAGTTGGGTGGAGTAAT
ATAGTGGATTATTCTTACATGATCTTGAGAAGAAT
CCCGGGTGCAGCATTAAACAATCTGGCATCTACAT
TAAGTCATCCAAAACTTTTCCGACGAGCTATCAAC
CTAGATATAGTTGCCCCCTTAAATGCTCCTCATTT
TGCATCTCTGGACTACATCAAGATGAGTGTGGATG
CAATACTCTGGGGCTGTAAAAGAGTCATCAATGTG
CTCTCCAATGGAGGGGACTTAGAATTAGTTGTGAC
ATCTGAAGATAGCCTTATTCTCAGTGACCGATCCA
TGAATCTCATTGCAAGGAAATTAACTTTATTATCA
CTGATTCACCATAATGGTTTGGAACTACCAAAGAT
TAAGGGGTTCTCTCCTGATGAGAAGTGTTTCGCTT
TGACAGAATTTTTGAGGAAAGTGGTGAACTCAGGG
TTGAGTTCAATAGAGAACCTATCAAATTTTATGTA
CAATGTGGAGAACCCACGGCTTGCAGCATTCGCCA
GCAACAATTACTACCTGACCAGAAAATTATTGAAT
TCAATACGAGATACTGAGTCGGGTCAAGTAGCAGT
CACCTCATATTATGAATCATTAGAATATATTGATA
GTCTTAAGCTAACCCCACATGTGCCTGGTACCTCA
TGCATTGAGGATGATAGTCTATGTACAAATGATTA
CATAATCTGGATCATAGAGTCTAATGCAAACTTGG
AGAAGTATCCAATTCCAAATAGCCCTGAGGATGAT
TCCAATTTCCATAACTTTAAGTTGAATGCTCCATC
GCACCATACCTTACGCCCATTAGGGTTGTCATCAA
CTGCTTGGTATAAGGGTATAAGCTGTTGCAGGTAC
CTTGAGCGATTAAAGCTACCACAAGGTGATCATTT
ATATATTGCAGAAGGTAGTGGTGCCAGTATGACAA
TCATAGAATACCTATTCCCAGGAAGAAAGATATAT
TACAATTCTTTATTTAGTAGTGGTGACAATCCCCC
ACAAAGAAATTATGCACCAATGCCTACTCAGTTCA
TTGAGAGTGTCCCATACAAGCTCTGGCAAGCACAC
ACAGATCAATATCCCGAGATTTTTGAGGACTTCAT
CCCTCTATGGAACGGAAATGCCGCCATGACTGACA
TAGGAATGACAGCTTGTGTAGAATTCATCATCAAT
CGAGTCGGCCCAAGGACTTGCAGTTTAGTACATGT
AGATTTGGAATCAAGTGCAAGCTTAAATCAACAAT
GCCTGTCAAAGCCGATAATTAATGCTATCATCACT
GCTACAACTGTTTTGTGCCCTCATGGGGTGCTTAT
TCTGAAATATAGTTGGTTGCCATTTACTAGATTTA
GTACTTTGATCACTTTCTTATGGTGCTACTTTGAG
AGAATCACTGTTCTTAGGAGCACATATTCTGGTCC
AGCTAATCATGAGGTTTATTTAATTTGTATCCTTG
CCAACAACTTTGCATTCCAGACTGTCTCGCAGGCA
ACAGGAATGGCGATGACTTTAACCGATCAAGGGTT
TACTTTGATATCACCTGAAAGAATAAATCAGTATT
GGGATGGTCACTTGAAGCAAGAACGTATCGTAGCA
GAAGCAATTGATAAGGTGGTTCTAGGAGAAGATGC
TCTATTCAATTCGAGTGATAATGAATTAATTCTCA
AATGTGGAGGGACACCAAATGCACGGAATCTTATC
GATATCGAGCCAGTCGCAACTTTCATAGAATTTGA
ACAACTGATCTGCACAATGTTGACAACCCACTTGA
AGGAAATAATTGATATAACAAGGTCTGGAACCCAG
GATTATGAAAGTTTATTACTCACTCCTTACAATTT
AGGTCTTCTTGGTAAAATCAGTACGATAGTGAGAT
TATTAACAGAAAGGATTCTAAATCATACTATCAGG
AATTGGTTGATCCTCCCACCTTCGCTCCGGATGAT
CGTGAAGCAGGACTTGGAATTCGGCATATTCAGGA
TTACTTCCATCCTCAATTCTGATCGGTTCCTGAAG
CTTTCTCCAAATAGGAAATACTTGATTACACAATT
AACTGCAGGCTACATTAGGAAATTGATTGAGGGGG
ATTGTAATATCGATCTAACCAGACCTATCCAAAAA
CAAATCTGGAAAGCATTAGGTTGTGTAGTCTATTG
TCACGATCCAGTAGATCAAAGGGAATCAACAGAGT
TTATTGATATAAATATTAATGAAGAAATAGACCGC
GGGATCGATGGCGAGGAAATCTAAATATATCAAGA
ATCAGAATTAGTTTAAGAAAAAAGAAGAGGATTAA
TCTTGGTTTTCCCCTTGGT.

4. CVL49

The nucleic acid sequence for CVL49 is:

(SEQ ID NO: 64)
ACCAGGGGGAAAACGAAGTGGTGACTCAAATCATC
GAAGACCCTCGAGATTACATAGGTCCGGAACCTAT
GGCCTTCGTGACCGACCTCGAGTCAGAGTAGTTCA
ATAAGGACCTATCAAGTTTGGGCAATTTTTCGTCC
CCGACACAAAAATGTCATCCGTGCTTAAAGCATAT
GAGCGATTCACACTCACTCAAGAACTGCAAGATCA
GAGTGAGGAAGGTACAATCCCACCTACAACACTAA
AACCGGTAATCAGGGTATTTATACTAACCTCTAAT
AACCCAGAGCTAAGATCCCGGCTTCTTCTATTCTG
CCTACGGATTGTTCTCAGTAATGGTGCAAGGGATT
CCCATCGCTTTGGAGCATTACTTACAATGTTTTCG
CTACCATCAGCCACAATGCTCAATCATGTCAAATT
AGCTGACCAGTCACCAGAAGCTGATATCGAAAGGG
TAGAGATCGATGGCTTTGAGGAGGGATCATTCCGC
TTAATCCCCAATGCTCGTTCAGGTATGAGCCGTGG
AGAGATCAATGCCTATGCTGCACTTGCAGAAGATC
TACCTGACACACTAAACCATGCAACACCTTTTGTT
GATTCCGAAGTCGAGGGAACTGCATGGGATGAGAT
TGAGACTTTCTTAGATATGTGTTACAGTGTCCTAA
TGCAGGCATGGATAGTGACTTGCAAGTGCATGACT
GCGCCAGACCAACCTGCTGCTTCTATTGAGAAACG
CCTGCAAAAATATCGTCAGCAAGGCAGGATCAACC
CGAGATATCTCCTGCAACCGGAGGCTCGAAGAATA
ATCCAGAATGTAATCCGAAAGGGAATGGTGGTCAG
ACATTTCCTCACCTTTGAACTGCAGCTTGCCCGAG
CACAAAGCCTTGTATCAAATAGGTATTATGCTATG
GTAGGGGATGTTGGAAAGTATATAGAGAATTGTGG
AATGGGAGGCTTCTTTTTGACACTAAAATATGCAT
TAGGAACCAGATGGCCCACACTTGCTTTAGCTGCA
TTTTCAGGAGAGCTAACAAAGCTAAAGTCCCTCAT
GGCATTATACCAGACCCTTGGTGAGCAGGCCCGAT
ATTTGGCCCTATTGGAGTCACCACATTTGATGGAT
TTTGCTGCAGCAAACTACCCACTGCTATATAGCTA
TGCTATGGGAATAGGCTATGTGTTAGATGTCAACA
TGAGGAACTACGCTTTCTCCAGATCATACATGAAT
AAGACATATTTCCAATTGGGAATGGAAACTGCAAG
AAAACAACAGGGTGCAGTTGACATGAGGATGGCAG
AAGATCTCGGTCTAACTCAAGCCGAACGCACCGAG
ATGGCAAATACACTTGCCAAATTGACCACAGCAAA
TCGAGGGGCAGACACCAGGGGAGGAGTCAACCCGT
TCTCATCTATCACTGGGACAACTCAGGTGCCCGCT
GCAGCAACAGGTGACACATTCGAGAGTTACATGGC
AGCGGATCGACTGAGGCAGAGATATGCTGATGCAG
GCACCCACGATGATGAGATGCCACCATTGGAAGAG
GAGGAAGAGGACGACACATCTGCAGGTCCACGCAC
TGGACTAACTCTTGAACAAGTGGCCTTGGACATCC
AGAACGCAGCAGTTGGAGCTCCCATCCATACAGAT
GACCTGAATGCCGCACTGGGTGATCTTGACATCTA
GACAATTCAGATCCCAATCTTAAATCGACACACCT
AATTGACCAGTTAGATGGAACTACAGTGGATTCCA
TAAGGTTCCTGCCTACCATCGGCTTTTAAGAAAAA
AATAGGCCCGGACGGGTTAGCAACAAGCGACTGCC
GATGCCAATAACACAATCCACAATCTACAATGGAT
CCCACTGATCTGAGCTTCTCCCCAGATGAGATCAA
TAAGCTCATAGAGACAGGCCTGAATACTGTGGAGT
ATTTTACTTCCCAACAAGTCACAGGAACATCCTCT
CTTGGAAAGAATACAATACCACCAGGGGTCACAGG
ACTACTAACCAATGCTGCAGAGGCAAAGATCCAAG
AGTCAACCAACCATCAGAAGGGTTCAGTTGGTGGG
GGCGCAAAACCAAAGAAACCGCGACCAAAAATTGC
CATTGTGCCAGCAGATGACAAAACGGTGCCCGGAA
AGCCGATCCCAAACCCTCTATTAGGTCTGGACTCC
ACCCCGAGCACCCAAACTGTGCTTGATCTAAGTGG
GAAAACATTACCATCAGGATCCTATAAGGGGGTTA
AACTTGCGAAATTTGGAAAAGAAAATCTGATGACA
CGGTTCATCGAGGAACCCAGAGAGAATCCTATCGC
AACCAATTCCCCCATCGATTTTAAGAGGGGCAGGG
ATACCGGCGGGTTCCATAGAAGGGAGTACTCAATC
GGATGGGTGGGAGATGAAGTCAAGGTCACTGAGTG
GTGCAATCCATCCTGTTCTCCAATCACCGCTGCAG
CAAGGCGATTTGAATGCACTTGTCACCAGTGTCCA
GTCACTTGCTCTGAATGTGAACGAGATACTTAATA
CAGTGAGAAATTTGGACTCTCGGATGAATCAACTG
GAGACAAAAGTAGATCGCATTCTCTCATCTCAGTC
TCTAATCCAGACCATCAAGAATGACATAGTTGGAC
TTAAAGCAGGGATGGCTACTTTAGAAGGAATGATT
ACAACTGTGAAAATCATGGACCCGGGAGTTCCCAG
TAATGTTACTGTGGAAGATGTACGCAAGAAACTAA
GTAACCATGCTGTTGTTGTGCCAGAATCATTCAAT
GATAGTTTCTTGACTCAATCTGAAGATGTAATTTC
ACTTGATGAGTTGGCTCGACCAACTGCAACAAGTG
TTAAGAAGATTGTCAGGAAGGTTCCTCCTCAGAAG
GATCTGACTGGATTGAAGATCACACTAGAGCAATT
GGCAAAGGATTGCATCAGCAAACCGAAGATGAGGG
AAGAGTATCTCCTCAAAATCAACCAGGCTTCCAGT
GAGGCTCAGCTAATTGACCTCAAGAAAGCAATCAT
CCGCAGTGCAATTTGATCAAGAAACACCCAATTAC
ACTACACTGGTATGACACTGTACTAACCCTGAGGG
TTTTAGAAAAAACGATTAACGATAAATAAGCCCGA
ACACTACACACCACCCGAGGCAGCCATGCCATCCA
TCAGCATCCCCGCAGACCCCACCAATCCACGTCAA
TCAATAAAAGCGTTCCCAATTGTGATCAACCGTGA
TGGGGGTGAGAAAGGTCGCTTGGTTAAACAACTAC
GCACAACCTACTTGAATGACCTAGATACTCATGAG
CCACTGGTGACATTCGTAAATACTTATGGATTCAT
CTACGAACAGGATCGGGGAAATACCATTGTCGGAG
AGGATCAACATGGGAAGAAAAGAGAGGCTGTGACT
GCTGCAATGGTTACCCTTGGATGTGGGCCTAATCT
ACCATCATTAGGGAATGTCCTGGGACAACTGAGTG
AATTCCAGGTCATTGTTAGGAAGACATCCAGCAAA
GCGGAAGAGATGGTCTTTGAAATTGTTAAGTATCC
GAGAATATTTCGGGGTCATACATTAATCCAGAAAG
GACTAGTCTGTGTCTCCGCAGAAAAATTTGTTAAG
TCACCAGGGAAAGTACAATCTGGAATGGACTATCT
CTTCATTCCGACATTTCTGTCAGTGACTTACTGTC
CAGCTGCAATCAAATTTCAGGTACCTGGCCCCATG
TTGAAAATGAGATCAAGATACACTCAGAGCTTACA
ACTTGAACTAATGATAAGAATCCTGTGTAAGCCCG
ATTCGCCACTTATGAAGGTCCATATCCCTGACAAG
GAAGGAAGAGGATGTCTTGTATCAGTATGGTTGCA
TGTATGCAATATCTTCAAATCAGGAAACAAGAATG
GCAGTGAGTGGCAGGAATACTGGATGAGAAAGTGT
GCTAACATGCAACTTGAAGTGTCGATTGCAGATAT
GTGGGGACCAACTATCATAATTCATGCCAGAGGTC
ACATTCCCAAAAGTGCTAAGTTGTTTTTTGGAAAG
GGTGGATGGAGCTGCCATCCACTTCACGAAGTTGT
TCCAAGTGTCACTAAAACACTATGGTCCGTGGGCT
GTGAGATTACAAAGGCGAAGGCAATAATACAAGAG
AGTAGCATCTCTCTTCTCGTGGAGACTACTGACAT
CATAAGTCCAAAAGTCAAAATTTCATCTAAGCATC
GCCGCTTTGGGAAATCAAATTGGGGTCTGTTCAAG
AAAACCAAATCACTGCCCAACCTGACGGAGCTGGA
ATGACTGACCCCCAATCGAGACTACACCACCTCAA
ACTATAGGTGGGTGGTACCTCAGTGATTAATCTCG
TAAGCACTGATCGTAGGCTACAACACACTAATATT
ATCCAGATTAGAGAGCTTAATTAGCTCTGTATTAA
TAATAACACTACTATTCCAATAACTGGAATCACCA
GCTTGATTTATCTCCAAAATGATTCAAAGAAAACA
AATCATATTAAGACTATCCTAAGCACGAACCCATA
TCGTCCTTCAAATCATGGGTACTATAATTCAATTT
CTGGTGGTCTCCTGTCTATTGGCAGGAGCAGGCAG
TCTTGATCCAGCAGCCCTCATGCAAATCGGTGTCA
TTCCAACAAATGTCCGGCAACTTATGTATTATACT
GAGGCCTCATCAGCATTCATTGTTGTGAAGTTAAT
GCCTACAATTGACTCGCCGATTAGTGGATGTAATA
TAACATCAATTTCAAGCTATAATGCAACAGTGACA
AAACTCCTACAGCCGATCGGTGAGAATTTGGAGAC
GATTAGGAACCAGTTGATTCCAACTCGGAGGAGAC
GCCGGTTTGCAGGGGTGGTGATTGGATTAGCTGCA
TTAGGAGTAGCTACTGCCGCACAGGTCACTGCCGC
AGTAGCACTAGTAAAGGCAAATGAAAATGCTGCGG
CTATACTCAATCTCAAAAATGCAATCCAAAAAACA
AATGCAGCAGTTGCAGATGTGGTCCAGGCCACACA
ATCACTAGGAACaGCAGTTCAAGCAGTTCAAGATC
ACATAAACAGTGTGGTAAGTCCAGCAATTACAGCA
GCCAATTGTAAAGCCCAAGATGCTATCATTGGCTC
AATCCTCAATCTCTATTTGACCGAGTTGACAACTA
TCTTCCACAATCAAATTACAAACCCTGCATTGAGT
CCTATTACAATTCAAGCTTTAAGGATCCTACTAGG
GAGTACCTTGCCGACTGTGGTCGAAAAATCTTTCA
ATACCCAGATAAGTGCGGCTGAGCTTCTCTCATCA
GGGTTATTGACAGGCCAGATTGTGGGATTAGATTT
GACCTATATGCAGATGGTCATAAAAATTGAGCTGC
CAACTTTAACTGTACAACCTGCAACCCAGATCATA
GATCTGGCCACCATTTCTGCATTCATTAACAATCA
AGAAGTCATGGCCCAATTACCAACACGTGTTATTG
TGACTGGCAGCTTGATCCAAGCCTATCCCGCATCG
CAATGCACTATTACACCCAACACTGTGTACTGTAG
GTATAATGATGCCCAAGTACTCTCAGATGATACGA
TGGCTTGCCTCCAAGGTAACTTGACAAGATGCACC
TTCTCTCCAGTGGTTGGGAGCTTTCTCACTCGATT
CGTGCTGTTCGATGGAATAGTTTATGCAAATTGCA
GGTCGATGTTGTGCAAGTGCATGCAGCCTGCTGCT
GTGATCCTACAGCCGAGTTCATCCCCTGTAACTGT
CATTGACATGTACAAATGTGTGAGTCTGCAGCTTG
ACAATCTCAGATTCACCATCACTCAATTGGCCAAT
GTAACCTACAATAGCACCATCAAGCTTGAAACATC
CCAGATCTTGCCTATTGATCCGTTGGATATATCCC
AGAATCTAGCTGCGGTGAATAAGAGTCTAAGTGAT
GCACTACAACACTTAGCACAAAGTGACACATACCT
TTCTGCAATCACATCAGCTACGACTACAAGTGTAT
TATCCATAATAGCAATCTGTCTTGGATCGTTAGGT
TTAATATTAATAATCTTGCTCAGTGTAGTTGTGTG
GAAGTTATTGACCATTGTCGCTGCTAATCGAAATA
GAATGGAGAATTTTGTTTATCATAATTCAGCATTC
CACCACTCACGATCTGATCTCAGTGAGAAAAATCA
ACCTGCAACTCTTGGAACAAGATAAGACAGTCATC
CATTAGTAATTTTTAAGAAAAAAACGATAGGACCG
AAACTAGTATTGAAAGAACCGTCTCGGTCAATCTA
GGTAATCGAGCTGcTACCGTCTCGGAAAGCTCAAA
TCATGCTGCCTGATCCGGAAGATCCGGAAAGCAAA
AAAGCTACAAGGAGAACAGGAAACCTAATTATCTG
CTTCCTATTCATCTTCTTTCCGTTTGTAAACTTCA
TTGTTCCAACTCTAAGACACTTGCTGTCCTAACAC
CTGCTATAGGCTATCCACTGCATCATCTCTTTTTA
AGAAAAAAATAGGCCCGGACGGGTTAGCAACAAGC
GACTGCCGGTGCCAACAACACAATCCACAATCTAC
AGTCGACGCGGCCGCCatgttcgtcttcctggtcc
tgctgcctctggtctcctcacagtgcgtcaatTTC
acaaAccggactcagctgccaTctgcttatactaa
tagcttcaccagaggcgtgtactatcctgacaagg
tgtttagaagctccgtgctgcactctacacaggat
ctgtttctgccattctttagcaacgtgacctggtt
ccacgccatccacgtgagcggcaccaatggcacaa
agcggttcgacaatcccgtgctgccttttaacgat
ggcgtgtacttcgcctctaccgagaagagcaacat
catcagaggctggatctttggcaccacactggact
ccaagacacagtctctgctgatcgtgaacaatgcc
accaacgtggtcatcaaggtgtgcgagttccagtt
ttgtaatTACcccttcctgggcgtgtactatcaca
agaacaataagagctggatggagtccgagtttaga
gtgtattctagcgccaacaactgcacatttgagta
cgtgagccagcctttcctgatggacctggagggca
agcagggcaatttcaagaacctgagCgagttcgtg
tttaagaatatcgacggctacttcaaaatctactc
taagcacacccccatcaacctggtgcgcgacctgc
ctcagggcttcagcgccctggagcccctggtggat
ctgcctatcggcatcaacatcacccggtttcagac
actgctggccctgcacagaagctacctgacacccg
gcgactcctctagcggatggaccgccggcgctgcc
gcctactatgtgggctacctccagccccggacctt
cctgctgaagtacaacgagaatggcaccatcacag
acgcagtggattgcgccctggaTcccctgagcgag
acaaagtgtacactgaagtcctttaccgtggagaa
gggcatctatcagacatccaatttcagggtgcagc
caaccgagtctatcgtgcgctttcctaatatcaca
aacctgtgcccatttggcgaggtgttcaacgcaac
ccgcttcgccagcgtgtacgcctggaataggaagc
ggatcagcaactgcgtggccgactatagcgtgctg
tacaactccgcctctttcagcacctttaagtgcta
tggcgtgtcccccacaaagctgaatgacctgtgct
ttaccaacgtctacgccgattctttcgtgatcagg
ggcgacgaggtgcgccagatcgcccccggccagac
aggcaCgatcgcagactacaattataagctgccag
acgatttcaccggctgcgtgatcgcctggaacagc
aacaatctggattccaaagtgggcggcaactacaa
ttatctgtaccggctgtttagaaagagcaatctga
agcccttcgagagggacatctctacagaaatctac
caggccggcagcaccccttgcaatggcgtgAaggg
ctttaactgttatttcccactccagtcctacggct
tccagcccacaTacggcgtgggctatcagccttac
cgcgtggtggtgctgagctttgagctgctgcacgc
cccagcaacagtgtgcggccccaagaagtccacca
atctggtgaagaacaagtgcgtgaacttcaacttc
aacggcctgaccggcacaggcgtgctgaccgagtc
caacaagaagttcctgccatttcagcagttcggca
gggacatcgcagataccacagacgccgtgcgcgac
ccacagaccctggagatcctggacatcacaccctg
ctctttcggcggcgtgagcgtgatcacacccggca
ccaatacaagcaaccaggtggccgtgctgtatcag
gGcgtgaattgtaccgaggtgcccgtggctatcca
cgccgatcagctgaccccaacatggcgggtgtaca
gcaccggctccaacgtcttccagacaagagccgga
tgcctgatcggagcagagTacgtgaacaattccta
tgagtgcgacatcccaatcggcgccggcatctgtg
cctcttaccagacccagacaaactctcccagaaga
gcccggagcgtggcctcccagtctatcatcgccta
taccatgtccctgggcgccgagaacagcgtggcct
actctaacaatagcatcgccatcccaaccaacttc
acaatctctgtgaccacagagatcctgcccgtgtc
catgaccaagacatctgtggactgcacaatgtata
tctgtggcgattctaccgagtgcagcaacctgctg
ctccagtacggcagcttttgtacccagctgaatag
agccctgacaggcatcgccgtggagcaggataaga
acacacaggaggtgttcgcccaggtgaagcaaatc
tacaagaccccccctatcaaggactttggcggctt
caatttttcccagatcctgcctgatccatccaagc
cttctaagcggagctttatcgaggacctgctgttc
aacaaggtgaccctggccgatgccggcttcatcaa
gcagtatggcgattgcctgggcgacatcgcagcca
gggacctgatctgcgcccagaagtttaatggcctg
accgtgctgccacccctgctgacagatgagatgat
cgcacagtacacaagcgccctgctggccggcacca
tcacatccggatggaccttcggcgcaggagccgcc
ctccagatcccctttgccatgcagatggcctatag
gttcaacggcatcggcgtgacccagaatgtgctgt
acgagaaccagaagctgatcgccaatcagtttaac
tccgccatcggcaagatccaggacagcctgtcctc
tacagccagcgccctgggcaagctccaggatgtgg
tgaatcagaacgcccaggccctgaataccctggtg
aagcagctgagcagcaacttcggcgccatctctag
cgtgctgaatgacatcctgagccggctggacaagg
tggaggcagaggtgcagatcgaccggctgatcacc
ggccggctccagagcctccagacctatgtgacaca
gcagctgatcagggccgccgagatcagggccagcg
ccaatctggcagcaaTTaagatgtccgagtgcgtg
ctgggccagtctaagagagtggacttttgtggcaa
gggctatcacctgatgtccttccctcagtctgccc
cacacggcgtggtgtttctgcacgtgacctacgtg
cccgcccaggagaagaacttcaccacagcccctgc
catctgccacgatggcaaggcccactttccaaggg
agggcgtgttcgtgtccaacggcacccactggttt
gtgacacagcgcaatttctacgagccccagatcat
caccacagacaacaccttcgtgagcggcaactgtg
acgtggtcatcggcatcgtgaacaataccgtgtat
gatccactccagcccgagctggacagctttaagga
ggagctggataagtatttcaagaatcacacctccc
ctgacgtggatctgggcgacatcagcggcatcaat
gcctccTTTgtgaacatccagaaggagatcgaccg
cctgaacgaggtggctaagaatctgaacgagagcc
tgatcgacctccaggagctgggcaagtatgagcag
tacatcaagtggccctggtacatctggctgggctt
catcgccggcctgatcgccatcgtgatggtgacca
tcatgctgtgctgtatgacatcctgctgttcttgc
ctgaagggctgctgtagctgtggctcctgctgtAA
GTTATTGACCATTGTCGTTGCTAATCGAAATAGAA
TGGAGAATTTTGTTTATCATAAATGAGCTAGCCTC
CTGCCATACTTCCTACTCACATCATATCTATTTTA
AAGAAAAAATAGGCCCGAACACTAATCGTGCCGGC
AGTGCCACTGCACACACAACACTACACATACAATA
CACTACAATGGTTGCAGAAGATGCCCCTGTTAGGG
GCACTTGCCGAGTATTATTTCGAACAACAACTTTA
ATTTTTCTATGCACACTATTAGCATTAAGCATCTC
TATCCTTTATGAGAGTTTAATAACCCAAAAGCAAA
TCATGAGCCAAGCAGGCTCAACTGGATCTAATTCT
AGATTAGGAAGTATCACTGATCTTCTTAATAATAT
TCTCTCTGTCGCAAATCAGATTATATATAACTCTG
CAGTCGCTCTACCTCTACAATTGGACACTCTTGAA
TCAACACTCCTTACAGCCATTAAGTCTCTTCAAAC
AAGTGACAAGCTAGAACAGAACTGCTCGTGGGGTG
CTGCACTGATTAATGATAATAGATACATTAATGGC
ATCAATCAGTTCTATTTCTCAATTGCTGAGGGTCG
CAAGCTGACACTTGGCCCACTTCTTAATATACCTA
GTTTCATTCCAACTGCCACGACACCAGAGGGCTGC
ACCAGGATCCCATCATTCTCGCTCACCAAGACACA
CTGGTGTTATACACACAATGTTATCCTGAATGGAT
GCCAGGATCATGTATCCTCAAATCAATTTGTTTCC
ATGGGAATCATTGAACCCACTTCTGCCGGGTTTCC
ATCCTTTCGAACCCTAAAGACTCTATATCTCAGCG
ATGGGGTCAATCGTAAGAGCTGCTCTATCAGTACA
GTTCCGGGGGGTTGTATGATGTACTGTTTCGTCTC
TACTCAACCAGAGAGGGATGACTACTTTTCTACCG
CTCCTCCAGAACAACGAATTATTATAATGTACTAT
AATGATACAATCGTGGAGCGCATAATTAATCCACC
CGGGGTACTAGATGTATGGGCAACATTGAACCCAG
GAACAGGAAGCGGGGTATATTATTTAGGTTGGGTA
CTCTTTCCAATATATGGCGGCGTGATTAAAGATAC
GAGTTTATGGAATAATCAAGCAAATAAATACTTTA
TCCCCCAGATGGTTGCTGCTCTCTGCTCACAAAAC
CAGGCAACTCAAGTCCAAAATGCTAAGTCATCATA
CTATAGCAGCTGGTTTGGCAATCGAATGATTCAGT
CTGGGATCCTGGCATGTCCTCTTCAACAGGATCTA
ACCAATGAGTGTTTAGTTCTGCCCTTTTCTAATGA
TCAGGTGCTTATGGGTGCTGAAGGGAGATTATACA
TGTATGGTGACTCGGTGTATTACTACCAAAGAAGC
AATAGTTGGTGGCCTATGACCATGCTGTATAAGGT
AACCATAACATTCACTAATGGTCAGCCATCTGCTA
TATCAGCTCAGAATGTGCCCACACAGCAGGTCCCT
AGACCTGGGACAGGAAGCTGCTCTGCAACAAATAG
ATGTCCCGGTTTTTGCTTGAAAGGAGTGTATGCTG
ATGCCTGGTTACTGACCAACCCTTCGTCTACCAGT
ACATTTGGATCAGAAGCAACCTTCACTGGTTCTTA
TCTCAACGCAGCAACTCAGCGTATCAATCCGACGA
TGTATATCGCGAACAACACACAGATCATAAGCTCA
CAGCAATTTGGATCAAGCGGTCAAGAAGCAGCATA
TGGCCACACAACTTGTTTTAGGGACACAGGCTCTG
TTATGGTATACTGTATCTATATTATTGAATTGTCC
TCATCTCTCTTAGGACAATTTCAGATTGTCCCATT
TATCCGTCAGGTGACACTATCCTAAAGGCAGAAGC
CTCCAGGTCTGACCCAGCCAATCAAAGCATTATAC
CAGACCATGGAATGCATACCAAACATTATTGACAC
TAATGACACACAAAATTGGTTTTAAGAAAAACCAA
GAGAACAATAGGCCAGAATGGCTGGGTCTCGGGAG
ATATTACTCCCTGAAGTCCATCTCAATTCACCAAT
TGTAAAGCATAAGCTATACTATTACATTCTACTTG
GAAACCTCCCAAATGAGATCGACATTGACGATTTA
GGTCCATTACATAATCAAAATTGGAATCAAATAGC
ACATGAAGAGTCTAACTTAGCCCAACGCTTGGTAA
ATGTAAGAAATTTTCTAATTACCCACATCCCTGAT
CTTAGAAAGGGCCATTGGCAAGAGTATGTCAATGT
AATACTGTGGCCGCGAATTCTTCCCTTGATCCCGG
ATTTTAAAATCAATGACCAATTGCCTCTACTCAAA
AATTGGGACAAGTTAGTTAAAGAATCATGTTCAGT
AATCAATGCGGGTACTTCCCAGTGCATTCAGAATC
TCAGCTATGGACTGACAGGTCGTGGGAACCTCTTT
ACACGATCACGTGAACTCTCTGGTGACCGCAGGGA
TATTGATCTTAAGACGGTTGTGGCAGCATGGCATG
ACTCAGACTGGAAAAGAATAAGTGATTTTTGGATT
ATGATCAAATTCCAGATGAGACAATTAATTGTTAG
GCAAACAGATCATAATGATCCTGATTTAATCACGT
ATATCGAAAATAGAGAAGGCATAATCATCATAACC
CCTGAACTGGTAGCATTATTTAACACTGAGAATCA
TACACTAACATACATGACCTTTGAAATTGTACTGA
TGGTTTCAGATATGTACGAAGGTCGTCACAACATT
TTATCACTATGCACAGTTAGCACTTACCTGAATCC
TCTGAAGAAAAGAATAACATATTTATTGAGCCTTG
TAGATAACTTAGCTTTTCAGATAGGTGATGCTGTA
TATAACATAATTGCTTTGCTAGAATCCTTTGTATA
TGCACAGTTGCAAATGTCAGATCCCATCCCAGAAC
TCAGAGGACAATTCCATGCATTCGTATGTTCTGAG
ATTCTTGATGCACTAAGGGGAACTAATAGTTTCAC
CCAGGATGAATCAAGAACTGTGACAACCAATTTGA
TATCCCCATTCCAAGATCTGACCCCAGATCTTACG
GCTGAATTGCTCTGTATAATGAGGCTTTGGGGACA
CCCCATGCTCACCGCCAGTCAAGCTGCGGGAAAGG
TACGCGAGTCCATGTGTGCTGGAAAAGTATTAGAC
TTTCCCACCATTATGAAAACACTAGCCTTTTTCCA
TACTATTCTGATCAATGGATACAGGAGGAAGCATC
ATGGAGTATGGCCACCCTTAAACTTACCGGGTAAT
GCTTCAAAGGGTCTCACGGAACTTATGAATGACAA
TACTGAGATAAGCTATGAATTCACACTTAAGCATT
GGAAGGAAATCTCTCTTATAAAATTCAAGAAATGT
TTTGATGCAGACGCAGGTGAGGAACTCAGTATATT
TATGAAAGATAAAGCAATTAGTGCCCCAAAACAAG
ATTGGATGAGTGTGTTTAGAAGAAGCCTAATCAAA
CAGCGCCATCAGCATCATCAGGTCCCCCTACCAAA
TCCATTCAATCGACGGCTATTGCTAAACTTTCTCG
GAGATGACAAATTCGACCCGAATGTGGAGCTACAG
TATGTAACATCAGGTGAGTATCTACATGATGACAC
GTTTTGTGCATCATATTCACTAAAAGAGAAGGAAA
TTAAACCTGATGGTCGAATTTTTGCAAAGTTGACT
AAGAGAATGAGATCATGTCAAGTTATAGCAGAATC
TCTTTTAGCGAACCATGCTGGGAAGTTAATGAAAG
AGAATGGTGTTGTGATGAATCAGCTATCATTAACA
AAATCACTATTAACAATGAGTCAGATTGGAATAAT
ATCCGAGAAAGCTAGAAAATCGACTCGAGATAACA
TAAATCAACCTGGTTTCCAGAATATCCAGAGAAAT
AAATCACATCACTCCAAGCAAGTCAATCAGCGAGA
TCCAAGTGATGACTTTGAATTGGCAGCATCTTTTT
TAACTACTGATCTCAAAAAATATTGTTTACAATGG
AGGTACCAGACAATTATCCCATTTGCTCAATCATT
AAACAGAATGTATGGTTATCCTCATCTCTTTGAGT
GGATTCACTTACGGCTAATGCGTAGTACACTTTAC
GTGGGGGATCCCTTCAACCCACCAGCAGATACCAG
TCAATTTGATCTAGATAAAGTAATTAATGGAGATA
TCTTCATTGTATCACCCAGAGGTGGAATTGAAGGG
CTATGTCAAAAGGCTTGGACAATGATATCTATCTC
TGTGATAATTCTATCTGCCACAGAGTCTGGCACAC
GAGTAATGAGTATGGTGCAGGGAGATAATCAAGCA
ATTGCTGTCACCACACGAGTACCAAGGAGCCTGCC
GACTCTTGAGAAAAAGACTATTGCTTTTAGATCTT
GTAATCTATTCTTTGAGAGGTTAAAATGTAATAAT
TTTGGATTAGGTCACCATTTGAAAGAACAAGAGAC
TATCATTAGTTCTCACTTCTTTGTTTATAGCAAGA
GAATATTCTATCAGGGGAGGATTCTAACGCAAGCC
TTAAAAAATGCTAGTAAGCTCTGCTTGACAGCTGA
TGTCCTAGGAGAATGTACCCAATCATCATGTTCTA
ATCTTGCAACTACTGTCATGAGGTTAACTGAGAAT
GGTGTTGAAAAAGATATCTGTTTCTACTTGAATAT
CTATATGACCATCAAACAGCTCTCCTATGATATCA
TCTTCCCTCAAGTGTCAATTCCTGGAGATCAGATC
ACATTAGAATACATAAATAATCCACACCTGGTATC
ACGATTGGCTCTTCTGCCATCCCAGCTAGGAGGTC
TAAACTACCTGTCATGCAGTAGGCTGTTCAATCGA
AACATAGGCGACCCGGTGGTTTCCGCAGTTGCAGA
TCTTAAGAGATTAATTAAATCAGGATGTATGGATT
ACTGGATCCTTTATAACTTATTAGGGAGAAAACCG
GGAAACGGCTCATGGGCTACTTTAGCAGCTGACCC
GTACTCAATCAATATAGAGTATCAATACCCCCCAA
CTACAGCTCTTAAGAGGCACACCCAACAAGCTCTG
ATGGAACTCAGTACGAATCCAATGTTACGTGGCAT
ATTCTCTGACAATGCACAGGCAGAAGAAAATAATC
TTGCTAGATTTCTCCTGGATAGGGAGGTGATCTTT
CCGCGTGTAGCTCACATCATCATTGAGCAAACCAG
TGTCGGGAGGAGAAAACAGATTCAAGGATATTTGG
ATTCAACTAGATCGATAATGAGTAAATCACTAGAA
ATTAAGCCCTTGTCCAATAGGAAGCTTAATGAAAT
ACTGGATTACAACATCAATTACCTAGCTTACAATT
TGGCATTACTCAAGAATGCTATTGAACCTCCGACT
TATTTGAAAGCAATGACTCTTGAAACATGTAGCAT
CGACATTGCAAGGAGCCTCCGGAAGCTCTCCTGGG
CCCCACTCTTGGGTGGGAGAAATCTTGAAGGATTA
GAGACGCCAGATCCCATTGAAATTACTGCAGGAGC
ATTAATTGTTGGATCGGGCTACTGTGAACAGTGTG
CTGCAGGAGACAATCGATTCACATGGTTTTTCTTG
CCATCTGGTATCGAGATAGGAGGGGATCCCCGTGA
TAATCCTCCTATCCGTGTACCGTACATTGGCTCCA
GGACTGATGAGAGGAGGGTAGCCTCAATGGCATAC
ATCAGGGGTGCCTCGAGTAGCCTAAAAGCAGTTCT
TAGACTGGCGGGAGTGTACATCTGGGCATTCGGAG
ATACTCTGGAGAATTGGATAGATGCACTGGATTTG
TCTCACACTAGAGTTAACATCACACTTGAACAGCT
GCAATCCCTCACCCCACTTCCAACCTCTGCCAATC
TAACCCATCGGTTGGATGATGGCACAACTACCCTA
AAGTTTACTCCTGCGAGCTCTTACACCTTTTCAAG
TTTCACTCATATATCAAATGATGAGCAATACCTGA
CAATTAATGACAAAACTGCAGATTCAAATATAATC
TACCAACAGTTAATGATCACTGGACTCGGAATTTT
AGAAACATGGAATAATCCCCCAATCAATAGAACAT
TCGAAGAATCTACCCTACATTTGCACACTGGTGCA
TCATGTTGTGTCCGACCTGTGGACTCCTGCATCAT
CTCAGAAGCATTAACAGTCAAGCCACATATTACAG
TACCGTACAGCAATAAATTTGTATTTGATGAAGAC
CCGCTATCTGAATATGAGACTGCAAAACTGGAATC
GTTATCATTTCAAGCCCAATTAGGCAACATTGATG
CTGTAGATATGACAGGTAAATTAACATTATTGTCC
CAATTCACTGCAAGGCAGATTATTAATGCAATCAC
TGGACTCGATGAGTCTGTCTCTCTTACTAATGATG
CCATTGTTGCATCAGACTATGTCTCCAATTGGATT
AGTGAATGCATGTATACCAAATTAGATGAATTATT
TATGTATTGTGGGTGGGAACTACTATTGGAACTAT
CCTATCAAATGTATTATCTGAGGGTAGTTGGGTGG
AGTAATATAGTGGATTATTCTTACATGATCTTGAG
AAGAATCCCGGGTGCAGCATTAAACAATCTGGCAT
CTACATTAAGTCATCCAAAACTTTTCCGACGAGCT
ATCAACCTAGATATAGTTGCCCCCTTAAATGCTCC
TCATTTTGCATCTCTGGACTACATCAAGATGAGTG
TGGATGCAATACTCTGGGGCTGTAAAAGAGTCATC
AATGTGCTCTCCAATGGAGGGGACTTAGAATTAGT
TGTGACATCTGAAGATAGCCTTATTCTCAGTGACC
GATCCATGAATCTCATTGCAAGGAAATTAACTTTA
TTATCACTGATTCACCATAATGGTTTGGAACTACC
AAAGATTAAGGGGTTCTCTCCTGATGAGAAGTGTT
TCGCTTTGACAGAATTTTTGAGGAAAGTGGTGAAC
TCAGGGTTGAGTTCAATAGAGAACCTATCAAATTT
TATGTACAATGTGGAGAACCCACGGCTTGCAGCAT
TCGCCAGCAACAATTACTACCTGACCAGAAAATTA
TTGAATTCAATACGAGATACTGAGTCGGGTCAAGT
AGCAGTCACCTCATATTATGAATCATTAGAATATA
TTGATAGTCTTAAGCTAACCCCACATGTGCCTGGT
ACCTCATGCATTGAGGATGATAGTCTATGTACAAA
TGATTACATAATCTGGATCATAGAGTCTAATGCAA
ACTTGGAGAAGTATCCAATTCCAAATAGCCCTGAG
GATGATTCCAATTTCCATAACTTTAAGTTGAATGC
TCCATCGCACCATACCTTACGCCCATTAGGGTTGT
CATCAACTGCTTGGTATAAGGGTATAAGCTGTTGC
AGGTACCTTGAGCGATTAAAGCTACCACAAGGTGA
TCATTTATATATTGCAGAAGGTAGTGGTGCCAGTA
TGACAATCATAGAATACCTATTCCCAGGAAGAAAG
ATATATTACAATTCTTTATTTAGTAGTGGTGACAA
TCCCCCACAAAGAAATTATGCACCAATGCCTACTC
AGTTCATTGAGAGTGTCCCATACAAGCTCTGGCAA
GCACACACAGATCAATATCCCGAGATTTTTGAGGA
CTTCATCCCTCTATGGAACGGAAATGCCGCCATGA
CTGACATAGGAATGACAGCTTGTGTAGAATTCATC
ATCAATCGAGTCGGCCCAAGGACTTGCAGTTTAGT
ACATGTAGATTTGGAATCAAGTGCAAGCTTAAATC
AACAATGCCTGTCAAAGCCGATAATTAATGCTATC
ATCACTGCTACAACTGTTTTGTGCCCTCATGGGGT
GCTTATTCTGAAATATAGTTGGTTGCCATTTACTA
GATTTAGTACTTTGATCACTTTCTTATGGTGCTAC
TTTGAGAGAATCACTGTTCTTAGGAGCACATATTC
TGGTCCAGCTAATCATGAGGTTTATTTAATTTGTA
TCCTTGCCAACAACTTTGCATTCCAGACTGTCTCG
CAGGCAACAGGAATGGCGATGACTTTAACCGATCA
AGGGTTTACTTTGATATCACCTGAAAGAATAAATC
AGTATTGGGATGGTCACTTGAAGCAAGAACGTATC
GTAGCAGAAGCAATTGATAAGGTGGTTCTAGGAGA
AGATGCTCTATTCAATTCGAGTGATAATGAATTAA
TTCTCAAATGTGGAGGGACACCAAATGCACGGAAT
CTTATCGATATCGAGCCAGTCGCAACTTTCATAGA
ATTTGAACAACTGATCTGCACAATGTTGACAACCC
ACTTGAAGGAAATAATTGATATAACAAGGTCTGGA
ACCCAGGATTATGAAAGTTTATTACTCACTCCTTA
CAATTTAGGTCTTCTTGGTAAAATCAGTACGATAG
TGAGATTATTAACAGAAAGGATTCTAAATCATACT
ATCAGGAATTGGTTGATCCTCCCACCTTCGCTCCG
GATGATCGTGAAGCAGGACTTGGAATTCGGCATAT
TCAGGATTACTTCCATCCTCAATTCTGATCGGTTC
CTGAAGCTTTCTCCAAATAGGAAATACTTGATTAC
ACAATTAACTGCAGGCTACATTAGGAAATTGATTG
AGGGGGATTGTAATATCGATCTAACCAGACCTATC
CAAAAACAAATCTGGAAAGCATTAGGTTGTGTAGT
CTATTGTCACGATCCAGTAGATCAAAGGGAATCAA
CAGAGTTTATTGATATAAATATTAATGAAGAAATA
GACCGCGGGATCGATGGCGAGGAAATCTAAATATA
TCAAGAATCAGAATTAGTTTAAGAAAAAAGAAGAG
GATTAATCTTGGTTTTCCCCTTGGT.

5. CVL50

The nucleic acid sequence for CVL50 is:

(SEQ ID NO: 65)
ACCAGGGGGAAAACGAAGTGGTGACTCAAATCATC
GAAGACCCTCGAGATTACATAGGTCCGGAACCTAT
GGCCTTCGTGACCGACCTCGAGTCAGAGTAGTTCA
ATAAGGACCTATCAAGTTTGGGCAATTTTTCGTCC
CCGACACAAAAATGTCATCCGTGCTTAAAGCATAT
GAGCGATTCACACTCACTCAAGAACTGCAAGATCA
GAGTGAGGAAGGTACAATCCCACCTACAACACTAA
AACCGGTAATCAGGGTATTTATACTAACCTCTAAT
AACCCAGAGCTAAGATCCCGGCTTCTTCTATTCTG
CCTACGGATTGTTCTCAGTAATGGTGCAAGGGATT
CCCATCGCTTTGGAGCATTACTTACAATGTTTTCG
CTACCATCAGCCACAATGCTCAATCATGTCAAATT
AGCTGACCAGTCACCAGAAGCTGATATCGAAAGGG
TAGAGATCGATGGCTTTGAGGAGGGATCATTCCGC
TTAATCCCCAATGCTCGTTCAGGTATGAGCCGTGG
AGAGATCAATGCCTATGCTGCACTTGCAGAAGATC
TACCTGACACACTAAACCATGCAACACCTTTTGTT
GATTCCGAAGTCGAGGGAACTGCATGGGATGAGAT
TGAGACTTTCTTAGATATGTGTTACAGTGTCCTAA
TGCAGGCATGGATAGTGACTTGCAAGTGCATGACT
GCGCCAGACCAACCTGCTGCTTCTATTGAGAAACG
CCTGCAAAAATATCGTCAGCAAGGCAGGATCAACC
CGAGATATCTCCTGCAACCGGAGGCTCGAAGAATA
ATCCAGAATGTAATCCGAAAGGGAATGGTGGTCAG
ACATTTCCTCACCTTTGAACTGCAGCTTGCCCGAG
CACAAAGCCTTGTATCAAATAGGTATTATGCTATG
GTAGGGGATGTTGGAAAGTATATAGAGAATTGTGG
AATGGGAGGCTTCTTTTTGACACTAAAATATGCAT
TAGGAACCAGATGGCCCACACTTGCTTTAGCTGCA
TTTTCAGGAGAGCTAACAAAGCTAAAGTCCCTCAT
GGCATTATACCAGACCCTTGGTGAGCAGGCCCGAT
ATTTGGCCCTATTGGAGTCACCACATTTGATGGAT
TTTGCTGCAGCAAACTACCCACTGCTATATAGCTA
TGCTATGGGAATAGGCTATGTGTTAGATGTCAACA
TGAGGAACTACGCTTTCTCCAGATCATACATGAAT
AAGACATATTTCCAATTGGGAATGGAAACTGCAAG
AAAACAACAGGGTGCAGTTGACATGAGGATGGCAG
AAGATCTCGGTCTAACTCAAGCCGAACGCACCGAG
ATGGCAAATACACTTGCCAAATTGACCACAGCAAA
TCGAGGGGCAGACACCAGGGGAGGAGTCAACCCGT
TCTCATCTATCACTGGGACAACTCAGGTGCCCGCT
GCAGCAACAGGTGACACATTCGAGAGTTACATGGC
AGCGGATCGACTGAGGCAGAGATATGCTGATGCAG
GCACCCACGATGATGAGATGCCACCATTGGAAGAG
GAGGAAGAGGACGACACATCTGCAGGTCCACGCAC
TGGACTAACTCTTGAACAAGTGGCCTTGGACATCC
AGAACGCAGCAGTTGGAGCTCCCATCCATACAGAT
GACCTGAATGCCGCACTGGGTGATCTTGACATCTA
GACAATTCAGATCCCAATCTTAAATCGACACACCT
AATTGACCAGTTAGATGGAACTACAGTGGATTCCA
TAAGGTTCCTGCCTACCATCGGCTTTTAAGAAAAA
AATAGGCCCGGACGGGTTAGCAACAAGCGACTGCC
GATGCCAATAACACAATCCACAATCTACAATGGAT
CCCACTGATCTGAGCTTCTCCCCAGATGAGATCAA
TAAGCTCATAGAGACAGGCCTGAATACTGTGGAGT
ATTTTACTTCCCAACAAGTCACAGGAACATCCTCT
CTTGGAAAGAATACAATACCACCAGGGGTCACAGG
ACTACTAACCAATGCTGCAGAGGCAAAGATCCAAG
AGTCAACCAACCATCAGAAGGGTTCAGTTGGTGGG
GGCGCAAAACCAAAGAAACCGCGACCAAAAATTGC
CATTGTGCCAGCAGATGACAAAACGGTGCCCGGAA
AGCCGATCCCAAACCCTCTATTAGGTCTGGACTCC
ACCCCGAGCACCCAAACTGTGCTTGATCTAAGTGG
GAAAACATTACCATCAGGATCCTATAAGGGGGTTA
AACTTGCGAAATTTGGAAAAGAAAATCTGATGACA
CGGTTCATCGAGGAACCCAGAGAGAATCCTATCGC
AACCAATTCCCCCATCGATTTTAAGAGGGGCAGGG
ATACCGGCGGGTTCCATAGAAGGGAGTACTCAATC
GGATGGGTGGGAGATGAAGTCAAGGTCACTGAGTG
GTGCAATCCATCCTGTTCTCCAATCACCGCTGCAG
CAAGGCGATTTGAATGCACTTGTCACCAGTGTCCA
GTCACTTGCTCTGAATGTGAACGAGATACTTAATA
CAGTGAGAAATTTGGACTCTCGGATGAATCAACTG
GAGACAAAAGTAGATCGCATTCTCTCATCTCAGTC
TCTAATCCAGACCATCAAGAATGACATAGTTGGAC
TTAAAGCAGGGATGGCTACTTTAGAAGGAATGATT
ACAACTGTGAAAATCATGGACCCGGGAGTTCCCAG
TAATGTTACTGTGGAAGATGTACGCAAGAAACTAA
GTAACCATGCTGTTGTTGTGCCAGAATCATTCAAT
GATAGTTTCTTGACTCAATCTGAAGATGTAATTTC
ACTTGATGAGTTGGCTCGACCAACTGCAACAAGTG
TTAAGAAGATTGTCAGGAAGGTTCCTCCTCAGAAG
GATCTGACTGGATTGAAGATCACACTAGAGCAATT
GGCAAAGGATTGCATCAGCAAACCGAAGATGAGGG
AAGAGTATCTCCTCAAAATCAACCAGGCTTCCAGT
GAGGCTCAGCTAATTGACCTCAAGAAAGCAATCAT
CCGCAGTGCAATTTGATCAAGAAACACCCAATTAC
ACTACACTGGTATGACACTGTACTAACCCTGAGGG
TTTTAGAAAAAACGATTAACGATAAATAAGCCCGA
ACACTACACACCACCCGAGGCAGCCATGCCATCCA
TCAGCATCCCCGCAGACCCCACCAATCCACGTCAA
TCAATAAAAGCGTTCCCAATTGTGATCAACCGTGA
TGGGGGTGAGAAAGGTCGCTTGGTTAAACAACTAC
GCACAACCTACTTGAATGACCTAGATACTCATGAG
CCACTGGTGACATTCGTAAATACTTATGGATTCAT
CTACGAACAGGATCGGGGAAATACCATTGTCGGAG
AGGATCAACATGGGAAGAAAAGAGAGGCTGTGACT
GCTGCAATGGTTACCCTTGGATGTGGGCCTAATCT
ACCATCATTAGGGAATGTCCTGGGACAACTGAGTG
AATTCCAGGTCATTGTTAGGAAGACATCCAGCAAA
GCGGAAGAGATGGTCTTTGAAATTGTTAAGTATCC
GAGAATATTTCGGGGTCATACATTAATCCAGAAAG
GACTAGTCTGTGTCTCCGCAGAAAAATTTGTTAAG
TCACCAGGGAAAGTACAATCTGGAATGGACTATCT
CTTCATTCCGACATTTCTGTCAGTGACTTACTGTC
CAGCTGCAATCAAATTTCAGGTACCTGGCCCCATG
TTGAAAATGAGATCAAGATACACTCAGAGCTTACA
ACTTGAACTAATGATAAGAATCCTGTGTAAGCCCG
ATTCGCCACTTATGAAGGTCCATATCCCTGACAAG
GAAGGAAGAGGATGTCTTGTATCAGTATGGTTGCA
TGTATGCAATATCTTCAAATCAGGAAACAAGAATG
GCAGTGAGTGGCAGGAATACTGGATGAGAAAGTGT
GCTAACATGCAACTTGAAGTGTCGATTGCAGATAT
GTGGGGACCAACTATCATAATTCATGCCAGAGGTC
ACATTCCCAAAAGTGCTAAGTTGTTTTTTGGAAAG
GGTGGATGGAGCTGCCATCCACTTCACGAAGTTGT
TCCAAGTGTCACTAAAACACTATGGTCCGTGGGCT
GTGAGATTACAAAGGCGAAGGCAATAATACAAGAG
AGTAGCATCTCTCTTCTCGTGGAGACTACTGACAT
CATAAGTCCAAAAGTCAAAATTTCATCTAAGCATC
GCCGCTTTGGGAAATCAAATTGGGGTCTGTTCAAG
AAAACCAAATCACTGCCCAACCTGACGGAGCTGGA
ATGACTGACCCCCAATCGAGACTACACCACCTCAA
ACTATAGGTGGGTGGTACCTCAGTGATTAATCTCG
TAAGCACTGATCGTAGGCTACAACACACTAATATT
ATCCAGATTAGAGAGCTTAATTAGCTCTGTATTAA
TAATAACACTACTATTCCAATAACTGGAATCACCA
GCTTGATTTATCTCCAAAATGATTCAAAGAAAACA
AATCATATTAAGACTATCCTAAGCACGAACCCATA
TCGTCCTTCAAATCATGGGTACTATAATTCAATTT
CTGGTGGTCTCCTGTCTATTGGCAGGAGCAGGCAG
TCTTGATCCAGCAGCCCTCATGCAAATCGGTGTCA
TTCCAACAAATGTCCGGCAACTTATGTATTATACT
GAGGCCTCATCAGCATTCATTGTTGTGAAGTTAAT
GCCTACAATTGACTCGCCGATTAGTGGATGTAATA
TAACATCAATTTCAAGCTATAATGCAACAGTGACA
AAACTCCTACAGCCGATCGGTGAGAATTTGGAGAC
GATTAGGAACCAGTTGATTCCAACTCGGAGGAGAC
GCCGGTTTGCAGGGGTGGTGATTGGATTAGCTGCA
TTAGGAGTAGCTACTGCCGCACAGGTCACTGCCGC
AGTAGCACTAGTAAAGGCAAATGAAAATGCTGCGG
CTATACTCAATCTCAAAAATGCAATCCAAAAAACA
AATGCAGCAGTTGCAGATGTGGTCCAGGCCACACA
ATCACTAGGAACaGCAGTTCAAGCAGTTCAAGATC
ACATAAACAGTGTGGTAAGTCCAGCAATTACAGCA
GCCAATTGTAAAGCCCAAGATGCTATCATTGGCTC
AATCCTCAATCTCTATTTGACCGAGTTGACAACTA
TCTTCCACAATCAAATTACAAACCCTGCATTGAGT
CCTATTACAATTCAAGCTTTAAGGATCCTACTAGG
GAGTACCTTGCCGACTGTGGTCGAAAAATCTTTCA
ATACCCAGATAAGTGCGGCTGAGCTTCTCTCATCA
GGGTTATTGACAGGCCAGATTGTGGGATTAGATTT
GACCTATATGCAGATGGTCATAAAAATTGAGCTGC
CAACTTTAACTGTACAACCTGCAACCCAGATCATA
GATCTGGCCACCATTTCTGCATTCATTAACAATCA
AGAAGTCATGGCCCAATTACCAACACGTGTTATTG
TGACTGGCAGCTTGATCCAAGCCTATCCCGCATCG
CAATGCACTATTACACCCAACACTGTGTACTGTAG
GTATAATGATGCCCAAGTACTCTCAGATGATACGA
TGGCTTGCCTCCAAGGTAACTTGACAAGATGCACC
TTCTCTCCAGTGGTTGGGAGCTTTCTCACTCGATT
CGTGCTGTTCGATGGAATAGTTTATGCAAATTGCA
GGTCGATGTTGTGCAAGTGCATGCAGCCTGCTGCT
GTGATCCTACAGCCGAGTTCATCCCCTGTAACTGT
CATTGACATGTACAAATGTGTGAGTCTGCAGCTTG
ACAATCTCAGATTCACCATCACTCAATTGGCCAAT
GTAACCTACAATAGCACCATCAAGCTTGAAACATC
CCAGATCTTGCCTATTGATCCGTTGGATATATCCC
AGAATCTAGCTGCGGTGAATAAGAGTCTAAGTGAT
GCACTACAACACTTAGCACAAAGTGACACATACCT
TTCTGCAATCACATCAGCTACGACTACAAGTGTAT
TATCCATAATAGCAATCTGTCTTGGATCGTTAGGT
TTAATATTAATAATCTTGCTCAGTGTAGTTGTGTG
GAAGTTATTGACCATTGTCGCTGCTAATCGAAATA
GAATGGAGAATTTTGTTTATCATAATTCAGCATTC
CACCACTCACGATCTGATCTCAGTGAGAAAAATCA
ACCTGCAACTCTTGGAACAAGATAAGACAGTCATC
CATTAGTAATTTTTAAGAAAAAAACGATAGGACCG
AAACTAGTATTGAAAGAACCGTCTCGGTCAATCTA
GGTAATCGAGCTGcTACCGTCTCGGAAAGCTCAAA
TCATGCTGCCTGATCCGGAAGATCCGGAAAGCAAA
AAAGCTACAAGGAGAACAGGAAACCTAATTATCTG
CTTCCTATTCATCTTCTTTCCGTTTGTAAACTTCA
TTGTTCCAACTCTAAGACACTTGCTGTCCTAACAC
CTGCTATAGGCTATCCACTGCATCATCTCTTTTTA
AGAAAAAAATAGGCCCGGACGGGTTAGCAACAAGC
GACTGCCGGTGCCAACAACACAATCCACAATCTAC
AGTCGACGCGGCCGCCatgttcgtcttcctggtcc
tgctgcctctggtctccATCcagtgcgtcaatctg
acaactcggactcagctgccacctgcttatactaa
tagcttcaccagaggcgtgtactatcctgacaagg
tgtttagaagctccgtgctgcactctacacaggat
ctgtttctgccattctttagcaacgtgacctggtt
ccacgccatccacgtgagcggcaccaatggcacaa
agcggttcgacaatcccgtgctgccttttaacgat
ggcgtgtacttcgcctctaccgagaagagcaacat
catcagaggctggatctttggcaccacactggact
ccaagacacagtctctgctgatcgtgaacaatgcc
accaacgtggtcatcaaggtgtgcgagttccagtt
ttgtaatgatcccttcctgggcgtgtactatcaca
agaacaataagagctgCatggagtccgagtttaga
gtgtattctagcgccaacaactgcacatttgagta
cgtgagccagcctttcctgatggacctggagggca
agcagggcaatttcaagaacctgagggagttcgtg
tttaagaatatcgacggctacttcaaaatctactc
taagcacacccccatcaacctggtgcgcgacctgc
ctcagggcttcagcgccctggagcccctggtggat
ctgcctatcggcatcaacatcacccggtttcagac
actgctggccctgcacagaagctacctgacacccg
gcgactcctctagcggatggaccgccggcgctgcc
gcctactatgtgggctacctccagccccggacctt
cctgctgaagtacaacgagaatggcaccatcacag
acgcagtggattgcgccctggaTcccctgagcgag
acaaagtgtacactgaagtcctttaccgtggagaa
gggcatctatcagacatccaatttcagggtgcagc
caaccgagtctatcgtgcgctttcctaatatcaca
aacctgtgcccatttggcgaggtgttcaacgcaac
ccgcttcgccagcgtgtacgcctggaataggaagc
ggatcagcaactgcgtggccgactatagcgtgctg
tacaactccgcctctttcagcacctttaagtgcta
tggcgtgtcccccacaaagctgaatgacctgtgct
ttaccaacgtctacgccgattctttcgtgatcagg
ggcgacgaggtgcgccagatcgcccccggccagac
aggcaagatcgcagactacaattataagctgccag
acgatttcaccggctgcgtgatcgcctggaacagc
aacaatctggattccaaagtgggcggcaactacaa
ttatcGgtaccggctgtttagaaagagcaatctga
agcccttcgagagggacatctctacagaaatctac
caggccggcagcaccccttgcaatggcgtggaggg
ctttaactgttatttcccactccagtcctacggct
tccagcccacaaacggcgtgggctatcagccttac
cgcgtggtggtgctgagctttgagctgctgcacgc
cccagcaacagtgtgcggccccaagaagtccacca
atctggtgaagaacaagtgcgtgaacttcaacttc
aacggcctgaccggcacaggcgtgctgaccgagtc
caacaagaagttcctgccatttcagcagttcggca
gggacatcgcagataccacagacgccgtgcgcgac
ccacagaccctggagatcctggacatcacaccctg
ctctttcggcggcgtgagcgtgatcacacccggca
ccaatacaagcaaccaggtggccgtgctgtatcag
gGcgtgaattgtaccgaggtgcccgtggctatcca
cgccgatcagctgaccccaacatggcgggtgtaca
gcaccggctccaacgtcttccagacaagagccgga
tgcctgatcggagcagagcacgtgaacaattccta
tgagtgcgacatcccaatcggcgccggcatctgtg
cctcttaccagacccagacaaactctcccagaaga
gcccggagcgtggcctcccagtctatcatcgccta
taccatgtccctgggcgccgagaacagcgtggcct
actctaacaatagcatcgccatcccaaccaacttc
acaatctctgtgaccacagagatcctgcccgtgtc
catgaccaagacatctgtggactgcacaatgtata
tctgtggcgattctaccgagtgcagcaacctgctg
ctccagtacggcagcttttgtacccagctgaatag
agccctgacaggcatcgccgtggagcaggataaga
acacacaggaggtgttcgcccaggtgaagcaaatc
tacaagaccccccctatcaaggactttggcggctt
caatttttcccagatcctgcctgatccatccaagc
cttctaagcggagctttatcgaggacctgctgttc
aacaaggtgaccctggccgatgccggcttcatcaa
gcagtatggcgattgcctgggcgacatcgcagcca
gggacctgatctgcgcccagaagtttaatggcctg
accgtgctgccacccctgctgacagatgagatgat
cgcacagtacacaagcgccctgctggccggcacca
tcacatccggatggaccttcggcgcaggagccgcc
ctccagatcccctttgccatgcagatggcctatag
gttcaacggcatcggcgtgacccagaatgtgctgt
acgagaaccagaagctgatcgccaatcagtttaac
tccgccatcggcaagatccaggacagcctgtcctc
tacagccagcgccctgggcaagctccaggatgtgg
tgaatcagaacgcccaggccctgaataccctggtg
aagcagctgagcagcaacttcggcgccatctctag
cgtgctgaatgacatcctgagccggctggacaagg
tggaggcagaggtgcagatcgaccggctgatcacc
ggccggctccagagcctccagacctatgtgacaca
gcagctgatcagggccgccgagatcagggccagcg
ccaatctggcagcaaccaagatgtccgagtgcgtg
ctgggccagtctaagagagtggacttttgtggcaa
gggctatcacctgatgtccttccctcagtctgccc
cacacggcgtggtgtttctgcacgtgacctacgtg
cccgcccaggagaagaacttcaccacagcccctgc
catctgccacgatggcaaggcccactttccaaggg
agggcgtgttcgtgtccaacggcacccactggttt
gtgacacagcgcaatttctacgagccccagatcat
caccacagacaacaccttcgtgagcggcaactgtg
acgtggtcatcggcatcgtgaacaataccgtgtat
gatccactccagcccgagctggacagctttaagga
ggagctggataagtatttcaagaatcacacctccc
ctgacgtggatctgggcgacatcagcggcatcaat
gcctccgtggtgaacatccagaaggagatcgaccg
cctgaacgaggtggctaagaatctgaacgagagcc
tgatcgacctccaggagctgggcaagtatgagcag
tacatcaagtggccctggtacatctggctgggctt
catcgccggcctgatcgccatcgtgatggtgacca
tcatgctgtgctgtatgacatcctgctgttcttgc
ctgaagggctgctgtagctgtggctcctgctgtAA
GTTATTGACCATTGTCGTTGCTAATCGAAATAGAA
TGGAGAATTTTGTTTATCATAAATGAacgcgtGCT
AGCCTCCTGCCATACTTCCTACTCACATCATATCT
ATTTTAAAGAAAAAATAGGCCCGAACACTAATCGT
GCCGGCAGTGCCACTGCACACACAACACTACACAT
ACAATACACTACAATGGTTGCAGAAGATGCCCCTG
TTAGGGGCACTTGCCGAGTATTATTTCGAACAACA
ACTTTAATTTTTCTATGCACACTATTAGCATTAAG
CATCTCTATCCTTTATGAGAGTTTAATAACCCAAA
AGCAAATCATGAGCCAAGCAGGCTCAACTGGATCT
AATTCTAGATTAGGAAGTATCACTGATCTTCTTAA
TAATATTCTCTCTGTCGCAAATCAGATTATATATA
ACTCTGCAGTCGCTCTACCTCTACAATTGGACACT
CTTGAATCAACACTCCTTACAGCCATTAAGTCTCT
TCAAACAAGTGACAAGCTAGAACAGAACTGCTCGT
GGGGTGCTGCACTGATTAATGATAATAGATACATT
AATGGCATCAATCAGTTCTATTTCTCAATTGCTGA
GGGTCGCAAGCTGACACTTGGCCCACTTCTTAATA
TACCTAGTTTCATTCCAACTGCCACGACACCAGAG
GGCTGCACCAGGATCCCATCATTCTCGCTCACCAA
GACACACTGGTGTTATACACACAATGTTATCCTGA
ATGGATGCCAGGATCATGTATCCTCAAATCAATTT
GTTTCCATGGGAATCATTGAACCCACTTCTGCCGG
GTTTCCATCCTTTCGAACCCTAAAGACTCTATATC
TCAGCGATGGGGTCAATCGTAAGAGCTGCTCTATC
AGTACAGTTCCGGGGGGTTGTATGATGTACTGTTT
CGTCTCTACTCAACCAGAGAGGGATGACTACTTTT
CTACCGCTCCTCCAGAACAACGAATTATTATAATG
TACTATAATGATACAATCGTGGAGCGCATAATTAA
TCCACCCGGGGTACTAGATGTATGGGCAACATTGA
ACCCAGGAACAGGAAGCGGGGTATATTATTTAGGT
TGGGTACTCTTTCCAATATATGGCGGCGTGATTAA
AGATACGAGTTTATGGAATAATCAAGCAAATAAAT
ACTTTATCCCCCAGATGGTTGCTGCTCTCTGCTCA
CAAAACCAGGCAACTCAAGTCCAAAATGCTAAGTC
ATCATACTATAGCAGCTGGTTTGGCAATCGAATGA
TTCAGTCTGGGATCCTGGCATGTCCTCTTCAACAG
GATCTAACCAATGAGTGTTTAGTTCTGCCCTTTTC
TAATGATCAGGTGCTTATGGGTGCTGAAGGGAGAT
TATACATGTATGGTGACTCGGTGTATTACTACCAA
AGAAGCAATAGTTGGTGGCCTATGACCATGCTGTA
TAAGGTAACCATAACATTCACTAATGGTCAGCCAT
CTGCTATATCAGCTCAGAATGTGCCCACACAGCAG
GTCCCTAGACCTGGGACAGGAAGCTGCTCTGCAAC
AAATAGATGTCCCGGTTTTTGCTTGAAAGGAGTGT
ATGCTGATGCCTGGTTACTGACCAACCCTTCGTCT
ACCAGTACATTTGGATCAGAAGCAACCTTCACTGG
TTCTTATCTCAACGCAGCAACTCAGCGTATCAATC
CGACGATGTATATCGCGAACAACACACAGATCATA
AGCTCACAGCAATTTGGATCAAGCGGTCAAGAAGC
AGCATATGGCCACACAACTTGTTTTAGGGACACAG
GCTCTGTTATGGTATACTGTATCTATATTATTGAA
TTGTCCTCATCTCTCTTAGGACAATTTCAGATTGT
CCCATTTATCCGTCAGGTGACACTATCCTAAAGGC
AGAAGCCTCCAGGTCTGACCCAGCCAATCAAAGCA
TTATACCAGACCATGGAATGCATACCAAACATTAT
TGACACTAATGACACACAAAATTGGTTTTAAGAAA
AACCAAGAGAACAATAGGCCAGAATGGCTGGGTCT
CGGGAGATATTACTCCCTGAAGTCCATCTCAATTC
ACCAATTGTAAAGCATAAGCTATACTATTACATTC
TACTTGGAAACCTCCCAAATGAGATCGACATTGAC
GATTTAGGTCCATTACATAATCAAAATTGGAATCA
AATAGCACATGAAGAGTCTAACTTAGCCCAACGCT
TGGTAAATGTAAGAAATTTTCTAATTACCCACATC
CCTGATCTTAGAAAGGGCCATTGGCAAGAGTATGT
CAATGTAATACTGTGGCCGCGAATTCTTCCCTTGA
TCCCGGATTTTAAAATCAATGACCAATTGCCTCTA
CTCAAAAATTGGGACAAGTTAGTTAAAGAATCATG
TTCAGTAATCAATGCGGGTACTTCCCAGTGCATTC
AGAATCTCAGCTATGGACTGACAGGTCGTGGGAAC
CTCTTTACACGATCACGTGAACTCTCTGGTGACCG
CAGGGATATTGATCTTAAGACGGTTGTGGCAGCAT
GGCATGACTCAGACTGGAAAAGAATAAGTGATTTT
TGGATTATGATCAAATTCCAGATGAGACAATTAAT
TGTTAGGCAAACAGATCATAATGATCCTGATTTAA
TCACGTATATCGAAAATAGAGAAGGCATAATCATC
ATAACCCCTGAACTGGTAGCATTATTTAACACTGA
GAATCATACACTAACATACATGACCTTTGAAATTG
TACTGATGGTTTCAGATATGTACGAAGGTCGTCAC
AACATTTTATCACTATGCACAGTTAGCACTTACCT
GAATCCTCTGAAGAAAAGAATAACATATTTATTGA
GCCTTGTAGATAACTTAGCTTTTCAGATAGGTGAT
GCTGTATATAACATAATTGCTTTGCTAGAATCCTT
TGTATATGCACAGTTGCAAATGTCAGATCCCATCC
CAGAACTCAGAGGACAATTCCATGCATTCGTATGT
TCTGAGATTCTTGATGCACTAAGGGGAACTAATAG
TTTCACCCAGGATGAATCAAGAACTGTGACAACCA
ATTTGATATCCCCATTCCAAGATCTGACCCCAGAT
CTTACGGCTGAATTGCTCTGTATAATGAGGCTTTG
GGGACACCCCATGCTCACCGCCAGTCAAGCTGCGG
GAAAGGTACGCGAGTCCATGTGTGCTGGAAAAGTA
TTAGACTTTCCCACCATTATGAAAACACTAGCCTT
TTTCCATACTATTCTGATCAATGGATACAGGAGGA
AGCATCATGGAGTATGGCCACCCTTAAACTTACCG
GGTAATGCTTCAAAGGGTCTCACGGAACTTATGAA
TGACAATACTGAGATAAGCTATGAATTCACACTTA
AGCATTGGAAGGAAATCTCTCTTATAAAATTCAAG
AAATGTTTTGATGCAGACGCAGGTGAGGAACTCAG
TATATTTATGAAAGATAAAGCAATTAGTGCCCCAA
AACAAGATTGGATGAGTGTGTTTAGAAGAAGCCTA
ATCAAACAGCGCCATCAGCATCATCAGGTCCCCCT
ACCAAATCCATTCAATCGACGGCTATTGCTAAACT
TTCTCGGAGATGACAAATTCGACCCGAATGTGGAG
CTACAGTATGTAACATCAGGTGAGTATCTACATGA
TGACACGTTTTGTGCATCATATTCACTAAAAGAGA
AGGAAATTAAACCTGATGGTCGAATTTTTGCAAAG
TTGACTAAGAGAATGAGATCATGTCAAGTTATAGC
AGAATCTCTTTTAGCGAACCATGCTGGGAAGTTAA
TGAAAGAGAATGGTGTTGTGATGAATCAGCTATCA
TTAACAAAATCACTATTAACAATGAGTCAGATTGG
AATAATATCCGAGAAAGCTAGAAAATCGACTCGAG
ATAACATAAATCAACCTGGTTTCCAGAATATCCAG
AGAAATAAATCACATCACTCCAAGCAAGTCAATCA
GCGAGATCCAAGTGATGACTTTGAATTGGCAGCAT
CTTTTTTAACTACTGATCTCAAAAAATATTGTTTA
CAATGGAGGTACCAGACAATTATCCCATTTGCTCA
ATCATTAAACAGAATGTATGGTTATCCTCATCTCT
TTGAGTGGATTCACTTACGGCTAATGCGTAGTACA
CTTTACGTGGGGGATCCCTTCAACCCACCAGCAGA
TACCAGTCAATTTGATCTAGATAAAGTAATTAATG
GAGATATCTTCATTGTATCACCCAGAGGTGGAATT
GAAGGGCTATGTCAAAAGGCTTGGACAATGATATC
TATCTCTGTGATAATTCTATCTGCCACAGAGTCTG
GCACACGAGTAATGAGTATGGTGCAGGGAGATAAT
CAAGCAATTGCTGTCACCACACGAGTACCAAGGAG
CCTGCCGACTCTTGAGAAAAAGACTATTGCTTTTA
GATCTTGTAATCTATTCTTTGAGAGGTTAAAATGT
AATAATTTTGGATTAGGTCACCATTTGAAAGAACA
AGAGACTATCATTAGTTCTCACTTCTTTGTTTATA
GCAAGAGAATATTCTATCAGGGGAGGATTCTAACG
CAAGCCTTAAAAAATGCTAGTAAGCTCTGCTTGAC
AGCTGATGTCCTAGGAGAATGTACCCAATCATCAT
GTTCTAATCTTGCAACTACTGTCATGAGGTTAACT
GAGAATGGTGTTGAAAAAGATATCTGTTTCTACTT
GAATATCTATATGACCATCAAACAGCTCTCCTATG
ATATCATCTTCCCTCAAGTGTCAATTCCTGGAGAT
CAGATCACATTAGAATACATAAATAATCCACACCT
GGTATCACGATTGGCTCTTCTGCCATCCCAGCTAG
GAGGTCTAAACTACCTGTCATGCAGTAGGCTGTTC
AATCGAAACATAGGCGACCCGGTGGTTTCCGCAGT
TGCAGATCTTAAGAGATTAATTAAATCAGGATGTA
TGGATTACTGGATCCTTTATAACTTATTAGGGAGA
AAACCGGGAAACGGCTCATGGGCTACTTTAGCAGC
TGACCCGTACTCAATCAATATAGAGTATCAATACC
CCCCAACTACAGCTCTTAAGAGGCACACCCAACAA
GCTCTGATGGAACTCAGTACGAATCCAATGTTACG
TGGCATATTCTCTGACAATGCACAGGCAGAAGAAA
ATAATCTTGCTAGATTTCTCCTGGATAGGGAGGTG
ATCTTTCCGCGTGTAGCTCACATCATCATTGAGCA
AACCAGTGTCGGGAGGAGAAAACAGATTCAAGGAT
ATTTGGATTCAACTAGATCGATAATGAGTAAATCA
CTAGAAATTAAGCCCTTGTCCAATAGGAAGCTTAA
TGAAATACTGGATTACAACATCAATTACCTAGCTT
ACAATTTGGCATTACTCAAGAATGCTATTGAACCT
CCGACTTATTTGAAAGCAATGACTCTTGAAACATG
TAGCATCGACATTGCAAGGAGCCTCCGGAAGCTCT
CCTGGGCCCCACTCTTGGGTGGGAGAAATCTTGAA
GGATTAGAGACGCCAGATCCCATTGAAATTACTGC
AGGAGCATTAATTGTTGGATCGGGCTACTGTGAAC
AGTGTGCTGCAGGAGACAATCGATTCACATGGTTT
TTCTTGCCATCTGGTATCGAGATAGGAGGGGATCC
CCGTGATAATCCTCCTATCCGTGTACCGTACATTG
GCTCCAGGACTGATGAGAGGAGGGTAGCCTCAATG
GCATACATCAGGGGTGCCTCGAGTAGCCTAAAAGC
AGTTCTTAGACTGGCGGGAGTGTACATCTGGGCAT
TCGGAGATACTCTGGAGAATTGGATAGATGCACTG
GATTTGTCTCACACTAGAGTTAACATCACACTTGA
ACAGCTGCAATCCCTCACCCCACTTCCAACCTCTG
CCAATCTAACCCATCGGTTGGATGATGGCACAACT
ACCCTAAAGTTTACTCCTGCGAGCTCTTACACCTT
TTCAAGTTTCACTCATATATCAAATGATGAGCAAT
ACCTGACAATTAATGACAAAACTGCAGATTCAAAT
ATAATCTACCAACAGTTAATGATCACTGGACTCGG
AATTTTAGAAACATGGAATAATCCCCCAATCAATA
GAACATTCGAAGAATCTACCCTACATTTGCACACT
GGTGCATCATGTTGTGTCCGACCTGTGGACTCCTG
CATCATCTCAGAAGCATTAACAGTCAAGCCACATA
TTACAGTACCGTACAGCAATAAATTTGTATTTGAT
GAAGACCCGCTATCTGAATATGAGACTGCAAAACT
GGAATCGTTATCATTTCAAGCCCAATTAGGCAACA
TTGATGCTGTAGATATGACAGGTAAATTAACATTA
TTGTCCCAATTCACTGCAAGGCAGATTATTAATGC
AATCACTGGACTCGATGAGTCTGTCTCTCTTACTA
ATGATGCCATTGTTGCATCAGACTATGTCTCCAAT
TGGATTAGTGAATGCATGTATACCAAATTAGATGA
ATTATTTATGTATTGTGGGTGGGAACTACTATTGG
AACTATCCTATCAAATGTATTATCTGAGGGTAGTT
GGGTGGAGTAATATAGTGGATTATTCTTACATGAT
CTTGAGAAGAATCCCGGGTGCAGCATTAAACAATC
TGGCATCTACATTAAGTCATCCAAAACTTTTCCGA
CGAGCTATCAACCTAGATATAGTTGCCCCCTTAAA
TGCTCCTCATTTTGCATCTCTGGACTACATCAAGA
TGAGTGTGGATGCAATACTCTGGGGCTGTAAAAGA
GTCATCAATGTGCTCTCCAATGGAGGGGACTTAGA
ATTAGTTGTGACATCTGAAGATAGCCTTATTCTCA
GTGACCGATCCATGAATCTCATTGCAAGGAAATTA
ACTTTATTATCACTGATTCACCATAATGGTTTGGA
ACTACCAAAGATTAAGGGGTTCTCTCCTGATGAGA
AGTGTTTCGCTTTGACAGAATTTTTGAGGAAAGTG
GTGAACTCAGGGTTGAGTTCAATAGAGAACCTATC
AAATTTTATGTACAATGTGGAGAACCCACGGCTTG
CAGCATTCGCCAGCAACAATTACTACCTGACCAGA
AAATTATTGAATTCAATACGAGATACTGAGTCGGG
TCAAGTAGCAGTCACCTCATATTATGAATCATTAG
AATATATTGATAGTCTTAAGCTAACCCCACATGTG
CCTGGTACCTCATGCATTGAGGATGATAGTCTATG
TACAAATGATTACATAATCTGGATCATAGAGTCTA
ATGCAAACTTGGAGAAGTATCCAATTCCAAATAGC
CCTGAGGATGATTCCAATTTCCATAACTTTAAGTT
GAATGCTCCATCGCACCATACCTTACGCCCATTAG
GGTTGTCATCAACTGCTTGGTATAAGGGTATAAGC
TGTTGCAGGTACCTTGAGCGATTAAAGCTACCACA
AGGTGATCATTTATATATTGCAGAAGGTAGTGGTG
CCAGTATGACAATCATAGAATACCTATTCCCAGGA
AGAAAGATATATTACAATTCTTTATTTAGTAGTGG
TGACAATCCCCCACAAAGAAATTATGCACCAATGC
CTACTCAGTTCATTGAGAGTGTCCCATACAAGCTC
TGGCAAGCACACACAGATCAATATCCCGAGATTTT
TGAGGACTTCATCCCTCTATGGAACGGAAATGCCG
CCATGACTGACATAGGAATGACAGCTTGTGTAGAA
TTCATCATCAATCGAGTCGGCCCAAGGACTTGCAG
TTTAGTACATGTAGATTTGGAATCAAGTGCAAGCT
TAAATCAACAATGCCTGTCAAAGCCGATAATTAAT
GCTATCATCACTGCTACAACTGTTTTGTGCCCTCA
TGGGGTGCTTATTCTGAAATATAGTTGGTTGCCAT
TTACTAGATTTAGTACTTTGATCACTTTCTTATGG
TGCTACTTTGAGAGAATCACTGTTCTTAGGAGCAC
ATATTCTGGTCCAGCTAATCATGAGGTTTATTTAA
TTTGTATCCTTGCCAACAACTTTGCATTCCAGACT
GTCTCGCAGGCAACAGGAATGGCGATGACTTTAAC
CGATCAAGGGTTTACTTTGATATCACCTGAAAGAA
TAAATCAGTATTGGGATGGTCACTTGAAGCAAGAA
CGTATCGTAGCAGAAGCAATTGATAAGGTGGTTCT
AGGAGAAGATGCTCTATTCAATTCGAGTGATAATG
AATTAATTCTCAAATGTGGAGGGACACCAAATGCA
CGGAATCTTATCGATATCGAGCCAGTCGCAACTTT
CATAGAATTTGAACAACTGATCTGCACAATGTTGA
CAACCCACTTGAAGGAAATAATTGATATAACAAGG
TCTGGAACCCAGGATTATGAAAGTTTATTACTCAC
TCCTTACAATTTAGGTCTTCTTGGTAAAATCAGTA
CGATAGTGAGATTATTAACAGAAAGGATTCTAAAT
CATACTATCAGGAATTGGTTGATCCTCCCACCTTC
GCTCCGGATGATCGTGAAGCAGGACTTGGAATTCG
GCATATTCAGGATTACTTCCATCCTCAATTCTGAT
CGGTTCCTGAAGCTTTCTCCAAATAGGAAATACTT
GATTACACAATTAACTGCAGGCTACATTAGGAAAT
TGATTGAGGGGGATTGTAATATCGATCTAACCAGA
CCTATCCAAAAACAAATCTGGAAAGCATTAGGTTG
TGTAGTCTATTGTCACGATCCAGTAGATCAAAGGG
AATCAACAGAGTTTATTGATATAAATATTAATGAA
GAAATAGACCGCGGGATCGATGGCGAGGAAATCTA
AATATATCAAGAATCAGAATTAGTTTAAGAAAAAA
GAAGAGGATTAATCTTGGTTTTCCCCTTGGT.

6. CVL52

The nucleic acid sequence for CVL52 is:

(SEQ ID NO: 66)
ACCAGGGGGAAAACGAAGTGGTGACTCAAATCATCGAAGACCCTCGAGATTACATAGG
TCCGGAACCTATGGCCTTCGTGACCGACCTCGAGTCAGAGTAGTTCAATAAGGACCTATCAAGTTTGG
GCAATTTTTCGTCCCCGACACAAAAATGTCATCCGTGCTTAAAGCATATGAGCGATTCACACTCACTC
AAGAACTGCAAGATCAGAGTGAGGAAGGTACAATCCCACCTACAACACTAAAACCGGTAATCAGGG
TATTTATACTAACCTCTAATAACCCAGAGCTAAGATCCCGGCTTCTTCTATTCTGCCTACGGATTGTTC
TCAGTAATGGTGCAAGGGATTCCCATCGCTTTGGAGCATTACTTACAATGTTTTCGCTACCATCAGCC
ACAATGCTCAATCATGTCAAATTAGCTGACCAGTCACCAGAAGCTGATATCGAAAGGGTAGAGATCG
ATGGCTTTGAGGAGGGATCATTCCGCTTAATCCCCAATGCTCGTTCAGGTATGAGCCGTGGAGAGATC
AATGCCTATGCTGCACTTGCAGAAGATCTACCTGACACACTAAACCATGCAACACCTTTTGTTGATTC
CGAAGTCGAGGGAACTGCATGGGATGAGATTGAGACTTTCTTAGATATGTGTTACAGTGTCCTAATGC
AGGCATGGATAGTGACTTGCAAGTGCATGACTGCGCCAGACCAACCTGCTGCTTCTATTGAGAAACG
CCTGCAAAAATATCGTCAGCAAGGCAGGATCAACCCGAGATATCTCCTGCAACCGGAGGCTCGAAGA
ATAATCCAGAATGTAATCCGAAAGGGAATGGTGGTCAGACATTTCCTCACCTTTGAACTGCAGCTTGC
CCGAGCACAAAGCCTTGTATCAAATAGGTATTATGCTATGGTAGGGGATGTTGGAAAGTATATAGAG
AATTGTGGAATGGGAGGCTTCTTTTTGACACTAAAATATGCATTAGGAACCAGATGGCCCACACTTGC
TTTAGCTGCATTTTCAGGAGAGCTAACAAAGCTAAAGTCCCTCATGGCATTATACCAGACCCTTGGTG
AGCAGGCCCGATATTTGGCCCTATTGGAGTCACCACATTTGATGGATTTTGCTGCAGCAAACTACCCA
CTGCTATATAGCTATGCTATGGGAATAGGCTATGTGTTAGATGTCAACATGAGGAACTACGCTTTCTC
CAGATCATACATGAATAAGACATATTTCCAATTGGGAATGGAAACTGCAAGAAAACAACAGGGTGCA
GTTGACATGAGGATGGCAGAAGATCTCGGTCTAACTCAAGCCGAACGCACCGAGATGGCAAATACAC
TTGCCAAATTGACCACAGCAAATCGAGGGGCAGACACCAGGGGAGGAGTCAACCCGTTCTCATCTAT
CACTGGGACAACTCAGGTGCCCGCTGCAGCAACAGGTGACACATTCGAGAGTTACATGGCAGCGGAT
CGACTGAGGCAGAGATATGCTGATGCAGGCACCCACGATGATGAGATGCCACCATTGGAAGAGGAG
GAAGAGGACGACACATCTGCAGGTCCACGCACTGGACTAACTCTTGAACAAGTGGCCTTGGACATCC
AGAACGCAGCAGTTGGAGCTCCCATCCATACAGATGACCTGAATGCCGCACTGGGTGATCTTGACAT
CTAGACAATTCAGATCCCAATCTTAAATCGACACACCTAATTGACCAGTTAGATGGAACTACAGTGG
ATTCCATAAGGTTCCTGCCTACCATCGGCTTTTAAGAAAAAAATAGGCCCGGACGGGTTAGCAACAA
GCGACTGCCGATGCCAATAACACAATCCACAATCTACAATGGATCCCACTGATCTGAGCTTCTCCCCA
GATGAGATCAATAAGCTCATAGAGACAGGCCTGAATACTGTGGAGTATTTTACTTCCCAACAAGTCA
CAGGAACATCCTCTCTTGGAAAGAATACAATACCACCAGGGGTCACAGGACTACTAACCAATGCTGC
AGAGGCAAAGATCCAAGAGTCAACCAACCATCAGAAGGGTTCAGTTGGTGGGGGCGCAAAACCAAA
GAAACCGCGACCAAAAATTGCCATTGTGCCAGCAGATGACAAAACGGTGCCCGGAAAGCCGATCCC
AAACCCTCTATTAGGTCTGGACTCCACCCCGAGCACCCAAACTGTGCTTGATCTAAGTGGGAAAACAT
TACCATCAGGATCCTATAAGGGGGTTAAACTTGCGAAATTTGGAAAAGAAAATCTGATGACACGGTT
CATCGAGGAACCCAGAGAGAATCCTATCGCAACCAATTCCCCCATCGATTTTAAGAGGGGCAGGGAT
ACCGGCGGGTTCCATAGAAGGGAGTACTCAATCGGATGGGTGGGAGATGAAGTCAAGGTCACTGAGT
GGTGCAATCCATCCTGTTCTCCAATCACCGCTGCAGCAAGGCGATTTGAATGCACTTGTCACCAGTGT
CCAGTCACTTGCTCTGAATGTGAACGAGATACTTAATACAGTGAGAAATTTGGACTCTCGGATGAATC
AACTGGAGACAAAAGTAGATCGCATTCTCTCATCTCAGTCTCTAATCCAGACCATCAAGAATGACAT
AGTTGGACTTAAAGCAGGGATGGCTACTTTAGAAGGAATGATTACAACTGTGAAAATCATGGACCCG
GGAGTTCCCAGTAATGTTACTGTGGAAGATGTACGCAAGAAACTAAGTAACCATGCTGTTGTTGTGCC
AGAATCATTCAATGATAGTTTCTTGACTCAATCTGAAGATGTAATTTCACTTGATGAGTTGGCTCGAC
CAACTGCAACAAGTGTTAAGAAGATTGTCAGGAAGGTTCCTCCTCAGAAGGATCTGACTGGATTGAA
GATCACACTAGAGCAATTGGCAAAGGATTGCATCAGCAAACCGAAGATGAGGGAAGAGTATCTCCTC
AAAATCAACCAGGCTTCCAGTGAGGCTCAGCTAATTGACCTCAAGAAAGCAATCATCCGCAGTGCAA
TTTGATCAAGAAACACCCAATTACACTACACTGGTATGACACTGTACTAACCCTGAGGGTTTTAGAAA
AAACGATTAACGATAAATAAGCCCGAACACTACACACCACCCGAGGCAGCCATGCCATCCATCAGCA
TCCCCGCAGACCCCACCAATCCACGTCAATCAATAAAAGCGTTCCCAATTGTGATCAACCGTGATGG
GGGTGAGAAAGGTCGCTTGGTTAAACAACTACGCACAACCTACTTGAATGACCTAGATACTCATGAG
CCACTGGTGACATTCGTAAATACTTATGGATTCATCTACGAACAGGATCGGGGAAATACCATTGTCGG
AGAGGATCAACATGGGAAGAAAAGAGAGGCTGTGACTGCTGCAATGGTTACCCTTGGATGTGGGCCT
AATCTACCATCATTAGGGAATGTCCTGGGACAACTGAGTGAATTCCAGGTCATTGTTAGGAAGACAT
CCAGCAAAGCGGAAGAGATGGTCTTTGAAATTGTTAAGTATCCGAGAATATTTCGGGGTCATACATT
AATCCAGAAAGGACTAGTCTGTGTCTCCGCAGAAAAATTTGTTAAGTCACCAGGGAAAGTACAATCT
GGAATGGACTATCTCTTCATTCCGACATTTCTGTCAGTGACTTACTGTCCAGCTGCAATCAAATTTCAG
GTACCTGGCCCCATGTTGAAAATGAGATCAAGATACACTCAGAGCTTACAACTTGAACTAATGATAA
GAATCCTGTGTAAGCCCGATTCGCCACTTATGAAGGTCCATATCCCTGACAAGGAAGGAAGAGGATG
TCTTGTATCAGTATGGTTGCATGTATGCAATATCTTCAAATCAGGAAACAAGAATGGCAGTGAGTGGC
AGGAATACTGGATGAGAAAGTGTGCTAACATGCAACTTGAAGTGTCGATTGCAGATATGTGGGGACC
AACTATCATAATTCATGCCAGAGGTCACATTCCCAAAAGTGCTAAGTTGTTTTTTGGAAAGGGTGGAT
GGAGCTGCCATCCACTTCACGAAGTTGTTCCAAGTGTCACTAAAACACTATGGTCCGTGGGCTGTGAG
ATTACAAAGGCGAAGGCAATAATACAAGAGAGTAGCATCTCTCTTCTCGTGGAGACTACTGACATCA
TAAGTCCAAAAGTCAAAATTTCATCTAAGCATCGCCGCTTTGGGAAATCAAATTGGGGTCTGTTCAAG
AAAACCAAATCACTGCCCAACCTGACGGAGCTGGAATGACTGACCCCCAATCGAGACTACACCACCT
CAAACTATAGGTGGGTGGTACCTCAGTGATTAATCTCGTAAGCACTGATCGTAGGCTACAACACACT
AATATTATCCAGATTAGAGAGCTTAATTAGCTCTGTATTAATAATAACACTACTATTCCAATAACTGG
AATCACCAGCTTGATTTATCTCCAAAATGATTCAAAGAAAACAAATCATATTAAGACTATCCTAAGCA
CGAACCCATATCGTCCTTCAAATCATGGGTACTATAATTCAATTTCTGGTGGTCTCCTGTCTATTGGCA
GGAGCAGGCAGTCTTGATCCAGCAGCCCTCATGCAAATCGGTGTCATTCCAACAAATGTCCGGCAAC
TTATGTATTATACTGAGGCCTCATCAGCATTCATTGTTGTGAAGTTAATGCCTACAATTGACTCGCCG
ATTAGTGGATGTAATATAACATCAATTTCAAGCTATAATGCAACAGTGACAAAACTCCTACAGCCGA
TCGGTGAGAATTTGGAGACGATTAGGAACCAGTTGATTCCAACTCGGAGGAGACGCCGGTTTGCAGG
GGTGGTGATTGGATTAGCTGCATTAGGAGTAGCTACTGCCGCACAGGTCACTGCCGCAGTAGCACTA
GTAAAGGCAAATGAAAATGCTGCGGCTATACTCAATCTCAAAAATGCAATCCAAAAAACAAATGCAG
CAGTTGCAGATGTGGTCCAGGCCACACAATCACTAGGAACaGCAGTTCAAGCAGTTCAAGATCACAT
AAACAGTGTGGTAAGTCCAGCAATTACAGCAGCCAATTGTAAAGCCCAAGATGCTATCATTGGCTCA
ATCCTCAATCTCTATTTGACCGAGTTGACAACTATCTTCCACAATCAAATTACAAACCCTGCATTGAG
TCCTATTACAATTCAAGCTTTAAGGATCCTACTAGGGAGTACCTTGCCGACTGTGGTCGAAAAATCTT
TCAATACCCAGATAAGTGCGGCTGAGCTTCTCTCATCAGGGTTATTGACAGGCCAGATTGTGGGATTA
GATTTGACCTATATGCAGATGGTCATAAAAATTGAGCTGCCAACTTTAACTGTACAACCTGCAACCCA
GATCATAGATCTGGCCACCATTTCTGCATTCATTAACAATCAAGAAGTCATGGCCCAATTACCAACAC
GTGTTATTGTGACTGGCAGCTTGATCCAAGCCTATCCCGCATCGCAATGCACTATTACACCCAACACT
GTGTACTGTAGGTATAATGATGCCCAAGTACTCTCAGATGATACGATGGCTTGCCTCCAAGGTAACTT
GACAAGATGCACCTTCTCTCCAGTGGTTGGGAGCTTTCTCACTCGATTCGTGCTGTTCGATGGAATAG
TTTATGCAAATTGCAGGTCGATGTTGTGCAAGTGCATGCAGCCTGCTGCTGTGATCCTACAGCCGAGT
TCATCCCCTGTAACTGTCATTGACATGTACAAATGTGTGAGTCTGCAGCTTGACAATCTCAGATTCAC
CATCACTCAATTGGCCAATGTAACCTACAATAGCACCATCAAGCTTGAAACATCCCAGATCTTGCCTA
TTGATCCGTTGGATATATCCCAGAATCTAGCTGCGGTGAATAAGAGTCTAAGTGATGCACTACAACAC
TTAGCACAAAGTGACACATACCTTTCTGCAATCACATCAGCTACGACTACAAGTGTATTATCCATAAT
AGCAATCTGTCTTGGATCGTTAGGTTTAATATTAATAATCTTGCTCAGTGTAGTTGTGTGGAAGTTATT
GACCATTGTCGCTGCTAATCGAAATAGAATGGAGAATTTTGTTTATCATAATTCAGCATTCCACCACT
CACGATCTGATCTCAGTGAGAAAAATCAACCTGCAACTCTTGGAACAAGATAAGACAGTCATCCATT
AGTAATTTTTAAGAAAAAAACGATAGGACCGAAACTAGTATTGAAAGAACCGTCTCGGTCAATCTAG
GTAATCGAGCTGcTACCGTCTCGGAAAGCTCAAATCGCGCGCCACCatgtcagataacggaccacaga
accagaggaacgcacccaggattactttcggaggaccaagcgatagcaccgggagcaaccagaatgg
agagcggagcggagcaagatccaagcagagacggccccagggcctgccaaacaataccgcatcct
ggttcaccgccctgacacagcacggcaaggaggacctgaagtttccaaggggacagggagtgccta
tcaacaccaatagctcccctgacgatcagatcggctactataggagggcaacaaggagaatcaggg
gaggcgacggcaagatgaaggatctgagcccacgctggtacttctactatctgggaaccggacc
tgaggcaggcctgccatatggcgccaacaaggacggaatcatctgggtggcaaccgagggcgccctg
aacacaccaaaggatcacatcggcacaagaaatcccgccaacaatgcagcaatcgtgctgcagc
tgccacagggaaccacactgcccaagggcttttacgcagagggctctcggggaggcagccaggcatct
agcagatcctctagccggagcagaaactcctctaggaattccaccccaggaagctccaggggcacatcc
cctgcccgcatggcaggaaacggaggcgacgccgccctggccctgctgctgctggatcgcctgaatcag
ctggagtccaagatgtctggcaagggacagcagcagcagggacagaccgtgacaaagaagtccgccgc
cgaggcctctaagaagccaaggcagaagcgcaccgccacaaaggcctacaacgtgacccaggcctt
cggcaggcgcggaccagagcagacacagggcaattttggcgaccaggagctgatcaggcaggga
accgattataagcactggcctcagatcgcccagttcgccccatctgccagcgccttctttggcatgtcta
gaatcggcatggaggtgacccccagcggcacatggctgacctacacaggcgccatcaagctggac
gataaggaccctaacttcaaggatcaggtcatcctgctgaacaagcacatcgacgcctataagaccttt
ccccctacagagcccaagaaggacaagaagaagaaggccgatgagacacaggccctgcctcagaggcagaa
gaagcagcagaccgtgacactgctgccagccgccgatctggacgatttctccaaacagctgcagcagag
catgtccagtgccgactccacccaggcttgaCGTACGACCTGCTATAGGCTATCCACTGCATCATCTC
TTTTTAAGAAAAAAATAGGCCCGGACGGGTTAGCAACAAGCGACTGCCGGTGCCAACAACACAATCC
ACAATCTACAGTCGACGCGGCCGCCACCatgttcgtgttcctggtgctgctgccactggtcagcagccagtgc
gtgaatttcactacccggacccagctgcctcctgcttatacaaacagcttcaccaggggagtgtactac
cccgataaggtgttccgcagctctgtgctgcacagcacccaggacttgtttcttcccttcttcagcaac
gtgacctggttccacgccatccacgtgtccggcaccaacggcaccaaaagattcgccaatcctgtgctgcctt
tcaacgatggcgtgtactttgcctctacagagaagtctaacatcatcagaggctggatcttcggcacaaccc
tggattctaaaacccagagcctgctgatcgtgaacaatgccaccaatgtagtgatcaaggtttgtgagtt
ccagttctgcaacgacccctttctgggcgtttactaccacaagaacaacaaaagctggatggaaagcgag
ttcagggtgtactctagcgccaacaactgtaccttcgagtacgtgagccagccttttctgatggacct
ggagggcaagcagggcaacttcaaaaacctgcgggagttcgtgtttaagaacatcgacggatacttcaag
atttactccaagcacaccccaattaacttggttagaggactgccccaaggcttcagcgccctggaaccc
ctggtggacctgcctatcggcatcaacatcacaagattccaaaccctgcatagaagctatctgacacca
ggcgactctagctctggatggacagccggcgccgctgcttattacgtgggctacctgcagcctagaacctt
cctcctgaaatacaacgagaacggaaccatcaccgacgccgtggactgcgccctggatcctctgagcgagac
aaagtgcaccctgaagtccttcaccgtcgaaaagggcatctaccaaacaagcaacttcagagtgcagcctact
gagagcatcgtgcggtttccaaacatcaccaacctgtgtccttttggcgaggtgttcaacgccacccggttc
gcttccgtgtacgcctggaatagaaagcggatcagcaactgcgtggccgactacagcgtgctgtataat
agcgcctcattttctacttttaaatgttacggcgtgtctcctacaaagctgaacgatctgtgctt
cacaaatgtgtacgccgatagcttcgtgattagaggcgacgaggtgcggcagatcgcccctggcca
gaccggcaacatcgctgactacaactacaagctgcctgacgacttcaccggctgtgtgatcgcat
ggaacagcaataacctggactctaaggtgggcggaaactacaattacctgtacagactgttcag
aaagagcaacctgaagcccttcgagagagatatctccacagaaatctatcaggccggatctacacc
ttgtaatggcgtgaagggcttcaactgctacttccctctgcaatcctacggctttcagcccacatacggcg
tgggctaccagccataccgggtcgtggtgctgagcttcgagctgctgcacgcccctgccacagtgtgc
ggccctaagaagagcaccaatctggtaaagaacaagtgcgtcaacttcaatttcaacggcctgaccg
ggaccggagtgctgaccgaaagcaacaagaaattcctgccgttccagcagttcggcagagatatcgc
cgacaccaccgatgccgtgcgggacccccagacacttgaaatcctggacatcaccccttgcagt
tttggcggcgtgtccgtgatcacacctggaacaaacaccagcaaccaggtggccgtgctgtaccagg
gcgtcaactgcaccgaggtccccgtggccatccacgccgatcagctgacacctacatggcgggtgtact
ctaccggcagcaacgtgttccaaaccagagccggctgcctgatcggcgctgagcacgtgaacaacagc
tacgagtgcgacattcctatcggtgctggcatctgcgcctcttaccagacacagacaaacagc
cccagaagagcgagaagcgtggctagtcagagcatcatcgcctacaccatgagcctgggcgt
ggagaacagcgtggcctactctaacaatagcatcgccatcccaaccaacttcacaatctctgtg
accacagagatcctgcccgtgtccatgaccaagacatctgtggactgcacaatgtatatctgtggcg
attctaccgagtgcagcaacctgctgctccagtacggcagcttttgtacccagctgaataga
gccctgacaggcatcgccgtggagcaggataagaacacacaggaggtgttcgcccaggtgaag
caaatctacaagaccccccctatcaaggactttggcggcttcaatttttcccagatcctgcctgatc
catccaagccttctaagcggagctttatcgaggacctgctgttcaacaaggtgaccctggccg
atgccggcttcatcaagcagtatggcgattgcctgggcgacatcgcagccagggacctgatctgcg
cccagaagtttaatggcctgaccgtgctgccacccctgctgacagatgagatgatcgcacagtac
acaagcgccctgctggccggcaccatcacatccggatggaccttcggcgcaggagccgccctccagat
cccctttgccatgcagatggcctataggttcaacggcatcggcgtgacccagaatgtgctgtacg
agaaccagaagctgatcgccaatcagtttaactccgccatcggcaagatccaggacagcctgtc
ctctacagccagcgccctgggcaagctccaggatgtggtgaatcagaacgcccaggccctgaatac
cctggtgaagcagctgagcagcaacttcggcgccatctctagcgtgctgaatgacatcctgagcc
ggctggacaaggtggaggcagaggtgcagatcgaccggctgatcaccggccggctccagagcctc
cagacctatgtgacacagcagctgatcagggccgccgagatcagggccagcgccaatctggcagc
aaccaagatgtccgagtgcgtgctgggccagtctaagagagtggacttttgtggcaagggctatca
cctgatgtccttccctcagtctgccccacacggcgtggtgtttctgcacgtgacctacgtgcccgccc
aggagaagaacttcaccacagcccctgccatctgccacgatggcaaggcccactttccaagggag
ggcgtgttcgtgtccaacggcacccactggtttgtgacacagcgcaatttctacgagccccaga
tcatcaccacagacaacaccttcgtgagcggcaactgtgacgtggtcatcggcatcgtgaacaata
ccgtgtatgatccactccagcccgagctggacagctttaaggaggagctggataagtatttcaag
aatcacacctcccctgacgtggatctgggcgacatcagcggcatcaatgcctccgtggtgaacatcc
agaaggagatcgaccgcctgaacgaggtggctaagaatctgaacgagagcctgatcgacctccag
gagctgggcaagtatgagcagtacatcaagtggccctggtacatctggctgggcttcatcgccggcctgatcgc
catcgtgatggtgaccatcatgctgtgctgtatgacatcctgctgttcttgcctgaagggctgctgtag
ctgtggctcctgctgtAAGTTATTGACCATTGTCGTTGCTAATCGAAATAGAATGGAGAATTTTGTTTATCATAA
ATGAACGCGTGCTAGCCTCCTGCCATACTTCCTACTCACATCATATCTATTTTAAAGAAAAAATAGGC
CCGAACACTAATCGTGCCGGCAGTGCCACTGCACACACAACACTACACATACAATACACTACAATGG
TTGCAGAAGATGCCCCTGTTAGGGGCACTTGCCGAGTATTATTTCGAACAACAACTTTAATTTTTCTA
TGCACACTATTAGCATTAAGCATCTCTATCCTTTATGAGAGTTTAATAACCCAAAAGCAAATCATGAG
CCAAGCAGGCTCAACTGGATCTAATTCTAGATTAGGAAGTATCACTGATCTTCTTAATAATATTCTCT
CTGTCGCAAATCAGATTATATATAACTCTGCAGTCGCTCTACCTCTACAATTGGACACTCTTGAATCA
ACACTCCTTACAGCCATTAAGTCTCTTCAAACAAGTGACAAGCTAGAACAGAACTGCTCGTGGGGTG
CTGCACTGATTAATGATAATAGATACATTAATGGCATCAATCAGTTCTATTTCTCAATTGCTGAGGGT
CGCAAGCTGACACTTGGCCCACTTCTTAATATACCTAGTTTCATTCCAACTGCCACGACACCAGAGGG
CTGCACCAGGATCCCATCATTCTCGCTCACCAAGACACACTGGTGTTATACACACAATGTTATCCTGA
ATGGATGCCAGGATCATGTATCCTCAAATCAATTTGTTTCCATGGGAATCATTGAACCCACTTCTGCC
GGGTTTCCATCCTTTCGAACCCTAAAGACTCTATATCTCAGCGATGGGGTCAATCGTAAGAGCTGCTC
TATCAGTACAGTTCCGGGGGGTTGTATGATGTACTGTTTCGTCTCTACTCAACCAGAGAGGGATGACT
ACTTTTCTACCGCTCCTCCAGAACAACGAATTATTATAATGTACTATAATGATACAATCGTGGAGCGC
ATAATTAATCCACCCGGGGTACTAGATGTATGGGCAACATTGAACCCAGGAACAGGAAGCGGGGTAT
ATTATTTAGGTTGGGTACTCTTTCCAATATATGGCGGCGTGATTAAAGATACGAGTTTATGGAATAAT
CAAGCAAATAAATACTTTATCCCCCAGATGGTTGCTGCTCTCTGCTCACAAAACCAGGCAACTCAAGT
CCAAAATGCTAAGTCATCATACTATAGCAGCTGGTTTGGCAATCGAATGATTCAGTCTGGGATCCTGG
CATGTCCTCTTCAACAGGATCTAACCAATGAGTGTTTAGTTCTGCCCTTTTCTAATGATCAGGTGCTTA
TGGGTGCTGAAGGGAGATTATACATGTATGGTGACTCGGTGTATTACTACCAAAGAAGCAATAGTTG
GTGGCCTATGACCATGCTGTATAAGGTAACCATAACATTCACTAATGGTCAGCCATCTGCTATATCAG
CTCAGAATGTGCCCACACAGCAGGTCCCTAGACCTGGGACAGGAAGCTGCTCTGCAACAAATAGATG
TCCCGGTTTTTGCTTGAAAGGAGTGTATGCTGATGCCTGGTTACTGACCAACCCTTCGTCTACCAGTA
CATTTGGATCAGAAGCAACCTTCACTGGTTCTTATCTCAACGCAGCAACTCAGCGTATCAATCCGACG
ATGTATATCGCGAACAACACACAGATCATAAGCTCACAGCAATTTGGATCAAGCGGTCAAGAAGCAG
CATATGGCCACACAACTTGTTTTAGGGACACAGGCTCTGTTATGGTATACTGTATCTATATTATTGAA
TTGTCCTCATCTCTCTTAGGACAATTTCAGATTGTCCCATTTATCCGTCAGGTGACACTATCCTAAAGG
CAGAAGCCTCCAGGTCTGACCCAGCCAATCAAAGCATTATACCAGACCATGGAATGCATACCAAACA
TTATTGACACTAATGACACACAAAATTGGTTTTAAGAAAAACCAAGAGAACAATAGGCCAGAATGGC
TGGGTCTCGGGAGATATTACTCCCTGAAGTCCATCTCAATTCACCAATTGTAAAGCATAAGCTATACT
ATTACATTCTACTTGGAAACCTCCCAAATGAGATCGACATTGACGATTTAGGTCCATTACATAATCAA
AATTGGAATCAAATAGCACATGAAGAGTCTAACTTAGCCCAACGCTTGGTAAATGTAAGAAATTTTC
TAATTACCCACATCCCTGATCTTAGAAAGGGCCATTGGCAAGAGTATGTCAATGTAATACTGTGGCCG
CGAATTCTTCCCTTGATCCCGGATTTTAAAATCAATGACCAATTGCCTCTACTCAAAAATTGGGACAA
GTTAGTTAAAGAATCATGTTCAGTAATCAATGCGGGTACTTCCCAGTGCATTCAGAATCTCAGCTATG
GACTGACAGGTCGTGGGAACCTCTTTACACGATCACGTGAACTCTCTGGTGACCGCAGGGATATTGAT
CTTAAGACGGTTGTGGCAGCATGGCATGACTCAGACTGGAAAAGAATAAGTGATTTTTGGATTATGA
TCAAATTCCAGATGAGACAATTAATTGTTAGGCAAACAGATCATAATGATCCTGATTTAATCACGTAT
ATCGAAAATAGAGAAGGCATAATCATCATAACCCCTGAACTGGTAGCATTATTTAACACTGAGAATC
ATACACTAACATACATGACCTTTGAAATTGTACTGATGGTTTCAGATATGTACGAAGGTCGTCACAAC
ATTTTATCACTATGCACAGTTAGCACTTACCTGAATCCTCTGAAGAAAAGAATAACATATTTATTGAG
CCTTGTAGATAACTTAGCTTTTCAGATAGGTGATGCTGTATATAACATAATTGCTTTGCTAGAATCCTT
TGTATATGCACAGTTGCAAATGTCAGATCCCATCCCAGAACTCAGAGGACAATTCCATGCATTCGTAT
GTTCTGAGATTCTTGATGCACTAAGGGGAACTAATAGTTTCACCCAGGATGAATCAAGAACTGTGAC
AACCAATTTGATATCCCCATTCCAAGATCTGACCCCAGATCTTACGGCTGAATTGCTCTGTATAATGA
GGCTTTGGGGACACCCCATGCTCACCGCCAGTCAAGCTGCGGGAAAGGTACGCGAGTCCATGTGTGC
TGGAAAAGTATTAGACTTTCCCACCATTATGAAAACACTAGCCTTTTTCCATACTATTCTGATCAATG
GATACAGGAGGAAGCATCATGGAGTATGGCCACCCTTAAACTTACCGGGTAATGCTTCAAAGGGTCT
CACGGAACTTATGAATGACAATACTGAGATAAGCTATGAATTCACACTTAAGCATTGGAAGGAAATC
TCTCTTATAAAATTCAAGAAATGTTTTGATGCAGACGCAGGTGAGGAACTCAGTATATTTATGAAAGA
TAAAGCAATTAGTGCCCCAAAACAAGATTGGATGAGTGTGTTTAGAAGAAGCCTAATCAAACAGCGC
CATCAGCATCATCAGGTCCCCCTACCAAATCCATTCAATCGACGGCTATTGCTAAACTTTCTCGGAGA
TGACAAATTCGACCCGAATGTGGAGCTACAGTATGTAACATCAGGTGAGTATCTACATGATGACACG
TTTTGTGCATCATATTCACTAAAAGAGAAGGAAATTAAACCTGATGGTCGAATTTTTGCAAAGTTGAC
TAAGAGAATGAGATCATGTCAAGTTATAGCAGAATCTCTTTTAGCGAACCATGCTGGGAAGTTAATG
AAAGAGAATGGTGTTGTGATGAATCAGCTATCATTAACAAAATCACTATTAACAATGAGTCAGATTG
GAATAATATCCGAGAAAGCTAGAAAATCGACTCGAGATAACATAAATCAACCTGGTTTCCAGAATAT
CCAGAGAAATAAATCACATCACTCCAAGCAAGTCAATCAGCGAGATCCAAGTGATGACTTTGAATTG
GCAGCATCTTTTTTAACTACTGATCTCAAAAAATATTGTTTACAATGGAGGTACCAGACAATTATCCC
ATTTGCTCAATCATTAAACAGAATGTATGGTTATCCTCATCTCTTTGAGTGGATTCACTTACGGCTAAT
GCGTAGTACACTTTACGTGGGGGATCCCTTCAACCCACCAGCAGATACCAGTCAATTTGATCTAGATA
AAGTAATTAATGGAGATATCTTCATTGTATCACCCAGAGGTGGAATTGAAGGGCTATGTCAAAAGGC
TTGGACAATGATATCTATCTCTGTGATAATTCTATCTGCCACAGAGTCTGGCACACGAGTAATGAGTA
TGGTGCAGGGAGATAATCAAGCAATTGCTGTCACCACACGAGTACCAAGGAGCCTGCCGACTCTTGA
GAAAAAGACTATTGCTTTTAGATCTTGTAATCTATTCTTTGAGAGGTTAAAATGTAATAATTTTGGATT
AGGTCACCATTTGAAAGAACAAGAGACTATCATTAGTTCTCACTTCTTTGTTTATAGCAAGAGAATAT
TCTATCAGGGGAGGATTCTAACGCAAGCCTTAAAAAATGCTAGTAAGCTCTGCTTGACAGCTGATGTC
CTAGGAGAATGTACCCAATCATCATGTTCTAATCTTGCAACTACTGTCATGAGGTTAACTGAGAATGG
TGTTGAAAAAGATATCTGTTTCTACTTGAATATCTATATGACCATCAAACAGCTCTCCTATGATATCAT
CTTCCCTCAAGTGTCAATTCCTGGAGATCAGATCACATTAGAATACATAAATAATCCACACCTGGTAT
CACGATTGGCTCTTCTGCCATCCCAGCTAGGAGGTCTAAACTACCTGTCATGCAGTAGGCTGTTCAAT
CGAAACATAGGCGACCCGGTGGTTTCCGCAGTTGCAGATCTTAAGAGATTAATTAAATCAGGATGTA
TGGATTACTGGATCCTTTATAACTTATTAGGGAGAAAACCGGGAAACGGCTCATGGGCTACTTTAGCA
GCTGACCCGTACTCAATCAATATAGAGTATCAATACCCCCCAACTACAGCTCTTAAGAGGCACACCC
AACAAGCTCTGATGGAACTCAGTACGAATCCAATGTTACGTGGCATATTCTCTGACAATGCACAGGC
AGAAGAAAATAATCTTGCTAGATTTCTCCTGGATAGGGAGGTGATCTTTCCGCGTGTAGCTCACATCA
TCATTGAGCAAACCAGTGTCGGGAGGAGAAAACAGATTCAAGGATATTTGGATTCAACTAGATCGAT
AATGAGTAAATCACTAGAAATTAAGCCCTTGTCCAATAGGAAGCTTAATGAAATACTGGATTACAAC
ATCAATTACCTAGCTTACAATTTGGCATTACTCAAGAATGCTATTGAACCTCCGACTTATTTGAAAGC
AATGACTCTTGAAACATGTAGCATCGACATTGCAAGGAGCCTCCGGAAGCTCTCCTGGGCCCCACTCT
TGGGTGGGAGAAATCTTGAAGGATTAGAGACGCCAGATCCCATTGAAATTACTGCAGGAGCATTAAT
TGTTGGATCGGGCTACTGTGAACAGTGTGCTGCAGGAGACAATCGATTCACATGGTTTTTCTTGCCAT
CTGGTATCGAGATAGGAGGGGATCCCCGTGATAATCCTCCTATCCGTGTACCGTACATTGGCTCCAGG
ACTGATGAGAGGAGGGTAGCCTCAATGGCATACATCAGGGGTGCCTCGAGTAGCCTAAAAGCAGTTC
TTAGACTGGCGGGAGTGTACATCTGGGCATTCGGAGATACTCTGGAGAATTGGATAGATGCACTGGA
TTTGTCTCACACTAGAGTTAACATCACACTTGAACAGCTGCAATCCCTCACCCCACTTCCAACCTCTG
CCAATCTAACCCATCGGTTGGATGATGGCACAACTACCCTAAAGTTTACTCCTGCGAGCTCTTACACC
TTTTCAAGTTTCACTCATATATCAAATGATGAGCAATACCTGACAATTAATGACAAAACTGCAGATTC
AAATATAATCTACCAACAGTTAATGATCACTGGACTCGGAATTTTAGAAACATGGAATAATCCCCCA
ATCAATAGAACATTCGAAGAATCTACCCTACATTTGCACACTGGTGCATCATGTTGTGTCCGACCTGT
GGACTCCTGCATCATCTCAGAAGCATTAACAGTCAAGCCACATATTACAGTACCGTACAGCAATAAA
TTTGTATTTGATGAAGACCCGCTATCTGAATATGAGACTGCAAAACTGGAATCGTTATCATTTCAAGC
CCAATTAGGCAACATTGATGCTGTAGATATGACAGGTAAATTAACATTATTGTCCCAATTCACTGCAA
GGCAGATTATTAATGCAATCACTGGACTCGATGAGTCTGTCTCTCTTACTAATGATGCCATTGTTGCA
TCAGACTATGTCTCCAATTGGATTAGTGAATGCATGTATACCAAATTAGATGAATTATTTATGTATTG
TGGGTGGGAACTACTATTGGAACTATCCTATCAAATGTATTATCTGAGGGTAGTTGGGTGGAGTAATA
TAGTGGATTATTCTTACATGATCTTGAGAAGAATCCCGGGTGCAGCATTAAACAATCTGGCATCTACA
TTAAGTCATCCAAAACTTTTCCGACGAGCTATCAACCTAGATATAGTTGCCCCCTTAAATGCTCCTCA
TTTTGCATCTCTGGACTACATCAAGATGAGTGTGGATGCAATACTCTGGGGCTGTAAAAGAGTCATCA
ATGTGCTCTCCAATGGAGGGGACTTAGAATTAGTTGTGACATCTGAAGATAGCCTTATTCTCAGTGAC
CGATCCATGAATCTCATTGCAAGGAAATTAACTTTATTATCACTGATTCACCATAATGGTTTGGAACT
ACCAAAGATTAAGGGGTTCTCTCCTGATGAGAAGTGTTTCGCTTTGACAGAATTTTTGAGGAAAGTGG
TGAACTCAGGGTTGAGTTCAATAGAGAACCTATCAAATTTTATGTACAATGTGGAGAACCCACGGCTT
GCAGCATTCGCCAGCAACAATTACTACCTGACCAGAAAATTATTGAATTCAATACGAGATACTGAGT
CGGGTCAAGTAGCAGTCACCTCATATTATGAATCATTAGAATATATTGATAGTCTTAAGCTAACCCCA
CATGTGCCTGGTACCTCATGCATTGAGGATGATAGTCTATGTACAAATGATTACATAATCTGGATCAT
AGAGTCTAATGCAAACTTGGAGAAGTATCCAATTCCAAATAGCCCTGAGGATGATTCCAATTTCCATA
ACTTTAAGTTGAATGCTCCATCGCACCATACCTTACGCCCATTAGGGTTGTCATCAACTGCTTGGTAT
AAGGGTATAAGCTGTTGCAGGTACCTTGAGCGATTAAAGCTACCACAAGGTGATCATTTATATATTGC
AGAAGGTAGTGGTGCCAGTATGACAATCATAGAATACCTATTCCCAGGAAGAAAGATATATTACAAT
TCTTTATTTAGTAGTGGTGACAATCCCCCACAAAGAAATTATGCACCAATGCCTACTCAGTTCATTGA
GAGTGTCCCATACAAGCTCTGGCAAGCACACACAGATCAATATCCCGAGATTTTTGAGGACTTCATCC
CTCTATGGAACGGAAATGCCGCCATGACTGACATAGGAATGACAGCTTGTGTAGAATTCATCATCAA
TCGAGTCGGCCCAAGGACTTGCAGTTTAGTACATGTAGATTTGGAATCAAGTGCAAGCTTAAATCAA
CAATGCCTGTCAAAGCCGATAATTAATGCTATCATCACTGCTACAACTGTTTTGTGCCCTCATGGGGT
GCTTATTCTGAAATATAGTTGGTTGCCATTTACTAGATTTAGTACTTTGATCACTTTCTTATGGTGCTA
CTTTGAGAGAATCACTGTTCTTAGGAGCACATATTCTGGTCCAGCTAATCATGAGGTTTATTTAATTTG
TATCCTTGCCAACAACTTTGCATTCCAGACTGTCTCGCAGGCAACAGGAATGGCGATGACTTTAACCG
ATCAAGGGTTTACTTTGATATCACCTGAAAGAATAAATCAGTATTGGGATGGTCACTTGAAGCAAGA
ACGTATCGTAGCAGAAGCAATTGATAAGGTGGTTCTAGGAGAAGATGCTCTATTCAATTCGAGTGAT
AATGAATTAATTCTCAAATGTGGAGGGACACCAAATGCACGGAATCTTATCGATATCGAGCCAGTCG
CAACTTTCATAGAATTTGAACAACTGATCTGCACAATGTTGACAACCCACTTGAAGGAAATAATTGAT
ATAACAAGGTCTGGAACCCAGGATTATGAAAGTTTATTACTCACTCCTTACAATTTAGGTCTTCTTGG
TAAAATCAGTACGATAGTGAGATTATTAACAGAAAGGATTCTAAATCATACTATCAGGAATTGGTTG
ATCCTCCCACCTTCGCTCCGGATGATCGTGAAGCAGGACTTGGAATTCGGCATATTCAGGATTACTTC
CATCCTCAATTCTGATCGGTTCCTGAAGCTTTCTCCAAATAGGAAATACTTGATTACACAATTAACTG
CAGGCTACATTAGGAAATTGATTGAGGGGGATTGTAATATCGATCTAACCAGACCTATCCAAAAACA
AATCTGGAAAGCATTAGGTTGTGTAGTCTATTGTCACGATCCAGTAGATCAAAGGGAATCAACAGAG
TTTATTGATATAAATATTAATGAAGAAATAGACCGCGGGATCGATGGCGAGGAAATCTAAATATATC
AAGAATCAGAATTAGTTTAAGAAAAAAGAAGAGGATTAATCTTGGTTTTCCCCTTGGT.

7. CVL53

The nucleic acid sequence for CVL53 is:

(SEQ ID NO: 67)
ACCAGGGGGAAAACGAAGTGGTGACTCAAATCATCGAAGACCCTCGAGATTACATAGGTCCGGAACC
TATGGCCTTCGTGACCGACCTCGAGTCAGAGTAGTTCAATAAGGACCTATCAAGTTTGGGCAATTTTTC
GTCCCCGACACAAAAATGTCATCCGTGCTTAAAGCATATGAGCGATTCACACTCACTCAAGAACTGCA
AGATCAGAGTGAGGAAGGTACAATCCCACCTACAACACTAAAACCGGTAATCAGGGTATTTATACTAA
CCTCTAATAACCCAGAGCTAAGATCCCGGCTTCTTCTATTCTGCCTACGGATTGTTCTCAGTAATGGTG
CAAGGGATTCCCATCGCTTTGGAGCATTACTTACAATGTTTTCGCTACCATCAGCCACAATGCTCAATC
ATGTCAAATTAGCTGACCAGTCACCAGAAGCTGATATCGAAAGGGTAGAGATCGATGGCTTTGAGGA
GGGATCATTCCGCTTAATCCCCAATGCTCGTTCAGGTATGAGCCGTGGAGAGATCAATGCCTATGCTG
CACTTGCAGAAGATCTACCTGACACACTAAACCATGCAACACCTTTTGTTGATTCCGAAGTCGAGGGA
ACTGCATGGGATGAGATTGAGACTTTCTTAGATATGTGTTACAGTGTCCTAATGCAGGCATGGATAGT
GACTTGCAAGTGCATGACTGCGCCAGACCAACCTGCTGCTTCTATTGAGAAACGCCTGCAAAAATATC
GTCAGCAAGGCAGGATCAACCCGAGATATCTCCTGCAACCGGAGGCTCGAAGAATAATCCAGAATGT
AATCCGAAAGGGAATGGTGGTCAGACATTTCCTCACCTTTGAACTGCAGCTTGCCCGAGCACAAAGCC
TTGTATCAAATAGGTATTATGCTATGGTAGGGGATGTTGGAAAGTATATAGAGAATTGTGGAATGGGA
GGCTTCTTTTTGACACTAAAATATGCATTAGGAACCAGATGGCCCACACTTGCTTTAGCTGCATTTTCA
GGAGAGCTAACAAAGCTAAAGTCCCTCATGGCATTATACCAGACCCTTGGTGAGCAGGCCCGATATTT
GGCCCTATTGGAGTCACCACATTTGATGGATTTTGCTGCAGCAAACTACCCACTGCTATATAGCTATGC
TATGGGAATAGGCTATGTGTTAGATGTCAACATGAGGAACTACGCTTTCTCCAGATCATACATGAATA
AGACATATTTCCAATTGGGAATGGAAACTGCAAGAAAACAACAGGGTGCAGTTGACATGAGGATGGC
AGAAGATCTCGGTCTAACTCAAGCCGAACGCACCGAGATGGCAAATACACTTGCCAAATTGACCACA
GCAAATCGAGGGGCAGACACCAGGGGAGGAGTCAACCCGTTCTCATCTATCACTGGGACAACTCAGG
TGCCCGCTGCAGCAACAGGTGACACATTCGAGAGTTACATGGCAGCGGATCGACTGAGGCAGAGATA
TGCTGATGCAGGCACCCACGATGATGAGATGCCACCATTGGAAGAGGAGGAAGAGGACGACACATCT
GCAGGTCCACGCACTGGACTAACTCTTGAACAAGTGGCCTTGGACATCCAGAACGCAGCAGTTGGAGC
TCCCATCCATACAGATGACCTGAATGCCGCACTGGGTGATCTTGACATCTAGACAATTCAGATCCCAA
TCTTAAATCGACACACCTAATTGACCAGTTAGATGGAACTACAGTGGATTCCATAAGGTTCCTGCCTAC
CATCGGCTTTTAAGAAAAAAATAGGCCCGGACGGGTTAGCAACAAGCGACTGCCGATGCCAATAACA
CAATCCACAATCTACAATGGATCCCACTGATCTGAGCTTCTCCCCAGATGAGATCAATAAGCTCATAG
AGACAGGCCTGAATACTGTGGAGTATTTTACTTCCCAACAAGTCACAGGAACATCCTCTCTTGGAAAG
AATACAATACCACCAGGGGTCACAGGACTACTAACCAATGCTGCAGAGGCAAAGATCCAAGAGTCAA
CCAACCATCAGAAGGGTTCAGTTGGTGGGGGCGCAAAACCAAAGAAACCGCGACCAAAAATTGCCAT
TGTGCCAGCAGATGACAAAACGGTGCCCGGAAAGCCGATCCCAAACCCTCTATTAGGTCTGGACTCCA
CCCCGAGCACCCAAACTGTGCTTGATCTAAGTGGGAAAACATTACCATCAGGATCCTATAAGGGGGTT
AAACTTGCGAAATTTGGAAAAGAAAATCTGATGACACGGTTCATCGAGGAACCCAGAGAGAATCCTA
TCGCAACCAATTCCCCCATCGATTTTAAGAGGGGCAGGGATACCGGCGGGTTCCATAGAAGGGAGTAC
TCAATCGGATGGGTGGGAGATGAAGTCAAGGTCACTGAGTGGTGCAATCCATCCTGTTCTCCAATCAC
CGCTGCAGCAAGGCGATTTGAATGCACTTGTCACCAGTGTCCAGTCACTTGCTCTGAATGTGAACGAG
ATACTTAATACAGTGAGAAATTTGGACTCTCGGATGAATCAACTGGAGACAAAAGTAGATCGCATTCT
CTCATCTCAGTCTCTAATCCAGACCATCAAGAATGACATAGTTGGACTTAAAGCAGGGATGGCTACTT
TAGAAGGAATGATTACAACTGTGAAAATCATGGACCCGGGAGTTCCCAGTAATGTTACTGTGGAAGAT
GTACGCAAGAAACTAAGTAACCATGCTGTTGTTGTGCCAGAATCATTCAATGATAGTTTCTTGACTCAA
TCTGAAGATGTAATTTCACTTGATGAGTTGGCTCGACCAACTGCAACAAGTGTTAAGAAGATTGTCAG
GAAGGTTCCTCCTCAGAAGGATCTGACTGGATTGAAGATCACACTAGAGCAATTGGCAAAGGATTGCA
TCAGCAAACCGAAGATGAGGGAAGAGTATCTCCTCAAAATCAACCAGGCTTCCAGTGAGGCTCAGCT
AATTGACCTCAAGAAAGCAATCATCCGCAGTGCAATTTGATCAAGAAACACCCAATTACACTACACTG
GTATGACACTGTACTAACCCTGAGGGTTTTAGAAAAAACGATTAACGATAAATAAGCCCGAACACTAC
ACACCACCCGAGGCAGCCATGCCATCCATCAGCATCCCCGCAGACCCCACCAATCCACGTCAATCAAT
AAAAGCGTTCCCAATTGTGATCAACCGTGATGGGGGTGAGAAAGGTCGCTTGGTTAAACAACTACGCA
CAACCTACTTGAATGACCTAGATACTCATGAGCCACTGGTGACATTCGTAAATACTTATGGATTCATCT
ACGAACAGGATCGGGGAAATACCATTGTCGGAGAGGATCAACATGGGAAGAAAAGAGAGGCTGTGA
CTGCTGCAATGGTTACCCTTGGATGTGGGCCTAATCTACCATCATTAGGGAATGTCCTGGGACAACTG
AGTGAATTCCAGGTCATTGTTAGGAAGACATCCAGCAAAGCGGAAGAGATGGTCTTTGAAATTGTTAA
GTATCCGAGAATATTTCGGGGTCATACATTAATCCAGAAAGGACTAGTCTGTGTCTCCGCAGAAAAAT
TTGTTAAGTCACCAGGGAAAGTACAATCTGGAATGGACTATCTCTTCATTCCGACATTTCTGTCAGTGA
CTTACTGTCCAGCTGCAATCAAATTTCAGGTACCTGGCCCCATGTTGAAAATGAGATCAAGATACACT
CAGAGCTTACAACTTGAACTAATGATAAGAATCCTGTGTAAGCCCGATTCGCCACTTATGAAGGTCCA
TATCCCTGACAAGGAAGGAAGAGGATGTCTTGTATCAGTATGGTTGCATGTATGCAATATCTTCAAAT
CAGGAAACAAGAATGGCAGTGAGTGGCAGGAATACTGGATGAGAAAGTGTGCTAACATGCAACTTGA
AGTGTCGATTGCAGATATGTGGGGACCAACTATCATAATTCATGCCAGAGGTCACATTCCCAAAAGTG
CTAAGTTGTTTTTTGGAAAGGGTGGATGGAGCTGCCATCCACTTCACGAAGTTGTTCCAAGTGTCACTA
AAACACTATGGTCCGTGGGCTGTGAGATTACAAAGGCGAAGGCAATAATACAAGAGAGTAGCATCTC
TCTTCTCGTGGAGACTACTGACATCATAAGTCCAAAAGTCAAAATTTCATCTAAGCATCGCCGCTTTGG
GAAATCAAATTGGGGTCTGTTCAAGAAAACCAAATCACTGCCCAACCTGACGGAGCTGGAATGACTG
ACCCCCAATCGAGACTACACCACCTCAAACTATAGGTGGGTGGTACCTCAGTGATTAATCTCGTAAGC
ACTGATCGTAGGCTACAACACACTAATATTATCCAGATTAGAGAGCTTAATTAGCTCTGTATTAATAAT
AACACTACTATTCCAATAACTGGAATCACCAGCTTGATTTATCTCCAAAATGATTCAAAGAAAACAAA
TCATATTAAGACTATCCTAAGCACGAACCCATATCGTCCTTCAAATCATGGGTACTATAATTCAATTTC
TGGTGGTCTCCTGTCTATTGGCAGGAGCAGGCAGTCTTGATCCAGCAGCCCTCATGCAAATCGGTGTC
ATTCCAACAAATGTCCGGCAACTTATGTATTATACTGAGGCCTCATCAGCATTCATTGTTGTGAAGTTA
ATGCCTACAATTGACTCGCCGATTAGTGGATGTAATATAACATCAATTTCAAGCTATAATGCAACAGT
GACAAAACTCCTACAGCCGATCGGTGAGAATTTGGAGACGATTAGGAACCAGTTGATTCCAACTCGGA
GGAGACGCCGGTTTGCAGGGGTGGTGATTGGATTAGCTGCATTAGGAGTAGCTACTGCCGCACAGGTC
ACTGCCGCAGTAGCACTAGTAAAGGCAAATGAAAATGCTGCGGCTATACTCAATCTCAAAAATGCAAT
CCAAAAAACAAATGCAGCAGTTGCAGATGTGGTCCAGGCCACACAATCACTAGGAACaGCAGTTCAAG
CAGTTCAAGATCACATAAACAGTGTGGTAAGTCCAGCAATTACAGCAGCCAATTGTAAAGCCCAAGAT
GCTATCATTGGCTCAATCCTCAATCTCTATTTGACCGAGTTGACAACTATCTTCCACAATCAAATTACA
AACCCTGCATTGAGTCCTATTACAATTCAAGCTTTAAGGATCCTACTAGGGAGTACCTTGCCGACTGTG
GTCGAAAAATCTTTCAATACCCAGATAAGTGCGGCTGAGCTTCTCTCATCAGGGTTATTGACAGGCCA
GATTGTGGGATTAGATTTGACCTATATGCAGATGGTCATAAAAATTGAGCTGCCAACTTTAACTGTAC
AACCTGCAACCCAGATCATAGATCTGGCCACCATTTCTGCATTCATTAACAATCAAGAAGTCATGGCC
CAATTACCAACACGTGTTATTGTGACTGGCAGCTTGATCCAAGCCTATCCCGCATCGCAATGCACTATT
ACACCCAACACTGTGTACTGTAGGTATAATGATGCCCAAGTACTCTCAGATGATACGATGGCTTGCCT
CCAAGGTAACTTGACAAGATGCACCTTCTCTCCAGTGGTTGGGAGCTTTCTCACTCGATTCGTGCTGTT
CGATGGAATAGTTTATGCAAATTGCAGGTCGATGTTGTGCAAGTGCATGCAGCCTGCTGCTGTGATCCT
ACAGCCGAGTTCATCCCCTGTAACTGTCATTGACATGTACAAATGTGTGAGTCTGCAGCTTGACAATCT
CAGATTCACCATCACTCAATTGGCCAATGTAACCTACAATAGCACCATCAAGCTTGAAACATCCCAGA
TCTTGCCTATTGATCCGTTGGATATATCCCAGAATCTAGCTGCGGTGAATAAGAGTCTAAGTGATGCAC
TACAACACTTAGCACAAAGTGACACATACCTTTCTGCAATCACATCAGCTACGACTACAAGTGTATTA
TCCATAATAGCAATCTGTCTTGGATCGTTAGGTTTAATATTAATAATCTTGCTCAGTGTAGTTGTGTGG
AAGTTATTGACCATTGTCGCTGCTAATCGAAATAGAATGGAGAATTTTGTTTATCATAATTCAGCATTC
CACCACTCACGATCTGATCTCAGTGAGAAAAATCAACCTGCAACTCTTGGAACAAGATAAGACAGTCA
TCCATTAGTAATTTTTAAGAAAAAAACGATAGGACCGAAACTAGTATTGAAAGAACCGTCTCGGTCAA
TCTAGGTAATCGAGCTGcTACCGTCTCGGAAAGCTCAAATCGCGCGCCACCatgtcagataacggacca
cagaaccagaggaacgcacccaggattactttcggaggaccaagcgatagcaccgggagcaaccagaat
ggagagcggagcggagcaagatccaagcagagacggccccagggcctgccaaacaataccgcatcctggt
tcaccgccctgacacagcacggcaaggaggacctgaagtttccaaggggacagggagtgcctatcaac
accaatagctcccctgacgatcagatcggctactataggagggcaacaaggagaatcaggggaggcgac
ggcaagatgaaggatctgagcccacgctggtacttctactatctgggaaccggacctgaggcaggcc
tgccatatggcgccaacaaggacggaatcatctgggtggcaaccgagggcgccctgaacacaccaaagg
atcacatcggcacaagaaatcccgccaacaatgcagcaatcgtgctgcagctgccacagggaaccacac
tgcccaagggcttttacgcagagggctctcggggaggcagccaggcatctagcagatcctctagccgg
agcagaaactcctctaggaattccaccccaggaagctccaggggcacatcccctgcccgcatggca
ggaaacggaggcgacgccgccctggccctgctgctgctggatcgcctgaatcagctggagtccaagatg
tctggcaagggacagcagcagcagggacagaccgtgacaaagaagtccgccgccgaggcctctaagaa
gccaaggcagaagcgcaccgccacaaaggcctacaacgtgacccaggccttcggcaggcgcggacc
agagcagacacagggcaattttggcgaccaggagctgatcaggcagggaaccgattataagcactggc
ctcagatcgcccagttcgccccatctgccagcgccttctttggcatgtctagaatcggcatggaggtga
cccccagcggcacatggctgacctacacaggcgccatcaagctggacgataaggaccctaacttcaa
ggatcaggtcatcctgctgaacaagcacatcgacgcctataagacctttccccctacagagcccaaga
aggacaagaagaagaaggccgatgagacacaggccctgcctcagaggcagaagaagcagcagaccgt
gacactgctgccagccgccgatctggacgatttctccaaacagctgcagcagagcat
gtccagtgccgactccacccaggcttgaGCTAGCCTCCTGCCATACTTCCTACTCACA
TCATATCTATTTTAAAGAAAAAATAGGCCCGAACACTAATCGTGCCGGCAGTGCCACTGCACACACAA
CACTACACATACAATACACTACAATGGTTGCAGAAGATGCCCCTGTTAGGGGCACTTGCCGAGTATTA
TTTCGAACAACAACTTTAATTTTTCTATGCACACTATTAGCATTAAGCATCTCTATCCTTTATGAGAGTT
TAATAACCCAAAAGCAAATCATGAGCCAAGCAGGCTCAACTGGATCTAATTCTAGATTAGGAAGTATC
ACTGATCTTCTTAATAATATTCTCTCTGTCGCAAATCAGATTATATATAACTCTGCAGTCGCTCTACCTC
TACAATTGGACACTCTTGAATCAACACTCCTTACAGCCATTAAGTCTCTTCAAACAAGTGACAAGCTA
GAACAGAACTGCTCGTGGGGTGCTGCACTGATTAATGATAATAGATACATTAATGGCATCAATCAGTT
CTATTTCTCAATTGCTGAGGGTCGCAAGCTGACACTTGGCCCACTTCTTAATATACCTAGTTTCATTCC
AACTGCCACGACACCAGAGGGCTGCACCAGGATCCCATCATTCTCGCTCACCAAGACACACTGGTGTT
ATACACACAATGTTATCCTGAATGGATGCCAGGATCATGTATCCTCAAATCAATTTGTTTCCATGGGAA
TCATTGAACCCACTTCTGCCGGGTTTCCATCCTTTCGAACCCTAAAGACTCTATATCTCAGCGATGGGG
TCAATCGTAAGAGCTGCTCTATCAGTACAGTTCCGGGGGGTTGTATGATGTACTGTTTCGTCTCTACTC
AACCAGAGAGGGATGACTACTTTTCTACCGCTCCTCCAGAACAACGAATTATTATAATGTACTATAAT
GATACAATCGTGGAGCGCATAATTAATCCACCCGGGGTACTAGATGTATGGGCAACATTGAACCCAGG
AACAGGAAGCGGGGTATATTATTTAGGTTGGGTACTCTTTCCAATATATGGCGGCGTGATTAAAGATA
CGAGTTTATGGAATAATCAAGCAAATAAATACTTTATCCCCCAGATGGTTGCTGCTCTCTGCTCACAAA
ACCAGGCAACTCAAGTCCAAAATGCTAAGTCATCATACTATAGCAGCTGGTTTGGCAATCGAATGATT
CAGTCTGGGATCCTGGCATGTCCTCTTCAACAGGATCTAACCAATGAGTGTTTAGTTCTGCCCTTTTCT
AATGATCAGGTGCTTATGGGTGCTGAAGGGAGATTATACATGTATGGTGACTCGGTGTATTACTACCA
AAGAAGCAATAGTTGGTGGCCTATGACCATGCTGTATAAGGTAACCATAACATTCACTAATGGTCAGC
CATCTGCTATATCAGCTCAGAATGTGCCCACACAGCAGGTCCCTAGACCTGGGACAGGAAGCTGCTCT
GCAACAAATAGATGTCCCGGTTTTTGCTTGAAAGGAGTGTATGCTGATGCCTGGTTACTGACCAACCCT
TCGTCTACCAGTACATTTGGATCAGAAGCAACCTTCACTGGTTCTTATCTCAACGCAGCAACTCAGCGT
ATCAATCCGACGATGTATATCGCGAACAACACACAGATCATAAGCTCACAGCAATTTGGATCAAGCGG
TCAAGAAGCAGCATATGGCCACACAACTTGTTTTAGGGACACAGGCTCTGTTATGGTATACTGTATCT
ATATTATTGAATTGTCCTCATCTCTCTTAGGACAATTTCAGATTGTCCCATTTATCCGTCAGGTGACACT
ATCCTAAAGGCAGAAGCCTCCAGGTCTGACCCAGCCAATCAAAGCATTATACCAGACCATGGAATGCA
TACCAAACATTATTGACACTAATGACACACAAAATTGGTTTTAAGAAAAACCAAGAGAACAATAGGC
CAGAATGGCTGGGTCTCGGGAGATATTACTCCCTGAAGTCCATCTCAATTCACCAATTGTAAAGCATA
AGCTATACTATTACATTCTACTTGGAAACCTCCCAAATGAGATCGACATTGACGATTTAGGTCCATTAC
ATAATCAAAATTGGAATCAAATAGCACATGAAGAGTCTAACTTAGCCCAACGCTTGGTAAATGTAAGA
AATTTTCTAATTACCCACATCCCTGATCTTAGAAAGGGCCATTGGCAAGAGTATGTCAATGTAATACTG
TGGCCGCGAATTCTTCCCTTGATCCCGGATTTTAAAATCAATGACCAATTGCCTCTACTCAAAAATTGG
GACAAGTTAGTTAAAGAATCATGTTCAGTAATCAATGCGGGTACTTCCCAGTGCATTCAGAATCTCAG
CTATGGACTGACAGGTCGTGGGAACCTCTTTACACGATCACGTGAACTCTCTGGTGACCGCAGGGATA
TTGATCTTAAGACGGTTGTGGCAGCATGGCATGACTCAGACTGGAAAAGAATAAGTGATTTTTGGATT
ATGATCAAATTCCAGATGAGACAATTAATTGTTAGGCAAACAGATCATAATGATCCTGATTTAATCAC
GTATATCGAAAATAGAGAAGGCATAATCATCATAACCCCTGAACTGGTAGCATTATTTAACACTGAGA
ATCATACACTAACATACATGACCTTTGAAATTGTACTGATGGTTTCAGATATGTACGAAGGTCGTCACA
ACATTTTATCACTATGCACAGTTAGCACTTACCTGAATCCTCTGAAGAAAAGAATAACATATTTATTGA
GCCTTGTAGATAACTTAGCTTTTCAGATAGGTGATGCTGTATATAACATAATTGCTTTGCTAGAATCCT
TTGTATATGCACAGTTGCAAATGTCAGATCCCATCCCAGAACTCAGAGGACAATTCCATGCATTCGTAT
GTTCTGAGATTCTTGATGCACTAAGGGGAACTAATAGTTTCACCCAGGATGAATCAAGAACTGTGACA
ACCAATTTGATATCCCCATTCCAAGATCTGACCCCAGATCTTACGGCTGAATTGCTCTGTATAATGAGG
CTTTGGGGACACCCCATGCTCACCGCCAGTCAAGCTGCGGGAAAGGTACGCGAGTCCATGTGTGCTGG
AAAAGTATTAGACTTTCCCACCATTATGAAAACACTAGCCTTTTTCCATACTATTCTGATCAATGGATA
CAGGAGGAAGCATCATGGAGTATGGCCACCCTTAAACTTACCGGGTAATGCTTCAAAGGGTCTCACGG
AACTTATGAATGACAATACTGAGATAAGCTATGAATTCACACTTAAGCATTGGAAGGAAATCTCTCTT
ATAAAATTCAAGAAATGTTTTGATGCAGACGCAGGTGAGGAACTCAGTATATTTATGAAAGATAAAGC
AATTAGTGCCCCAAAACAAGATTGGATGAGTGTGTTTAGAAGAAGCCTAATCAAACAGCGCCATCAGC
ATCATCAGGTCCCCCTACCAAATCCATTCAATCGACGGCTATTGCTAAACTTTCTCGGAGATGACAAAT
TCGACCCGAATGTGGAGCTACAGTATGTAACATCAGGTGAGTATCTACATGATGACACGTTTTGTGCA
TCATATTCACTAAAAGAGAAGGAAATTAAACCTGATGGTCGAATTTTTGCAAAGTTGACTAAGAGAAT
GAGATCATGTCAAGTTATAGCAGAATCTCTTTTAGCGAACCATGCTGGGAAGTTAATGAAAGAGAATG
GTGTTGTGATGAATCAGCTATCATTAACAAAATCACTATTAACAATGAGTCAGATTGGAATAATATCC
GAGAAAGCTAGAAAATCGACTCGAGATAACATAAATCAACCTGGTTTCCAGAATATCCAGAGAAATA
AATCACATCACTCCAAGCAAGTCAATCAGCGAGATCCAAGTGATGACTTTGAATTGGCAGCATCTTTT
TTAACTACTGATCTCAAAAAATATTGTTTACAATGGAGGTACCAGACAATTATCCCATTTGCTCAATCA
TTAAACAGAATGTATGGTTATCCTCATCTCTTTGAGTGGATTCACTTACGGCTAATGCGTAGTACACTT
TACGTGGGGGATCCCTTCAACCCACCAGCAGATACCAGTCAATTTGATCTAGATAAAGTAATTAATGG
AGATATCTTCATTGTATCACCCAGAGGTGGAATTGAAGGGCTATGTCAAAAGGCTTGGACAATGATAT
CTATCTCTGTGATAATTCTATCTGCCACAGAGTCTGGCACACGAGTAATGAGTATGGTGCAGGGAGAT
AATCAAGCAATTGCTGTCACCACACGAGTACCAAGGAGCCTGCCGACTCTTGAGAAAAAGACTATTGC
TTTTAGATCTTGTAATCTATTCTTTGAGAGGTTAAAATGTAATAATTTTGGATTAGGTCACCATTTGAA
AGAACAAGAGACTATCATTAGTTCTCACTTCTTTGTTTATAGCAAGAGAATATTCTATCAGGGGAGGA
TTCTAACGCAAGCCTTAAAAAATGCTAGTAAGCTCTGCTTGACAGCTGATGTCCTAGGAGAATGTACC
CAATCATCATGTTCTAATCTTGCAACTACTGTCATGAGGTTAACTGAGAATGGTGTTGAAAAAGATATC
TGTTTCTACTTGAATATCTATATGACCATCAAACAGCTCTCCTATGATATCATCTTCCCTCAAGTGTCAA
TTCCTGGAGATCAGATCACATTAGAATACATAAATAATCCACACCTGGTATCACGATTGGCTCTTCTGC
CATCCCAGCTAGGAGGTCTAAACTACCTGTCATGCAGTAGGCTGTTCAATCGAAACATAGGCGACCCG
GTGGTTTCCGCAGTTGCAGATCTTAAGAGATTAATTAAATCAGGATGTATGGATTACTGGATCCTTTAT
AACTTATTAGGGAGAAAACCGGGAAACGGCTCATGGGCTACTTTAGCAGCTGACCCGTACTCAATCAA
TATAGAGTATCAATACCCCCCAACTACAGCTCTTAAGAGGCACACCCAACAAGCTCTGATGGAACTCA
GTACGAATCCAATGTTACGTGGCATATTCTCTGACAATGCACAGGCAGAAGAAAATAATCTTGCTAGA
TTTCTCCTGGATAGGGAGGTGATCTTTCCGCGTGTAGCTCACATCATCATTGAGCAAACCAGTGTCGGG
AGGAGAAAACAGATTCAAGGATATTTGGATTCAACTAGATCGATAATGAGTAAATCACTAGAAATTA
AGCCCTTGTCCAATAGGAAGCTTAATGAAATACTGGATTACAACATCAATTACCTAGCTTACAATTTG
GCATTACTCAAGAATGCTATTGAACCTCCGACTTATTTGAAAGCAATGACTCTTGAAACATGTAGCATC
GACATTGCAAGGAGCCTCCGGAAGCTCTCCTGGGCCCCACTCTTGGGTGGGAGAAATCTTGAAGGATT
AGAGACGCCAGATCCCATTGAAATTACTGCAGGAGCATTAATTGTTGGATCGGGCTACTGTGAACAGT
GTGCTGCAGGAGACAATCGATTCACATGGTTTTTCTTGCCATCTGGTATCGAGATAGGAGGGGATCCC
CGTGATAATCCTCCTATCCGTGTACCGTACATTGGCTCCAGGACTGATGAGAGGAGGGTAGCCTCAAT
GGCATACATCAGGGGTGCCTCGAGTAGCCTAAAAGCAGTTCTTAGACTGGCGGGAGTGTACATCTGGG
CATTCGGAGATACTCTGGAGAATTGGATAGATGCACTGGATTTGTCTCACACTAGAGTTAACATCACA
CTTGAACAGCTGCAATCCCTCACCCCACTTCCAACCTCTGCCAATCTAACCCATCGGTTGGATGATGGC
ACAACTACCCTAAAGTTTACTCCTGCGAGCTCTTACACCTTTTCAAGTTTCACTCATATATCAAATGAT
GAGCAATACCTGACAATTAATGACAAAACTGCAGATTCAAATATAATCTACCAACAGTTAATGATCAC
TGGACTCGGAATTTTAGAAACATGGAATAATCCCCCAATCAATAGAACATTCGAAGAATCTACCCTAC
ATTTGCACACTGGTGCATCATGTTGTGTCCGACCTGTGGACTCCTGCATCATCTCAGAAGCATTAACAG
TCAAGCCACATATTACAGTACCGTACAGCAATAAATTTGTATTTGATGAAGACCCGCTATCTGAATAT
GAGACTGCAAAACTGGAATCGTTATCATTTCAAGCCCAATTAGGCAACATTGATGCTGTAGATATGAC
AGGTAAATTAACATTATTGTCCCAATTCACTGCAAGGCAGATTATTAATGCAATCACTGGACTCGATG
AGTCTGTCTCTCTTACTAATGATGCCATTGTTGCATCAGACTATGTCTCCAATTGGATTAGTGAATGCA
TGTATACCAAATTAGATGAATTATTTATGTATTGTGGGTGGGAACTACTATTGGAACTATCCTATCAAA
TGTATTATCTGAGGGTAGTTGGGTGGAGTAATATAGTGGATTATTCTTACATGATCTTGAGAAGAATCC
CGGGTGCAGCATTAAACAATCTGGCATCTACATTAAGTCATCCAAAACTTTTCCGACGAGCTATCAAC
CTAGATATAGTTGCCCCCTTAAATGCTCCTCATTTTGCATCTCTGGACTACATCAAGATGAGTGTGGAT
GCAATACTCTGGGGCTGTAAAAGAGTCATCAATGTGCTCTCCAATGGAGGGGACTTAGAATTAGTTGT
GACATCTGAAGATAGCCTTATTCTCAGTGACCGATCCATGAATCTCATTGCAAGGAAATTAACTTTATT
ATCACTGATTCACCATAATGGTTTGGAACTACCAAAGATTAAGGGGTTCTCTCCTGATGAGAAGTGTTT
CGCTTTGACAGAATTTTTGAGGAAAGTGGTGAACTCAGGGTTGAGTTCAATAGAGAACCTATCAAATT
TTATGTACAATGTGGAGAACCCACGGCTTGCAGCATTCGCCAGCAACAATTACTACCTGACCAGAAAA
TTATTGAATTCAATACGAGATACTGAGTCGGGTCAAGTAGCAGTCACCTCATATTATGAATCATTAGA
ATATATTGATAGTCTTAAGCTAACCCCACATGTGCCTGGTACCTCATGCATTGAGGATGATAGTCTATG
TACAAATGATTACATAATCTGGATCATAGAGTCTAATGCAAACTTGGAGAAGTATCCAATTCCAAATA
GCCCTGAGGATGATTCCAATTTCCATAACTTTAAGTTGAATGCTCCATCGCACCATACCTTACGCCCAT
TAGGGTTGTCATCAACTGCTTGGTATAAGGGTATAAGCTGTTGCAGGTACCTTGAGCGATTAAAGCTA
CCACAAGGTGATCATTTATATATTGCAGAAGGTAGTGGTGCCAGTATGACAATCATAGAATACCTATT
CCCAGGAAGAAAGATATATTACAATTCTTTATTTAGTAGTGGTGACAATCCCCCACAAAGAAATTATG
CACCAATGCCTACTCAGTTCATTGAGAGTGTCCCATACAAGCTCTGGCAAGCACACACAGATCAATAT
CCCGAGATTTTTGAGGACTTCATCCCTCTATGGAACGGAAATGCCGCCATGACTGACATAGGAATGAC
AGCTTGTGTAGAATTCATCATCAATCGAGTCGGCCCAAGGACTTGCAGTTTAGTACATGTAGATTTGG
AATCAAGTGCAAGCTTAAATCAACAATGCCTGTCAAAGCCGATAATTAATGCTATCATCACTGCTACA
ACTGTTTTGTGCCCTCATGGGGTGCTTATTCTGAAATATAGTTGGTTGCCATTTACTAGATTTAGTACTT
TGATCACTTTCTTATGGTGCTACTTTGAGAGAATCACTGTTCTTAGGAGCACATATTCTGGTCCAGCTA
ATCATGAGGTTTATTTAATTTGTATCCTTGCCAACAACTTTGCATTCCAGACTGTCTCGCAGGCAACAG
GAATGGCGATGACTTTAACCGATCAAGGGTTTACTTTGATATCACCTGAAAGAATAAATCAGTATTGG
GATGGTCACTTGAAGCAAGAACGTATCGTAGCAGAAGCAATTGATAAGGTGGTTCTAGGAGAAGATG
CTCTATTCAATTCGAGTGATAATGAATTAATTCTCAAATGTGGAGGGACACCAAATGCACGGAATCTT
ATCGATATCGAGCCAGTCGCAACTTTCATAGAATTTGAACAACTGATCTGCACAATGTTGACAACCCA
CTTGAAGGAAATAATTGATATAACAAGGTCTGGAACCCAGGATTATGAAAGTTTATTACTCACTCCTT
ACAATTTAGGTCTTCTTGGTAAAATCAGTACGATAGTGAGATTATTAACAGAAAGGATTCTAAATCAT
ACTATCAGGAATTGGTTGATCCTCCCACCTTCGCTCCGGATGATCGTGAAGCAGGACTTGGAATTCGG
CATATTCAGGATTACTTCCATCCTCAATTCTGATCGGTTCCTGAAGCTTTCTCCAAATAGGAAATACTT
GATTACACAATTAACTGCAGGCTACATTAGGAAATTGATTGAGGGGGATTGTAATATCGATCTAACCA
GACCTATCCAAAAACAAATCTGGAAAGCATTAGGTTGTGTAGTCTATTGTCACGATCCAGTAGATCAA
AGGGAATCAACAGAGTTTATTGATATAAATATTAATGAAGAAATAGACCGCGGGATCGATGGCGAGG
AAATCTAAATATATCAAGAATCAGAATTAGTTTAAGAAAAAAGAAGAGGATTAATCTTGGTTTTCCCC
TTGGT.

8. CVL54

The nucleic acid sequence for CVL54 is:

(SEQ ID NO: 68)
ACCAGGGGGAAAACGAAGTGGTGACTCAAATCATCGAAGACCCTCGAGATTACATAGGTCCGGAACC
TATGGCCTTCGTGACCGACCTCGAGTCAGAGTAGTTCAATAAGGACCTATCAAGTTTGGGCAATTTTTC
GTCCCCGACACAAAAATGTCATCCGTGCTTAAAGCATATGAGCGATTCACACTCACTCAAGAACTGCA
AGATCAGAGTGAGGAAGGTACAATCCCACCTACAACACTAAAACCGGTAATCAGGGTATTTATACTAA
CCTCTAATAACCCAGAGCTAAGATCCCGGCTTCTTCTATTCTGCCTACGGATTGTTCTCAGTAATGGTG
CAAGGGATTCCCATCGCTTTGGAGCATTACTTACAATGTTTTCGCTACCATCAGCCACAATGCTCAATC
ATGTCAAATTAGCTGACCAGTCACCAGAAGCTGATATCGAAAGGGTAGAGATCGATGGCTTTGAGGA
GGGATCATTCCGCTTAATCCCCAATGCTCGTTCAGGTATGAGCCGTGGAGAGATCAATGCCTATGCTG
CACTTGCAGAAGATCTACCTGACACACTAAACCATGCAACACCTTTTGTTGATTCCGAAGTCGAGGGA
ACTGCATGGGATGAGATTGAGACTTTCTTAGATATGTGTTACAGTGTCCTAATGCAGGCATGGATAGT
GACTTGCAAGTGCATGACTGCGCCAGACCAACCTGCTGCTTCTATTGAGAAACGCCTGCAAAAATATC
GTCAGCAAGGCAGGATCAACCCGAGATATCTCCTGCAACCGGAGGCTCGAAGAATAATCCAGAATGT
AATCCGAAAGGGAATGGTGGTCAGACATTTCCTCACCTTTGAACTGCAGCTTGCCCGAGCACAAAGCC
TTGTATCAAATAGGTATTATGCTATGGTAGGGGATGTTGGAAAGTATATAGAGAATTGTGGAATGGGA
GGCTTCTTTTTGACACTAAAATATGCATTAGGAACCAGATGGCCCACACTTGCTTTAGCTGCATTTTCA
GGAGAGCTAACAAAGCTAAAGTCCCTCATGGCATTATACCAGACCCTTGGTGAGCAGGCCCGATATTT
GGCCCTATTGGAGTCACCACATTTGATGGATTTTGCTGCAGCAAACTACCCACTGCTATATAGCTATGC
TATGGGAATAGGCTATGTGTTAGATGTCAACATGAGGAACTACGCTTTCTCCAGATCATACATGAATA
AGACATATTTCCAATTGGGAATGGAAACTGCAAGAAAACAACAGGGTGCAGTTGACATGAGGATGGC
AGAAGATCTCGGTCTAACTCAAGCCGAACGCACCGAGATGGCAAATACACTTGCCAAATTGACCACA
GCAAATCGAGGGGCAGACACCAGGGGAGGAGTCAACCCGTTCTCATCTATCACTGGGACAACTCAGG
TGCCCGCTGCAGCAACAGGTGACACATTCGAGAGTTACATGGCAGCGGATCGACTGAGGCAGAGATA
TGCTGATGCAGGCACCCACGATGATGAGATGCCACCATTGGAAGAGGAGGAAGAGGACGACACATCT
GCAGGTCCACGCACTGGACTAACTCTTGAACAAGTGGCCTTGGACATCCAGAACGCAGCAGTTGGAGC
TCCCATCCATACAGATGACCTGAATGCCGCACTGGGTGATCTTGACATCTAGACAATTCAGATCCCAA
TCTTAAATCGACACACCTAATTGACCAGTTAGATGGAACTACAGTGGATTCCATAAGGTTCCTGCCTAC
CATCGGCTTTTAAGAAAAAAATAGGCCCGGACGGGTTAGCAACAAGCGACTGCCGATGCCAATAACA
CAATCCACAATCTACAATGGATCCCACTGATCTGAGCTTCTCCCCAGATGAGATCAATAAGCTCATAG
AGACAGGCCTGAATACTGTGGAGTATTTTACTTCCCAACAAGTCACAGGAACATCCTCTCTTGGAAAG
AATACAATACCACCAGGGGTCACAGGACTACTAACCAATGCTGCAGAGGCAAAGATCCAAGAGTCAA
CCAACCATCAGAAGGGTTCAGTTGGTGGGGGCGCAAAACCAAAGAAACCGCGACCAAAAATTGCCAT
TGTGCCAGCAGATGACAAAACGGTGCCCGGAAAGCCGATCCCAAACCCTCTATTAGGTCTGGACTCCA
CCCCGAGCACCCAAACTGTGCTTGATCTAAGTGGGAAAACATTACCATCAGGATCCTATAAGGGGGTT
AAACTTGCGAAATTTGGAAAAGAAAATCTGATGACACGGTTCATCGAGGAACCCAGAGAGAATCCTA
TCGCAACCAATTCCCCCATCGATTTTAAGAGGGGCAGGGATACCGGCGGGTTCCATAGAAGGGAGTAC
TCAATCGGATGGGTGGGAGATGAAGTCAAGGTCACTGAGTGGTGCAATCCATCCTGTTCTCCAATCAC
CGCTGCAGCAAGGCGATTTGAATGCACTTGTCACCAGTGTCCAGTCACTTGCTCTGAATGTGAACGAG
ATACTTAATACAGTGAGAAATTTGGACTCTCGGATGAATCAACTGGAGACAAAAGTAGATCGCATTCT
CTCATCTCAGTCTCTAATCCAGACCATCAAGAATGACATAGTTGGACTTAAAGCAGGGATGGCTACTT
TAGAAGGAATGATTACAACTGTGAAAATCATGGACCCGGGAGTTCCCAGTAATGTTACTGTGGAAGAT
GTACGCAAGAAACTAAGTAACCATGCTGTTGTTGTGCCAGAATCATTCAATGATAGTTTCTTGACTCAA
TCTGAAGATGTAATTTCACTTGATGAGTTGGCTCGACCAACTGCAACAAGTGTTAAGAAGATTGTCAG
GAAGGTTCCTCCTCAGAAGGATCTGACTGGATTGAAGATCACACTAGAGCAATTGGCAAAGGATTGCA
TCAGCAAACCGAAGATGAGGGAAGAGTATCTCCTCAAAATCAACCAGGCTTCCAGTGAGGCTCAGCT
AATTGACCTCAAGAAAGCAATCATCCGCAGTGCAATTTGATCAAGAAACACCCAATTACACTACACTG
GTATGACACTGTACTAACCCTGAGGGTTTTAGAAAAAACGATTAACGATAAATAAGCCCGAACACTAC
ACACCACCCGAGGCAGCCATGCCATCCATCAGCATCCCCGCAGACCCCACCAATCCACGTCAATCAAT
AAAAGCGTTCCCAATTGTGATCAACCGTGATGGGGGTGAGAAAGGTCGCTTGGTTAAACAACTACGCA
CAACCTACTTGAATGACCTAGATACTCATGAGCCACTGGTGACATTCGTAAATACTTATGGATTCATCT
ACGAACAGGATCGGGGAAATACCATTGTCGGAGAGGATCAACATGGGAAGAAAAGAGAGGCTGTGA
CTGCTGCAATGGTTACCCTTGGATGTGGGCCTAATCTACCATCATTAGGGAATGTCCTGGGACAACTG
AGTGAATTCCAGGTCATTGTTAGGAAGACATCCAGCAAAGCGGAAGAGATGGTCTTTGAAATTGTTAA
GTATCCGAGAATATTTCGGGGTCATACATTAATCCAGAAAGGACTAGTCTGTGTCTCCGCAGAAAAAT
TTGTTAAGTCACCAGGGAAAGTACAATCTGGAATGGACTATCTCTTCATTCCGACATTTCTGTCAGTGA
CTTACTGTCCAGCTGCAATCAAATTTCAGGTACCTGGCCCCATGTTGAAAATGAGATCAAGATACACT
CAGAGCTTACAACTTGAACTAATGATAAGAATCCTGTGTAAGCCCGATTCGCCACTTATGAAGGTCCA
TATCCCTGACAAGGAAGGAAGAGGATGTCTTGTATCAGTATGGTTGCATGTATGCAATATCTTCAAAT
CAGGAAACAAGAATGGCAGTGAGTGGCAGGAATACTGGATGAGAAAGTGTGCTAACATGCAACTTGA
AGTGTCGATTGCAGATATGTGGGGACCAACTATCATAATTCATGCCAGAGGTCACATTCCCAAAAGTG
CTAAGTTGTTTTTTGGAAAGGGTGGATGGAGCTGCCATCCACTTCACGAAGTTGTTCCAAGTGTCACTA
AAACACTATGGTCCGTGGGCTGTGAGATTACAAAGGCGAAGGCAATAATACAAGAGAGTAGCATCTC
TCTTCTCGTGGAGACTACTGACATCATAAGTCCAAAAGTCAAAATTTCATCTAAGCATCGCCGCTTTGG
GAAATCAAATTGGGGTCTGTTCAAGAAAACCAAATCACTGCCCAACCTGACGGAGCTGGAATGACTG
ACCCCCAATCGAGACTACACCACCTCAAACTATAGGTGGGTGGTACCTCAGTGATTAATCTCGTAAGC
ACTGATCGTAGGCTACAACACACTAATATTATCCAGATTAGAGAGCTTAATTAGCTCTGTATTAATAAT
AACACTACTATTCCAATAACTGGAATCACCAGCTTGATTTATCTCCAAAATGATTCAAAGAAAACAAA
TCATATTAAGACTATCCTAAGCACGAACCCATATCGTCCTTCAAATCATGGGTACTATAATTCAATTTC
TGGTGGTCTCCTGTCTATTGGCAGGAGCAGGCAGTCTTGATCCAGCAGCCCTCATGCAAATCGGTGTC
ATTCCAACAAATGTCCGGCAACTTATGTATTATACTGAGGCCTCATCAGCATTCATTGTTGTGAAGTTA
ATGCCTACAATTGACTCGCCGATTAGTGGATGTAATATAACATCAATTTCAAGCTATAATGCAACAGT
GACAAAACTCCTACAGCCGATCGGTGAGAATTTGGAGACGATTAGGAACCAGTTGATTCCAACTCGGA
GGAGACGCCGGTTTGCAGGGGTGGTGATTGGATTAGCTGCATTAGGAGTAGCTACTGCCGCACAGGTC
ACTGCCGCAGTAGCACTAGTAAAGGCAAATGAAAATGCTGCGGCTATACTCAATCTCAAAAATGCAAT
CCAAAAAACAAATGCAGCAGTTGCAGATGTGGTCCAGGCCACACAATCACTAGGAACaGCAGTTCAAG
CAGTTCAAGATCACATAAACAGTGTGGTAAGTCCAGCAATTACAGCAGCCAATTGTAAAGCCCAAGAT
GCTATCATTGGCTCAATCCTCAATCTCTATTTGACCGAGTTGACAACTATCTTCCACAATCAAATTACA
AACCCTGCATTGAGTCCTATTACAATTCAAGCTTTAAGGATCCTACTAGGGAGTACCTTGCCGACTGTG
GTCGAAAAATCTTTCAATACCCAGATAAGTGCGGCTGAGCTTCTCTCATCAGGGTTATTGACAGGCCA
GATTGTGGGATTAGATTTGACCTATATGCAGATGGTCATAAAAATTGAGCTGCCAACTTTAACTGTAC
AACCTGCAACCCAGATCATAGATCTGGCCACCATTTCTGCATTCATTAACAATCAAGAAGTCATGGCC
CAATTACCAACACGTGTTATTGTGACTGGCAGCTTGATCCAAGCCTATCCCGCATCGCAATGCACTATT
ACACCCAACACTGTGTACTGTAGGTATAATGATGCCCAAGTACTCTCAGATGATACGATGGCTTGCCT
CCAAGGTAACTTGACAAGATGCACCTTCTCTCCAGTGGTTGGGAGCTTTCTCACTCGATTCGTGCTGTT
CGATGGAATAGTTTATGCAAATTGCAGGTCGATGTTGTGCAAGTGCATGCAGCCTGCTGCTGTGATCCT
ACAGCCGAGTTCATCCCCTGTAACTGTCATTGACATGTACAAATGTGTGAGTCTGCAGCTTGACAATCT
CAGATTCACCATCACTCAATTGGCCAATGTAACCTACAATAGCACCATCAAGCTTGAAACATCCCAGA
TCTTGCCTATTGATCCGTTGGATATATCCCAGAATCTAGCTGCGGTGAATAAGAGTCTAAGTGATGCAC
TACAACACTTAGCACAAAGTGACACATACCTTTCTGCAATCACATCAGCTACGACTACAAGTGTATTA
TCCATAATAGCAATCTGTCTTGGATCGTTAGGTTTAATATTAATAATCTTGCTCAGTGTAGTTGTGTGG
AAGTTATTGACCATTGTCGCTGCTAATCGAAATAGAATGGAGAATTTTGTTTATCATAATTCAGCATTC
CACCACTCACGATCTGATCTCAGTGAGAAAAATCAACCTGCAACTCTTGGAACAAGATAAGACAGTCA
TCCATTAGTAATTTTTAAGAAAAAAACGATAGGACCGAAACTAGTATTGAAAGAACCGTCTCGGTCAA
TCTAGGTAATCGAGCTGcTACCGTCTCGGAAAGCTCAAATCGCGCGCCACCatgtcagataacggacca
cagaaccagaggaacgcacccaggattactttcggaggaccaagcgatagcaccgggagcaaccagaatg
gagagcggagcggagcaagatccaagcagagacggccccagggcctgccaaacaataccgcatcctggttc
accgccctgacacagcacggcaaggaggacctgaagtttccaaggggacagggagtgcctatcaacaccaa
tagctcccctgacgatcagatcggctactataggagggcaacaaggagaatcaggggaggcgacggcaa
gatgaaggatctgagcccacgctggtacttctactatctgggaaccggacctgaggcaggcctgc
catatggcgccaacaaggacggaatcatctgggtggcaaccgagggcgccctgaacacaccaaagga
tcacatcggcacaagaaatcccgccaacaatgcagcaatcgtgctgcagctgccacagggaaccacactg
cccaagggcttttacgcagagggctctcggggaggcagccaggcatctagcagatcctctagccggagc
agaaactcctctaggaattccaccccaggaagctccaggggcacatcccctgcccgcatggcaggaaa
cggaggcgacgccgccctggccctgctgctgctggatcgcctgaatcagctggagtccaagatgtc
tggcaagggacagcagcagcagggacagaccgtgacaaagaagtccgccgccgaggcctctaagaa
gccaaggcagaagcgcaccgccacaaaggcctacaacgtgacccaggccttcggcaggcgcggacca
gagcagacacagggcaattttggcgaccaggagctgatcaggcagggaaccgattataagcact
ggcctcagatcgcccagttcgccccatctgccagcgccttctttggcatgtctagaatcggcatgg
aggtgacccccagcggcacatggctgacctacacaggcgccatcaagctggacgataaggaccct
aacttcaaggatcaggtcatcctgctgaacaagcacatcgacgcctataagacctttccccctacaga
gcccaagaaggacaagaagaagaaggccgatgagacacaggccctgcctcagaggcagaagaagcag
cagaccgtgacactgctgccagccgccgatctggacgatttctccaaacagctgcagcagagcatgtc
cagtgccgactccacccaggcttgaGCTAGCCTCCTGCCATACTTCCTACTCACA
TCATATCTATTTTAAAGAAAAAATAGGCCCGAACACTAATCGTGCCGGCAGTGCCACTGCACACACAA
CACTACACATACAATACACTACAATGGTTGCAGAAGATGCCCCTGTTAGGGGCACTTGCCGAGTATTA
TTTCGAACAACAACTTTAATTTTTCTATGCACACTATTAGCATTAAGCATCTCTATCCTTTATGAGAGTT
TAATAACCCAAAAGCAAATCATGAGCCAAGCAGGCTCAACTGGATCTAATTCTAGATTAGGAAGTATC
ACTGATCTTCTTAATAATATTCTCTCTGTCGCAAATCAGATTATATATAACTCTGCAGTCGCTCTACCTC
TACAATTGGACACTCTTGAATCAACACTCCTTACAGCCATTAAGTCTCTTCAAACAAGTGACAAGCTA
GAACAGAACTGCTCGTGGGGTGCTGCACTGATTAATGATAATAGATACATTAATGGCATCAATCAGTT
CTATTTCTCAATTGCTGAGGGTCGCAAGCTGACACTTGGCCCACTTCTTAATATACCTAGTTTCATTCC
AACTGCCACGACACCAGAGGGCTGCACCAGGATCCCATCATTCTCGCTCACCAAGACACACTGGTGTT
ATACACACAATGTTATCCTGAATGGATGCCAGGATCATGTATCCTCAAATCAATTTGTTTCCATGGGAA
TCATTGAACCCACTTCTGCCGGGTTTCCATCCTTTCGAACCCTAAAGACTCTATATCTCAGCGATGGGG
TCAATCGTAAGAGCTGCTCTATCAGTACAGTTCCGGGGGGTTGTATGATGTACTGTTTCGTCTCTACTC
AACCAGAGAGGGATGACTACTTTTCTACCGCTCCTCCAGAACAACGAATTATTATAATGTACTATAAT
GATACAATCGTGGAGCGCATAATTAATCCACCCGGGGTACTAGATGTATGGGCAACATTGAACCCAGG
AACAGGAAGCGGGGTATATTATTTAGGTTGGGTACTCTTTCCAATATATGGCGGCGTGATTAAAGATA
CGAGTTTATGGAATAATCAAGCAAATAAATACTTTATCCCCCAGATGGTTGCTGCTCTCTGCTCACAAA
ACCAGGCAACTCAAGTCCAAAATGCTAAGTCATCATACTATAGCAGCTGGTTTGGCAATCGAATGATT
CAGTCTGGGATCCTGGCATGTCCTCTTCAACAGGATCTAACCAATGAGTGTTTAGTTCTGCCCTTTTCT
AATGATCAGGTGCTTATGGGTGCTGAAGGGAGATTATACATGTATGGTGACTCGGTGTATTACTACCA
AAGAAGCAATAGTTGGTGGCCTATGACCATGCTGTATAAGGTAACCATAACATTCACTAATGGTCAGC
CATCTGCTATATCAGCTCAGAATGTGCCCACACAGCAGGTCCCTAGACCTGGGACAGGAAGCTGCTCT
GCAACAAATAGATGTCCCGGTTTTTGCTTGAAAGGAGTGTATGCTGATGCCTGGTTACTGACCAACCCT
TCGTCTACCAGTACATTTGGATCAGAAGCAACCTTCACTGGTTCTTATCTCAACGCAGCAACTCAGCGT
ATCAATCCGACGATGTATATCGCGAACAACACACAGATCATAAGCTCACAGCAATTTGGATCAAGCGG
TCAAGAAGCAGCATATGGCCACACAACTTGTTTTAGGGACACAGGCTCTGTTATGGTATACTGTATCT
ATATTATTGAATTGTCCTCATCTCTCTTAGGACAATTTCAGATTGTCCCATTTATCCGTCAGGTGACACT
ATCCTAAACCTGCTATAGGCTATCCACTGCATCATCTCTTTTTAAGAAAAAAATAGGCCCGGACGGGTT
AGCAACAAGCGACTGCCGGTGCCAACAACACAATCCACAATCTACAGTCGACGCGGCCGCCACCatgttc
gtgttcctggtgctgctgccactggtcagcagccagtgcgtgaatttcactacccggacccagctgc
ctcctgcttatacaaacagcttcaccaggggagtgtactaccccgataaggtgttccgcagctctgtgct
gcacagcacccaggacttgtttcttcccttcttcagcaacgtgacctggttccacgccatccacgtgt
ccggcaccaacggcaccaaaagattcgccaatcctgtgctgcctttcaacgatggcgtgtactttgcctc
tacagagaagtctaacatcatcagaggctggatcttcggcacaaccctggattctaaaacccagagcctg
ctgatcgtgaacaatgccaccaatgtagtgatcaaggtttgtgagttccagttctgcaacgaccccttt
ctgggcgtttactaccacaagaacaacaaaagctggatggaaagcgagttcagggtgtactctagcgcc
aacaactgtaccttcgagtacgtgagccagccttttctgatggacctggagggcaagcagggcaacttc
aaaaacctgcgggagttcgtgtttaagaacatcgacggatacttcaagatttactccaagcacaccc
caattaacttggttagaggactgccccaaggcttcagcgccctggaacccctggtggacctgccta
tcggcatcaacatcacaagattccaaaccctgcatagaagctatctgacaccaggcgactctagctc
tggatggacagccggcgccgctgcttattacgtgggctacctgcagcctagaaccttcctcctgaaata
caacgagaacggaaccatcaccgacgccgtggactgcgccctggatcctctgagcgagacaaagtg
caccctgaagtccttcaccgtcgaaaagggcatctaccaaacaagcaacttcagagtgcagcctac
tgagagcatcgtgcggtttccaaacatcaccaacctgtgtccttttggcgaggtgttcaacgccacc
cggttcgcttccgtgtacgcctggaatagaaagcggatcagcaactgcgtggccgactacagcgtgct
gtataatagcgcctcattttctacttttaaatgttacggcgtgtctcctacaaagctgaacgatctgt
gcttcacaaatgtgtacgccgatagcttcgtgattagaggcgacgaggtgcggcagatcgcccctg
gccagaccggcaacatcgctgactacaactacaagctgcctgacgacttcaccggctgtgtgatc
gcatggaacagcaataacctggactctaaggtgggcggaaactacaattacctgtacagactgt
tcagaaagagcaacctgaagcccttcgagagagatatctccacagaaatctatcaggccggatctacac
cttgtaatggcgtgaagggcttcaactgctacttccctctgcaatcctacggctttcagcccacatacgg
cgtgggctaccagccataccgggtcgtggtgctgagcttcgagctgctgcacgcccctgccacagtgtg
cggccctaagaagagcaccaatctggtaaagaacaagtgcgtcaacttcaatttcaacggcctgacc
gggaccggagtgctgaccgaaagcaacaagaaattcctgccgttccagcagttcggcagagatatcgccgac
accaccgatgccgtgcgggacccccagacacttgaaatcctggacatcaccccttgcagttttggcggc
gtgtccgtgatcacacctggaacaaacaccagcaaccaggtggccgtgctgtaccagggcgtca
actgcaccgaggtccccgtggccatccacgccgatcagctgacacctacatggcgggtg
tactctaccggcagcaacgtgttccaaaccagagccggctgcctgatcggcgctga
gcacgtgaacaacagctacgagtgcgacattcctatcggtgctggcatctgcgcctcttaccag
acacagacaaacagccccagaagagcgagaagcgtggctagtcagagcatcatcgcctacaccatg
agcctgggcgtggagaacagcgtggcctactctaacaatagcatcgccatcccaaccaacttcacaa
tctctgtgaccacagagatcctgcccgtgtccatgaccaagacatctgtggactgcacaatgtatatct
gtggcgattctaccgagtgcagcaacctgctgctccagtacggcagcttttgtacccagctgaataga
gccctgacaggcatcgccgtggagcaggataagaacacacaggaggtgttcgcccaggtgaagcaaat
ctacaagaccccccctatcaaggactttggcggcttcaatttttcccagatcctgcctgatccatccaagc
cttctaagcggagctttatcgaggacctgctgttcaacaaggtgaccctggccgatgccggcttcatca
agcagtatggcgattgcctgggcgacatcgcagccagggacctgatctgcgcccagaagtttaatggcctg
accgtgctgccacccctgctgacagatgagatgatcgcacagtacacaagcgccctgctggccggca
ccatcacatccggatggaccttcggcgcaggagccgccctccagatcccctttgccatgcagatggcctat
aggttcaacggcatcggcgtgacccagaatgtgctgtacgagaaccagaagctgatcgccaatcagtttaa
ctccgccatcggcaagatccaggacagcctgtcctctacagccagcgccctgggcaagctccaggatg
tggtgaatcagaacgcccaggccctgaataccctggtgaagcagctgagcagcaacttcggcgccatct
ctagcgtgctgaatgacatcctgagccggctggacaaggtggaggcagaggtgcagatcgaccggctgatca
ccggccggctccagagcctccagacctatgtgacacagcagctgatcagggccgccgagatcagggccag
cgccaatctggcagcaaccaagatgtccgagtgcgtgctgggccagtctaagagagtggacttttgtggc
aagggctatcacctgatgtccttccctcagtctgccccacacggcgtggtgtttctgcacgtgacctac
gtgcccgcccaggagaagaacttcaccacagcccctgccatctgccacgatggcaaggcccactttcca
agggagggcgtgttcgtgtccaacggcacccactggtttgtgacacagcgcaatttctacgagcccc
agatcatcaccacagacaacaccttcgtgagcggcaactgtgacgtggtcatcggcatcgtgaacaat
accgtgtatgatccactccagcccgagctggacagctttaaggaggagctggataagtatttcaagaa
tcacacctcccctgacgtggatctgggcgacatcagcggcatcaatgcctccgtggtgaacatccag
aaggagatcgaccgcctgaacgaggtggctaagaatctgaacgagagcctgatcgacctccaggagc
tgggcaagtatgagcagtacatcaagtggccctggtacatctggctgggcttcatcgccggcctgatcg
ccatcgtgatggtgaccatcatgctgtgctgtatgacatcctgctgttcttgcctgaagggctgctgtagct
gtggctcctgctgtAAGTTATTGACCATTGTCGTTGCTAATCGAAATAGAATGGAGAATTTTGTTTATCATAAA
TGACGTACGAGGCAGAAGCCTCCAGGTCTGACCCAGCCAATCAAAGCATTATACCAGACCATGGAAT
GCATACCAAACATTATTGACACTAATGACACACAAAATTGGTTTTAAGAAAAACCAAGAGAACAATA
GGCCAGAATGGCTGGGTCTCGGGAGATATTACTCCCTGAAGTCCATCTCAATTCACCAATTGTAAAGC
ATAAGCTATACTATTACATTCTACTTGGAAACCTCCCAAATGAGATCGACATTGACGATTTAGGTCCAT
TACATAATCAAAATTGGAATCAAATAGCACATGAAGAGTCTAACTTAGCCCAACGCTTGGTAAATGTA
AGAAATTTTCTAATTACCCACATCCCTGATCTTAGAAAGGGCCATTGGCAAGAGTATGTCAATGTAAT
ACTGTGGCCGCGAATTCTTCCCTTGATCCCGGATTTTAAAATCAATGACCAATTGCCTCTACTCAAAAA
TTGGGACAAGTTAGTTAAAGAATCATGTTCAGTAATCAATGCGGGTACTTCCCAGTGCATTCAGAATC
TCAGCTATGGACTGACAGGTCGTGGGAACCTCTTTACACGATCACGTGAACTCTCTGGTGACCGCAGG
GATATTGATCTTAAGACGGTTGTGGCAGCATGGCATGACTCAGACTGGAAAAGAATAAGTGATTTTTG
GATTATGATCAAATTCCAGATGAGACAATTAATTGTTAGGCAAACAGATCATAATGATCCTGATTTAA
TCACGTATATCGAAAATAGAGAAGGCATAATCATCATAACCCCTGAACTGGTAGCATTATTTAACACT
GAGAATCATACACTAACATACATGACCTTTGAAATTGTACTGATGGTTTCAGATATGTACGAAGGTCG
TCACAACATTTTATCACTATGCACAGTTAGCACTTACCTGAATCCTCTGAAGAAAAGAATAACATATTT
ATTGAGCCTTGTAGATAACTTAGCTTTTCAGATAGGTGATGCTGTATATAACATAATTGCTTTGCTAGA
ATCCTTTGTATATGCACAGTTGCAAATGTCAGATCCCATCCCAGAACTCAGAGGACAATTCCATGCATT
CGTATGTTCTGAGATTCTTGATGCACTAAGGGGAACTAATAGTTTCACCCAGGATGAATCAAGAACTG
TGACAACCAATTTGATATCCCCATTCCAAGATCTGACCCCAGATCTTACGGCTGAATTGCTCTGTATAA
TGAGGCTTTGGGGACACCCCATGCTCACCGCCAGTCAAGCTGCGGGAAAGGTACGCGAGTCCATGTGT
GCTGGAAAAGTATTAGACTTTCCCACCATTATGAAAACACTAGCCTTTTTCCATACTATTCTGATCAAT
GGATACAGGAGGAAGCATCATGGAGTATGGCCACCCTTAAACTTACCGGGTAATGCTTCAAAGGGTCT
CACGGAACTTATGAATGACAATACTGAGATAAGCTATGAATTCACACTTAAGCATTGGAAGGAAATCT
CTCTTATAAAATTCAAGAAATGTTTTGATGCAGACGCAGGTGAGGAACTCAGTATATTTATGAAAGAT
AAAGCAATTAGTGCCCCAAAACAAGATTGGATGAGTGTGTTTAGAAGAAGCCTAATCAAACAGCGCC
ATCAGCATCATCAGGTCCCCCTACCAAATCCATTCAATCGACGGCTATTGCTAAACTTTCTCGGAGATG
ACAAATTCGACCCGAATGTGGAGCTACAGTATGTAACATCAGGTGAGTATCTACATGATGACACGTTT
TGTGCATCATATTCACTAAAAGAGAAGGAAATTAAACCTGATGGTCGAATTTTTGCAAAGTTGACTAA
GAGAATGAGATCATGTCAAGTTATAGCAGAATCTCTTTTAGCGAACCATGCTGGGAAGTTAATGAAAG
AGAATGGTGTTGTGATGAATCAGCTATCATTAACAAAATCACTATTAACAATGAGTCAGATTGGAATA
ATATCCGAGAAAGCTAGAAAATCGACTCGAGATAACATAAATCAACCTGGTTTCCAGAATATCCAGAG
AAATAAATCACATCACTCCAAGCAAGTCAATCAGCGAGATCCAAGTGATGACTTTGAATTGGCAGCAT
CTTTTTTAACTACTGATCTCAAAAAATATTGTTTACAATGGAGGTACCAGACAATTATCCCATTTGCTC
AATCATTAAACAGAATGTATGGTTATCCTCATCTCTTTGAGTGGATTCACTTACGGCTAATGCGTAGTA
CACTTTACGTGGGGGATCCCTTCAACCCACCAGCAGATACCAGTCAATTTGATCTAGATAAAGTAATT
AATGGAGATATCTTCATTGTATCACCCAGAGGTGGAATTGAAGGGCTATGTCAAAAGGCTTGGACAAT
GATATCTATCTCTGTGATAATTCTATCTGCCACAGAGTCTGGCACACGAGTAATGAGTATGGTGCAGG
GAGATAATCAAGCAATTGCTGTCACCACACGAGTACCAAGGAGCCTGCCGACTCTTGAGAAAAAGAC
TATTGCTTTTAGATCTTGTAATCTATTCTTTGAGAGGTTAAAATGTAATAATTTTGGATTAGGTCACCAT
TTGAAAGAACAAGAGACTATCATTAGTTCTCACTTCTTTGTTTATAGCAAGAGAATATTCTATCAGGGG
AGGATTCTAACGCAAGCCTTAAAAAATGCTAGTAAGCTCTGCTTGACAGCTGATGTCCTAGGAGAATG
TACCCAATCATCATGTTCTAATCTTGCAACTACTGTCATGAGGTTAACTGAGAATGGTGTTGAAAAAG
ATATCTGTTTCTACTTGAATATCTATATGACCATCAAACAGCTCTCCTATGATATCATCTTCCCTCAAGT
GTCAATTCCTGGAGATCAGATCACATTAGAATACATAAATAATTCCACACCTGGTATCACGATTGGCTC
TTCTGCCATCCCAGCTAGGAGGTCTAAACTACCTGTCATGCAGTAGGCTGTTCAATCGAAACATAGGC
GACCCGGTGGTTTCCGCAGTTGCAGATCTTAAGAGATTAATTAAATCAGGATGTATGGATTACTGGAT
CCTTTATAACTTATTAGGGAGAAAACCGGGAAACGGCTCATGGGCTACTTTAGCAGCTGACCCGTACT
CAATCAATATAGAGTATCAATACCCCCCAACTACAGCTCTTAAGAGGCACACCCAACAAGCTCTGATG
GAACTCAGTACGAATCCAATGTTACGTGGCATATTCTCTGACAATGCACAGGCAGAAGAAAATAATCT
TGCTAGATTTCTCCTGGATAGGGAGGTGATCTTTCCGCGTGTAGCTCACATCATCATTGAGCAAACCAG
TGTCGGGAGGAGAAAACAGATTCAAGGATATTTGGATTCAACTAGATCGATAATGAGTAAATCACTAG
AAATTAAGCCCTTGTCCAATAGGAAGCTTAATGAAATACTGGATTACAACATCAATTACCTAGCTTAC
AATTTGGCATTACTCAAGAATGCTATTGAACCTCCGACTTATTTGAAAGCAATGACTCTTGAAACATGT
AGCATCGACATTGCAAGGAGCCTCCGGAAGCTCTCCTGGGCCCCACTCTTGGGTGGGAGAAATCTTGA
AGGATTAGAGACGCCAGATCCCATTGAAATTACTGCAGGAGCATTAATTGTTGGATCGGGCTACTGTG
AACAGTGTGCTGCAGGAGACAATCGATTCACATGGTTTTTCTTGCCATCTGGTATCGAGATAGGAGGG
GATCCCCGTGATAATCCTCCTATCCGTGTACCGTACATTGGCTCCAGGACTGATGAGAGGAGGGTAGC
CTCAATGGCATACATCAGGGGTGCCTCGAGTAGCCTAAAAGCAGTTCTTAGACTGGCGGGAGTGTACA
TCTGGGCATTCGGAGATACTCTGGAGAATTGGATAGATGCACTGGATTTGTCTCACACTAGAGTTAAC
ATCACACTTGAACAGCTGCAATCCCTCACCCCACTTCCAACCTCTGCCAATCTAACCCATCGGTTGGAT
GATGGCACAACTACCCTAAAGTTTACTCCTGCGAGCTCTTACACCTTTTCAAGTTTCACTCATATATCA
AATGATGAGCAATACCTGACAATTAATGACAAAACTGCAGATTCAAATATAATCTACCAACAGTTAAT
GATCACTGGACTCGGAATTTTAGAAACATGGAATAATCCCCCAATCAATAGAACATTCGAAGAATCTA
CCCTACATTTGCACACTGGTGCATCATGTTGTGTCCGACCTGTGGACTCCTGCATCATCTCAGAAGCAT
TAACAGTCAAGCCACATATTACAGTACCGTACAGCAATAAATTTGTATTTGATGAAGACCCGCTATCT
GAATATGAGACTGCAAAACTGGAATCGTTATCATTTCAAGCCCAATTAGGCAACATTGATGCTGTAGA
TATGACAGGTAAATTAACATTATTGTCCCAATTCACTGCAAGGCAGATTATTAATGCAATCACTGGACT
CGATGAGTCTGTCTCTCTTACTAATGATGCCATTGTTGCATCAGACTATGTCTCCAATTGGATTAGTGA
ATGCATGTATACCAAATTAGATGAATTATTTATGTATTGTGGGTGGGAACTACTATTGGAACTATCCTA
TCAAATGTATTATCTGAGGGTAGTTGGGTGGAGTAATATAGTGGATTATTCTTACATGATCTTGAGAAG
AATCCCGGGTGCAGCATTAAACAATCTGGCATCTACATTAAGTCATCCAAAACTTTTCCGACGAGCTA
TCAACCTAGATATAGTTGCCCCCTTAAATGCTCCTCATTTTGCATCTCTGGACTACATCAAGATGAGTG
TGGATGCAATACTCTGGGGCTGTAAAAGAGTCATCAATGTGCTCTCCAATGGAGGGGACTTAGAATTA
GTTGTGACATCTGAAGATAGCCTTATTCTCAGTGACCGATCCATGAATCTCATTGCAAGGAAATTAACT
TTATTATCACTGATTCACCATAATGGTTTGGAACTACCAAAGATTAAGGGGTTCTCTCCTGATGAGAAG
TGTTTCGCTTTGACAGAATTTTTGAGGAAAGTGGTGAACTCAGGGTTGAGTTCAATAGAGAACCTATC
AAATTTTATGTACAATGTGGAGAACCCACGGCTTGCAGCATTCGCCAGCAACAATTACTACCTGACCA
GAAAATTATTGAATTCAATACGAGATACTGAGTCGGGTCAAGTAGCAGTCACCTCATATTATGAATCA
TTAGAATATATTGATAGTCTTAAGCTAACCCCACATGTGCCTGGTACCTCATGCATTGAGGATGATAGT
CTATGTACAAATGATTACATAATCTGGATCATAGAGTCTAATGCAAACTTGGAGAAGTATCCAATTCC
AAATAGCCCTGAGGATGATTCCAATTTCCATAACTTTAAGTTGAATGCTCCATCGCACCATACCTTACG
CCCATTAGGGTTGTCATCAACTGCTTGGTATAAGGGTATAAGCTGTTGCAGGTACCTTGAGCGATTAA
AGCTACCACAAGGTGATCATTTATATATTGCAGAAGGTAGTGGTGCCAGTATGACAATCATAGAATAC
CTATTCCCAGGAAGAAAGATATATTACAATTCTTTATTTAGTAGTGGTGACAATCCCCCACAAAGAAA
TTATGCACCAATGCCTACTCAGTTCATTGAGAGTGTCCCATACAAGCTCTGGCAAGCACACACAGATC
AATATCCCGAGATTTTTGAGGACTTCATCCCTCTATGGAACGGAAATGCCGCCATGACTGACATAGGA
ATGACAGCTTGTGTAGAATTCATCATCAATCGAGTCGGCCCAAGGACTTGCAGTTTAGTACATGTAGA
TTTGGAATCAAGTGCAAGCTTAAATCAACAATGCCTGTCAAAGCCGATAATTAATGCTATCATCACTG
CTACAACTGTTTTGTGCCCTCATGGGGTGCTTATTCTGAAATATAGTTGGTTGCCATTTACTAGATTTAG
TACTTTGATCACTTTCTTATGGTGCTACTTTGAGAGAATCACTGTTCTTAGGAGCACATATTCTGGTCCA
GCTAATCATGAGGTTTATTTAATTTGTATCCTTGCCAACAACTTTGCATTCCAGACTGTCTCGCAGGCA
ACAGGAATGGCGATGACTTTAACCGATCAAGGGTTTACTTTGATATCACCTGAAAGAATAAATCAGTA
TTGGGATGGTCACTTGAAGCAAGAACGTATCGTAGCAGAAGCAATTGATAAGGTGGTTCTAGGAGAA
GATGCTCTATTCAATTCGAGTGATAATGAATTAATTCTCAAATGTGGAGGGACACCAAATGCACGGAA
TCTTATCGATATCGAGCCAGTCGCAACTTTCATAGAATTTGAACAACTGATCTGCACAATGTTGACAAC
CCACTTGAAGGAAATAATTGATATAACAAGGTCTGGAACCCAGGATTATGAAAGTTTATTACTCACTC
CTTACAATTTAGGTCTTCTTGGTAAAATCAGTACGATAGTGAGATTATTAACAGAAAGGATTCTAAATC
ATACTATCAGGAATTGGTTGATCCTCCCACCTTCGCTCCGGATGATCGTGAAGCAGGACTTGGAATTCG
GCATATTCAGGATTACTTCCATCCTCAATTCTGATCGGTTCCTGAAGCTTTCTCCAAATAGGAAATACT
TGATTACACAATTAACTGCAGGCTACATTAGGAAATTGATTGAGGGGGATTGTAATATCGATCTAACC
AGACCTATCCAAAAACAAATCTGGAAAGCATTAGGTTGTGTAGTCTATTGTCACGATCCAGTAGATCA
AAGGGAATCAACAGAGTTTATTGATATAAATATTAATGAAGAAATAGACCGCGGGATCGATGGCGAG
GAAATCTAAATATATCAAGAATCAGAATTAGTTTAAGAAAAAAGAAGAGGATTAATCTTGGTTTTCCC
CTTGGT

9. CVL55

The nucleic acid sequence for CVL55 is:

(SEQ ID NO:69)
ACCAGGGGGAAAACGAAGTGGTGACTCAAATCATCGAAGACCCTCGAGATTACATAGGTCCGGAACC
TATGGCCTTCGTGACCGACCTCGAGTCAGAGTAGTTCAATAAGGACCTATCAAGTTTGGGCAATTTTTC
GTCCCCGACACAAAAATGTCATCCGTGCTTAAAGCATATGAGCGATTCACACTCACTCAAGAACTGCA
AGATCAGAGTGAGGAAGGTACAATCCCACCTACAACACTAAAACCGGTAATCAGGGTATTTATACTAA
CCTCTAATAACCCAGAGCTAAGATCCCGGCTTCTTCTATTCTGCCTACGGATTGTTCTCAGTAATGGTG
CAAGGGATTCCCATCGCTTTGGAGCATTACTTACAATGTTTTCGCTACCATCAGCCACAATGCTCAATC
ATGTCAAATTAGCTGACCAGTCACCAGAAGCTGATATCGAAAGGGTAGAGATCGATGGCTTTGAGGA
GGGATCATTCCGCTTAATCCCCAATGCTCGTTCAGGTATGAGCCGTGGAGAGATCAATGCCTATGCTG
CACTTGCAGAAGATCTACCTGACACACTAAACCATGCAACACCTTTTGTTGATTCCGAAGTCGAGGGA
ACTGCATGGGATGAGATTGAGACTTTCTTAGATATGTGTTACAGTGTCCTAATGCAGGCATGGATAGT
GACTTGCAAGTGCATGACTGCGCCAGACCAACCTGCTGCTTCTATTGAGAAACGCCTGCAAAAATATC
GTCAGCAAGGCAGGATCAACCCGAGATATCTCCTGCAACCGGAGGCTCGAAGAATAATCCAGAATGT
AATCCGAAAGGGAATGGTGGTCAGACATTTCCTCACCTTTGAACTGCAGCTTGCCCGAGCACAAAGCC
TTGTATCAAATAGGTATTATGCTATGGTAGGGGATGTTGGAAAGTATATAGAGAATTGTGGAATGGGA
GGCTTCTTTTTGACACTAAAATATGCATTAGGAACCAGATGGCCCACACTTGCTTTAGCTGCATTTTCA
GGAGAGCTAACAAAGCTAAAGTCCCTCATGGCATTATACCAGACCCTTGGTGAGCAGGCCCGATATTT
GGCCCTATTGGAGTCACCACATTTGATGGATTTTGCTGCAGCAAACTACCCACTGCTATATAGCTATGC
TATGGGAATAGGCTATGTGTTAGATGTCAACATGAGGAACTACGCTTTCTCCAGATCATACATGAATA
AGACATATTTCCAATTGGGAATGGAAACTGCAAGAAAACAACAGGGTGCAGTTGACATGAGGATGGC
AGAAGATCTCGGTCTAACTCAAGCCGAACGCACCGAGATGGCAAATACACTTGCCAAATTGACCACA
GCAAATCGAGGGGCAGACACCAGGGGAGGAGTCAACCCGTTCTCATCTATCACTGGGACAACTCAGG
TGCCCGCTGCAGCAACAGGTGACACATTCGAGAGTTACATGGCAGCGGATCGACTGAGGCAGAGATA
TGCTGATGCAGGCACCCACGATGATGAGATGCCACCATTGGAAGAGGAGGAAGAGGACGACACATCT
GCAGGTCCACGCACTGGACTAACTCTTGAACAAGTGGCCTTGGACATCCAGAACGCAGCAGTTGGAGC
TCCCATCCATACAGATGACCTGAATGCCGCACTGGGTGATCTTGACATCTAGACAATTCAGATCCCAA
TCTTAAATCGACACACCTAATTGACCAGTTAGATGGAACTACAGTGGATTCCATAAGGTTCCTGCCTAC
CATCGGCTTTTAAGAAAAAAATAGGCCCGGACGGGTTAGCAACAAGCGACTGCCGATGCCAATAACA
CAATCCACAATCTACAATGGATCCCACTGATCTGAGCTTCTCCCCAGATGAGATCAATAAGCTCATAG
AGACAGGCCTGAATACTGTGGAGTATTTTACTTCCCAACAAGTCACAGGAACATCCTCTCTTGGAAAG
AATACAATACCACCAGGGGTCACAGGACTACTAACCAATGCTGCAGAGGCAAAGATCCAAGAGTCAA
CCAACCATCAGAAGGGTTCAGTTGGTGGGGGCGCAAAACCAAAGAAACCGCGACCAAAAATTGCCAT
TGTGCCAGCAGATGACAAAACGGTGCCCGGAAAGCCGATCCCAAACCCTCTATTAGGTCTGGACTCCA
CCCCGAGCACCCAAACTGTGCTTGATCTAAGTGGGAAAACATTACCATCAGGATCCTATAAGGGGGTT
AAACTTGCGAAATTTGGAAAAGAAAATCTGATGACACGGTTCATCGAGGAACCCAGAGAGAATCCTA
TCGCAACCAATTTCCCCATCGATTTTAAGAGGGGCAGGGATACCGGCGGGTTCCATAGAAGGGAGTAC
TCAATCGGATGGGTGGGAGATGAAGTCAAGGTCACTGAGTGGTGCAATCCATCCTGTTCTCCAATCAC
CGCTGCAGCAAGGCGATTTGAATGCACTTGTCACCAGTGTCCAGTCACTTGCTCTGAATGTGAACGAG
ATACTTAATACAGTGAGAAATTTGGACTCTCGGATGAATCAACTGGAGACAAAAGTAGATCGCATTCT
CTCATCTCAGTCTCTAATCCAGACCATCAAGAATGACATAGTTGGACTTAAAGCAGGGATGGCTACTT
TAGAAGGAATGATTACAACTGTGAAAATCATGGACCCGGGAGTTCCCAGTAATGTTACTGTGGAAGAT
GTACGCAAGAAACTAAGTAACCATGCTGTTGTTGTGCCAGAATCATTCAATGATgccTTCTTGACTCAAT
CTGAAGATGTAATTTCACTTGATGAGTTGGCTCGACCAACTGCAACAAGTGTTAAGAAGATTGTCAGG
AAGGTTCCTCCTCAGAAGGATCTGACTGGATTGAAGATCACACTAGAGCAATTGGCAAAGGATTGCAT
CAGCAAACCGAAGATGAGGGAAGAGTATCTCCTCAAAATCAACCAGGCTTCCAGTGAGGCTCAGCTA
ATTGACCTCAAGAAAGCAATCATCCGCAGTGCAATTTGATCAAGAAACACCCAATTACACTACACTGG
TATGACACTGTACTAACCCTGAGGGTTTTAGAAAAAACGATTAACGATAAATAAGCCCGAACACTACA
CACCACCCGAGGCAGCCATGCCATCCATCAGCATCCCCGCAGACCCCACCAATCCACGTCAATCAATA
AAAGCGTTCCCAATTGTGATCAACCGTGATGGGGGTGAGAAAGGTCGCTTGGTTAAACAACTACGCAC
AACCTACTTGAATGACCTAGATACTCATGAGCCACTGGTGACATTCGTAAATACTTATGGATTCATCTA
CGAACAGGATCGGGGAAATACCATTGTCGGAGAGGATCAACATGGGAAGAAAAGAGAGGCTGTGACT
GCTGCAATGGTTACCCTTGGATGTGGGCCTAATCTACCATCATTAGGGAATGTCCTGGGACAACTGAG
TGAATTCCAGGTCATTGTTAGGAAGACATCCAGCAAAGCGGAAGAGATGGTCTTTGAAATTGTTAAGT
ATCCGAGAATATTTCGGGGTCATACATTAATCCAGAAAGGACTAGTCTGTGTCTCCGCAGAAAAATTT
GTTAAGTCACCAGGGAAAGTACAATCTGGAATGGACTATCTCTTCATTCCGACATTTCTGTCAGTGACT
TACTGTCCAGCTGCAATCAAATTTCAGGTACCTGGCCCCATGTTGAAAATGAGATCAAGATACACTCA
GAGCTTACAACTTGAACTAATGATAAGAATCCTGTGTAAGCCCGATTCGCCACTTATGAAGGTCCATA
TCCCTGACAAGGAAGGAAGAGGATGTCTTGTATCAGTATGGTTGCATGTATGCAATATCTTCAAATCA
GGAAACAAGAATGGCAGTGAGTGGCAGGAATACTGGATGAGAAAGTGTGCTAACATGCAACTTGAAG
TGTCGATTGCAGATATGTGGGGACCAACTATCATAATTCATGCCAGAGGTCACATTCCCAAAAGTGCT
AAGTTGTTTTTTGGAAAGGGTGGATGGAGCTGCCATCCACTTCACGAAGTTGTTCCAAGTGTCACTAA
AACACTATGGTCCGTGGGCTGTGAGATTACAAAGGCGAAGGCAATAATACAAGAGAGTAGCATCTCT
CTTCTCGTGGAGACTACTGACATCATAAGTCCAAAAGTCAAAATTTCATCTAAGCATCGCCGCTTTGGG
AAATCAAATTGGGGTCTGTTCAAGAAAACCAAATCACTGCCCAACCTGACGGAGCTGGAATGACTGAC
CCCCAATCGAGACTACACCACCTCAAACTATAGGTGGGTGGTACCTCAGTGATTAATCTCGTAAGCAC
TGATCGTAGGCTACAACACACTAATATTATCCAGATTAGAGAGCTTAATTAGCTCTGTATTAATAATAA
CACTACTATTCCAATAACTGGAATCACCAGCTTGATTTATCTCCAAAATGATTCAAAGAAAACAAATC
ATATTAAGACTATCCTAAGCACGAACCCATATCGTCCTTCAAATCATGGGTACTATAATTCAATTTCTG
GTGGTCTCCTGTCTATTGGCAGGAGCAGGCAGTCTTGATCCAGCAGCCCTCATGCAAATCGGTGTCATT
CCAACAAATGTCCGGCAACTTATGTATTATACTGAGGCCTCATCAGCATTCATTGTTGTGAAGTTAATG
CCTACAATTGACTCGCCGATTAGTGGATGTAATATAACATCAATTTCAAGCTATAATGCAACAGTGAC
AAAACTCCTACAGCCGATCGGTGAGAATTTGGAGACGATTAGGAACCAGTTGATTCCAACTCGGAGGA
GACGCCGGTTTGCAGGGGTGGTGATTGGATTAGCTGCATTAGGAGTAGCTACTGCCGCACAGGTCACT
GCCGCAGTAGCACTAGTAAAGGCAAATGAAAATGCTGCGGCTATACTCAATCTCAAAAATGCAATCCA
AAAAACAAATGCAGCAGTTGCAGATGTGGTCCAGGCCACACAATCACTAGGAACaGCAGTTCAAGCA
GTTCAAGATCACATAAACAGTGTGGTAAGTCCAGCAATTACAGCAGCCAATTGTAAAGCCCAAGATGC
TATCATTGGCTCAATCCTCAATCTCTATTTGACCGAGTTGACAACTATCTTCCACAATCAAATTACAAA
CCCTGCATTGAGTCCTATTACAATTCAAGCTTTAAGGATCCTACTAGGGAGTACCTTGCCGACTGTGGT
CGAAAAATCTTTCAATACCCAGATAAGTGCGGCTGAGCTTCTCTCATCAGGGTTATTGACAGGCCAGA
TTGTGGGATTAGATTTGACCTATATGCAGATGGTCATAAAAATTGAGCTGCCAACTTTAACTGTACAAC
CTGCAACCCAGATCATAGATCTGGCCACCATTTCTGCATTCATTAACAATCAAGAAGTCATGGCCCAA
TTACCAACACGTGTTATTGTGACTGGCAGCTTGATCCAAGCCTATCCCGCATCGCAATGCACTATTACA
CCCAACACTGTGTACTGTAGGTATAATGATGCCCAAGTACTCTCAGATGATACGATGGCTTGCCTCCA
AGGTAACTTGACAAGATGCACCTTCTCTCCAGTGGTTGGGAGCTTTCTCACTCGATTCGTGCTGTTCGA
TGGAATAGTTTATGCAAATTGCAGGTCGATGTTGTGCAAGTGCATGCAGCCTGCTGCTGTGATCCTACA
GCCGAGTTCATCCCCTGTAACTGTCATTGACATGTACAAATGTGTGAGTCTGCAGCTTGACAATCTCAG
ATTCACCATCACTCAATTGGCCAATGTAACCTACAATAGCACCATCAAGCTTGAAACATCCCAGATCTT
GCCTATTGATCCGTTGGATATATCCCAGAATCTAGCTGCGGTGAATAAGAGTCTAAGTGATGCACTAC
AACACTTAGCACAAAGTGACACATACCTTTCTGCAATCACATCAGCTACGACTACAAGTGTATTATCC
ATAATAGCAATCTGTCTTGGATCGTTAGGTTTAATATTAATAATCTTGCTCAGTGTAGTTGTGTGGAAG
TTATTGACCATTGTCGCTGCTAATCGAAATAGAATGGAGAATTTTGTTTATCATAATTCAGCATTCCAC
CACTCACGATCTGATCTCAGTGAGAAAAATCAACCTGCAACTCTTGGAACAAGATAAGACAGTCATCC
ATTAGTAATTTTTAAGAAAAAAACGATAGGACCGAAACTAGTATTGAAAGAACCGTCTCGGTCAATCT
AGGTAATCGAGCTGcTACCGTCTCGGAAAGCTCAAATCATGCTGCCTGATCCGGAAGATCCGGAAAGC
AAAAAAGCTACAAGGAGAACAGGAAACCTAATTATCTGCTTCCTATTCATCTTCTTTCCGTTTGTAAAC
TTCATTGTTCCAACTCTAAGACACTTGCTGTCCTAACACCTGCTATAGGCTATCCACTGCATCATCTCTT
TTTAAGAAAAAAATAGGCCCGGACGGGTTAGCAACAAGCGACTGCCGGTGCCAACAACACAATCCAC
AATCTACAGTCGACGCGGCCGCCACCatgttcgtgttcctggtgctgctgccactggtcagcagccagtg
cgtgaatttcactacccggacccagctgcctcctgcttatacaaacagcttcaccaggggagtgtactaccccg
ataaggtgttccgcagctctgtgctgcacagcacccaggacttgtttcttcccttcttcagcaacgtgac
ctggttccacgccatccacgtgtccggcaccaacggcaccaaaagattcgccaatcctgtgctgcctttcaacgat
ggcgtgtactttgcctctacagagaagtctaacatcatcagaggctggatcttcggcacaaccctggattct
aaaacccagagcctgctgatcgtgaacaatgccaccaatgtagtgatcaaggtttgtgagttccagttct
gcaacgacccctttctgggcgtttactaccacaagaacaacaaaagctggatggaaagcgagtt
cagggtgtactctagcgccaacaactgtaccttcgagtacgtgagccagccttttctgatggacctggaggg
caagcagggcaacttcaaaaacctgcgggagttcgtgtttaagaacatcgacggatacttcaagatt
tactccaagcacaccccaattaacttggttagaggactgccccaaggcttcagcgccctggaacccctggt
ggacctgcctatcggcatcaacatcacaagattccaaaccctgcatagaagctatctgacaccaggcgactctagc
tctggatggacagccggcgccgctgcttattacgtgggctacctgcagcctagaaccttcctcctg
aaatacaacgagaacggaaccatcaccgacgccgtggactgcgccctggatcctctgagcgagacaaagtgcac
cctgaagtccttcaccgtcgaaaagggcatctaccaaacaagcaacttcagagtgcagcctactgagagcatcgtg
cggtttccaaacatcaccaacctgtgtccttttggcgaggtgttcaacgccacccggttcgcttccgtgt
acgcctggaatagaaagcggatcagcaactgcgtggccgactacagcgtgctgtata
atagcgcctcattttctacttttaaatgttacggcgtgtctcctacaaagctgaacgatc
tgtgcttcacaaatgtgtacgccgatagcttcgtgattagaggcgacgaggtgcggcagatcgc
ccctggccagaccggcaacatcgctgactacaactacaagctgcctgacgacttcaccggct
gtgtgatcgcatggaacagcaataacctggactctaaggtgggcggaaactacaattacctgtaca
gactgttcagaaagagcaacctgaagcccttcgagagagatatctccacagaaatctatca
gccggatctacaccttgtaatggcgtgaagggcttcaactgctacttccctctgcaatc
ctacgggctttcagcccacatacggcgtgggctaccagccataccgggtcgtggtgctg
agcttcgagctgctgcacgcccctgccacagtgtgcggccctaagaagagcaccaatctggtaa
agaacaagtgcgtcaacttcaatttcaacggcctgaccgggaccggagtgctgaccgaaagcaa
caagaaattcctgccgttccagcagttcggcagagatatcgccgacaccaccgatgccgtgcg
ggacccccagacacttgaaatcctggacatcaccccttgcagttttggcggcgtgtccgtgat
cacacctggaacaaacaccagcaaccaggtggccgtgctgtaccagggcgtcaactgcaccgaggtccccgt
ggccatccacgccgatcagctgacacctacatggcgggtgtactctaccggcagcaacgtgttcc
aaaccagagccggctgcctgatcggcgctgagcacgtgaacaacagctacgagtgcgacattcctatcggt
gctggcatctgcgcctcttaccagacacagacaaacagccccagaagagcgagaagcgtggctagtcagagca
tcatcgcctacaccatgagcctgggcgtggagaacagcgtggcctactctaacaatagcatcgccatcccaacca
acttcacaatctctgtgaccacagagatcctgcccgtgtccatgaccaagacatctgtggactgcacaatgtata
tctgtggcgattctaccgagtgcagcaacctgctgctccagtacggcagcttttgtacccagctgaatagagcc
ctgacaggcatcgccgtggagcaggataagaacacacaggaggtgttcgcccaggtga
agcaaatctacaagaccccccctatcaaggactttggcggcttcaatttttc
ccagatcctgcctgatccatccaagccttctaagcggagcttta
tcgaggacctgctgttcaacaaggtgaccctggccgatgccggcttcatc
aagcagtatggcgattgcctgggcgacatcgcagccagggacctgatctgcgc
ccagaagtttaatggcctgaccgtgctgccacccctgctgacagatgagatgatcg
cacagtacacaagcgccctgctggccggcaccatcacatccggatggacctt
cggcgcaggagccgccctccagatcccctttgccatgcagatggcctataggttcaa
cggcatcggcgtgacccagaatgtgctgtacgagaaccagaagctgatcgccaa
tcagtttaactccgccatcggcaagatccaggacagcctgtcctctacagccagc
gccctgggcaagctccaggatgtggtgaatcagaacgcccaggccctgaataccctgg
tgaagcagctgagcagcaacttcggcgccatctctagcgtgctgaatgac
atcctgagccggctggacaaggtggaggcagaggtgcagatcgaccggctgatcacc
ggccggctccagagcctccagacctatgtgacacagcagctgatcaggg
ccgccgagatcagggccagcgccaatctggcagcaaccaagatgtccgagtgcgtgct
gggccagtctaagagagtggacttttgtggcaagggctatcacctgatgt
ccttccctcagtctgccccacacggcgtggtgtttctgcacgtgacctacgtgccc
gcccaggagaagaacttcaccacagcccctgccatctgccacgatggcaaggc
ccactttccaagggagggcgtgttcgtgtccaacggcacccactggtttgtgacacag
cgcaatttctacgagccccagatcatcaccacagacaacaccttcgtgagcg
gcaactgtgacgtggtcatcggcatcgtgaacaataccgtgtatgatccactccagc
ccgagctggacagctttaaggaggagctggataagtatttcaagaatcacacct
cccctgacgtggatctgggcgacatcagcggcatcaatgcctccgtggtgaacatc
cagaaggagatcgaccgcctgaacgaggtggctaagaatctgaacgagagc
ctgatcgacctccaggagctgggcaagtatgagcagtacatcaagtggccctggta
catctggctgggcttcatcgccggcctgatcgccatcgtgatggtgaccatcat
gctgtgctgtatgacatcctgctgttcttgcctgaagggctgctgtagctgt
ggctcctgctgtAAGTTATTGACCATTGTCGTTGCTAATCGAA
ATAGAATGGAGAATTTTGTTTATCATAAATGAACGCGTGCTAGCCTCCTGCCATACTTCCTACTCACAT
CATATCTATTTTAAAGAAAAAATAGGCCCGAACACTAATCGTGCCGGCAGTGCCACTGCACACACAAC
ACTACACATACAATACACTACAATGGTTGCAGAAGATGCCCCTGTTAGGGGCACTTGCCGAGTATTAT
TTCGAACAACAACTTTAATTTTTCTATGCACACTATTAGCATTAAGCATCTCTATCCTTTATGAGAGTTT
AATAACCCAAAAGCAAATCATGAGCCAAGCAGGCTCAACTGGATCTAATTCTAGATTAGGAAGTATCA
CTGATCTTCTTAATAATATTCTCTCTGTCGCAAATCAGATTATATATAACTCTGCAGTCGCTCTACCTCT
ACAATTGGACACTCTTGAATCAACACTCCTTACAGCCATTAAGTCTCTTCAAACAAGTGACAAGCTAG
AACAGAACTGCTCGTGGGGTGCTGCACTGATTAATGATAATAGATACATTAATGGCATCAATCAGTTC
TATTTCTCAATTGCTGAGGGTCGCAAGCTGACACTTGGCCCACTTCTTAATATACCTAGTTTCATTCCA
ACTGCCACGACACCAGAGGGCTGCACCAGGATCCCATCATTCTCGCTCACCAAGACACACTGGTGTTA
TACACACAATGTTATCCTGAATGGATGCCAGGATCATGTATCCTCAAATCAATTTGTTTCCATGGGAAT
CATTGAACCCACTTCTGCCGGGTTTCCATCCTTTCGAACCCTAAAGACTCTATATCTCAGCGATGGGGT
CAATCGTAAGAGCTGCTCTATCAGTACAGTTCCGGGGGGTTGTATGATGTACTGTTTCGTCTCTACTCA
ACCAGAGAGGGATGACTACTTTTCTACCGCTCCTCCAGAACAACGAATTATTATAATGTACTATAATG
ATACAATCGTGGAGCGCATAATTAATCCACCCGGGGTACTAGATGTATGGGCAACATTGAACCCAGGA
ACAGGAAGCGGGGTATATTATTTAGGTTGGGTACTCTTTCCAATATATGGCGGCGTGATTAAAGATAC
GAGTTTATGGAATAATCAAGCAAATAAATACTTTATCCCCCAGATGGTTGCTGCTCTCTGCTCACAAAA
CCAGGCAACTCAAGTCCAAAATGCTAAGTCATCATACTATAGCAGCTGGTTTGGCAATCGAATGATTC
AGTCTGGGATCCTGGCATGTCCTCTTCAACAGGATCTAACCAATGAGTGTTTAGTTCTGCCCTTTTCTA
ATGATCAGGTGCTTATGGGTGCTGAAGGGAGATTATACATGTATGGTGACTCGGTGTATTACTACCAA
AGAAGCAATAGTTGGTGGCCTATGACCATGCTGTATAAGGTAACCATAACATTCACTAATGGTCAGCC
ATCTGCTATATCAGCTCAGAATGTGCCCACACAGCAGGTCCCTAGACCTGGGACAGGAAGCTGCTCTG
CAACAAATAGATGTCCCGGTTTTTGCTTGAAAGGAGTGTATGCTGATGCCTGGTTACTGACCAACCCTT
CGTCTACCAGTACATTTGGATCAGAAGCAACCTTCACTGGTTCTTATCTCAACGCAGCAACTCAGCGTA
TCAATCCGACGATGTATATCGCGAACAACACACAGATCATAAGCTCACAGCAATTTGGATCAAGCGGT
CAAGAAGCAGCATATGGCCACACAACTTGTTTTAGGGACACAGGCTCTGTTATGGTATACTGTATCTA
TATTATTGAATTGTCCTCATCTCTCTTAGGACAATTTCAGATTGTCCCATTTATCCGTCAGGTGACACTA
TCCTAAAGGCAGAAGCCTCCAGGTCTGACCCAGCCAATCAAAGCATTATACCAGACCATGGAATGCAT
ACCAAACATTATTGACACTAATGACACACAAAATTGGTTTTAAGAAAAACCAAGAGAACAATAGGCC
AGAATGGCTGGGTCTCGGGAGATATTACTCCCTGAAGTCCATCTCAATTCACCAATTGTAAAGCATAA
GCTATACTATTACATTCTACTTGGAAACCTCCCAAATGAGATCGACATTGACGATTTAGGTCCATTACA
TAATCAAAATTGGAATCAAATAGCACATGAAGAGTCTAACTTAGCCCAACGCTTGGTAAATGTAAGAA
ATTTTCTAATTACCCACATCCCTGATCTTAGAAAGGGCCATTGGCAAGAGTATGTCAATGTAATACTGT
GGCCGCGAATTCTTCCCTTGATCCCGGATTTTAAAATCAATGACCAATTGCCTCTACTCAAAAATTGGG
ACAAGTTAGTTAAAGAATCATGTTCAGTAATCAATGCGGGTACTTCCCAGTGCATTCAGAATCTCAGC
TATGGACTGACAGGTCGTGGGAACCTCTTTACACGATCACGTGAACTCTCTGGTGACCGCAGGGATAT
TGATCTTAAGACGGTTGTGGCAGCATGGCATGACTCAGACTGGAAAAGAATAAGTGATTTTTGGATTA
TGATCAAATTCCAGATGAGACAATTAATTGTTAGGCAAACAGATCATAATGATCCTGATTTAATCACG
TATATCGAAAATAGAGAAGGCATAATCATCATAACCCCTGAACTGGTAGCATTATTTAACACTGAGAA
TCATACACTAACATACATGACCTTTGAAATTGTACTGATGGTTTCAGATATGTACGAAGGTCGTCACAA
CATTTTATCACTATGCACAGTTAGCACTTACCTGAATCCTCTGAAGAAAAGAATAACATATTTATTGAG
CCTTGTAGATAACTTAGCTTTTCAGATAGGTGATGCTGTATATAACATAATTGCTTTGCTAGAATCCTT
TGTATATGCACAGTTGCAAATGTCAGATCCCATCCCAGAACTCAGAGGACAATTCCATGCATTCGTAT
GTTCTGAGATTCTTGATGCACTAAGGGGAACTAATAGTTTCACCCAGGATGAATCAAGAACTGTGACA
ACCAATTTGATATCCCCATTCCAAGATCTGACCCCAGATCTTACGGCTGAATTGCTCTGTATAATGAGG
CTTTGGGGACACCCCATGCTCACCGCCAGTCAAGCTGCGGGAAAGGTACGCGAGTCCATGTGTGCTGG
AAAAGTATTAGACTTTCCCACCATTATGAAAACACTAGCCTTTTTCCATACTATTCTGATCAATGGATA
CAGGAGGAAGCATCATGGAGTATGGCCACCCTTAAACTTACCGGGTAATGCTTCAAAGGGTCTCACGG
AACTTATGAATGACAATACTGAGATAAGCTATGAATTCACACTTAAGCATTGGAAGGAAATCTCTCTT
ATAAAATTCAAGAAATGTTTTGATGCAGACGCAGGTGAGGAACTCAGTATATTTATGAAAGATAAAGC
AATTAGTGCCCCAAAACAAGATTGGATGAGTGTGTTTAGAAGAAGCCTAATCAAACAGCGCCATCAGC
ATCATCAGGTCCCCCTACCAAATCCATTCAATCGACGGCTATTGCTAAACTTTCTCGGAGATGACAAAT
TCGACCCGAATGTGGAGCTACAGTATGTAACATCAGGTGAGTATCTACATGATGACACGTTTTGTGCA
TCATATTCACTAAAAGAGAAGGAAATTAAACCTGATGGTCGAATTTTTGCAAAGTTGACTAAGAGAAT
GAGATCATGTCAAGTTATAGCAGAATCTCTTTTAGCGAACCATGCTGGGAAGTTAATGAAAGAGAATG
GTGTTGTGATGAATCAGCTATCATTAACAAAATCACTATTAACAATGAGTCAGATTGGAATAATATCC
GAGAAAGCTAGAAAATCGACTCGAGATAACATAAATCAACCTGGTTTCCAGAATATCCAGAGAAATA
AATCACATCACTCCAAGCAAGTCAATCAGCGAGATCCAAGTGATGACTTTGAATTGGCAGCATCTTTT
TTAACTACTGATCTCAAAAAATATTGTTTACAATGGAGGTACCAGACAATTATCCCATTTGCTCAATCA
TTAAACAGAATGTATGGTTATCCTCATCTCTTTGAGTGGATTCACTTACGGCTAATGCGTAGTACACTT
TACGTGGGGGATCCCTTCAACCCACCAGCAGATACCAGTCAATTTGATCTAGATAAAGTAATTAATGG
AGATATCTTCATTGTATCACCCAGAGGTGGAATTGAAGGGCTATGTCAAAAGGCTTGGACAATGATAT
CTATCTCTGTGATAATTCTATCTGCCACAGAGTCTGGCACACGAGTAATGAGTATGGTGCAGGGAGAT
AATCAAGCAATTGCTGTCACCACACGAGTACCAAGGAGCCTGCCGACTCTTGAGAAAAAGACTATTGC
TTTTAGATCTTGTAATCTATTCTTTGAGAGGTTAAAATGTAATAATTTTGGATTAGGTCACCATTTGAA
AGAACAAGAGACTATCATTAGTTCTCACTTCTTTGTTTATAGCAAGAGAATATTCTATCAGGGGAGGA
TTCTAACGCAAGCCTTAAAAAATGCTAGTAAGCTCTGCTTGACAGCTGATGTCCTAGGAGAATGTACC
CAATCATCATGTTCTAATCTTGCAACTACTGTCATGAGGTTAACTGAGAATGGTGTTGAAAAAGATATC
TGTTTCTACTTGAATATCTATATGACCATCAAACAGCTCTCCTATGATATCATCTTCCCTCAAGTGTCAA
TTCCTGGAGATCAGATCACATTAGAATACATAAATAATCCACACCTGGTATCACGATTGGCTCTTCTGC
CATCCCAGCTAGGAGGTCTAAACTACCTGTCATGCAGTAGGCTGTTCAATCGAAACATAGGCGACCCG
GTGGTTTCCGCAGTTGCAGATCTTAAGAGATTAATTAAATCAGGATGTATGGATTACTGGATCCTTTAT
AACTTATTAGGGAGAAAACCGGGAAACGGCTCATGGGCTACTTTAGCAGCTGACCCGTACTCAATCAA
TATAGAGTATCAATACCCCCCAACTACAGCTCTTAAGAGGCACACCCAACAAGCTCTGATGGAACTCA
GTACGAATCCAATGTTACGTGGCATATTCTCTGACAATGCACAGGCAGAAGAAAATAATCTTGCTAGA
TTTCTCCTGGATAGGGAGGTGATCTTTCCGCGTGTAGCTCACATCATCATTGAGCAAACCAGTGTCGGG
AGGAGAAAACAGATTCAAGGATATTTGGATTCAACTAGATCGATAATGAGTAAATCACTAGAAATTA
AGCCCTTGTCCAATAGGAAGCTTAATGAAATACTGGATTACAACATCAATTACCTAGCTTACAATTTG
GCATTACTCAAGAATGCTATTGAACCTCCGACTTATTTGAAAGCAATGACTCTTGAAACATGTAGCATC
GACATTGCAAGGAGCCTCCGGAAGCTCTCCTGGGCCCCACTCTTGGGTGGGAGAAATCTTGAAGGATT
AGAGACGCCAGATCCCATTGAAATTACTGCAGGAGCATTAATTGTTGGATCGGGCTACTGTGAACAGT
GTGCTGCAGGAGACAATCGATTCACATGGTTTTTCTTGCCATCTGGTATCGAGATAGGAGGGGATCCC
CGTGATAATCCTCCTATCCGTGTACCGTACATTGGCTCCAGGACTGATGAGAGGAGGGTAGCCTCAAT
GGCATACATCAGGGGTGCCTCGAGTAGCCTAAAAGCAGTTCTTAGACTGGCGGGAGTGTACATCTGGG
CATTCGGAGATACTCTGGAGAATTGGATAGATGCACTGGATTTGTCTCACACTAGAGTTAACATCACA
CTTGAACAGCTGCAATCCCTCACCCCACTTCCAACCTCTGCCAATCTAACCCATCGGTTGGATGATGGC
ACAACTACCCTAAAGTTTACTCCTGCGAGCTCTTACACCTTTTCAAGTTTCACTCATATATCAAATGAT
GAGCAATACCTGACAATTAATGACAAAACTGCAGATTCAAATATAATCTACCAACAGTTAATGATCAC
TGGACTCGGAATTTTAGAAACATGGAATAATCCCCCAATCAATAGAACATTCGAAGAATCTACCCTAC
ATTTGCACACTGGTGCATCATGTTGTGTCCGACCTGTGGACTCCTGCATCATCTCAGAAGCATTAACAG
TCAAGCCACATATTACAGTACCGTACAGCAATAAATTTGTATTTGATGAAGACCCGCTATCTGAATAT
GAGACTGCAAAACTGGAATCGTTATCATTTCAAGCCCAATTAGGCAACATTGATGCTGTAGATATGAC
AGGTAAATTAACATTATTGTCCCAATTCACTGCAAGGCAGATTATTAATGCAATCACTGGACTCGATG
AGTCTGTCTCTCTTACTAATGATGCCATTGTTGCATCAGACTATGTCTCCAATTGGATTAGTGAATGCA
TGTATACCAAATTAGATGAATTATTTATGTATTGTGGGTGGGAACTACTATTGGAACTATCCTATCAAA
TGTATTATCTGAGGGTAGTTGGGTGGAGTAATATAGTGGATTATTCTTACATGATCTTGAGAAGAATCC
CGGGTGCAGCATTAAACAATCTGGCATCTACATTAAGTCATCCAAAACTTTTCCGACGAGCTATCAAC
CTAGATATAGTTGCCCCCTTAAATGCTCCTCATTTTGCATCTCTGGACTACATCAAGATGAGTGTGGAT
GCAATACTCTGGGGCTGTAAAAGAGTCATCAATGTGCTCTCCAATGGAGGGGACTTAGAATTAGTTGT
GACATCTGAAGATAGCCTTATTCTCAGTGACCGATCCATGAATCTCATTGCAAGGAAATTAACTTTATT
ATCACTGATTCACCATAATGGTTTGGAACTACCAAAGATTAAGGGGTTCTCTCCTGATGAGAAGTGTTT
CGCTTTGACAGAATTTTTGAGGAAAGTGGTGAACTCAGGGTTGAGTTCAATAGAGAACCTATCAAATT
TTATGTACAATGTGGAGAACCCACGGCTTGCAGCATTCGCCAGCAACAATTACTACCTGACCAGAAAA
TTATTGAATTCAATACGAGATACTGAGTCGGGTCAAGTAGCAGTCACCTCATATTATGAATCATTAGA
ATATATTGATAGTCTTAAGCTAACCCCACATGTGCCTGGTACCTCATGCATTGAGGATGATAGTCTATG
TACAAATGATTACATAATCTGGATCATAGAGTCTAATGCAAACTTGGAGAAGTATCCAATTCCAAATA
GCCCTGAGGATGATTCCAATTTCCATAACTTTAAGTTGAATGCTCCATCGCACCATACCTTACGCCCAT
TAGGGTTGTCATCAACTGCTTGGTATAAGGGTATAAGCTGTTGCAGGTACCTTGAGCGATTAAAGCTA
CCACAAGGTGATCATTTATATATTGCAGAAGGTAGTGGTGCCAGTATGACAATCATAGAATACCTATT
CCCAGGAAGAAAGATATATTACAATTCTTTATTTAGTAGTGGTGACAATCCCCCACAAAGAAATTATG
CACCAATGCCTACTCAGTTCATTGAGAGTGTCCCATACAAGCTCTGGCAAGCACACACAGATCAATAT
CCCGAGATTTTTGAGGACTTCATCCCTCTATGGAACGGAAATGCCGCCATGACTGACATAGGAATGAC
AGCTTGTGTAGAATTCATCATCAATCGAGTCGGCCCAAGGACTTGCAGTTTAGTACATGTAGATTTGG
AATCAAGTGCAAGCTTAAATCAACAATGCCTGTCAAAGCCGATAATTAATGCTATCATCACTGCTACA
ACTGTTTTGTGCCCTCATGGGGTGCTTATTCTGAAATATAGTTGGTTGCCATTTACTAGATTTAGTACTT
TGATCACTTTCTTATGGTGCTACTTTGAGAGAATCACTGTTCTTAGGAGCACATATTCTGGTCCAGCTA
ATCATGAGGTTTATTTAATTTGTATCCTTGCCAACAACTTTGCATTCCAGACTGTCTCGCAGGCAACAG
GAATGGCGATGACTTTAACCGATCAAGGGTTTACTTTGATATCACCTGAAAGAATAAATCAGTATTGG
GATGGTCACTTGAAGCAAGAACGTATCGTAGCAGAAGCAATTGATAAGGTGGTTCTAGGAGAAGATG
CTCTATTCAATTCGAGTGATAATGAATTAATTCTCAAATGTGGAGGGACACCAAATGCACGGAATCTT
ATCGATATCGAGCCAGTCGCAACTTTCATAGAATTTGAACAACTGATCTGCACAATGTTGACAACCCA
CTTGAAGGAAATAATTGATATAACAAGGTCTGGAACCCAGGATTATGAAAGTTTATTACTCACTCCTT
ACAATTTAGGTCTTCTTGGTAAAATCAGTACGATAGTGAGATTATTAACAGAAAGGATTCTAAATCAT
ACTATCAGGAATTGGTTGATCCTCCCACCTTCGCTCCGGATGATCGTGAAGCAGGACTTGGAATTCGG
CATATTCAGGATTACTTCCATCCTCAATTCTGATCGGTTCCTGAAGCTTTCTCCAAATAGGAAATACTT
GATTACACAATTAACTGCAGGCTACATTAGGAAATTGATTGAGGGGGATTGTAATATCGATCTAACCA
GACCTATCCAAAAACAAATCTGGAAAGCATTAGGTTGTGTAGTCTATTGTCACGATCCAGTAGATCAA
AGGGAATCAACAGAGTTTATTGATATAAATATTAATGAAGAAATAGACCGCGGGATCGATGGCGAGG
AAATCTAAATATATCAAGAATCAGAATTAGTTTAAGAAAAAAGAAGAGGATTAATCTTGGTTTTCCCC
TTGGT.

10. CVL58

The nucleic acid sequence for CVL58 is:

(SEQ ID NO: 70)
ACCAGGGGGAAAACGAAGTGGTGACTCAAATCATCGAAGACCCTCGAGATTACATAGGTCCGGAACC
TATGGCCTTCGTGACCGACCTCGAGTCAGAGTAGTTCAATAAGGACCTATCAAGTTTGGGCAATTTTTC
GTCCCCGACACAAAAATGTCATCCGTGCTTAAAGCATATGAGCGATTCACACTCACTCAAGAACTGCA
AGATCAGAGTGAGGAAGGTACAATCCCACCTACAACACTAAAACCGGTAATCAGGGTATTTATACTAA
CCTCTAATAACCCAGAGCTAAGATCCCGGCTTCTTCTATTCTGCCTACGGATTGTTCTCAGTAATGGTG
CAAGGGATTCCCATCGCTTTGGAGCATTACTTACAATGTTTTCGCTACCATCAGCCACAATGCTCAATC
ATGTCAAATTAGCTGACCAGTCACCAGAAGCTGATATCGAAAGGGTAGAGATCGATGGCTTTGAGGA
GGGATCATTCCGCTTAATCCCCAATGCTCGTTCAGGTATGAGCCGTGGAGAGATCAATGCCTATGCTG
CACTTGCAGAAGATCTACCTGACACACTAAACCATGCAACACCTTTTGTTGATTCCGAAGTCGAGGGA
ACTGCATGGGATGAGATTGAGACTTTCTTAGATATGTGTTACAGTGTCCTAATGCAGGCATGGATAGT
GACTTGCAAGTGCATGACTGCGCCAGACCAACCTGCTGCTTCTATTGAGAAACGCCTGCAAAAATATC
GTCAGCAAGGCAGGATCAACCCGAGATATCTCCTGCAACCGGAGGCTCGAAGAATAATCCAGAATGT
AATCCGAAAGGGAATGGTGGTCAGACATTTCCTCACCTTTGAACTGCAGCTTGCCCGAGCACAAAGCC
TTGTATCAAATAGGTATTATGCTATGGTAGGGGATGTTGGAAAGTATATAGAGAATTGTGGAATGGGA
GGCTTCTTTTTGACACTAAAATATGCATTAGGAACCAGATGGCCCACACTTGCTTTAGCTGCATTTTCA
GGAGAGCTAACAAAGCTAAAGTCCCTCATGGCATTATACCAGACCCTTGGTGAGCAGGCCCGATATTT
GGCCCTATTGGAGTCACCACATTTGATGGATTTTGCTGCAGCAAACTACCCACTGCTATATAGCTATGC
TATGGGAATAGGCTATGTGTTAGATGTCAACATGAGGAACTACGCTTTCTCCAGATCATACATGAATA
AGACATATTTCCAATTGGGAATGGAAACTGCAAGAAAACAACAGGGTGCAGTTGACATGAGGATGGC
AGAAGATCTCGGTCTAACTCAAGCCGAACGCACCGAGATGGCAAATACACTTGCCAAATTGACCACA
GCAAATCGAGGGGCAGACACCAGGGGAGGAGTCAACCCGTTCTCATCTATCACTGGGACAACTCAGG
TGCCCGCTGCAGCAACAGGTGACACATTCGAGAGTTACATGGCAGCGGATCGACTGAGGCAGAGATA
TGCTGATGCAGGCACCCACGATGATGAGATGCCACCATTGGAAGAGGAGGAAGAGGACGACACATCT
GCAGGTCCACGCACTGGACTAACTCTTGAACAAGTGGCCTTGGACATCCAGAACGCAGCAGTTGGAGC
TCCCATCCATACAGATGACCTGAATGCCGCACTGGGTGATCTTGACATCTAGACAATTCAGATCCCAA
TCTTAAATCGACACACCTAATTGACCAGTTAGATGGAACTACAGTGGATTCCATAAGGTTCCTGCCTAC
CATCGGCTTTTAAGAAAAAAATAGGCCCGGACGGGTTAGCAACAAGCGACTGCCGATGCCAATAACA
CAATCCACAATCTACAATGGATCCCACTGATCTGAGCTTCTCCCCAGATGAGATCAATAAGCTCATAG
AGACAGGCCTGAATACTGTGGAGTATTTTACTTCCCAACAAGTCACAGGAACATCCTCTCTTGGAAAG
AATACAATACCACCAGGGGTCACAGGACTACTAACCAATGCTGCAGAGGCAAAGATCCAAGAGTCAA
CCAACCATCAGAAGGGTTCAGTTGGTGGGGGCGCAAAACCAAAGAAACCGCGACCAAAAATTGCCAT
TGTGCCAGCAGATGACAAAACGGTGCCCGGAAAGCCGATCCCAAACCCTCTATTAGGTCTGGACTCCA
CCCCGAGCACCCAAACTGTGCTTGATCTAAGTGGGAAAACATTACCATCAGGATCCTATAAGGGGGTT
AAACTTGCGAAATTTGGAAAAGAAAATCTGATGACACGGTTCATCGAGGAACCCAGAGAGAATCCTA
TCGCAACCAATTCCCCCATCGATTTTAAGAGGGGCAGGGATACCGGCGGGTTCCATAGAAGGGAGTAC
TCAATCGGATGGGTGGGAGATGAAGTCAAGGTCACTGAGTGGTGCAATCCATCCTGTTCTCCAATCAC
CGCTGCAGCAAGGCGATTTGAATGCACTTGTCACCAGTGTCCAGTCACTTGCTCTGAATGTGAACGAG
ATACTTAATACAGTGAGAAATTTGGACTCTCGGATGAATCAACTGGAGACAAAAGTAGATCGCATTCT
CTCATCTCAGTCTCTAATCCAGACCATCAAGAATGACATAGTTGGACTTAAAGCAGGGATGGCTACTT
TAGAAGGAATGATTACAACTGTGAAAATCATGGACCCGGGAGTTCCCAGTAATGTTACTGTGGAAGAT
GTACGCAAGAAACTAAGTAACCATGCTGTTGTTGTGCCAGAATCATTCAATGATAGTTTCTTGACTCAA
TCTGAAGATGTAATTTCACTTGATGAGTTGGCTCGACCAACTGCAACAAGTGTTAAGAAGATTGTCAG
GAAGGTTCCTCCTCAGAAGGATCTGACTGGATTGAAGATCACACTAGAGCAATTGGCAAAGGATTGCA
TCAGCAAACCGAAGATGAGGGAAGAGTATCTCCTCAAAATCAACCAGGCTTCCAGTGAGGCTCAGCT
AATTGACCTCAAGAAAGCAATCATCCGCAGTGCAATTTGATCAAGAAACACCCAATTACACTACACTG
GTATGACACTGTACTAACCCTGAGGGTTTTAGAAAAAACGATTAACGATAAATAAGCCCGAACACTAC
ACACCACCCGAGGCAGCCATGCCATCCATCAGCATCCCCGCAGACCCCACCAATCCACGTCAATCAAT
AAAAGCGTTCCCAATTGTGATCAACCGTGATGGGGGTGAGAAAGGTCGCTTGGTTAAACAACTACGCA
CAACCTACTTGAATGACCTAGATACTCATGAGCCACTGGTGACATTCGTAAATACTTATGGATTCATCT
ACGAACAGGATCGGGGAAATACCATTGTCGGAGAGGATCAACATGGGAAGAAAAGAGAGGCTGTGA
CTGCTGCAATGGTTACCCTTGGATGTGGGCCTAATCTACCATCATTAGGGAATGTCCTGGGACAACTG
AGTGAATTCCAGGTCATTGTTAGGAAGACATCCAGCAAAGCGGAAGAGATGGTCTTTGAAATTGTTAA
GTATCCGAGAATATTTCGGGGTCATACATTAATCCAGAAAGGACTAGTCTGTGTCTCCGCAGAAAAAT
TTGTTAAGTCACCAGGGAAAGTACAATCTGGAATGGACTATCTCTTCATTCCGACATTTCTGTCAGTGA
CTTACTGTCCAGCTGCAATCAAATTTCAGGTACCTGGCCCCATGTTGAAAATGAGATCAAGATACACT
CAGAGCTTACAACTTGAACTAATGATAAGAATCCTGTGTAAGCCCGATTCGCCACTTATGAAGGTCCA
TATCCCTGACAAGGAAGGAAGAGGATGTCTTGTATCAGTATGGTTGCATGTATGCAATATCTTCAAAT
CAGGAAACAAGAATGGCAGTGAGTGGCAGGAATACTGGATGAGAAAGTGTGCTAACATGCAACTTGA
AGTGTCGATTGCAGATATGTGGGGACCAACTATCATAATTCATGCCAGAGGTCACATTCCCAAAAGTG
CTAAGTTGTTTTTTGGAAAGGGTGGATGGAGCTGCCATCCACTTCACGAAGTTGTTCCAAGTGTCACTA
AAACACTATGGTCCGTGGGCTGTGAGATTACAAAGGCGAAGGCAATAATACAAGAGAGTAGCATCTC
TCTTCTCGTGGAGACTACTGACATCATAAGTCCAAAAGTCAAAATTTCATCTAAGCATCGCCGCTTTGG
GAAATCAAATTGGGGTCTGTTCAAGAAAACCAAATCACTGCCCAACCTGACGGAGCTGGAATGACTG
ACCCCCAATCGAGACTACACCACCTCAAACTATAGGTGGGTGGTACCTCAGTGATTAATCTCGTAAGC
ACTGATCGTAGGCTACAACACACTAATATTATCCAGATTAGAGAGCTTAATTAGCTCTGTATTAATAAT
AACACTACTATTCCAATAACTGGAATCACCAGCTTGATTTATCTCCAAAATGATTCAAAGAAAACAAA
TCATATTAAGACTATCCTAAGCACGAACCCATATCGTCCTTCAAATCATGGGTACTATAATTCAATTTC
TGGTGGTCTCCTGTCTATTGGCAGGAGCAGGCAGTCTTGATCCAGCAGCCCTCATGCAAATCGGTGTC
ATTCCAACAAATGTCCGGCAACTTATGTATTATACTGAGGCCTCATCAGCATTCATTGTTGTGAAGTTA
ATGCCTACAATTGACTCGCCGATTAGTGGATGTAATATAACATCAATTTCAAGCTATAATGCAACAGT
GACAAAACTCCTACAGCCGATCGGTGAGAATTTGGAGACGATTAGGAACCAGTTGATTCCAACTCGGA
GGAGACGCCGGTTTGCAGGGGTGGTGATTGGATTAGCTGCATTAGGAGTAGCTACTGCCGCACAGGTC
ACTGCCGCAGTAGCACTAGTAAAGGCAAATGAAAATGCTGCGGCTATACTCAATCTCAAAAATGCAAT
CCAAAAAACAAATGCAGCAGTTGCAGATGTGGTCCAGGCCACACAATCACTAGGAACaGCAGTTCAAG
CAGTTCAAGATCACATAAACAGTGTGGTAAGTCCAGCAATTACAGCAGCCAATTGTAAAGCCCAAGAT
GCTATCATTGGCTCAATCCTCAATCTCTATTTGACCGAGTTGACAACTATCTTCCACAATCAAATTACA
AACCCTGCATTGAGTCCTATTACAATTCAAGCTTTAAGGATCCTACTAGGGAGTACCTTGCCGACTGTG
GTCGAAAAATCTTTCAATACCCAGATAAGTGCGGCTGAGCTTCTCTCATCAGGGTTATTGACAGGCCA
GATTGTGGGATTAGATTTGACCTATATGCAGATGGTCATAAAAATTGAGCTGCCAACTTTAACTGTAC
AACCTGCAACCCAGATCATAGATCTGGCCACCATTTCTGCATTCATTAACAATCAAGAAGTCATGGCC
CAATTACCAACACGTGTTATTGTGACTGGCAGCTTGATCCAAGCCTATCCCGCATCGCAATGCACTATT
ACACCCAACACTGTGTACTGTAGGTATAATGATGCCCAAGTACTCTCAGATGATACGATGGCTTGCCT
CCAAGGTAACTTGACAAGATGCACCTTCTCTCCAGTGGTTGGGAGCTTTCTCACTCGATTCGTGCTGTT
CGATGGAATAGTTTATGCAAATTGCAGGTCGATGTTGTGCAAGTGCATGCAGCCTGCTGCTGTGATCCT
ACAGCCGAGTTCATCCCCTGTAACTGTCATTGACATGTACAAATGTGTGAGTCTGCAGCTTGACAATCT
CAGATTCACCATCACTCAATTGGCCAATGTAACCTACAATAGCACCATCAAGCTTGAAACATCCCAGA
TCTTGCCTATTGATCCGTTGGATATATCCCAGAATCTAGCTGCGGTGAATAAGAGTCTAAGTGATGCAC
TACAACACTTAGCACAAAGTGACACATACCTTTCTGCAATCACATCAGCTACGACTACAAGTGTATTA
TCCATAATAGCAATCTGTCTTGGATCGTTAGGTTTAATATTAATAATCTTGCTCAGTGTAGTTGTGTGG
AAGTTATTGACCATTGTCGCTGCTAATCGAAATAGAATGGAGAATTTTGTTTATCATAATTCAGCATTC
CACCACTCACGATCTGATCTCAGTGAGAAAAATCAACCTGCAACTCTTGGAACAAGATAAGACAGTCA
TCCATTAGTAATTTTTAAGAAAAAAACGATAGGACCGAAACTAGTATTGAAAGAACCGTCTCGGTCAA
TCTAGGTAATCGAGCTGcTACCGTCTCGGAAAGCTCAAATCgcgcgctaacgtacgCACCTGCTATAGGCTATC
CACTGCATCATCTCTTTTTAAGAAAAAAATAGGCCCGGACGGGTTAGCAACAAGCGACTGCCGGTGCC
AACAACACAATCCACAATCTACAGTCGACGCGGCCGCCACCatgttcgtgttcctggtgctgctgccact
ggtcagcagccagtgcgtgaatttcactacccggacccagctgcctcctgcttatacaaacagcttcaccaggggag
tgtactaccccgataaggtgttccgcagctctgtgctgcacagcacccaggacttgtttcttcccttcttcagcaacgtg
acctggttccacgccatccacgtgtccggcaccaacggcaccaaaagattcgccaatcctgtgctgcctttcaacgatggc
gtgtactttgcctctacagagaagtctaacatcatcagaggctggatcttcggcacaaccctggattctaaaa
cccagagcctgctgatcgtgaacaatgccaccaatgtagtgatcaaggtttgtgagttccagttctgcaacgacccct
ttctgggcgtttactaccacaagaacaacaaaagctggatggaaagcgagttcagggtgtactctagcgcca
acaactgtaccttcgagtacgtgagccagccttttctgatggacctggagggcaagcagggcaacttcaaaaacct
gcgggagttcgtgtttaagaacatcgacggatacttcaagatttactccaagcacaccccaattaacttggttagaggact
gccccaaggcttcagcgccctggaacccctggtggacctgcctatcggcatcaacatcacaagattccaaaccctgcat
agaagctatctgacaccaggcgactctagctctggatggacagccggcgccgctgcttattacgtgggctacctgc
agcctagaaccttcctcctgaaatacaacgagaacggaaccatcaccgacgccgtggactgcgccctggatcctctgagcg
agacaaagtgcaccctgaagtccttcaccgtcgaaaagggcatctaccaaacaagcaacttcagagtgcagcctactg
agagcatcgtgcggtttccaaacatcaccaacctgtgtccttttggcgaggtgttcaacgccacccggttcgcttccgtgt
acgcctggaatagaaagcggatcagcaactgcgtggccgactacagcgtgctgtataatagcgcctcattttccgatctg
tgctttacttttaaatgttacggcgtgtctcctacaaagctgaacacaaatgtgtacgccgatagcttcgtgattag
aggcgacgaggtgcggcagatcgcccctggccagaccggcaacatcgctgactacaactacaagctgcctgacgacttc
accggctgtgtgatcgcatggaacagcaataacctggactctaaggtgggcggaaactacaattacctgtacagac
tgttcagaaagagcaacctgaagcccttcgagagagatatctccacagaaatctatcaggccggatctacaccttgtaatgg
cgtgaagggcttcaactgctacttccctctgcaatcctacggctttcagcccacatacggcgtgggctaccagccataccg
ggtcgtggtgctgagcttcgagctgctgcacgcccctgccacagtgtgcggccctaagaagagcaccaatctggta
aagaacaagtgcgtcaacttcaatttcaacggcctgaccgggaccggagtgctgaccgaaagcaacaagaaattcctg
ccgttccagcagttcggcagagatatcgccgacaccaccgatgccgtgcgggacccccagacacttgaaatcctggacatc
accccttgcagttttggcggcgtgtccgtgatcacacctggaacaaacaccagcaaccaggtggccgtgctgtaccaggg
cgtcaactgcaccgaggtccccgtggccatccacgccgatcagctgacacctacatggcgggtgtactctaccgg
cagcaacgtgttccaaaccagagccggctgcctgatcggcgctgagcacgtgaacaacagctacgagtgcgacattcct
atcggtgctggcatctgcgcctcttaccagacacagacaaacagccccagaagagcgagaagcgtggctagtcagagcatc
atcgcctacaccatgagcctgggcgtggagaacagcgtggcctactctaacaatagcatcgccatcccaaccaacttcaca
atctctgtgaccacagagatcctgcccgtgtccatgaccaagacatctgtggactgcacaatgtatatctgtgg
cgattctaccgagtgcagcaacctgctgctccagtacggcagcttttgtacccagctgaatagagccctgacaggcatcg
ccgtggagcaggataagaacacacaggaggtgttcgcccaggtgaagcaaatctacaagaccccccctatcaaggactt
tggcggcttcaatttttcccagatcctgcctgatccatccaagccttctaagcggagctttatcgaggacctg
ctgttcaacaaggtgaccctggccgatgccggcttcatcaagcagtatggcgattgcctgggcgacatcgcagccagg
gacctgatctgcgcccagaagtttaatggcctgaccgtgctgccacccctgctgacagatgagatgatcgcacagtac
acaagcgccctgctggccggcaccatcacatccggatggaccttcggcgcaggagccgccctccagatcccctt
tgccatgcagatggcctataggttcaacggcatcggcgtgacccagaatgtgctgtacgagaaccagaagctgat
cgccaatcagtttaactccgccatcggcaagatccaggacagcctgtcctctacagccagcgccctgggcaagctcca
ggatgtggtgaatcagaacgcccaggccctgaataccctggtgaagcagctgagcagcaacttcggcgccatctctagcgt
gctgaatgacatcctgagccggctggacaaggtggaggcagaggtgcagatcgaccggctgatcaccggccggctc
cagagcctccagacctatgtgacacagcagctgatcagggccgccgagatcagggccagcgccaatctggcagca
accaagatgtccgagtgcgtgctgggccagtctaagagagtggacttttgtggcaagggctatcacctgatgtccttccc
tcagtctgccccacacggcgtggtgtttctgcacgtgacctacgtgcccgcccaggagaagaacttcaccacagc
ccctgccatctgccacgatggcaaggcccactttccaagggagggcgtgttcgtgtccaacggcacccactggtttg
tgacacagcgcaatttctacgagccccagatcatcaccacagacaacaccttcgtgagcggcaactgtgacgtggtcat
cggcatcgtgaacaataccgtgtatgatccactccagcccgagctggacagctttaaggaggagctggataagtatttca
agaatcacacctcccctgacgtggatctgggcgacatcagcggcatcaatgcctccgtggtgaacatccagaag
gagatcgaccgcctgaacgaggtggctaagaatctgaacgagagcctgatcgacctccaggagctgggcaagtatgagcagta
catcaagtggccctggtacatctggctgggcttcatcgccggcctgatcgccatcgtgatggtgaccatcatgctgt
gctgtatgacatcctgctgttcttgcctgaagggctgctgtagctgtggctcctgctgtAAGTTATTGACCA
TTGTCGTTGCTAATCGAAATAGAATGGAGAATTTTGTTTATCATAAATGAACGCGTGCTAGCCTCCTGC
CATACTTCCTACTCACATCATATCTATTTTAAAGAAAAAATAGGCCCGAACACTAATCGTGCCGGCAG
TGCCACTGCACACACAACACTACACATACAATACACTACAATGGTTGCAGAAGATGCCCCTGTTAGGG
GCACTTGCCGAGTATTATTTCGAACAACAACTTTAATTTTTCTATGCACACTATTAGCATTAAGCATCT
CTATCCTTTATGAGAGTTTAATAACCCAAAAGCAAATCATGAGCCAAGCAGGCTCAACTGGATCTAAT
TCTAGATTAGGAAGTATCACTGATCTTCTTAATAATATTCTCTCTGTCGCAAATCAGATTATATATAAC
TCTGCAGTCGCTCTACCTCTACAATTGGACACTCTTGAATCAACACTCCTTACAGCCATTAAGTCTCTT
CAAACAAGTGACAAGCTAGAACAGAACTGCTCGTGGGGTGCTGCACTGATTAATGATAATAGATACAT
TAATGGCATCAATCAGTTCTATTTCTCAATTGCTGAGGGTCGCAAGCTGACACTTGGCCCACTTCTTAA
TATACCTAGTTTCATTCCAACTGCCACGACACCAGAGGGCTGCACCAGGATCCCATCATTCTCGCTCAC
CAAGACACACTGGTGTTATACACACAATGTTATCCTGAATGGATGCCAGGATCATGTATCCTCAAATC
AATTTGTTTCCATGGGAATCATTGAACCCACTTCTGCCGGGTTTCCATCCTTTCGAACCCTAAAGACTC
TATATCTCAGCGATGGGGTCAATCGTAAGAGCTGCTCTATCAGTACAGTTCCGGGGGGTTGTATGATG
TACTGTTTCGTCTCTACTCAACCAGAGAGGGATGACTACTTTTCTACCGCTCCTCCAGAACAACGAATT
ATTATAATGTACTATAATGATACAATCGTGGAGCGCATAATTAATCCACCCGGGGTACTAGATGTATG
GGCAACATTGAACCCAGGAACAGGAAGCGGGGTATATTATTTAGGTTGGGTACTCTTTCCAATATATG
GCGGCGTGATTAAAGATACGAGTTTATGGAATAATCAAGCAAATAAATACTTTATCCCCCAGATGGTT
GCTGCTCTCTGCTCACAAAACCAGGCAACTCAAGTCCAAAATGCTAAGTCATCATACTATAGCAGCTG
GTTTGGCAATCGAATGATTCAGTCTGGGATCCTGGCATGTCCTCTTCAACAGGATCTAACCAATGAGTG
TTTAGTTCTGCCCTTTTCTAATGATCAGGTGCTTATGGGTGCTGAAGGGAGATTATACATGTATGGTGA
CTCGGTGTATTACTACCAAAGAAGCAATAGTTGGTGGCCTATGACCATGCTGTATAAGGTAACCATAA
CATTCACTAATGGTCAGCCATCTGCTATATCAGCTCAGAATGTGCCCACACAGCAGGTCCCTAGACCT
GGGACAGGAAGCTGCTCTGCAACAAATAGATGTCCCGGTTTTTGCTTGAAAGGAGTGTATGCTGATGC
CTGGTTACTGACCAACCCTTCGTCTACCAGTACATTTGGATCAGAAGCAACCTTCACTGGTTCTTATCT
CAACGCAGCAACTCAGCGTATCAATCCGACGATGTATATCGCGAACAACACACAGATCATAAGCTCAC
AGCAATTTGGATCAAGCGGTCAAGAAGCAGCATATGGCCACACAACTTGTTTTAGGGACACAGGCTCT
GTTATGGTATACTGTATCTATATTATTGAATTGTCCTCATCTCTCTTAGGACAATTTCAGATTGTCCCAT
TTATCCGTCAGGTGACACTATCCTAAAGGCAGAAGCCTCCAGGTCTGACCCAGCCAATCAAAGCATTA
TACCAGACCATGGAATGCATACCAAACATTATTGACACTAATGACACACAAAATTGGTTTTAAGAAAA
ACCAAGAGAACAATAGGCCAGAATGGCTGGGTCTCGGGAGATATTACTCCCTGAAGTCCATCTCAATT
CACCAATTGTAAAGCATAAGCTATACTATTACATTCTACTTGGAAACCTCCCAAATGAGATCGACATT
GACGATTTAGGTCCATTACATAATCAAAATTGGAATCAAATAGCACATGAAGAGTCTAACTTAGCCCA
ACGCTTGGTAAATGTAAGAAATTTTCTAATTACCCACATCCCTGATCTTAGAAAGGGCCATTGGCAAG
AGTATGTCAATGTAATACTGTGGCCGCGAATTCTTCCCTTGATCCCGGATTTTAAAATCAATGACCAAT
TGCCTCTACTCAAAAATTGGGACAAGTTAGTTAAAGAATCATGTTCAGTAATCAATGCGGGTACTTCC
CAGTGCATTCAGAATCTCAGCTATGGACTGACAGGTCGTGGGAACCTCTTTACACGATCACGTGAACT
CTCTGGTGACCGCAGGGATATTGATCTTAAGACGGTTGTGGCAGCATGGCATGACTCAGACTGGAAAA
GAATAAGTGATTTTTGGATTATGATCAAATTCCAGATGAGACAATTAATTGTTAGGCAAACAGATCAT
AATGATCCTGATTTAATCACGTATATCGAAAATAGAGAAGGCATAATCATCATAACCCCTGAACTGGT
AGCATTATTTAACACTGAGAATCATACACTAACATACATGACCTTTGAAATTGTACTGATGGTTTCAGA
TATGTACGAAGGTCGTCACAACATTTTATCACTATGCACAGTTAGCACTTACCTGAATCCTCTGAAGAA
AAGAATAACATATTTATTGAGCCTTGTAGATAACTTAGCTTTTCAGATAGGTGATGCTGTATATAACAT
AATTGCTTTGCTAGAATCCTTTGTATATGCACAGTTGCAAATGTCAGATCCCATCCCAGAACTCAGAGG
ACAATTCCATGCATTCGTATGTTCTGAGATTCTTGATGCACTAAGGGGAACTAATAGTTTCACCCAGGA
TGAATCAAGAACTGTGACAACCAATTTGATATCCCCATTCCAAGATCTGACCCCAGATCTTACGGCTG
AATTGCTCTGTATAATGAGGCTTTGGGGACACCCCATGCTCACCGCCAGTCAAGCTGCGGGAAAGGTA
CGCGAGTCCATGTGTGCTGGAAAAGTATTAGACTTTCCCACCATTATGAAAACACTAGCCTTTTTCCAT
ACTATTCTGATCAATGGATACAGGAGGAAGCATCATGGAGTATGGCCACCCTTAAACTTACCGGGTAA
TGCTTCAAAGGGTCTCACGGAACTTATGAATGACAATACTGAGATAAGCTATGAATTCACACTTAAGC
ATTGGAAGGAAATCTCTCTTATAAAATTCAAGAAATGTTTTGATGCAGACGCAGGTGAGGAACTCAGT
ATATTTATGAAAGATAAAGCAATTAGTGCCCCAAAACAAGATTGGATGAGTGTGTTTAGAAGAAGCCT
AATCAAACAGCGCCATCAGCATCATCAGGTCCCCCTACCAAATCCATTCAATCGACGGCTATTGCTAA
ACTTTCTCGGAGATGACAAATTCGACCCGAATGTGGAGCTACAGTATGTAACATCAGGTGAGTATCTA
CATGATGACACGTTTTGTGCATCATATTCACTAAAAGAGAAGGAAATTAAACCTGATGGTCGAATTTT
TGCAAAGTTGACTAAGAGAATGAGATCATGTCAAGTTATAGCAGAATCTCTTTTAGCGAACCATGCTG
GGAAGTTAATGAAAGAGAATGGTGTTGTGATGAATCAGCTATCATTAACAAAATCACTATTAACAATG
AGTCAGATTGGAATAATATCCGAGAAAGCTAGAAAATCGACTCGAGATAACATAAATCAACCTGGTTT
CCAGAATATCCAGAGAAATAAATCACATCACTCCAAGCAAGTCAATCAGCGAGATCCAAGTGATGAC
TTTGAATTGGCAGCATCTTTTTTAACTACTGATCTCAAAAAATATTGTTTACAATGGAGGTACCAGACA
ATTATCCCATTTGCTCAATCATTAAACAGAATGTATGGTTATCCTCATCTCTTTGAGTGGATTCACTTAC
GGCTAATGCGTAGTACACTTTACGTGGGGGATCCCTTCAACCCACCAGCAGATACCAGTCAATTTGAT
CTAGATAAAGTAATTAATGGAGATATCTTCATTGTATCACCCAGAGGTGGAATTGAAGGGCTATGTCA
AAAGGCTTGGACAATGATATCTATCTCTGTGATAATTCTATCTGCCACAGAGTCTGGCACACGAGTAA
TGAGTATGGTGCAGGGAGATAATCAAGCAATTGCTGTCACCACACGAGTACCAAGGAGCCTGCCGACT
CTTGAGAAAAAGACTATTGCTTTTAGATCTTGTAATCTATTCTTTGAGAGGTTAAAATGTAATAATTTT
GGATTAGGTCACCATTTGAAAGAACAAGAGACTATCATTAGTTCTCACTTCTTTGTTTATAGCAAGAGA
ATATTCTATCAGGGGAGGATTCTAACGCAAGCCTTAAAAAATGCTAGTAAGCTCTGCTTGACAGCTGA
TGTCCTAGGAGAATGTACCCAATCATCATGTTCTAATCTTGCAACTACTGTCATGAGGTTAACTGAGAA
TGGTGTTGAAAAAGATATCTGTTTCTACTTGAATATCTATATGACCATCAAACAGCTCTCCTATGATAT
CATCTTCCCTCAAGTGTCAATTCCTGGAGATCAGATCACATTAGAATACATAAATAATCCACACCTGGT
ATCACGATTGGCTCTTCTGCCATCCCAGCTAGGAGGTCTAAACTACCTGTCATGCAGTAGGCTGTTCAA
TCGAAACATAGGCGACCCGGTGGTTTCCGCAGTTGCAGATCTTAAGAGATTAATTAAATCAGGATGTA
TGGATTACTGGATCCTTTATAACTTATTAGGGAGAAAACCGGGAAACGGCTCATGGGCTACTTTAGCA
GCTGACCCGTACTCAATCAATATAGAGTATCAATACCCCCCAACTACAGCTCTTAAGAGGCACACCCA
ACAAGCTCTGATGGAACTCAGTACGAATCCAATGTTACGTGGCATATTCTCTGACAATGCACAGGCAG
AAGAAAATAATCTTGCTAGATTTCTCCTGGATAGGGAGGTGATCTTTCCGCGTGTAGCTCACATCATCA
TTGAGCAAACCAGTGTCGGGAGGAGAAAACAGATTCAAGGATATTTGGATTCAACTAGATCGATAAT
GAGTAAATCACTAGAAATTAAGCCCTTGTCCAATAGGAAGCTTAATGAAATACTGGATTACAACATCA
ATTACCTAGCTTACAATTTGGCATTACTCAAGAATGCTATTGAACCTCCGACTTATTTGAAAGCAATGA
CTCTTGAAACATGTAGCATCGACATTGCAAGGAGCCTCCGGAAGCTCTCCTGGGCCCCACTCTTGGGT
GGGAGAAATCTTGAAGGATTAGAGACGCCAGATCCCATTGAAATTACTGCAGGAGCATTAATTGTTGG
ATCGGGCTACTGTGAACAGTGTGCTGCAGGAGACAATCGATTCACATGGTTTTTCTTGCCATCTGGTAT
CGAGATAGGAGGGGATCCCCGTGATAATCCTCCTATCCGTGTACCGTACATTGGCTCCAGGACTGATG
AGAGGAGGGTAGCCTCAATGGCATACATCAGGGGTGCCTCGAGTAGCCTAAAAGCAGTTCTTAGACTG
GCGGGAGTGTACATCTGGGCATTCGGAGATACTCTGGAGAATTGGATAGATGCACTGGATTTGTCTCA
CACTAGAGTTAACATCACACTTGAACAGCTGCAATCCCTCACCCCACTTCCAACCTCTGCCAATCTAAC
CCATCGGTTGGATGATGGCACAACTACCCTAAAGTTTACTCCTGCGAGCTCTTACACCTTTTCAAGTTT
CACTCATATATCAAATGATGAGCAATACCTGACAATTAATGACAAAACTGCAGATTCAAATATAATCT
ACCAACAGTTAATGATCACTGGACTCGGAATTTTAGAAACATGGAATAATCCCCCAATCAATAGAACA
TTCGAAGAATCTACCCTACATTTGCACACTGGTGCATCATGTTGTGTCCGACCTGTGGACTCCTGCATC
ATCTCAGAAGCATTAACAGTCAAGCCACATATTACAGTACCGTACAGCAATAAATTTGTATTTGATGA
AGACCCGCTATCTGAATATGAGACTGCAAAACTGGAATCGTTATCATTTCAAGCCCAATTAGGCAACA
TTGATGCTGTAGATATGACAGGTAAATTAACATTATTGTCCCAATTCACTGCAAGGCAGATTATTAATG
CAATCACTGGACTCGATGAGTCTGTCTCTCTTACTAATGATGCCATTGTTGCATCAGACTATGTCTCCA
ATTGGATTAGTGAATGCATGTATACCAAATTAGATGAATTATTTATGTATTGTGGGTGGGAACTACTAT
TGGAACTATCCTATCAAATGTATTATCTGAGGGTAGTTGGGTGGAGTAATATAGTGGATTATTCTTACA
TGATCTTGAGAAGAATCCCGGGTGCAGCATTAAACAATCTGGCATCTACATTAAGTCATCCAAAACTT
TTCCGACGAGCTATCAACCTAGATATAGTTGCCCCCTTAAATGCTCCTCATTTTGCATCTCTGGACTAC
ATCAAGATGAGTGTGGATGCAATACTCTGGGGCTGTAAAAGAGTCATCAATGTGCTCTCCAATGGAGG
GGACTTAGAATTAGTTGTGACATCTGAAGATAGCCTTATTCTCAGTGACCGATCCATGAATCTCATTGC
AAGGAAATTAACTTTATTATCACTGATTCACCATAATGGTTTGGAACTACCAAAGATTAAGGGGTTCTC
TCCTGATGAGAAGTGTTTCGCTTTGACAGAATTTTTGAGGAAAGTGGTGAACTCAGGGTTGAGTTCAA
TAGAGAACCTATCAAATTTTATGTACAATGTGGAGAACCCACGGCTTGCAGCATTCGCCAGCAACAAT
TACTACCTGACCAGAAAATTATTGAATTCAATACGAGATACTGAGTCGGGTCAAGTAGCAGTCACCTC
ATATTATGAATCATTAGAATATATTGATAGTCTTAAGCTAACCCCACATGTGCCTGGTACCTCATGCAT
TGAGGATGATAGTCTATGTACAAATGATTACATAATCTGGATCATAGAGTCTAATGCAAACTTGGAGA
AGTATCCAATTCCAAATAGCCCTGAGGATGATTCCAATTTCCATAACTTTAAGTTGAATGCTCCATCGC
ACCATACCTTACGCCCATTAGGGTTGTCATCAACTGCTTGGTATAAGGGTATAAGCTGTTGCAGGTACC
TTGAGCGATTAAAGCTACCACAAGGTGATCATTTATATATTGCAGAAGGTAGTGGTGCCAGTATGACA
ATCATAGAATACCTATTCCCAGGAAGAAAGATATATTACAATTCTTTATTTAGTAGTGGTGACAATCCC
CCACAAAGAAATTATGCACCAATGCCTACTCAGTTCATTGAGAGTGTCCCATACAAGCTCTGGCAAGC
ACACACAGATCAATATCCCGAGATTTTTGAGGACTTCATCCCTCTATGGAACGGAAATGCCGCCATGA
CTGACATAGGAATGACAGCTTGTGTAGAATTCATCATCAATCGAGTCGGCCCAAGGACTTGCAGTTTA
GTACATGTAGATTTGGAATCAAGTGCAAGCTTAAATCAACAATGCCTGTCAAAGCCGATAATTAATGC
TATCATCACTGCTACAACTGTTTTGTGCCCTCATGGGGTGCTTATTCTGAAATATAGTTGGTTGCCATTT
ACTAGATTTAGTACTTTGATCACTTTCTTATGGTGCTACTTTGAGAGAATCACTGTTCTTAGGAGCACA
TATTCTGGTCCAGCTAATCATGAGGTTTATTTAATTTGTATCCTTGCCAACAACTTTGCATTCCAGACTG
TCTCGCAGGCAACAGGAATGGCGATGACTTTAACCGATCAAGGGTTTACTTTGATATCACCTGAAAGA
ATAAATCAGTATTGGGATGGTCACTTGAAGCAAGAACGTATCGTAGCAGAAGCAATTGATAAGGTGGT
TCTAGGAGAAGATGCTCTATTCAATTCGAGTGATAATGAATTAATTCTCAAATGTGGAGGGACACCAA
ATGCACGGAATCTTATCGATATCGAGCCAGTCGCAACTTTCATAGAATTTGAACAACTGATCTGCACA
ATGTTGACAACCCACTTGAAGGAAATAATTGATATAACAAGGTCTGGAACCCAGGATTATGAAAGTTT
ATTACTCACTCCTTACAATTTAGGTCTTCTTGGTAAAATCAGTACGATAGTGAGATTATTAACAGAAAG
GATTCTAAATCATACTATCAGGAATTGGTTGATCCTCCCACCTTCGCTCCGGATGATCGTGAAGCAGGA
CTTGGAATTCGGCATATTCAGGATTACTTCCATCCTCAATTCTGATCGGTTCCTGAAGCTTTCTCCAAAT
AGGAAATACTTGATTACACAATTAACTGCAGGCTACATTAGGAAATTGATTGAGGGGGATTGTAATAT
CGATCTAACCAGACCTATCCAAAAACAAATCTGGAAAGCATTAGGTTGTGTAGTCTATTGTCACGATC
CAGTAGATCAAAGGGAATCAACAGAGTTTATTGATATAAATATTAATGAAGAAATAGACCGCGGGAT
CGATGGCGAGGAAATCTAAATATATCAAGAATCAGAATTAGTTTAAGAAAAAAGAAGAGGATTAATC
TTGGTTTTCCCCTTGGT.

11. CVL59

The nucleic acid sequence for CVL58 is:

(SEQ ID NO: 71)
ACCAGGGGGAAAACGAAGTGGTGACTCAAATCATCGAAGACCCTCGAGATTACATAGGTCCGGAACC
TATGGCCTTCGTGACCGACCTCGAGTCAGAGTAGTTCAATAAGGACCTATCAAGTTTGGGCAATTTTTC
GTCCCCGACACAAAAATGTCATCCGTGCTTAAAGCATATGAGCGATTCACACTCACTCAAGAACTGCA
AGATCAGAGTGAGGAAGGTACAATCCCACCTACAACACTAAAACCGGTAATCAGGGTATTTATACTAA
CCTCTAATAACCCAGAGCTAAGATCCCGGCTTCTTCTATTCTGCCTACGGATTGTTCTCAGTAATGGTG
CAAGGGATTCCCATCGCTTTGGAGCATTACTTACAATGTTTTCGCTACCATCAGCCACAATGCTCAATC
ATGTCAAATTAGCTGACCAGTCACCAGAAGCTGATATCGAAAGGGTAGAGATCGATGGCTTTGAGGA
GGGATCATTCCGCTTAATCCCCAATGCTCGTTCAGGTATGAGCCGTGGAGAGATCAATGCCTATGCTG
CACTTGCAGAAGATCTACCTGACACACTAAACCATGCAACACCTTTTGTTGATTCCGAAGTCGAGGGA
ACTGCATGGGATGAGATTGAGACTTTCTTAGATATGTGTTACAGTGTCCTAATGCAGGCATGGATAGT
GACTTGCAAGTGCATGACTGCGCCAGACCAACCTGCTGCTTCTATTGAGAAACGCCTGCAAAAATATC
GTCAGCAAGGCAGGATCAACCCGAGATATCTCCTGCAACCGGAGGCTCGAAGAATAATCCAGAATGT
AATCCGAAAGGGAATGGTGGTCAGACATTTCCTCACCTTTGAACTGCAGCTTGCCCGAGCACAAAGCC
TTGTATCAAATAGGTATTATGCTATGGTAGGGGATGTTGGAAAGTATATAGAGAATTGTGGAATGGGA
GGCTTCTTTTTGACACTAAAATATGCATTAGGAACCAGATGGCCCACACTTGCTTTAGCTGCATTTTCA
GGAGAGCTAACAAAGCTAAAGTCCCTCATGGCATTATACCAGACCCTTGGTGAGCAGGCCCGATATTT
GGCCCTATTGGAGTCACCACATTTGATGGATTTTGCTGCAGCAAACTACCCACTGCTATATAGCTATGC
TATGGGAATAGGCTATGTGTTAGATGTCAACATGAGGAACTACGCTTTCTCCAGATCATACATGAATA
AGACATATTTCCAATTGGGAATGGAAACTGCAAGAAAACAACAGGGTGCAGTTGACATGAGGATGGC
AGAAGATCTCGGTCTAACTCAAGCCGAACGCACCGAGATGGCAAATACACTTGCCAAATTGACCACA
GCAAATCGAGGGGCAGACACCAGGGGAGGAGTCAACCCGTTCTCATCTATCACTGGGACAACTCAGG
TGCCCGCTGCAGCAACAGGTGACACATTCGAGAGTTACATGGCAGCGGATCGACTGAGGCAGAGATA
TGCTGATGCAGGCACCCACGATGATGAGATGCCACCATTGGAAGAGGAGGAAGAGGACGACACATCT
GCAGGTCCACGCACTGGACTAACTCTTGAACAAGTGGCCTTGGACATCCAGAACGCAGCAGTTGGAGC
TCCCATCCATACAGATGACCTGAATGCCGCACTGGGTGATCTTGACATCTAGACAATTCAGATCCCAA
TCTTAAATCGACACACCTAATTGACCAGTTAGATGGAACTACAGTGGATTCCATAAGGTTCCTGCCTAC
CATCGGCTTTTAAGAAAAAAATAGGCCCGGACGGGTTAGCAACAAGCGACTGCCGATGCCAATAACA
CAATCCACAATCTACAATGGATCCCACTGATCTGAGCTTCTCCCCAGATGAGATCAATAAGCTCATAG
AGACAGGCCTGAATACTGTGGAGTATTTTACTTCCCAACAAGTCACAGGAACATCCTCTCTTGGAAAG
AATACAATACCACCAGGGGTCACAGGACTACTAACCAATGCTGCAGAGGCAAAGATCCAAGAGTCAA
CCAACCATCAGAAGGGTTCAGTTGGTGGGGGCGCAAAACCAAAGAAACCGCGACCAAAAATTGCCAT
TGTGCCAGCAGATGACAAAACGGTGCCCGGAAAGCCGATCCCAAACCCTCTATTAGGTCTGGACTCCA
CCCCGAGCACCCAAACTGTGCTTGATCTAAGTGGGAAAACATTACCATCAGGATCCTATAAGGGGGTT
AAACTTGCGAAATTTGGAAAAGAAAATCTGATGACACGGTTCATCGAGGAACCCAGAGAGAATCCTA
TCGCAACCAATTTCCCCATCGATTTTAAGAGGGGCAGGGATACCGGCGGGTTCCATAGAAGGGAGTAC
TCAATCGGATGGGTGGGAGATGAAGTCAAGGTCACTGAGTGGTGCAATCCATCCTGTTCTCCAATCAC
CGCTGCAGCAAGGCGATTTGAATGCACTTGTCACCAGTGTCCAGTCACTTGCTCTGAATGTGAACGAG
ATACTTAATACAGTGAGAAATTTGGACTCTCGGATGAATCAACTGGAGACAAAAGTAGATCGCATTCT
CTCATCTCAGTCTCTAATCCAGACCATCAAGAATGACATAGTTGGACTTAAAGCAGGGATGGCTACTT
TAGAAGGAATGATTACAACTGTGAAAATCATGGACCCGGGAGTTCCCAGTAATGTTACTGTGGAAGAT
GTACGCAAGAAACTAAGTAACCATGCTGTTGTTGTGCCAGAATCATTCAATGATgccTTCTTGACTCAAT
CTGAAGATGTAATTTCACTTGATGAGTTGGCTCGACCAACTGCAACAAGTGTTAAGAAGATTGTCAGG
AAGGTTCCTCCTCAGAAGGATCTGACTGGATTGAAGATCACACTAGAGCAATTGGCAAAGGATTGCAT
CAGCAAACCGAAGATGAGGGAAGAGTATCTCCTCAAAATCAACCAGGCTTCCAGTGAGGCTCAGCTA
ATTGACCTCAAGAAAGCAATCATCCGCAGTGCAATTTGATCAAGAAACACCCAATTACACTACACTGG
TATGACACTGTACTAACCCTGAGGGTTTTAGAAAAAACGATTAACGATAAATAAGCCCGAACACTACA
CACCACCCGAGGCAGCCATGCCATCCATCAGCATCCCCGCAGACCCCACCAATCCACGTCAATCAATA
AAAGCGTTCCCAATTGTGATCAACCGTGATGGGGGTGAGAAAGGTCGCTTGGTTAAACAACTACGCAC
AACCTACTTGAATGACCTAGATACTCATGAGCCACTGGTGACATTCGTAAATACTTATGGATTCATCTA
CGAACAGGATCGGGGAAATACCATTGTCGGAGAGGATCAACATGGGAAGAAAAGAGAGGCTGTGACT
GCTGCAATGGTTACCCTTGGATGTGGGCCTAATCTACCATCATTAGGGAATGTCCTGGGACAACTGAG
TGAATTCCAGGTCATTGTTAGGAAGACATCCAGCAAAGCGGAAGAGATGGTCTTTGAAATTGTTAAGT
ATCCGAGAATATTTCGGGGTCATACATTAATCCAGAAAGGACTAGTCTGTGTCTCCGCAGAAAAATTT
GTTAAGTCACCAGGGAAAGTACAATCTGGAATGGACTATCTCTTCATTCCGACATTTCTGTCAGTGACT
TACTGTCCAGCTGCAATCAAATTTCAGGTACCTGGCCCCATGTTGAAAATGAGATCAAGATACACTCA
GAGCTTACAACTTGAACTAATGATAAGAATCCTGTGTAAGCCCGATTCGCCACTTATGAAGGTCCATA
TCCCTGACAAGGAAGGAAGAGGATGTCTTGTATCAGTATGGTTGCATGTATGCAATATCTTCAAATCA
GGAAACAAGAATGGCAGTGAGTGGCAGGAATACTGGATGAGAAAGTGTGCTAACATGCAACTTGAAG
TGTCGATTGCAGATATGTGGGGACCAACTATCATAATTCATGCCAGAGGTCACATTCCCAAAAGTGCT
AAGTTGTTTTTTGGAAAGGGTGGATGGAGCTGCCATCCACTTCACGAAGTTGTTCCAAGTGTCACTAA
AACACTATGGTCCGTGGGCTGTGAGATTACAAAGGCGAAGGCAATAATACAAGAGAGTAGCATCTCT
CTTCTCGTGGAGACTACTGACATCATAAGTCCAAAAGTCAAAATTTCATCTAAGCATCGCCGCTTTGGG
AAATCAAATTGGGGTCTGTTCAAGAAAACCAAATCACTGCCCAACCTGACGGAGCTGGAATGACTGAC
CCCCAATCGAGACTACACCACCTCAAACTATAGGTGGGTGGTACCTCAGTGATTAATCTCGTAAGCAC
TGATCGTAGGCTACAACACACTAATATTATCCAGATTAGAGAGCTTAATTAGCTCTGTATTAATAATAA
CACTACTATTCCAATAACTGGAATCACCAGCTTGATTTATCTCCAAAATGATTCAAAGAAAACAAATC
ATATTAAGACTATCCTAAGCACGAACCCATATCGTCCTTCAAATCATGGGTACTATAATTCAATTTCTG
GTGGTCTCCTGTCTATTGGCAGGAGCAGGCAGTCTTGATCCAGCAGCCCTCATGCAAATCGGTGTCATT
CCAACAAATGTCCGGCAACTTATGTATTATACTGAGGCCTCATCAGCATTCATTGTTGTGAAGTTAATG
CCTACAATTGACTCGCCGATTAGTGGATGTAATATAACATCAATTTCAAGCTATAATGCAACAGTGAC
AAAACTCCTACAGCCGATCGGTGAGAATTTGGAGACGATTAGGAACCAGTTGATTCCAACTCGGAGGA
GACGCCGGTTTGCAGGGGTGGTGATTGGATTAGCTGCATTAGGAGTAGCTACTGCCGCACAGGTCACT
GCCGCAGTAGCACTAGTAAAGGCAAATGAAAATGCTGCGGCTATACTCAATCTCAAAAATGCAATCCA
AAAAACAAATGCAGCAGTTGCAGATGTGGTCCAGGCCACACAATCACTAGGAACaGCAGTTCAAGCA
GTTCAAGATCACATAAACAGTGTGGTAAGTCCAGCAATTACAGCAGCCAATTGTAAAGCCCAAGATGC
TATCATTGGCTCAATCCTCAATCTCTATTTGACCGAGTTGACAACTATCTTCCACAATCAAATTACAAA
CCCTGCATTGAGTCCTATTACAATTCAAGCTTTAAGGATCCTACTAGGGAGTACCTTGCCGACTGTGGT
CGAAAAATCTTTCAATACCCAGATAAGTGCGGCTGAGCTTCTCTCATCAGGGTTATTGACAGGCCAGA
TTGTGGGATTAGATTTGACCTATATGCAGATGGTCATAAAAATTGAGCTGCCAACTTTAACTGTACAAC
CTGCAACCCAGATCATAGATCTGGCCACCATTTCTGCATTCATTAACAATCAAGAAGTCATGGCCCAA
TTACCAACACGTGTTATTGTGACTGGCAGCTTGATCCAAGCCTATCCCGCATCGCAATGCACTATTACA
CCCAACACTGTGTACTGTAGGTATAATGATGCCCAAGTACTCTCAGATGATACGATGGCTTGCCTCCA
AGGTAACTTGACAAGATGCACCTTCTCTCCAGTGGTTGGGAGCTTTCTCACTCGATTCGTGCTGTTCGA
TGGAATAGTTTATGCAAATTGCAGGTCGATGTTGTGCAAGTGCATGCAGCCTGCTGCTGTGATCCTACA
GCCGAGTTCATCCCCTGTAACTGTCATTGACATGTACAAATGTGTGAGTCTGCAGCTTGACAATCTCAG
ATTCACCATCACTCAATTGGCCAATGTAACCTACAATAGCACCATCAAGCTTGAAACATCCCAGATCTT
GCCTATTGATCCGTTGGATATATCCCAGAATCTAGCTGCGGTGAATAAGAGTCTAAGTGATGCACTAC
AACACTTAGCACAAAGTGACACATACCTTTCTGCAATCACATCAGCTACGACTACAAGTGTATTATCC
ATAATAGCAATCTGTCTTGGATCGTTAGGTTTAATATTAATAATCTTGCTCAGTGTAGTTGTGTGGAAG
TTATTGACCATTGTCGCTGCTAATCGAAATAGAATGGAGAATTTTGTTTATCATAATTCAGCATTCCAC
CACTCACGATCTGATCTCAGTGAGAAAAATCAACCTGCAACTCTTGGAACAAGATAAGACAGTCATCC
ATTAGTAATTTTTAAGAAAAAAACGATAGGACCGAAACTAGTATTGAAAGAACCGTCTCGGTCAATCT
AGGTAATCGAGCTGcTACCGTCTCGGAAAGCTCAAATCgcgcgctaacgtacgCACCTGCTATAGGCTATCCAC
TGCATCATCTCTTTTTAAGAAAAAAATAGGCCCGGACGGGTTAGCAACAAGCGACTGCCGGTGCCAAC
AACACAATCCACAATCTACAGTCGACGCGGCCGCCACCatgttcgtgttcctggtgctgctgccactggtc
agcagccagtgcgtgaatttcactacccggacccagctgcctcctgcttatacaaacagcttcaccaggggagtg
tactaccccgataaggtgttccgcagctctgtgctgcacagcacccaggacttgtttcttcccttctt
cagcaacgtgacctggttccacgccatccacgtgtccggcaccaacggcaccaaaagattcgccaatcctgtg
ctgcctttcaacgatggcgtgtactttgcctctacagagaagtctaacatcatcagaggctggatcttcggc
acaaccctggattctaaaacccagagcctgctgatcgtgaacaatgccaccaatgtagtgatca
aggtttgtgagttccagttctgcaacgacccctttctgggcgtttactaccacaagaacaacaaaagctg
gatggaaagcgagttcagggtgtactctagcgccaacaactgtaccttcgagtacgtgagccagcctttt
ctgatggacctggagggcaagcagggcaacttcaaaaacctgcgggagttcgtgtttaagaacatcgacggata
cttcaagatttactccaagcacaccccaattaacttggttagaggactgccccaaggcttcagcgccctggaa
cccctggtggacctgcctatcggcatcaacatcacaagattccaaaccctgcatagaagctatctgac
accaggcgactctagctctggatggacagccggcgccgctgcttattacgtgggctacctgcagcctagaa
ccttcctcctgaaatacaacgagaacggaaccatcaccgacgccgtggactgcgccctggatcctctga
gcgagacaaagtgcaccctgaagtccttcaccgtcgaaaagggcatctaccaaacaagcaacttcagagtgc
agcctactgagagcatcgtgcggtttccaaacatcaccaacctgtgtccttttggcgaggtgttcaacgc
cacccggttcgcttccgtgtacgcctggaatagaaagcggatcagcaactgcgtggccgactacagcgtgctg
tataatagcgcctcattttctacttttaaatgttacggcgtgtctcctacaaagctgaacgatctgtgcttcac
aaatgtgtacgccgatagcttcgtgattagaggcgacgaggtgcggcagatcgcccctggccagaccggcaa
catcgctgactacaactacaagctgcctgacgacttcaccggctgtgtgatcgcatggaacagcaataacct
ggactctaaggtgggcggaaactacaattacctgtacagactgttcagaaagagcaacctgaagcccttcgagagag
atatctccacagaaatctatcaggccggatctacaccttgtaatggcgtgaagggcttcaactgctacttccc
tctgcaatcctacggctttcagcccacatacggcgtgggctaccagccataccgggtcgtggtgctgagcttc
gagctgctgcacgcccctgccacagtgtgcggccctaagaagagcaccaatctggtaaagaacaagtgcgtcaact
tcaatttcaacggcctgaccgggaccggagtgctgaccgaaagcaacaagaaattcctgccgttccagcagttcg
gcagagatatcgccgacaccaccgatgccgtgcgggacccccagacacttgaaatcctggacatcaccccttgcagtttt
ggcggcgtgtccgtgatcacacctggaacaaacaccagcaaccaggtggccgtgctgtaccagggcgtcaactgca
ccgaggtccccgtggccatccacgccgatcagctgacacctacatggcgggtgtactctaccggcagcaacgtgt
tccaaaccagagccggctgcctgatcggcgctgagcacgtgaacaacagctacgagtgcgacattcctatcgg
tgctggcatctgcgcctcttaccagacacagacaaacagccccagaagagcgagaagcgtggctagtcagagcatca
tcgcctacaccatgagcctgggcgtggagaacagcgtggcctactctaacaatagcatcgccatcccaacca
acttcacaatctctgtgaccacagagatcctgcccgtgtccatgaccaagacatctgtggactgcacaatgtatat
ctgtggcgattctaccgagtgcagcaacctgctgctccagtacggcagcttttgtacccagctgaatagagccctgac
aggcatcgccgtggagcaggataagaacacacaggaggtgttcgcccaggtgaagcaaatctacaagaccccccctatca
aggactttggcggcttcaatttttcccagatcctgcctgatccatccaagccttctaagcggagctttatcgaggacctg
ctgttcaacaaggtgaccctggccgatgccggcttcatcaagcagtatggcgattgcctgggcgacatcgcagccaggg
acctgatctgcgcccagaagtttaatggcctgaccgtgctgccacccctgctgacagatgagatgatcgcacagtacac
aagcgccctgctggccggcaccatcacatccggatggaccttcggcgcaggagccgccctccagatcccctttgccat
gcagatggcctataggttcaacggcatcggcgtgacccagaatgtgctgtacgagaaccagaagctgatcgccaat
cagtttaactccgccatcggcaagatccaggacagcctgtcctctacagccagcgccctgggcaagctccaggatgtgg
tgaatcagaacgcccaggccctgaataccctggtgaagcagctgagcagcaacttcggcgccatctctagcgtgctgaat
gacatcctgagccggctggacaaggtggaggcagaggtgcagatcgaccggctgatcaccggccggctccagag
cctccagacctatgtgacacagcagctgatcagggccgccgagatcagggccagcgccaatctggcagcaaccaaga
tgtccgagtgcgtgctgggccagtctaagagagtggacttttgtggcaagggctatcacctgatgtccttccctcagtc
tgccccacacggcgtggtgtttctgcacgtgacctacgtgcccgcccaggagaagaacttcaccacagcccctgcc
atctgccacgatggcaaggcccactttccaagggagggcgtgttcgtgtccaacggcacccactggtttgtgacac
agcgcaatttctacgagccccagatcatcaccacagacaacaccttcgtgagcggcaactgtgacgtggtcat
cggcatcgtgaacaataccgtgtatgatccactccagcccgagctggacagctttaaggaggagctggataagta
tttcaagaatcacacctcccctgacgtggatctgggcgacatcagcggcatcaatgcctccgtggtgaacatccag
aaggagatcgaccgcctgaacgaggtggctaagaatctgaacgagagcctgatcgacctccaggagctgggcaagtatga
gcagtacatcaagtggccctggtacatctggctgggcttcatcgccggcctgatcgccatcgtgatggtgaccatcatgc
tgtgctgtatgacatcctgctgttcttgcctgaagggctgctgtagctgtggctcctgctgtAAGTTATTGACCATTGTC
GTTGCTAATCGAAATAGAATGGAGAATTTTGTTTATCATAAATGAACGCGTGCTAGCCTCCTGCCATAC
TTCCTACTCACATCATATCTATTTTAAAGAAAAAATAGGCCCGAACACTAATCGTGCCGGCAGTGCCA
CTGCACACACAACACTACACATACAATACACTACAATGGTTGCAGAAGATGCCCCTGTTAGGGGCACT
TGCCGAGTATTATTTCGAACAACAACTTTAATTTTTCTATGCACACTATTAGCATTAAGCATCTCTATCC
TTTATGAGAGTTTAATAACCCAAAAGCAAATCATGAGCCAAGCAGGCTCAACTGGATCTAATTCTAGA
TTAGGAAGTATCACTGATCTTCTTAATAATATTCTCTCTGTCGCAAATCAGATTATATATAACTCTGCA
GTCGCTCTACCTCTACAATTGGACACTCTTGAATCAACACTCCTTACAGCCATTAAGTCTCTTCAAACA
AGTGACAAGCTAGAACAGAACTGCTCGTGGGGTGCTGCACTGATTAATGATAATAGATACATTAATGG
CATCAATCAGTTCTATTTCTCAATTGCTGAGGGTCGCAAGCTGACACTTGGCCCACTTCTTAATATACC
TAGTTTCATTCCAACTGCCACGACACCAGAGGGCTGCACCAGGATCCCATCATTCTCGCTCACCAAGA
CACACTGGTGTTATACACACAATGTTATCCTGAATGGATGCCAGGATCATGTATCCTCAAATCAATTTG
TTTCCATGGGAATCATTGAACCCACTTCTGCCGGGTTTCCATCCTTTCGAACCCTAAAGACTCTATATC
TCAGCGATGGGGTCAATCGTAAGAGCTGCTCTATCAGTACAGTTCCGGGGGGTTGTATGATGTACTGT
TTCGTCTCTACTCAACCAGAGAGGGATGACTACTTTTCTACCGCTCCTCCAGAACAACGAATTATTATA
ATGTACTATAATGATACAATCGTGGAGCGCATAATTAATCCACCCGGGGTACTAGATGTATGGGCAAC
ATTGAACCCAGGAACAGGAAGCGGGGTATATTATTTAGGTTGGGTACTCTTTCCAATATATGGCGGCG
TGATTAAAGATACGAGTTTATGGAATAATCAAGCAAATAAATACTTTATCCCCCAGATGGTTGCTGCT
CTCTGCTCACAAAACCAGGCAACTCAAGTCCAAAATGCTAAGTCATCATACTATAGCAGCTGGTTTGG
CAATCGAATGATTCAGTCTGGGATCCTGGCATGTCCTCTTCAACAGGATCTAACCAATGAGTGTTTAGT
TCTGCCCTTTTCTAATGATCAGGTGCTTATGGGTGCTGAAGGGAGATTATACATGTATGGTGACTCGGT
GTATTACTACCAAAGAAGCAATAGTTGGTGGCCTATGACCATGCTGTATAAGGTAACCATAACATTCA
CTAATGGTCAGCCATCTGCTATATCAGCTCAGAATGTGCCCACACAGCAGGTCCCTAGACCTGGGACA
GGAAGCTGCTCTGCAACAAATAGATGTCCCGGTTTTTGCTTGAAAGGAGTGTATGCTGATGCCTGGTT
ACTGACCAACCCTTCGTCTACCAGTACATTTGGATCAGAAGCAACCTTCACTGGTTCTTATCTCAACGC
AGCAACTCAGCGTATCAATCCGACGATGTATATCGCGAACAACACACAGATCATAAGCTCACAGCAAT
TTGGATCAAGCGGTCAAGAAGCAGCATATGGCCACACAACTTGTTTTAGGGACACAGGCTCTGTTATG
GTATACTGTATCTATATTATTGAATTGTCCTCATCTCTCTTAGGACAATTTCAGATTGTCCCATTTATCC
GTCAGGTGACACTATCCTAAAGGCAGAAGCCTCCAGGTCTGACCCAGCCAATCAAAGCATTATACCAG
ACCATGGAATGCATACCAAACATTATTGACACTAATGACACACAAAATTGGTTTTAAGAAAAACCAAG
AGAACAATAGGCCAGAATGGCTGGGTCTCGGGAGATATTACTCCCTGAAGTCCATCTCAATTCACCAA
TTGTAAAGCATAAGCTATACTATTACATTCTACTTGGAAACCTCCCAAATGAGATCGACATTGACGATT
TAGGTCCATTACATAATCAAAATTGGAATCAAATAGCACATGAAGAGTCTAACTTAGCCCAACGCTTG
GTAAATGTAAGAAATTTTCTAATTACCCACATCCCTGATCTTAGAAAGGGCCATTGGCAAGAGTATGT
CAATGTAATACTGTGGCCGCGAATTCTTCCCTTGATCCCGGATTTTAAAATCAATGACCAATTGCCTCT
ACTCAAAAATTGGGACAAGTTAGTTAAAGAATCATGTTCAGTAATCAATGCGGGTACTTCCCAGTGCA
TTCAGAATCTCAGCTATGGACTGACAGGTCGTGGGAACCTCTTTACACGATCACGTGAACTCTCTGGTG
ACCGCAGGGATATTGATCTTAAGACGGTTGTGGCAGCATGGCATGACTCAGACTGGAAAAGAATAAG
TGATTTTTGGATTATGATCAAATTCCAGATGAGACAATTAATTGTTAGGCAAACAGATCATAATGATCC
TGATTTAATCACGTATATCGAAAATAGAGAAGGCATAATCATCATAACCCCTGAACTGGTAGCATTAT
TTAACACTGAGAATCATACACTAACATACATGACCTTTGAAATTGTACTGATGGTTTCAGATATGTACG
AAGGTCGTCACAACATTTTATCACTATGCACAGTTAGCACTTACCTGAATCCTCTGAAGAAAAGAATA
ACATATTTATTGAGCCTTGTAGATAACTTAGCTTTTCAGATAGGTGATGCTGTATATAACATAATTGCT
TTGCTAGAATCCTTTGTATATGCACAGTTGCAAATGTCAGATCCCATCCCAGAACTCAGAGGACAATTC
CATGCATTCGTATGTTCTGAGATTCTTGATGCACTAAGGGGAACTAATAGTTTCACCCAGGATGAATCA
AGAACTGTGACAACCAATTTGATATCCCCATTCCAAGATCTGACCCCAGATCTTACGGCTGAATTGCTC
TGTATAATGAGGCTTTGGGGACACCCCATGCTCACCGCCAGTCAAGCTGCGGGAAAGGTACGCGAGTC
CATGTGTGCTGGAAAAGTATTAGACTTTCCCACCATTATGAAAACACTAGCCTTTTTCCATACTATTCT
GATCAATGGATACAGGAGGAAGCATCATGGAGTATGGCCACCCTTAAACTTACCGGGTAATGCTTCAA
AGGGTCTCACGGAACTTATGAATGACAATACTGAGATAAGCTATGAATTCACACTTAAGCATTGGAAG
GAAATCTCTCTTATAAAATTCAAGAAATGTTTTGATGCAGACGCAGGTGAGGAACTCAGTATATTTAT
GAAAGATAAAGCAATTAGTGCCCCAAAACAAGATTGGATGAGTGTGTTTAGAAGAAGCCTAATCAAA
CAGCGCCATCAGCATCATCAGGTCCCCCTACCAAATCCATTCAATCGACGGCTATTGCTAAACTTTCTC
GGAGATGACAAATTCGACCCGAATGTGGAGCTACAGTATGTAACATCAGGTGAGTATCTACATGATGA
CACGTTTTGTGCATCATATTCACTAAAAGAGAAGGAAATTAAACCTGATGGTCGAATTTTTGCAAAGT
TGACTAAGAGAATGAGATCATGTCAAGTTATAGCAGAATCTCTTTTAGCGAACCATGCTGGGAAGTTA
ATGAAAGAGAATGGTGTTGTGATGAATCAGCTATCATTAACAAAATCACTATTAACAATGAGTCAGAT
TGGAATAATATCCGAGAAAGCTAGAAAATCGACTCGAGATAACATAAATCAACCTGGTTTCCAGAATA
TCCAGAGAAATAAATCACATCACTCCAAGCAAGTCAATCAGCGAGATCCAAGTGATGACTTTGAATTG
GCAGCATCTTTTTTAACTACTGATCTCAAAAAATATTGTTTACAATGGAGGTACCAGACAATTATCCCA
TTTGCTCAATCATTAAACAGAATGTATGGTTATCCTCATCTCTTTGAGTGGATTCACTTACGGCTAATG
CGTAGTACACTTTACGTGGGGGATCCCTTCAACCCACCAGCAGATACCAGTCAATTTGATCTAGATAA
AGTAATTAATGGAGATATCTTCATTGTATCACCCAGAGGTGGAATTGAAGGGCTATGTCAAAAGGCTT
GGACAATGATATCTATCTCTGTGATAATTCTATCTGCCACAGAGTCTGGCACACGAGTAATGAGTATG
GTGCAGGGAGATAATCAAGCAATTGCTGTCACCACACGAGTACCAAGGAGCCTGCCGACTCTTGAGA
AAAAGACTATTGCTTTTAGATCTTGTAATCTATTCTTTGAGAGGTTAAAATGTAATAATTTTGGATTAG
GTCACCATTTGAAAGAACAAGAGACTATCATTAGTTCTCACTTCTTTGTTTATAGCAAGAGAATATTCT
ATCAGGGGAGGATTCTAACGCAAGCCTTAAAAAATGCTAGTAAGCTCTGCTTGACAGCTGATGTCCTA
GGAGAATGTACCCAATCATCATGTTCTAATCTTGCAACTACTGTCATGAGGTTAACTGAGAATGGTGTT
GAAAAAGATATCTGTTTCTACTTGAATATCTATATGACCATCAAACAGCTCTCCTATGATATCATCTTC
CCTCAAGTGTCAATTCCTGGAGATCAGATCACATTAGAATACATAAATAATCCACACCTGGTATCACG
ATTGGCTCTTCTGCCATCCCAGCTAGGAGGTCTAAACTACCTGTCATGCAGTAGGCTGTTCAATCGAAA
CATAGGCGACCCGGTGGTTTCCGCAGTTGCAGATCTTAAGAGATTAATTAAATCAGGATGTATGGATT
ACTGGATCCTTTATAACTTATTAGGGAGAAAACCGGGAAACGGCTCATGGGCTACTTTAGCAGCTGAC
CCGTACTCAATCAATATAGAGTATCAATACCCCCCAACTACAGCTCTTAAGAGGCACACCCAACAAGC
TCTGATGGAACTCAGTACGAATCCAATGTTACGTGGCATATTCTCTGACAATGCACAGGCAGAAGAAA
ATAATCTTGCTAGATTTCTCCTGGATAGGGAGGTGATCTTTCCGCGTGTAGCTCACATCATCATTGAGC
AAACCAGTGTCGGGAGGAGAAAACAGATTCAAGGATATTTGGATTCAACTAGATCGATAATGAGTAA
ATCACTAGAAATTAAGCCCTTGTCCAATAGGAAGCTTAATGAAATACTGGATTACAACATCAATTACC
TAGCTTACAATTTGGCATTACTCAAGAATGCTATTGAACCTCCGACTTATTTGAAAGCAATGACTCTTG
AAACATGTAGCATCGACATTGCAAGGAGCCTCCGGAAGCTCTCCTGGGCCCCACTCTTGGGTGGGAGA
AATCTTGAAGGATTAGAGACGCCAGATCCCATTGAAATTACTGCAGGAGCATTAATTGTTGGATCGGG
CTACTGTGAACAGTGTGCTGCAGGAGACAATCGATTCACATGGTTTTTCTTGCCATCTGGTATCGAGAT
AGGAGGGGATCCCCGTGATAATCCTCCTATCCGTGTACCGTACATTGGCTCCAGGACTGATGAGAGGA
GGGTAGCCTCAATGGCATACATCAGGGGTGCCTCGAGTAGCCTAAAAGCAGTTCTTAGACTGGCGGGA
GTGTACATCTGGGCATTCGGAGATACTCTGGAGAATTGGATAGATGCACTGGATTTGTCTCACACTAG
AGTTAACATCACACTTGAACAGCTGCAATCCCTCACCCCACTTCCAACCTCTGCCAATCTAACCCATCG
GTTGGATGATGGCACAACTACCCTAAAGTTTACTCCTGCGAGCTCTTACACCTTTTCAAGTTTCACTCA
TATATCAAATGATGAGCAATACCTGACAATTAATGACAAAACTGCAGATTCAAATATAATCTACCAAC
AGTTAATGATCACTGGACTCGGAATTTTAGAAACATGGAATAATCCCCCAATCAATAGAACATTCGAA
GAATCTACCCTACATTTGCACACTGGTGCATCATGTTGTGTCCGACCTGTGGACTCCTGCATCATCTCA
GAAGCATTAACAGTCAAGCCACATATTACAGTACCGTACAGCAATAAATTTGTATTTGATGAAGACCC
GCTATCTGAATATGAGACTGCAAAACTGGAATCGTTATCATTTCAAGCCCAATTAGGCAACATTGATG
CTGTAGATATGACAGGTAAATTAACATTATTGTCCCAATTCACTGCAAGGCAGATTATTAATGCAATC
ACTGGACTCGATGAGTCTGTCTCTCTTACTAATGATGCCATTGTTGCATCAGACTATGTCTCCAATTGG
ATTAGTGAATGCATGTATACCAAATTAGATGAATTATTTATGTATTGTGGGTGGGAACTACTATTGGAA
CTATCCTATCAAATGTATTATCTGAGGGTAGTTGGGTGGAGTAATATAGTGGATTATTCTTACATGATC
TTGAGAAGAATCCCGGGTGCAGCATTAAACAATCTGGCATCTACATTAAGTCATCCAAAACTTTTCCG
ACGAGCTATCAACCTAGATATAGTTGCCCCCTTAAATGCTCCTCATTTTGCATCTCTGGACTACATCAA
GATGAGTGTGGATGCAATACTCTGGGGCTGTAAAAGAGTCATCAATGTGCTCTCCAATGGAGGGGACT
TAGAATTAGTTGTGACATCTGAAGATAGCCTTATTCTCAGTGACCGATCCATGAATCTCATTGCAAGGA
AATTAACTTTATTATCACTGATTCACCATAATGGTTTGGAACTACCAAAGATTAAGGGGTTCTCTCCTG
ATGAGAAGTGTTTCGCTTTGACAGAATTTTTGAGGAAAGTGGTGAACTCAGGGTTGAGTTCAATAGAG
AACCTATCAAATTTTATGTACAATGTGGAGAACCCACGGCTTGCAGCATTCGCCAGCAACAATTACTA
CCTGACCAGAAAATTATTGAATTCAATACGAGATACTGAGTCGGGTCAAGTAGCAGTCACCTCATATT
ATGAATCATTAGAATATATTGATAGTCTTAAGCTAACCCCACATGTGCCTGGTACCTCATGCATTGAGG
ATGATAGTCTATGTACAAATGATTACATAATCTGGATCATAGAGTCTAATGCAAACTTGGAGAAGTAT
CCAATTCCAAATAGCCCTGAGGATGATTCCAATTTCCATAACTTTAAGTTGAATGCTCCATCGCACCAT
ACCTTACGCCCATTAGGGTTGTCATCAACTGCTTGGTATAAGGGTATAAGCTGTTGCAGGTACCTTGAG
CGATTAAAGCTACCACAAGGTGATCATTTATATATTGCAGAAGGTAGTGGTGCCAGTATGACAATCAT
AGAATACCTATTCCCAGGAAGAAAGATATATTACAATTCTTTATTTAGTAGTGGTGACAATCCCCCAC
AAAGAAATTATGCACCAATGCCTACTCAGTTCATTGAGAGTGTCCCATACAAGCTCTGGCAAGCACAC
ACAGATCAATATCCCGAGATTTTTGAGGACTTCATCCCTCTATGGAACGGAAATGCCGCCATGACTGA
CATAGGAATGACAGCTTGTGTAGAATTCATCATCAATCGAGTCGGCCCAAGGACTTGCAGTTTAGTAC
ATGTAGATTTGGAATCAAGTGCAAGCTTAAATCAACAATGCCTGTCAAAGCCGATAATTAATGCTATC
ATCACTGCTACAACTGTTTTGTGCCCTCATGGGGTGCTTATTCTGAAATATAGTTGGTTGCCATTTACTA
GATTTAGTACTTTGATCACTTTCTTATGGTGCTACTTTGAGAGAATCACTGTTCTTAGGAGCACATATTC
TGGTCCAGCTAATCATGAGGTTTATTTAATTTGTATCCTTGCCAACAACTTTGCATTCCAGACTGTCTCG
CAGGCAACAGGAATGGCGATGACTTTAACCGATCAAGGGTTTACTTTGATATCACCTGAAAGAATAAA
TCAGTATTGGGATGGTCACTTGAAGCAAGAACGTATCGTAGCAGAAGCAATTGATAAGGTGGTTCTAG
GAGAAGATGCTCTATTCAATTCGAGTGATAATGAATTAATTCTCAAATGTGGAGGGACACCAAATGCA
CGGAATCTTATCGATATCGAGCCAGTCGCAACTTTCATAGAATTTGAACAACTGATCTGCACAATGTTG
ACAACCCACTTGAAGGAAATAATTGATATAACAAGGTCTGGAACCCAGGATTATGAAAGTTTATTACT
CACTCCTTACAATTTAGGTCTTCTTGGTAAAATCAGTACGATAGTGAGATTATTAACAGAAAGGATTCT
AAATCATACTATCAGGAATTGGTTGATCCTCCCACCTTCGCTCCGGATGATCGTGAAGCAGGACTTGG
AATTCGGCATATTCAGGATTACTTCCATCCTCAATTCTGATCGGTTCCTGAAGCTTTCTCCAAATAGGA
AATACTTGATTACACAATTAACTGCAGGCTACATTAGGAAATTGATTGAGGGGGATTGTAATATCGAT
CTAACCAGACCTATCCAAAAACAAATCTGGAAAGCATTAGGTTGTGTAGTCTATTGTCACGATCCAGT
AGATCAAAGGGAATCAACAGAGTTTATTGATATAAATATTAATGAAGAAATAGACCGCGGGATCGAT
GGCGAGGAAATCTAAATATATCAAGAATCAGAATTAGTTTAAGAAAAAAGAAGAGGATTAATCTTGG
TTTTCCCCTTGGT.

12. CVL60

The nucleic acid sequence for CVL60 is:

(SEQ ID NO: 72)
ACCAGGGGGAAAACGAAGTGGTGACTCAAATCATCGAAGACCCTCGAGATTACATAGGTCCGGAACC
TATGGCCTTCGTGACCGACCTCGAGTCAGAGTAGTTCAATAAGGACCTATCAAGTTTGGGCAATTTTTC
GTCCCCGACACAAAAATGTCATCCGTGCTTAAAGCATATGAGCGATTCACACTCACTCAAGAACTGCA
AGATCAGAGTGAGGAAGGTACAATCCCACCTACAACACTAAAACCGGTAATCAGGGTATTTATACTAA
CCTCTAATAACCCAGAGCTAAGATCCCGGCTTCTTCTATTCTGCCTACGGATTGTTCTCAGTAATGGTG
CAAGGGATTCCCATCGCTTTGGAGCATTACTTACAATGTTTTCGCTACCATCAGCCACAATGCTCAATC
ATGTCAAATTAGCTGACCAGTCACCAGAAGCTGATATCGAAAGGGTAGAGATCGATGGCTTTGAGGA
GGGATCATTCCGCTTAATCCCCAATGCTCGTTCAGGTATGAGCCGTGGAGAGATCAATGCCTATGCTG
CACTTGCAGAAGATCTACCTGACACACTAAACCATGCAACACCTTTTGTTGATTCCGAAGTCGAGGGA
ACTGCATGGGATGAGATTGAGACTTTCTTAGATATGTGTTACAGTGTCCTAATGCAGGCATGGATAGT
GACTTGCAAGTGCATGACTGCGCCAGACCAACCTGCTGCTTCTATTGAGAAACGCCTGCAAAAATATC
GTCAGCAAGGCAGGATCAACCCGAGATATCTCCTGCAACCGGAGGCTCGAAGAATAATCCAGAATGT
AATCCGAAAGGGAATGGTGGTCAGACATTTCCTCACCTTTGAACTGCAGCTTGCCCGAGCACAAAGCC
TTGTATCAAATAGGTATTATGCTATGGTAGGGGATGTTGGAAAGTATATAGAGAATTGTGGAATGGGA
GGCTTCTTTTTGACACTAAAATATGCATTAGGAACCAGATGGCCCACACTTGCTTTAGCTGCATTTTCA
GGAGAGCTAACAAAGCTAAAGTCCCTCATGGCATTATACCAGACCCTTGGTGAGCAGGCCCGATATTT
GGCCCTATTGGAGTCACCACATTTGATGGATTTTGCTGCAGCAAACTACCCACTGCTATATAGCTATGC
TATGGGAATAGGCTATGTGTTAGATGTCAACATGAGGAACTACGCTTTCTCCAGATCATACATGAATA
AGACATATTTCCAATTGGGAATGGAAACTGCAAGAAAACAACAGGGTGCAGTTGACATGAGGATGGC
AGAAGATCTCGGTCTAACTCAAGCCGAACGCACCGAGATGGCAAATACACTTGCCAAATTGACCACA
GCAAATCGAGGGGCAGACACCAGGGGAGGAGTCAACCCGTTCTCATCTATCACTGGGACAACTCAGG
TGCCCGCTGCAGCAACAGGTGACACATTCGAGAGTTACATGGCAGCGGATCGACTGAGGCAGAGATA
TGCTGATGCAGGCACCCACGATGATGAGATGCCACCATTGGAAGAGGAGGAAGAGGACGACACATCT
GCAGGTCCACGCACTGGACTAACTCTTGAACAAGTGGCCTTGGACATCCAGAACGCAGCAGTTGGAGC
TCCCATCCATACAGATGACCTGAATGCCGCACTGGGTGATCTTGACATCTAGACAATTCAGATCCCAA
TCTTAAATCGACACACCTAATTGACCAGTTAGATGGAACTACAGTGGATTCCATAAGGTTCCTGCCTAC
CATCGGCTTTTAAGAAAAAAATAGGCCCGGACGGGTTAGCAACAAGCGACTGCCGATGCCAATAACA
CAATCCACAATCTACAATGGATCCCACTGATCTGAGCTTCTCCCCAGATGAGATCAATAAGCTCATAG
AGACAGGCCTGAATACTGTGGAGTATTTTACTTCCCAACAAGTCACAGGAACATCCTCTCTTGGAAAG
AATACAATACCACCAGGGGTCACAGGACTACTAACCAATGCTGCAGAGGCAAAGATCCAAGAGTCAA
CCAACCATCAGAAGGGTTCAGTTGGTGGGGGCGCAAAACCAAAGAAACCGCGACCAAAAATTGCCAT
TGTGCCAGCAGATGACAAAACGGTGCCCGGAAAGCCGATCCCAAACCCTCTATTAGGTCTGGACTCCA
CCCCGAGCACCCAAACTGTGCTTGATCTAAGTGGGAAAACATTACCATCAGGATCCTATAAGGGGGTT
AAACTTGCGAAATTTGGAAAAGAAAATCTGATGACACGGTTCATCGAGGAACCCAGAGAGAATCCTA
TCGCAACCAATTCCCCCATCGATTTTAAGAGGGGCAGGGATACCGGCGGGTTCCATAGAAGGGAGTAC
TCAATCGGATGGGTGGGAGATGAAGTCAAGGTCACTGAGTGGTGCAATCCATCCTGTTCTCCAATCAC
CGCTGCAGCAAGGCGATTTGAATGCACTTGTCACCAGTGTCCAGTCACTTGCTCTGAATGTGAACGAG
ATACTTAATACAGTGAGAAATTTGGACTCTCGGATGAATCAACTGGAGACAAAAGTAGATCGCATTCT
CTCATCTCAGTCTCTAATCCAGACCATCAAGAATGACATAGTTGGACTTAAAGCAGGGATGGCTACTT
TAGAAGGAATGATTACAACTGTGAAAATCATGGACCCGGGAGTTCCCAGTAATGTTACTGTGGAAGAT
GTACGCAAGAAACTAAGTAACCATGCTGTTGTTGTGCCAGAATCATTCAATGATAGTTTCTTGACTCAA
TCTGAAGATGTAATTTCACTTGATGAGTTGGCTCGACCAACTGCAACAAGTGTTAAGAAGATTGTCAG
GAAGGTTCCTCCTCAGAAGGATCTGACTGGATTGAAGATCACACTAGAGCAATTGGCAAAGGATTGCA
TCAGCAAACCGAAGATGAGGGAAGAGTATCTCCTCAAAATCAACCAGGCTTCCAGTGAGGCTCAGCT
AATTGACCTCAAGAAAGCAATCATCCGCAGTGCAATTTGATCAAGAAACACCCAATTACACTACACTG
GTATGACACTGTACTAACCCTGAGGGTTTTAGAAAAAACGATTAACGATAAATAAGCCCGAACACTAC
ACACCACCCGAGGCAGCCATGCCATCCATCAGCATCCCCGCAGACCCCACCAATCCACGTCAATCAAT
AAAAGCGTTCCCAATTGTGATCAACCGTGATGGGGGTGAGAAAGGTCGCTTGGTTAAACAACTACGCA
CAACCTACTTGAATGACCTAGATACTCATGAGCCACTGGTGACATTCGTAAATACTTATGGATTCATCT
ACGAACAGGATCGGGGAAATACCATTGTCGGAGAGGATCAACATGGGAAGAAAAGAGAGGCTGTGA
CTGCTGCAATGGTTACCCTTGGATGTGGGCCTAATCTACCATCATTAGGGAATGTCCTGGGACAACTG
AGTGAATTCCAGGTCATTGTTAGGAAGACATCCAGCAAAGCGGAAGAGATGGTCTTTGAAATTGTTAA
GTATCCGAGAATATTTCGGGGTCATACATTAATCCAGAAAGGACTAGTCTGTGTCTCCGCAGAAAAAT
TTGTTAAGTCACCAGGGAAAGTACAATCTGGAATGGACTATCTCTTCATTCCGACATTTCTGTCAGTGA
CTTACTGTCCAGCTGCAATCAAATTTCAGGTACCTGGCCCCATGTTGAAAATGAGATCAAGATACACT
CAGAGCTTACAACTTGAACTAATGATAAGAATCCTGTGTAAGCCCGATTCGCCACTTATGAAGGTCCA
TATCCCTGACAAGGAAGGAAGAGGATGTCTTGTATCAGTATGGTTGCATGTATGCAATATCTTCAAAT
CAGGAAACAAGAATGGCAGTGAGTGGCAGGAATACTGGATGAGAAAGTGTGCTAACATGCAACTTGA
AGTGTCGATTGCAGATATGTGGGGACCAACTATCATAATTCATGCCAGAGGTCACATTCCCAAAAGTG
CTAAGTTGTTTTTTGGAAAGGGTGGATGGAGCTGCCATCCACTTCACGAAGTTGTTCCAAGTGTCACTA
AAACACTATGGTCCGTGGGCTGTGAGATTACAAAGGCGAAGGCAATAATACAAGAGAGTAGCATCTC
TCTTCTCGTGGAGACTACTGACATCATAAGTCCAAAAGTCAAAATTTCATCTAAGCATCGCCGCTTTGG
GAAATCAAATTGGGGTCTGTTCAAGAAAACCAAATCACTGCCCAACCTGACGGAGCTGGAATGACTG
ACCCCCAATCGAGACTACACCACCTCAAACTATAGGTGGGTGGTACCTCAGTGATTAATCTCGTAAGC
ACTGATCGTAGGCTACAACACACTAATATTATCCAGATTAGAGAGCTTAATTAGCTCTGTATTAATAAT
AACACTACTATTCCAATAACTGGAATCACCAGCTTGATTTATCTCCAAAATGATTCAAAGAAAACAAA
TCATATTAAGACTATCCTAAGCACGAACCCATATCGTCCTTCAAATCATGGGTACTATAATTCAATTTC
TGGTGGTCTCCTGTCTATTGGCAGGAGCAGGCAGTCTTGATCCAGCAGCCCTCATGCAAATCGGTGTC
ATTCCAACAAATGTCCGGCAACTTATGTATTATACTGAGGCCTCATCAGCATTCATTGTTGTGAAGTTA
ATGCCTACAATTGACTCGCCGATTAGTGGATGTAATATAACATCAATTTCAAGCTATAATGCAACAGT
GACAAAACTCCTACAGCCGATCGGTGAGAATTTGGAGACGATTAGGAACCAGTTGATTCCAACTCGGA
GGAGACGCCGGTTTGCAGGGGTGGTGATTGGATTAGCTGCATTAGGAGTAGCTACTGCCGCACAGGTC
ACTGCCGCAGTAGCACTAGTAAAGGCAAATGAAAATGCTGCGGCTATACTCAATCTCAAAAATGCAAT
CCAAAAAACAAATGCAGCAGTTGCAGATGTGGTCCAGGCCACACAATCACTAGGAACaGCAGTTCAAG
CAGTTCAAGATCACATAAACAGTGTGGTAAGTCCAGCAATTACAGCAGCCAATTGTAAAGCCCAAGAT
GCTATCATTGGCTCAATCCTCAATCTCTATTTGACCGAGTTGACAACTATCTTCCACAATCAAATTACA
AACCCTGCATTGAGTCCTATTACAATTCAAGCTTTAAGGATCCTACTAGGGAGTACCTTGCCGACTGTG
GTCGAAAAATCTTTCAATACCCAGATAAGTGCGGCTGAGCTTCTCTCATCAGGGTTATTGACAGGCCA
GATTGTGGGATTAGATTTGACCTATATGCAGATGGTCATAAAAATTGAGCTGCCAACTTTAACTGTAC
AACCTGCAACCCAGATCATAGATCTGGCCACCATTTCTGCATTCATTAACAATCAAGAAGTCATGGCC
CAATTACCAACACGTGTTATTGTGACTGGCAGCTTGATCCAAGCCTATCCCGCATCGCAATGCACTATT
ACACCCAACACTGTGTACTGTAGGTATAATGATGCCCAAGTACTCTCAGATGATACGATGGCTTGCCT
CCAAGGTAACTTGACAAGATGCACCTTCTCTCCAGTGGTTGGGAGCTTTCTCACTCGATTCGTGCTGTT
CGATGGAATAGTTTATGCAAATTGCAGGTCGATGTTGTGCAAGTGCATGCAGCCTGCTGCTGTGATCCT
ACAGCCGAGTTCATCCCCTGTAACTGTCATTGACATGTACAAATGTGTGAGTCTGCAGCTTGACAATCT
CAGATTCACCATCACTCAATTGGCCAATGTAACCTACAATAGCACCATCAAGCTTGAAACATCCCAGA
TCTTGCCTATTGATCCGTTGGATATATCCCAGAATCTAGCTGCGGTGAATAAGAGTCTAAGTGATGCAC
TACAACACTTAGCACAAAGTGACACATACCTTTCTGCAATCACATCAGCTACGACTACAAGTGTATTA
TCCATAATAGCAATCTGTCTTGGATCGTTAGGTTTAATATTAATAATCTTGCTCAGTGTAGTTGTGTGG
AAGTTATTGACCATTGTCGCTGCTAATCGAAATAGAATGGAGAATTTTGTTTATCATAATTCAGCATTC
CACCACTCACGATCTGATCTCAGTGAGAAAAATCAACCTGCAACTCTTGGAACAAGATAAGACAGTCA
TCCATTAGTAATTTTTAAGAAAAAAACGATAGGACCGAAACTAGTATTGAAAGAACCGTCTCGGTCAA
TCTAGGTAATCGAGCTGcTACCGTCTCGGAAAGCTCAAATCATGCTGCCTGATCCGGAAGATCCGGAA
AGCAAAAAAGCTACAAGGAGAACAGGAAACCTAATTATCTGCTTCCTATTCATCTTCTTTCCGTTTGTA
AACTTCATTGTTCCAACTCTAAGACACTTGCTGTCCTAACACCTGCTATAGGCTATCCACTGCATCATC
TCTTTTTAAGAAAAAAATAGGCCCGGACGGGTTAGCAACAAGCGACTGCCGGTGCCAACAACACAAT
CCACAATCTACAGTCGACGCGGCCGCCatgttcgtcttcctggtcctgctgcctctggtctcctcacag
tgcgtcaatctgagaactcggactcagctgccacctgcttatactaatagcttcaccagaggcgtgtact
atcctgacaaggtgtttagaagctccgtgctgcactctacacaggatctgtttctgccattctttagcaa
cgtgacctggttccacgccatccacgtgagcggcaccaatggcacaaagcggttcgacaatcccgtgc
tgccttttaacgatggcgtgtacttcgcctctaccgagaagagcaacatcatcagaggctggatctttg
gcaccacactggactccaagacacagtctctgctgatcgtgaacaatgccaccaacgtggtcatcaagg
tgtgcgagttccagttttgtaatgatcccttcctgggcgtgtactatcacaagaacaataagagctgg
atggagtccggcgtgtattctagcgccaacaactgcacatttgagtacgtgagccagcctttcctgatgg
acctggagggcaagcagggcaatttcaagaacctgagggagttcgtgtttaagaatatcgacggctactt
caaaatctactctaagcacacccccatcaacctggtgcgcgacctgcctcagggcttcagcgccctgga
gcccctggtggatctgcctatcggcatcaacatcacccggtttcagacactgctggccctgcacagaa
gctacctgacacccggcgactcctctagcggatggaccgccggcgctgccgcctactatgtgggctac
ctccagccccggaccttcctgctgaagtacaacgagaatggcaccatcacagacgcagtggattgcgc
cctggaccccctgagcgagacaaagtgtacactgaagtcctttaccgtggagaagggcatctatcagac
atccaatttcagggtgcagccaaccgagtctatcgtgcgctttcctaatatcacaaacctgtgcccattt
ggcgaggtgttcaacgcaacccgcttcgccagcgtgtacgcctggaataggaagcggatcagcaactgcg
tggccgactatagcgtgctgtacaactccgcctctttcagcacctttaagtgctatggcgtgtccccca
caaagctgaatgacctgtgctttaccaacgtctacgccgattctttcgtgatcaggggcgacgaggtgc
gccagatcgcccccggccagacaggcaagatcgcagactacaattataagctgccagacgatttcacc
ggctgcgtgatcgcctggaacagcaacaatctggattccaaagtgggcggcaactacaattatagatac
cggctgtttagaaagagcaatctgaagcccttcgagagggacatctctacagaaatctaccaggccggca
gcaagccttgcaatggcgtggagggctttaactgttatttcccactccagtcctacggcttccagccca
caaacggcgtgggctatcagccttaccgcgtggtggtgctgagctttgagctgctgcacgccccagcaa
cagtgtgcggccccaagaagtccaccaatctggtgaagaacaagtgcgtgaacttcaacttcaacggcc
tgaccggcacaggcgtgctgaccgagtccaacaagaagttcctgccatttcagcagttcggcagggac
atcgcagataccacagacgccgtgcgcgacccacagaccctggagatcctggacatcacaccctgctctt
tcggcggcgtgagcgtgatcacacccggcaccaatacaagcaaccaggtggccgtgctgtatcagggc
gtgaattgtaccgaggtgcccgtggctatccacgccgatcagctgaccccaacatggcgggtgtacagc
accggctccaacgtcttccagacaagagccggatgcctgatcggagcagagcacgtgaacaattccta
tgagtgcgacatcccaatcggcgccggcatctgtgcctcttaccagacccagacaaactctagaagaa
gagcccggagcgtggcctcccagtctatcatcgcctataccatgtccctgggcgccgagaacagcgtggc
ctactctaacaatagcatcgccatcccaaccaacttcacaatctctgtgaccacagagatcctgcccgtg
tccatgaccaagacatctgtggactgcacaatgtatatctgtggcgattctaccgagtgcagcaacct
gctgctccagtacggcagcttttgtacccagctgaatagagccctgacaggcatcgccgtggagcagga
taagaacacacaggaggtgttcgcccaggtgaagcaaatctacaagaccccccctatcaaggactttggc
ggcttcaatttttcccagatcctgcctgatccatccaagccttctaagcggagctttatcgaggacctgc
tgttcaacaaggtgaccctggccgatgccggcttcatcaagcagtatggcgattgcctgggcgacatcgc
agccagggacctgatctgcgcccagaagtttaatggcctgaccgtgctgccacccctgctgacagatgag
atgatcgcacagtacacaagcgccctgctggccggcaccatcacatccggatggaccttcggcgcaggag
ccgccctccagatcccctttgccatgcagatggcctataggttcaacggcatcggcgtgacccagaatgt
gctgtacgagaaccagaagctgatcgccaatcagtttaactccgccatcggcaagatccaggacagcct
gtcctctacagccagcgccctgggcaagctccagaacgtggtgaatcagaacgcccaggccctgaatacc
ctggtgaagcagctgagcagcaacttcggcgccatctctagcgtgctgaatgacatcctgagccggctgg
acaaggtggaggcagaggtgcagatcgaccggctgatcaccggccggctccagagcctccagacctatgt
gacacagcagctgatcagggccgccgagatcagggccagcgccaatctggcagcaaccaagatgtccgag
tgcgtgctgggccagtctaagagagtggacttttgtggcaagggctatcacctgatgtccttccctcag
tctgccccacacggcgtggtgtttctgcacgtgacctacgtgcccgcccaggagaagaacttcaccacag
cccctgccatctgccacgatggcaaggcccactttccaagggagggcgtgttcgtgtccaacggcaccca
ctggtttgtgacacagcgcaatttctacgagccccagatcatcaccacagacaacaccttcgtgagcggc
aactgtgacgtggtcatcggcatcgtgaacaataccgtgtatgatccactccagcccgagctggacagct
ttaaggaggagctggataagtatttcaagaatcacacctcccctgacgtggatctgggcgacatcagcgg
catcaatgcctccgtggtgaacatccagaaggagatcgaccgcctgaacgaggtggctaagaatctgaa
cgagagcctgatcgacctccaggagctgggcaagtatgagcagtacatcaagtggccctggtacatctgg
ctgggcttcatcgccggcctgatcgccatcgtgatggtgaccatcatgctgtgctgtatgacatcctgct
gttcttgcctgaagggctgctgtagctgtggctcctgctgtAAGTTATTGACCATTGTCGTTGCTAATCG
AAATAGAATGGAGAATTTTGTTTATCATAAATGAGCTAGCCTCCTGCCATACTTCCTACTCACATCATA
TCTATTTTAAAGAAAAAATAGGCCCGAACACTAATCGTGCCGGCAGTGCCACTGCACACACAACACTA
CACATACAATACACTACAATGGTTGCAGAAGATGCCCCTGTTAGGGGCACTTGCCGAGTATTATTTCG
AACAACAACTTTAATTTTTCTATGCACACTATTAGCATTAAGCATCTCTATCCTTTATGAGAGTTTAAT
AACCCAAAAGCAAATCATGAGCCAAGCAGGCTCAACTGGATCTAATTCTAGATTAGGAAGTATCACTG
ATCTTCTTAATAATATTCTCTCTGTCGCAAATCAGATTATATATAACTCTGCAGTCGCTCTACCTCTACA
ATTGGACACTCTTGAATCAACACTCCTTACAGCCATTAAGTCTCTTCAAACAAGTGACAAGCTAGAAC
AGAACTGCTCGTGGGGTGCTGCACTGATTAATGATAATAGATACATTAATGGCATCAATCAGTTCTATT
TCTCAATTGCTGAGGGTCGCAAGCTGACACTTGGCCCACTTCTTAATATACCTAGTTTCATTCCAACTG
CCACGACACCAGAGGGCTGCACCAGGATCCCATCATTCTCGCTCACCAAGACACACTGGTGTTATACA
CACAATGTTATCCTGAATGGATGCCAGGATCATGTATCCTCAAATCAATTTGTTTCCATGGGAATCATT
GAACCCACTTCTGCCGGGTTTCCATCCTTTCGAACCCTAAAGACTCTATATCTCAGCGATGGGGTCAAT
CGTAAGAGCTGCTCTATCAGTACAGTTCCGGGGGGTTGTATGATGTACTGTTTCGTCTCTACTCAACCA
GAGAGGGATGACTACTTTTCTACCGCTCCTCCAGAACAACGAATTATTATAATGTACTATAATGATAC
AATCGTGGAGCGCATAATTAATCCACCCGGGGTACTAGATGTATGGGCAACATTGAACCCAGGAACA
GGAAGCGGGGTATATTATTTAGGTTGGGTACTCTTTCCAATATATGGCGGCGTGATTAAAGATACGAG
TTTATGGAATAATCAAGCAAATAAATACTTTATCCCCCAGATGGTTGCTGCTCTCTGCTCACAAAACCA
GGCAACTCAAGTCCAAAATGCTAAGTCATCATACTATAGCAGCTGGTTTGGCAATCGAATGATTCAGT
CTGGGATCCTGGCATGTCCTCTTCAACAGGATCTAACCAATGAGTGTTTAGTTCTGCCCTTTTCTAATG
ATCAGGTGCTTATGGGTGCTGAAGGGAGATTATACATGTATGGTGACTCGGTGTATTACTACCAAAGA
AGCAATAGTTGGTGGCCTATGACCATGCTGTATAAGGTAACCATAACATTCACTAATGGTCAGCCATC
TGCTATATCAGCTCAGAATGTGCCCACACAGCAGGTCCCTAGACCTGGGACAGGAAGCTGCTCTGCAA
CAAATAGATGTCCCGGTTTTTGCTTGAAAGGAGTGTATGCTGATGCCTGGTTACTGACCAACCCTTCGT
CTACCAGTACATTTGGATCAGAAGCAACCTTCACTGGTTCTTATCTCAACGCAGCAACTCAGCGTATCA
ATCCGACGATGTATATCGCGAACAACACACAGATCATAAGCTCACAGCAATTTGGATCAAGCGGTCAA
GAAGCAGCATATGGCCACACAACTTGTTTTAGGGACACAGGCTCTGTTATGGTATACTGTATCTATATT
ATTGAATTGTCCTCATCTCTCTTAGGACAATTTCAGATTGTCCCATTTATCCGTCAGGTGACACTATCCT
AAAGGCAGAAGCCTCCAGGTCTGACCCAGCCAATCAAAGCATTATACCAGACCATGGAATGCATACC
AAACATTATTGACACTAATGACACACAAAATTGGTTTTAAGAAAAACCAAGAGAACAATAGGCCAGA
ATGGCTGGGTCTCGGGAGATATTACTCCCTGAAGTCCATCTCAATTCACCAATTGTAAAGCATAAGCT
ATACTATTACATTCTACTTGGAAACCTCCCAAATGAGATCGACATTGACGATTTAGGTCCATTACATAA
TCAAAATTGGAATCAAATAGCACATGAAGAGTCTAACTTAGCCCAACGCTTGGTAAATGTAAGAAATT
TTCTAATTACCCACATCCCTGATCTTAGAAAGGGCCATTGGCAAGAGTATGTCAATGTAATACTGTGGC
CGCGAATTCTTCCCTTGATCCCGGATTTTAAAATCAATGACCAATTGCCTCTACTCAAAAATTGGGACA
AGTTAGTTAAAGAATCATGTTCAGTAATCAATGCGGGTACTTCCCAGTGCATTCAGAATCTCAGCTATG
GACTGACAGGTCGTGGGAACCTCTTTACACGATCACGTGAACTCTCTGGTGACCGCAGGGATATTGAT
CTTAAGACGGTTGTGGCAGCATGGCATGACTCAGACTGGAAAAGAATAAGTGATTTTTGGATTATGAT
CAAATTCCAGATGAGACAATTAATTGTTAGGCAAACAGATCATAATGATCCTGATTTAATCACGTATA
TCGAAAATAGAGAAGGCATAATCATCATAACCCCTGAACTGGTAGCATTATTTAACACTGAGAATCAT
ACACTAACATACATGACCTTTGAAATTGTACTGATGGTTTCAGATATGTACGAAGGTCGTCACAACATT
TTATCACTATGCACAGTTAGCACTTACCTGAATCCTCTGAAGAAAAGAATAACATATTTATTGAGCCTT
GTAGATAACTTAGCTTTTCAGATAGGTGATGCTGTATATAACATAATTGCTTTGCTAGAATCCTTTGTA
TATGCACAGTTGCAAATGTCAGATCCCATCCCAGAACTCAGAGGACAATTCCATGCATTCGTATGTTCT
GAGATTCTTGATGCACTAAGGGGAACTAATAGTTTCACCCAGGATGAATCAAGAACTGTGACAACCAA
TTTGATATCCCCATTCCAAGATCTGACCCCAGATCTTACGGCTGAATTGCTCTGTATAATGAGGCTTTG
GGGACACCCCATGCTCACCGCCAGTCAAGCTGCGGGAAAGGTACGCGAGTCCATGTGTGCTGGAAAA
GTATTAGACTTTCCCACCATTATGAAAACACTAGCCTTTTTCCATACTATTCTGATCAATGGATACAGG
AGGAAGCATCATGGAGTATGGCCACCCTTAAACTTACCGGGTAATGCTTCAAAGGGTCTCACGGAACT
TATGAATGACAATACTGAGATAAGCTATGAATTCACACTTAAGCATTGGAAGGAAATCTCTCTTATAA
AATTCAAGAAATGTTTTGATGCAGACGCAGGTGAGGAACTCAGTATATTTATGAAAGATAAAGCAATT
AGTGCCCCAAAACAAGATTGGATGAGTGTGTTTAGAAGAAGCCTAATCAAACAGCGCCATCAGCATC
ATCAGGTCCCCCTACCAAATCCATTCAATCGACGGCTATTGCTAAACTTTCTCGGAGATGACAAATTCG
ACCCGAATGTGGAGCTACAGTATGTAACATCAGGTGAGTATCTACATGATGACACGTTTTGTGCATCA
TATTCACTAAAAGAGAAGGAAATTAAACCTGATGGTCGAATTTTTGCAAAGTTGACTAAGAGAATGAG
ATCATGTCAAGTTATAGCAGAATCTCTTTTAGCGAACCATGCTGGGAAGTTAATGAAAGAGAATGGTG
TTGTGATGAATCAGCTATCATTAACAAAATCACTATTAACAATGAGTCAGATTGGAATAATATCCGAG
AAAGCTAGAAAATCGACTCGAGATAACATAAATCAACCTGGTTTCCAGAATATCCAGAGAAATAAAT
CACATCACTCCAAGCAAGTCAATCAGCGAGATCCAAGTGATGACTTTGAATTGGCAGCATCTTTTTTA
ACTACTGATCTCAAAAAATATTGTTTACAATGGAGGTACCAGACAATTATCCCATTTGCTCAATCATTA
AACAGAATGTATGGTTATCCTCATCTCTTTGAGTGGATTCACTTACGGCTAATGCGTAGTACACTTTAC
GTGGGGGATCCCTTCAACCCACCAGCAGATACCAGTCAATTTGATCTAGATAAAGTAATTAATGGAGA
TATCTTCATTGTATCACCCAGAGGTGGAATTGAAGGGCTATGTCAAAAGGCTTGGACAATGATATCTA
TCTCTGTGATAATTCTATCTGCCACAGAGTCTGGCACACGAGTAATGAGTATGGTGCAGGGAGATAAT
CAAGCAATTGCTGTCACCACACGAGTACCAAGGAGCCTGCCGACTCTTGAGAAAAAGACTATTGCTTT
TAGATCTTGTAATCTATTCTTTGAGAGGTTAAAATGTAATAATTTTGGATTAGGTCACCATTTGAAAGA
ACAAGAGACTATCATTAGTTCTCACTTCTTTGTTTATAGCAAGAGAATATTCTATCAGGGGAGGATTCT
AACGCAAGCCTTAAAAAATGCTAGTAAGCTCTGCTTGACAGCTGATGTCCTAGGAGAATGTACCCAAT
CATCATGTTCTAATCTTGCAACTACTGTCATGAGGTTAACTGAGAATGGTGTTGAAAAAGATATCTGTT
TCTACTTGAATATCTATATGACCATCAAACAGCTCTCCTATGATATCATCTTCCCTCAAGTGTCAATTCC
TGGAGATCAGATCACATTAGAATACATAAATAATCCACACCTGGTATCACGATTGGCTCTTCTGCCATC
CCAGCTAGGAGGTCTAAACTACCTGTCATGCAGTAGGCTGTTCAATCGAAACATAGGCGACCCGGTGG
TTTCCGCAGTTGCAGATCTTAAGAGATTAATTAAATCAGGATGTATGGATTACTGGATCCTTTATAACT
TATTAGGGAGAAAACCGGGAAACGGCTCATGGGCTACTTTAGCAGCTGACCCGTACTCAATCAATATA
GAGTATCAATACCCCCCAACTACAGCTCTTAAGAGGCACACCCAACAAGCTCTGATGGAACTCAGTAC
GAATCCAATGTTACGTGGCATATTCTCTGACAATGCACAGGCAGAAGAAAATAATCTTGCTAGATTTC
TCCTGGATAGGGAGGTGATCTTTCCGCGTGTAGCTCACATCATCATTGAGCAAACCAGTGTCGGGAGG
AGAAAACAGATTCAAGGATATTTGGATTCAACTAGATCGATAATGAGTAAATCACTAGAAATTAAGCC
CTTGTCCAATAGGAAGCTTAATGAAATACTGGATTACAACATCAATTACCTAGCTTACAATTTGGCATT
ACTCAAGAATGCTATTGAACCTCCGACTTATTTGAAAGCAATGACTCTTGAAACATGTAGCATCGACA
TTGCAAGGAGCCTCCGGAAGCTCTCCTGGGCCCCACTCTTGGGTGGGAGAAATCTTGAAGGATTAGAG
ACGCCAGATCCCATTGAAATTACTGCAGGAGCATTAATTGTTGGATCGGGCTACTGTGAACAGTGTGC
TGCAGGAGACAATCGATTCACATGGTTTTTCTTGCCATCTGGTATCGAGATAGGAGGGGATCCCCGTG
ATAATCCTCCTATCCGTGTACCGTACATTGGCTCCAGGACTGATGAGAGGAGGGTAGCCTCAATGGCA
TACATCAGGGGTGCCTCGAGTAGCCTAAAAGCAGTTCTTAGACTGGCGGGAGTGTACATCTGGGCATT
CGGAGATACTCTGGAGAATTGGATAGATGCACTGGATTTGTCTCACACTAGAGTTAACATCACACTTG
AACAGCTGCAATCCCTCACCCCACTTCCAACCTCTGCCAATCTAACCCATCGGTTGGATGATGGCACA
ACTACCCTAAAGTTTACTCCTGCGAGCTCTTACACCTTTTCAAGTTTCACTCATATATCAAATGATGAG
CAATACCTGACAATTAATGACAAAACTGCAGATTCAAATATAATCTACCAACAGTTAATGATCACTGG
ACTCGGAATTTTAGAAACATGGAATAATCCCCCAATCAATAGAACATTCGAAGAATCTACCCTACATT
TGCACACTGGTGCATCATGTTGTGTCCGACCTGTGGACTCCTGCATCATCTCAGAAGCATTAACAGTCA
AGCCACATATTACAGTACCGTACAGCAATAAATTTGTATTTGATGAAGACCCGCTATCTGAATATGAG
ACTGCAAAACTGGAATCGTTATCATTTCAAGCCCAATTAGGCAACATTGATGCTGTAGATATGACAGG
TAAATTAACATTATTGTCCCAATTCACTGCAAGGCAGATTATTAATGCAATCACTGGACTCGATGAGTC
TGTCTCTCTTACTAATGATGCCATTGTTGCATCAGACTATGTCTCCAATTGGATTAGTGAATGCATGTA
TACCAAATTAGATGAATTATTTATGTATTGTGGGTGGGAACTACTATTGGAACTATCCTATCAAATGTA
TTATCTGAGGGTAGTTGGGTGGAGTAATATAGTGGATTATTCTTACATGATCTTGAGAAGAATCCCGG
GTGCAGCATTAAACAATCTGGCATCTACATTAAGTCATCCAAAACTTTTCCGACGAGCTATCAACCTA
GATATAGTTGCCCCCTTAAATGCTCCTCATTTTGCATCTCTGGACTACATCAAGATGAGTGTGGATGCA
ATACTCTGGGGCTGTAAAAGAGTCATCAATGTGCTCTCCAATGGAGGGGACTTAGAATTAGTTGTGAC
ATCTGAAGATAGCCTTATTCTCAGTGACCGATCCATGAATCTCATTGCAAGGAAATTAACTTTATTATC
ACTGATTCACCATAATGGTTTGGAACTACCAAAGATTAAGGGGTTCTCTCCTGATGAGAAGTGTTTCGC
TTTGACAGAATTTTTGAGGAAAGTGGTGAACTCAGGGTTGAGTTCAATAGAGAACCTATCAAATTTTA
TGTACAATGTGGAGAACCCACGGCTTGCAGCATTCGCCAGCAACAATTACTACCTGACCAGAAAATTA
TTGAATTCAATACGAGATACTGAGTCGGGTCAAGTAGCAGTCACCTCATATTATGAATCATTAGAATA
TATTGATAGTCTTAAGCTAACCCCACATGTGCCTGGTACCTCATGCATTGAGGATGATAGTCTATGTAC
AAATGATTACATAATCTGGATCATAGAGTCTAATGCAAACTTGGAGAAGTATCCAATTCCAAATAGCC
CTGAGGATGATTCCAATTTCCATAACTTTAAGTTGAATGCTCCATCGCACCATACCTTACGCCCATTAG
GGTTGTCATCAACTGCTTGGTATAAGGGTATAAGCTGTTGCAGGTACCTTGAGCGATTAAAGCTACCA
CAAGGTGATCATTTATATATTGCAGAAGGTAGTGGTGCCAGTATGACAATCATAGAATACCTATTCCC
AGGAAGAAAGATATATTACAATTCTTTATTTAGTAGTGGTGACAATCCCCCACAAAGAAATTATGCAC
CAATGCCTACTCAGTTCATTGAGAGTGTCCCATACAAGCTCTGGCAAGCACACACAGATCAATATCCC
GAGATTTTTGAGGACTTCATCCCTCTATGGAACGGAAATGCCGCCATGACTGACATAGGAATGACAGC
TTGTGTAGAATTCATCATCAATCGAGTCGGCCCAAGGACTTGCAGTTTAGTACATGTAGATTTGGAATC
AAGTGCAAGCTTAAATCAACAATGCCTGTCAAAGCCGATAATTAATGCTATCATCACTGCTACAACTG
TTTTGTGCCCTCATGGGGTGCTTATTCTGAAATATAGTTGGTTGCCATTTACTAGATTTAGTACTTTGAT
CACTTTCTTATGGTGCTACTTTGAGAGAATCACTGTTCTTAGGAGCACATATTCTGGTCCAGCTAATCA
TGAGGTTTATTTAATTTGTATCCTTGCCAACAACTTTGCATTCCAGACTGTCTCGCAGGCAACAGGAAT
GGCGATGACTTTAACCGATCAAGGGTTTACTTTGATATCACCTGAAAGAATAAATCAGTATTGGGATG
GTCACTTGAAGCAAGAACGTATCGTAGCAGAAGCAATTGATAAGGTGGTTCTAGGAGAAGATGCTCTA
TTCAATTCGAGTGATAATGAATTAATTCTCAAATGTGGAGGGACACCAAATGCACGGAATCTTATCGA
TATCGAGCCAGTCGCAACTTTCATAGAATTTGAACAACTGATCTGCACAATGTTGACAACCCACTTGA
AGGAAATAATTGATATAACAAGGTCTGGAACCCAGGATTATGAAAGTTTATTACTCACTCCTTACAAT
TTAGGTCTTCTTGGTAAAATCAGTACGATAGTGAGATTATTAACAGAAAGGATTCTAAATCATACTATC
AGGAATTGGTTGATCCTCCCACCTTCGCTCCGGATGATCGTGAAGCAGGACTTGGAATTCGGCATATTC
AGGATTACTTCCATCCTCAATTCTGATCGGTTCCTGAAGCTTTCTCCAAATAGGAAATACTTGATTACA
CAATTAACTGCAGGCTACATTAGGAAATTGATTGAGGGGGATTGTAATATCGATCTAACCAGACCTAT
CCAAAAACAAATCTGGAAAGCATTAGGTTGTGTAGTCTATTGTCACGATCCAGTAGATCAAAGGGAAT
CAACAGAGTTTATTGATATAAATATTAATGAAGAAATAGACCGCGGGATCGATGGCGAGGAAATCTA
AATATATCAAGAATCAGAATTAGTTTAAGAAAAAAGAAGAGGATTAATCTTGGTTTTCCCCTTGGT.

13. CVL64

The nucleic acid sequence for CVL64 is:

(SEQ ID NO: 73)
ACCAGGGGGAAAACGAAGTGGTGACTCAAATCATCGAAGACCCTCGAGATTACATAGGTCCGGAACC
TATGGCCTTCGTGACCGACCTCGAGTCAGAGTAGTTCAATAAGGACCTATCAAGTTTGGGCAATTTTTC
GTCCCCGACACAAAAATGTCATCCGTGCTTAAAGCATATGAGCGATTCACACTCACTCAAGAACTGCA
AGATCAGAGTGAGGAAGGTACAATCCCACCTACAACACTAAAACCGGTAATCAGGGTATTTATACTAA
CCTCTAATAACCCAGAGCTAAGATCCCGGCTTCTTCTATTCTGCCTACGGATTGTTCTCAGTAATGGTG
CAAGGGATTCCCATCGCTTTGGAGCATTACTTACAATGTTTTCGCTACCATCAGCCACAATGCTCAATC
ATGTCAAATTAGCTGACCAGTCACCAGAAGCTGATATCGAAAGGGTAGAGATCGATGGCTTTGAGGA
GGGATCATTCCGCTTAATCCCCAATGCTCGTTCAGGTATGAGCCGTGGAGAGATCAATGCCTATGCTG
CACTTGCAGAAGATCTACCTGACACACTAAACCATGCAACACCTTTTGTTGATTCCGAAGTCGAGGGA
ACTGCATGGGATGAGATTGAGACTTTCTTAGATATGTGTTACAGTGTCCTAATGCAGGCATGGATAGT
GACTTGCAAGTGCATGACTGCGCCAGACCAACCTGCTGCTTCTATTGAGAAACGCCTGCAAAAATATC
GTCAGCAAGGCAGGATCAACCCGAGATATCTCCTGCAACCGGAGGCTCGAAGAATAATCCAGAATGT
AATCCGAAAGGGAATGGTGGTCAGACATTTCCTCACCTTTGAACTGCAGCTTGCCCGAGCACAAAGCC
TTGTATCAAATAGGTATTATGCTATGGTAGGGGATGTTGGAAAGTATATAGAGAATTGTGGAATGGGA
GGCTTCTTTTTGACACTAAAATATGCATTAGGAACCAGATGGCCCACACTTGCTTTAGCTGCATTTTCA
GGAGAGCTAACAAAGCTAAAGTCCCTCATGGCATTATACCAGACCCTTGGTGAGCAGGCCCGATATTT
GGCCCTATTGGAGTCACCACATTTGATGGATTTTGCTGCAGCAAACTACCCACTGCTATATAGCTATGC
TATGGGAATAGGCTATGTGTTAGATGTCAACATGAGGAACTACGCTTTCTCCAGATCATACATGAATA
AGACATATTTCCAATTGGGAATGGAAACTGCAAGAAAACAACAGGGTGCAGTTGACATGAGGATGGC
AGAAGATCTCGGTCTAACTCAAGCCGAACGCACCGAGATGGCAAATACACTTGCCAAATTGACCACA
GCAAATCGAGGGGCAGACACCAGGGGAGGAGTCAACCCGTTCTCATCTATCACTGGGACAACTCAGG
TGCCCGCTGCAGCAACAGGTGACACATTCGAGAGTTACATGGCAGCGGATCGACTGAGGCAGAGATA
TGCTGATGCAGGCACCCACGATGATGAGATGCCACCATTGGAAGAGGAGGAAGAGGACGACACATCT
GCAGGTCCACGCACTGGACTAACTCTTGAACAAGTGGCCTTGGACATCCAGAACGCAGCAGTTGGAGC
TCCCATCCATACAGATGACCTGAATGCCGCACTGGGTGATCTTGACATCTAGACAATTCAGATCCCAA
TCTTAAATCGACACACCTAATTGACCAGTTAGATGGAACTACAGTGGATTCCATAAGGTTCCTGCCTAC
CATCGGCTTTTAAGAAAAAAATAGGCCCGGACGGGTTAGCAACAAGCGACTGCCGATGCCAATAACA
CAATCCACAATCTACAATGGATCCCACTGATCTGAGCTTCTCCCCAGATGAGATCAATAAGCTCATAG
AGACAGGCCTGAATACTGTGGAGTATTTTACTTCCCAACAAGTCACAGGAACATCCTCTCTTGGAAAG
AATACAATACCACCAGGGGTCACAGGACTACTAACCAATGCTGCAGAGGCAAAGATCCAAGAGTCAA
CCAACCATCAGAAGGGTTCAGTTGGTGGGGGCGCAAAACCAAAGAAACCGCGACCAAAAATTGCCAT
TGTGCCAGCAGATGACAAAACGGTGCCCGGAAAGCCGATCCCAAACCCTCTATTAGGTCTGGACTCCA
CCCCGAGCACCCAAACTGTGCTTGATCTAAGTGGGAAAACATTACCATCAGGATCCTATAAGGGGGTT
AAACTTGCGAAATTTGGAAAAGAAAATCTGATGACACGGTTCATCGAGGAACCCAGAGAGAATCCTA
TCGCAACCAATTCCCCCATCGATTTTAAGAGGGGCAGGGATACCGGCGGGTTCCATAGAAGGGAGTAC
TCAATCGGATGGGTGGGAGATGAAGTCAAGGTCACTGAGTGGTGCAATCCATCCTGTTCTCCAATCAC
CGCTGCAGCAAGGCGATTTGAATGCACTTGTCACCAGTGTCCAGTCACTTGCTCTGAATGTGAACGAG
ATACTTAATACAGTGAGAAATTTGGACTCTCGGATGAATCAACTGGAGACAAAAGTAGATCGCATTCT
CTCATCTCAGTCTCTAATCCAGACCATCAAGAATGACATAGTTGGACTTAAAGCAGGGATGGCTACTT
TAGAAGGAATGATTACAACTGTGAAAATCATGGACCCGGGAGTTCCCAGTAATGTTACTGTGGAAGAT
GTACGCAAGAAACTAAGTAACCATGCTGTTGTTGTGCCAGAATCATTCAATGATAGTTTCTTGACTCAA
TCTGAAGATGTAATTTCACTTGATGAGTTGGCTCGACCAACTGCAACAAGTGTTAAGAAGATTGTCAG
GAAGGTTCCTCCTCAGAAGGATCTGACTGGATTGAAGATCACACTAGAGCAATTGGCAAAGGATTGCA
TCAGCAAACCGAAGATGAGGGAAGAGTATCTCCTCAAAATCAACCAGGCTTCCAGTGAGGCTCAGCT
AATTGACCTCAAGAAAGCAATCATCCGCAGTGCAATTTGATCAAGAAACACCCAATTACACTACACTG
GTATGACACTGTACTAACCCTGAGGGTTTTAGAAAAAACGATTAACGATAAATAAGCCCGAACACTAC
ACACCACCCGAGGCAGCCATGCCATCCATCAGCATCCCCGCAGACCCCACCAATCCACGTCAATCAAT
AAAAGCGTTCCCAATTGTGATCAACCGTGATGGGGGTGAGAAAGGTCGCTTGGTTAAACAACTACGCA
CAACCTACTTGAATGACCTAGATACTCATGAGCCACTGGTGACATTCGTAAATACTTATGGATTCATCT
ACGAACAGGATCGGGGAAATACCATTGTCGGAGAGGATCAACATGGGAAGAAAAGAGAGGCTGTGA
CTGCTGCAATGGTTACCCTTGGATGTGGGCCTAATCTACCATCATTAGGGAATGTCCTGGGACAACTG
AGTGAATTCCAGGTCATTGTTAGGAAGACATCCAGCAAAGCGGAAGAGATGGTCTTTGAAATTGTTAA
GTATCCGAGAATATTTCGGGGTCATACATTAATCCAGAAAGGACTAGTCTGTGTCTCCGCAGAAAAAT
TTGTTAAGTCACCAGGGAAAGTACAATCTGGAATGGACTATCTCTTCATTCCGACATTTCTGTCAGTGA
CTTACTGTCCAGCTGCAATCAAATTTCAGGTACCTGGCCCCATGTTGAAAATGAGATCAAGATACACT
CAGAGCTTACAACTTGAACTAATGATAAGAATCCTGTGTAAGCCCGATTCGCCACTTATGAAGGTCCA
TATCCCTGACAAGGAAGGAAGAGGATGTCTTGTATCAGTATGGTTGCATGTATGCAATATCTTCAAAT
CAGGAAACAAGAATGGCAGTGAGTGGCAGGAATACTGGATGAGAAAGTGTGCTAACATGCAACTTGA
AGTGTCGATTGCAGATATGTGGGGACCAACTATCATAATTCATGCCAGAGGTCACATTCCCAAAAGTG
CTAAGTTGTTTTTTGGAAAGGGTGGATGGAGCTGCCATCCACTTCACGAAGTTGTTCCAAGTGTCACTA
AAACACTATGGTCCGTGGGCTGTGAGATTACAAAGGCGAAGGCAATAATACAAGAGAGTAGCATCTC
TCTTCTCGTGGAGACTACTGACATCATAAGTCCAAAAGTCAAAATTTCATCTAAGCATCGCCGCTTTGG
GAAATCAAATTGGGGTCTGTTCAAGAAAACCAAATCACTGCCCAACCTGACGGAGCTGGAATGACTG
ACCCCCAATCGAGACTACACCACCTCAAACTATAGGTGGGTGGTACCTCAGTGATTAATCTCGTAAGC
ACTGATCGTAGGCTACAACACACTAATATTATCCAGATTAGAGAGCTTAATTAGCTCTGTATTAATAAT
AACACTACTATTCCAATAACTGGAATCACCAGCTTGATTTATCTCCAAAATGATTCAAAGAAAACAAA
TCATATTAAGACTATCCTAAGCACGAACCCATATCGTCCTTCAAATCATGGGTACTATAATTCAATTTC
TGGTGGTCTCCTGTCTATTGGCAGGAGCAGGCAGTCTTGATCCAGCAGCCCTCATGCAAATCGGTGTC
ATTCCAACAAATGTCCGGCAACTTATGTATTATACTGAGGCCTCATCAGCATTCATTGTTGTGAAGTTA
ATGCCTACAATTGACTCGCCGATTAGTGGATGTAATATAACATCAATTTCAAGCTATAATGCAACAGT
GACAAAACTCCTACAGCCGATCGGTGAGAATTTGGAGACGATTAGGAACCAGTTGATTCCAACTCGGA
GGAGACGCCGGTTTGCAGGGGTGGTGATTGGATTAGCTGCATTAGGAGTAGCTACTGCCGCACAGGTC
ACTGCCGCAGTAGCACTAGTAAAGGCAAATGAAAATGCTGCGGCTATACTCAATCTCAAAAATGCAAT
CCAAAAAACAAATGCAGCAGTTGCAGATGTGGTCCAGGCCACACAATCACTAGGAACaGCAGTTCAAG
CAGTTCAAGATCACATAAACAGTGTGGTAAGTCCAGCAATTACAGCAGCCAATTGTAAAGCCCAAGAT
GCTATCATTGGCTCAATCCTCAATCTCTATTTGACCGAGTTGACAACTATCTTCCACAATCAAATTACA
AACCCTGCATTGAGTCCTATTACAATTCAAGCTTTAAGGATCCTACTAGGGAGTACCTTGCCGACTGTG
GTCGAAAAATCTTTCAATACCCAGATAAGTGCGGCTGAGCTTCTCTCATCAGGGTTATTGACAGGCCA
GATTGTGGGATTAGATTTGACCTATATGCAGATGGTCATAAAAATTGAGCTGCCAACTTTAACTGTAC
AACCTGCAACCCAGATCATAGATCTGGCCACCATTTCTGCATTCATTAACAATCAAGAAGTCATGGCC
CAATTACCAACACGTGTTATTGTGACTGGCAGCTTGATCCAAGCCTATCCCGCATCGCAATGCACTATT
ACACCCAACACTGTGTACTGTAGGTATAATGATGCCCAAGTACTCTCAGATGATACGATGGCTTGCCT
CCAAGGTAACTTGACAAGATGCACCTTCTCTCCAGTGGTTGGGAGCTTTCTCACTCGATTCGTGCTGTT
CGATGGAATAGTTTATGCAAATTGCAGGTCGATGTTGTGCAAGTGCATGCAGCCTGCTGCTGTGATCCT
ACAGCCGAGTTCATCCCCTGTAACTGTCATTGACATGTACAAATGTGTGAGTCTGCAGCTTGACAATCT
CAGATTCACCATCACTCAATTGGCCAATGTAACCTACAATAGCACCATCAAGCTTGAAACATCCCAGA
TCTTGCCTATTGATCCGTTGGATATATCCCAGAATCTAGCTGCGGTGAATAAGAGTCTAAGTGATGCAC
TACAACACTTAGCACAAAGTGACACATACCTTTCTGCAATCACATCAGCTACGACTACAAGTGTATTA
TCCATAATAGCAATCTGTCTTGGATCGTTAGGTTTAATATTAATAATCTTGCTCAGTGTAGTTGTGTGG
AAGTTATTGACCATTGTCGCTGCTAATCGAAATAGAATGGAGAATTTTGTTTATCATAATTCAGCATTC
CACCACTCACGATCTGATCTCAGTGAGAAAAATCAACCTGCAACTCTTGGAACAAGATAAGACAGTCA
TCCATTAGTAATTTTTAAGAAAAAAACGATAGGACCGAAACTAGTATTGAAAGAACCGTCTCGGTCAA
TCTAGGTAATCGAGCTGcTACCGTCTCGGAAAGCTCAAATCATGCTGCCTGATCCGGAAGATCCGGAA
AGCAAAAAAGCTACAAGGAGAACAGGAAACCTAATTATCTGCTTCCTATTCATCTTCTTTCCGTTTGTA
AACTTCATTGTTCCAACTCTAAGACACTTGCTGTCCTAACACCTGCTATAGGCTATCCACTGCATCATC
TCTTTTTAAGAAAAAAATAGGCCCGGACGGGTTAGCAACAAGCGACTGCCGGTGCCAACAACACAAT
CCACAATCTACAGTCGACGCGGCCGCCatgttcgtcttcctggtcctgctgcctctggtctcctcacag
tgcgtcaatctgacaactcggactcagctgccacctgcttatactaatagcttcaccagaggcgtgtact
atcctgacaaggtgtttagaagctccgtgctgcactctacacaggatctgtttctgccattctttagcaa
cgtgacctggttccacgccatccacgtgagcggcaccaatgtgataaagcggttcgacaatcccgtgc
tgccttttaacgatggcgtgtacttcgcctctaccgagaagagcaacatcatcagaggctggatctt
tggcaccacactggactccaagacacagtctctgctgatcgtgaacaatgccaccaacgtggtcatc
aaggtgtgcgagttccagttttgtaatgatcccttcctgggcgtgtactatcacaagaacaataagagc
tggatggagtccgagtttagagtgtattctagcgccaacaactgcacatttgagtacgtgagccagcc
tttcctgatggacctggagggcaagcagggcaatttcaagaacctgagggagttcgtgtttaagaatat
cgacggctacttcaaaatctactctaagcacacccccatcaacctggtgcgcgacctgcctcagggctt
cagcgccctggagcccctggtggatctgcctatcggcatcaacatcacccggtttcagacactgctggcc
ctgcacgactcctctagcggatggaccgccggcgctgccgcctactatgtgggctacctccagccccg
gaccttcctgctgaagtacaacgagaatggcaccatcacagacgcagtggattgcgccctggaccccctg
agcgagacaaagtgtacactgaagtcctttaccgtggagaagggcatctatcagacatccaatttcaggg
tgcagccaaccgagtctatcgtgcgctttcctaatatcacaaacctgtgcccatttggcgaggtgttcaa
cgcaacccgcttcgccagcgtgtacgcctggaataggaagcggatcagcaactgcgtggccgactatag
cgtgctgtacaactccgcctctttcagcacctttaagtgctatggcgtgtcccccacaaagctgaatg
acctgtgctttaccaacgtctacgccgattctttcgtgatcaggggcgacgaggtgcgccagatcgccc
ccggccagacaggcaagatcgcagactacaattataagctgccagacgatttcaccggctgcgtgatcg
cctggaacagcaacaatctggattccaaagtgggcggcaactacaattatCAGtaccggctgtttag
aaagagcaatctgaagcccttcgagagggacatctctacagaaatctaccaggccggcagcacccctt
gcaatggcgtggagggctttaactgttattCcccactccagtcctacggcttccagcccacaaacggc
gtgggctatcagccttaccgcgtggtggtgctgagctttgagctgctgcacgccccagcaacagtgtgc
ggccccaagaagtccaccaatctggtgaagaacaagtgcgtgaacttcaacttcaacggcctgaccggca
caggcgtgctgaccgagtccaacaagaagttcctgccatttcagcagttcggcagggacatcgcagatac
cacagacgccgtgcgcgacccacagaccctggagatcctggacatcacaccctgctctttcggcggcgt
gagcgtgatcacacccggcaccaatacaagcaaccaggtggccgtgctgtatcaggGcgtgaattgtacc
gaggtgcccgtggctatccacgccgatcagctgaccccaacatggcgggtgtacagcaccggctccaac
gtcttccagacaagagccggatgcctgatcggagcagagcacgtgaacaattcctatgagtgcgacat
cccaatcggcgccggcatctgtgcctcttaccagacccagacaaactctcccagaagagcccggagcg
tggcctcccagtctatcatcgcctataccatgtccctgggcgccgagaacagcgtggcctactctaac
aatagcatcgccatcccaaccaacttcacaatctctgtgaccacagagatcctgcccgtgtccatga
ccaagacatctgtggactgcacaatgtatatctgtggcgattctaccgagtgcagcaacctgctgctcc
agtacggcagcttttgtacccagctgaatagagccctgacaggcatcgccgtggagcaggataagaac
acacaggaggtgttcgcccaggtgaagcaaatctacaagaccccccctatcaaggactttggcggcttca
atttttcccagatcctgcctgatccatccaagccttctaagcggagctttatcgaggacctgctgttcaa
caaggtgaccctggccgatgccggcttcatcaagcagtatggcgattgcctgggcgacatcgcagccagg
gacctgatctgcgcccagaagtttaatggcctgaAcgtgctgccacccctgctgacagatgagatgatcg
cacagtacacaagcgccctgctggccggcaccatcacatccggatggaccttcggcgcaggagccgccct
ccagatcccctttgccatgcagatggcctataggttcaacggcatcggcgtgacccagaatgtgctgtac
gagaaccagaagctgatcgccaatcagtttaactccgccatcggcaagatccaggacagcctgtcctct
acagccagcgccctgggcaagctccaggatgtggtgaatcagaacgcccaggccctgaataccctggtga
agcagctgagcagcaacttcggcgccatctctagcgtgctgaatgacatcctgagccggctggacaaggt
ggaggcagaggtgcagatcgaccggctgatcaccggccggctccagagcctccagacctatgtgacacag
cagctgatcagggccgccgagatcagggccagcgccaatctggcagcaaccaagatgtccgagtgcgtgc
tgggccagtctaagagagtggacttttgtggcaagggctatcacctgatgtccttccctcagtctgcccc
acacggcgtggtgtttctgcacgtgacctacgtgcccgcccaggagaagaacttcaccacagcccctgc
catctgccacgatggcaaggcccactttccaagggagggcgtgttcgtgtccaacggcacccactggttt
gtgacacagcgcaatttctacgagccccagatcatcaccacagacaacaccttcgtgagcggcaactgtg
acgtggtcatcggcatcgtgaacaataccgtgtatgatccactccagcccgagctggacagctttaagg
aggagctggataagtatttcaagaatcacacctcccctgacgtggatctgggcgacatcagcggcatcaa
tgcctccgtggtgaacatccagaaggagatcgaccgcctgaacgaggtggctaagaatctgaacgagagc
ctgatcgacctccaggagctgggcaagtatgagcagtacatcaagtggccctggtacatctggctgggct
tcatcgccggcctgatcgccatcgtgatggtgaccatcatgctgtgctgtatgacatcctgctgttcttg
cctgaagggctgctgtagctgtggctcctgctgtAAGTTATTGACCATTGTCGTTGCTAATCGAAATAGA
ATGGAGAATTTTGTTTATCATAAATGAtgaACGCGTGCTAGCCTCCTGCCATACTTCCTACTCACATCAT
ATCTATTTTAAAGAAAAAATAGGCCCGAACACTAATCGTGCCGGCAGTGCCACTGCACACACAACACT
ACACATACAATACACTACAATGGTTGCAGAAGATGCCCCTGTTAGGGGCACTTGCCGAGTATTATTTC
GAACAACAACTTTAATTTTTCTATGCACACTATTAGCATTAAGCATCTCTATCCTTTATGAGAGTTTAA
TAACCCAAAAGCAAATCATGAGCCAAGCAGGCTCAACTGGATCTAATTCTAGATTAGGAAGTATCACT
GATCTTCTTAATAATATTCTCTCTGTCGCAAATCAGATTATATATAACTCTGCAGTCGCTCTACCTCTAC
AATTGGACACTCTTGAATCAACACTCCTTACAGCCATTAAGTCTCTTCAAACAAGTGACAAGCTAGAA
CAGAACTGCTCGTGGGGTGCTGCACTGATTAATGATAATAGATACATTAATGGCATCAATCAGTTCTA
TTTCTCAATTGCTGAGGGTCGCAAGCTGACACTTGGCCCACTTCTTAATATACCTAGTTTCATTCCAAC
TGCCACGACACCAGAGGGCTGCACCAGGATCCCATCATTCTCGCTCACCAAGACACACTGGTGTTATA
CACACAATGTTATCCTGAATGGATGCCAGGATCATGTATCCTCAAATCAATTTGTTTCCATGGGAATCA
TTGAACCCACTTCTGCCGGGTTTCCATCCTTTCGAACCCTAAAGACTCTATATCTCAGCGATGGGGTCA
ATCGTAAGAGCTGCTCTATCAGTACAGTTCCGGGGGGTTGTATGATGTACTGTTTCGTCTCTACTCAAC
CAGAGAGGGATGACTACTTTTCTACCGCTCCTCCAGAACAACGAATTATTATAATGTACTATAATGAT
ACAATCGTGGAGCGCATAATTAATCCACCCGGGGTACTAGATGTATGGGCAACATTGAACCCAGGAAC
AGGAAGCGGGGTATATTATTTAGGTTGGGTACTCTTTCCAATATATGGCGGCGTGATTAAAGATACGA
GTTTATGGAATAATCAAGCAAATAAATACTTTATCCCCCAGATGGTTGCTGCTCTCTGCTCACAAAACC
AGGCAACTCAAGTCCAAAATGCTAAGTCATCATACTATAGCAGCTGGTTTGGCAATCGAATGATTCAG
TCTGGGATCCTGGCATGTCCTCTTCAACAGGATCTAACCAATGAGTGTTTAGTTCTGCCCTTTTCTAAT
GATCAGGTGCTTATGGGTGCTGAAGGGAGATTATACATGTATGGTGACTCGGTGTATTACTACCAAAG
AAGCAATAGTTGGTGGCCTATGACCATGCTGTATAAGGTAACCATAACATTCACTAATGGTCAGCCAT
CTGCTATATCAGCTCAGAATGTGCCCACACAGCAGGTCCCTAGACCTGGGACAGGAAGCTGCTCTGCA
ACAAATAGATGTCCCGGTTTTTGCTTGAAAGGAGTGTATGCTGATGCCTGGTTACTGACCAACCCTTCG
TCTACCAGTACATTTGGATCAGAAGCAACCTTCACTGGTTCTTATCTCAACGCAGCAACTCAGCGTATC
AATCCGACGATGTATATCGCGAACAACACACAGATCATAAGCTCACAGCAATTTGGATCAAGCGGTCA
AGAAGCAGCATATGGCCACACAACTTGTTTTAGGGACACAGGCTCTGTTATGGTATACTGTATCTATAT
TATTGAATTGTCCTCATCTCTCTTAGGACAATTTCAGATTGTCCCATTTATCCGTCAGGTGACACTATCC
TAAAGGCAGAAGCCTCCAGGTCTGACCCAGCCAATCAAAGCATTATACCAGACCATGGAATGCATACC
AAACATTATTGACACTAATGACACACAAAATTGGTTTTAAGAAAAACCAAGAGAACAATAGGCCAGA
ATGGCTGGGTCTCGGGAGATATTACTCCCTGAAGTCCATCTCAATTCACCAATTGTAAAGCATAAGCT
ATACTATTACATTCTACTTGGAAACCTCCCAAATGAGATCGACATTGACGATTTAGGTCCATTACATAA
TCAAAATTGGAATCAAATAGCACATGAAGAGTCTAACTTAGCCCAACGCTTGGTAAATGTAAGAAATT
TTCTAATTACCCACATCCCTGATCTTAGAAAGGGCCATTGGCAAGAGTATGTCAATGTAATACTGTGGC
CGCGAATTCTTCCCTTGATCCCGGATTTTAAAATCAATGACCAATTGCCTCTACTCAAAAATTGGGACA
AGTTAGTTAAAGAATCATGTTCAGTAATCAATGCGGGTACTTCCCAGTGCATTCAGAATCTCAGCTATG
GACTGACAGGTCGTGGGAACCTCTTTACACGATCACGTGAACTCTCTGGTGACCGCAGGGATATTGAT
CTTAAGACGGTTGTGGCAGCATGGCATGACTCAGACTGGAAAAGAATAAGTGATTTTTGGATTATGAT
CAAATTCCAGATGAGACAATTAATTGTTAGGCAAACAGATCATAATGATCCTGATTTAATCACGTATA
TCGAAAATAGAGAAGGCATAATCATCATAACCCCTGAACTGGTAGCATTATTTAACACTGAGAATCAT
ACACTAACATACATGACCTTTGAAATTGTACTGATGGTTTCAGATATGTACGAAGGTCGTCACAACATT
TTATCACTATGCACAGTTAGCACTTACCTGAATCCTCTGAAGAAAAGAATAACATATTTATTGAGCCTT
GTAGATAACTTAGCTTTTCAGATAGGTGATGCTGTATATAACATAATTGCTTTGCTAGAATCCTTTGTA
TATGCACAGTTGCAAATGTCAGATCCCATCCCAGAACTCAGAGGACAATTCCATGCATTCGTATGTTCT
GAGATTCTTGATGCACTAAGGGGAACTAATAGTTTCACCCAGGATGAATCAAGAACTGTGACAACCAA
TTTGATATCCCCATTCCAAGATCTGACCCCAGATCTTACGGCTGAATTGCTCTGTATAATGAGGCTTTG
GGGACACCCCATGCTCACCGCCAGTCAAGCTGCGGGAAAGGTACGCGAGTCCATGTGTGCTGGAAAA
GTATTAGACTTTCCCACCATTATGAAAACACTAGCCTTTTTCCATACTATTCTGATCAATGGATACAGG
AGGAAGCATCATGGAGTATGGCCACCCTTAAACTTACCGGGTAATGCTTCAAAGGGTCTCACGGAACT
TATGAATGACAATACTGAGATAAGCTATGAATTCACACTTAAGCATTGGAAGGAAATCTCTCTTATAA
AATTCAAGAAATGTTTTGATGCAGACGCAGGTGAGGAACTCAGTATATTTATGAAAGATAAAGCAATT
AGTGCCCCAAAACAAGATTGGATGAGTGTGTTTAGAAGAAGCCTAATCAAACAGCGCCATCAGCATC
ATCAGGTCCCCCTACCAAATCCATTCAATCGACGGCTATTGCTAAACTTTCTCGGAGATGACAAATTCG
ACCCGAATGTGGAGCTACAGTATGTAACATCAGGTGAGTATCTACATGATGACACGTTTTGTGCATCA
TATTCACTAAAAGAGAAGGAAATTAAACCTGATGGTCGAATTTTTGCAAAGTTGACTAAGAGAATGAG
ATCATGTCAAGTTATAGCAGAATCTCTTTTAGCGAACCATGCTGGGAAGTTAATGAAAGAGAATGGTG
TTGTGATGAATCAGCTATCATTAACAAAATCACTATTAACAATGAGTCAGATTGGAATAATATCCGAG
AAAGCTAGAAAATCGACTCGAGATAACATAAATCAACCTGGTTTCCAGAATATCCAGAGAAATAAAT
CACATCACTCCAAGCAAGTCAATCAGCGAGATCCAAGTGATGACTTTGAATTGGCAGCATCTTTTTTA
ACTACTGATCTCAAAAAATATTGTTTACAATGGAGGTACCAGACAATTATCCCATTTGCTCAATCATTA
AACAGAATGTATGGTTATCCTCATCTCTTTGAGTGGATTCACTTACGGCTAATGCGTAGTACACTTTAC
GTGGGGGATCCCTTCAACCCACCAGCAGATACCAGTCAATTTGATCTAGATAAAGTAATTAATGGAGA
TATCTTCATTGTATCACCCAGAGGTGGAATTGAAGGGCTATGTCAAAAGGCTTGGACAATGATATCTA
TCTCTGTGATAATTCTATCTGCCACAGAGTCTGGCACACGAGTAATGAGTATGGTGCAGGGAGATAAT
CAAGCAATTGCTGTCACCACACGAGTACCAAGGAGCCTGCCGACTCTTGAGAAAAAGACTATTGCTTT
TAGATCTTGTAATCTATTCTTTGAGAGGTTAAAATGTAATAATTTTGGATTAGGTCACCATTTGAAAGA
ACAAGAGACTATCATTAGTTCTCACTTCTTTGTTTATAGCAAGAGAATATTCTATCAGGGGAGGATTCT
AACGCAAGCCTTAAAAAATGCTAGTAAGCTCTGCTTGACAGCTGATGTCCTAGGAGAATGTACCCAAT
CATCATGTTCTAATCTTGCAACTACTGTCATGAGGTTAACTGAGAATGGTGTTGAAAAAGATATCTGTT
TCTACTTGAATATCTATATGACCATCAAACAGCTCTCCTATGATATCATCTTCCCTCAAGTGTCAATTCC
TGGAGATCAGATCACATTAGAATACATAAATAATCCACACCTGGTATCACGATTGGCTCTTCTGCCATC
CCAGCTAGGAGGTCTAAACTACCTGTCATGCAGTAGGCTGTTCAATCGAAACATAGGCGACCCGGTGG
TTTCCGCAGTTGCAGATCTTAAGAGATTAATTAAATCAGGATGTATGGATTACTGGATCCTTTATAACT
TATTAGGGAGAAAACCGGGAAACGGCTCATGGGCTACTTTAGCAGCTGACCCGTACTCAATCAATATA
GAGTATCAATACCCCCCAACTACAGCTCTTAAGAGGCACACCCAACAAGCTCTGATGGAACTCAGTAC
GAATCCAATGTTACGTGGCATATTCTCTGACAATGCACAGGCAGAAGAAAATAATCTTGCTAGATTTC
TCCTGGATAGGGAGGTGATCTTTCCGCGTGTAGCTCACATCATCATTGAGCAAACCAGTGTCGGGAGG
AGAAAACAGATTCAAGGATATTTGGATTCAACTAGATCGATAATGAGTAAATCACTAGAAATTAAGCC
CTTGTCCAATAGGAAGCTTAATGAAATACTGGATTACAACATCAATTACCTAGCTTACAATTTGGCATT
ACTCAAGAATGCTATTGAACCTCCGACTTATTTGAAAGCAATGACTCTTGAAACATGTAGCATCGACA
TTGCAAGGAGCCTCCGGAAGCTCTCCTGGGCCCCACTCTTGGGTGGGAGAAATCTTGAAGGATTAGAG
ACGCCAGATCCCATTGAAATTACTGCAGGAGCATTAATTGTTGGATCGGGCTACTGTGAACAGTGTGC
TGCAGGAGACAATCGATTCACATGGTTTTTCTTGCCATCTGGTATCGAGATAGGAGGGGATCCCCGTG
ATAATCCTCCTATCCGTGTACCGTACATTGGCTCCAGGACTGATGAGAGGAGGGTAGCCTCAATGGCA
TACATCAGGGGTGCCTCGAGTAGCCTAAAAGCAGTTCTTAGACTGGCGGGAGTGTACATCTGGGCATT
CGGAGATACTCTGGAGAATTGGATAGATGCACTGGATTTGTCTCACACTAGAGTTAACATCACACTTG
AACAGCTGCAATCCCTCACCCCACTTCCAACCTCTGCCAATCTAACCCATCGGTTGGATGATGGCACA
ACTACCCTAAAGTTTACTCCTGCGAGCTCTTACACCTTTTCAAGTTTCACTCATATATCAAATGATGAG
CAATACCTGACAATTAATGACAAAACTGCAGATTCAAATATAATCTACCAACAGTTAATGATCACTGG
ACTCGGAATTTTAGAAACATGGAATAATCCCCCAATCAATAGAACATTCGAAGAATCTACCCTACATT
TGCACACTGGTGCATCATGTTGTGTCCGACCTGTGGACTCCTGCATCATCTCAGAAGCATTAACAGTCA
AGCCACATATTACAGTACCGTACAGCAATAAATTTGTATTTGATGAAGACCCGCTATCTGAATATGAG
ACTGCAAAACTGGAATCGTTATCATTTCAAGCCCAATTAGGCAACATTGATGCTGTAGATATGACAGG
TAAATTAACATTATTGTCCCAATTCACTGCAAGGCAGATTATTAATGCAATCACTGGACTCGATGAGTC
TGTCTCTCTTACTAATGATGCCATTGTTGCATCAGACTATGTCTCCAATTGGATTAGTGAATGCATGTA
TACCAAATTAGATGAATTATTTATGTATTGTGGGTGGGAACTACTATTGGAACTATCCTATCAAATGTA
TTATCTGAGGGTAGTTGGGTGGAGTAATATAGTGGATTATTCTTACATGATCTTGAGAAGAATCCCGG
GTGCAGCATTAAACAATCTGGCATCTACATTAAGTCATCCAAAACTTTTCCGACGAGCTATCAACCTA
GATATAGTTGCCCCCTTAAATGCTCCTCATTTTGCATCTCTGGACTACATCAAGATGAGTGTGGATGCA
ATACTCTGGGGCTGTAAAAGAGTCATCAATGTGCTCTCCAATGGAGGGGACTTAGAATTAGTTGTGAC
ATCTGAAGATAGCCTTATTCTCAGTGACCGATCCATGAATCTCATTGCAAGGAAATTAACTTTATTATC
ACTGATTCACCATAATGGTTTGGAACTACCAAAGATTAAGGGGTTCTCTCCTGATGAGAAGTGTTTCGC
TTTGACAGAATTTTTGAGGAAAGTGGTGAACTCAGGGTTGAGTTCAATAGAGAACCTATCAAATTTTA
TGTACAATGTGGAGAACCCACGGCTTGCAGCATTCGCCAGCAACAATTACTACCTGACCAGAAAATTA
TTGAATTCAATACGAGATACTGAGTCGGGTCAAGTAGCAGTCACCTCATATTATGAATCATTAGAATA
TATTGATAGTCTTAAGCTAACCCCACATGTGCCTGGTACCTCATGCATTGAGGATGATAGTCTATGTAC
AAATGATTACATAATCTGGATCATAGAGTCTAATGCAAACTTGGAGAAGTATCCAATTCCAAATAGCC
CTGAGGATGATTCCAATTTCCATAACTTTAAGTTGAATGCTCCATCGCACCATACCTTACGCCCATTAG
GGTTGTCATCAACTGCTTGGTATAAGGGTATAAGCTGTTGCAGGTACCTTGAGCGATTAAAGCTACCA
CAAGGTGATCATTTATATATTGCAGAAGGTAGTGGTGCCAGTATGACAATCATAGAATACCTATTCCC
AGGAAGAAAGATATATTACAATTCTTTATTTAGTAGTGGTGACAATCCCCCACAAAGAAATTATGCAC
CAATGCCTACTCAGTTCATTGAGAGTGTCCCATACAAGCTCTGGCAAGCACACACAGATCAATATCCC
GAGATTTTTGAGGACTTCATCCCTCTATGGAACGGAAATGCCGCCATGACTGACATAGGAATGACAGC
TTGTGTAGAATTCATCATCAATCGAGTCGGCCCAAGGACTTGCAGTTTAGTACATGTAGATTTGGAATC
AAGTGCAAGCTTAAATCAACAATGCCTGTCAAAGCCGATAATTAATGCTATCATCACTGCTACAACTG
TTTTGTGCCCTCATGGGGTGCTTATTCTGAAATATAGTTGGTTGCCATTTACTAGATTTAGTACTTTGAT
CACTTTCTTATGGTGCTACTTTGAGAGAATCACTGTTCTTAGGAGCACATATTCTGGTCCAGCTAATCA
TGAGGTTTATTTAATTTGTATCCTTGCCAACAACTTTGCATTCCAGACTGTCTCGCAGGCAACAGGAAT
GGCGATGACTTTAACCGATCAAGGGTTTACTTTGATATCACCTGAAAGAATAAATCAGTATTGGGATG
GTCACTTGAAGCAAGAACGTATCGTAGCAGAAGCAATTGATAAGGTGGTTCTAGGAGAAGATGCTCTA
TTCAATTCGAGTGATAATGAATTAATTCTCAAATGTGGAGGGACACCAAATGCACGGAATCTTATCGA
TATCGAGCCAGTCGCAACTTTCATAGAATTTGAACAACTGATCTGCACAATGTTGACAACCCACTTGA
AGGAAATAATTGATATAACAAGGTCTGGAACCCAGGATTATGAAAGTTTATTACTCACTCCTTACAAT
TTAGGTCTTCTTGGTAAAATCAGTACGATAGTGAGATTATTAACAGAAAGGATTCTAAATCATACTATC
AGGAATTGGTTGATCCTCCCACCTTCGCTCCGGATGATCGTGAAGCAGGACTTGGAATTCGGCATATTC
AGGATTACTTCCATCCTCAATTCTGATCGGTTCCTGAAGCTTTCTCCAAATAGGAAATACTTGATTACA
CAATTAACTGCAGGCTACATTAGGAAATTGATTGAGGGGGATTGTAATATCGATCTAACCAGACCTAT
CCAAAAACAAATCTGGAAAGCATTAGGTTGTGTAGTCTATTGTCACGATCCAGTAGATCAAAGGGAAT
CAACAGAGTTTATTGATATAAATATTAATGAAGAAATAGACCGCGGGATCGATGGCGAGGAAATCTA
AATATATCAAGAATCAGAATTAGTTTAAGAAAAAAGAAGAGGATTAATCTTGGTTTTCCCCTTGGT.

15. CVL65

The nucleic acid sequence for CVL65 is:

(SEQ ID NO: 74)
ACCAGGGGGAAAACGAAGTGGTGACTCAAATCATCGAAGACCCTCGAGATTACATAGGTCCGGAACC
TATGGCCTTCGTGACCGACCTCGAGTCAGAGTAGTTCAATAAGGACCTATCAAGTTTGGGCAATTTTTC
GTCCCCGACACAAAAATGTCATCCGTGCTTAAAGCATATGAGCGATTCACACTCACTCAAGAACTGCA
AGATCAGAGTGAGGAAGGTACAATCCCACCTACAACACTAAAACCGGTAATCAGGGTATTTATACTAA
CCTCTAATAACCCAGAGCTAAGATCCCGGCTTCTTCTATTCTGCCTACGGATTGTTCTCAGTAATGGTG
CAAGGGATTCCCATCGCTTTGGAGCATTACTTACAATGTTTTCGCTACCATCAGCCACAATGCTCAATC
ATGTCAAATTAGCTGACCAGTCACCAGAAGCTGATATCGAAAGGGTAGAGATCGATGGCTTTGAGGA
GGGATCATTCCGCTTAATCCCCAATGCTCGTTCAGGTATGAGCCGTGGAGAGATCAATGCCTATGCTG
CACTTGCAGAAGATCTACCTGACACACTAAACCATGCAACACCTTTTGTTGATTCCGAAGTCGAGGGA
ACTGCATGGGATGAGATTGAGACTTTCTTAGATATGTGTTACAGTGTCCTAATGCAGGCATGGATAGT
GACTTGCAAGTGCATGACTGCGCCAGACCAACCTGCTGCTTCTATTGAGAAACGCCTGCAAAAATATC
GTCAGCAAGGCAGGATCAACCCGAGATATCTCCTGCAACCGGAGGCTCGAAGAATAATCCAGAATGT
AATCCGAAAGGGAATGGTGGTCAGACATTTCCTCACCTTTGAACTGCAGCTTGCCCGAGCACAAAGCC
TTGTATCAAATAGGTATTATGCTATGGTAGGGGATGTTGGAAAGTATATAGAGAATTGTGGAATGGGA
GGCTTCTTTTTGACACTAAAATATGCATTAGGAACCAGATGGCCCACACTTGCTTTAGCTGCATTTTCA
GGAGAGCTAACAAAGCTAAAGTCCCTCATGGCATTATACCAGACCCTTGGTGAGCAGGCCCGATATTT
GGCCCTATTGGAGTCACCACATTTGATGGATTTTGCTGCAGCAAACTACCCACTGCTATATAGCTATGC
TATGGGAATAGGCTATGTGTTAGATGTCAACATGAGGAACTACGCTTTCTCCAGATCATACATGAATA
AGACATATTTCCAATTGGGAATGGAAACTGCAAGAAAACAACAGGGTGCAGTTGACATGAGGATGGC
AGAAGATCTCGGTCTAACTCAAGCCGAACGCACCGAGATGGCAAATACACTTGCCAAATTGACCACA
GCAAATCGAGGGGCAGACACCAGGGGAGGAGTCAACCCGTTCTCATCTATCACTGGGACAACTCAGG
TGCCCGCTGCAGCAACAGGTGACACATTCGAGAGTTACATGGCAGCGGATCGACTGAGGCAGAGATA
TGCTGATGCAGGCACCCACGATGATGAGATGCCACCATTGGAAGAGGAGGAAGAGGACGACACATCT
GCAGGTCCACGCACTGGACTAACTCTTGAACAAGTGGCCTTGGACATCCAGAACGCAGCAGTTGGAGC
TCCCATCCATACAGATGACCTGAATGCCGCACTGGGTGATCTTGACATCTAGACAATTCAGATCCCAA
TCTTAAATCGACACACCTAATTGACCAGTTAGATGGAACTACAGTGGATTCCATAAGGTTCCTGCCTAC
CATCGGCTTTTAAGAAAAAAATAGGCCCGGACGGGTTAGCAACAAGCGACTGCCGATGCCAATAACA
CAATCCACAATCTACAATGGATCCCACTGATCTGAGCTTCTCCCCAGATGAGATCAATAAGCTCATAG
AGACAGGCCTGAATACTGTGGAGTATTTTACTTCCCAACAAGTCACAGGAACATCCTCTCTTGGAAAG
AATACAATACCACCAGGGGTCACAGGACTACTAACCAATGCTGCAGAGGCAAAGATCCAAGAGTCAA
CCAACCATCAGAAGGGTTCAGTTGGTGGGGGCGCAAAACCAAAGAAACCGCGACCAAAAATTGCCAT
TGTGCCAGCAGATGACAAAACGGTGCCCGGAAAGCCGATCCCAAACCCTCTATTAGGTCTGGACTCCA
CCCCGAGCACCCAAACTGTGCTTGATCTAAGTGGGAAAACATTACCATCAGGATCCTATAAGGGGGTT
AAACTTGCGAAATTTGGAAAAGAAAATCTGATGACACGGTTCATCGAGGAACCCAGAGAGAATCCTA
TCGCAACCAATTCCCCCATCGATTTTAAGAGGGGCAGGGATACCGGCGGGTTCCATAGAAGGGAGTAC
TCAATCGGATGGGTGGGAGATGAAGTCAAGGTCACTGAGTGGTGCAATCCATCCTGTTCTCCAATCAC
CGCTGCAGCAAGGCGATTTGAATGCACTTGTCACCAGTGTCCAGTCACTTGCTCTGAATGTGAACGAG
ATACTTAATACAGTGAGAAATTTGGACTCTCGGATGAATCAACTGGAGACAAAAGTAGATCGCATTCT
CTCATCTCAGTCTCTAATCCAGACCATCAAGAATGACATAGTTGGACTTAAAGCAGGGATGGCTACTT
TAGAAGGAATGATTACAACTGTGAAAATCATGGACCCGGGAGTTCCCAGTAATGTTACTGTGGAAGAT
GTACGCAAGAAACTAAGTAACCATGCTGTTGTTGTGCCAGAATCATTCAATGATAGTTTCTTGACTCAA
TCTGAAGATGTAATTTCACTTGATGAGTTGGCTCGACCAACTGCAACAAGTGTTAAGAAGATTGTCAG
GAAGGTTCCTCCTCAGAAGGATCTGACTGGATTGAAGATCACACTAGAGCAATTGGCAAAGGATTGCA
TCAGCAAACCGAAGATGAGGGAAGAGTATCTCCTCAAAATCAACCAGGCTTCCAGTGAGGCTCAGCT
AATTGACCTCAAGAAAGCAATCATCCGCAGTGCAATTTGATCAAGAAACACCCAATTACACTACACTG
GTATGACACTGTACTAACCCTGAGGGTTTTAGAAAAAACGATTAACGATAAATAAGCCCGAACACTAC
ACACCACCCGAGGCAGCCATGCCATCCATCAGCATCCCCGCAGACCCCACCAATCCACGTCAATCAAT
AAAAGCGTTCCCAATTGTGATCAACCGTGATGGGGGTGAGAAAGGTCGCTTGGTTAAACAACTACGCA
CAACCTACTTGAATGACCTAGATACTCATGAGCCACTGGTGACATTCGTAAATACTTATGGATTCATCT
ACGAACAGGATCGGGGAAATACCATTGTCGGAGAGGATCAACATGGGAAGAAAAGAGAGGCTGTGA
CTGCTGCAATGGTTACCCTTGGATGTGGGCCTAATCTACCATCATTAGGGAATGTCCTGGGACAACTG
AGTGAATTCCAGGTCATTGTTAGGAAGACATCCAGCAAAGCGGAAGAGATGGTCTTTGAAATTGTTAA
GTATCCGAGAATATTTCGGGGTCATACATTAATCCAGAAAGGACTAGTCTGTGTCTCCGCAGAAAAAT
TTGTTAAGTCACCAGGGAAAGTACAATCTGGAATGGACTATCTCTTCATTCCGACATTTCTGTCAGTGA
CTTACTGTCCAGCTGCAATCAAATTTCAGGTACCTGGCCCCATGTTGAAAATGAGATCAAGATACACT
CAGAGCTTACAACTTGAACTAATGATAAGAATCCTGTGTAAGCCCGATTCGCCACTTATGAAGGTCCA
TATCCCTGACAAGGAAGGAAGAGGATGTCTTGTATCAGTATGGTTGCATGTATGCAATATCTTCAAAT
CAGGAAACAAGAATGGCAGTGAGTGGCAGGAATACTGGATGAGAAAGTGTGCTAACATGCAACTTGA
AGTGTCGATTGCAGATATGTGGGGACCAACTATCATAATTCATGCCAGAGGTCACATTCCCAAAAGTG
CTAAGTTGTTTTTTGGAAAGGGTGGATGGAGCTGCCATCCACTTCACGAAGTTGTTCCAAGTGTCACTA
AAACACTATGGTCCGTGGGCTGTGAGATTACAAAGGCGAAGGCAATAATACAAGAGAGTAGCATCTC
TCTTCTCGTGGAGACTACTGACATCATAAGTCCAAAAGTCAAAATTTCATCTAAGCATCGCCGCTTTGG
GAAATCAAATTGGGGTCTGTTCAAGAAAACCAAATCACTGCCCAACCTGACGGAGCTGGAATGACTG
ACCCCCAATCGAGACTACACCACCTCAAACTATAGGTGGGTGGTACCTCAGTGATTAATCTCGTAAGC
ACTGATCGTAGGCTACAACACACTAATATTATCCAGATTAGAGAGCTTAATTAGCTCTGTATTAATAAT
AACACTACTATTCCAATAACTGGAATCACCAGCTTGATTTATCTCCAAAATGATTCAAAGAAAACAAA
TCATATTAAGACTATCCTAAGCACGAACCCATATCGTCCTTCAAATCATGGGTACTATAATTCAATTTC
TGGTGGTCTCCTGTCTATTGGCAGGAGCAGGCAGTCTTGATCCAGCAGCCCTCATGCAAATCGGTGTC
ATTCCAACAAATGTCCGGCAACTTATGTATTATACTGAGGCCTCATCAGCATTCATTGTTGTGAAGTTA
ATGCCTACAATTGACTCGCCGATTAGTGGATGTAATATAACATCAATTTCAAGCTATAATGCAACAGT
GACAAAACTCCTACAGCCGATCGGTGAGAATTTGGAGACGATTAGGAACCAGTTGATTCCAACTCGGA
GGAGACGCCGGTTTGCAGGGGTGGTGATTGGATTAGCTGCATTAGGAGTAGCTACTGCCGCACAGGTC
ACTGCCGCAGTAGCACTAGTAAAGGCAAATGAAAATGCTGCGGCTATACTCAATCTCAAAAATGCAAT
CCAAAAAACAAATGCAGCAGTTGCAGATGTGGTCCAGGCCACACAATCACTAGGAACaGCAGTTCAAG
CAGTTCAAGATCACATAAACAGTGTGGTAAGTCCAGCAATTACAGCAGCCAATTGTAAAGCCCAAGAT
GCTATCATTGGCTCAATCCTCAATCTCTATTTGACCGAGTTGACAACTATCTTCCACAATCAAATTACA
AACCCTGCATTGAGTCCTATTACAATTCAAGCTTTAAGGATCCTACTAGGGAGTACCTTGCCGACTGTG
GTCGAAAAATCTTTCAATACCCAGATAAGTGCGGCTGAGCTTCTCTCATCAGGGTTATTGACAGGCCA
GATTGTGGGATTAGATTTGACCTATATGCAGATGGTCATAAAAATTGAGCTGCCAACTTTAACTGTAC
AACCTGCAACCCAGATCATAGATCTGGCCACCATTTCTGCATTCATTAACAATCAAGAAGTCATGGCC
CAATTACCAACACGTGTTATTGTGACTGGCAGCTTGATCCAAGCCTATCCCGCATCGCAATGCACTATT
ACACCCAACACTGTGTACTGTAGGTATAATGATGCCCAAGTACTCTCAGATGATACGATGGCTTGCCT
CCAAGGTAACTTGACAAGATGCACCTTCTCTCCAGTGGTTGGGAGCTTTCTCACTCGATTCGTGCTGTT
CGATGGAATAGTTTATGCAAATTGCAGGTCGATGTTGTGCAAGTGCATGCAGCCTGCTGCTGTGATCCT
ACAGCCGAGTTCATCCCCTGTAACTGTCATTGACATGTACAAATGTGTGAGTCTGCAGCTTGACAATCT
CAGATTCACCATCACTCAATTGGCCAATGTAACCTACAATAGCACCATCAAGCTTGAAACATCCCAGA
TCTTGCCTATTGATCCGTTGGATATATCCCAGAATCTAGCTGCGGTGAATAAGAGTCTAAGTGATGCAC
TACAACACTTAGCACAAAGTGACACATACCTTTCTGCAATCACATCAGCTACGACTACAAGTGTATTA
TCCATAATAGCAATCTGTCTTGGATCGTTAGGTTTAATATTAATAATCTTGCTCAGTGTAGTTGTGTGG
AAGTTATTGACCATTGTCGCTGCTAATCGAAATAGAATGGAGAATTTTGTTTATCATAATTCAGCATTC
CACCACTCACGATCTGATCTCAGTGAGAAAAATCAACCTGCAACTCTTGGAACAAGATAAGACAGTCA
TCCATTAGTAATTTTTAAGAAAAAAACGATAGGACCGAAACTAGTATTGAAAGAACCGTCTCGGTCAA
TCTAGGTAATCGAGCTGcTACCGTCTCGGAAAGCTCAAATCGCGCGCCACCatgtcagataacggacca
cagaaccagaggaacgcacccaggattactttcggaggaccaagcgatagcaccgggagcaaccagaatg
gagagcggagcggagcaagatccaagcagagacggccccagggcctgccaaacaataccgcatcctgg
ttcaccgccctgacacagcacggcaaggaggacctgaagtttccaaggggacagggagtgcctatcaa
caccaatagctcccctgacgatcagatcggctactataggagggcaacaaggagaatcaggggaggcgac
ggcaagatgaaggatctgagcccacgctggtacttctactatctgggaaccggacctgaggcaggcct
gccatatggcgccaacaaggacggaatcatctgggtggcaaccgagggcgccctgaacacaccaaagga
tcacatcggcacaagaaatcccgccaacaatgcagcaatcgtgctgcagctgccacagggaaccacact
gcccaagggcttttacgcagagggctctcggggaggcagccaggcatctagcagatcctctagccggagc
agaaactcctctaggaattccaccccaggaagctccaggggcacatcccctgcccgcatggcaggaa
acggaggcgacgccgccctggccctgctgctgctggatcgcctgaatcagctggagtccaagatgtctgg
caagggacagcagcagcagggacagaccgtgacaaagaagtccgccgccgaggcctctaagaagccaagg
cagaagcgcaccgccacaaaggcctacaacgtgacccaggccttcggcaggcgcggaccagagca
gacacagggcaattttggcgaccaggagctgatcaggcagggaaccgattataagcactggcctca
gatcgcccagttcgccccatctgccagcgccttctttggcatgtctagaatcggcatggaggtgac
ccccagcggcacatggctgacctacacaggcgccatcaagctggacgataaggaccctaacttcaagg
atcaggtcatcctgctgaacaagcacatcgacgcctataagacctttccccctacagagcccaagaa
ggacaagaagaagaaggccgatgagacacaggccctgcctcagaggcagaagaagcagcagaccgtga
cactgctgccagccgccgatctggacgatttctccaaacagctgcagcagagcatgtccagtgc
cgactccacccaggcttgaGCTAGCCTCCTGCCATACTTCCTACTCACATCATA
TCTATTTTAAAGAAAAAATAGGCCCGAACACTAATCGTGCCGGCAGTGCCACTGCACACACAA
CACTACACATACAATACACTACAATGGTTGCAGAAGATGCCCCTGTTAGGGGCACTTGCCGAGTATTA
TTTCGAACAACAACTTTAATTTTTCTATGCACACTATTAGCATTAAGCATCTCTATCCTTTATGAGAGTT
TAATAACCCAAAAGCAAATCATGAGCCAAGCAGGCTCAACTGGATCTAATTCTAGATTAGGAAGTATC
ACTGATCTTCTTAATAATATTCTCTCTGTCGCAAATCAGATTATATATAACTCTGCAGTCGCTCTACCTC
TACAATTGGACACTCTTGAATCAACACTCCTTACAGCCATTAAGTCTCTTCAAACAAGTGACAAGCTA
GAACAGAACTGCTCGTGGGGTGCTGCACTGATTAATGATAATAGATACATTAATGGCATCAATCAGTT
CTATTTCTCAATTGCTGAGGGTCGCAAGCTGACACTTGGCCCACTTCTTAATATACCTAGTTTCATTCC
AACTGCCACGACACCAGAGGGCTGCACCAGGATCCCATCATTCTCGCTCACCAAGACACACTGGTGTT
ATACACACAATGTTATCCTGAATGGATGCCAGGATCATGTATCCTCAAATCAATTTGTTTCCATGGGAA
TCATTGAACCCACTTCTGCCGGGTTTCCATCCTTTCGAACCCTAAAGACTCTATATCTCAGCGATGGGG
TCAATCGTAAGAGCTGCTCTATCAGTACAGTTCCGGGGGGTTGTATGATGTACTGTTTCGTCTCTACTC
AACCAGAGAGGGATGACTACTTTTCTACCGCTCCTCCAGAACAACGAATTATTATAATGTACTATAAT
GATACAATCGTGGAGCGCATAATTAATCCACCCGGGGTACTAGATGTATGGGCAACATTGAACCCAGG
AACAGGAAGCGGGGTATATTATTTAGGTTGGGTACTCTTTCCAATATATGGCGGCGTGATTAAAGATA
CGAGTTTATGGAATAATCAAGCAAATAAATACTTTATCCCCCAGATGGTTGCTGCTCTCTGCTCACAAA
ACCAGGCAACTCAAGTCCAAAATGCTAAGTCATCATACTATAGCAGCTGGTTTGGCAATCGAATGATT
CAGTCTGGGATCCTGGCATGTCCTCTTCAACAGGATCTAACCAATGAGTGTTTAGTTCTGCCCTTTTCT
AATGATCAGGTGCTTATGGGTGCTGAAGGGAGATTATACATGTATGGTGACTCGGTGTATTACTACCA
AAGAAGCAATAGTTGGTGGCCTATGACCATGCTGTATAAGGTAACCATAACATTCACTAATGGTCAGC
CATCTGCTATATCAGCTCAGAATGTGCCCACACAGCAGGTCCCTAGACCTGGGACAGGAAGCTGCTCT
GCAACAAATAGATGTCCCGGTTTTTGCTTGAAAGGAGTGTATGCTGATGCCTGGTTACTGACCAACCCT
TCGTCTACCAGTACATTTGGATCAGAAGCAACCTTCACTGGTTCTTATCTCAACGCAGCAACTCAGCGT
ATCAATCCGACGATGTATATCGCGAACAACACACAGATCATAAGCTCACAGCAATTTGGATCAAGCGG
TCAAGAAGCAGCATATGGCCACACAACTTGTTTTAGGGACACAGGCTCTGTTATGGTATACTGTATCT
ATATTATTGAATTGTCCTCATCTCTCTTAGGACAATTTCAGATTGTCCCATTTATCCGTCAGGTGACACT
ATCCTAAACCTGCTATAGGCTATCCACTGCATCATCTCTTTTTAAGAAAAAAATAGGCCCGGACGGGTT
AGCAACAAGCGACTGCCGGTGCCAACAACACAATCCACAATCTACAGTCGACGCGGCCGCCACCatgttc
gtcttcctggtcctgctgcctctggtctcctcacagtgcgtcaatctgagaactcggactcagctgccac
ctgcttatactaatagcttcaccagaggcgtgtactatcctgacaaggtgtttagaagctccgtgctgc
actctacacaggatctgtttctgccattctttagcaacgtgacctggttccacgccatccacgtgagc
ggcaccaatggcacaaagcggttcgacaatcccgtgctgccttttaacgatggcgtgtacttcgcctct
accgagaagagcaacatcatcagaggctggatctttggcaccacactggactccaagacacagtctc
tgctgatcgtgaacaatgccaccaacgtggtcatcaaggtgtgcgagttccagttttgtaatgatccct
tcctgggcgtgtactatcacaagaacaataagagctggatggagtccggcgtgtattctagcgccaa
caactgcacatttgagtacgtgagccagcctttcctgatggacctggagggcaagcagggcaatttcaa
gaacctgagggagttcgtgtttaagaatatcgacggctacttcaaaatctactctaagcacacccccatc
aacctggtgcgcgacctgcctcagggcttcagcgccctggagcccctggtggatctgcctatcggcatca
acatcacccggtttcagacactgctggccctgcacagaagctacctgacacccggcgactcctctagc
ggatggaccgccggcgctgccgcctactatgtgggctacctccagccccggaccttcctgctgaagtac
aacgagaatggcaccatcacagacgcagtggattgcgccctggaccccctgagcgagacaaagtgtaca
ctgaagtcctttaccgtggagaagggcatctatcagacatccaatttcagggtgcagccaaccgagt
ctatcgtgcgctttcctaatatcacaaacctgtgcccatttggcgaggtgttcaacgcaacccgcttc
gccagcgtgtacgcctggaataggaagcggatcagcaactgcgtggccgactatagcgtgctgtacaa
ctccgcctctttcagcacctttaagtgctatggcgtgtcccccacaaagctgaatgacctgtgctttac
caacgtctacgccgattctttcgtgatcaggggcgacgaggtgcgccagatcgcccccggccagacaggc
aagatcgcagactacaattataagctgccagacgatttcaccggctgcgtgatcgcctggaacagcaaca
atctggattccaaagtgggcggcaactacaattatagataccggctgtttagaaagagcaatctgaagcc
cttcgagagggacatctctacagaaatctaccaggccggcagcaagccttgcaatggcgtggagggc
tttaactgttatttcccactccagtcctacggcttccagcccacaaacggcgtgggctatcagcctta
ccgcgtggtggtgctgagctttgagctgctgcacgccccagcaacagtgtgcggccccaagaagtcca
ccaatctggtgaagaacaagtgcgtgaacttcaacttcaacggcctgaccggcacaggcgtgctgac
cgagtccaacaagaagttcctgccatttcagcagttcggcagggacatcgcagataccacagacgccgt
gcgcgacccacagaccctggagatcctggacatcacaccctgctctttcggcggcgtgagcgtgatcac
acccggcaccaatacaagcaaccaggtggccgtgctgtatcagggcgtgaattgtaccgaggtgc
ccgtggctatccacgccgatcagctgaccccaacatggcgggtgtacagcaccggctccaacgtct
tccagacaagagccggatgcctgatcggagcagagcacgtgaacaattcctatgagtgcgacatcc
caatcggcgccggcatctgtgcctcttaccagacccagacaaactctagaagaagagcccggagcgtg
gcctcccagtctatcatcgcctataccatgtccctgggcgccgagaacagcgtggcctactctaaca
atagcatcgccatcccaaccaacttcacaatctctgtgaccacagagatcctgcccgtgtccatgacca
agacatctgtggactgcacaatgtatatctgtggcgattctaccgagtgcagcaacctgctgctccag
tacggcagcttttgtacccagctgaatagagccctgacaggcatcgccgtggagcaggataagaacaca
caggaggtgttcgcccaggtgaagcaaatctacaagaccccccctatcaaggactttggcggcttcaa
tttttcccagatcctgcctgatccatccaagccttctaagcggagctttatcgaggacctgctgttc
aacaaggtgaccctggccgatgccggcttcatcaagcagtatggcgattgcctgggcgacatcgc
agccagggacctgatctgcgcccagaagtttaatggcctgaccgtgctgccacccctgctgacagatgag
atgatcgcacagtacacaagcgccctgctggccggcaccatcacatccggatggaccttcggcgcagg
agccgccctccagatcccctttgccatgcagatggcctataggttcaacggcatcggcgtgaccca
gaatgtgctgtacgagaaccagaagctgatcgccaatcagtttaactccgccatcggcaagatccag
gacagcctgtcctctacagccagcgccctgggcaagctccagaacgtggtgaatcagaacgcccaggcc
ctgaataccctggtgaagcagctgagcagcaacttcggcgccatctctagcgtgctgaatgacatcct
gagccggctggacaaggtggaggcagaggtgcagatcgaccggctgatcaccggccggctccagagc
ctccagacctatgtgacacagcagctgatcagggccgccgagatcagggccagcgccaatctggcag
caaccaagatgtccgagtgcgtgctgggccagtctaagagagtggacttttgtggcaagggctatc
acctgatgtccttccctcagtctgccccacacggcgtggtgtttctgcacgtgacctacgtgcccgcc
caggagaagaacttcaccacagcccctgccatctgccacgatggcaaggcccactttccaagggaggg
cgtgttcgtgtccaacggcacccactggtttgtgacacagcgcaatttctacgagccccagatcatcac
cacagacaacaccttcgtgagcggcaactgtgacgtggtcatcggcatcgtgaacaataccgtgtat
gatccactccagcccgagctggacagctttaaggaggagctggataagtatttcaagaatcacacctc
ccctgacgtggatctgggcgacatcagcggcatcaatgcctccgtggtgaacatccagaaggagatcg
accgcctgaacgaggtggctaagaatctgaacgagagcctgatcgacctccaggagctgggcaagta
tgagcagtacatcaagtggccctggtacatctggctgggcttcatcgccggcctgatcgccatcgtga
tggtgaccatcatgctgtgctgtatgacatcctgctgttcttgcctgaagggctgctgtagctgtggc
tcctgctgtAAGTTATTGACCATTGTCGTTGCTAATCGAAATAGAATGGAGAATTTTGTTTATCATAA
ATGAATTCGTACGAGGCAGAAGCCTCCAGGTCTGACCCAGCCAATCAAAGCATTATACCAGACCATGG
AATGCATACCAAACATTATTGACACTAATGACACACAAAATTGGTTTTAAGAAAAACCAAGAGAACA
ATAGGCCAGAATGGCTGGGTCTCGGGAGATATTACTCCCTGAAGTCCATCTCAATTCACCAATTGTAA
AGCATAAGCTATACTATTACATTCTACTTGGAAACCTCCCAAATGAGATCGACATTGACGATTTAGGTC
CATTACATAATCAAAATTGGAATCAAATAGCACATGAAGAGTCTAACTTAGCCCAACGCTTGGTAAAT
GTAAGAAATTTTCTAATTACCCACATCCCTGATCTTAGAAAGGGCCATTGGCAAGAGTATGTCAATGT
AATACTGTGGCCGCGAATTCTTCCCTTGATCCCGGATTTTAAAATCAATGACCAATTGCCTCTACTCAA
AAATTGGGACAAGTTAGTTAAAGAATCATGTTCAGTAATCAATGCGGGTACTTCCCAGTGCATTCAGA
ATCTCAGCTATGGACTGACAGGTCGTGGGAACCTCTTTACACGATCACGTGAACTCTCTGGTGACCGC
AGGGATATTGATCTTAAGACGGTTGTGGCAGCATGGCATGACTCAGACTGGAAAAGAATAAGTGATTT
TTGGATTATGATCAAATTCCAGATGAGACAATTAATTGTTAGGCAAACAGATCATAATGATCCTGATTT
AATCACGTATATCGAAAATAGAGAAGGCATAATCATCATAACCCCTGAACTGGTAGCATTATTTAACA
CTGAGAATCATACACTAACATACATGACCTTTGAAATTGTACTGATGGTTTCAGATATGTACGAAGGT
CGTCACAACATTTTATCACTATGCACAGTTAGCACTTACCTGAATCCTCTGAAGAAAAGAATAACATA
TTTATTGAGCCTTGTAGATAACTTAGCTTTTCAGATAGGTGATGCTGTATATAACATAATTGCTTTGCT
AGAATCCTTTGTATATGCACAGTTGCAAATGTCAGATCCCATCCCAGAACTCAGAGGACAATTCCATG
CATTCGTATGTTCTGAGATTCTTGATGCACTAAGGGGAACTAATAGTTTCACCCAGGATGAATCAAGA
ACTGTGACAACCAATTTGATATCCCCATTCCAAGATCTGACCCCAGATCTTACGGCTGAATTGCTCTGT
ATAATGAGGCTTTGGGGACACCCCATGCTCACCGCCAGTCAAGCTGCGGGAAAGGTACGCGAGTCCAT
GTGTGCTGGAAAAGTATTAGACTTTCCCACCATTATGAAAACACTAGCCTTTTTCCATACTATTCTGAT
CAATGGATACAGGAGGAAGCATCATGGAGTATGGCCACCCTTAAACTTACCGGGTAATGCTTCAAAGG
GTCTCACGGAACTTATGAATGACAATACTGAGATAAGCTATGAATTCACACTTAAGCATTGGAAGGAA
ATCTCTCTTATAAAATTCAAGAAATGTTTTGATGCAGACGCAGGTGAGGAACTCAGTATATTTATGAA
AGATAAAGCAATTAGTGCCCCAAAACAAGATTGGATGAGTGTGTTTAGAAGAAGCCTAATCAAACAG
CGCCATCAGCATCATCAGGTCCCCCTACCAAATCCATTCAATCGACGGCTATTGCTAAACTTTCTCGGA
GATGACAAATTCGACCCGAATGTGGAGCTACAGTATGTAACATCAGGTGAGTATCTACATGATGACAC
GTTTTGTGCATCATATTCACTAAAAGAGAAGGAAATTAAACCTGATGGTCGAATTTTTGCAAAGTTGA
CTAAGAGAATGAGATCATGTCAAGTTATAGCAGAATCTCTTTTAGCGAACCATGCTGGGAAGTTAATG
AAAGAGAATGGTGTTGTGATGAATCAGCTATCATTAACAAAATCACTATTAACAATGAGTCAGATTGG
AATAATATCCGAGAAAGCTAGAAAATCGACTCGAGATAACATAAATCAACCTGGTTTCCAGAATATCC
AGAGAAATAAATCACATCACTCCAAGCAAGTCAATCAGCGAGATCCAAGTGATGACTTTGAATTGGCA
GCATCTTTTTTAACTACTGATCTCAAAAAATATTGTTTACAATGGAGGTACCAGACAATTATCCCATTT
GCTCAATCATTAAACAGAATGTATGGTTATCCTCATCTCTTTGAGTGGATTCACTTACGGCTAATGCGT
AGTACACTTTACGTGGGGGATCCCTTCAACCCACCAGCAGATACCAGTCAATTTGATCTAGATAAAGT
AATTAATGGAGATATCTTCATTGTATCACCCAGAGGTGGAATTGAAGGGCTATGTCAAAAGGCTTGGA
CAATGATATCTATCTCTGTGATAATTCTATCTGCCACAGAGTCTGGCACACGAGTAATGAGTATGGTGC
AGGGAGATAATCAAGCAATTGCTGTCACCACACGAGTACCAAGGAGCCTGCCGACTCTTGAGAAAAA
GACTATTGCTTTTAGATCTTGTAATCTATTCTTTGAGAGGTTAAAATGTAATAATTTTGGATTAGGTCA
CCATTTGAAAGAACAAGAGACTATCATTAGTTCTCACTTCTTTGTTTATAGCAAGAGAATATTCTATCA
GGGGAGGATTCTAACGCAAGCCTTAAAAAATGCTAGTAAGCTCTGCTTGACAGCTGATGTCCTAGGAG
AATGTACCCAATCATCATGTTCTAATCTTGCAACTACTGTCATGAGGTTAACTGAGAATGGTGTTGAAA
AAGATATCTGTTTCTACTTGAATATCTATATGACCATCAAACAGCTCTCCTATGATATCATCTTCCCTCA
AGTGTCAATTCCTGGAGATCAGATCACATTAGAATACATAAATAATCCACACCTGGTATCACGATTGG
CTCTTCTGCCATCCCAGCTAGGAGGTCTAAACTACCTGTCATGCAGTAGGCTGTTCAATCGAAACATAG
GCGACCCGGTGGTTTCCGCAGTTGCAGATCTTAAGAGATTAATTAAATCAGGATGTATGGATTACTGG
ATCCTTTATAACTTATTAGGGAGAAAACCGGGAAACGGCTCATGGGCTACTTTAGCAGCTGACCCGTA
CTCAATCAATATAGAGTATCAATACCCCCCAACTACAGCTCTTAAGAGGCACACCCAACAAGCTCTGA
TGGAACTCAGTACGAATCCAATGTTACGTGGCATATTCTCTGACAATGCACAGGCAGAAGAAAATAAT
CTTGCTAGATTTCTCCTGGATAGGGAGGTGATCTTTCCGCGTGTAGCTCACATCATCATTGAGCAAACC
AGTGTCGGGAGGAGAAAACAGATTCAAGGATATTTGGATTCAACTAGATCGATAATGAGTAAATCACT
AGAAATTAAGCCCTTGTCCAATAGGAAGCTTAATGAAATACTGGATTACAACATCAATTACCTAGCTT
ACAATTTGGCATTACTCAAGAATGCTATTGAACCTCCGACTTATTTGAAAGCAATGACTCTTGAAACAT
GTAGCATCGACATTGCAAGGAGCCTCCGGAAGCTCTCCTGGGCCCCACTCTTGGGTGGGAGAAATCTT
GAAGGATTAGAGACGCCAGATCCCATTGAAATTACTGCAGGAGCATTAATTGTTGGATCGGGCTACTG
TGAACAGTGTGCTGCAGGAGACAATCGATTCACATGGTTTTTCTTGCCATCTGGTATCGAGATAGGAG
GGGATCCCCGTGATAATCCTCCTATCCGTGTACCGTACATTGGCTCCAGGACTGATGAGAGGAGGGTA
GCCTCAATGGCATACATCAGGGGTGCCTCGAGTAGCCTAAAAGCAGTTCTTAGACTGGCGGGAGTGTA
CATCTGGGCATTCGGAGATACTCTGGAGAATTGGATAGATGCACTGGATTTGTCTCACACTAGAGTTA
ACATCACACTTGAACAGCTGCAATCCCTCACCCCACTTCCAACCTCTGCCAATCTAACCCATCGGTTGG
ATGATGGCACAACTACCCTAAAGTTTACTCCTGCGAGCTCTTACACCTTTTCAAGTTTCACTCATATAT
CAAATGATGAGCAATACCTGACAATTAATGACAAAACTGCAGATTCAAATATAATCTACCAACAGTTA
ATGATCACTGGACTCGGAATTTTAGAAACATGGAATAATCCCCCAATCAATAGAACATTCGAAGAATC
TACCCTACATTTGCACACTGGTGCATCATGTTGTGTCCGACCTGTGGACTCCTGCATCATCTCAGAAGC
ATTAACAGTCAAGCCACATATTACAGTACCGTACAGCAATAAATTTGTATTTGATGAAGACCCGCTAT
CTGAATATGAGACTGCAAAACTGGAATCGTTATCATTTCAAGCCCAATTAGGCAACATTGATGCTGTA
GATATGACAGGTAAATTAACATTATTGTCCCAATTCACTGCAAGGCAGATTATTAATGCAATCACTGG
ACTCGATGAGTCTGTCTCTCTTACTAATGATGCCATTGTTGCATCAGACTATGTCTCCAATTGGATTAG
TGAATGCATGTATACCAAATTAGATGAATTATTTATGTATTGTGGGTGGGAACTACTATTGGAACTATC
CTATCAAATGTATTATCTGAGGGTAGTTGGGTGGAGTAATATAGTGGATTATTCTTACATGATCTTGAG
AAGAATCCCGGGTGCAGCATTAAACAATCTGGCATCTACATTAAGTCATCCAAAACTTTTCCGACGAG
CTATCAACCTAGATATAGTTGCCCCCTTAAATGCTCCTCATTTTGCATCTCTGGACTACATCAAGATGA
GTGTGGATGCAATACTCTGGGGCTGTAAAAGAGTCATCAATGTGCTCTCCAATGGAGGGGACTTAGAA
TTAGTTGTGACATCTGAAGATAGCCTTATTCTCAGTGACCGATCCATGAATCTCATTGCAAGGAAATTA
ACTTTATTATCACTGATTCACCATAATGGTTTGGAACTACCAAAGATTAAGGGGTTCTCTCCTGATGAG
AAGTGTTTCGCTTTGACAGAATTTTTGAGGAAAGTGGTGAACTCAGGGTTGAGTTCAATAGAGAACCT
ATCAAATTTTATGTACAATGTGGAGAACCCACGGCTTGCAGCATTCGCCAGCAACAATTACTACCTGA
CCAGAAAATTATTGAATTCAATACGAGATACTGAGTCGGGTCAAGTAGCAGTCACCTCATATTATGAA
TCATTAGAATATATTGATAGTCTTAAGCTAACCCCACATGTGCCTGGTACCTCATGCATTGAGGATGAT
AGTCTATGTACAAATGATTACATAATCTGGATCATAGAGTCTAATGCAAACTTGGAGAAGTATCCAAT
TCCAAATAGCCCTGAGGATGATTCCAATTTCCATAACTTTAAGTTGAATGCTCCATCGCACCATACCTT
ACGCCCATTAGGGTTGTCATCAACTGCTTGGTATAAGGGTATAAGCTGTTGCAGGTACCTTGAGCGATT
AAAGCTACCACAAGGTGATCATTTATATATTGCAGAAGGTAGTGGTGCCAGTATGACAATCATAGAAT
ACCTATTCCCAGGAAGAAAGATATATTACAATTCTTTATTTAGTAGTGGTGACAATCCCCCACAAAGA
AATTATGCACCAATGCCTACTCAGTTCATTGAGAGTGTCCCATACAAGCTCTGGCAAGCACACACAGA
TCAATATCCCGAGATTTTTGAGGACTTCATCCCTCTATGGAACGGAAATGCCGCCATGACTGACATAG
GAATGACAGCTTGTGTAGAATTCATCATCAATCGAGTCGGCCCAAGGACTTGCAGTTTAGTACATGTA
GATTTGGAATCAAGTGCAAGCTTAAATCAACAATGCCTGTCAAAGCCGATAATTAATGCTATCATCAC
TGCTACAACTGTTTTGTGCCCTCATGGGGTGCTTATTCTGAAATATAGTTGGTTGCCATTTACTAGATTT
AGTACTTTGATCACTTTCTTATGGTGCTACTTTGAGAGAATCACTGTTCTTAGGAGCACATATTCTGGT
CCAGCTAATCATGAGGTTTATTTAATTTGTATCCTTGCCAACAACTTTGCATTCCAGACTGTCTCGCAG
GCAACAGGAATGGCGATGACTTTAACCGATCAAGGGTTTACTTTGATATCACCTGAAAGAATAAATCA
GTATTGGGATGGTCACTTGAAGCAAGAACGTATCGTAGCAGAAGCAATTGATAAGGTGGTTCTAGGAG
AAGATGCTCTATTCAATTCGAGTGATAATGAATTAATTCTCAAATGTGGAGGGACACCAAATGCACGG
AATCTTATCGATATCGAGCCAGTCGCAACTTTCATAGAATTTGAACAACTGATCTGCACAATGTTGACA
ACCCACTTGAAGGAAATAATTGATATAACAAGGTCTGGAACCCAGGATTATGAAAGTTTATTACTCAC
TCCTTACAATTTAGGTCTTCTTGGTAAAATCAGTACGATAGTGAGATTATTAACAGAAAGGATTCTAAA
TCATACTATCAGGAATTGGTTGATCCTCCCACCTTCGCTCCGGATGATCGTGAAGCAGGACTTGGAATT
CGGCATATTCAGGATTACTTCCATCCTCAATTCTGATCGGTTCCTGAAGCTTTCTCCAAATAGGAAATA
CTTGATTACACAATTAACTGCAGGCTACATTAGGAAATTGATTGAGGGGGATTGTAATATCGATCTAA
CCAGACCTATCCAAAAACAAATCTGGAAAGCATTAGGTTGTGTAGTCTATTGTCACGATCCAGTAGAT
CAAAGGGAATCAACAGAGTTTATTGATATAAATATTAATGAAGAAATAGACCGCGGGATCGATGGCG
AGGAAATCTAAATATATCAAGAATCAGAATTAGTTTAAGAAAAAAGAAGAGGATTAATCTTGGTTTTC
CCCTTGGT.

16. CVL67

The nucleic acid sequence for CVL67 is:

(SEQ ID NO: 75)
ACCAGGGGGAAAACGAAGTGGTGACTCAAATCATCGAAGACCCTCGAGA
TTACATAGGTCCGGAACCTATGGCCTTCGTGACCGACCTCGAGTCAGAG
TAGTTCAATAAGGACCTATCAAGTTTGGGCAATTTTTCGTCCCCGACAC
AAAAATGTCATCCGTGCTTAAAGCATATGAGCGATTCACACTCACTCAA
GAACTGCAAGATCAGAGTGAGGAAGGTACAATCCCACCTACAACACTAA
AACCGGTAATCAGGGTATTTATACTAACCTCTAATAACCCAGAGCTAAG
ATCCCGGCTTCTTCTATTCTGCCTACGGATTGTTCTCAGTAATGGTGCA
AGGGATTCCCATCGCTTTGGAGCATTACTTACAATGTTTTCGCTACCAT
CAGCCACAATGCTCAATCATGTCAAATTAGCTGACCAGTCACCAGAAGC
TGATATCGAAAGGGTAGAGATCGATGGCTTTGAGGAGGGATCATTCCGC
TTAATCCCCAATGCTCGTTCAGGTATGAGCCGTGGAGAGATCAATGCCT
ATGCTGCACTTGCAGAAGATCTACCTGACACACTAAACCATGCAACACC
TTTTGTTGATTCCGAAGTCGAGGGAACTGCATGGGATGAGATTGAGACT
TTCTTAGATATGTGTTACAGTGTCCTAATGCAGGCATGGATAGTGACTT
GCAAGTGCATGACTGCGCCAGACCAACCTGCTGCTTCTATTGAGAAACG
CCTGCAAAAATATCGTCAGCAAGGCAGGATCAACCCGAGATATCTCCTG
CAACCGGAGGCTCGAAGAATAATCCAGAATGTAATCCGAAAGGGAATGG
TGGTCAGACATTTCCTCACCTTTGAACTGCAGCTTGCCCGAGCACAAAG
CCTTGTATCAAATAGGTATTATGCTATGGTAGGGGATGTTGGAAAGTAT
ATAGAGAATTGTGGAATGGGAGGCTTCTTTTTGACACTAAAATATGCAT
TAGGAACCAGATGGCCCACACTTGCTTTAGCTGCATTTTCAGGAGAGCT
AACAAAGCTAAAGTCCCTCATGGCATTATACCAGACCCTTGGTGAGCAG
GCCCGATATTTGGCCCTATTGGAGTCACCACATTTGATGGATTTTGCTG
CAGCAAACTACCCACTGCTATATAGCTATGCTATGGGAATAGGCTATGT
GTTAGATGTCAACATGAGGAACTACGCTTTCTCCAGATCATACATGAAT
AAGACATATTTCCAATTGGGAATGGAAACTGCAAGAAAACAACAGGGTG
CAGTTGACATGAGGATGGCAGAAGATCTCGGTCTAACTCAAGCCGAACG
CACCGAGATGGCAAATACACTTGCCAAATTGACCACAGCAAATCGAGGG
GCAGACACCAGGGGAGGAGTCAACCCGTTCTCATCTATCACTGGGACAA
CTCAGGTGCCCGCTGCAGCAACAGGTGACACATTCGAGAGTTACATGGC
AGCGGATCGACTGAGGCAGAGATATGCTGATGCAGGCACCCACGATGAT
GAGATGCCACCATTGGAAGAGGAGGAAGAGGACGACACATCTGCAGGTC
CACGCACTGGACTAACTCTTGAACAAGTGGCCTTGGACATCCAGAACGC
AGCAGTTGGAGCTCCCATCCATACAGATGACCTGAATGCCGCACTGGGT
GATCTTGACATCTAGACAATTCAGATCCCAATCTTAAATCGACACACCT
AATTGACCAGTTAGATGGAACTACAGTGGATTCCATAAGGTTCCTGCCT
ACCATCGGCTTTTAAGAAAAAAATAGGCCCGGACGGGTTAGCAACAAGC
GACTGCCGATGCCAATAACACAATCCACAATCTACAATGGATCCCACTG
ATCTGAGCTTCTCCCCAGATGAGATCAATAAGCTCATAGAGACAGGCCT
GAATACTGTGGAGTATTTTACTTCCCAACAAGTCACAGGAACATCCTCT
CTTGGAAAGAATACAATACCACCAGGGGTCACAGGACTACTAACCAATG
CTGCAGAGGCAAAGATCCAAGAGTCAACCAACCATCAGAAGGGTTCAGT
TGGTGGGGGCGCAAAACCAAAGAAACCGCGACCAAAAATTGCCATTGTG
CCAGCAGATGACAAAACGGTGCCCGGAAAGCCGATCCCAAACCCTCTAT
TAGGTCTGGACTCCACCCCGAGCACCCAAACTGTGCTTGATCTAAGTGG
GAAAACATTACCATCAGGATCCTATAAGGGGGTTAAACTTGCGAAATTT
GGAAAAGAAAATCTGATGACACGGTTCATCGAGGAACCCAGAGAGAATC
CTATCGCAACCAATTCCCCCATCGATTTTAAGAGGGGCAGGGATACCGG
CGGGTTCCATAGAAGGGAGTACTCAATCGGATGGGTGGGAGATGAAGTC
AAGGTCACTGAGTGGTGCAATCCATCCTGTTCTCCAATCACCGCTGCAG
CAAGGCGATTTGAATGCACTTGTCACCAGTGTCCAGTCACTTGCTCTGA
ATGTGAACGAGATACTTAATACAGTGAGAAATTTGGACTCTCGGATGAA
TCAACTGGAGACAAAAGTAGATCGCATTCTCTCATCTCAGTCTCTAATC
CAGACCATCAAGAATGACATAGTTGGACTTAAAGCAGGGATGGCTACTT
TAGAAGGAATGATTACAACTGTGAAAATCATGGACCCGGGAGTTCCCAG
TAATGTTACTGTGGAAGATGTACGCAAGAAACTAAGTAACCATGCTGTT
GTTGTGCCAGAATCATTCAATGATAGTTTCTTGACTCAATCTGAAGATG
TAATTTCACTTGATGAGTTGGCTCGACCAACTGCAACAAGTGTTAAGAA
GATTGTCAGGAAGGTTCCTCCTCAGAAGGATCTGACTGGATTGAAGATC
ACACTAGAGCAATTGGCAAAGGATTGCATCAGCAAACCGAAGATGAGGG
AAGAGTATCTCCTCAAAATCAACCAGGCTTCCAGTGAGGCTCAGCTAAT
TGACCTCAAGAAAGCAATCATCCGCAGTGCAATTTGATCAAGAAACACC
CAATTACACTACACTGGTATGACACTGTACTAACCCTGAGGGTTTTAGA
AAAAACGATTAACGATAAATAAGCCCGAACACTACACACCACCCGAGGC
AGCCATGCCATCCATCAGCATCCCCGCAGACCCCACCAATCCACGTCAA
TCAATAAAAGCGTTCCCAATTGTGATCAACCGTGATGGGGGTGAGAAAG
GTCGCTTGGTTAAACAACTACGCACAACCTACTTGAATGACCTAGATAC
TCATGAGCCACTGGTGACATTCGTAAATACTTATGGATTCATCTACGAA
CAGGATCGGGGAAATACCATTGTCGGAGAGGATCAACATGGGAAGAAAA
GAGAGGCTGTGACTGCTGCAATGGTTACCCTTGGATGTGGGCCTAATCT
ACCATCATTAGGGAATGTCCTGGGACAACTGAGTGAATTCCAGGTCATT
GTTAGGAAGACATCCAGCAAAGCGGAAGAGATGGTCTTTGAAATTGTTA
AGTATCCGAGAATATTTCGGGGTCATACATTAATCCAGAAAGGACTAGT
CTGTGTCTCCGCAGAAAAATTTGTTAAGTCACCAGGGAAAGTACAATCT
GGAATGGACTATCTCTTCATTCCGACATTTCTGTCAGTGACTTACTGTC
CAGCTGCAATCAAATTTCAGGTACCTGGCCCCATGTTGAAAATGAGATC
AAGATACACTCAGAGCTTACAACTTGAACTAATGATAAGAATCCTGTGT
AAGCCCGATTCGCCACTTATGAAGGTCCATATCCCTGACAAGGAAGGAA
GAGGATGTCTTGTATCAGTATGGTTGCATGTATGCAATATCTTCAAATC
AGGAAACAAGAATGGCAGTGAGTGGCAGGAATACTGGATGAGAAAGTGT
GCTAACATGCAACTTGAAGTGTCGATTGCAGATATGTGGGGACCAACTA
TCATAATTCATGCCAGAGGTCACATTCCCAAAAGTGCTAAGTTGTTTTT
TGGAAAGGGTGGATGGAGCTGCCATCCACTTCACGAAGTTGTTCCAAGT
GTCACTAAAACACTATGGTCCGTGGGCTGTGAGATTACAAAGGCGAAGG
CAATAATACAAGAGAGTAGCATCTCTCTTCTCGTGGAGACTACTGACAT
CATAAGTCCAAAAGTCAAAATTTCATCTAAGCATCGCCGCTTTGGGAAA
TCAAATTGGGGTCTGTTCAAGAAAACCAAATCACTGCCCAACCTGACGG
AGCTGGAATGACTGACCCCCAATCGAGACTACACCACCTCAAACTATAG
GTGGGTGGTACCTCAGTGATTAATCTCGTAAGCACTGATCGTAGGCTAC
AACACACTAATATTATCCAGATTAGAGAGCTTAATTAGCTCTGTATTAA
TAATAACACTACTATTCCAATAACTGGAATCACCAGCTTGATTTATCTC
CAAAATGATTCAAAGAAAACAAATCATATTAAGACTATCCTAAGCACGA
ACCCATATCGTCCTTCAAATCATGGGTACTATAATTCAATTTCTGGTGG
TCTCCTGTCTATTGGCAGGAGCAGGCAGTCTTGATCCAGCAGCCCTCAT
GCAAATCGGTGTCATTCCAACAAATGTCCGGCAACTTATGTATTATACT
GAGGCCTCATCAGCATTCATTGTTGTGAAGTTAATGCCTACAATTGACT
CGCCGATTAGTGGATGTAATATAACATCAATTTCAAGCTATAATGCAAC
AGTGACAAAACTCCTACAGCCGATCGGTGAGAATTTGGAGACGATTAGG
AACCAGTTGATTCCAACTCGGAGGAGACGCCGGTTTGCAGGGGTGGTGA
TTGGATTAGCTGCATTAGGAGTAGCTACTGCCGCACAGGTCACTGCCGC
AGTAGCACTAGTAAAGGCAAATGAAAATGCTGCGGCTATACTCAATCTC
AAAAATGCAATCCAAAAAACAAATGCAGCAGTTGCAGATGTGGTCCAGG
CCACACAATCACTAGGAACaGCAGTTCAAGCAGTTCAAGATCACATAAA
CAGTGTGGTAAGTCCAGCAATTACAGCAGCCAATTGTAAAGCCCAAGAT
GCTATCATTGGCTCAATCCTCAATCTCTATTTGACCGAGTTGACAACTA
TCTTCCACAATCAAATTACAAACCCTGCATTGAGTCCTATTACAATTCA
AGCTTTAAGGATCCTACTAGGGAGTACCTTGCCGACTGTGGTCGAAAAA
TCTTTCAATACCCAGATAAGTGCGGCTGAGCTTCTCTCATCAGGGTTAT
TGACAGGCCAGATTGTGGGATTAGATTTGACCTATATGCAGATGGTCAT
AAAAATTGAGCTGCCAACTTTAACTGTACAACCTGCAACCCAGATCATA
GATCTGGCCACCATTTCTGCATTCATTAACAATCAAGAAGTCATGGCCC
AATTACCAACACGTGTTATTGTGACTGGCAGCTTGATCCAAGCCTATCC
CGCATCGCAATGCACTATTACACCCAACACTGTGTACTGTAGGTATAAT
GATGCCCAAGTACTCTCAGATGATACGATGGCTTGCCTCCAAGGTAACT
TGACAAGATGCACCTTCTCTCCAGTGGTTGGGAGCTTTCTCACTCGATT
CGTGCTGTTCGATGGAATAGTTTATGCAAATTGCAGGTCGATGTTGTGC
AAGTGCATGCAGCCTGCTGCTGTGATCCTACAGCCGAGTTCATCCCCTG
TAACTGTCATTGACATGTACAAATGTGTGAGTCTGCAGCTTGACAATCT
CAGATTCACCATCACTCAATTGGCCAATGTAACCTACAATAGCACCATC
AAGCTTGAAACATCCCAGATCTTGCCTATTGATCCGTTGGATATATCCC
AGAATCTAGCTGCGGTGAATAAGAGTCTAAGTGATGCACTACAACACTT
AGCACAAAGTGACACATACCTTTCTGCAATCACATCAGCTACGACTACA
AGTGTATTATCCATAATAGCAATCTGTCTTGGATCGTTAGGTTTAATAT
TAATAATCTTGCTCAGTGTAGTTGTGTGGAAGTTATTGACCATTGTCGC
TGCTAATCGAAATAGAATGGAGAATTTTGTTTATCATAATTCAGCATTC
CACCACTCACGATCTGATCTCAGTGAGAAAAATCAACCTGCAACTCTTG
GAACAAGATAAGACAGTCATCCATTAGTAATTTTTAAGAAAAAAACGAT
AGGACCGAAACTAGTATTGAAAGAACCGTCTCGGTCAATCTAGGTAATC
GAGCTGcTACCGTCTCGGAAAGCTCAAATCATGCTGCCTGATCCGGAAG
ATCCGGAAAGCAAAAAAGCTACAAGGAGAACAGGAAACCTAATTATCTG
CTTCCTATTCATCTTCTTTCCGTTTGTAAACTTCATTGTTCCAACTCTA
AGACACTTGCTGTCCTAACACCTGCTATAGGCTATCCACTGCATCATCT
CTTTTTAAGAAAAAAATAGGCCCGGACGGGTTAGCAACAAGCGACTGCC
GGTGCCAACAACACAATCCACAATCTACAGTCGACGCGGCCGCCatgtt
cgtcttcctggtcctgctgcctctggtctcctcacagtgcgtcaatctg
acaactcggactcagctgccacctgcttatactaatagcttcaccagag
gcgtgtactatcctgacaaggtgtttagaagctccgtgctgcactctac
acaggatctgtttctgccattctttagcaacgtgacctggttccacgcc
atccacgtgagcggcaccaatggcacaaagcggttcgacaatcccgtgc
tgccttttaacgatggcgtgtacttcgcctctatcgagaagagcaacat
catcagaggctggatctttggcaccacactggactccaagacacagtct
ctgctgatcgtgaacaatgccaccaacgtggtcatcaaggtgtgcgagt
tccagttttgtaatgatcccttcctgggcgtgacatccaaccacaagaa
caataagagctggatggagtccgagtttagagtgtattctagcgccaac
aactgcacatttgagtacgtgagccagcctttcctgatggacctggagg
gcaagcagggcaatttcaagaacctgagggagttcgtgtttaagaatat
cgacggctacttcaaaatctactctaagcacacccccatcaacctggtg
cgcgacctgcctcagggcttcagcgccctggagcccctggtggatctgc
ctatcggcatcaacatcacccggtttcagacactgctggccctgcacag
aagctacctgacacccggcgactcctctagcggatggaccgccggcgct
gccgcctactatgtgggctacctccagccccggaccttcctgctgaagt
acaacgagaatggcaccatcacagacgcagtggattgcgccctggaccc
cctgagcgagacaaagtgtacactgaagtcctttaccgtggagaagggc
atctatcagacatccaatttcagggtgcagccaaccgagtctatcgtgc
gctttcctaatatcacaaacctgtgcccatttggcgaggtgttcaacgc
aaccaagttcgccagcgtgtacgcctggaataggaagcggatcagcaac
tgcgtggccgactatagcgtgctgtacaactccgcctctttcagcacct
ttaagtgctatggcgtgtcccccacaaagctgaatgacctgtgctttac
caacgtctacgccgattctttcgtgatcaggggcgacgaggtgcgccag
atcgcccccggccagacaggcaagatcgcagactacaattataagctgc
cagacgatttcaccggctgcgtgatcgcctggaacagcaacaatctgga
ttccaaagtgggcggcaactacaattatctgtaccggctgtttagaaag
agcaatctgaagcccttcgagagggacatctctacagaaatctaccagg
ccggcagcaccccttgcaatggcgtgaagggcttcaactgctacttccc
tctgcaatcctacggctttcagcccacatacggcgtgggctaccagcca
taccgggtcgtggtgctgagcttcgagctgctgcacgcccctgccacag
tgtgcggccctaagaagagcaccaatctggtaaagaacaagtgcgtcaa
cttcaatttcaacggcctgaccgggaccggagtgctgaccgaaagcaac
aagaaattcctgccgttccagcagttcggcagagatatcgccgacacca
ccgatgccgtgcgggacccccagacacttgaaatcctggacatcacccc
ttgcagttttggcggcgtgtccgtgatcacacctggaacaaacaccagc
aaccaggtggccgtgctgtaccagggcgtcaactgcaccgaggtccccg
tggccatccacgccgatcagctgacacctacatggcgggtgtactctac
cggcagcaacgtgttccaaaccagagccggctgcctgatcggcgctgag
cacgtgaacaacagctacgagtgcgacattcctatcggtgctggcatct
gcgcctcttaccagacacagacaaacagccacagaagagcccggagcgt
ggcctcccagtctatcatcgcctataccatgtccctgggcgccgagaac
agcgtggcctactctaacaatagcatcgccatcccaaccaacttcacaa
tctctgtgaccacagagatcctgcccgtgtccatgaccaagacatctgt
ggactgcacaatgtatatctgtggcgattctaccgagtgcagcaacctg
ctgctccagtacggcagcttttgtacccagctgaatagagccctgacag
gcatcgccgtggagcaggataagaacacacaggaggtgttcgcccaggt
gaagcaaatctacaagaccccccctatcaaggactttggcggcttcaat
ttttcccagatcctgcctgatccatccaagccttctaagcggagcttta
tcgaggacctgctgttcaacaaggtgaccctggccgatgccggcttcat
caagcagtatggcgattgcctgggcgacatcgcagccagggacctgatc
tgcgcccagaagtttaatggcctgaccgtgctgccacccctgctgacag
atgagatgatcgcacagtacacaagcgccctgctggccggcaccatcac
atccggatggaccttcggcgcaggagccgccctccagatcccctttgcc
atgcagatggcctataggttcaacggcatcggcgtgacccagaatgtgc
tgtacgagaaccagaagctgatcgccaatcagtttaactccgccatcgg
caagatccaggacagcctgtcctctacagccagcgccctgggcaagctc
cagaatgtggtgaatcagaacgcccaggccctgaataccctggtgaagc
agctgagcagcaacttcggcgccatctctagcgtgctgaatgacatcct
gagccggctggacaaggtggaggcagaggtgcagatcgaccggctgatc
accggccggctccagagcctccagacctatgtgacacagcagctgatca
gggccgccgagatcagggccagcgccaatctggcagcaaccaagatgtc
cgagtgcgtgctgggccagtctaagagagtggacttttgtggcaagggc
tatcacctgatgtccttccctcagtctgccccacacggcgtggtgtttc
tgcacgtgacctacgtgcccgcccaggagaagaacttcaccacagcccc
tgccatctgccacgatggcaaggcccactttccaagggagggcgtgttc
gtgtccaacggcacccactggtttgtgacacagcgcaatttctacgagc
cccagatcatcaccacagacaacaccttcgtgagcggcaactgtgacgt
ggtcatcggcatcgtgaacaataccgtgtatgatccactccagcccgag
ctggacagctttaaggaggagctggataagtatttcaagaatcacacct
cccctgacgtggatctgggcgacatcagcggcatcaatgcctccgtggt
gaacatccagaaggagatcgaccgcctgaacgaggtggctaagaatctg
aacgagagcctgatcgacctccaggagctgggcaagtatgagcagtaca
tcaagtggccctggtacatctggctgggcttcatcgccggcctgatcgc
catcgtgatggtgaccatcatgctgtgctgtatgacatcctgctgttct
tgcctgaagggctgctgtagctgtggctcctgctgtAAGTTATTGACCA
TTGTCGTTGCTAATCGAAATAGAATGGAGAATTTTGTTTATCATAAATG
ATGAACGCGTGCTAGCCTCCTGCCATACTTCCTACTCACATCATATCTA
TTTTAAAGAAAAAATAGGCCCGAACACTAATCGTGCCGGCAGTGCCACT
GCACACACAACACTACACATACAATACACTACAATGGTTGCAGAAGATG
CCCCTGTTAGGGGCACTTGCCGAGTATTATTTCGAACAACAACTTTAAT
TTTTCTATGCACACTATTAGCATTAAGCATCTCTATCCTTTATGAGAGT
TTAATAACCCAAAAGCAAATCATGAGCCAAGCAGGCTCAACTGGATCTA
ATTCTAGATTAGGAAGTATCACTGATCTTCTTAATAATATTCTCTCTGT
CGCAAATCAGATTATATATAACTCTGCAGTCGCTCTACCTCTACAATTG
GACACTCTTGAATCAACACTCCTTACAGCCATTAAGTCTCTTCAAACAA
GTGACAAGCTAGAACAGAACTGCTCGTGGGGTGCTGCACTGATTAATGA
TAATAGATACATTAATGGCATCAATCAGTTCTATTTCTCAATTGCTGAG
GGTCGCAAGCTGACACTTGGCCCACTTCTTAATATACCTAGTTTCATTC
CAACTGCCACGACACCAGAGGGCTGCACCAGGATCCCATCATTCTCGCT
CACCAAGACACACTGGTGTTATACACACAATGTTATCCTGAATGGATGC
CAGGATCATGTATCCTCAAATCAATTTGTTTCCATGGGAATCATTGAAC
CCACTTCTGCCGGGTTTCCATCCTTTCGAACCCTAAAGACTCTATATCT
CAGCGATGGGGTCAATCGTAAGAGCTGCTCTATCAGTACAGTTCCGGGG
GGTTGTATGATGTACTGTTTCGTCTCTACTCAACCAGAGAGGGATGACT
ACTTTTCTACCGCTCCTCCAGAACAACGAATTATTATAATGTACTATAA
TGATACAATCGTGGAGCGCATAATTAATCCACCCGGGGTACTAGATGTA
TGGGCAACATTGAACCCAGGAACAGGAAGCGGGGTATATTATTTAGGTT
GGGTACTCTTTCCAATATATGGCGGCGTGATTAAAGATACGAGTTTATG
GAATAATCAAGCAAATAAATACTTTATCCCCCAGATGGTTGCTGCTCTC
TGCTCACAAAACCAGGCAACTCAAGTCCAAAATGCTAAGTCATCATACT
ATAGCAGCTGGTTTGGCAATCGAATGATTCAGTCTGGGATCCTGGCATG
TCCTCTTCAACAGGATCTAACCAATGAGTGTTTAGTTCTGCCCTTTTCT
AATGATCAGGTGCTTATGGGTGCTGAAGGGAGATTATACATGTATGGTG
ACTCGGTGTATTACTACCAAAGAAGCAATAGTTGGTGGCCTATGACCAT
GCTGTATAAGGTAACCATAACATTCACTAATGGTCAGCCATCTGCTATA
TCAGCTCAGAATGTGCCCACACAGCAGGTCCCTAGACCTGGGACAGGAA
GCTGCTCTGCAACAAATAGATGTCCCGGTTTTTGCTTGAAAGGAGTGTA
TGCTGATGCCTGGTTACTGACCAACCCTTCGTCTACCAGTACATTTGGA
TCAGAAGCAACCTTCACTGGTTCTTATCTCAACGCAGCAACTCAGCGTA
TCAATCCGACGATGTATATCGCGAACAACACACAGATCATAAGCTCACA
GCAATTTGGATCAAGCGGTCAAGAAGCAGCATATGGCCACACAACTTGT
TTTAGGGACACAGGCTCTGTTATGGTATACTGTATCTATATTATTGAAT
TGTCCTCATCTCTCTTAGGACAATTTCAGATTGTCCCATTTATCCGTCA
GGTGACACTATCCTAAAGGCAGAAGCCTCCAGGTCTGACCCAGCCAATC
AAAGCATTATACCAGACCATGGAATGCATACCAAACATTATTGACACTA
ATGACACACAAAATTGGTTTTAAGAAAAACCAAGAGAACAATAGGCCAG
AATGGCTGGGTCTCGGGAGATATTACTCCCTGAAGTCCATCTCAATTCA
CCAATTGTAAAGCATAAGCTATACTATTACATTCTACTTGGAAACCTCC
CAAATGAGATCGACATTGACGATTTAGGTCCATTACATAATCAAAATTG
GAATCAAATAGCACATGAAGAGTCTAACTTAGCCCAACGCTTGGTAAAT
GTAAGAAATTTTCTAATTACCCACATCCCTGATCTTAGAAAGGGCCATT
GGCAAGAGTATGTCAATGTAATACTGTGGCCGCGAATTCTTCCCTTGAT
CCCGGATTTTAAAATCAATGACCAATTGCCTCTACTCAAAAATTGGGAC
AAGTTAGTTAAAGAATCATGTTCAGTAATCAATGCGGGTACTTCCCAGT
GCATTCAGAATCTCAGCTATGGACTGACAGGTCGTGGGAACCTCTTTAC
ACGATCACGTGAACTCTCTGGTGACCGCAGGGATATTGATCTTAAGACG
GTTGTGGCAGCATGGCATGACTCAGACTGGAAAAGAATAAGTGATTTTT
GGATTATGATCAAATTCCAGATGAGACAATTAATTGTTAGGCAAACAGA
TCATAATGATCCTGATTTAATCACGTATATCGAAAATAGAGAAGGCATA
ATCATCATAACCCCTGAACTGGTAGCATTATTTAACACTGAGAATCATA
CACTAACATACATGACCTTTGAAATTGTACTGATGGTTTCAGATATGTA
CGAAGGTCGTCACAACATTTTATCACTATGCACAGTTAGCACTTACCTG
AATCCTCTGAAGAAAAGAATAACATATTTATTGAGCCTTGTAGATAACT
TAGCTTTTCAGATAGGTGATGCTGTATATAACATAATTGCTTTGCTAGA
ATCCTTTGTATATGCACAGTTGCAAATGTCAGATCCCATCCCAGAACTC
AGAGGACAATTCCATGCATTCGTATGTTCTGAGATTCTTGATGCACTAA
GGGGAACTAATAGTTTCACCCAGGATGAATCAAGAACTGTGACAACCAA
TTTGATATCCCCATTCCAAGATCTGACCCCAGATCTTACGGCTGAATTG
CTCTGTATAATGAGGCTTTGGGGACACCCCATGCTCACCGCCAGTCAAG
CTGCGGGAAAGGTACGCGAGTCCATGTGTGCTGGAAAAGTATTAGACTT
TCCCACCATTATGAAAACACTAGCCTTTTTCCATACTATTCTGATCAAT
GGATACAGGAGGAAGCATCATGGAGTATGGCCACCCTTAAACTTACCGG
GTAATGCTTCAAAGGGTCTCACGGAACTTATGAATGACAATACTGAGAT
AAGCTATGAATTCACACTTAAGCATTGGAAGGAAATCTCTCTTATAAAA
TTCAAGAAATGTTTTGATGCAGACGCAGGTGAGGAACTCAGTATATTTA
TGAAAGATAAAGCAATTAGTGCCCCAAAACAAGATTGGATGAGTGTGTT
TAGAAGAAGCCTAATCAAACAGCGCCATCAGCATCATCAGGTCCCCCTA
CCAAATCCATTCAATCGACGGCTATTGCTAAACTTTCTCGGAGATGACA
AATTCGACCCGAATGTGGAGCTACAGTATGTAACATCAGGTGAGTATCT
ACATGATGACACGTTTTGTGCATCATATTCACTAAAAGAGAAGGAAATT
AAACCTGATGGTCGAATTTTTGCAAAGTTGACTAAGAGAATGAGATCAT
GTCAAGTTATAGCAGAATCTCTTTTAGCGAACCATGCTGGGAAGTTAAT
GAAAGAGAATGGTGTTGTGATGAATCAGCTATCATTAACAAAATCACTA
TTAACAATGAGTCAGATTGGAATAATATCCGAGAAAGCTAGAAAATCGA
CTCGAGATAACATAAATCAACCTGGTTTCCAGAATATCCAGAGAAATAA
ATCACATCACTCCAAGCAAGTCAATCAGCGAGATCCAAGTGATGACTTT
GAATTGGCAGCATCTTTTTTAACTACTGATCTCAAAAAATATTGTTTAC
AATGGAGGTACCAGACAATTATCCCATTTGCTCAATCATTAAACAGAAT
GTATGGTTATCCTCATCTCTTTGAGTGGATTCACTTACGGCTAATGCGT
AGTACACTTTACGTGGGGGATCCCTTCAACCCACCAGCAGATACCAGTC
AATTTGATCTAGATAAAGTAATTAATGGAGATATCTTCATTGTATCACC
CAGAGGTGGAATTGAAGGGCTATGTCAAAAGGCTTGGACAATGATATCT
ATCTCTGTGATAATTCTATCTGCCACAGAGTCTGGCACACGAGTAATGA
GTATGGTGCAGGGAGATAATCAAGCAATTGCTGTCACCACACGAGTACC
AAGGAGCCTGCCGACTCTTGAGAAAAAGACTATTGCTTTTAGATCTTGT
AATCTATTCTTTGAGAGGTTAAAATGTAATAATTTTGGATTAGGTCACC
ATTTGAAAGAACAAGAGACTATCATTAGTTCTCACTTCTTTGTTTATAG
CAAGAGAATATTCTATCAGGGGAGGATTCTAACGCAAGCCTTAAAAAAT
GCTAGTAAGCTCTGCTTGACAGCTGATGTCCTAGGAGAATGTACCCAAT
CATCATGTTCTAATCTTGCAACTACTGTCATGAGGTTAACTGAGAATGG
TGTTGAAAAAGATATCTGTTTCTACTTGAATATCTATATGACCATCAAA
CAGCTCTCCTATGATATCATCTTCCCTCAAGTGTCAATTCCTGGAGATC
AGATCACATTAGAATACATAAATAATCCACACCTGGTATCACGATTGGC
TCTTCTGCCATCCCAGCTAGGAGGTCTAAACTACCTGTCATGCAGTAGG
CTGTTCAATCGAAACATAGGCGACCCGGTGGTTTCCGCAGTTGCAGATC
TTAAGAGATTAATTAAATCAGGATGTATGGATTACTGGATCCTTTATAA
CTTATTAGGGAGAAAACCGGGAAACGGCTCATGGGCTACTTTAGCAGCT
GACCCGTACTCAATCAATATAGAGTATCAATACCCCCCAACTACAGCTC
TTAAGAGGCACACCCAACAAGCTCTGATGGAACTCAGTACGAATCCAAT
GTTACGTGGCATATTCTCTGACAATGCACAGGCAGAAGAAAATAATCTT
GCTAGATTTCTCCTGGATAGGGAGGTGATCTTTCCGCGTGTAGCTCACA
TCATCATTGAGCAAACCAGTGTCGGGAGGAGAAAACAGATTCAAGGATA
TTTGGATTCAACTAGATCGATAATGAGTAAATCACTAGAAATTAAGCCC
TTGTCCAATAGGAAGCTTAATGAAATACTGGATTACAACATCAATTACC
TAGCTTACAATTTGGCATTACTCAAGAATGCTATTGAACCTCCGACTTA
TTTGAAAGCAATGACTCTTGAAACATGTAGCATCGACATTGCAAGGAGC
CTCCGGAAGCTCTCCTGGGCCCCACTCTTGGGTGGGAGAAATCTTGAAG
GATTAGAGACGCCAGATCCCATTGAAATTACTGCAGGAGCATTAATTGT
TGGATCGGGCTACTGTGAACAGTGTGCTGCAGGAGACAATCGATTCACA
TGGTTTTTCTTGCCATCTGGTATCGAGATAGGAGGGGATCCCCGTGATA
ATCCTCCTATCCGTGTACCGTACATTGGCTCCAGGACTGATGAGAGGAG
GGTAGCCTCAATGGCATACATCAGGGGTGCCTCGAGTAGCCTAAAAGCA
GTTCTTAGACTGGCGGGAGTGTACATCTGGGCATTCGGAGATACTCTGG
AGAATTGGATAGATGCACTGGATTTGTCTCACACTAGAGTTAACATCAC
ACTTGAACAGCTGCAATCCCTCACCCCACTTCCAACCTCTGCCAATCTA
ACCCATCGGTTGGATGATGGCACAACTACCCTAAAGTTTACTCCTGCGA
GCTCTTACACCTTTTCAAGTTTCACTCATATATCAAATGATGAGCAATA
CCTGACAATTAATGACAAAACTGCAGATTCAAATATAATCTACCAACAG
TTAATGATCACTGGACTCGGAATTTTAGAAACATGGAATAATCCCCCAA
TCAATAGAACATTCGAAGAATCTACCCTACATTTGCACACTGGTGCATC
ATGTTGTGTCCGACCTGTGGACTCCTGCATCATCTCAGAAGCATTAACA
GTCAAGCCACATATTACAGTACCGTACAGCAATAAATTTGTATTTGATG
AAGACCCGCTATCTGAATATGAGACTGCAAAACTGGAATCGTTATCATT
TCAAGCCCAATTAGGCAACATTGATGCTGTAGATATGACAGGTAAATTA
ACATTATTGTCCCAATTCACTGCAAGGCAGATTATTAATGCAATCACTG
GACTCGATGAGTCTGTCTCTCTTACTAATGATGCCATTGTTGCATCAGA
CTATGTCTCCAATTGGATTAGTGAATGCATGTATACCAAATTAGATGAA
TTATTTATGTATTGTGGGTGGGAACTACTATTGGAACTATCCTATCAAA
TGTATTATCTGAGGGTAGTTGGGTGGAGTAATATAGTGGATTATTCTTA
CATGATCTTGAGAAGAATCCCGGGTGCAGCATTAAACAATCTGGCATCT
ACATTAAGTCATCCAAAACTTTTCCGACGAGCTATCAACCTAGATATAG
TTGCCCCCTTAAATGCTCCTCATTTTGCATCTCTGGACTACATCAAGAT
GAGTGTGGATGCAATACTCTGGGGCTGTAAAAGAGTCATCAATGTGCTC
TCCAATGGAGGGGACTTAGAATTAGTTGTGACATCTGAAGATAGCCTTA
TTCTCAGTGACCGATCCATGAATCTCATTGCAAGGAAATTAACTTTATT
ATCACTGATTCACCATAATGGTTTGGAACTACCAAAGATTAAGGGGTTC
TCTCCTGATGAGAAGTGTTTCGCTTTGACAGAATTTTTGAGGAAAGTGG
TGAACTCAGGGTTGAGTTCAATAGAGAACCTATCAAATTTTATGTACAA
TGTGGAGAACCCACGGCTTGCAGCATTCGCCAGCAACAATTACTACCTG
ACCAGAAAATTATTGAATTCAATACGAGATACTGAGTCGGGTCAAGTAG
CAGTCACCTCATATTATGAATCATTAGAATATATTGATAGTCTTAAGCT
AACCCCACATGTGCCTGGTACCTCATGCATTGAGGATGATAGTCTATGT
ACAAATGATTACATAATCTGGATCATAGAGTCTAATGCAAACTTGGAGA
AGTATCCAATTCCAAATAGCCCTGAGGATGATTCCAATTTCCATAACTT
TAAGTTGAATGCTCCATCGCACCATACCTTACGCCCATTAGGGTTGTCA
TCAACTGCTTGGTATAAGGGTATAAGCTGTTGCAGGTACCTTGAGCGAT
TAAAGCTACCACAAGGTGATCATTTATATATTGCAGAAGGTAGTGGTGC
CAGTATGACAATCATAGAATACCTATTCCCAGGAAGAAAGATATATTAC
AATTCTTTATTTAGTAGTGGTGACAATCCCCCACAAAGAAATTATGCAC
CAATGCCTACTCAGTTCATTGAGAGTGTCCCATACAAGCTCTGGCAAGC
ACACACAGATCAATATCCCGAGATTTTTGAGGACTTCATCCCTCTATGG
AACGGAAATGCCGCCATGACTGACATAGGAATGACAGCTTGTGTAGAAT
TCATCATCAATCGAGTCGGCCCAAGGACTTGCAGTTTAGTACATGTAGA
TTTGGAATCAAGTGCAAGCTTAAATCAACAATGCCTGTCAAAGCCGATA
ATTAATGCTATCATCACTGCTACAACTGTTTTGTGCCCTCATGGGGTGC
TTATTCTGAAATATAGTTGGTTGCCATTTACTAGATTTAGTACTTTGAT
CACTTTCTTATGGTGCTACTTTGAGAGAATCACTGTTCTTAGGAGCACA
TATTCTGGTCCAGCTAATCATGAGGTTTATTTAATTTGTATCCTTGCCA
ACAACTTTGCATTCCAGACTGTCTCGCAGGCAACAGGAATGGCGATGAC
TTTAACCGATCAAGGGTTTACTTTGATATCACCTGAAAGAATAAATCAG
TATTGGGATGGTCACTTGAAGCAAGAACGTATCGTAGCAGAAGCAATTG
ATAAGGTGGTTCTAGGAGAAGATGCTCTATTCAATTCGAGTGATAATGA
ATTAATTCTCAAATGTGGAGGGACACCAAATGCACGGAATCTTATCGAT
ATCGAGCCAGTCGCAACTTTCATAGAATTTGAACAACTGATCTGCACAA
TGTTGACAACCCACTTGAAGGAAATAATTGATATAACAAGGTCTGGAAC
CCAGGATTATGAAAGTTTATTACTCACTCCTTACAATTTAGGTCTTCTT
GGTAAAATCAGTACGATAGTGAGATTATTAACAGAAAGGATTCTAAATC
ATACTATCAGGAATTGGTTGATCCTCCCACCTTCGCTCCGGATGATCGT
GAAGCAGGACTTGGAATTCGGCATATTCAGGATTACTTCCATCCTCAAT
TCTGATCGGTTCCTGAAGCTTTCTCCAAATAGGAAATACTTGATTACAC
AATTAACTGCAGGCTACATTAGGAAATTGATTGAGGGGGATTGTAATAT
CGATCTAACCAGACCTATCCAAAAACAAATCTGGAAAGCATTAGGTTGT
GTAGTCTATTGTCACGATCCAGTAGATCAAAGGGAATCAACAGAGTTTA
TTGATATAAATATTAATGAAGAAATAGACCGCGGGATCGATGGCGAGGA
AATCTAAATATATCAAGAATCAGAATTAGTTTAAGAAAAAAGAAGAGGA
TTAATCTTGGTTTTCCCCTTGGT.

17. CVL80

The nucleic acid sequence for CVL80 is:

(SEQ ID NO: 76)
ACCAGGGGGAAAACGAAGTGGTGACTCAAATCATCGAAGACCCTCGAGA
TTACATAGGTCCGGAACCTATGGCCTTCGTGACCGACCTCGAGTCAGAG
TAGTTCAATAAGGACCTATCAAGTTTGGGCAATTTTTCGTCCCCGACAC
AAAAATGTCATCCGTGCTTAAAGCATATGAGCGATTCACACTCACTCAA
GAACTGCAAGATCAGAGTGAGGAAGGTACAATCCCACCTACAACACTAA
AACCGGTAATCAGGGTATTTATACTAACCTCTAATAACCCAGAGCTAAG
ATCCCGGCTTCTTCTATTCTGCCTACGGATTGTTCTCAGTAATGGTGCA
AGGGATTCCCATCGCTTTGGAGCATTACTTACAATGTTTTCGCTACCAT
CAGCCACAATGCTCAATCATGTCAAATTAGCTGACCAGTCACCAGAAGC
TGATATCGAAAGGGTAGAGATCGATGGCTTTGAGGAGGGATCATTCCGC
TTAATCCCCAATGCTCGTTCAGGTATGAGCCGTGGAGAGATCAATGCCT
ATGCTGCACTTGCAGAAGATCTACCTGACACACTAAACCATGCAACACC
TTTTGTTGATTCCGAAGTCGAGGGAACTGCATGGGATGAGATTGAGACT
TTCTTAGATATGTGTTACAGTGTCCTAATGCAGGCATGGATAGTGACTT
GCAAGTGCATGACTGCGCCAGACCAACCTGCTGCTTCTATTGAGAAACG
CCTGCAAAAATATCGTCAGCAAGGCAGGATCAACCCGAGATATCTCCTG
CAACCGGAGGCTCGAAGAATAATCCAGAATGTAATCCGAAAGGGAATGG
TGGTCAGACATTTCCTCACCTTTGAACTGCAGCTTGCCCGAGCACAAAG
CCTTGTATCAAATAGGTATTATGCTATGGTAGGGGATGTTGGAAAGTAT
ATAGAGAATTGTGGAATGGGAGGCTTCTTTTTGACACTAAAATATGCAT
TAGGAACCAGATGGCCCACACTTGCTTTAGCTGCATTTTCAGGAGAGCT
AACAAAGCTAAAGTCCCTCATGGCATTATACCAGACCCTTGGTGAGCAG
GCCCGATATTTGGCCCTATTGGAGTCACCACATTTGATGGATTTTGCTG
CAGCAAACTACCCACTGCTATATAGCTATGCTATGGGAATAGGCTATGT
GTTAGATGTCAACATGAGGAACTACGCTTTCTCCAGATCATACATGAAT
AAGACATATTTCCAATTGGGAATGGAAACTGCAAGAAAACAACAGGGTG
CAGTTGACATGAGGATGGCAGAAGATCTCGGTCTAACTCAAGCCGAACG
CACCGAGATGGCAAATACACTTGCCAAATTGACCACAGCAAATCGAGGG
GCAGACACCAGGGGAGGAGTCAACCCGTTCTCATCTATCACTGGGACAA
CTCAGGTGCCCGCTGCAGCAACAGGTGACACATTCGAGAGTTACATGGC
AGCGGATCGACTGAGGCAGAGATATGCTGATGCAGGCACCCACGATGAT
GAGATGCCACCATTGGAAGAGGAGGAAGAGGACGACACATCTGCAGGTC
CACGCACTGGACTAACTCTTGAACAAGTGGCCTTGGACATCCAGAACGC
AGCAGTTGGAGCTCCCATCCATACAGATGACCTGAATGCCGCACTGGGT
GATCTTGACATCTAGACAATTCAGATCCCAATCTTAAATCGACACACCT
AATTGACCAGTTAGATGGAACTACAGTGGATTCCATAAGGTTCCTGCCT
ACCATCGGCTTTTAAGAAAAAAATAGGCCCGGACGGGTTAGCAACAAGC
GACTGCCGATGCCAATAACACAATCCACAATCTACAATGGATCCCACTG
ATCTGAGCTTCTCCCCAGATGAGATCAATAAGCTCATAGAGACAGGCCT
GAATACTGTGGAGTATTTTACTTCCCAACAAGTCACAGGAACATCCTCT
CTTGGAAAGAATACAATACCACCAGGGGTCACAGGACTACTAACCAATG
CTGCAGAGGCAAAGATCCAAGAGTCAACCAACCATCAGAAGGGTTCAGT
TGGTGGGGGCGCAAAACCAAAGAAACCGCGACCAAAAATTGCCATTGTG
CCAGCAGATGACAAAACGGTGCCCGGAAAGCCGATCCCAAACCCTCTAT
TAGGTCTGGACTCCACCCCGAGCACCCAAACTGTGCTTGATCTAAGTGG
GAAAACATTACCATCAGGATCCTATAAGGGGGTTAAACTTGCGAAATTT
GGAAAAGAAAATCTGATGACACGGTTCATCGAGGAACCCAGAGAGAATC
CTATCGCAACCAATTCCCCCATCGATTTTAAGAGGGGCAGGGATACCGG
CGGGTTCCATAGAAGGGAGTACTCAATCGGATGGGTGGGAGATGAAGTC
AAGGTCACTGAGTGGTGCAATCCATCCTGTTCTCCAATCACCGCTGCAG
CAAGGCGATTTGAATGCACTTGTCACCAGTGTCCAGTCACTTGCTCTGA
ATGTGAACGAGATACTTAATACAGTGAGAAATTTGGACTCTCGGATGAA
TCAACTGGAGACAAAAGTAGATCGCATTCTCTCATCTCAGTCTCTAATC
CAGACCATCAAGAATGACATAGTTGGACTTAAAGCAGGGATGGCTACTT
TAGAAGGAATGATTACAACTGTGAAAATCATGGACCCGGGAGTTCCCAG
TAATGTTACTGTGGAAGATGTACGCAAGAAACTAAGTAACCATGCTGTT
GTTGTGCCAGAATCATTCAATGATAGTTTCTTGACTCAATCTGAAGATG
TAATTTCACTTGATGAGTTGGCTCGACCAACTGCAACAAGTGTTAAGAA
GATTGTCAGGAAGGTTCCTCCTCAGAAGGATCTGACTGGATTGAAGATC
ACACTAGAGCAATTGGCAAAGGATTGCATCAGCAAACCGAAGATGAGGG
AAGAGTATCTCCTCAAAATCAACCAGGCTTCCAGTGAGGCTCAGCTAAT
TGACCTCAAGAAAGCAATCATCCGCAGTGCAATTTGATCAAGAAACACC
CAATTACACTACACTGGTATGACACTGTACTAACCCTGAGGGTTTTAGA
AAAAACGATTAACGATAAATAAGCCCGAACACTACACACCACCCGAGGC
AGCCATGCCATCCATCAGCATCCCCGCAGACCCCACCAATCCACGTCAA
TCAATAAAAGCGTTCCCAATTGTGATCAACCGTGATGGGGGTGAGAAAG
GTCGCTTGGTTAAACAACTACGCACAACCTACTTGAATGACCTAGATAC
TCATGAGCCACTGGTGACATTCGTAAATACTTATGGATTCATCTACGAA
CAGGATCGGGGAAATACCATTGTCGGAGAGGATCAACATGGGAAGAAAA
GAGAGGCTGTGACTGCTGCAATGGTTACCCTTGGATGTGGGCCTAATCT
ACCATCATTAGGGAATGTCCTGGGACAACTGAGTGAATTCCAGGTCATT
GTTAGGAAGACATCCAGCAAAGCGGAAGAGATGGTCTTTGAAATTGTTA
AGTATCCGAGAATATTTCGGGGTCATACATTAATCCAGAAAGGACTAGT
CTGTGTCTCCGCAGAAAAATTTGTTAAGTCACCAGGGAAAGTACAATCT
GGAATGGACTATCTCTTCATTCCGACATTTCTGTCAGTGACTTACTGTC
CAGCTGCAATCAAATTTCAGGTACCTGGCCCCATGTTGAAAATGAGATC
AAGATACACTCAGAGCTTACAACTTGAACTAATGATAAGAATCCTGTGT
AAGCCCGATTCGCCACTTATGAAGGTCCATATCCCTGACAAGGAAGGAA
GAGGATGTCTTGTATCAGTATGGTTGCATGTATGCAATATCTTCAAATC
AGGAAACAAGAATGGCAGTGAGTGGCAGGAATACTGGATGAGAAAGTGT
GCTAACATGCAACTTGAAGTGTCGATTGCAGATATGTGGGGACCAACTA
TCATAATTCATGCCAGAGGTCACATTCCCAAAAGTGCTAAGTTGTTTTT
TGGAAAGGGTGGATGGAGCTGCCATCCACTTCACGAAGTTGTTCCAAGT
GTCACTAAAACACTATGGTCCGTGGGCTGTGAGATTACAAAGGCGAAGG
CAATAATACAAGAGAGTAGCATCTCTCTTCTCGTGGAGACTACTGACAT
CATAAGTCCAAAAGTCAAAATTTCATCTAAGCATCGCCGCTTTGGGAAA
TCAAATTGGGGTCTGTTCAAGAAAACCAAATCACTGCCCAACCTGACGG
AGCTGGAATGACTGACCCCCAATCGAGACTACACCACCTCAAACTATAG
GTGGGTGGTACCTCAGTGATTAATCTCGTAAGCACTGATCGTAGGCTAC
AACACACTAATATTATCCAGATTAGAGAGCTTAATTAGCTCTGTATTAA
TAATAACACTACTATTCCAATAACTGGAATCACCAGCTTGATTTATCTC
CAAAATGATTCAAAGAAAACAAATCATATTAAGACTATCCTAAGCACGA
ACCCATATCGTCCTTCAAATCATGGGTACTATAATTCAATTTCTGGTGG
TCTCCTGTCTATTGGCAGGAGCAGGCAGTCTTGATCCAGCAGCCCTCAT
GCAAATCGGTGTCATTCCAACAAATGTCCGGCAACTTATGTATTATACT
GAGGCCTCATCAGCATTCATTGTTGTGAAGTTAATGCCTACAATTGACT
CGCCGATTAGTGGATGTAATATAACATCAATTTCAAGCTATAATGCAAC
AGTGACAAAACTCCTACAGCCGATCGGTGAGAATTTGGAGACGATTAGG
AACCAGTTGATTCCAACTCGGAGGAGACGCCGGTTTGCAGGGGTGGTGA
TTGGATTAGCTGCATTAGGAGTAGCTACTGCCGCACAGGTCACTGCCGC
AGTAGCACTAGTAAAGGCAAATGAAAATGCTGCGGCTATACTCAATCTC
AAAAATGCAATCCAAAAAACAAATGCAGCAGTTGCAGATGTGGTCCAGG
CCACACAATCACTAGGAACaGCAGTTCAAGCAGTTCAAGATCACATAAA
CAGTGTGGTAAGTCCAGCAATTACAGCAGCCAATTGTAAAGCCCAAGAT
GCTATCATTGGCTCAATCCTCAATCTCTATTTGACCGAGTTGACAACTA
TCTTCCACAATCAAATTACAAACCCTGCATTGAGTCCTATTACAATTCA
AGCTTTAAGGATCCTACTAGGGAGTACCTTGCCGACTGTGGTCGAAAAA
TCTTTCAATACCCAGATAAGTGCGGCTGAGCTTCTCTCATCAGGGTTAT
TGACAGGCCAGATTGTGGGATTAGATTTGACCTATATGCAGATGGTCAT
AAAAATTGAGCTGCCAACTTTAACTGTACAACCTGCAACCCAGATCATA
GATCTGGCCACCATTTCTGCATTCATTAACAATCAAGAAGTCATGGCCC
AATTACCAACACGTGTTATTGTGACTGGCAGCTTGATCCAAGCCTATCC
CGCATCGCAATGCACTATTACACCCAACACTGTGTACTGTAGGTATAAT
GATGCCCAAGTACTCTCAGATGATACGATGGCTTGCCTCCAAGGTAACT
TGACAAGATGCACCTTCTCTCCAGTGGTTGGGAGCTTTCTCACTCGATT
CGTGCTGTTCGATGGAATAGTTTATGCAAATTGCAGGTCGATGTTGTGC
AAGTGCATGCAGCCTGCTGCTGTGATCCTACAGCCGAGTTCATCCCCTG
TAACTGTCATTGACATGTACAAATGTGTGAGTCTGCAGCTTGACAATCT
CAGATTCACCATCACTCAATTGGCCAATGTAACCTACAATAGCACCATC
AAGCTTGAAACATCCCAGATCTTGCCTATTGATCCGTTGGATATATCCC
AGAATCTAGCTGCGGTGAATAAGAGTCTAAGTGATGCACTACAACACTT
AGCACAAAGTGACACATACCTTTCTGCAATCACATCAGCTACGACTACA
AGTGTATTATCCATAATAGCAATCTGTCTTGGATCGTTAGGTTTAATAT
TAATAATCTTGCTCAGTGTAGTTGTGTGGAAGTTATTGACCATTGTCGC
TGCTAATCGAAATAGAATGGAGAATTTTGTTTATCATAATTCAGCATTC
CACCACTCACGATCTGATCTCAGTGAGAAAAATCAACCTGCAACTCTTG
GAACAAGATAAGACAGTCATCCATTAGTAATTTTTAAGAAAAAAACGAT
AGGACCGAAACTAGTATTGAAAGAACCGTCTCGGTCAATCTAGGTAATC
GAGCTGcTACCGTCTCGGAAAGCTCAAATCATGCTGCCTGATCCGGAAG
ATCCGGAAAGCAAAAAAGCTACAAGGAGAACAGGAAACCTAATTATCTG
CTTCCTATTCATCTTCTTTCCGTTTGTAAACTTCATTGTTCCAACTCTA
AGACACTTGCTGTCCTAACACCTGCTATAGGCTATCCACTGCATCATCT
CTTTTTAAGAAAAAAATAGGCCCGGACGGGTTAGCAACAAGCGACTGCC
GGTGCCAACAACACAATCCACAATCTACAGTCGACGCGGCCGCCACCat
gttcgtcttcctggtcctgctgcctctggtctcctcacagtgcgtcaat
ctgacaactcggactcagctgccacctgcttatactaatagcttcacca
gaggcgtgtactatcctgacaaggtgtttagaagctccgtgctgcactc
tacacaggatctgtttctgccattctttagcaacgtgacctggttccac
GTGatcagcggcaccaatggcacaaagcggttcgacaatcccgtgctgc
cttttaacgatggcgtgtacttcgcctctATCgagaagagcaacatcat
cagaggctggatctttggcaccacactggactccaagacacagtctctg
ctgatcgtgaacaatgccaccaacgtggtcatcaaggtgtgcgagttcc
agttttgtaatgatcccttcctgGATcacaagaacaataagagctggat
ggagtccgagtttagagtgtattctagcgccaacaactgcacatttgag
tacgtgagccagcctttcctgatggacctggagggcaagcagggcaatt
tcaagaacctgagggagttcgtgtttaagaatatcgacggctacttcaa
aatctactctaagcacacccccATCATCgtgcgcGAGCCTGAGgacctg
cctcagggcttcagcgccctggagcccctggtggatctgcctatcggca
tcaacatcacccggtttcagacactgctggccctgcacagaagctacct
gacacccggcgactcctctagcggatggaccgccggcgctgccgcctac
tatgtgggctacctccagccccggaccttcctgctgaagtacaacgaga
atggcaccatcacagacgcagtggattgcgccctggaccccctgagcga
gacaaagtgtacactgaagtcctttaccgtggagaagggcatctatcag
acatccaatttcagggtgcagccaaccgagtctatcgtgcgctttccta
atatcacaaacctgtgcccatttGACgaggtgttcaacgcaacccgctt
cgccagcgtgtacgcctggaataggaagcggatcagcaactgcgtggcc
gactatagcgtgctgtacaacCTGgccCCCttcTTCacctttaagtgct
atggcgtgtcccccacaaagctgaatgacctgtgctttaccaacgtcta
cgccgattctttcgtgatcaggggcgacgaggtgcgccagatcgccccc
ggccagacaggcAATatcgcagactacaattataagctgccagacgatt
tcaccggctgcgtgatcgcctggaacagcaacAAGctggattccaaagt
gTCCggcaactacaattatctgtaccggctgtttagaaagagcaatctg
aagcccttcgagagggacatctctacagaaatctaccaggccggcAATA
AGccttgcaatggcgtgGCCggctttaactgttatttcccactcAAGtc
ctacTCCttcCGCcccacaTATggcgtgggcCACcagccttaccgcgtg
gtggtgctgagctttgagctgctgcacgccccagcaacagtgtgcggcc
ccaagaagtccaccaatctggtgaagaacaagtgcgtgaacttcaactt
caacggcctgAAGggcacaggcgtgctgaccgagtccaacaagaagttc
ctgccatttcagcagttcggcagggacatcgcagataccacagacgccg
tgcgcgacccacagaccctggagatcctggacatcacaccctgctcttt
cggcggcgtgagcgtgatcacacccggcaccaatacaagcaaccaggtg
gccgtgctgtatcagGGCgtgaattgtaccgaggtgcccgtggctatcc
acgccgatcagctgaccccaacatggcgggtgtacagcaccggctccaa
cgtcttccagacaagagccggatgcctgatcggagcagagTACgtgaac
aattcctatgagtgcgacatcccaatcggcgccggcatctgtgcctctt
accagacccagacaAAGtctCACagaagagcccggagcgtggcctccca
gtctatcatcgcctataccatgtccctgggcgccgagaacagcgtggcc
tactctaacaatagcatcgccatcccaaccaacttcacaatctctgtga
ccacagagatcctgcccgtgtccatgaccaagacatctgtggactgcac
aatgtatatctgtggcgattctaccgagtgcagcaacctgctgctccag
tacggcagcttttgtacccagctgAAGagagccctgacaggcatcgccg
tggagcaggataagaacacacaggaggtgttcgcccaggtgaagcaaat
ctacaagaccccccctatcaagTACtttggcggcttcaatttttcccag
atcctgcctgatccatccaagccttctaagcggagctttatcgaggacc
tgctgttcaacaaggtgaccctggccgatgccggcttcatcaagcagta
tggcgattgcctgggcgacatcgcagccagggacctgatctgcgcccag
aagtttAAGggcctgaccgtgctgccacccctgctgacagatgagatga
tcgcacagtacacaagcgccctgctggccggcaccatcacatccggatg
gaccttcggcgcaggagccgccctccagatcccctttgccatgcagatg
gcctataggttcaacggcatcggcgtgacccagaatgtgctgtacgaga
accagaagctgatcgccaatcagtttaactccgccatcggcaagatcca
ggacagcctgtcctctacagccagcgccctgggcaagctccaggatgtg
gtgaatCACaacgcccaggccctgaataccctggtgaagcagctgagca
gcAAGttcggcgccatctctagcgtgctgaatgacatcTTTagccggct
ggacaaggtggaggcagaggtgcagatcgaccggctgatcaccggccgg
ctccagagcctccagacctatgtgacacagcagctgatcagggccgccg
agatcagggccagcgccaatctggcagcaaccaagatgtccgagtgcgt
gctgggccagtctaagagagtggacttttgtggcaagggctatcacctg
atgtccttccctcagtctgccccacacggcgtggtgtttctgcacgtga
cctacgtgcccgcccaggagaagaacttcaccacagcccctgccatctg
ccacgatggcaaggcccactttccaagggagggcgtgttcgtgtccaac
ggcacccactggtttgtgacacagcgcaatttctacgagccccagatca
tcaccacagacaacaccttcgtgagcggcaactgtgacgtggtcatcgg
catcgtgaacaataccgtgtatgatccactccagcccgagctggacagc
tttaaggaggagctggataagtatttcaagaatcacacctcccctgacg
tggatctgggcgacatcagcggcatcaatgcctccgtggtgaacatcca
gaaggagatcgaccgcctgaacgaggtggctaagaatctgaacgagagc
ctgatcgacctccaggagctgggcaagtatgagcagtacatcaagtggc
cctggtacatctggctgggcttcatcgccggcctgatcgccatcgtgat
ggtgaccatcatgctgtgctgtatgacatcctgctgttcttgcctgaag
ggctgctgtagctgtggctcctgctgtAAGTTATTGACCATTGTCGTTG
CTAATCGAAATAGAATGGAGAATTTTGTTTATCATAAATGAGCTAGCCT
CCTGCCATACTTCCTACTCACATCATATCTATTTTAAAGAAAAAATAGG
CCCGAACACTAATCGTGCCGGCAGTGCCACTGCACACACAACACTACAC
ATACAATACACTACAATGGTTGCAGAAGATGCCCCTGTTAGGGGCACTT
GCCGAGTATTATTTCGAACAACAACTTTAATTTTTCTATGCACACTATT
AGCATTAAGCATCTCTATCCTTTATGAGAGTTTAATAACCCAAAAGCAA
ATCATGAGCCAAGCAGGCTCAACTGGATCTAATTCTAGATTAGGAAGTA
TCACTGATCTTCTTAATAATATTCTCTCTGTCGCAAATCAGATTATATA
TAACTCTGCAGTCGCTCTACCTCTACAATTGGACACTCTTGAATCAACA
CTCCTTACAGCCATTAAGTCTCTTCAAACAAGTGACAAGCTAGAACAGA
ACTGCTCGTGGGGTGCTGCACTGATTAATGATAATAGATACATTAATGG
CATCAATCAGTTCTATTTCTCAATTGCTGAGGGTCGCAAGCTGACACTT
GGCCCACTTCTTAATATACCTAGTTTCATTCCAACTGCCACGACACCAG
AGGGCTGCACCAGGATCCCATCATTCTCGCTCACCAAGACACACTGGTG
TTATACACACAATGTTATCCTGAATGGATGCCAGGATCATGTATCCTCA
AATCAATTTGTTTCCATGGGAATCATTGAACCCACTTCTGCCGGGTTTC
CATCCTTTCGAACCCTAAAGACTCTATATCTCAGCGATGGGGTCAATCG
TAAGAGCTGCTCTATCAGTACAGTTCCGGGGGGTTGTATGATGTACTGT
TTCGTCTCTACTCAACCAGAGAGGGATGACTACTTTTCTACCGCTCCTC
CAGAACAACGAATTATTATAATGTACTATAATGATACAATCGTGGAGCG
CATAATTAATCCACCCGGGGTACTAGATGTATGGGCAACATTGAACCCA
GGAACAGGAAGCGGGGTATATTATTTAGGTTGGGTACTCTTTCCAATAT
ATGGCGGCGTGATTAAAGATACGAGTTTATGGAATAATCAAGCAAATAA
ATACTTTATCCCCCAGATGGTTGCTGCTCTCTGCTCACAAAACCAGGCA
ACTCAAGTCCAAAATGCTAAGTCATCATACTATAGCAGCTGGTTTGGCA
ATCGAATGATTCAGTCTGGGATCCTGGCATGTCCTCTTCAACAGGATCT
AACCAATGAGTGTTTAGTTCTGCCCTTTTCTAATGATCAGGTGCTTATG
GGTGCTGAAGGGAGATTATACATGTATGGTGACTCGGTGTATTACTACC
AAAGAAGCAATAGTTGGTGGCCTATGACCATGCTGTATAAGGTAACCAT
AACATTCACTAATGGTCAGCCATCTGCTATATCAGCTCAGAATGTGCCC
ACACAGCAGGTCCCTAGACCTGGGACAGGAAGCTGCTCTGCAACAAATA
GATGTCCCGGTTTTTGCTTGAAAGGAGTGTATGCTGATGCCTGGTTACT
GACCAACCCTTCGTCTACCAGTACATTTGGATCAGAAGCAACCTTCACT
GGTTCTTATCTCAACGCAGCAACTCAGCGTATCAATCCGACGATGTATA
TCGCGAACAACACACAGATCATAAGCTCACAGCAATTTGGATCAAGCGG
TCAAGAAGCAGCATATGGCCACACAACTTGTTTTAGGGACACAGGCTCT
GTTATGGTATACTGTATCTATATTATTGAATTGTCCTCATCTCTCTTAG
GACAATTTCAGATTGTCCCATTTATCCGTCAGGTGACACTATCCTAAAG
GCAGAAGCCTCCAGGTCTGACCCAGCCAATCAAAGCATTATACCAGACC
ATGGAATGCATACCAAACATTATTGACACTAATGACACACAAAATTGGT
TTTAAGAAAAACCAAGAGAACAATAGGCCAGAATGGCTGGGTCTCGGGA
GATATTACTCCCTGAAGTCCATCTCAATTCACCAATTGTAAAGCATAAG
CTATACTATTACATTCTACTTGGAAACCTCCCAAATGAGATCGACATTG
ACGATTTAGGTCCATTACATAATCAAAATTGGAATCAAATAGCACATGA
AGAGTCTAACTTAGCCCAACGCTTGGTAAATGTAAGAAATTTTCTAATT
ACCCACATCCCTGATCTTAGAAAGGGCCATTGGCAAGAGTATGTCAATG
TAATACTGTGGCCGCGAATTCTTCCCTTGATCCCGGATTTTAAAATCAA
TGACCAATTGCCTCTACTCAAAAATTGGGACAAGTTAGTTAAAGAATCA
TGTTCAGTAATCAATGCGGGTACTTCCCAGTGCATTCAGAATCTCAGCT
ATGGACTGACAGGTCGTGGGAACCTCTTTACACGATCACGTGAACTCTC
TGGTGACCGCAGGGATATTGATCTTAAGACGGTTGTGGCAGCATGGCAT
GACTCAGACTGGAAAAGAATAAGTGATTTTTGGATTATGATCAAATTCC
AGATGAGACAATTAATTGTTAGGCAAACAGATCATAATGATCCTGATTT
AATCACGTATATCGAAAATAGAGAAGGCATAATCATCATAACCCCTGAA
CTGGTAGCATTATTTAACACTGAGAATCATACACTAACATACATGACCT
TTGAAATTGTACTGATGGTTTCAGATATGTACGAAGGTCGTCACAACAT
TTTATCACTATGCACAGTTAGCACTTACCTGAATCCTCTGAAGAAAAGA
ATAACATATTTATTGAGCCTTGTAGATAACTTAGCTTTTCAGATAGGTG
ATGCTGTATATAACATAATTGCTTTGCTAGAATCCTTTGTATATGCACA
GTTGCAAATGTCAGATCCCATCCCAGAACTCAGAGGACAATTCCATGCA
TTCGTATGTTCTGAGATTCTTGATGCACTAAGGGGAACTAATAGTTTCA
CCCAGGATGAATCAAGAACTGTGACAACCAATTTGATATCCCCATTCCA
AGATCTGACCCCAGATCTTACGGCTGAATTGCTCTGTATAATGAGGCTT
TGGGGACACCCCATGCTCACCGCCAGTCAAGCTGCGGGAAAGGTACGCG
AGTCCATGTGTGCTGGAAAAGTATTAGACTTTCCCACCATTATGAAAAC
ACTAGCCTTTTTCCATACTATTCTGATCAATGGATACAGGAGGAAGCAT
CATGGAGTATGGCCACCCTTAAACTTACCGGGTAATGCTTCAAAGGGTC
TCACGGAACTTATGAATGACAATACTGAGATAAGCTATGAATTCACACT
TAAGCATTGGAAGGAAATCTCTCTTATAAAATTCAAGAAATGTTTTGAT
GCAGACGCAGGTGAGGAACTCAGTATATTTATGAAAGATAAAGCAATTA
GTGCCCCAAAACAAGATTGGATGAGTGTGTTTAGAAGAAGCCTAATCAA
ACAGCGCCATCAGCATCATCAGGTCCCCCTACCAAATCCATTCAATCGA
CGGCTATTGCTAAACTTTCTCGGAGATGACAAATTCGACCCGAATGTGG
AGCTACAGTATGTAACATCAGGTGAGTATCTACATGATGACACGTTTTG
TGCATCATATTCACTAAAAGAGAAGGAAATTAAACCTGATGGTCGAATT
TTTGCAAAGTTGACTAAGAGAATGAGATCATGTCAAGTTATAGCAGAAT
CTCTTTTAGCGAACCATGCTGGGAAGTTAATGAAAGAGAATGGTGTTGT
GATGAATCAGCTATCATTAACAAAATCACTATTAACAATGAGTCAGATT
GGAATAATATCCGAGAAAGCTAGAAAATCGACTCGAGATAACATAAATC
AACCTGGTTTCCAGAATATCCAGAGAAATAAATCACATCACTCCAAGCA
AGTCAATCAGCGAGATCCAAGTGATGACTTTGAATTGGCAGCATCTTTT
TTAACTACTGATCTCAAAAAATATTGTTTACAATGGAGGTACCAGACAA
TTATCCCATTTGCTCAATCATTAAACAGAATGTATGGTTATCCTCATCT
CTTTGAGTGGATTCACTTACGGCTAATGCGTAGTACACTTTACGTGGGG
GATCCCTTCAACCCACCAGCAGATACCAGTCAATTTGATCTAGATAAAG
TAATTAATGGAGATATCTTCATTGTATCACCCAGAGGTGGAATTGAAGG
GCTATGTCAAAAGGCTTGGACAATGATATCTATCTCTGTGATAATTCTA
TCTGCCACAGAGTCTGGCACACGAGTAATGAGTATGGTGCAGGGAGATA
ATCAAGCAATTGCTGTCACCACACGAGTACCAAGGAGCCTGCCGACTCT
TGAGAAAAAGACTATTGCTTTTAGATCTTGTAATCTATTCTTTGAGAGG
TTAAAATGTAATAATTTTGGATTAGGTCACCATTTGAAAGAACAAGAGA
CTATCATTAGTTCTCACTTCTTTGTTTATAGCAAGAGAATATTCTATCA
GGGGAGGATTCTAACGCAAGCCTTAAAAAATGCTAGTAAGCTCTGCTTG
ACAGCTGATGTCCTAGGAGAATGTACCCAATCATCATGTTCTAATCTTG
CAACTACTGTCATGAGGTTAACTGAGAATGGTGTTGAAAAAGATATCTG
TTTCTACTTGAATATCTATATGACCATCAAACAGCTCTCCTATGATATC
ATCTTCCCTCAAGTGTCAATTCCTGGAGATCAGATCACATTAGAATACA
TAAATAATCCACACCTGGTATCACGATTGGCTCTTCTGCCATCCCAGCT
AGGAGGTCTAAACTACCTGTCATGCAGTAGGCTGTTCAATCGAAACATA
GGCGACCCGGTGGTTTCCGCAGTTGCAGATCTTAAGAGATTAATTAAAT
CAGGATGTATGGATTACTGGATCCTTTATAACTTATTAGGGAGAAAACC
GGGAAACGGCTCATGGGCTACTTTAGCAGCTGACCCGTACTCAATCAAT
ATAGAGTATCAATACCCCCCAACTACAGCTCTTAAGAGGCACACCCAAC
AAGCTCTGATGGAACTCAGTACGAATCCAATGTTACGTGGCATATTCTC
TGACAATGCACAGGCAGAAGAAAATAATCTTGCTAGATTTCTCCTGGAT
AGGGAGGTGATCTTTCCGCGTGTAGCTCACATCATCATTGAGCAAACCA
GTGTCGGGAGGAGAAAACAGATTCAAGGATATTTGGATTCAACTAGATC
GATAATGAGTAAATCACTAGAAATTAAGCCCTTGTCCAATAGGAAGCTT
AATGAAATACTGGATTACAACATCAATTACCTAGCTTACAATTTGGCAT
TACTCAAGAATGCTATTGAACCTCCGACTTATTTGAAAGCAATGACTCT
TGAAACATGTAGCATCGACATTGCAAGGAGCCTCCGGAAGCTCTCCTGG
GCCCCACTCTTGGGTGGGAGAAATCTTGAAGGATTAGAGACGCCAGATC
CCATTGAAATTACTGCAGGAGCATTAATTGTTGGATCGGGCTACTGTGA
ACAGTGTGCTGCAGGAGACAATCGATTCACATGGTTTTTCTTGCCATCT
GGTATCGAGATAGGAGGGGATCCCCGTGATAATCCTCCTATCCGTGTAC
CGTACATTGGCTCCAGGACTGATGAGAGGAGGGTAGCCTCAATGGCATA
CATCAGGGGTGCCTCGAGTAGCCTAAAAGCAGTTCTTAGACTGGCGGGA
GTGTACATCTGGGCATTCGGAGATACTCTGGAGAATTGGATAGATGCAC
TGGATTTGTCTCACACTAGAGTTAACATCACACTTGAACAGCTGCAATC
CCTCACCCCACTTCCAACCTCTGCCAATCTAACCCATCGGTTGGATGAT
GGCACAACTACCCTAAAGTTTACTCCTGCGAGCTCTTACACCTTTTCAA
GTTTCACTCATATATCAAATGATGAGCAATACCTGACAATTAATGACAA
AACTGCAGATTCAAATATAATCTACCAACAGTTAATGATCACTGGACTC
GGAATTTTAGAAACATGGAATAATCCCCCAATCAATAGAACATTCGAAG
AATCTACCCTACATTTGCACACTGGTGCATCATGTTGTGTCCGACCTGT
GGACTCCTGCATCATCTCAGAAGCATTAACAGTCAAGCCACATATTACA
GTACCGTACAGCAATAAATTTGTATTTGATGAAGACCCGCTATCTGAAT
ATGAGACTGCAAAACTGGAATCGTTATCATTTCAAGCCCAATTAGGCAA
CATTGATGCTGTAGATATGACAGGTAAATTAACATTATTGTCCCAATTC
ACTGCAAGGCAGATTATTAATGCAATCACTGGACTCGATGAGTCTGTCT
CTCTTACTAATGATGCCATTGTTGCATCAGACTATGTCTCCAATTGGAT
TAGTGAATGCATGTATACCAAATTAGATGAATTATTTATGTATTGTGGG
TGGGAACTACTATTGGAACTATCCTATCAAATGTATTATCTGAGGGTAG
TTGGGTGGAGTAATATAGTGGATTATTCTTACATGATCTTGAGAAGAAT
CCCGGGTGCAGCATTAAACAATCTGGCATCTACATTAAGTCATCCAAAA
CTTTTCCGACGAGCTATCAACCTAGATATAGTTGCCCCCTTAAATGCTC
CTCATTTTGCATCTCTGGACTACATCAAGATGAGTGTGGATGCAATACT
CTGGGGCTGTAAAAGAGTCATCAATGTGCTCTCCAATGGAGGGGACTTA
GAATTAGTTGTGACATCTGAAGATAGCCTTATTCTCAGTGACCGATCCA
TGAATCTCATTGCAAGGAAATTAACTTTATTATCACTGATTCACCATAA
TGGTTTGGAACTACCAAAGATTAAGGGGTTCTCTCCTGATGAGAAGTGT
TTCGCTTTGACAGAATTTTTGAGGAAAGTGGTGAACTCAGGGTTGAGTT
CAATAGAGAACCTATCAAATTTTATGTACAATGTGGAGAACCCACGGCT
TGCAGCATTCGCCAGCAACAATTACTACCTGACCAGAAAATTATTGAAT
TCAATACGAGATACTGAGTCGGGTCAAGTAGCAGTCACCTCATATTATG
AATCATTAGAATATATTGATAGTCTTAAGCTAACCCCACATGTGCCTGG
TACCTCATGCATTGAGGATGATAGTCTATGTACAAATGATTACATAATC
TGGATCATAGAGTCTAATGCAAACTTGGAGAAGTATCCAATTCCAAATA
GCCCTGAGGATGATTCCAATTTCCATAACTTTAAGTTGAATGCTCCATC
GCACCATACCTTACGCCCATTAGGGTTGTCATCAACTGCTTGGTATAAG
GGTATAAGCTGTTGCAGGTACCTTGAGCGATTAAAGCTACCACAAGGTG
ATCATTTATATATTGCAGAAGGTAGTGGTGCCAGTATGACAATCATAGA
ATACCTATTCCCAGGAAGAAAGATATATTACAATTCTTTATTTAGTAGT
GGTGACAATCCCCCACAAAGAAATTATGCACCAATGCCTACTCAGTTCA
TTGAGAGTGTCCCATACAAGCTCTGGCAAGCACACACAGATCAATATCC
CGAGATTTTTGAGGACTTCATCCCTCTATGGAACGGAAATGCCGCCATG
ACTGACATAGGAATGACAGCTTGTGTAGAATTCATCATCAATCGAGTCG
GCCCAAGGACTTGCAGTTTAGTACATGTAGATTTGGAATCAAGTGCAAG
CTTAAATCAACAATGCCTGTCAAAGCCGATAATTAATGCTATCATCACT
GCTACAACTGTTTTGTGCCCTCATGGGGTGCTTATTCTGAAATATAGTT
GGTTGCCATTTACTAGATTTAGTACTTTGATCACTTTCTTATGGTGCTA
CTTTGAGAGAATCACTGTTCTTAGGAGCACATATTCTGGTCCAGCTAAT
CATGAGGTTTATTTAATTTGTATCCTTGCCAACAACTTTGCATTCCAGA
CTGTCTCGCAGGCAACAGGAATGGCGATGACTTTAACCGATCAAGGGTT
TACTTTGATATCACCTGAAAGAATAAATCAGTATTGGGATGGTCACTTG
AAGCAAGAACGTATCGTAGCAGAAGCAATTGATAAGGTGGTTCTAGGAG
AAGATGCTCTATTCAATTCGAGTGATAATGAATTAATTCTCAAATGTGG
AGGGACACCAAATGCACGGAATCTTATCGATATCGAGCCAGTCGCAACT
TTCATAGAATTTGAACAACTGATCTGCACAATGTTGACAACCCACTTGA
AGGAAATAATTGATATAACAAGGTCTGGAACCCAGGATTATGAAAGTTT
ATTACTCACTCCTTACAATTTAGGTCTTCTTGGTAAAATCAGTACGATA
GTGAGATTATTAACAGAAAGGATTCTAAATCATACTATCAGGAATTGGT
TGATCCTCCCACCTTCGCTCCGGATGATCGTGAAGCAGGACTTGGAATT
CGGCATATTCAGGATTACTTCCATCCTCAATTCTGATCGGTTCCTGAAG
CTTTCTCCAAATAGGAAATACTTGATTACACAATTAACTGCAGGCTACA
TTAGGAAATTGATTGAGGGGGATTGTAATATCGATCTAACCAGACCTAT
CCAAAAACAAATCTGGAAAGCATTAGGTTGTGTAGTCTATTGTCACGAT
CCAGTAGATCAAAGGGAATCAACAGAGTTTATTGATATAAATATTAATG
AAGAAATAGACCGCGGGATCGATGGCGAGGAAATCTAAATATATCAAGA
ATCAGAATTAGTTTAAGAAAAAAGAAGAGGATTAATCTTGGTTTTCCCC
TTGGT.

18. CVL86

The nucleic acid sequence for CVL86 is:

(SEQ ID NO: 77)
ACCAGGGGGAAAACGAAGTGGTGACTCAAATCATCGAAGACCCTCGAGA
TTACATAGGTCCGGAACCTATGGCCTTCGTGACCGACCTCGAGTCAGAG
TAGTTCAATAAGGACCTATCAAGTTTGGGCAATTTTTCGTCCCCGACAC
AAAAATGTCATCCGTGCTTAAAGCATATGAGCGATTCACACTCACTCAA
GAACTGCAAGATCAGAGTGAGGAAGGTACAATCCCACCTACAACACTAA
AACCGGTAATCAGGGTATTTATACTAACCTCTAATAACCCAGAGCTAAG
ATCCCGGCTTCTTCTATTCTGCCTACGGATTGTTCTCAGTAATGGTGCA
AGGGATTCCCATCGCTTTGGAGCATTACTTACAATGTTTTCGCTACCAT
CAGCCACAATGCTCAATCATGTCAAATTAGCTGACCAGTCACCAGAAGC
TGATATCGAAAGGGTAGAGATCGATGGCTTTGAGGAGGGATCATTCCGC
TTAATCCCCAATGCTCGTTCAGGTATGAGCCGTGGAGAGATCAATGCCT
ATGCTGCACTTGCAGAAGATCTACCTGACACACTAAACCATGCAACACC
TTTTGTTGATTCCGAAGTCGAGGGAACTGCATGGGATGAGATTGAGACT
TTCTTAGATATGTGTTACAGTGTCCTAATGCAGGCATGGATAGTGACTT
GCAAGTGCATGACTGCGCCAGACCAACCTGCTGCTTCTATTGAGAAACG
CCTGCAAAAATATCGTCAGCAAGGCAGGATCAACCCGAGATATCTCCTG
CAACCGGAGGCTCGAAGAATAATCCAGAATGTAATCCGAAAGGGAATGG
TGGTCAGACATTTCCTCACCTTTGAACTGCAGCTTGCCCGAGCACAAAG
CCTTGTATCAAATAGGTATTATGCTATGGTAGGGGATGTTGGAAAGTAT
ATAGAGAATTGTGGAATGGGAGGCTTCTTTTTGACACTAAAATATGCAT
TAGGAACCAGATGGCCCACACTTGCTTTAGCTGCATTTTCAGGAGAGCT
AACAAAGCTAAAGTCCCTCATGGCATTATACCAGACCCTTGGTGAGCAG
GCCCGATATTTGGCCCTATTGGAGTCACCACATTTGATGGATTTTGCTG
CAGCAAACTACCCACTGCTATATAGCTATGCTATGGGAATAGGCTATGT
GTTAGATGTCAACATGAGGAACTACGCTTTCTCCAGATCATACATGAAT
AAGACATATTTCCAATTGGGAATGGAAACTGCAAGAAAACAACAGGGTG
CAGTTGACATGAGGATGGCAGAAGATCTCGGTCTAACTCAAGCCGAACG
CACCGAGATGGCAAATACACTTGCCAAATTGACCACAGCAAATCGAGGG
GCAGACACCAGGGGAGGAGTCAACCCGTTCTCATCTATCACTGGGACAA
CTCAGGTGCCCGCTGCAGCAACAGGTGACACATTCGAGAGTTACATGGC
AGCGGATCGACTGAGGCAGAGATATGCTGATGCAGGCACCCACGATGAT
GAGATGCCACCATTGGAAGAGGAGGAAGAGGACGACACATCTGCAGGTC
CACGCACTGGACTAACTCTTGAACAAGTGGCCTTGGACATCCAGAACGC
AGCAGTTGGAGCTCCCATCCATACAGATGACCTGAATGCCGCACTGGGT
GATCTTGACATCTAGACAATTCAGATCCCAATCTTAAATCGACACACCT
AATTGACCAGTTAGATGGAACTACAGTGGATTCCATAAGGTTCCTGCCT
ACCATCGGCTTTTAAGAAAAAAATAGGCCCGGACGGGTTAGCAACAAGC
GACTGCCGATGCCAATAACACAATCCACAATCTACAATGGATCCCACTG
ATCTGAGCTTCTCCCCAGATGAGATCAATAAGCTCATAGAGACAGGCCT
GAATACTGTGGAGTATTTTACTTCCCAACAAGTCACAGGAACATCCTCT
CTTGGAAAGAATACAATACCACCAGGGGTCACAGGACTACTAACCAATG
CTGCAGAGGCAAAGATCCAAGAGTCAACCAACCATCAGAAGGGTTCAGT
TGGTGGGGGCGCAAAACCAAAGAAACCGCGACCAAAAATTGCCATTGTG
CCAGCAGATGACAAAACGGTGCCCGGAAAGCCGATCCCAAACCCTCTAT
TAGGTCTGGACTCCACCCCGAGCACCCAAACTGTGCTTGATCTAAGTGG
GAAAACATTACCATCAGGATCCTATAAGGGGGTTAAACTTGCGAAATTT
GGAAAAGAAAATCTGATGACACGGTTCATCGAGGAACCCAGAGAGAATC
CTATCGCAACCAATTCCCCCATCGATTTTAAGAGGGGCAGGGATACCGG
CGGGTTCCATAGAAGGGAGTACTCAATCGGATGGGTGGGAGATGAAGTC
AAGGTCACTGAGTGGTGCAATCCATCCTGTTCTCCAATCACCGCTGCAG
CAAGGCGATTTGAATGCACTTGTCACCAGTGTCCAGTCACTTGCTCTGA
ATGTGAACGAGATACTTAATACAGTGAGAAATTTGGACTCTCGGATGAA
TCAACTGGAGACAAAAGTAGATCGCATTCTCTCATCTCAGTCTCTAATC
CAGACCATCAAGAATGACATAGTTGGACTTAAAGCAGGGATGGCTACTT
TAGAAGGAATGATTACAACTGTGAAAATCATGGACCCGGGAGTTCCCAG
TAATGTTACTGTGGAAGATGTACGCAAGAAACTAAGTAACCATGCTGTT
GTTGTGCCAGAATCATTCAATGATAGTTTCTTGACTCAATCTGAAGATG
TAATTTCACTTGATGAGTTGGCTCGACCAACTGCAACAAGTGTTAAGAA
GATTGTCAGGAAGGTTCCTCCTCAGAAGGATCTGACTGGATTGAAGATC
ACACTAGAGCAATTGGCAAAGGATTGCATCAGCAAACCGAAGATGAGGG
AAGAGTATCTCCTCAAAATCAACCAGGCTTCCAGTGAGGCTCAGCTAAT
TGACCTCAAGAAAGCAATCATCCGCAGTGCAATTTGATCAAGAAACACC
CAATTACACTACACTGGTATGACACTGTACTAACCCTGAGGGTTTTAGA
AAAAACGATTAACGATAAATAAGCCCGAACACTACACACCACCCGAGGC
AGCCATGCCATCCATCAGCATCCCCGCAGACCCCACCAATCCACGTCAA
TCAATAAAAGCGTTCCCAATTGTGATCAACCGTGATGGGGGTGAGAAAG
GTCGCTTGGTTAAACAACTACGCACAACCTACTTGAATGACCTAGATAC
TCATGAGCCACTGGTGACATTCGTAAATACTTATGGATTCATCTACGAA
CAGGATCGGGGAAATACCATTGTCGGAGAGGATCAACATGGGAAGAAAA
GAGAGGCTGTGACTGCTGCAATGGTTACCCTTGGATGTGGGCCTAATCT
ACCATCATTAGGGAATGTCCTGGGACAACTGAGTGAATTCCAGGTCATT
GTTAGGAAGACATCCAGCAAAGCGGAAGAGATGGTCTTTGAAATTGTTA
AGTATCCGAGAATATTTCGGGGTCATACATTAATCCAGAAAGGACTAGT
CTGTGTCTCCGCAGAAAAATTTGTTAAGTCACCAGGGAAAGTACAATCT
GGAATGGACTATCTCTTCATTCCGACATTTCTGTCAGTGACTTACTGTC
CAGCTGCAATCAAATTTCAGGTACCTGGCCCCATGTTGAAAATGAGATC
AAGATACACTCAGAGCTTACAACTTGAACTAATGATAAGAATCCTGTGT
AAGCCCGATTCGCCACTTATGAAGGTCCATATCCCTGACAAGGAAGGAA
GAGGATGTCTTGTATCAGTATGGTTGCATGTATGCAATATCTTCAAATC
AGGAAACAAGAATGGCAGTGAGTGGCAGGAATACTGGATGAGAAAGTGT
GCTAACATGCAACTTGAAGTGTCGATTGCAGATATGTGGGGACCAACTA
TCATAATTCATGCCAGAGGTCACATTCCCAAAAGTGCTAAGTTGTTTTT
TGGAAAGGGTGGATGGAGCTGCCATCCACTTCACGAAGTTGTTCCAAGT
GTCACTAAAACACTATGGTCCGTGGGCTGTGAGATTACAAAGGCGAAGG
CAATAATACAAGAGAGTAGCATCTCTCTTCTCGTGGAGACTACTGACAT
CATAAGTCCAAAAGTCAAAATTTCATCTAAGCATCGCCGCTTTGGGAAA
TCAAATTGGGGTCTGTTCAAGAAAACCAAATCACTGCCCAACCTGACGG
AGCTGGAATGACTGACCCCCAATCGAGACTACACCACCTCAAACTATAG
GTGGGTGGTACCTCAGTGATTAATCTCGTAAGCACTGATCGTAGGCTAC
AACACACTAATATTATCCAGATTAGAGAGCTTAATTAGCTCTGTATTAA
TAATAACACTACTATTCCAATAACTGGAATCACCAGCTTGATTTATCTC
CAAAATGATTCAAAGAAAACAAATCATATTAAGACTATCCTAAGCACGA
ACCCATATCGTCCTTCAAATCATGGGTACTATAATTCAATTTCTGGTGG
TCTCCTGTCTATTGGCAGGAGCAGGCAGTCTTGATCCAGCAGCCCTCAT
GCAAATCGGTGTCATTCCAACAAATGTCCGGCAACTTATGTATTATACT
GAGGCCTCATCAGCATTCATTGTTGTGAAGTTAATGCCTACAATTGACT
CGCCGATTAGTGGATGTAATATAACATCAATTTCAAGCTATAATGCAAC
AGTGACAAAACTCCTACAGCCGATCGGTGAGAATTTGGAGACGATTAGG
AACCAGTTGATTCCAACTCGGAGGAGACGCCGGTTTGCAGGGGTGGTGA
TTGGATTAGCTGCATTAGGAGTAGCTACTGCCGCACAGGTCACTGCCGC
AGTAGCACTAGTAAAGGCAAATGAAAATGCTGCGGCTATACTCAATCTC
AAAAATGCAATCCAAAAAACAAATGCAGCAGTTGCAGATGTGGTCCAGG
CCACACAATCACTAGGAACaGCAGTTCAAGCAGTTCAAGATCACATAAA
CAGTGTGGTAAGTCCAGCAATTACAGCAGCCAATTGTAAAGCCCAAGAT
GCTATCATTGGCTCAATCCTCAATCTCTATTTGACCGAGTTGACAACTA
TCTTCCACAATCAAATTACAAACCCTGCATTGAGTCCTATTACAATTCA
AGCTTTAAGGATCCTACTAGGGAGTACCTTGCCGACTGTGGTCGAAAAA
TCTTTCAATACCCAGATAAGTGCGGCTGAGCTTCTCTCATCAGGGTTAT
TGACAGGCCAGATTGTGGGATTAGATTTGACCTATATGCAGATGGTCAT
AAAAATTGAGCTGCCAACTTTAACTGTACAACCTGCAACCCAGATCATA
GATCTGGCCACCATTTCTGCATTCATTAACAATCAAGAAGTCATGGCCC
AATTACCAACACGTGTTATTGTGACTGGCAGCTTGATCCAAGCCTATCC
CGCATCGCAATGCACTATTACACCCAACACTGTGTACTGTAGGTATAAT
GATGCCCAAGTACTCTCAGATGATACGATGGCTTGCCTCCAAGGTAACT
TGACAAGATGCACCTTCTCTCCAGTGGTTGGGAGCTTTCTCACTCGATT
CGTGCTGTTCGATGGAATAGTTTATGCAAATTGCAGGTCGATGTTGTGC
AAGTGCATGCAGCCTGCTGCTGTGATCCTACAGCCGAGTTCATCCCCTG
TAACTGTCATTGACATGTACAAATGTGTGAGTCTGCAGCTTGACAATCT
CAGATTCACCATCACTCAATTGGCCAATGTAACCTACAATAGCACCATC
AAGCTTGAAACATCCCAGATCTTGCCTATTGATCCGTTGGATATATCCC
AGAATCTAGCTGCGGTGAATAAGAGTCTAAGTGATGCACTACAACACTT
AGCACAAAGTGACACATACCTTTCTGCAATCACATCAGCTACGACTACA
AGTGTATTATCCATAATAGCAATCTGTCTTGGATCGTTAGGTTTAATAT
TAATAATCTTGCTCAGTGTAGTTGTGTGGAAGTTATTGACCATTGTCGC
TGCTAATCGAAATAGAATGGAGAATTTTGTTTATCATAATTCAGCATTC
CACCACTCACGATCTGATCTCAGTGAGAAAAATCAACCTGCAACTCTTG
GAACAAGATAAGACAGTCATCCATTAGTAATTTTTAAGAAAAAAACGAT
AGGACCGAAACTAGTATTGAAAGAACCGTCTCGGTCAATCTAGGTAATC
GAGCTGcTACCGTCTCGGAAAGCTCAAATCATGCTGCCTGATCCGGAAG
ATCCGGAAAGCAAAAAAGCTACAAGGAGAACAGGAAACCTAATTATCTG
CTTCCTATTCATCTTCTTTCCGTTTGTAAACTTCATTGTTCCAACTCTA
AGACACTTGCTGTCCTAACACCTGCTATAGGCTATCCACTGCATCATCT
CTTTTTAAGAAAAAAATAGGCCCGGACGGGTTAGCAACAAGCGACTGCC
GGTGCCAACAACACAATCCACAATCTACAGTCGACGCGGCCGCCACCat
gttcgtcttcctggtcctgctgcctctggtctcctcacagtgcgtcaat
ctgATCactcggactcagTCCtatactaatagcttcaccagaggcgtgt
actatcctgacaaggtgtttagaagctccgtgctgcactctacacagga
tctgtttctgccattctttagcaacgtgacctggttccacgccatccac
gtgagcggcaccaatggcacaaagcggttcgacaatcccgtgctgcctt
ttaacgatggcgtgtacttcgcctctaccgagaagagcaacatcatcag
aggctggatctttggcaccacactggactccaagacacagtctctgctg
atcgtgaacaatgccaccaacgtggtcatcaaggtgtgcgagttccagt
tttgtaatgatcccttcctgGACgtgtactatcacaagaacaataagag
ctggatggagtccgagtttagagtgtattctagcgccaacaactgcaca
tttgagtacgtgagccagcctttcctgatggacctggagggcaagcagg
gcaatttcaagaacctgagggagttcgtgtttaagaatatcgacggcta
cttcaaaatctactctaagcacacccccatcaacctgGGCcgcgacctg
cctcagggcttcagcgccctggagcccctggtggatctgcctatcggca
tcaacatcacccggtttcagacactgctggccctgcacagaagctacct
gacacccggcgactcctctagcggatggaccgccggcgctgccgcctac
tatgtgggctacctccagccccggaccttcctgctgaagtacaacgaga
atggcaccatcacagacgcagtggattgcgccctggaccccctgagcga
gacaaagtgtacactgaagtcctttaccgtggagaagggcatctatcag
acatccaatttcagggtgcagccaaccgagtctatcgtgcgctttccta
atatcacaaacctgtgcccatttGACgaggtgttcaacgcaacccgctt
cgccagcgtgtacgcctggaataggaagcggatcagcaactgcgtggcc
gactatagcgtgctgtacaacCTGgccCCCttcTTCGCCtttaagtgct
atggcgtgtcccccacaaagctgaatgacctgtgctttaccaacgtcta
cgccgattctttcgtgatcaggggcAACgaggtgTCCcagatcgccccc
ggccagacaggcAATatcgcagactacaattataagctgccagacgatt
tcaccggctgcgtgatcgcctggaacagcaacAAGctggattccaaagt
gggcggcaactacaattatctgtaccggctgtttagaaagagcaatctg
aagcccttcgagagggacatctctacagaaatctaccaggccggcAATA
AGccttgcaatggcgtgGCCggctttaactgttatttcccactcCGCtc
ctacggcttcCGCcccacaTATggcgtgggcCACcagccttaccgcgtg
gtggtgctgagctttgagctgctgcacgccccagcaacagtgtgcggcc
ccaagaagtccaccaatctggtgaagaacaagtgcgtgaacttcaactt
caacggcctgaccggcacaggcgtgctgaccgagtccaacaagaagttc
ctgccatttcagcagttcggcagggacatcgcagataccacagacgccg
tgcgcgacccacagaccctggagatcctggacatcacaccctgctcttt
cggcggcgtgagcgtgatcacacccggcaccaatacaagcaaccaggtg
gccgtgctgtatcagGGCgtgaattgtaccgaggtgcccgtggctatcc
acgccgatcagctgaccccaacatggcgggtgtacagcaccggctccaa
cgtcttccagacaagagccggatgcctgatcggagcagagTACgtgaac
aattcctatgagtgcgacatcccaatcggcgccggcatctgtgcctctt
accagacccagacaAAGtctCACagaagagcccggagcgtggcctccca
gtctatcatcgcctataccatgtccctgggcgccgagaacagcgtggcc
tactctaacaatagcatcgccatcccaaccaacttcacaatctctgtga
ccacagagatcctgcccgtgtccatgaccaagacatctgtggactgcac
aatgtatatctgtggcgattctaccgagtgcagcaacctgctgctccag
tacggcagcttttgtacccagctgAAGagagccctgacaggcatcgccg
tggagcaggataagaacacacaggaggtgttcgcccaggtgaagcaaat
ctacaagaccccccctatcaagTACtttggcggcttcaatttttcccag
atcctgcctgatccatccaagccttctaagcggagctttatcgaggacc
tgctgttcaacaaggtgaccctggccgatgccggcttcatcaagcagta
tggcgattgcctgggcgacatcgcagccagggacctgatctgcgcccag
aagtttaatggcctgaccgtgctgccacccctgctgacagatgagatga
tcgcacagtacacaagcgccctgctggccggcaccatcacatccggatg
gaccttcggcgcaggagccgccctccagatcccctttgccatgcagatg
gcctataggttcaacggcatcggcgtgacccagaatgtgctgtacgaga
accagaagctgatcgccaatcagtttaactccgccatcggcaagatcca
ggacagcctgtcctctacagccagcgccctgggcaagctccaggatgtg
gtgaatCACaacgcccaggccctgaataccctggtgaagcagctgagca
gcAAGttcggcgccatctctagcgtgctgaatgacatcctgagccggct
ggacaaggtggaggcagaggtgcagatcgaccggctgatcaccggccgg
ctccagagcctccagacctatgtgacacagcagctgatcagggccgccg
agatcagggccagcgccaatctggcagcaaccaagatgtccgagtgcgt
gctgggccagtctaagagagtggacttttgtggcaagggctatcacctg
atgtccttccctcagtctgccccacacggcgtggtgtttctgcacgtga
cctacgtgcccgcccaggagaagaacttcaccacagcccctgccatctg
ccacgatggcaaggcccactttccaagggagggcgtgttcgtgtccaac
ggcacccactggtttgtgacacagcgcaatttctacgagccccagatca
tcaccacagacaacaccttcgtgagcggcaactgtgacgtggtcatcgg
catcgtgaacaataccgtgtatgatccactccagcccgagctggacagc
tttaaggaggagctggataagtatttcaagaatcacacctcccctgacg
tggatctgggcgacatcagcggcatcaatgcctccgtggtgaacatcca
gaaggagatcgaccgcctgaacgaggtggctaagaatctgaacgagagc
ctgatcgacctccaggagctgggcaagtatgagcagtacatcaagtggc
cctggtacatctggctgggcttcatcgccggcctgatcgccatcgtgat
ggtgaccatcatgctgtgctgtatgacatcctgctgttcttgcctgaag
ggctgctgtagctgtggctcctgctgtAAGTTATTGACCATTGTCGTTG
CTAATCGAAATAGAATGGAGAATTTTGTTTATCATAAATGAGCTAGCCT
CCTGCCATACTTCCTACTCACATCATATCTATTTTAAAGAAAAAATAGG
CCCGAACACTAATCGTGCCGGCAGTGCCACTGCACACACAACACTACAC
ATACAATACACTACAATGGTTGCAGAAGATGCCCCTGTTAGGGGCACTT
GCCGAGTATTATTTCGAACAACAACTTTAATTTTTCTATGCACACTATT
AGCATTAAGCATCTCTATCCTTTATGAGAGTTTAATAACCCAAAAGCAA
ATCATGAGCCAAGCAGGCTCAACTGGATCTAATTCTAGATTAGGAAGTA
TCACTGATCTTCTTAATAATATTCTCTCTGTCGCAAATCAGATTATATA
TAACTCTGCAGTCGCTCTACCTCTACAATTGGACACTCTTGAATCAACA
CTCCTTACAGCCATTAAGTCTCTTCAAACAAGTGACAAGCTAGAACAGA
ACTGCTCGTGGGGTGCTGCACTGATTAATGATAATAGATACATTAATGG
CATCAATCAGTTCTATTTCTCAATTGCTGAGGGTCGCAAGCTGACACTT
GGCCCACTTCTTAATATACCTAGTTTCATTCCAACTGCCACGACACCAG
AGGGCTGCACCAGGATCCCATCATTCTCGCTCACCAAGACACACTGGTG
TTATACACACAATGTTATCCTGAATGGATGCCAGGATCATGTATCCTCA
AATCAATTTGTTTCCATGGGAATCATTGAACCCACTTCTGCCGGGTTTC
CATCCTTTCGAACCCTAAAGACTCTATATCTCAGCGATGGGGTCAATCG
TAAGAGCTGCTCTATCAGTACAGTTCCGGGGGGTTGTATGATGTACTGT
TTCGTCTCTACTCAACCAGAGAGGGATGACTACTTTTCTACCGCTCCTC
CAGAACAACGAATTATTATAATGTACTATAATGATACAATCGTGGAGCG
CATAATTAATCCACCCGGGGTACTAGATGTATGGGCAACATTGAACCCA
GGAACAGGAAGCGGGGTATATTATTTAGGTTGGGTACTCTTTCCAATAT
ATGGCGGCGTGATTAAAGATACGAGTTTATGGAATAATCAAGCAAATAA
ATACTTTATCCCCCAGATGGTTGCTGCTCTCTGCTCACAAAACCAGGCA
ACTCAAGTCCAAAATGCTAAGTCATCATACTATAGCAGCTGGTTTGGCA
ATCGAATGATTCAGTCTGGGATCCTGGCATGTCCTCTTCAACAGGATCT
AACCAATGAGTGTTTAGTTCTGCCCTTTTCTAATGATCAGGTGCTTATG
GGTGCTGAAGGGAGATTATACATGTATGGTGACTCGGTGTATTACTACC
AAAGAAGCAATAGTTGGTGGCCTATGACCATGCTGTATAAGGTAACCAT
AACATTCACTAATGGTCAGCCATCTGCTATATCAGCTCAGAATGTGCCC
ACACAGCAGGTCCCTAGACCTGGGACAGGAAGCTGCTCTGCAACAAATA
GATGTCCCGGTTTTTGCTTGAAAGGAGTGTATGCTGATGCCTGGTTACT
GACCAACCCTTCGTCTACCAGTACATTTGGATCAGAAGCAACCTTCACT
GGTTCTTATCTCAACGCAGCAACTCAGCGTATCAATCCGACGATGTATA
TCGCGAACAACACACAGATCATAAGCTCACAGCAATTTGGATCAAGCGG
TCAAGAAGCAGCATATGGCCACACAACTTGTTTTAGGGACACAGGCTCT
GTTATGGTATACTGTATCTATATTATTGAATTGTCCTCATCTCTCTTAG
GACAATTTCAGATTGTCCCATTTATCCGTCAGGTGACACTATCCTAAAG
GCAGAAGCCTCCAGGTCTGACCCAGCCAATCAAAGCATTATACCAGACC
ATGGAATGCATACCAAACATTATTGACACTAATGACACACAAAATTGGT
TTTAAGAAAAACCAAGAGAACAATAGGCCAGAATGGCTGGGTCTCGGGA
GATATTACTCCCTGAAGTCCATCTCAATTCACCAATTGTAAAGCATAAG
CTATACTATTACATTCTACTTGGAAACCTCCCAAATGAGATCGACATTG
ACGATTTAGGTCCATTACATAATCAAAATTGGAATCAAATAGCACATGA
AGAGTCTAACTTAGCCCAACGCTTGGTAAATGTAAGAAATTTTCTAATT
ACCCACATCCCTGATCTTAGAAAGGGCCATTGGCAAGAGTATGTCAATG
TAATACTGTGGCCGCGAATTCTTCCCTTGATCCCGGATTTTAAAATCAA
TGACCAATTGCCTCTACTCAAAAATTGGGACAAGTTAGTTAAAGAATCA
TGTTCAGTAATCAATGCGGGTACTTCCCAGTGCATTCAGAATCTCAGCT
ATGGACTGACAGGTCGTGGGAACCTCTTTACACGATCACGTGAACTCTC
TGGTGACCGCAGGGATATTGATCTTAAGACGGTTGTGGCAGCATGGCAT
GACTCAGACTGGAAAAGAATAAGTGATTTTTGGATTATGATCAAATTCC
AGATGAGACAATTAATTGTTAGGCAAACAGATCATAATGATCCTGATTT
AATCACGTATATCGAAAATAGAGAAGGCATAATCATCATAACCCCTGAA
CTGGTAGCATTATTTAACACTGAGAATCATACACTAACATACATGACCT
TTGAAATTGTACTGATGGTTTCAGATATGTACGAAGGTCGTCACAACAT
TTTATCACTATGCACAGTTAGCACTTACCTGAATCCTCTGAAGAAAAGA
ATAACATATTTATTGAGCCTTGTAGATAACTTAGCTTTTCAGATAGGTG
ATGCTGTATATAACATAATTGCTTTGCTAGAATCCTTTGTATATGCACA
GTTGCAAATGTCAGATCCCATCCCAGAACTCAGAGGACAATTCCATGCA
TTCGTATGTTCTGAGATTCTTGATGCACTAAGGGGAACTAATAGTTTCA
CCCAGGATGAATCAAGAACTGTGACAACCAATTTGATATCCCCATTCCA
AGATCTGACCCCAGATCTTACGGCTGAATTGCTCTGTATAATGAGGCTT
TGGGGACACCCCATGCTCACCGCCAGTCAAGCTGCGGGAAAGGTACGCG
AGTCCATGTGTGCTGGAAAAGTATTAGACTTTCCCACCATTATGAAAAC
ACTAGCCTTTTTCCATACTATTCTGATCAATGGATACAGGAGGAAGCAT
CATGGAGTATGGCCACCCTTAAACTTACCGGGTAATGCTTCAAAGGGTC
TCACGGAACTTATGAATGACAATACTGAGATAAGCTATGAATTCACACT
TAAGCATTGGAAGGAAATCTCTCTTATAAAATTCAAGAAATGTTTTGAT
GCAGACGCAGGTGAGGAACTCAGTATATTTATGAAAGATAAAGCAATTA
GTGCCCCAAAACAAGATTGGATGAGTGTGTTTAGAAGAAGCCTAATCAA
ACAGCGCCATCAGCATCATCAGGTCCCCCTACCAAATCCATTCAATCGA
CGGCTATTGCTAAACTTTCTCGGAGATGACAAATTCGACCCGAATGTGG
AGCTACAGTATGTAACATCAGGTGAGTATCTACATGATGACACGTTTTG
TGCATCATATTCACTAAAAGAGAAGGAAATTAAACCTGATGGTCGAATT
TTTGCAAAGTTGACTAAGAGAATGAGATCATGTCAAGTTATAGCAGAAT
CTCTTTTAGCGAACCATGCTGGGAAGTTAATGAAAGAGAATGGTGTTGT
GATGAATCAGCTATCATTAACAAAATCACTATTAACAATGAGTCAGATT
GGAATAATATCCGAGAAAGCTAGAAAATCGACTCGAGATAACATAAATC
AACCTGGTTTCCAGAATATCCAGAGAAATAAATCACATCACTCCAAGCA
AGTCAATCAGCGAGATCCAAGTGATGACTTTGAATTGGCAGCATCTTTT
TTAACTACTGATCTCAAAAAATATTGTTTACAATGGAGGTACCAGACAA
TTATCCCATTTGCTCAATCATTAAACAGAATGTATGGTTATCCTCATCT
CTTTGAGTGGATTCACTTACGGCTAATGCGTAGTACACTTTACGTGGGG
GATCCCTTCAACCCACCAGCAGATACCAGTCAATTTGATCTAGATAAAG
TAATTAATGGAGATATCTTCATTGTATCACCCAGAGGTGGAATTGAAGG
GCTATGTCAAAAGGCTTGGACAATGATATCTATCTCTGTGATAATTCTA
TCTGCCACAGAGTCTGGCACACGAGTAATGAGTATGGTGCAGGGAGATA
ATCAAGCAATTGCTGTCACCACACGAGTACCAAGGAGCCTGCCGACTCT
TGAGAAAAAGACTATTGCTTTTAGATCTTGTAATCTATTCTTTGAGAGG
TTAAAATGTAATAATTTTGGATTAGGTCACCATTTGAAAGAACAAGAGA
CTATCATTAGTTCTCACTTCTTTGTTTATAGCAAGAGAATATTCTATCA
GGGGAGGATTCTAACGCAAGCCTTAAAAAATGCTAGTAAGCTCTGCTTG
ACAGCTGATGTCCTAGGAGAATGTACCCAATCATCATGTTCTAATCTTG
CAACTACTGTCATGAGGTTAACTGAGAATGGTGTTGAAAAAGATATCTG
TTTCTACTTGAATATCTATATGACCATCAAACAGCTCTCCTATGATATC
ATCTTCCCTCAAGTGTCAATTCCTGGAGATCAGATCACATTAGAATACA
TAAATAATCCACACCTGGTATCACGATTGGCTCTTCTGCCATCCCAGCT
AGGAGGTCTAAACTACCTGTCATGCAGTAGGCTGTTCAATCGAAACATA
GGCGACCCGGTGGTTTCCGCAGTTGCAGATCTTAAGAGATTAATTAAAT
CAGGATGTATGGATTACTGGATCCTTTATAACTTATTAGGGAGAAAACC
GGGAAACGGCTCATGGGCTACTTTAGCAGCTGACCCGTACTCAATCAAT
ATAGAGTATCAATACCCCCCAACTACAGCTCTTAAGAGGCACACCCAAC
AAGCTCTGATGGAACTCAGTACGAATCCAATGTTACGTGGCATATTCTC
TGACAATGCACAGGCAGAAGAAAATAATCTTGCTAGATTTCTCCTGGAT
AGGGAGGTGATCTTTCCGCGTGTAGCTCACATCATCATTGAGCAAACCA
GTGTCGGGAGGAGAAAACAGATTCAAGGATATTTGGATTCAACTAGATC
GATAATGAGTAAATCACTAGAAATTAAGCCCTTGTCCAATAGGAAGCTT
AATGAAATACTGGATTACAACATCAATTACCTAGCTTACAATTTGGCAT
TACTCAAGAATGCTATTGAACCTCCGACTTATTTGAAAGCAATGACTCT
TGAAACATGTAGCATCGACATTGCAAGGAGCCTCCGGAAGCTCTCCTGG
GCCCCACTCTTGGGTGGGAGAAATCTTGAAGGATTAGAGACGCCAGATC
CCATTGAAATTACTGCAGGAGCATTAATTGTTGGATCGGGCTACTGTGA
ACAGTGTGCTGCAGGAGACAATCGATTCACATGGTTTTTCTTGCCATCT
GGTATCGAGATAGGAGGGGATCCCCGTGATAATCCTCCTATCCGTGTAC
CGTACATTGGCTCCAGGACTGATGAGAGGAGGGTAGCCTCAATGGCATA
CATCAGGGGTGCCTCGAGTAGCCTAAAAGCAGTTCTTAGACTGGCGGGA
GTGTACATCTGGGCATTCGGAGATACTCTGGAGAATTGGATAGATGCAC
TGGATTTGTCTCACACTAGAGTTAACATCACACTTGAACAGCTGCAATC
CCTCACCCCACTTCCAACCTCTGCCAATCTAACCCATCGGTTGGATGAT
GGCACAACTACCCTAAAGTTTACTCCTGCGAGCTCTTACACCTTTTCAA
GTTTCACTCATATATCAAATGATGAGCAATACCTGACAATTAATGACAA
AACTGCAGATTCAAATATAATCTACCAACAGTTAATGATCACTGGACTC
GGAATTTTAGAAACATGGAATAATCCCCCAATCAATAGAACATTCGAAG
AATCTACCCTACATTTGCACACTGGTGCATCATGTTGTGTCCGACCTGT
GGACTCCTGCATCATCTCAGAAGCATTAACAGTCAAGCCACATATTACA
GTACCGTACAGCAATAAATTTGTATTTGATGAAGACCCGCTATCTGAAT
ATGAGACTGCAAAACTGGAATCGTTATCATTTCAAGCCCAATTAGGCAA
CATTGATGCTGTAGATATGACAGGTAAATTAACATTATTGTCCCAATTC
ACTGCAAGGCAGATTATTAATGCAATCACTGGACTCGATGAGTCTGTCT
CTCTTACTAATGATGCCATTGTTGCATCAGACTATGTCTCCAATTGGAT
TAGTGAATGCATGTATACCAAATTAGATGAATTATTTATGTATTGTGGG
TGGGAACTACTATTGGAACTATCCTATCAAATGTATTATCTGAGGGTAG
TTGGGTGGAGTAATATAGTGGATTATTCTTACATGATCTTGAGAAGAAT
CCCGGGTGCAGCATTAAACAATCTGGCATCTACATTAAGTCATCCAAAA
CTTTTCCGACGAGCTATCAACCTAGATATAGTTGCCCCCTTAAATGCTC
CTCATTTTGCATCTCTGGACTACATCAAGATGAGTGTGGATGCAATACT
CTGGGGCTGTAAAAGAGTCATCAATGTGCTCTCCAATGGAGGGGACTTA
GAATTAGTTGTGACATCTGAAGATAGCCTTATTCTCAGTGACCGATCCA
TGAATCTCATTGCAAGGAAATTAACTTTATTATCACTGATTCACCATAA
TGGTTTGGAACTACCAAAGATTAAGGGGTTCTCTCCTGATGAGAAGTGT
TTCGCTTTGACAGAATTTTTGAGGAAAGTGGTGAACTCAGGGTTGAGTT
CAATAGAGAACCTATCAAATTTTATGTACAATGTGGAGAACCCACGGCT
TGCAGCATTCGCCAGCAACAATTACTACCTGACCAGAAAATTATTGAAT
TCAATACGAGATACTGAGTCGGGTCAAGTAGCAGTCACCTCATATTATG
AATCATTAGAATATATTGATAGTCTTAAGCTAACCCCACATGTGCCTGG
TACCTCATGCATTGAGGATGATAGTCTATGTACAAATGATTACATAATC
TGGATCATAGAGTCTAATGCAAACTTGGAGAAGTATCCAATTCCAAATA
GCCCTGAGGATGATTCCAATTTCCATAACTTTAAGTTGAATGCTCCATC
GCACCATACCTTACGCCCATTAGGGTTGTCATCAACTGCTTGGTATAAG
GGTATAAGCTGTTGCAGGTACCTTGAGCGATTAAAGCTACCACAAGGTG
ATCATTTATATATTGCAGAAGGTAGTGGTGCCAGTATGACAATCATAGA
ATACCTATTCCCAGGAAGAAAGATATATTACAATTCTTTATTTAGTAGT
GGTGACAATCCCCCACAAAGAAATTATGCACCAATGCCTACTCAGTTCA
TTGAGAGTGTCCCATACAAGCTCTGGCAAGCACACACAGATCAATATCC
CGAGATTTTTGAGGACTTCATCCCTCTATGGAACGGAAATGCCGCCATG
ACTGACATAGGAATGACAGCTTGTGTAGAATTCATCATCAATCGAGTCG
GCCCAAGGACTTGCAGTTTAGTACATGTAGATTTGGAATCAAGTGCAAG
CTTAAATCAACAATGCCTGTCAAAGCCGATAATTAATGCTATCATCACT
GCTACAACTGTTTTGTGCCCTCATGGGGTGCTTATTCTGAAATATAGTT
GGTTGCCATTTACTAGATTTAGTACTTTGATCACTTTCTTATGGTGCTA
CTTTGAGAGAATCACTGTTCTTAGGAGCACATATTCTGGTCCAGCTAAT
CATGAGGTTTATTTAATTTGTATCCTTGCCAACAACTTTGCATTCCAGA
CTGTCTCGCAGGCAACAGGAATGGCGATGACTTTAACCGATCAAGGGTT
TACTTTGATATCACCTGAAAGAATAAATCAGTATTGGGATGGTCACTTG
AAGCAAGAACGTATCGTAGCAGAAGCAATTGATAAGGTGGTTCTAGGAG
AAGATGCTCTATTCAATTCGAGTGATAATGAATTAATTCTCAAATGTGG
AGGGACACCAAATGCACGGAATCTTATCGATATCGAGCCAGTCGCAACT
TTCATAGAATTTGAACAACTGATCTGCACAATGTTGACAACCCACTTGA
AGGAAATAATTGATATAACAAGGTCTGGAACCCAGGATTATGAAAGTTT
ATTACTCACTCCTTACAATTTAGGTCTTCTTGGTAAAATCAGTACGATA
GTGAGATTATTAACAGAAAGGATTCTAAATCATACTATCAGGAATTGGT
TGATCCTCCCACCTTCGCTCCGGATGATCGTGAAGCAGGACTTGGAATT
CGGCATATTCAGGATTACTTCCATCCTCAATTCTGATCGGTTCCTGAAG
CTTTCTCCAAATAGGAAATACTTGATTACACAATTAACTGCAGGCTACA
TTAGGAAATTGATTGAGGGGGATTGTAATATCGATCTAACCAGACCTAT
CCAAAAACAAATCTGGAAAGCATTAGGTTGTGTAGTCTATTGTCACGAT
CCAGTAGATCAAAGGGAATCAACAGAGTTTATTGATATAAATATTAATG
AAGAAATAGACCGCGGGATCGATGGCGAGGAAATCTAAATATATCAAGA
ATCAGAATTAGTTTAAGAAAAAAGAAGAGGATTAATCTTGGTTTTCCCC
TTGGT.

19. CVL108

The nucleic acid sequence for CVL108 is:

(SEQ ID NO: 78)
ACCAGGGGGAAAACGAAGTGGTGACTCAAATCATCGAAGACCCTCGAGATTACATAGGTCCGGAACC
TATGGCCTTCGTGACCGACCTCGAGTCAGAGTAGTTCAATAAGGACCTATCAAGTTTGGGCAATTTTTC
GTCCCCGACACAAAAATGTCATCCGTGCTTAAAGCATATGAGCGATTCACACTCACTCAAGAACTGCA
AGATCAGAGTGAGGAAGGTACAATCCCACCTACAACACTAAAACCGGTAATCAGGGTATTTATACTAA
CCTCTAATAACCCAGAGCTAAGATCCCGGCTTCTTCTATTCTGCCTACGGATTGTTCTCAGTAATGGTG
CAAGGGATTCCCATCGCTTTGGAGCATTACTTACAATGTTTTCGCTACCATCAGCCACAATGCTCAATC
ATGTCAAATTAGCTGACCAGTCACCAGAAGCTGATATCGAAAGGGTAGAGATCGATGGCTTTGAGGA
GGGATCATTCCGCTTAATCCCCAATGCTCGTTCAGGTATGAGCCGTGGAGAGATCAATGCCTATGCTG
CACTTGCAGAAGATCTACCTGACACACTAAACCATGCAACACCTTTTGTTGATTCCGAAGTCGAGGGA
ACTGCATGGGATGAGATTGAGACTTTCTTAGATATGTGTTACAGTGTCCTAATGCAGGCATGGATAGT
GACTTGCAAGTGCATGACTGCGCCAGACCAACCTGCTGCTTCTATTGAGAAACGCCTGCAAAAATATC
GTCAGCAAGGCAGGATCAACCCGAGATATCTCCTGCAACCGGAGGCTCGAAGAATAATCCAGAATGT
AATCCGAAAGGGAATGGTGGTCAGACATTTCCTCACCTTTGAACTGCAGCTTGCCCGAGCACAAAGCC
TTGTATCAAATAGGTATTATGCTATGGTAGGGGATGTTGGAAAGTATATAGAGAATTGTGGAATGGGA
GGCTTCTTTTTGACACTAAAATATGCATTAGGAACCAGATGGCCCACACTTGCTTTAGCTGCATTTTCA
GGAGAGCTAACAAAGCTAAAGTCCCTCATGGCATTATACCAGACCCTTGGTGAGCAGGCCCGATATTT
GGCCCTATTGGAGTCACCACATTTGATGGATTTTGCTGCAGCAAACTACCCACTGCTATATAGCTATGC
TATGGGAATAGGCTATGTGTTAGATGTCAACATGAGGAACTACGCTTTCTCCAGATCATACATGAATA
AGACATATTTCCAATTGGGAATGGAAACTGCAAGAAAACAACAGGGTGCAGTTGACATGAGGATGGC
AGAAGATCTCGGTCTAACTCAAGCCGAACGCACCGAGATGGCAAATACACTTGCCAAATTGACCACA
GCAAATCGAGGGGCAGACACCAGGGGAGGAGTCAACCCGTTCTCATCTATCACTGGGACAACTCAGG
TGCCCGCTGCAGCAACAGGTGACACATTCGAGAGTTACATGGCAGCGGATCGACTGAGGCAGAGATA
TGCTGATGCAGGCACCCACGATGATGAGATGCCACCATTGGAAGAGGAGGAAGAGGACGACACATCT
GCAGGTCCACGCACTGGACTAACTCTTGAACAAGTGGCCTTGGACATCCAGAACGCAGCAGTTGGAGC
TCCCATCCATACAGATGACCTGAATGCCGCACTGGGTGATCTTGACATCTAGACAATTCAGATCCCAA
TCTTAAATCGACACACCTAATTGACCAGTTAGATGGAACTACAGTGGATTCCATAAGGTTCCTGCCTAC
CATCGGCTTTTAAGAAAAAAATAGGCCCGGACGGGTTAGCAACAAGCGACTGCCGATGCCAATAACA
CAATCCACAATCTACAATGGATCCCACTGATCTGAGCTTCTCCCCAGATGAGATCAATAAGCTCATAG
AGACAGGCCTGAATACTGTGGAGTATTTTACTTCCCAACAAGTCACAGGAACATCCTCTCTTGGAAAG
AATACAATACCACCAGGGGTCACAGGACTACTAACCAATGCTGCAGAGGCAAAGATCCAAGAGTCAA
CCAACCATCAGAAGGGTTCAGTTGGTGGGGGCGCAAAACCAAAGAAACCGCGACCAAAAATTGCCAT
TGTGCCAGCAGATGACAAAACGGTGCCCGGAAAGCCGATCCCAAACCCTCTATTAGGTCTGGACTCCA
CCCCGAGCACCCAAACTGTGCTTGATCTAAGTGGGAAAACATTACCATCAGGATCCTATAAGGGGGTT
AAACTTGCGAAATTTGGAAAAGAAAATCTGATGACACGGTTCATCGAGGAACCCAGAGAGAATCCTA
TCGCAACCAATTCCCCCATCGATTTTAAGAGGGGCAGGGATACCGGCGGGTTCCATAGAAGGGAGTAC
TCAATCGGATGGGTGGGAGATGAAGTCAAGGTCACTGAGTGGTGCAATCCATCCTGTTCTCCAATCAC
CGCTGCAGCAAGGCGATTTGAATGCACTTGTCACCAGTGTCCAGTCACTTGCTCTGAATGTGAACGAG
ATACTTAATACAGTGAGAAATTTGGACTCTCGGATGAATCAACTGGAGACAAAAGTAGATCGCATTCT
CTCATCTCAGTCTCTAATCCAGACCATCAAGAATGACATAGTTGGACTTAAAGCAGGGATGGCTACTT
TAGAAGGAATGATTACAACTGTGAAAATCATGGACCCGGGAGTTCCCAGTAATGTTACTGTGGAAGAT
GTACGCAAGAAACTAAGTAACCATGCTGTTGTTGTGCCAGAATCATTCAATGATAGTTTCTTGACTCAA
TCTGAAGATGTAATTTCACTTGATGAGTTGGCTCGACCAACTGCAACAAGTGTTAAGAAGATTGTCAG
GAAGGTTCCTCCTCAGAAGGATCTGACTGGATTGAAGATCACACTAGAGCAATTGGCAAAGGATTGCA
TCAGCAAACCGAAGATGAGGGAAGAGTATCTCCTCAAAATCAACCAGGCTTCCAGTGAGGCTCAGCT
AATTGACCTCAAGAAAGCAATCATCCGCAGTGCAATTTGATCAAGAAACACCCAATTACACTACACTG
GTATGACACTGTACTAACCCTGAGGGTTTTAGAAAAAACGATTAACGATAAATAAGCCCGAACACTAC
ACACCACCCGAGGCAGCCATGCCATCCATCAGCATCCCCGCAGACCCCACCAATCCACGTCAATCAAT
AAAAGCGTTCCCAATTGTGATCAACCGTGATGGGGGTGAGAAAGGTCGCTTGGTTAAACAACTACGCA
CAACCTACTTGAATGACCTAGATACTCATGAGCCACTGGTGACATTCGTAAATACTTATGGATTCATCT
ACGAACAGGATCGGGGAAATACCATTGTCGGAGAGGATCAACATGGGAAGAAAAGAGAGGCTGTGA
CTGCTGCAATGGTTACCCTTGGATGTGGGCCTAATCTACCATCATTAGGGAATGTCCTGGGACAACTG
AGTGAATTCCAGGTCATTGTTAGGAAGACATCCAGCAAAGCGGAAGAGATGGTCTTTGAAATTGTTAA
GTATCCGAGAATATTTCGGGGTCATACATTAATCCAGAAAGGACTAGTCTGTGTCTCCGCAGAAAAAT
TTGTTAAGTCACCAGGGAAAGTACAATCTGGAATGGACTATCTCTTCATTCCGACATTTCTGTCAGTGA
CTTACTGTCCAGCTGCAATCAAATTTCAGGTACCTGGCCCCATGTTGAAAATGAGATCAAGATACACT
CAGAGCTTACAACTTGAACTAATGATAAGAATCCTGTGTAAGCCCGATTCGCCACTTATGAAGGTCCA
TATCCCTGACAAGGAAGGAAGAGGATGTCTTGTATCAGTATGGTTGCATGTATGCAATATCTTCAAAT
CAGGAAACAAGAATGGCAGTGAGTGGCAGGAATACTGGATGAGAAAGTGTGCTAACATGCAACTTGA
AGTGTCGATTGCAGATATGTGGGGACCAACTATCATAATTCATGCCAGAGGTCACATTCCCAAAAGTG
CTAAGTTGTTTTTTGGAAAGGGTGGATGGAGCTGCCATCCACTTCACGAAGTTGTTCCAAGTGTCACTA
AAACACTATGGTCCGTGGGCTGTGAGATTACAAAGGCGAAGGCAATAATACAAGAGAGTAGCATCTC
TCTTCTCGTGGAGACTACTGACATCATAAGTCCAAAAGTCAAAATTTCATCTAAGCATCGCCGCTTTGG
GAAATCAAATTGGGGTCTGTTCAAGAAAACCAAATCACTGCCCAACCTGACGGAGCTGGAATGACTG
ACCCCCAATCGAGACTACACCACCTCAAACTATAGGTGGGTGGTACCTCAGTGATTAATCTCGTAAGC
ACTGATCGTAGGCTACAACACACTAATATTATCCAGATTAGAGAGCTTAATTAGCTCTGTATTAATAAT
AACACTACTATTCCAATAACTGGAATCACCAGCTTGATTTATCTCCAAAATGATTCAAAGAAAACAAA
TCATATTAAGACTATCCTAAGCACGAACCCATATCGTCCTTCAAATCATGGGTACTATAATTCAATTTC
TGGTGGTCTCCTGTCTATTGGCAGGAGCAGGCAGTCTTGATCCAGCAGCCCTCATGCAAATCGGTGTC
ATTCCAACAAATGTCCGGCAACTTATGTATTATACTGAGGCCTCATCAGCATTCATTGTTGTGAAGTTA
ATGCCTACAATTGACTCGCCGATTAGTGGATGTAATATAACATCAATTTCAAGCTATAATGCAACAGT
GACAAAACTCCTACAGCCGATCGGTGAGAATTTGGAGACGATTAGGAACCAGTTGATTCCAACTCGGA
GGAGACGCCGGTTTGCAGGGGTGGTGATTGGATTAGCTGCATTAGGAGTAGCTACTGCCGCACAGGTC
ACTGCCGCAGTAGCACTAGTAAAGGCAAATGAAAATGCTGCGGCTATACTCAATCTCAAAAATGCAAT
CCAAAAAACAAATGCAGCAGTTGCAGATGTGGTCCAGGCCACACAATCACTAGGAACaGCAGTTCAAG
CAGTTCAAGATCACATAAACAGTGTGGTAAGTCCAGCAATTACAGCAGCCAATTGTAAAGCCCAAGAT
GCTATCATTGGCTCAATCCTCAATCTCTATTTGACCGAGTTGACAACTATCTTCCACAATCAAATTACA
AACCCTGCATTGAGTCCTATTACAATTCAAGCTTTAAGGATCCTACTAGGGAGTACCTTGCCGACTGTG
GTCGAAAAATCTTTCAATACCCAGATAAGTGCGGCTGAGCTTCTCTCATCAGGGTTATTGACAGGCCA
GATTGTGGGATTAGATTTGACCTATATGCAGATGGTCATAAAAATTGAGCTGCCAACTTTAACTGTAC
AACCTGCAACCCAGATCATAGATCTGGCCACCATTTCTGCATTCATTAACAATCAAGAAGTCATGGCC
CAATTACCAACACGTGTTATTGTGACTGGCAGCTTGATCCAAGCCTATCCCGCATCGCAATGCACTATT
ACACCCAACACTGTGTACTGTAGGTATAATGATGCCCAAGTACTCTCAGATGATACGATGGCTTGCCT
CCAAGGTAACTTGACAAGATGCACCTTCTCTCCAGTGGTTGGGAGCTTTCTCACTCGATTCGTGCTGTT
CGATGGAATAGTTTATGCAAATTGCAGGTCGATGTTGTGCAAGTGCATGCAGCCTGCTGCTGTGATCCT
ACAGCCGAGTTCATCCCCTGTAACTGTCATTGACATGTACAAATGTGTGAGTCTGCAGCTTGACAATCT
CAGATTCACCATCACTCAATTGGCCAATGTAACCTACAATAGCACCATCAAGCTTGAAACATCCCAGA
TCTTGCCTATTGATCCGTTGGATATATCCCAGAATCTAGCTGCGGTGAATAAGAGTCTAAGTGATGCAC
TACAACACTTAGCACAAAGTGACACATACCTTTCTGCAATCACATCAGCTACGACTACAAGTGTATTA
TCCATAATAGCAATCTGTCTTGGATCGTTAGGTTTAATATTAATAATCTTGCTCAGTGTAGTTGTGTGG
AAGTTATTGACCATTGTCGCTGCTAATCGAAATAGAATGGAGAATTTTGTTTATCATAATTCAGCATTC
CACCACTCACGATCTGATCTCAGTGAGAAAAATCAACCTGCAACTCTTGGAACAAGATAAGACAGTCA
TCCATTAGTAATTTTTAAGAAAAAAACGATAGGACCGAAACTAGTATTGAAAGAACCGTCTCGGTCAA
TCTAGGTAATCGAGCTGcTACCGTCTCGGAAAGCTCAAATCATGCTGCCTGATCCGGAAGATCCGGAA
AGCAAAAAAGCTACAAGGAGAACAGGAAACCTAATTATCTGCTTCCTATTCATCTTCTTTCCGTTTGTA
AACTTCATTGTTCCAACTCTAAGACACTTGCTGTCCTAACACCTGCTATAGGCTATCCACTGCATCATC
TCTTTTTAAGAAAAAAATAGGCCCGGACGGGTTAGCAACAAGCGACTGCCGGTGCCAACAACACAAT
CCACAATCTACAGTCGACGCGGCCGCCACCatgttcgtcttcctggtcctgctgcctctggtctcctcac
agtgcgtcaatctgATCactcggactcagTCCtatactaatagcttcaccagaggcgtgtactatcctg
acaaggtgtttagaagctccgtgctgcactctacacaggatctgtttctgccattctttagcaacgtg
acctggttccacgccatcagcggcaccaatggcacaaagcggttcgacaatcccgtgctgccttttaacg
atggcgtgtacttcgcctctaccgagaagagcaacatcatcagaggctggatctttggcaccacactg
gactccaagacacagtctctgctgatcgtgaacaatgccaccaacgtggtcatcaaggtgtgcgagttc
cagttttgtaatgatcccttcctgGACgtgtactatcacaagaacaataagagctggatggagtccgagt
ttagagtgtattctagcgccaacaactgcacatttgagtacgtgagccagcctttcctgatggacctg
gagggcaagcagggcaatttcaagaacctgagggagttcgtgtttaagaatatcgacggctacttcaaa
atctactctaagcacacccccatcaacctgGGCcgcgacctgcctcagggcttcagcgccctggagccc
ctggtggatctgcctatcggcatcaacatcacccggtttcagacactgctggccctgcacagaagctacc
tgacacccggcgactcctctagcggatggaccgccggcgctgccgcctactatgtgggctacctccag
ccccggaccttcctgctgaagtacaacgagaatggcaccatcacagacgcagtggattgcgccctggac
cccctgagcgagacaaagtgtacactgaagtcctttaccgtggagaagggcatctatcagacatcca
atttcagggtgcagccaaccgagtctatcgtgcgctttcctaatatcacaaacctgtgcccatttGAC
gaggtgttcaacgcaacccgcttcgccagcgtgtacgcctggaataggaagcggatcagcaactgcgtgg
ccgactatagcgtgctgtacaacCTGgccCCCttcTTCGCCtttaagtgctatggcgtgtcccccaca
aagctgaatgacctgtgctttaccaacgtctacgccgattctttcgtgatcaggggcAACgaggtgTCC
cagatcgcccccggccagacaggcAATatcgcagactacaattataagctgccagacgatttcaccggc
tgcgtgatcgcctggaacagcaacAAGctggattccaaagtgggcggcaactacaattatAGAtaccggc
tgtttagaaagagcaatctgaagcccttcgagagggacatctctacagaaatctaccaggccggcAAT
AAGccttgcaatggcgtgGCCggcGTGaactgttatttcccactcCAGtcctacggcttcCGCcccacaT
ATggcgtgggcCACcagccttaccgcgtggtggtgctgagctttgagctgctgcacgccccagcaacag
tgtgcggccccaagaagtccaccaatctggtgaagaacaagtgcgtgaacttcaacttcaacggcctg
accggcacaggcgtgctgaccgagtccaacaagaagttcctgccatttcagcagttcggcagggacat
cgcagataccacagacgccgtgcgcgacccacagaccctggagatcctggacatcacaccctgctctttc
ggcggcgtgagcgtgatcacacccggcaccaatacaagcaaccaggtggccgtgctgtatcagGGCgt
gaattgtaccgaggtgcccgtggctatccacgccgatcagctgaccccaacatggcgggtgtacagca
ccggctccaacgtcttccagacaagagccggatgcctgatcggagcagagTACgtgaacaattcctatg
agtgcgacatcccaatcggcgccggcatctgtgcctcttaccagacccagacaAAGtctCACagaagag
cccggagcgtggcctcccagtctatcatcgcctataccatgtccctgggcgccgagaacagcgtggccta
ctctaacaatagcatcgccatcccaaccaacttcacaatctctgtgaccacagagatcctgcccgtgtc
catgaccaagacatctgtggactgcacaatgtatatctgtggcgattctaccgagtgcagcaacctgct
gctccagtacggcagcttttgtacccagctgAAGagagccctgacaggcatcgccgtggagcaggat
aagaacacacaggaggtgttcgcccaggtgaagcaaatctacaagaccccccctatcaagTACtttg
gcggcttcaatttttcccagatcctgcctgatccatccaagccttctaagcggagctttatcgaggac
ctgctgttcaacaaggtgaccctggccgatgccggcttcatcaagcagtatggcgattgcctgggcga
catcgcagccagggacctgatctgcgcccagaagtttaatggcctgaccgtgctgccacccctgctg
acagatgagatgatcgcacagtacacaagcgccctgctggccggcaccatcacatccggatggaccttc
ggcgcaggagccgccctccagatcccctttgccatgcagatggcctataggttcaacggcatcggcgtga
cccagaatgtgctgtacgagaaccagaagctgatcgccaatcagtttaactccgccatcggcaagatcc
aggacagcctgtcctctacagccagcgccctgggcaagctccaggatgtggtgaatCACaacgcccagg
ccctgaataccctggtgaagcagctgagcagcAAGttcggcgccatctctagcgtgctgaatgaca
tcctgagccggctggacaaggtggaggcagaggtgcagatcgaccggctgatcaccggccggctccag
agcctccagacctatgtgacacagcagctgatcagggccgccgagatcagggccagcgccaatctgg
cagcaaccaagatgtccgagtgcgtgctgggccagtctaagagagtggacttttgtggcaagggcta
tcacctgatgtccttccctcagtctgccccacacggcgtggtgtttctgcacgtgacctacgtgcccg
cccaggagaagaacttcaccacagcccctgccatctgccacgatggcaaggcccactttccaagggagg
gcgtgttcgtgtccaacggcacccactggtttgtgacacagcgcaatttctacgagccccagatcatcac
cacagacaacaccttcgtgagcggcaactgtgacgtggtcatcggcatcgtgaacaataccgtgtatgat
ccactccagcccgagctggacagctttaaggaggagctggataagtatttcaagaatcacacctccc
ctgacgtggatctgggcgacatcagcggcatcaatgcctccgtggtgaacatccagaaggagatcgac
cgcctgaacgaggtggctaagaatctgaacgagagcctgatcgacctccaggagctgggcaagtatga
gcagtacatcaagtggccctggtacatctggctgggcttcatcgccggcctgatcgccatcgtgatggt
gaccatcatgctgtgctgtatgacatcctgctgttcttgcctgaagggctgctgtagctgtggctcctg
ctgtAAGTTATTGACCATTGTCGTTGCTAATCGAAATAGAATGGAGAATTTTGTTTATCATAAATGAGCT
AGCCTCCTGCCATACTTCCTACTCACATCATATCTATTTTAAAGAAAAAATAGGCCCGAACACTAATCG
TGCCGGCAGTGCCACTGCACACACAACACTACACATACAATACACTACAATGGTTGCAGAAGATGCCC
CTGTTAGGGGCACTTGCCGAGTATTATTTCGAACAACAACTTTAATTTTTCTATGCACACTATTAGCAT
TAAGCATCTCTATCCTTTATGAGAGTTTAATAACCCAAAAGCAAATCATGAGCCAAGCAGGCTCAACT
GGATCTAATTCTAGATTAGGAAGTATCACTGATCTTCTTAATAATATTCTCTCTGTCGCAAATCAGATT
ATATATAACTCTGCAGTCGCTCTACCTCTACAATTGGACACTCTTGAATCAACACTCCTTACAGCCATT
AAGTCTCTTCAAACAAGTGACAAGCTAGAACAGAACTGCTCGTGGGGTGCTGCACTGATTAATGATAA
TAGATACATTAATGGCATCAATCAGTTCTATTTCTCAATTGCTGAGGGTCGCAAGCTGACACTTGGCCC
ACTTCTTAATATACCTAGTTTCATTCCAACTGCCACGACACCAGAGGGCTGCACCAGGATCCCATCATT
CTCGCTCACCAAGACACACTGGTGTTATACACACAATGTTATCCTGAATGGATGCCAGGATCATGTAT
CCTCAAATCAATTTGTTTCCATGGGAATCATTGAACCCACTTCTGCCGGGTTTCCATCCTTTCGAACCCT
AAAGACTCTATATCTCAGCGATGGGGTCAATCGTAAGAGCTGCTCTATCAGTACAGTTCCGGGGGGTT
GTATGATGTACTGTTTCGTCTCTACTCAACCAGAGAGGGATGACTACTTTTCTACCGCTCCTCCAGAAC
AACGAATTATTATAATGTACTATAATGATACAATCGTGGAGCGCATAATTAATCCACCCGGGGTACTA
GATGTATGGGCAACATTGAACCCAGGAACAGGAAGCGGGGTATATTATTTAGGTTGGGTACTCTTTCC
AATATATGGCGGCGTGATTAAAGATACGAGTTTATGGAATAATCAAGCAAATAAATACTTTATCCCCC
AGATGGTTGCTGCTCTCTGCTCACAAAACCAGGCAACTCAAGTCCAAAATGCTAAGTCATCATACTAT
AGCAGCTGGTTTGGCAATCGAATGATTCAGTCTGGGATCCTGGCATGTCCTCTTCAACAGGATCTAACC
AATGAGTGTTTAGTTCTGCCCTTTTCTAATGATCAGGTGCTTATGGGTGCTGAAGGGAGATTATACATG
TATGGTGACTCGGTGTATTACTACCAAAGAAGCAATAGTTGGTGGCCTATGACCATGCTGTATAAGGT
AACCATAACATTCACTAATGGTCAGCCATCTGCTATATCAGCTCAGAATGTGCCCACACAGCAGGTCC
CTAGACCTGGGACAGGAAGCTGCTCTGCAACAAATAGATGTCCCGGTTTTTGCTTGAAAGGAGTGTAT
GCTGATGCCTGGTTACTGACCAACCCTTCGTCTACCAGTACATTTGGATCAGAAGCAACCTTCACTGGT
TCTTATCTCAACGCAGCAACTCAGCGTATCAATCCGACGATGTATATCGCGAACAACACACAGATCAT
AAGCTCACAGCAATTTGGATCAAGCGGTCAAGAAGCAGCATATGGCCACACAACTTGTTTTAGGGACA
CAGGCTCTGTTATGGTATACTGTATCTATATTATTGAATTGTCCTCATCTCTCTTAGGACAATTTCAGAT
TGTCCCATTTATCCGTCAGGTGACACTATCCTAAAGGCAGAAGCCTCCAGGTCTGACCCAGCCAATCA
AAGCATTATACCAGACCATGGAATGCATACCAAACATTATTGACACTAATGACACACAAAATTGGTTT
TAAGAAAAACCAAGAGAACAATAGGCCAGAATGGCTGGGTCTCGGGAGATATTACTCCCTGAAGTCC
ATCTCAATTCACCAATTGTAAAGCATAAGCTATACTATTACATTCTACTTGGAAACCTCCCAAATGAGA
TCGACATTGACGATTTAGGTCCATTACATAATCAAAATTGGAATCAAATAGCACATGAAGAGTCTAAC
TTAGCCCAACGCTTGGTAAATGTAAGAAATTTTCTAATTACCCACATCCCTGATCTTAGAAAGGGCCAT
TGGCAAGAGTATGTCAATGTAATACTGTGGCCGCGAATTCTTCCCTTGATCCCGGATTTTAAAATCAAT
GACCAATTGCCTCTACTCAAAAATTGGGACAAGTTAGTTAAAGAATCATGTTCAGTAATCAATGCGGG
TACTTCCCAGTGCATTCAGAATCTCAGCTATGGACTGACAGGTCGTGGGAACCTCTTTACACGATCACG
TGAACTCTCTGGTGACCGCAGGGATATTGATCTTAAGACGGTTGTGGCAGCATGGCATGACTCAGACT
GGAAAAGAATAAGTGATTTTTGGATTATGATCAAATTCCAGATGAGACAATTAATTGTTAGGCAAACA
GATCATAATGATCCTGATTTAATCACGTATATCGAAAATAGAGAAGGCATAATCATCATAACCCCTGA
ACTGGTAGCATTATTTAACACTGAGAATCATACACTAACATACATGACCTTTGAAATTGTACTGATGGT
TTCAGATATGTACGAAGGTCGTCACAACATTTTATCACTATGCACAGTTAGCACTTACCTGAATCCTCT
GAAGAAAAGAATAACATATTTATTGAGCCTTGTAGATAACTTAGCTTTTCAGATAGGTGATGCTGTAT
ATAACATAATTGCTTTGCTAGAATCCTTTGTATATGCACAGTTGCAAATGTCAGATCCCATCCCAGAAC
TCAGAGGACAATTCCATGCATTCGTATGTTCTGAGATTCTTGATGCACTAAGGGGAACTAATAGTTTCA
CCCAGGATGAATCAAGAACTGTGACAACCAATTTGATATCCCCATTCCAAGATCTGACCCCAGATCTT
ACGGCTGAATTGCTCTGTATAATGAGGCTTTGGGGACACCCCATGCTCACCGCCAGTCAAGCTGCGGG
AAAGGTACGCGAGTCCATGTGTGCTGGAAAAGTATTAGACTTTCCCACCATTATGAAAACACTAGCCT
TTTTCCATACTATTCTGATCAATGGATACAGGAGGAAGCATCATGGAGTATGGCCACCCTTAAACTTAC
CGGGTAATGCTTCAAAGGGTCTCACGGAACTTATGAATGACAATACTGAGATAAGCTATGAATTCACA
CTTAAGCATTGGAAGGAAATCTCTCTTATAAAATTCAAGAAATGTTTTGATGCAGACGCAGGTGAGGA
ACTCAGTATATTTATGAAAGATAAAGCAATTAGTGCCCCAAAACAAGATTGGATGAGTGTGTTTAGAA
GAAGCCTAATCAAACAGCGCCATCAGCATCATCAGGTCCCCCTACCAAATCCATTCAATCGACGGCTA
TTGCTAAACTTTCTCGGAGATGACAAATTCGACCCGAATGTGGAGCTACAGTATGTAACATCAGGTGA
GTATCTACATGATGACACGTTTTGTGCATCATATTCACTAAAAGAGAAGGAAATTAAACCTGATGGTC
GAATTTTTGCAAAGTTGACTAAGAGAATGAGATCATGTCAAGTTATAGCAGAATCTCTTTTAGCGAAC
CATGCTGGGAAGTTAATGAAAGAGAATGGTGTTGTGATGAATCAGCTATCATTAACAAAATCACTATT
AACAATGAGTCAGATTGGAATAATATCCGAGAAAGCTAGAAAATCGACTCGAGATAACATAAATCAA
CCTGGTTTCCAGAATATCCAGAGAAATAAATCACATCACTCCAAGCAAGTCAATCAGCGAGATCCAAG
TGATGACTTTGAATTGGCAGCATCTTTTTTAACTACTGATCTCAAAAAATATTGTTTACAATGGAGGTA
CCAGACAATTATCCCATTTGCTCAATCATTAAACAGAATGTATGGTTATCCTCATCTCTTTGAGTGGAT
TCACTTACGGCTAATGCGTAGTACACTTTACGTGGGGGATCCCTTCAACCCACCAGCAGATACCAGTC
AATTTGATCTAGATAAAGTAATTAATGGAGATATCTTCATTGTATCACCCAGAGGTGGAATTGAAGGG
CTATGTCAAAAGGCTTGGACAATGATATCTATCTCTGTGATAATTCTATCTGCCACAGAGTCTGGCACA
CGAGTAATGAGTATGGTGCAGGGAGATAATCAAGCAATTGCTGTCACCACACGAGTACCAAGGAGCC
TGCCGACTCTTGAGAAAAAGACTATTGCTTTTAGATCTTGTAATCTATTCTTTGAGAGGTTAAAATGTA
ATAATTTTGGATTAGGTCACCATTTGAAAGAACAAGAGACTATCATTAGTTCTCACTTCTTTGTTTATA
GCAAGAGAATATTCTATCAGGGGAGGATTCTAACGCAAGCCTTAAAAAATGCTAGTAAGCTCTGCTTG
ACAGCTGATGTCCTAGGAGAATGTACCCAATCATCATGTTCTAATCTTGCAACTACTGTCATGAGGTTA
ACTGAGAATGGTGTTGAAAAAGATATCTGTTTCTACTTGAATATCTATATGACCATCAAACAGCTCTCC
TATGATATCATCTTCCCTCAAGTGTCAATTCCTGGAGATCAGATCACATTAGAATACATAAATAATCCA
CACCTGGTATCACGATTGGCTCTTCTGCCATCCCAGCTAGGAGGTCTAAACTACCTGTCATGCAGTAGG
CTGTTCAATCGAAACATAGGCGACCCGGTGGTTTCCGCAGTTGCAGATCTTAAGAGATTAATTAAATC
AGGATGTATGGATTACTGGATCCTTTATAACTTATTAGGGAGAAAACCGGGAAACGGCTCATGGGCTA
CTTTAGCAGCTGACCCGTACTCAATCAATATAGAGTATCAATACCCCCCAACTACAGCTCTTAAGAGG
CACACCCAACAAGCTCTGATGGAACTCAGTACGAATCCAATGTTACGTGGCATATTCTCTGACAATGC
ACAGGCAGAAGAAAATAATCTTGCTAGATTTCTCCTGGATAGGGAGGTGATCTTTCCGCGTGTAGCTC
ACATCATCATTGAGCAAACCAGTGTCGGGAGGAGAAAACAGATTCAAGGATATTTGGATTCAACTAG
ATCGATAATGAGTAAATCACTAGAAATTAAGCCCTTGTCCAATAGGAAGCTTAATGAAATACTGGATT
ACAACATCAATTACCTAGCTTACAATTTGGCATTACTCAAGAATGCTATTGAACCTCCGACTTATTTGA
AAGCAATGACTCTTGAAACATGTAGCATCGACATTGCAAGGAGCCTCCGGAAGCTCTCCTGGGCCCCA
CTCTTGGGTGGGAGAAATCTTGAAGGATTAGAGACGCCAGATCCCATTGAAATTACTGCAGGAGCATT
AATTGTTGGATCGGGCTACTGTGAACAGTGTGCTGCAGGAGACAATCGATTCACATGGTTTTTCTTGCC
ATCTGGTATCGAGATAGGAGGGGATCCCCGTGATAATCCTCCTATCCGTGTACCGTACATTGGCTCCA
GGACTGATGAGAGGAGGGTAGCCTCAATGGCATACATCAGGGGTGCCTCGAGTAGCCTAAAAGCAGT
TCTTAGACTGGCGGGAGTGTACATCTGGGCATTCGGAGATACTCTGGAGAATTGGATAGATGCACTGG
ATTTGTCTCACACTAGAGTTAACATCACACTTGAACAGCTGCAATCCCTCACCCCACTTCCAACCTCTG
CCAATCTAACCCATCGGTTGGATGATGGCACAACTACCCTAAAGTTTACTCCTGCGAGCTCTTACACCT
TTTCAAGTTTCACTCATATATCAAATGATGAGCAATACCTGACAATTAATGACAAAACTGCAGATTCA
AATATAATCTACCAACAGTTAATGATCACTGGACTCGGAATTTTAGAAACATGGAATAATCCCCCAAT
CAATAGAACATTCGAAGAATCTACCCTACATTTGCACACTGGTGCATCATGTTGTGTCCGACCTGTGGA
CTCCTGCATCATCTCAGAAGCATTAACAGTCAAGCCACATATTACAGTACCGTACAGCAATAAATTTG
TATTTGATGAAGACCCGCTATCTGAATATGAGACTGCAAAACTGGAATCGTTATCATTTCAAGCCCAA
TTAGGCAACATTGATGCTGTAGATATGACAGGTAAATTAACATTATTGTCCCAATTCACTGCAAGGCA
GATTATTAATGCAATCACTGGACTCGATGAGTCTGTCTCTCTTACTAATGATGCCATTGTTGCATCAGA
CTATGTCTCCAATTGGATTAGTGAATGCATGTATACCAAATTAGATGAATTATTTATGTATTGTGGGTG
GGAACTACTATTGGAACTATCCTATCAAATGTATTATCTGAGGGTAGTTGGGTGGAGTAATATAGTGG
ATTATTCTTACATGATCTTGAGAAGAATCCCGGGTGCAGCATTAAACAATCTGGCATCTACATTAAGTC
ATCCAAAACTTTTCCGACGAGCTATCAACCTAGATATAGTTGCCCCCTTAAATGCTCCTCATTTTGCAT
CTCTGGACTACATCAAGATGAGTGTGGATGCAATACTCTGGGGCTGTAAAAGAGTCATCAATGTGCTC
TCCAATGGAGGGGACTTAGAATTAGTTGTGACATCTGAAGATAGCCTTATTCTCAGTGACCGATCCAT
GAATCTCATTGCAAGGAAATTAACTTTATTATCACTGATTCACCATAATGGTTTGGAACTACCAAAGAT
TAAGGGGTTCTCTCCTGATGAGAAGTGTTTCGCTTTGACAGAATTTTTGAGGAAAGTGGTGAACTCAG
GGTTGAGTTCAATAGAGAACCTATCAAATTTTATGTACAATGTGGAGAACCCACGGCTTGCAGCATTC
GCCAGCAACAATTACTACCTGACCAGAAAATTATTGAATTCAATACGAGATACTGAGTCGGGTCAAGT
AGCAGTCACCTCATATTATGAATCATTAGAATATATTGATAGTCTTAAGCTAACCCCACATGTGCCTGG
TACCTCATGCATTGAGGATGATAGTCTATGTACAAATGATTACATAATCTGGATCATAGAGTCTAATGC
AAACTTGGAGAAGTATCCAATTCCAAATAGCCCTGAGGATGATTCCAATTTCCATAACTTTAAGTTGA
ATGCTCCATCGCACCATACCTTACGCCCATTAGGGTTGTCATCAACTGCTTGGTATAAGGGTATAAGCT
GTTGCAGGTACCTTGAGCGATTAAAGCTACCACAAGGTGATCATTTATATATTGCAGAAGGTAGTGGT
GCCAGTATGACAATCATAGAATACCTATTCCCAGGAAGAAAGATATATTACAATTCTTTATTTAGTAGT
GGTGACAATCCCCCACAAAGAAATTATGCACCAATGCCTACTCAGTTCATTGAGAGTGTCCCATACAA
GCTCTGGCAAGCACACACAGATCAATATCCCGAGATTTTTGAGGACTTCATCCCTCTATGGAACGGAA
ATGCCGCCATGACTGACATAGGAATGACAGCTTGTGTAGAATTCATCATCAATCGAGTCGGCCCAAGG
ACTTGCAGTTTAGTACATGTAGATTTGGAATCAAGTGCAAGCTTAAATCAACAATGCCTGTCAAAGCC
GATAATTAATGCTATCATCACTGCTACAACTGTTTTGTGCCCTCATGGGGTGCTTATTCTGAAATATAG
TTGGTTGCCATTTACTAGATTTAGTACTTTGATCACTTTCTTATGGTGCTACTTTGAGAGAATCACTGTT
CTTAGGAGCACATATTCTGGTCCAGCTAATCATGAGGTTTATTTAATTTGTATCCTTGCCAACAACTTT
GCATTCCAGACTGTCTCGCAGGCAACAGGAATGGCGATGACTTTAACCGATCAAGGGTTTACTTTGAT
ATCACCTGAAAGAATAAATCAGTATTGGGATGGTCACTTGAAGCAAGAACGTATCGTAGCAGAAGCA
ATTGATAAGGTGGTTCTAGGAGAAGATGCTCTATTCAATTCGAGTGATAATGAATTAATTCTCAAATGT
GGAGGGACACCAAATGCACGGAATCTTATCGATATCGAGCCAGTCGCAACTTTCATAGAATTTGAACA
ACTGATCTGCACAATGTTGACAACCCACTTGAAGGAAATAATTGATATAACAAGGTCTGGAACCCAGG
ATTATGAAAGTTTATTACTCACTCCTTACAATTTAGGTCTTCTTGGTAAAATCAGTACGATAGTGAGAT
TATTAACAGAAAGGATTCTAAATCATACTATCAGGAATTGGTTGATCCTCCCACCTTCGCTCCGGATGA
TCGTGAAGCAGGACTTGGAATTCGGCATATTCAGGATTACTTCCATCCTCAATTCTGATCGGTTCCTGA
AGCTTTCTCCAAATAGGAAATACTTGATTACACAATTAACTGCAGGCTACATTAGGAAATTGATTGAG
GGGGATTGTAATATCGATCTAACCAGACCTATCCAAAAACAAATCTGGAAAGCATTAGGTTGTGTAGT
CTATTGTCACGATCCAGTAGATCAAAGGGAATCAACAGAGTTTATTGATATAAATATTAATGAAGAAA
TAGACCGCGGGATCGATGGCGAGGAAATCTAAATATATCAAGAATCAGAATTAGTTTAAGAAAAAAG
AAGAGGATTAATCTTGGTTTTCCCCTTGGT.

20. CVL111

The nucleic acid sequence for CVL111 is:

(SEQ ID NO: 79)
ACCAGGGGGAAAACGAAGTGGTGACTCAAATCATCGAAGACCCTCGAGATTACATAGGTCCGGAACC
TATGGCCTTCGTGACCGACCTCGAGTCAGAGTAGTTCAATAAGGACCTATCAAGTTTGGGCAATTTTTC
GTCCCCGACACAAAAATGTCATCCGTGCTTAAAGCATATGAGCGATTCACACTCACTCAAGAACTGCA
AGATCAGAGTGAGGAAGGTACAATCCCACCTACAACACTAAAACCGGTAATCAGGGTATTTATACTAA
CCTCTAATAACCCAGAGCTAAGATCCCGGCTTCTTCTATTCTGCCTACGGATTGTTCTCAGTAATGGTG
CAAGGGATTCCCATCGCTTTGGAGCATTACTTACAATGTTTTCGCTACCATCAGCCACAATGCTCAATC
ATGTCAAATTAGCTGACCAGTCACCAGAAGCTGATATCGAAAGGGTAGAGATCGATGGCTTTGAGGA
GGGATCATTCCGCTTAATCCCCAATGCTCGTTCAGGTATGAGCCGTGGAGAGATCAATGCCTATGCTG
CACTTGCAGAAGATCTACCTGACACACTAAACCATGCAACACCTTTTGTTGATTCCGAAGTCGAGGGA
ACTGCATGGGATGAGATTGAGACTTTCTTAGATATGTGTTACAGTGTCCTAATGCAGGCATGGATAGT
GACTTGCAAGTGCATGACTGCGCCAGACCAACCTGCTGCTTCTATTGAGAAACGCCTGCAAAAATATC
GTCAGCAAGGCAGGATCAACCCGAGATATCTCCTGCAACCGGAGGCTCGAAGAATAATCCAGAATGT
AATCCGAAAGGGAATGGTGGTCAGACATTTCCTCACCTTTGAACTGCAGCTTGCCCGAGCACAAAGCC
TTGTATCAAATAGGTATTATGCTATGGTAGGGGATGTTGGAAAGTATATAGAGAATTGTGGAATGGGA
GGCTTCTTTTTGACACTAAAATATGCATTAGGAACCAGATGGCCCACACTTGCTTTAGCTGCATTTTCA
GGAGAGCTAACAAAGCTAAAGTCCCTCATGGCATTATACCAGACCCTTGGTGAGCAGGCCCGATATTT
GGCCCTATTGGAGTCACCACATTTGATGGATTTTGCTGCAGCAAACTACCCACTGCTATATAGCTATGC
TATGGGAATAGGCTATGTGTTAGATGTCAACATGAGGAACTACGCTTTCTCCAGATCATACATGAATA
AGACATATTTCCAATTGGGAATGGAAACTGCAAGAAAACAACAGGGTGCAGTTGACATGAGGATGGC
AGAAGATCTCGGTCTAACTCAAGCCGAACGCACCGAGATGGCAAATACACTTGCCAAATTGACCACA
GCAAATCGAGGGGCAGACACCAGGGGAGGAGTCAACCCGTTCTCATCTATCACTGGGACAACTCAGG
TGCCCGCTGCAGCAACAGGTGACACATTCGAGAGTTACATGGCAGCGGATCGACTGAGGCAGAGATA
TGCTGATGCAGGCACCCACGATGATGAGATGCCACCATTGGAAGAGGAGGAAGAGGACGACACATCT
GCAGGTCCACGCACTGGACTAACTCTTGAACAAGTGGCCTTGGACATCCAGAACGCAGCAGTTGGAGC
TCCCATCCATACAGATGACCTGAATGCCGCACTGGGTGATCTTGACATCTAGACAATTCAGATCCCAA
TCTTAAATCGACACACCTAATTGACCAGTTAGATGGAACTACAGTGGATTCCATAAGGTTCCTGCCTAC
CATCGGCTTTTAAGAAAAAAATAGGCCCGGACGGGTTAGCAACAAGCGACTGCCGATGCCAATAACA
CAATCCACAATCTACAATGGATCCCACTGATCTGAGCTTCTCCCCAGATGAGATCAATAAGCTCATAG
AGACAGGCCTGAATACTGTGGAGTATTTTACTTCCCAACAAGTCACAGGAACATCCTCTCTTGGAAAG
AATACAATACCACCAGGGGTCACAGGACTACTAACCAATGCTGCAGAGGCAAAGATCCAAGAGTCAA
CCAACCATCAGAAGGGTTCAGTTGGTGGGGGCGCAAAACCAAAGAAACCGCGACCAAAAATTGCCAT
TGTGCCAGCAGATGACAAAACGGTGCCCGGAAAGCCGATCCCAAACCCTCTATTAGGTCTGGACTCCA
CCCCGAGCACCCAAACTGTGCTTGATCTAAGTGGGAAAACATTACCATCAGGATCCTATAAGGGGGTT
AAACTTGCGAAATTTGGAAAAGAAAATCTGATGACACGGTTCATCGAGGAACCCAGAGAGAATCCTA
TCGCAACCAATTCCCCCATCGATTTTAAGAGGGGCAGGGATACCGGCGGGTTCCATAGAAGGGAGTAC
TCAATCGGATGGGTGGGAGATGAAGTCAAGGTCACTGAGTGGTGCAATCCATCCTGTTCTCCAATCAC
CGCTGCAGCAAGGCGATTTGAATGCACTTGTCACCAGTGTCCAGTCACTTGCTCTGAATGTGAACGAG
ATACTTAATACAGTGAGAAATTTGGACTCTCGGATGAATCAACTGGAGACAAAAGTAGATCGCATTCT
CTCATCTCAGTCTCTAATCCAGACCATCAAGAATGACATAGTTGGACTTAAAGCAGGGATGGCTACTT
TAGAAGGAATGATTACAACTGTGAAAATCATGGACCCGGGAGTTCCCAGTAATGTTACTGTGGAAGAT
GTACGCAAGAAACTAAGTAACCATGCTGTTGTTGTGCCAGAATCATTCAATGATAGTTTCTTGACTCAA
TCTGAAGATGTAATTTCACTTGATGAGTTGGCTCGACCAACTGCAACAAGTGTTAAGAAGATTGTCAG
GAAGGTTCCTCCTCAGAAGGATCTGACTGGATTGAAGATCACACTAGAGCAATTGGCAAAGGATTGCA
TCAGCAAACCGAAGATGAGGGAAGAGTATCTCCTCAAAATCAACCAGGCTTCCAGTGAGGCTCAGCT
AATTGACCTCAAGAAAGCAATCATCCGCAGTGCAATTTGATCAAGAAACACCCAATTACACTACACTG
GTATGACACTGTACTAACCCTGAGGGTTTTAGAAAAAACGATTAACGATAAATAAGCCCGAACACTAC
ACACCACCCGAGGCAGCCATGCCATCCATCAGCATCCCCGCAGACCCCACCAATCCACGTCAATCAAT
AAAAGCGTTCCCAATTGTGATCAACCGTGATGGGGGTGAGAAAGGTCGCTTGGTTAAACAACTACGCA
CAACCTACTTGAATGACCTAGATACTCATGAGCCACTGGTGACATTCGTAAATACTTATGGATTCATCT
ACGAACAGGATCGGGGAAATACCATTGTCGGAGAGGATCAACATGGGAAGAAAAGAGAGGCTGTGA
CTGCTGCAATGGTTACCCTTGGATGTGGGCCTAATCTACCATCATTAGGGAATGTCCTGGGACAACTG
AGTGAATTCCAGGTCATTGTTAGGAAGACATCCAGCAAAGCGGAAGAGATGGTCTTTGAAATTGTTAA
GTATCCGAGAATATTTCGGGGTCATACATTAATCCAGAAAGGACTAGTCTGTGTCTCCGCAGAAAAAT
TTGTTAAGTCACCAGGGAAAGTACAATCTGGAATGGACTATCTCTTCATTCCGACATTTCTGTCAGTGA
CTTACTGTCCAGCTGCAATCAAATTTCAGGTACCTGGCCCCATGTTGAAAATGAGATCAAGATACACT
CAGAGCTTACAACTTGAACTAATGATAAGAATCCTGTGTAAGCCCGATTCGCCACTTATGAAGGTCCA
TATCCCTGACAAGGAAGGAAGAGGATGTCTTGTATCAGTATGGTTGCATGTATGCAATATCTTCAAAT
CAGGAAACAAGAATGGCAGTGAGTGGCAGGAATACTGGATGAGAAAGTGTGCTAACATGCAACTTGA
AGTGTCGATTGCAGATATGTGGGGACCAACTATCATAATTCATGCCAGAGGTCACATTCCCAAAAGTG
CTAAGTTGTTTTTTGGAAAGGGTGGATGGAGCTGCCATCCACTTCACGAAGTTGTTCCAAGTGTCACTA
AAACACTATGGTCCGTGGGCTGTGAGATTACAAAGGCGAAGGCAATAATACAAGAGAGTAGCATCTC
TCTTCTCGTGGAGACTACTGACATCATAAGTCCAAAAGTCAAAATTTCATCTAAGCATCGCCGCTTTGG
GAAATCAAATTGGGGTCTGTTCAAGAAAACCAAATCACTGCCCAACCTGACGGAGCTGGAATGACTG
ACCCCCAATCGAGACTACACCACCTCAAACTATAGGTGGGTGGTACCTCAGTGATTAATCTCGTAAGC
ACTGATCGTAGGCTACAACACACTAATATTATCCAGATTAGAGAGCTTAATTAGCTCTGTATTAATAAT
AACACTACTATTCCAATAACTGGAATCACCAGCTTGATTTATCTCCAAAATGATTCAAAGAAAACAAA
TCATATTAAGACTATCCTAAGCACGAACCCATATCGTCCTTCAAATCATGGGTACTATAATTCAATTTC
TGGTGGTCTCCTGTCTATTGGCAGGAGCAGGCAGTCTTGATCCAGCAGCCCTCATGCAAATCGGTGTC
ATTCCAACAAATGTCCGGCAACTTATGTATTATACTGAGGCCTCATCAGCATTCATTGTTGTGAAGTTA
ATGCCTACAATTGACTCGCCGATTAGTGGATGTAATATAACATCAATTTCAAGCTATAATGCAACAGT
GACAAAACTCCTACAGCCGATCGGTGAGAATTTGGAGACGATTAGGAACCAGTTGATTCCAACTCGGA
GGAGACGCCGGTTTGCAGGGGTGGTGATTGGATTAGCTGCATTAGGAGTAGCTACTGCCGCACAGGTC
ACTGCCGCAGTAGCACTAGTAAAGGCAAATGAAAATGCTGCGGCTATACTCAATCTCAAAAATGCAAT
CCAAAAAACAAATGCAGCAGTTGCAGATGTGGTCCAGGCCACACAATCACTAGGAACaGCAGTTCAAG
CAGTTCAAGATCACATAAACAGTGTGGTAAGTCCAGCAATTACAGCAGCCAATTGTAAAGCCCAAGAT
GCTATCATTGGCTCAATCCTCAATCTCTATTTGACCGAGTTGACAACTATCTTCCACAATCAAATTACA
AACCCTGCATTGAGTCCTATTACAATTCAAGCTTTAAGGATCCTACTAGGGAGTACCTTGCCGACTGTG
GTCGAAAAATCTTTCAATACCCAGATAAGTGCGGCTGAGCTTCTCTCATCAGGGTTATTGACAGGCCA
GATTGTGGGATTAGATTTGACCTATATGCAGATGGTCATAAAAATTGAGCTGCCAACTTTAACTGTAC
AACCTGCAACCCAGATCATAGATCTGGCCACCATTTCTGCATTCATTAACAATCAAGAAGTCATGGCC
CAATTACCAACACGTGTTATTGTGACTGGCAGCTTGATCCAAGCCTATCCCGCATCGCAATGCACTATT
ACACCCAACACTGTGTACTGTAGGTATAATGATGCCCAAGTACTCTCAGATGATACGATGGCTTGCCT
CCAAGGTAACTTGACAAGATGCACCTTCTCTCCAGTGGTTGGGAGCTTTCTCACTCGATTCGTGCTGTT
CGATGGAATAGTTTATGCAAATTGCAGGTCGATGTTGTGCAAGTGCATGCAGCCTGCTGCTGTGATCCT
ACAGCCGAGTTCATCCCCTGTAACTGTCATTGACATGTACAAATGTGTGAGTCTGCAGCTTGACAATCT
CAGATTCACCATCACTCAATTGGCCAATGTAACCTACAATAGCACCATCAAGCTTGAAACATCCCAGA
TCTTGCCTATTGATCCGTTGGATATATCCCAGAATCTAGCTGCGGTGAATAAGAGTCTAAGTGATGCAC
TACAACACTTAGCACAAAGTGACACATACCTTTCTGCAATCACATCAGCTACGACTACAAGTGTATTA
TCCATAATAGCAATCTGTCTTGGATCGTTAGGTTTAATATTAATAATCTTGCTCAGTGTAGTTGTGTGG
AAGTTATTGACCATTGTCGCTGCTAATCGAAATAGAATGGAGAATTTTGTTTATCATAATTCAGCATTC
CACCACTCACGATCTGATCTCAGTGAGAAAAATCAACCTGCAACTCTTGGAACAAGATAAGACAGTCA
TCCATTAGTAATTTTTAAGAAAAAAACGATAGGACCGAAACTAGTATTGAAAGAACCGTCTCGGTCAA
TCTAGGTAATCGAGCTGcTACCGTCTCGGAAAGCTCAAATCATGCTGCCTGATCCGGAAGATCCGGAA
AGCAAAAAAGCTACAAGGAGAACAGGAAACCTAATTATCTGCTTCCTATTCATCTTCTTTCCGTTTGTA
AACTTCATTGTTCCAACTCTAAGACACTTGCTGTCCTAACACCTGCTATAGGCTATCCACTGCATCATC
TCTTTTTAAGAAAAAAATAGGCCCGGACGGGTTAGCAACAAGCGACTGCCGGTGCCAACAACACAAT
CCACAATCTACAGTCGACGCGGCCGCCACCatgttcgtcttcctggtcctgctgcctctggtctcctc
acagtgcgtcaatctgATCactcggactcagTCCtatactaatagcttcaccagaggcgtgtactatcc
tgacaaggtgtttagaagctccgtgctgcactctacacaggatctgtttctgccattctttagcaacgt
gacctggttccacgccatccacgtgagcggcaccaatggcacaaagcggttcgacaatcccgtgctg
ccttttaacgatggcgtgtacttcgcctctaccgagaagagcaacatcatcagaggctggatctttggc
accacactggactccaagacacagtctctgctgatcgtgaacaatgccaccaacgtggtcatcaaggtgt
gcgagttccagttttgtaatgatcccttcctgGACgtgtactatcacGAGaacaataagagcAGAatgga
gtccgagCTGagagtgtattctagcgccaacaactgcacatttgagtacgtgagccagcctttcctga
tggacctggagggcaagcagggcaatttcaagaacctgagggagttcgtgtttaagaatatcgacggc
tacttcaaaatctactctaagcacacccccGTGaacctgGGCcgcgacctgcctcagggcttcag
cgccctggagcccctggtggatctgcctatcggcatcaacatcacccggtttcagacactgctggcc
ctgcacagaagctacctgacacccggcgactcctctagcAGCtggaccgccggcgctgccgcctacta
tgtgggctacctccagccccggaccttcctgctgaagtacaacgagaatggcaccatcacagacgcag
tggattgcgccctggaccccctgagcgagacaaagtgtacactgaagtcctttaccgtggagaagggc
atctatcagacatccaatttcagggtgcagccaaccgagtctatcgtgcgctttcctaatatcacaaacc
tgtgcccatttCACgaggtgttcaacgcaacccgcttcgccagcgtgtacgcctggaataggaagcgga
tcagcaactgcgtggccgactatagcgtgctgtacaacCTGgccCCCttcTTCGCCtttaagtgctatg
gcgtgtcccccacaaagctgaatgacctgtgctttaccaacgtctacgccgattctttcgtgatcagg
ggcAACgaggtgTCCcagatcgcccccggccagacaggcAATatcgcagactacaattataagctgccag
acgatttcaccggctgcgtgatcgcctggaacagcaacAAGctggattccaaagtgTCCggcaactac
aattatctgtaccggctgtttagaaagagcAAGctgaagcccttcgagagggacatctctacagaaat
ctaccaggccggcAATAAGccttgcaatggcgtgGCCggctttaactgttatttcccactcCAGtccta
cggcttcCGCcccacaTATggcgtgggcCACcagccttaccgcgtggtggtgctgagctttgagctgctg
cacgccccagcaacagtgtgcggccccaagaagtccaccaatctggtgaagaacaagtgcgtgaacttca
acttcaacggcctgaccggcacaggcgtgctgaccgagtccaacaagaagttcctgccatttcagca
gttcggcagggacatcgcagataccacagacgccgtgcgcgacccacagaccctggagatcctggaca
tcacaccctgctctttcggcggcgtgagcgtgatcacacccggcaccaatacaagcaaccaggtggcc
gtgctgtatcagGGCgtgaattgtaccgaggtgcccgtggctatccacgccgatcagctgaccccaa
catggcgggtgtacagcaccggctccaacgtcttccagacaagagccggatgcctgatcggagcagagTA
Cgtgaacaattcctatgagtgcgacatcccaatcggcgccggcatctgtgcctcttaccagacccagac
aAAGtctCACagaagagcccggagcgtggcctcccagtctatcatcgcctataccatgtccctgggcgcc
gagaacagcgtggcctactctaacaatagcatcgccatcccaaccaacttcacaatctctgtgaccacag
agatcctgcccgtgtccatgaccaagacatctgtggactgcacaatgtatatctgtggcgattcta
ccgagtgcagcaacctgctgctccagtacggcagcttttgtacccagctgAAGagagccctgacaggc
atcgccgtggagcaggataagaacacacaggaggtgttcgcccaggtgaagcaaatctacaagaccccc
cctatcaagTACtttggcggcttcaatttttcccagatcctgcctgatccatccaagccttctaagcgga
gctttatcgaggacctgctgttcaacaaggtgaccctggccgatgccggcttcatcaagcagtatggcga
ttgcctgggcgacatcgcagccagggacctgatctgcgcccagaagtttaatggcctgaccgtgctgcc
acccctgctgacagatgagatgatcgcacagtacacaagcgccctgctggccggcaccatcacatc
cggatggaccttcggcgcaggagccgccctccagatcccctttgccatgcagatggcctataggttca
acggcatcggcgtgacccagaatgtgctgtacgagaaccagaagctgatcgccaatcagtttaactcc
gccatcggcaagatccaggacagcctgtcctctacagccagcgccctgggcaagctccaggatgtgg
tgaatCACaacgcccaggccctgaataccctggtgaagcagctgagcagcAAGttcggcgccatctcta
gcgtgctgaatgacatcctgagccggctggacaaggtggaggcagaggtgcagatcgaccggctgatca
ccggccggctccagagcctccagacctatgtgacacagcagctgatcagggccgccgagatcagggccag
cgccaatctggcagcaaccaagatgtccgagtgcgtgctgggccagtctaagagagtggacttttgtg
gcaagggctatcacctgatgtccttccctcagtctgccccacacggcgtggtgtttctgcacgtgacct
acgtgcccgcccaggagaagaacttcaccacagcccctgccatctgccacgatggcaaggcccactttc
caagggagggcgtgttcgtgtccaacggcacccactggtttgtgacacagcgcaatttctacgagcccca
gatcatcaccacagacaacaccttcgtgagcggcaactgtgacgtggtcatcggcatcgtgaacaat
accgtgtatgatccactccagcccgagctggacagctttaaggaggagctggataagtatttcaagaatc
acacctcccctgacgtggatctgggcgacatcagcggcatcaatgcctccgtggtgaacatccagaagg
agatcgaccgcctgaacgaggtggctaagaatctgaacgagagcctgatcgacctccaggagctgggca
agtatgagcagtacatcaagtggccctggtacatctggctgggcttcatcgccggcctgatcgccatc
gtgatggtgaccatcatgctgtgctgtatgacatcctgctgttcttgcctgaagggctgctgtagct
gtggctcctgctgtAAGTTATTGACCATTGTCGTTGCTAATCGAAATAGAATGGAGAATTTTGTTTATCA
TAAATGAGCTAGCCTCCTGCCATACTTCCTACTCACATCATATCTATTTTAAAGAAAAAATAGGCCCGA
ACACTAATCGTGCCGGCAGTGCCACTGCACACACAACACTACACATACAATACACTACAATGGTTGCA
GAAGATGCCCCTGTTAGGGGCACTTGCCGAGTATTATTTCGAACAACAACTTTAATTTTTCTATGCACA
CTATTAGCATTAAGCATCTCTATCCTTTATGAGAGTTTAATAACCCAAAAGCAAATCATGAGCCAAGC
AGGCTCAACTGGATCTAATTCTAGATTAGGAAGTATCACTGATCTTCTTAATAATATTCTCTCTGTCGC
AAATCAGATTATATATAACTCTGCAGTCGCTCTACCTCTACAATTGGACACTCTTGAATCAACACTCCT
TACAGCCATTAAGTCTCTTCAAACAAGTGACAAGCTAGAACAGAACTGCTCGTGGGGTGCTGCACTGA
TTAATGATAATAGATACATTAATGGCATCAATCAGTTCTATTTCTCAATTGCTGAGGGTCGCAAGCTGA
CACTTGGCCCACTTCTTAATATACCTAGTTTCATTCCAACTGCCACGACACCAGAGGGCTGCACCAGGA
TCCCATCATTCTCGCTCACCAAGACACACTGGTGTTATACACACAATGTTATCCTGAATGGATGCCAGG
ATCATGTATCCTCAAATCAATTTGTTTCCATGGGAATCATTGAACCCACTTCTGCCGGGTTTCCATCCTT
TCGAACCCTAAAGACTCTATATCTCAGCGATGGGGTCAATCGTAAGAGCTGCTCTATCAGTACAGTTC
CGGGGGGTTGTATGATGTACTGTTTCGTCTCTACTCAACCAGAGAGGGATGACTACTTTTCTACCGCTC
CTCCAGAACAACGAATTATTATAATGTACTATAATGATACAATCGTGGAGCGCATAATTAATCCACCC
GGGGTACTAGATGTATGGGCAACATTGAACCCAGGAACAGGAAGCGGGGTATATTATTTAGGTTGGGT
ACTCTTTCCAATATATGGCGGCGTGATTAAAGATACGAGTTTATGGAATAATCAAGCAAATAAATACT
TTATCCCCCAGATGGTTGCTGCTCTCTGCTCACAAAACCAGGCAACTCAAGTCCAAAATGCTAAGTCAT
CATACTATAGCAGCTGGTTTGGCAATCGAATGATTCAGTCTGGGATCCTGGCATGTCCTCTTCAACAGG
ATCTAACCAATGAGTGTTTAGTTCTGCCCTTTTCTAATGATCAGGTGCTTATGGGTGCTGAAGGGAGAT
TATACATGTATGGTGACTCGGTGTATTACTACCAAAGAAGCAATAGTTGGTGGCCTATGACCATGCTG
TATAAGGTAACCATAACATTCACTAATGGTCAGCCATCTGCTATATCAGCTCAGAATGTGCCCACACA
GCAGGTCCCTAGACCTGGGACAGGAAGCTGCTCTGCAACAAATAGATGTCCCGGTTTTTGCTTGAAAG
GAGTGTATGCTGATGCCTGGTTACTGACCAACCCTTCGTCTACCAGTACATTTGGATCAGAAGCAACCT
TCACTGGTTCTTATCTCAACGCAGCAACTCAGCGTATCAATCCGACGATGTATATCGCGAACAACACA
CAGATCATAAGCTCACAGCAATTTGGATCAAGCGGTCAAGAAGCAGCATATGGCCACACAACTTGTTT
TAGGGACACAGGCTCTGTTATGGTATACTGTATCTATATTATTGAATTGTCCTCATCTCTCTTAGGACA
ATTTCAGATTGTCCCATTTATCCGTCAGGTGACACTATCCTAAAGGCAGAAGCCTCCAGGTCTGACCCA
GCCAATCAAAGCATTATACCAGACCATGGAATGCATACCAAACATTATTGACACTAATGACACACAAA
ATTGGTTTTAAGAAAAACCAAGAGAACAATAGGCCAGAATGGCTGGGTCTCGGGAGATATTACTCCCT
GAAGTCCATCTCAATTCACCAATTGTAAAGCATAAGCTATACTATTACATTCTACTTGGAAACCTCCCA
AATGAGATCGACATTGACGATTTAGGTCCATTACATAATCAAAATTGGAATCAAATAGCACATGAAGA
GTCTAACTTAGCCCAACGCTTGGTAAATGTAAGAAATTTTCTAATTACCCACATCCCTGATCTTAGAAA
GGGCCATTGGCAAGAGTATGTCAATGTAATACTGTGGCCGCGAATTCTTCCCTTGATCCCGGATTTTAA
AATCAATGACCAATTGCCTCTACTCAAAAATTGGGACAAGTTAGTTAAAGAATCATGTTCAGTAATCA
ATGCGGGTACTTCCCAGTGCATTCAGAATCTCAGCTATGGACTGACAGGTCGTGGGAACCTCTTTACA
CGATCACGTGAACTCTCTGGTGACCGCAGGGATATTGATCTTAAGACGGTTGTGGCAGCATGGCATGA
CTCAGACTGGAAAAGAATAAGTGATTTTTGGATTATGATCAAATTCCAGATGAGACAATTAATTGTTA
GGCAAACAGATCATAATGATCCTGATTTAATCACGTATATCGAAAATAGAGAAGGCATAATCATCATA
ACCCCTGAACTGGTAGCATTATTTAACACTGAGAATCATACACTAACATACATGACCTTTGAAATTGTA
CTGATGGTTTCAGATATGTACGAAGGTCGTCACAACATTTTATCACTATGCACAGTTAGCACTTACCTG
AATCCTCTGAAGAAAAGAATAACATATTTATTGAGCCTTGTAGATAACTTAGCTTTTCAGATAGGTGAT
GCTGTATATAACATAATTGCTTTGCTAGAATCCTTTGTATATGCACAGTTGCAAATGTCAGATCCCATC
CCAGAACTCAGAGGACAATTCCATGCATTCGTATGTTCTGAGATTCTTGATGCACTAAGGGGAACTAA
TAGTTTCACCCAGGATGAATCAAGAACTGTGACAACCAATTTGATATCCCCATTCCAAGATCTGACCC
CAGATCTTACGGCTGAATTGCTCTGTATAATGAGGCTTTGGGGACACCCCATGCTCACCGCCAGTCAA
GCTGCGGGAAAGGTACGCGAGTCCATGTGTGCTGGAAAAGTATTAGACTTTCCCACCATTATGAAAAC
ACTAGCCTTTTTCCATACTATTCTGATCAATGGATACAGGAGGAAGCATCATGGAGTATGGCCACCCTT
AAACTTACCGGGTAATGCTTCAAAGGGTCTCACGGAACTTATGAATGACAATACTGAGATAAGCTATG
AATTCACACTTAAGCATTGGAAGGAAATCTCTCTTATAAAATTCAAGAAATGTTTTGATGCAGACGCA
GGTGAGGAACTCAGTATATTTATGAAAGATAAAGCAATTAGTGCCCCAAAACAAGATTGGATGAGTGT
GTTTAGAAGAAGCCTAATCAAACAGCGCCATCAGCATCATCAGGTCCCCCTACCAAATCCATTCAATC
GACGGCTATTGCTAAACTTTCTCGGAGATGACAAATTCGACCCGAATGTGGAGCTACAGTATGTAACA
TCAGGTGAGTATCTACATGATGACACGTTTTGTGCATCATATTCACTAAAAGAGAAGGAAATTAAACC
TGATGGTCGAATTTTTGCAAAGTTGACTAAGAGAATGAGATCATGTCAAGTTATAGCAGAATCTCTTTT
AGCGAACCATGCTGGGAAGTTAATGAAAGAGAATGGTGTTGTGATGAATCAGCTATCATTAACAAAAT
CACTATTAACAATGAGTCAGATTGGAATAATATCCGAGAAAGCTAGAAAATCGACTCGAGATAACAT
AAATCAACCTGGTTTCCAGAATATCCAGAGAAATAAATCACATCACTCCAAGCAAGTCAATCAGCGAG
ATCCAAGTGATGACTTTGAATTGGCAGCATCTTTTTTAACTACTGATCTCAAAAAATATTGTTTACAAT
GGAGGTACCAGACAATTATCCCATTTGCTCAATCATTAAACAGAATGTATGGTTATCCTCATCTCTTTG
AGTGGATTCACTTACGGCTAATGCGTAGTACACTTTACGTGGGGGATCCCTTCAACCCACCAGCAGAT
ACCAGTCAATTTGATCTAGATAAAGTAATTAATGGAGATATCTTCATTGTATCACCCAGAGGTGGAAT
TGAAGGGCTATGTCAAAAGGCTTGGACAATGATATCTATCTCTGTGATAATTCTATCTGCCACAGAGTC
TGGCACACGAGTAATGAGTATGGTGCAGGGAGATAATCAAGCAATTGCTGTCACCACACGAGTACCA
AGGAGCCTGCCGACTCTTGAGAAAAAGACTATTGCTTTTAGATCTTGTAATCTATTCTTTGAGAGGTTA
AAATGTAATAATTTTGGATTAGGTCACCATTTGAAAGAACAAGAGACTATCATTAGTTCTCACTTCTTT
GTTTATAGCAAGAGAATATTCTATCAGGGGAGGATTCTAACGCAAGCCTTAAAAAATGCTAGTAAGCT
CTGCTTGACAGCTGATGTCCTAGGAGAATGTACCCAATCATCATGTTCTAATCTTGCAACTACTGTCAT
GAGGTTAACTGAGAATGGTGTTGAAAAAGATATCTGTTTCTACTTGAATATCTATATGACCATCAAAC
AGCTCTCCTATGATATCATCTTCCCTCAAGTGTCAATTCCTGGAGATCAGATCACATTAGAATACATAA
ATAATCCACACCTGGTATCACGATTGGCTCTTCTGCCATCCCAGCTAGGAGGTCTAAACTACCTGTCAT
GCAGTAGGCTGTTCAATCGAAACATAGGCGACCCGGTGGTTTCCGCAGTTGCAGATCTTAAGAGATTA
ATTAAATCAGGATGTATGGATTACTGGATCCTTTATAACTTATTAGGGAGAAAACCGGGAAACGGCTC
ATGGGCTACTTTAGCAGCTGACCCGTACTCAATCAATATAGAGTATCAATACCCCCCAACTACAGCTCT
TAAGAGGCACACCCAACAAGCTCTGATGGAACTCAGTACGAATCCAATGTTACGTGGCATATTCTCTG
ACAATGCACAGGCAGAAGAAAATAATCTTGCTAGATTTCTCCTGGATAGGGAGGTGATCTTTCCGCGT
GTAGCTCACATCATCATTGAGCAAACCAGTGTCGGGAGGAGAAAACAGATTCAAGGATATTTGGATTC
AACTAGATCGATAATGAGTAAATCACTAGAAATTAAGCCCTTGTCCAATAGGAAGCTTAATGAAATAC
TGGATTACAACATCAATTACCTAGCTTACAATTTGGCATTACTCAAGAATGCTATTGAACCTCCGACTT
ATTTGAAAGCAATGACTCTTGAAACATGTAGCATCGACATTGCAAGGAGCCTCCGGAAGCTCTCCTGG
GCCCCACTCTTGGGTGGGAGAAATCTTGAAGGATTAGAGACGCCAGATCCCATTGAAATTACTGCAGG
AGCATTAATTGTTGGATCGGGCTACTGTGAACAGTGTGCTGCAGGAGACAATCGATTCACATGGTTTTT
CTTGCCATCTGGTATCGAGATAGGAGGGGATCCCCGTGATAATCCTCCTATCCGTGTACCGTACATTGG
CTCCAGGACTGATGAGAGGAGGGTAGCCTCAATGGCATACATCAGGGGTGCCTCGAGTAGCCTAAAA
GCAGTTCTTAGACTGGCGGGAGTGTACATCTGGGCATTCGGAGATACTCTGGAGAATTGGATAGATGC
ACTGGATTTGTCTCACACTAGAGTTAACATCACACTTGAACAGCTGCAATCCCTCACCCCACTTCCAAC
CTCTGCCAATCTAACCCATCGGTTGGATGATGGCACAACTACCCTAAAGTTTACTCCTGCGAGCTCTTA
CACCTTTTCAAGTTTCACTCATATATCAAATGATGAGCAATACCTGACAATTAATGACAAAACTGCAG
ATTCAAATATAATCTACCAACAGTTAATGATCACTGGACTCGGAATTTTAGAAACATGGAATAATCCC
CCAATCAATAGAACATTCGAAGAATCTACCCTACATTTGCACACTGGTGCATCATGTTGTGTCCGACCT
GTGGACTCCTGCATCATCTCAGAAGCATTAACAGTCAAGCCACATATTACAGTACCGTACAGCAATAA
ATTTGTATTTGATGAAGACCCGCTATCTGAATATGAGACTGCAAAACTGGAATCGTTATCATTTCAAGC
CCAATTAGGCAACATTGATGCTGTAGATATGACAGGTAAATTAACATTATTGTCCCAATTCACTGCAA
GGCAGATTATTAATGCAATCACTGGACTCGATGAGTCTGTCTCTCTTACTAATGATGCCATTGTTGCAT
CAGACTATGTCTCCAATTGGATTAGTGAATGCATGTATACCAAATTAGATGAATTATTTATGTATTGTG
GGTGGGAACTACTATTGGAACTATCCTATCAAATGTATTATCTGAGGGTAGTTGGGTGGAGTAATATA
GTGGATTATTCTTACATGATCTTGAGAAGAATCCCGGGTGCAGCATTAAACAATCTGGCATCTACATTA
AGTCATCCAAAACTTTTCCGACGAGCTATCAACCTAGATATAGTTGCCCCCTTAAATGCTCCTCATTTT
GCATCTCTGGACTACATCAAGATGAGTGTGGATGCAATACTCTGGGGCTGTAAAAGAGTCATCAATGT
GCTCTCCAATGGAGGGGACTTAGAATTAGTTGTGACATCTGAAGATAGCCTTATTCTCAGTGACCGAT
CCATGAATCTCATTGCAAGGAAATTAACTTTATTATCACTGATTCACCATAATGGTTTGGAACTACCAA
AGATTAAGGGGTTCTCTCCTGATGAGAAGTGTTTCGCTTTGACAGAATTTTTGAGGAAAGTGGTGAAC
TCAGGGTTGAGTTCAATAGAGAACCTATCAAATTTTATGTACAATGTGGAGAACCCACGGCTTGCAGC
ATTCGCCAGCAACAATTACTACCTGACCAGAAAATTATTGAATTCAATACGAGATACTGAGTCGGGTC
AAGTAGCAGTCACCTCATATTATGAATCATTAGAATATATTGATAGTCTTAAGCTAACCCCACATGTGC
CTGGTACCTCATGCATTGAGGATGATAGTCTATGTACAAATGATTACATAATCTGGATCATAGAGTCTA
ATGCAAACTTGGAGAAGTATCCAATTCCAAATAGCCCTGAGGATGATTCCAATTTCCATAACTTTAAG
TTGAATGCTCCATCGCACCATACCTTACGCCCATTAGGGTTGTCATCAACTGCTTGGTATAAGGGTATA
AGCTGTTGCAGGTACCTTGAGCGATTAAAGCTACCACAAGGTGATCATTTATATATTGCAGAAGGTAG
TGGTGCCAGTATGACAATCATAGAATACCTATTCCCAGGAAGAAAGATATATTACAATTCTTTATTTAG
TAGTGGTGACAATCCCCCACAAAGAAATTATGCACCAATGCCTACTCAGTTCATTGAGAGTGTCCCAT
ACAAGCTCTGGCAAGCACACACAGATCAATATCCCGAGATTTTTGAGGACTTCATCCCTCTATGGAAC
GGAAATGCCGCCATGACTGACATAGGAATGACAGCTTGTGTAGAATTCATCATCAATCGAGTCGGCCC
AAGGACTTGCAGTTTAGTACATGTAGATTTGGAATCAAGTGCAAGCTTAAATCAACAATGCCTGTCAA
AGCCGATAATTAATGCTATCATCACTGCTACAACTGTTTTGTGCCCTCATGGGGTGCTTATTCTGAAAT
ATAGTTGGTTGCCATTTACTAGATTTAGTACTTTGATCACTTTCTTATGGTGCTACTTTGAGAGAATCAC
TGTTCTTAGGAGCACATATTCTGGTCCAGCTAATCATGAGGTTTATTTAATTTGTATCCTTGCCAACAA
CTTTGCATTCCAGACTGTCTCGCAGGCAACAGGAATGGCGATGACTTTAACCGATCAAGGGTTTACTTT
GATATCACCTGAAAGAATAAATCAGTATTGGGATGGTCACTTGAAGCAAGAACGTATCGTAGCAGAA
GCAATTGATAAGGTGGTTCTAGGAGAAGATGCTCTATTCAATTCGAGTGATAATGAATTAATTCTCAA
ATGTGGAGGGACACCAAATGCACGGAATCTTATCGATATCGAGCCAGTCGCAACTTTCATAGAATTTG
AACAACTGATCTGCACAATGTTGACAACCCACTTGAAGGAAATAATTGATATAACAAGGTCTGGAACC
CAGGATTATGAAAGTTTATTACTCACTCCTTACAATTTAGGTCTTCTTGGTAAAATCAGTACGATAGTG
AGATTATTAACAGAAAGGATTCTAAATCATACTATCAGGAATTGGTTGATCCTCCCACCTTCGCTCCGG
ATGATCGTGAAGCAGGACTTGGAATTCGGCATATTCAGGATTACTTCCATCCTCAATTCTGATCGGTTC
CTGAAGCTTTCTCCAAATAGGAAATACTTGATTACACAATTAACTGCAGGCTACATTAGGAAATTGAT
TGAGGGGGATTGTAATATCGATCTAACCAGACCTATCCAAAAACAAATCTGGAAAGCATTAGGTTGTG
TAGTCTATTGTCACGATCCAGTAGATCAAAGGGAATCAACAGAGTTTATTGATATAAATATTAATGAA
GAAATAGACCGCGGGATCGATGGCGAGGAAATCTAAATATATCAAGAATCAGAATTAGTTTAAGAAA
AAAGAAGAGGATTAATCTTGGTTTTCCCCTTGGT.

21. CVL113

The nucleic acid sequence for CVL116 is:

(SEQ ID NO: 80)
ACCAGGGGGAAAACGAAGTGGTGACTCAAATCATCGAAGACCCTCGAGATTACATAGGTCCGGAACC
TATGGCCTTCGTGACCGACCTCGAGTCAGAGTAGTTCAATAAGGACCTATCAAGTTTGGGCAATTTTTC
GTCCCCGACACAAAAATGTCATCCGTGCTTAAAGCATATGAGCGATTCACACTCACTCAAGAACTGCA
AGATCAGAGTGAGGAAGGTACAATCCCACCTACAACACTAAAACCGGTAATCAGGGTATTTATACTAA
CCTCTAATAACCCAGAGCTAAGATCCCGGCTTCTTCTATTCTGCCTACGGATTGTTCTCAGTAATGGTG
CAAGGGATTCCCATCGCTTTGGAGCATTACTTACAATGTTTTCGCTACCATCAGCCACAATGCTCAATC
ATGTCAAATTAGCTGACCAGTCACCAGAAGCTGATATCGAAAGGGTAGAGATCGATGGCTTTGAGGA
GGGATCATTCCGCTTAATCCCCAATGCTCGTTCAGGTATGAGCCGTGGAGAGATCAATGCCTATGCTG
CACTTGCAGAAGATCTACCTGACACACTAAACCATGCAACACCTTTTGTTGATTCCGAAGTCGAGGGA
ACTGCATGGGATGAGATTGAGACTTTCTTAGATATGTGTTACAGTGTCCTAATGCAGGCATGGATAGT
GACTTGCAAGTGCATGACTGCGCCAGACCAACCTGCTGCTTCTATTGAGAAACGCCTGCAAAAATATC
GTCAGCAAGGCAGGATCAACCCGAGATATCTCCTGCAACCGGAGGCTCGAAGAATAATCCAGAATGT
AATCCGAAAGGGAATGGTGGTCAGACATTTCCTCACCTTTGAACTGCAGCTTGCCCGAGCACAAAGCC
TTGTATCAAATAGGTATTATGCTATGGTAGGGGATGTTGGAAAGTATATAGAGAATTGTGGAATGGGA
GGCTTCTTTTTGACACTAAAATATGCATTAGGAACCAGATGGCCCACACTTGCTTTAGCTGCATTTTCA
GGAGAGCTAACAAAGCTAAAGTCCCTCATGGCATTATACCAGACCCTTGGTGAGCAGGCCCGATATTT
GGCCCTATTGGAGTCACCACATTTGATGGATTTTGCTGCAGCAAACTACCCACTGCTATATAGCTATGC
TATGGGAATAGGCTATGTGTTAGATGTCAACATGAGGAACTACGCTTTCTCCAGATCATACATGAATA
AGACATATTTCCAATTGGGAATGGAAACTGCAAGAAAACAACAGGGTGCAGTTGACATGAGGATGGC
AGAAGATCTCGGTCTAACTCAAGCCGAACGCACCGAGATGGCAAATACACTTGCCAAATTGACCACA
GCAAATCGAGGGGCAGACACCAGGGGAGGAGTCAACCCGTTCTCATCTATCACTGGGACAACTCAGG
TGCCCGCTGCAGCAACAGGTGACACATTCGAGAGTTACATGGCAGCGGATCGACTGAGGCAGAGATA
TGCTGATGCAGGCACCCACGATGATGAGATGCCACCATTGGAAGAGGAGGAAGAGGACGACACATCT
GCAGGTCCACGCACTGGACTAACTCTTGAACAAGTGGCCTTGGACATCCAGAACGCAGCAGTTGGAGC
TCCCATCCATACAGATGACCTGAATGCCGCACTGGGTGATCTTGACATCTAGACAATTCAGATCCCAA
TCTTAAATCGACACACCTAATTGACCAGTTAGATGGAACTACAGTGGATTCCATAAGGTTCCTGCCTAC
CATCGGCTTTTAAGAAAAAAATAGGCCCGGACGGGTTAGCAACAAGCGACTGCCGATGCCAATAACA
CAATCCACAATCTACAATGGATCCCACTGATCTGAGCTTCTCCCCAGATGAGATCAATAAGCTCATAG
AGACAGGCCTGAATACTGTGGAGTATTTTACTTCCCAACAAGTCACAGGAACATCCTCTCTTGGAAAG
AATACAATACCACCAGGGGTCACAGGACTACTAACCAATGCTGCAGAGGCAAAGATCCAAGAGTCAA
CCAACCATCAGAAGGGTTCAGTTGGTGGGGGCGCAAAACCAAAGAAACCGCGACCAAAAATTGCCAT
TGTGCCAGCAGATGACAAAACGGTGCCCGGAAAGCCGATCCCAAACCCTCTATTAGGTCTGGACTCCA
CCCCGAGCACCCAAACTGTGCTTGATCTAAGTGGGAAAACATTACCATCAGGATCCTATAAGGGGGTT
AAACTTGCGAAATTTGGAAAAGAAAATCTGATGACACGGTTCATCGAGGAACCCAGAGAGAATCCTA
TCGCAACCAATTCCCCCATCGATTTTAAGAGGGGCAGGGATACCGGCGGGTTCCATAGAAGGGAGTAC
TCAATCGGATGGGTGGGAGATGAAGTCAAGGTCACTGAGTGGTGCAATCCATCCTGTTCTCCAATCAC
CGCTGCAGCAAGGCGATTTGAATGCACTTGTCACCAGTGTCCAGTCACTTGCTCTGAATGTGAACGAG
ATACTTAATACAGTGAGAAATTTGGACTCTCGGATGAATCAACTGGAGACAAAAGTAGATCGCATTCT
CTCATCTCAGTCTCTAATCCAGACCATCAAGAATGACATAGTTGGACTTAAAGCAGGGATGGCTACTT
TAGAAGGAATGATTACAACTGTGAAAATCATGGACCCGGGAGTTCCCAGTAATGTTACTGTGGAAGAT
GTACGCAAGAAACTAAGTAACCATGCTGTTGTTGTGCCAGAATCATTCAATGATAGTTTCTTGACTCAA
TCTGAAGATGTAATTTCACTTGATGAGTTGGCTCGACCAACTGCAACAAGTGTTAAGAAGATTGTCAG
GAAGGTTCCTCCTCAGAAGGATCTGACTGGATTGAAGATCACACTAGAGCAATTGGCAAAGGATTGCA
TCAGCAAACCGAAGATGAGGGAAGAGTATCTCCTCAAAATCAACCAGGCTTCCAGTGAGGCTCAGCT
AATTGACCTCAAGAAAGCAATCATCCGCAGTGCAATTTGATCAAGAAACACCCAATTACACTACACTG
GTATGACACTGTACTAACCCTGAGGGTTTTAGAAAAAACGATTAACGATAAATAAGCCCGAACACTAC
ACACCACCCGAGGCAGCCATGCCATCCATCAGCATCCCCGCAGACCCCACCAATCCACGTCAATCAAT
AAAAGCGTTCCCAATTGTGATCAACCGTGATGGGGGTGAGAAAGGTCGCTTGGTTAAACAACTACGCA
CAACCTACTTGAATGACCTAGATACTCATGAGCCACTGGTGACATTCGTAAATACTTATGGATTCATCT
ACGAACAGGATCGGGGAAATACCATTGTCGGAGAGGATCAACATGGGAAGAAAAGAGAGGCTGTGA
CTGCTGCAATGGTTACCCTTGGATGTGGGCCTAATCTACCATCATTAGGGAATGTCCTGGGACAACTG
AGTGAATTCCAGGTCATTGTTAGGAAGACATCCAGCAAAGCGGAAGAGATGGTCTTTGAAATTGTTAA
GTATCCGAGAATATTTCGGGGTCATACATTAATCCAGAAAGGACTAGTCTGTGTCTCCGCAGAAAAAT
TTGTTAAGTCACCAGGGAAAGTACAATCTGGAATGGACTATCTCTTCATTCCGACATTTCTGTCAGTGA
CTTACTGTCCAGCTGCAATCAAATTTCAGGTACCTGGCCCCATGTTGAAAATGAGATCAAGATACACT
CAGAGCTTACAACTTGAACTAATGATAAGAATCCTGTGTAAGCCCGATTCGCCACTTATGAAGGTCCA
TATCCCTGACAAGGAAGGAAGAGGATGTCTTGTATCAGTATGGTTGCATGTATGCAATATCTTCAAAT
CAGGAAACAAGAATGGCAGTGAGTGGCAGGAATACTGGATGAGAAAGTGTGCTAACATGCAACTTGA
AGTGTCGATTGCAGATATGTGGGGACCAACTATCATAATTCATGCCAGAGGTCACATTCCCAAAAGTG
CTAAGTTGTTTTTTGGAAAGGGTGGATGGAGCTGCCATCCACTTCACGAAGTTGTTCCAAGTGTCACTA
AAACACTATGGTCCGTGGGCTGTGAGATTACAAAGGCGAAGGCAATAATACAAGAGAGTAGCATCTC
TCTTCTCGTGGAGACTACTGACATCATAAGTCCAAAAGTCAAAATTTCATCTAAGCATCGCCGCTTTGG
GAAATCAAATTGGGGTCTGTTCAAGAAAACCAAATCACTGCCCAACCTGACGGAGCTGGAATGAgctagc
CCTGCTATAGGCTATCCACTGCATCATCTCTTTTTAAGAAAAAAATAGGCCCGGACGGGTTAGCAACA
AGCGACTGCCGGTGCCAACAACACAATCCACAATCTACAGTCGACGCGGCCGCCACCatgttcgtgttc
ctggtgctgctgccactggtcagcagccagtgcgtgaatttcactacccggacccagctgcctcctgctt
atacaaacagcttcaccaggggagtgtactaccccgataaggtgttccgcagctctgtgctgcacagc
acccaggacttgtttcttcccttcttcagcaacgtgacctggttccacgccatccacgtgtccggcac
caacggcaccaaaagattcgccaatcctgtgctgcctttcaacgatggcgtgtactttgcctctac
agagaagtctaacatcatcagaggctggatcttcggcacaaccctggattctaaaacccagagcctg
ctgatcgtgaacaatgccaccaatgtagtgatcaaggtttgtgagttccagttctgcaacgacccctttc
tgggcgtttactaccacaagaacaacaaaagctggatggaaagcgagttcagggtgtactctagcgcca
acaactgtaccttcgagtacgtgagccagccttttctgatggacctggagggcaagcagggcaacttca
aaaacctgcgggagttcgtgtttaagaacatcgacggatacttcaagatttactccaagcacaccccaat
taacttggttagaggactgccccaaggcttcagcgccctggaacccctggtggacctgcctatcgg
catcaacatcacaagattccaaaccctgcatagaagctatctgacaccaggcgactctagctctggat
ggacagccggcgccgctgcttattacgtgggctacctgcagcctagaaccttcctcctgaaatacaac
gagaacggaaccatcaccgacgccgtggactgcgccctggatcctctgagcgagacaaagtgcaccctg
aagtccttcaccgtcgaaaagggcatctaccaaacaagcaacttcagagtgcagcctactgagagcatcg
tgcggtttccaaacatcaccaacctgtgtccttttggcgaggtgttcaacgccacccggttcgcttccgt
gtacgcctggaatagaaagcggatcagcaactgcgtggccgactacagcgtgctgtataatagcgcc
tcattttctacttttaaatgttacggcgtgtctcctacaaagctgaacgatctgtgcttcacaaatgt
gtacgccgatagcttcgtgattagaggcgacgaggtgcggcagatcgcccctggccagaccggcaac
atcgctgactacaactacaagctgcctgacgacttcaccggctgtgtgatcgcatggaacagcaata
acctggactctaaggtgggcggaaactacaattacctgtacagactgttcagaaagagcaacctgaagc
ccttcgagagagatatctccacagaaatctatcaggccggatctacaccttgtaatggcgtgaagggctt
caactgctacttccctctgcaatcctacggctttcagcccacatacggcgtgggctaccagccataccgg
gtcgtggtgctgagcttcgagctgctgcacgcccctgccacagtgtgcggccctaagaagagcacc
aatctggtaaagaacaagtgcgtcaacttcaatttcaacggcctgaccgggaccggagtgctgaccg
aaagcaacaagaaattcctgccgttccagcagttcggcagagatatcgccgacaccaccgatgccgt
gcgggacccccagacacttgaaatcctggacatcaccccttgcagttttggcggcgtgtccgtgatca
cacctggaacaaacaccagcaaccaggtggccgtgctgtaccagggcgtcaactgcaccgaggtccc
cgtggccatccacgccgatcagctgacacctacatggcgggtgtactctaccggcagcaacgtgttcca
aaccagagccggctgcctgatcggcgctgagcacgtgaacaacagctacgagtgcgacattcctatcgg
tgctggcatctgcgcctcttaccagacacagacaaacagccccagaagagcgagaagcgtggctagtcag
agcatcatcgcctacaccatgagcctgggcgtggagaacagcgtggcctactctaacaatagcatcgcca
tcccaaccaacttcacaatctctgtgaccacagagatcctgcccgtgtccatgaccaagacatctgtg
gactgcacaatgtatatctgtggcgattctaccgagtgcagcaacctgctgctccagtacggcagcttt
tgtacccagctgaatagagccctgacaggcatcgccgtggagcaggataagaacacacaggaggtgttc
gcccaggtgaagcaaatctacaagaccccccctatcaaggactttggcggcttcaatttttcccagatc
ctgcctgatccatccaagccttctaagcggagctttatcgaggacctgctgttcaacaaggtgacc
ctggccgatgccggcttcatcaagcagtatggcgattgcctgggcgacatcgcagccagggacctg
atctgcgcccagaagtttaatggcctgaccgtgctgccacccctgctgacagatgagatgatcgc
acagtacacaagcgccctgctggccggcaccatcacatccggatggaccttcggcgcaggagccgcc
ctccagatcccctttgccatgcagatggcctataggttcaacggcatcggcgtgacccagaatgtgct
gtacgagaaccagaagctgatcgccaatcagtttaactccgccatcggcaagatccaggacagcctg
tcctctacagccagcgccctgggcaagctccaggatgtggtgaatcagaacgcccaggccctgaata
ccctggtgaagcagctgagcagcaacttcggcgccatctctagcgtgctgaatgacatcctgagccgg
ctggacaaggtggaggcagaggtgcagatcgaccggctgatcaccggccggctccagagcctccagacct
atgtgacacagcagctgatcagggccgccgagatcagggccagcgccaatctggcagcaaccaagatgtc
cgagtgcgtgctgggccagtctaagagagtggacttttgtggcaagggctatcacctgatgtccttcc
ctcagtctgccccacacggcgtggtgtttctgcacgtgacctacgtgcccgcccaggagaagaac
ttcaccacagcccctgccatctgccacgatggcaaggcccactttccaagggagggcgtgttcgtg
tccaacggcacccactggtttgtgacacagcgcaatttctacgagccccagatcatcaccacaga
caacaccttcgtgagcggcaactgtgacgtggtcatcggcatcgtgaacaataccgtgtatgatc
cactccagcccgagctggacagctttaaggaggagctggataagtatttcaagaatcacacctcccc
tgacgtggatctgggcgacatcagcggcatcaatgcctccgtggtgaacatccagaaggagatcgac
cgcctgaacgaggtggctaagaatctgaacgagagcctgatcgacctccaggagctgggcaagtatga
gcagtacatcaagtggccctggtacatctggctgggcttcatcgccggcctgatcgccatcgtgatggt
gaccatcatgctgtgctgtatgacatcctgctgttcttgcctgaagggctgctgtagctgtggctcctg
ctgtAAGTTATTGACCATTGTCGTTGCTAATCGAAATAGAATGGAGAATTTTGTTTATCATAAATGACGT
ACGAGGCAGAAGCCTCCAGGTCTGACCCAGCCAATCAAAGCATTATACCAGACCATGGAATGCATAC
CAAACATTATTGACACTAATGACACACAAAATTGGTTTTAAGAAAAACCAAGAGAACAATAGGCCAG
AATGGCTGGGTCTCGGGAGATATTACTCCCTGAAGTCCATCTCAATTCACCAATTGTAAAGCATAAGC
TATACTATTACATTCTACTTGGAAACCTCCCAAATGAGATCGACATTGACGATTTAGGTCCATTACATA
ATCAAAATTGGAATCAAATAGCACATGAAGAGTCTAACTTAGCCCAACGCTTGGTAAATGTAAGAAAT
TTTCTAATTACCCACATCCCTGATCTTAGAAAGGGCCATTGGCAAGAGTATGTCAATGTAATACTGTGG
CCGCGAATTCTTCCCTTGATCCCGGATTTTAAAATCAATGACCAATTGCCTCTACTCAAAAATTGGGAC
AAGTTAGTTAAAGAATCATGTTCAGTAATCAATGCGGGTACTTCCCAGTGCATTCAGAATCTCAGCTAT
GGACTGACAGGTCGTGGGAACCTCTTTACACGATCACGTGAACTCTCTGGTGACCGCAGGGATATTGA
TCTTAAGACGGTTGTGGCAGCATGGCATGACTCAGACTGGAAAAGAATAAGTGATTTTTGGATTATGA
TCAAATTCCAGATGAGACAATTAATTGTTAGGCAAACAGATCATAATGATCCTGATTTAATCACGTAT
ATCGAAAATAGAGAAGGCATAATCATCATAACCCCTGAACTGGTAGCATTATTTAACACTGAGAATCA
TACACTAACATACATGACCTTTGAAATTGTACTGATGGTTTCAGATATGTACGAAGGTCGTCACAACAT
TTTATCACTATGCACAGTTAGCACTTACCTGAATCCTCTGAAGAAAAGAATAACATATTTATTGAGCCT
TGTAGATAACTTAGCTTTTCAGATAGGTGATGCTGTATATAACATAATTGCTTTGCTAGAATCCTTTGT
ATATGCACAGTTGCAAATGTCAGATCCCATCCCAGAACTCAGAGGACAATTCCATGCATTCGTATGTT
CTGAGATTCTTGATGCACTAAGGGGAACTAATAGTTTCACCCAGGATGAATCAAGAACTGTGACAACC
AATTTGATATCCCCATTCCAAGATCTGACCCCAGATCTTACGGCTGAATTGCTCTGTATAATGAGGCTT
TGGGGACACCCCATGCTCACCGCCAGTCAAGCTGCGGGAAAGGTACGCGAGTCCATGTGTGCTGGAA
AAGTATTAGACTTTCCCACCATTATGAAAACACTAGCCTTTTTCCATACTATTCTGATCAATGGATACA
GGAGGAAGCATCATGGAGTATGGCCACCCTTAAACTTACCGGGTAATGCTTCAAAGGGTCTCACGGAA
CTTATGAATGACAATACTGAGATAAGCTATGAATTCACACTTAAGCATTGGAAGGAAATCTCTCTTAT
AAAATTCAAGAAATGTTTTGATGCAGACGCAGGTGAGGAACTCAGTATATTTATGAAAGATAAAGCA
ATTAGTGCCCCAAAACAAGATTGGATGAGTGTGTTTAGAAGAAGCCTAATCAAACAGCGCCATCAGCA
TCATCAGGTCCCCCTACCAAATCCATTCAATCGACGGCTATTGCTAAACTTTCTCGGAGATGACAAATT
CGACCCGAATGTGGAGCTACAGTATGTAACATCAGGTGAGTATCTACATGATGACACGTTTTGTGCAT
CATATTCACTAAAAGAGAAGGAAATTAAACCTGATGGTCGAATTTTTGCAAAGTTGACTAAGAGAATG
AGATCATGTCAAGTTATAGCAGAATCTCTTTTAGCGAACCATGCTGGGAAGTTAATGAAAGAGAATGG
TGTTGTGATGAATCAGCTATCATTAACAAAATCACTATTAACAATGAGTCAGATTGGAATAATATCCG
AGAAAGCTAGAAAATCGACTCGAGATAACATAAATCAACCTGGTTTCCAGAATATCCAGAGAAATAA
ATCACATCACTCCAAGCAAGTCAATCAGCGAGATCCAAGTGATGACTTTGAATTGGCAGCATCTTTTTT
AACTACTGATCTCAAAAAATATTGTTTACAATGGAGGTACCAGACAATTATCCCATTTGCTCAATCATT
AAACAGAATGTATGGTTATCCTCATCTCTTTGAGTGGATTCACTTACGGCTAATGCGTAGTACACTTTA
CGTGGGGGATCCCTTCAACCCACCAGCAGATACCAGTCAATTTGATCTAGATAAAGTAATTAATGGAG
ATATCTTCATTGTATCACCCAGAGGTGGAATTGAAGGGCTATGTCAAAAGGCTTGGACAATGATATCT
ATCTCTGTGATAATTCTATCTGCCACAGAGTCTGGCACACGAGTAATGAGTATGGTGCAGGGAGATAA
TCAAGCAATTGCTGTCACCACACGAGTACCAAGGAGCCTGCCGACTCTTGAGAAAAAGACTATTGCTT
TTAGATCTTGTAATCTATTCTTTGAGAGGTTAAAATGTAATAATTTTGGATTAGGTCACCATTTGAAAG
AACAAGAGACTATCATTAGTTCTCACTTCTTTGTTTATAGCAAGAGAATATTCTATCAGGGGAGGATTC
TAACGCAAGCCTTAAAAAATGCTAGTAAGCTCTGCTTGACAGCTGATGTCCTAGGAGAATGTACCCAA
TCATCATGTTCTAATCTTGCAACTACTGTCATGAGGTTAACTGAGAATGGTGTTGAAAAAGATATCTGT
TTCTACTTGAATATCTATATGACCATCAAACAGCTCTCCTATGATATCATCTTCCCTCAAGTGTCAATTC
CTGGAGATCAGATCACATTAGAATACATAAATAATCCACACCTGGTATCACGATTGGCTCTTCTGCCAT
CCCAGCTAGGAGGTCTAAACTACCTGTCATGCAGTAGGCTGTTCAATCGAAACATAGGCGACCCGGTG
GTTTCCGCAGTTGCAGATCTTAAGAGATTAATTAAATCAGGATGTATGGATTACTGGATCCTTTATAAC
TTATTAGGGAGAAAACCGGGAAACGGCTCATGGGCTACTTTAGCAGCTGACCCGTACTCAATCAATAT
AGAGTATCAATACCCCCCAACTACAGCTCTTAAGAGGCACACCCAACAAGCTCTGATGGAACTCAGTA
CGAATCCAATGTTACGTGGCATATTCTCTGACAATGCACAGGCAGAAGAAAATAATCTTGCTAGATTT
CTCCTGGATAGGGAGGTGATCTTTCCGCGTGTAGCTCACATCATCATTGAGCAAACCAGTGTCGGGAG
GAGAAAACAGATTCAAGGATATTTGGATTCAACTAGATCGATAATGAGTAAATCACTAGAAATTAAGC
CCTTGTCCAATAGGAAGCTTAATGAAATACTGGATTACAACATCAATTACCTAGCTTACAATTTGGCAT
TACTCAAGAATGCTATTGAACCTCCGACTTATTTGAAAGCAATGACTCTTGAAACATGTAGCATCGAC
ATTGCAAGGAGCCTCCGGAAGCTCTCCTGGGCCCCACTCTTGGGTGGGAGAAATCTTGAAGGATTAGA
GACGCCAGATCCCATTGAAATTACTGCAGGAGCATTAATTGTTGGATCGGGCTACTGTGAACAGTGTG
CTGCAGGAGACAATCGATTCACATGGTTTTTCTTGCCATCTGGTATCGAGATAGGAGGGGATCCCCGT
GATAATCCTCCTATCCGTGTACCGTACATTGGCTCCAGGACTGATGAGAGGAGGGTAGCCTCAATGGC
ATACATCAGGGGTGCCTCGAGTAGCCTAAAAGCAGTTCTTAGACTGGCGGGAGTGTACATCTGGGCAT
TCGGAGATACTCTGGAGAATTGGATAGATGCACTGGATTTGTCTCACACTAGAGTTAACATCACACTT
GAACAGCTGCAATCCCTCACCCCACTTCCAACCTCTGCCAATCTAACCCATCGGTTGGATGATGGCAC
AACTACCCTAAAGTTTACTCCTGCGAGCTCTTACACCTTTTCAAGTTTCACTCATATATCAAATGATGA
GCAATACCTGACAATTAATGACAAAACTGCAGATTCAAATATAATCTACCAACAGTTAATGATCACTG
GACTCGGAATTTTAGAAACATGGAATAATCCCCCAATCAATAGAACATTCGAAGAATCTACCCTACAT
TTGCACACTGGTGCATCATGTTGTGTCCGACCTGTGGACTCCTGCATCATCTCAGAAGCATTAACAGTC
AAGCCACATATTACAGTACCGTACAGCAATAAATTTGTATTTGATGAAGACCCGCTATCTGAATATGA
GACTGCAAAACTGGAATCGTTATCATTTCAAGCCCAATTAGGCAACATTGATGCTGTAGATATGACAG
GTAAATTAACATTATTGTCCCAATTCACTGCAAGGCAGATTATTAATGCAATCACTGGACTCGATGAGT
CTGTCTCTCTTACTAATGATGCCATTGTTGCATCAGACTATGTCTCCAATTGGATTAGTGAATGCATGT
ATACCAAATTAGATGAATTATTTATGTATTGTGGGTGGGAACTACTATTGGAACTATCCTATCAAATGT
ATTATCTGAGGGTAGTTGGGTGGAGTAATATAGTGGATTATTCTTACATGATCTTGAGAAGAATCCCG
GGTGCAGCATTAAACAATCTGGCATCTACATTAAGTCATCCAAAACTTTTCCGACGAGCTATCAACCT
AGATATAGTTGCCCCCTTAAATGCTCCTCATTTTGCATCTCTGGACTACATCAAGATGAGTGTGGATGC
AATACTCTGGGGCTGTAAAAGAGTCATCAATGTGCTCTCCAATGGAGGGGACTTAGAATTAGTTGTGA
CATCTGAAGATAGCCTTATTCTCAGTGACCGATCCATGAATCTCATTGCAAGGAAATTAACTTTATTAT
CACTGATTCACCATAATGGTTTGGAACTACCAAAGATTAAGGGGTTCTCTCCTGATGAGAAGTGTTTCG
CTTTGACAGAATTTTTGAGGAAAGTGGTGAACTCAGGGTTGAGTTCAATAGAGAACCTATCAAATTTT
ATGTACAATGTGGAGAACCCACGGCTTGCAGCATTCGCCAGCAACAATTACTACCTGACCAGAAAATT
ATTGAATTCAATACGAGATACTGAGTCGGGTCAAGTAGCAGTCACCTCATATTATGAATCATTAGAAT
ATATTGATAGTCTTAAGCTAACCCCACATGTGCCTGGTACCTCATGCATTGAGGATGATAGTCTATGTA
CAAATGATTACATAATCTGGATCATAGAGTCTAATGCAAACTTGGAGAAGTATCCAATTCCAAATAGC
CCTGAGGATGATTCCAATTTCCATAACTTTAAGTTGAATGCTCCATCGCACCATACCTTACGCCCATTA
GGGTTGTCATCAACTGCTTGGTATAAGGGTATAAGCTGTTGCAGGTACCTTGAGCGATTAAAGCTACC
ACAAGGTGATCATTTATATATTGCAGAAGGTAGTGGTGCCAGTATGACAATCATAGAATACCTATTCC
CAGGAAGAAAGATATATTACAATTCTTTATTTAGTAGTGGTGACAATCCCCCACAAAGAAATTATGCA
CCAATGCCTACTCAGTTCATTGAGAGTGTCCCATACAAGCTCTGGCAAGCACACACAGATCAATATCC
CGAGATTTTTGAGGACTTCATCCCTCTATGGAACGGAAATGCCGCCATGACTGACATAGGAATGACAG
CTTGTGTAGAATTCATCATCAATCGAGTCGGCCCAAGGACTTGCAGTTTAGTACATGTAGATTTGGAAT
CAAGTGCAAGCTTAAATCAACAATGCCTGTCAAAGCCGATAATTAATGCTATCATCACTGCTACAACT
GTTTTGTGCCCTCATGGGGTGCTTATTCTGAAATATAGTTGGTTGCCATTTACTAGATTTAGTACTTTGA
TCACTTTCTTATGGTGCTACTTTGAGAGAATCACTGTTCTTAGGAGCACATATTCTGGTCCAGCTAATC
ATGAGGTTTATTTAATTTGTATCCTTGCCAACAACTTTGCATTCCAGACTGTCTCGCAGGCAACAGGAA
TGGCGATGACTTTAACCGATCAAGGGTTTACTTTGATATCACCTGAAAGAATAAATCAGTATTGGGAT
GGTCACTTGAAGCAAGAACGTATCGTAGCAGAAGCAATTGATAAGGTGGTTCTAGGAGAAGATGCTCT
ATTCAATTCGAGTGATAATGAATTAATTCTCAAATGTGGAGGGACACCAAATGCACGGAATCTTATCG
ATATCGAGCCAGTCGCAACTTTCATAGAATTTGAACAACTGATCTGCACAATGTTGACAACCCACTTG
AAGGAAATAATTGATATAACAAGGTCTGGAACCCAGGATTATGAAAGTTTATTACTCACTCCTTACAA
TTTAGGTCTTCTTGGTAAAATCAGTACGATAGTGAGATTATTAACAGAAAGGATTCTAAATCATACTAT
CAGGAATTGGTTGATCCTCCCACCTTCGCTCCGGATGATCGTGAAGCAGGACTTGGAATTCGGCATATT
CAGGATTACTTCCATCCTCAATTCTGATCGGTTCCTGAAGCTTTCTCCAAATAGGAAATACTTGATTAC
ACAATTAACTGCAGGCTACATTAGGAAATTGATTGAGGGGGATTGTAATATCGATCTAACCAGACCTA
TCCAAAAACAAATCTGGAAAGCATTAGGTTGTGTAGTCTATTGTCACGATCCAGTAGATCAAAGGGAA
TCAACAGAGTTTATTGATATAAATATTAATGAAGAAATAGACCGCGGGATCGATGGCGAGGAAATCTA
AATATATCAAGAATCAGAATTAGTTTAAGAAAAAAGAAGAGGATTAATCTTGGTTTTCCCCTTGGT.

### C. Methods of Making

Also included in the present invention are methods of making and using PIV5 viral expression vectors, including, but not limited to any of those described herein.

For example, the present invention includes methods of expressing a coronavirus S protein, including but not limited to the S protein of SARS-CoV-2, in a cell by contacting or infection the cell with a PIV5 viral expression vector, viral particle, or composition as described herein.

The present invention includes methods of inducing an immune response in a subject to a coronavirus S protein, including but not limited to the S protein of SARS-CoV-2, by administering a viral expression vector, viral particle, or composition as described herein to the subject. The immune response may include a humoral immune response and/or a cellular immune response. The immune response may enhance an innate and/or adaptive immune response.

The present invention includes methods expressing a heterologous coronavirus S protein, including but not limited to the S protein of SARS-CoV-2, in a subject by administering a viral expression vector, viral particle, or composition as described herein to the subject.

The present invention includes methods expressing a heterologous coronavirus S protein, including but not limited to the S protein of SARS-CoV-2 alpha, gamma, delta, and omicron strains, in a subject by administering a viral expression vector, viral particle, or composition as described herein to the subject.

### D. Methods of Treatment

The disclosure can be used in gene therapy and/or therapeutic approaches for the treatment of disease which involve the increase or decrease of a nucleotide sequence of interest in a host-cell. In these embodiments, the expressible heterologous nucleotide sequence may be derived from a mammalian genome. It may be particularly useful in some embodiments to have the expressible heterologous nucleotide sequence derived from a human genome, wherein expression of the wild-type RNA and/or protein can produce therapeutic effects in a patient. For example, the expressible heterologous nucleotide sequence can encode CFTR, NeuroD1, Cas9 and Guide RNAs, or any other such sequence. In other embodiments, the heterologous nucleotide sequence encodes a secreted protein.

In other embodiments, the expressible heterologous nucleotide sequence responds to positive selection stimuli. In other embodiments, the expressible heterologous nucleotide sequence also responds to negative selection stimuli. In further embodiments, it may be useful for the polynucleotide sequences to further comprise a reporter gene. For example, the report gene can be a luciferase or green fluorescent protein.

In some embodiments, AVLP expresses one or more nucleotide sequences (e.g., siRNAs) that modify the translation and/or transcription of a host-cell nucleotide sequence of interest within a host cell. In some embodiments, transcription and/or translation of the expressible heterologous nucleotide sequence is modified so that its nucleotide sequence is codon degenerated with respect to the endogenous gene in a cell. Additionally, the expressible heterologous nucleotide sequence can be modified so that it co-expresses inhibitory or silencing sequences capable of inhibiting or silencing a host-cell nucleotide sequence of interest within a host cell.

In other embodiments, the expressible heterologous nucleotide of interest generates a product that stabilizes host-cell RNA nucleotide sequences. Such a product can be inducible or continually expressed. For example, the 3′ RhoB untranslated region (UTR) can stabilize target RNAs that express either toxic proteins or other proteins of interest in response to serum. Another example is linking the eotaxin 3′ untranslated region to the target gene of interest, which normally has a low half-life, but is stabilized with the addition of TNF-alpha and IL-4 to the cells. Alternatively, sequences contained in 16 mer sequence in the 5′ coding region of CYP2E 1 and CYP2B 1 mRNA destabilizes target RNAs in the presence of insulin. Upon the removal of insulin the target RNAs are stabilized and the proteins can be expressed (Trong et al., Biochem J., Dec. 23, 2004).

Further non-limiting examples of expressible heterologous sequences that can be used in the invented compositions and methods include sequences can produce proteins, including, for example, e.g., interferons (alpha, beta, gamma, epsilon), erythropoietin, Factor VIII, clotting factors, antibodies and fragments thereof (e.g., including single chain, Fab, and humanized), insulin, chemokines, cytokines, growth factors, angiogenesis modulatory factors, apoptosis modulatory factors, e.g., Growth Factors, including, e.g., Amphiregulin, B-lymphocyte stimulator, Interleukin 16 (IL16), Thymopoietin, TRAIL, Apo-2, Pre B cell colony enhancing factor, Endothelial differentiation-related factor 1 (EDF1), Endothelial monocyte activating polypeptide II, Macrophage migration inhibitory factor MIF, Natural killer cell enhancing factor (NKEFA), Bone morphogenetic protein 8 (osteogenic protein 2), Bone morphogenic protein 6, Connective tissue growth factor (CTGF), CGI-149 protein (neuroendocrine differentiation factor), Cytokine A3 (macrophage inflammatory protein 1-alpha), Glialblastoma cell differentiation-related protein (GBDR1), Hepatoma-derived growth factor, Neuromedin U-25 precursor, any tumor gene, oncogene, proto-oncogene or cell modulating gene (which can be found at condor.bcm.tmc.edu/oncogene), Vascular endothelial growth factor (VEGF), Vascular endothelial growth factor B (VEGF-B), T-cell specific RANTES precursor, Thymic dendritic cell-derived factor 1; Receptors, such as Activin A receptor, type II (ACVR2), β-signal sequence receptor (SSR2), CD14 monocyte LPS receptor, CD36 (collagen type 1/thrombospondin receptor)-like 2, CD44R (Hermes antigen gp90 homing receptor), G protein coupled receptor 9, Chemokine CXC receptor 4, Colony stimulating factor 2 receptor β(CSF2RB), FLT-3 receptor tyrosine kinase, Similar to transient receptor potential C precursor, Killer cell lectin-like receptor subfamily B, Low density lipoprotein receptor gene, low-affinity Fc-gamma receptor IIC, MCP-1 receptor, Monocyte chemoattractant protein 1 receptor (CCR2), Nuclear receptor subfamily 4, group A, member 1, Orphan G protein-coupled receptor GPRC5D, Peroxisome proliferative activated receptor gamma, Pheromore related-receptor (rat), Vasopressin-activated calcium mobilizing putative receptor, Retinoic×receptor, Toll-like receptor 6, Transmembrane activator and CAML interactor (TACI), B cell maturation peptide (BCMA), CSF-1 receptor, Interferon (α, β and gamma) receptor 1 (IFNAR1). Pathways that can be modulated to increase antibody production include, e.g., ubiquitin/proteosome; telomerase; FGFR3; and Mcd-1, etc.

In certain embodiments of the disclosure, AVLP compositions can be utilized to prepare antigenic preparations that be used as vaccines. Any suitable antigen(s) can be prepared in accordance with the disclosure, including antigens obtained from prions, viruses, mycobacterium, protozoa (e.g., Plasmodium falciparum (malaria)), trypanosomes, bacteria (e.g., Streptococcus, Neisseria, etc.), etc.

Host cells can be transfected with single AVLP particles containing one or more heterologous polynucleotide sequences, or with a plurality of AVLP particles, where each comprises the same or different heterologous polynucleotide sequence(s). For example, a multi-subunit antigen (including intracellular and cell-surface multi-subunit components) can be prepared by expressing the individual subunits on separate vectors, but infecting the same host cell with all the vectors, such that assembly occurs within the host cell.

Vaccines often contain a plurality of antigen components, e.g., derived from different proteins, and/or from different epitopic regions of the same protein. For example, a vaccine against a viral disease can comprise one or more polypeptide sequences obtained from the virus which, when administered to a host, elicit an immunogenic or protective response to viral challenge.

As mentioned, the disclosure can also be utilized to prepare polypeptide multimers, e.g., where an antigenic preparation is produced which is comprised of more than one polypeptide. For instance, virus capsids can be made up of more than one polypeptide subunit. By transducing a host cell with vectors carrying different viral envelope sequences, the proteins, when expressed in the cell, can self-assemble into three-dimensional structures containing more than one protein subunit (e.g., in their native configuration).

In further embodiments, the expressible heterologous nucleotide sequence is derived from another virus, other than PIV5. For example, the heterologous nucleotide sequence may encode (from any strain) influenza HA, RSV F, HIV Gag and/or Env, etc. Such embodiments can be useful for developing vaccines and/or methods of vaccination. The examples given here are non-limiting, as it will be understood by those in the art that nucleotide sequences from a variety of pathogenic agents (including also bacteria, parasites, etc.) may be desirable to use for an AVLP vaccine composition and/or method of vaccination.

Examples of viruses to which vaccines can be produced in accordance with the disclosure include, e.g., coronaviruses, orthomyxoviruses, influenza virus A (including all strains varying in their HA and NA proteins, such as (non-limiting examples) H1N1, H1N2, H2N2, H3N2, H7N7, and H3N8); influenza B, influenza C, thogoto virus (including Dhori, Batken virus, SiAR 126 virus), and isavirus (e.g., infectious salmon anemia virus) and the like. These include coronaviruses isolated or transmitted from all species types, including isolates from invertebrates, vertebrates, mammals, humans, non-human primates, monkeys, pigs, cows, and other livestock, birds, domestic poultry such as turkeys, chickens, quail, and ducks, wild birds (including aquatic and terrestrial birds), reptiles, etc. These also include existing strains which have changed, e.g., through mutation, antigenic drift, antigenic shift, recombination, etc., especially strains which have increased virulence and/or interspecies transmission (e.g., human-to-human).

### E. Methods of Administration

I. Administration by Vaccination

The present invention includes methods of vaccinating a subject by administering a viral expression vector, viral particle, or composition as described herein to the subject.

The disclosure provides vaccines against all coronaviruses, including existing subtypes, derivatives thereof, and recombinants thereof, such as subtypes and recombinants which have the ability to spread from human-to-human. Various isolates have been characterized, especially for SARS-COV-2.

The disclosure also provides methods for producing AVLP compositions. Examples of host cells which can be utilized to produce AVLP compositions, include, any mammalian or human cell line or primary cell. Non-limiting examples include, e.g., 293, HT1080, Jurkat, and SupT1 cells. Other examples are CHO, 293, Hela, Vero, L929, BHK, NIH 3T3, MRC-5, BAE-1, HEP-G2, NSO, U937, Namalwa, HL60, WEHI 231, YAC 1, U 266B1, SH-SY5Y, CHO, e.g., CHO-K1 (CCL-61), 293 (e.g., CRL-1573). Cells are cultured under conditions effective to produce transfection and expression. Such conditions include, e.g., the particular milieu needed to achieve protein production. Such a milieu, includes, e.g., appropriate buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including cell media, substrates, oxygen, carbon dioxide, glucose and other sugar substrates, serum, growth factors, etc.).

The disclosure also provides various treatment methods involving delivering AVLP to host cells in vivo. In some embodiments, AVLP is delivered into a subject for treating or preventing coronaviruses. In other embodiments, AVLP is delivered into a subject for treating or preventing SARS-COV-2 alpha, delta, omicron strains, or variants thereof or eliciting an immune response to SARS-COV-2 in a subject.

It is contemplated that when used to treat various diseases, the compositions and methods of the disclosure can be combined with other therapeutic agents suitable for the same or similar diseases. Also, two or more embodiments of the disclosure may be also co-administered to generate additive or synergistic effects. When co-administered with a second therapeutic agent, the embodiment of the disclosure and the second therapeutic agent may be simultaneously or sequentially (in any order). Suitable therapeutically effective dosages for each agent may be lowered due to the additive action or synergy.

As a non-limiting example, the disclosure can be combined with other therapies that block inflammation through (e.g., via inhibition, reduction and/or blockage of ILL INFα/β, IL6, TNF, L13, IL23, etc.). In some embodiments, AVLP compositions and methods disclosed herein are useful to enhance the efficacy of vaccines directed to SARS-COV-2 infections. The compositions and methods of the disclosure can be administered to a subject either simultaneously with or before (e.g., 1-30 days before) a reagent (including but not limited to small molecules, antibodies, or cellular reagents) that acts to elicit an immune response (e.g., to treat cancer or an infection). The compositions and methods of the disclosure can be also administered in combination with an anti-tumor antibody or an antibody directed at a pathogenic antigen or allergen.

The pharmaceutical compositions of the invention can be readily employed in a variety of therapeutic or prophylactic applications, e.g., for treating SARS-COV-2 infection or eliciting an immune response to SARS-COV-2 in a subject. In various embodiments, the vaccine compositions can be used for treating or preventing infections caused by a pathogen from which the displayed immunogen polypeptide in the PIV5-based vaccine is derived. Thus, the vaccine compositions of the invention can be used in diverse clinical settings for treating or preventing infections caused by various viruses. As exemplification, a SARS-COV-2 PIV-5-based vaccine composition can be administered to a subject to induce an immune response to SARS-COV-2, e.g., to induce production of broadly neutralizing antibodies to the virus. For subjects at risk of developing an SARS-COV-2 infection, a vaccine composition of the invention can be administered to provide prophylactic protection against viral infection. Therapeutic and prophylactic applications of vaccines derived from the other immunogens described herein can be similarly performed. Depending on the specific subject and conditions, pharmaceutical compositions of the invention can be administered to subjects by a variety of administration modes known to the person of ordinary skill in the art, for example, topical, oral, intranasal, intramuscular, subcutaneous, intravenous, intra-arterial, intra-articular, intraperitoneal, or parenteral routes. In some aspects, administration is to a mucosal surface. A vaccine may be administered by mass administration techniques such as by placing the vaccine in drinking water or by spraying the animals' environment. When administered by injection, the immunogenic composition or vaccine may be administered parenterally. Parenteral administration includes, for example, administration by intravenous, subcutaneous, intramuscular, or intraperitoneal injection.

1. Vaccination by Inhalation

In one embodiment, the disclosed PIV-5 vaccine compositions are formulated to allow intranasal administration. Intranasal compositions may comprise an inhalable dry powder pharmaceutical formulation comprising a therapeutic agent, wherein the therapeutic agent is present as a freebase or as a mixture of a salt and a freebase. Pharmaceutical formulations disclosed herein can be formulated as suitable for airway administration, for example, nasal, intranasal, sinusoidal, peroral, and/or pulmonary administration. Typically, formulations are produced such that they have an appropriate particle size for the route, or target, of airway administration. As such, the formulations disclosed herein can be produced so as to be of defined particle size distribution.

For example, the particle size distribution for a salt form of a therapeutic agent for intranasal administration can be between about 5 μm and about 350 More particularly, the salt form of the therapeutic agent can have a particle size distribution for intranasal administration between about 5μ to about 250 μm, about 10 μm to about 200 μm, about 15 μm to about 150 μm, about 20 μm to about 100 μm, about 38 μm to about 100 μm, about 53 μm to about 100, about 53 μm to about 150 μm, or about 20 μm to about 53 μm. The salt form of the therapeutic agent in the pharmaceutical compositions of the invention can a particle size distribution range for intranasal administration that is less than about 200 μm. In other embodiments, the salt form of the therapeutic agent in the pharmaceutical compositions has a particle size distribution that is less than about 150 μm, less than about 100 μm, less than about 53 μm, less than about 38 μm, less than about 20 μm, less than about 10 μm, or less than about 5 μm. The salt form of the therapeutic agent in the pharmaceutical compositions of the invention can have a particle size distribution range for intranasal administration that is greater than about 5 μm, greater than about 10 μm, greater than about 15 μm, greater than about 20 greater than about 38 μm, less than about 53 μm, less than about 70 μm, greater than about 100 or greater than about 150 μm.

Additionally, the salt form of the therapeutic agent in the pharmaceutical compositions of the invention can have a particle size distribution range for pulmonary administration between about 1 μm and about 10 μm. In other embodiments for pulmonary administration, particle size distribution range is between about 1 μm and about 5 or about 2 μm and about 5 In other embodiments, the salt form of the therapeutic agent has a mean particle size of at least 1 μm, at least 2 μm, at least 3 μm, at least 4 μm, at least 5 μm, at least 10 at least 20 μm, at least 25 μm, at least 30 μm, at least 40 μm, at least 50 μm, at least 60 at least 70 μm, at least 80 μm, at least 90 μm, or at least 100 μm.

In some embodiments the disclosed cannabinoid compositions include one or more cannabinoids or pharmaceutically acceptable derivatives or salts thereof, a propellant, an alcohol, and a glycol and/or glycol ether. The alcohol may be a monohydric alcohol or a polyhydric alcohol, and is preferably a monohydric alcohol. Monohydric alcohol has a lower viscosity than a glycol or glycol ether. Accordingly, the composition is able to form droplets of a smaller diameter in comparison to compositions in which the monohydric alcohol is not present. The present inventors have surprisingly found that a specific ratio of monohydric alcohol to glycol or glycol ether results in a composition with a desired combination of both long term stability (for example the composition remains as a single phase for at least a week at a temperature of 2-40° C.) and small droplet size.

2. Pulmonary Compositions

One embodiment provides a formulation and method for treating SARS-COV-2 in the pulmonary system by inhalation or pulmonary administration. The diffusion characteristics of the particular drug formulation through the pulmonary tissues are chosen to obtain an efficacious concentration and an efficacious residence time in the tissue to be treated. Doses may be escalated or reduced or given more or less frequently to achieve selected blood levels. Additionally, the timing of administration of administration and amount of the formulation is preferably controlled to optimize the therapeutic effects of the administered formulation on the tissue to be treated and/or titrate to a specific blood level.

Diffusion through the pulmonary tissues can additionally be modified by various excipients that can be added to the formulation to slow or accelerate the absorption of drugs into the pulmonary tissues. For example, the drug may be combined with surfactants such as the phospholipids, dimyristoylphosphatidyl choline, and of administration dimyristoylphosphatidyl glycerol. The drugs may also be used in conjunction with bronchodilators that can relax the bronchial airways and allow easier entry of the antineoplastic drug to the lung. Albuterol is an example of the latter with many others known in the art. Further, the drug may be complexed with biocompatible polymers, micelle forming structures or cyclodextrins.

Particle size for the aerosolized drug used in the present examples was measured at about 1.0-5.0 μm with a GSD less than about 2.0 for deposition within the central and peripheral compartments of the lung. As noted elsewhere herein particle sizes are selected depending on the site of desired deposition of the drug particles within the respiratory tract.

Aerosols useful in the invention include aqueous vehicles such as water or saline with or without ethanol and may contain preservatives or antimicrobial agents such as benzalkonium chloride, paraben, and the like, and/or stabilizing agents such as polyethyleneglycol.

Powders useful in the invention include formulations of the neat drug or formulations of the drug combined with excipients or carriers such as mannitol, lactose, or other sugars. The powders used herein are effectively suspended in a carrier gas for administration. Alternatively, the powder may be dispersed in a chamber containing a gas or gas mixture which is then inhaled by the patient.

An agent of the present disclosure may be administered at once or may be divided into a number of multiple doses to be administered at intervals of time. For example, agents of the invention may be administered repeatedly, e.g., at least 2, 3, 4, 5, 6, 7, 8, or more times, or may be administered by continuous infusion. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that any concentration ranges set forth herein are exemplary only and are not intended to limit the scope or practice of the claimed compositions and methods.

In some therapeutic embodiments, an “effective amount” of an agent is an amount that results in a reduction of at least one pathological parameter. Thus, for example, in some aspects of the present disclosure, an effective amount is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not treated with the agent.

In some aspects, any of the PIV5-based constructs and methods described in WO 2013/112690 and WO 2013/112720 (which is hereby incorporated by reference herein in its entirety) may be used in the present invention.

As used herein, the term “subject” represents an organism, including, for example, a mammal. A mammal includes, but is not limited to, a human, a non-human primate, and other non-human vertebrates. A subject may be an “individual,” “patient,” or “host.” Non-human vertebrates include livestock animals (such as, but not limited to, a cow, a horse, a goat, and a pig), a domestic pet or companion animal, such as, but not limited to, a dog or a cat, and laboratory animals. Non-human subjects also include non-human primates as well as rodents, such as, but not limited to, a rat or a mouse. Non-human subjects also include, without limitation, poultry, horses, cows, pigs, goats, dogs, cats, guinea pigs, hamsters, mink, and rabbits.

As used herein “in vitro” is in cell culture and “in vivo” is within the body of a subject. As used herein, “isolated” refers to material that has been either removed from its natural environment (e.g., the natural environment if it is naturally occurring), produced using recombinant techniques, or chemically or enzymatically synthesized, and thus is altered “by the hand of man” from its natural state.

I. Booster Vaccines

The present disclosure provides for the administration of a booster PIV-5 vaccine for use in such a method for inducing in a human subject an immune response, wherein said subject has previously received a primary vaccination against SARS-COV-2.

The method of booster vaccination according to the disclosure comprises the step of administering the vaccine composition to the subject.

The immune response induced by the vaccine composition of the disclosure or by the method of the disclosure is preferably a humoral response, especially a response comprising the production of neutralizing antibodies against the COVID-19 virus, i.e. a neutralizing antibody response.

### F. Exemplary Embodiments

Exemplary Embodiments of the present disclosure include, but are not limited to, the following.

Embodiment 1: A viral expression vector comprising a parainfluenza virus 5 (PINS) genome having a heterologous nucleic acid sequence with at least 98% sequence identity to SEQ ID NOs: 61, 62, 63, 64, 65, 66, 67, 68, 69, 70, 71, 72, 73, 74, 75, 76, 77, 78, 79, or 80, wherein the viral expression vector expresses a heterologous polypeptide comprising a coronavirus spike (S) and/or nucleocapsid (N) proteins.

Embodiment 2: The viral expression vector of Embodiment 1, wherein the coronavirus S protein is a coronavirus S protein of the severe acute respiratory syndrome coronavirus 2 (SARS-CoV-2), a variant of interest or a variant of concern of SARS-CoV-2 and the coronavirus N protein is the coronavirus N protein of SARS-CoV-2, a variant of interest or a variant of concern of SARS-CoV-2.

Embodiment 3: The viral expression vector of Embodiment 1, wherein the coronavirus S protein is the coronavirus S protein of a SARS-CoV-2 beta variant, a SARS-CoV-2 gamma variant, a SARS-CoV-2 delta variant, or a SARS-CoV-2 omicron variant and the coronavirus N protein is the coronavirus N protein of a SARS-CoV-2 beta variant, a SARS-CoV-2 gamma variant, a SARS-CoV-2 delta variant, or a SARS-CoV-2 omicron variant.

Embodiment 4: The viral expression vector of Embodiment 1, wherein the coronavirus S protein comprises the coronavirus S protein of SARS-CoV-2 and wherein the cytoplasmic tail of the coronavirus S protein has been replaced with the cytoplasmic tail of the fusion (F) protein of PIV5.

Embodiment 5: The viral expression vector of Embodiment 1, wherein the coronavirus S protein of a variant of SARS-CoV-2 has been inserted between the PIV5 small hydrophobic (SH) and hemagglutinin (HN) genes and the coronavirus N protein of a variant of SARS-CoV-2 has been inserted between the PIV5 HN and polymerase (L) genes.

Embodiment 6: The viral expression vector of any one of Embodiments 1, wherein the coronavirus S protein of a variant of SARS-CoV-2 has been inserted between the PIV5 HN and polymerase (L) genes.

Embodiment 7: The viral expression vector of Embodiment 1, wherein the coronavirus S protein is a S protein from a variant of interest or a variant of concern of SARS-CoV-2 and the coronavirus N protein is a N protein from different variant of interest or a variant of concern of SARS-CoV-2.

Embodiment 8: The viral expression vector of Embodiment 1, wherein the PIV5 small hydrophobic (SH) gene has been replaced with the coronavirus N protein of SARS-CoV-2.

Embodiment 9: The viral expression vector of Embodiment 1, wherein the PIV5 genome further comprises one or more mutations comprising a mutation of the V/P gene, a mutation of the shared N-terminus of the V and P proteins, a mutation of residues 26, 32, 33, 50, 102, and/or 157 of the shared N-terminus of the V and P proteins, a mutation lacking the C-terminus of the V protein, a mutation lacking the small hydrophobic (SH) protein, a mutation of the fusion (F) protein, a mutation of the phosphoprotein (P), a mutation of the large RNA polymerase (L) protein, a mutation incorporating residues from canine parainfluenza virus, a mutation inducing apoptosis, or a combination thereof.

Embodiment 10: The viral expression vector of Embodiment 9, wherein the one or more mutations comprise PIV5VΔC, PIV5ΔSH, PIV5-P-S308G, or a combination thereof.

Embodiment 11: The viral expression vector of Embodiment 1, wherein the heterologous polypeptide comprises a CPI V/P gene that contains mutations at amino acid residue S157 or S308, or the combination thereof, wherein serine (S) is substituted with an amino acid residue selected from a group consisting of alanine (A), asparagine (B), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), selenocysteine (U), valine (V), tryptophan (W), tyrosine (Y), and glutamine (Z).

Embodiment 12: The viral expression vector of Embodiment 11, wherein the amino acid substitution at amino acid residue S157 comprises a substitution of serine (S) to phenylalanine (F) and the amino acid substitution at amino acid residue S308 comprises a substitution of serine (S) to alanine (A) or Glycine (G).

Embodiment 13: The viral expression vector of Embodiment 1, wherein a viral particle comprises the viral expression vector.

Embodiment 14: A composition comprising the viral expression vector of Embodiment 1 or the viral particle of Embodiment 13 or a combination thereof.

Embodiment 15: The composition of Embodiment 14, wherein a heterologous coronavirus spike (S) and nucleocapsid (N) proteins are expressed in a cell by contacting the cell with the composition.

Embodiment 16: A method of inducing an immune response in a subject having coronavirus disease 2019 (COVID-19) to coronavirus spike (S) and nucleocapsid (N) proteins, the method comprising administering the composition of Embodiment 14 to the subject, wherein the immune response comprises a humoral immune response and/or a cellular immune response.

Embodiment 17: The method of Embodiment 16, wherein the subject is vaccinated against COVID-19), the method comprising administering the composition to the subject.

Embodiment 18: The method of Embodiment 16, wherein the composition is administered intranasally, intramuscularly, topically, or orally.

Embodiment 19: A method of inducing in a subject an immune response comprising administering a PIV-5 booster vaccine composition comprising a viral expression vector or a viral particle having a PIV5 genome comprising a heterologous nucleic acid sequence with at least 98% sequence identity to SEQ ID NOs: 61, 62, 63, 64, 65, 66, 67, 68, 69, 70, 71, 72, 73, 74, 75, 76, 77, 78, 79, or 80, wherein said subject has previously received a primary vaccination against SARS-COV-2.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

For any method disclosed herein that includes discrete steps, the steps may be conducted in any feasible order. And, as appropriate, any combination of two or more steps may be conducted simultaneously.

The description exemplifies illustrative embodiments. In several places throughout the application, guidance is provided through lists of examples, which examples can be used in various combinations. In each instance, the recited list serves only as a representative group and should not be interpreted as an exclusive list.

All headings are for the convenience of the reader and should not be used to limit the meaning of the text that follows the heading, unless so specified.

The present invention is illustrated by the following examples. It is to be understood that the particular examples, materials, amounts, and procedures are to be interpreted broadly in accordance with the scope and spirit of the invention as set forth herein.

### EXAMPLES

### Example 1: Vaccine Efficacy Study in a Syrian Hamster SARS-CoV-2 Challenge Model

Materials:

The SARS-CoV-2 vaccine candidates generated are described herein.

CVL34 for CVXGA2: The N gene of SARS-CoV-2 Wuhan strain was amplified by PCR, inserted into CPI containing the S gene of SARS-CoV-2 Wuhan strain by Gibson assembly. The recombinant virus was rescued on 293-T serum free cells, which contains the S gene of SARS-CoV-2 Wuhan strain between SH and HN, the N gene of SARS-CoV-2 Wuhan strain between HN and L of CPI.

CVL44 for CVXGA3: The S gene of SARS-CoV-2 Beta variant was amplified by PCR, inserted into CPI backbone by Gibson assembly. The recombinant virus was rescued on 293-T serum free cells, which contains the S gene of SARS-CoV-2 Beta variant between SH and HN of CPI.

CVL48 for CVXGA4: The S gene of SARS-CoV-2 Alpha variant was digested from a plasmid containing the gene generated from Genscript, inserted into CPI backbone by Gibson assembly. The recombinant virus was rescued on 293-T serum free cells, which contains the S gene of SARS-CoV-2 Alpha variant between SH and HN of CPI.

CVL49 for CVXGA5: The S gene of SARS-CoV-2 Gamma variant was digested from a plasmid containing the gene generated from Genscript, inserted into CPI backbone by Gibson assembly. The recombinant virus was rescued on 293-T serum free cells, which contains the S gene of SARS-CoV-2 Gamma variant between SH and HN of CPI.

CVL50 for CVXGA6: The S gene of SARS-CoV-2 Epsilon variant was amplified by PCR, inserted into CPI backbone by Gibson assembly. The recombinant virus was rescued on Vero serum free cells, which contains the S gene of SARS-CoV-2 Epsilon variant between SH and HN of CPI.

CVL52 for CVXGA7: The N gene of SARS-CoV-2 Wuhan strain was amplified by PCR, inserted into CVL44 backbone by Gibson assembly. The recombinant plasmid contains the N gene of SARS-CoV-2 Wuhan strain and the S gene of SARS-CoV-2 Beta variant between F and HN, and lacking the CPI SH gene. The plasmid construct is being generated.

CVL53 for CVXGA8: The N gene of SARS-CoV-2 Wuhan strain was amplified by PCR, inserted into CPI backbone by Gibson assembly. The recombinant virus was rescued on 293-T serum free cells, which contains the N gene of SARS-CoV-2 Wuhan strain between F and HN, and the SH gene deletion of CPI.

CVL54 for CVXGA9: The S gene of SARS-CoV-2 Beta variant was amplified by PCR, inserted into CVL53 backbone by Gibson assembly. The recombinant plasmid contains the N gene of SARS-CoV-2 Wuhan strain between F and HN, the S gene of SARS-CoV-2 Beta variant between HN and L, and the SH gene deletion of the CPI backbone. The plasmid construct is being generated.

CVL55 for CVXGA10: CPI-V/P gene containing the S157F and S308A mutations (PLK mutations) was amplified by PCR, inserted into CVL44 backbone by Gibson assembly. The recombinant virus was rescued on 293-T serum free cells, which contains the S gene of SARS-CoV-2 Beta variant between SH and HN, and the S157F and S308A mutations in CPI-V/P gene.

CVL58 for CVXGA11: CPI with the SH gene deletion (CPIΔSH) was obtained by PCR amplification and Gibson assembly in CVL44 backbone. The recombinant plasmid containing the S gene of SARS-CoV-2 Beta variant between F and HN, and the CPI SH gene deletion, was obtained. Virus rescue is being rescued.

CVL59 for CVXGA12: CPI lacking SH gene (CPIΔSH) was obtained by PCR amplification and Gibson assembly in CVL55 backbone. The recombinant plasmid has been obtained, which contains the S gene of SARS-CoV-2 Beta variant between F and HN, CPI SH gene deletion, and S157F and S308A mutations in CPI-V/P gene.

CVL60 for CVXGA13: The S gene of SARS-CoV-2 Delta variant was digested from a plasmid containing the gene generated from Genscript, then inserted into CPI backbone by Gibson assembly. The recombinant CVGA13 virus was rescued in Vero serum free Vero cells, which contains the S gene of SARS-CoV-2 Delta variant between SH and HN.

CVL64 for CVXGA26: The S gene of SARS-CoV-2 Lambda variant was digested from a plasmid containing the gene generated in-house, then inserted into CPI backbone by Gibson assembly. The recombinant plasmid contains the S gene of SARS-CoV-2 Lambda variant between SH and HN. The plasmid construct is being generated.

CVL65 for CVXGA27: The S gene of SARS-CoV-2 Delta variant was amplified by PCR and inserted into CVL54 backbone by Gibson assembly. The recombinant virus will contain N gene of SARS-CoV-2 Wuhan strain between F and HN, S gene of SARS-CoV-2 Delta variant between HN and L, and lacking SH gene of CPI backbone. The plasmid DNA construct will be generated.

CVL67 for CVXGA28: The S gene of SARS-CoV-2 Mu variant will be digested from a plasmid containing the gene generated in-house and inserted into CPI backbone by Gibson assembly. The recombinant virus will contain S gene of SARS-CoV-2 Mu variant between SH and HN. The primers have been ordered for making the plasmid construct.

CVL80 for CVXGA14: The S gene of SARS-CoV-2 Omicron BA.1 variant was generated by Genscript, then inserted into CPI backbone by Gibson assembly. The recombinant plasmid contains the S gene of SARS-CoV-2 Omicron BA.1 variant between SH and HN. The recombinant virus is obtained.

CVL86 for CVXGA15: The 5′ end of S gene of SARS-CoV-2 Omicron BA.2 variant was generated by Genscript, 3′ end of S gene was amplified by mutagenesis of the PCR reactions and inserted into CPI backbone by Gibson assembly. The recombinant plasmid contains the S gene of SARS-CoV-2 Omicron BA.2 variant between the SH and HN genes. The recombinant virus rescue is ongoing.

CVL108 for CVXGA20: The S gene of SARS-CoV-2 Omicron BA.5 variant was amplified and inserted into CVL86 backbone to replace the S gene of BA.2 variant by Gibson assembly. The recombinant plasmid contains the S gene of SARS-CoV-2 Omicron BA.5 variant between the SH and HN genes of PIV5. The recombinant virus is obtained.

CVL111 for CVXGA23: The 5′ end of S gene of SARS-CoV-2 Omicron BA.2.75 variant was generated by Genscript, and 3′ end of S gene was amplified from CVL86 plasmid and inserted into CVL6 backbone to replace the S gene of Wuhan strain by Gibson assembly. The recombinant plasmid will contain S gene of SARS-CoV-2 Omicron BA.2.75 variant between the SH and HN genes of PIV5.

CVL113 for CVXGA25: The PIV5 F and HN genes was deleted from CVL54 backbone by Gibson assembly. The recombinant plasmid will contain CPI backbone without F and HN genes, and S gene of SARS-CoV-2 Wuhan strain between HN and L genes of PIV5. The recombinant plasmid construction is ongoing. The recombinant virus will only contain the glycoprotein from the SARS-COV-2 virus. This virus can serve either as the vaccine strain and/or the virus for microneutralization assay to measure S-specific antibody response.

The primers used in cDNA cloning of the CPI-S vaccine candidates are listed in Table 1.

TABLE 1
Primer sequences used in constructing CPI
based COVID-19 S vaccine candidates

| | | | SEQ |
|---|---|---|---|
| Con- | | | ID |
| struct | Primer | Sequence | NO: |
| CVL34 | oCVL101 | ACATTAATGGCATCAATCAGTTCTATT | 1 |
| | | TCTCAATT | |
| | oCVL106 | AGATTAGAACATGATGATTGGGTACAT | 2 |
| | | TCT | |
| CVL44 | oCVL119 | CGGTGCCAACAACACAATCCACAATCT | 3 |
| | | ACA | |
| | oCVL120 | GCTATTGTTAGAGTAGGCCACGCTGTT | 4 |
| | | CTC | |
| | oCVL121 | GAGAACAGCGTGGCCTACTCTAACAAT | 5 |
| | | AGC | |
| | oCVL122 | ATATGATGTGAGTAGGAAGTATGGCAG | 6 |
| | | GAG | |
| CVL50 | oCVL119 | CGGTGCCAACAACACAATCCACAATCT | 7 |
| | | ACA | |
| | oCVL127 | GTGAATTGTACCGAGGTGCCCGTGGCT | 8 |
| | | ATC | |
| | oCVL128 | GATAGCCACGGGCACCTCGGTACAATT | 9 |
| | | CAC | |
| | oCVL122 | ATATGATGTGAGTAGGAAGTATGGCAG | 10 |
| | | GAG | |
| CVL52 | oCVL17 | CAAGATGCACCTTCTCTCCAGTG | 11 |
| | oCVL132 | GTTCTGTGGTCCGTTATCTGACATGGT | 12 |
| | | GGCGCGCGATTTGAGCTTTCCGAGACG | |
| | | GTAGC | |
| | oCVL133 | GCGCGCCACCATGTCAGATAACGGACC | 13 |
| | | ACAGAAC | |
| | oCVL134 | TCAAGCCTGGGTGGAGTCGGCACTG | 14 |
| | oCVL135 | CAGTGCCGACTCCACCCAGGCTTGACG | 15 |
| | | TACGACCTGCTATAGGCTATCCACTGC | |
| | | ATC | |
| | oCVL124 | CAGGCCGTTGAAATTGAAGT | 16 |
| CVL53 | oCVL17 | CAAGATGCACCTTCTCTCCAGTG | 17 |
| | oCVL132 | GTTCTGTGGTCCGTTATCTGACATGGT | 18 |
| | | GGCGCGCGATTTGAGCTTTCCGAGACG | |
| | | GTAGC | |
| | oCVL133 | GCGCGCCACCATGTCAGATAACGGACC | 19 |
| | | ACAGAAC | |
| | oCVL136 | GATATGATGTGAGTAGGAAGTATGGCA | 20 |
| | | GGAGGCTAGCTCAAGCCTGGGTGGAGT | |
| | | CGGC | |
| CVL54 | oCVL101 | ACATTAATGGCATCAATCAGTTCTATT | 21 |
| | | TCTCAATT | |
| | oCVL137 | TTAGGATAGTGTCACCTGACGGATAAA | 22 |
| | | TGG | |
| | oCVL138 | CCATTTATCCGTCAGGTGACACTATCC | 23 |
| | | TAAACCTGCTATAGGCTATCCACTGC | |
| | oCVL139 | TCATTTATGATAAACAAAATTCTCC | 24 |
| | oCVL140 | GGAGAATTTTGTTTATCATAAATGACG | 25 |
| | | TACGAGGCAGAAGCCTCCAGGTCTGAC | |
| | | CCAGCC | |
| | oCVL106 | AGATTAGAACATGATGATTGGGTACAT | 26 |
| | | TCT | |
| CVL55 | oCVL141 | GCAAGATCAGAGTGAGGAAGG | 27 |
| | oCVL142 | AATTGGTTGCGATAGGATTCTCTCTG | 28 |
| | oCVL143 | CCCCATCGATTTTAAGAGGGGCAGG | 29 |
| | oCVL144 | GGCATCATTGAATGATTCTGGCACAAC | 30 |
| | oCVL145 | TTCTTGACTCAATCTGAAGATGTAA | 31 |
| | oCVL146 | GCTGTAGGATCACAGCAGCA | 32 |
| CVL58 | oCVL17 | CAAGATGCACCTTCTCTCCAGTG | 33 |
| and | oCVL159 | CGTACGTTAGCGCGCGATTTGAGCTTT | 34 |
| 59 | | CCGAGACG | |
| | oCVL160 | TCTCGGAAAGCTCAAATCGCGCGCTAA | 35 |
| | | CGTACGCACCTGCTATAGGCTATCCAC | |
| | | TGCATC | |
| | oCVL161 | AAACTGCAAGGGGTGATGTC | 36 |
| CVL65 | oCVL119 | CGGTGCCAACAACACAATCCACAATCT | 37 |
| | | ACA | |
| | oCVL262 | GGCTGGGTCAGACCTGGAGGCTTCTGC | 38 |
| | | CTCGTACGAATTCATTTATGATAAACA | |
| | | AAATTC | |
| CVL86 | oCVL422 | AACACAATCCACAATCTACAGTC | 39 |
| | oCVL423 | GGTCAGGCCGTTGAAGTTGAAGTTCA | 40 |
| CVL108 | oCVL422 | AACACAATCCACAATCTACAGTC | 41 |
| | oCVL611 | GATGGCGTGGAACCAGGTCACGTTG | 42 |
| | oCVL612 | CAACGTGACCTGGTTCCACGCCATCAG | 43 |
| | | CGGCACCAATGGCACAAAGCG | |
| | oCVL613 | CGGCCTGGTAGATTTCTGTAGAGATGT | 44 |
| | | CCCTCTCGAAGGGCTTCAGATTGCTCT | |
| | | TTCTAAACAGCCGGTATCTATAATTGT | |
| | | AGTTGCCGC | |
| | oCVL614 | TACAGAAATCTACCAGGCCGGCAATAA | 45 |
| | | GCCTTGCAATGGCGTGGCCGGCGTGAA | |
| | | CTGTTATTTCCCACTCCAGTCCTACGG | |
| | | CTTCCGCCC | |
| | oCVL122 | ATATGATGTGAGTAGGAAGTATGGCAG | 46 |
| | | GAG | |
| CVL111 | oCVL422 | AACACAATCCACAATCTACAGTC | 47 |
| | oCVL618 | TCCCTTCCTGGACGTGTACTATCAC | 48 |
| | oCVL619 | CATATGTGGGGCGGAAGCCGTAGGA | 49 |
| | oCVL620 | GTGATAGTACACGTCCAGGAAGGGA | 50 |
| | oCVL621 | CAGTCCTACGGCTTCCGCCCCAC | 51 |
| | oCVL122 | ATATGATGTGAGTAGGAAGTATGGCAG | 52 |
| | | GAG | |
| CVL113 | oCVL5 | ACAGTGGATTCCATAAGGTTCCTGC | 53 |
| | oCVL6 | GGAGAAGCTCAGATCAGTGGGATC | 54 |
| | oCVL622 | AGCTCATTCCAGCTCCGTCAGGTT | 55 |
| | oCVL624 | AACCTGACGGAGCTGGAATGAGCTAG | 56 |
| | | CCCTGCTATAGGCTATCCACTGCATCA | |
| | oCVL625 | CGATAGGCAGGTCCACCAG | 57 |
| | oCVL626 | GGTCAGACCTGGAGGCTTCTGCCTCGT | 58 |
| | | ACGTCATCATTTATGATAAACAAAATT | |
| | | CTCC | |
| | oCVL119 | CGGTGCCAACAACACAATCCACAATCT | 59 |
| | | ACA | |
| | oCVL166 | ACCAGGGGGAAAACGAAGTGG | 60 |

Note:
The inserts for CVL48, 49, 60, 64, 67 and 80 are digested from plasmids, not PCR.

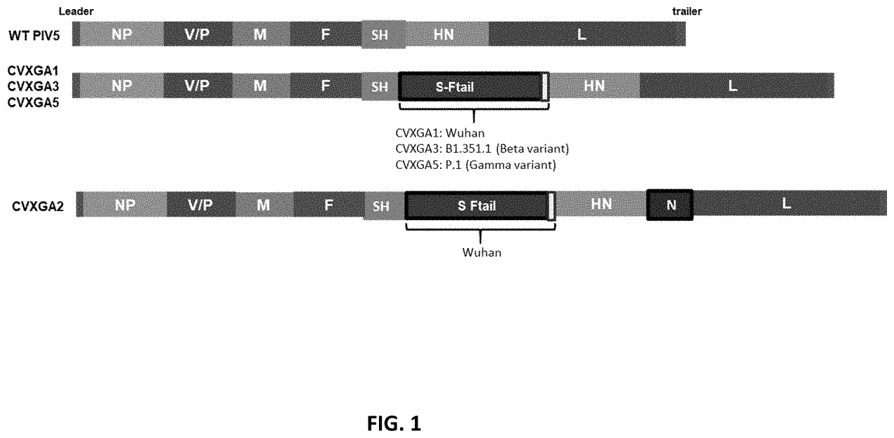

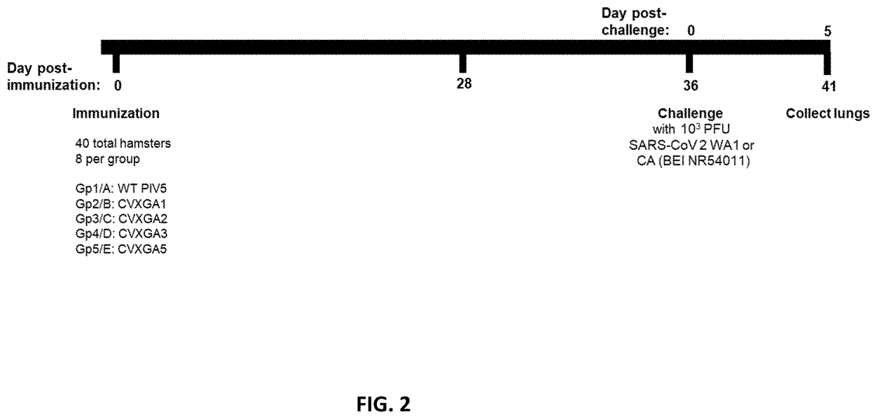

Five candidate vaccines based on the PIV5 CPI vector were evaluated in this study (Table 2). CVXGA1, 2, 3, and 5 have a SARS-CoV-S gene inserted between the PIV5 small hydrophobic (SH) and hemagglutinin (HN) genes. CVXGA2 has an additional insert, the SARS-CoV-2 nucleoprotein (N), inserted between the PIV5 HN and polymerase (L) genes (FIG. 1).

TABLE 2
Vaccine strains used in the hamster study

| | | Vaccine dose |
|---|---|---|
| Virus | Insert & SARS-CoV-2 strain | (PFU/hamster |
| WT PIV5 | None | 9.7 × 10⁴ |
| CVXGA1 | S from Wuhan strain | 9.8 × 10⁴ |
| CVXGA2 | S and N from Wuhan strain | 8.7 × 10⁴ |
| CVXGA3 | S from Beta variant | 8.8 × 10⁴ |
| CVXGA5 | S from Gamma variant | 4.8 × 10² |

Study Outline.

Forty male, 5-7 week-old Syrian hamsters were used in this study (N=8 per vaccine group). The hamsters were intranasally immunized with 100 μL of WT PIV5 CPI virus, CVXGA1, CVXGA2, and CVXGA3 of approximately 1×105 plaque-forming units (PFU). For CVXGA5, hamsters received 100 uL of approximately 5×102 PFU (Table 2). At 36 days post-immunization (dpi), four hamsters from each group were challenged with 103 PFU of SARS-CoV-2 Wuhan strain (WA1), and the remaining four hamsters from each group were challenged with 103 PFU SARS-CoV-2 alpha variant (CA; BEI NR54011). Following challenge infection, the hamster weights were monitored for 5 days, the hamster lungs were harvested, and SARS-CoV-2 viral burden was quantified via plaque assay and real-time quantitative reverse transcription polymerase chain reaction (qRT-PCR).

Determination of infectious SARS-CoV-2 viral burden in hamster lungs post-challenge. Hamster lung homogenates were serially diluted in Dulbecco's modified Eagle medium (DMEM)+2% fetal bovine serum (FBS)+1% antibiotic/antimycotic and added to 12-well plates of Vero E6 cells. At 1 hour post-infection, the inoculum was removed, and a methylcellulose overlay (500 mL Opti-MEM+0.8% methylcellulose+2% FBS+1% antibiotic/antimycotic) was added to the wells. The plates were incubated for 3 days, the overlay was removed, and the cells were fixed with 40% acetone/60% methanol. The cells were stained with crystal violet, the number of plaques were counted and viral titers are expressed as log 10 PFU/mL.

Quantification of SARS-CoV-2 RNA load in hamster lungs post-challenge by qRT-PCR. For the standard curves, SARS CoV-2 challenge virus at concentrations of 2×107 PFU/mL and 4×108 PFU/mL for WA1 and CA1 respectively, was inactivated by 1:10 diluted Trizol reagent (ThermoFisher Scientific) with the genetic material preserved. Hamster lung homogenate was also treated with 1:10 diluted Trizol. Using a Qiagen Viral RNA extraction kit, RNA was extracted from 140 μL inactivated virus and lung homogenate and eluted in 15 buffer. WA1 and CA1 RNA was serial-diluted 1:10 in sterile water to serve as the standard curves. For the qRT-PCR reactions, 5 μL eluted RNA was mixed with 5 μL Thermofisher TaqPath 1-Step qPCR Master Mix (cat #A15300), 1.5 μL 2019-nCoV CDC EUA Authorized qPCR Probe Assay primer/probe mix, IDT (cat #10006770), and 8.5 μL water. qRT-PCR thermocycler settings were used as described on “CDC 2019-Novel Coronavirus (2019-nCoV) Real-Time RT-PCR Diagnostic Panel . . . Instructions for Use” page 26. To generate the standard curves, the viral titer was plotted on the x-axis and the cycle threshold (CT) value was plotted on the y-axis.

Results.

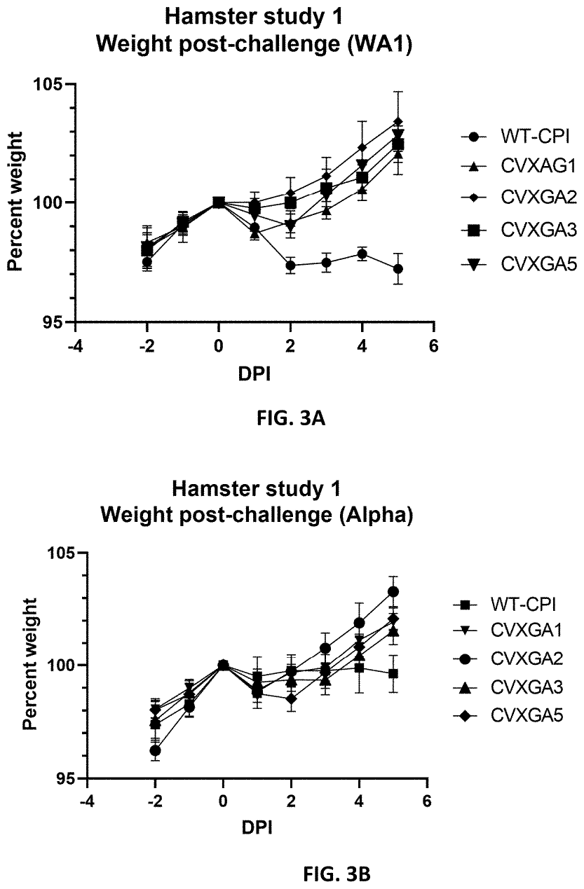

Hamster weight changes post-challenge infection. Following challenge with either WA1 or CA1/alpha SARS-CoV-2 variants, the hamster weights were monitored daily. For hamsters challenged with the WA1 variant, those immunized with CVXGA1, CVXGA2, CVXGA3, or CVXGA5 had minimal weight loss and resumed weight gaining within 5 dpc. However, the hamsters immunized with WT PIV5 CPI strain had weight loss and did not regain weight by the end of the study at 5 dpc (FIG. 3A). For the hamsters challenged with CA1/alpha, hamsters immunized with CVXGA1, CVXGA2, CVXGA3, or CVXGA5 had transient weight loss and resumed gaining weight by 3 dpc and had all surpassed their starting weight by 5 dpc. In contrast, the hamsters immunized with WT PIV5 maintained their starting weight (FIG. 3B).

CVXGA2 encoding both the S and N protein from SARS-CoV-2 Wuhan strain had the best weight gain after the viral challenge, indicating additonal protection offered by the inserted N gene in the vaccine candidate. In addition, the vaccine dose of CVXGA5 was approx. 100-fold lower than the other vaccine viruses, yet it offered protection as good as CVXGA1 and CVXGA3.

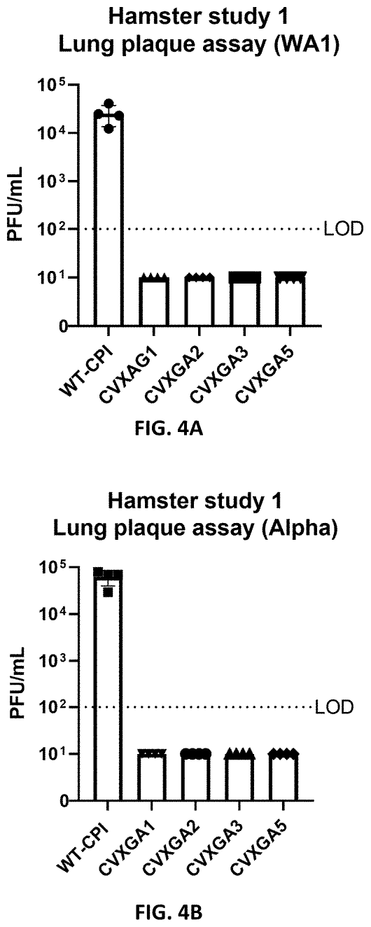

SARS-CoV-2 challenge virus titer in the lung. At 5 dpc, SARS-CoV-2 viral burden in the hamster lungs was quantified. Hamsters immunized with WT PIV5 CPI had an average titer of 2.5×104 and 6.2×104 PFU/mL following challenge with WA1 and CA1/alpha, respectively. In contrast, none of the CVXGA-immunized hamsters had detectable infectious virus in their lungs (FIGS. 4A-4B). This data demonstrated that the CPI-vectored SARS-CoV-2 vaccine candidates, including CVXGA5 with 100-fold lower dose, offered complete protection in the lower respiratory tract of hamster against homologous and heterologous virus challenge infections.

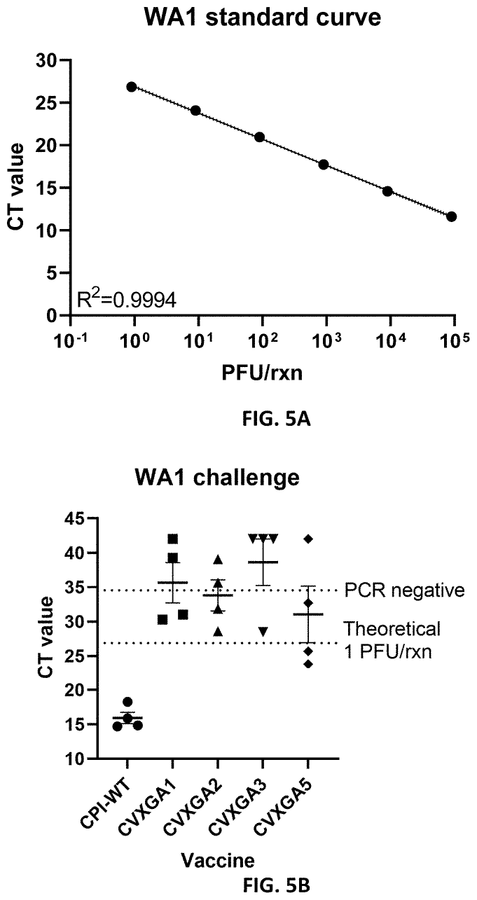

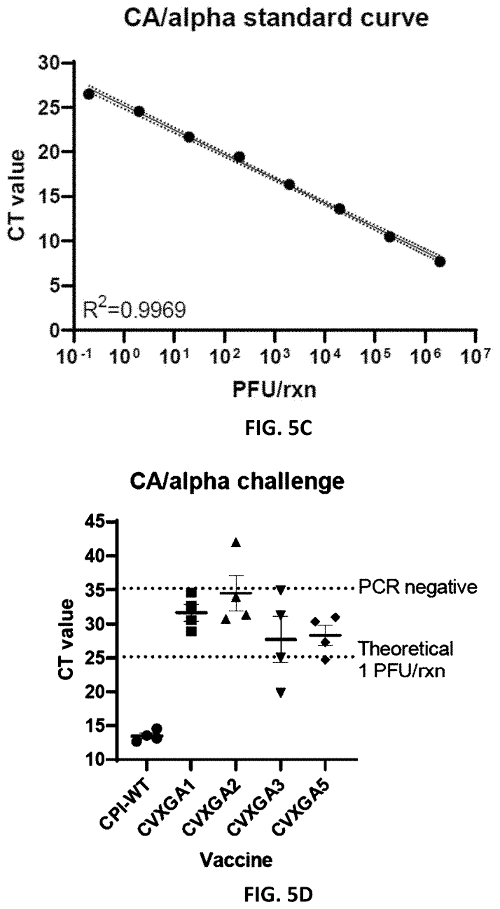

Quantitation of SARS-CoV-2 RNA in the vaccinated hamster lungs following challenge infection. SARS-CoV2 challenge virus RNA load in the hamster lungs were quantitated by qRT-PCR. Note that a lower cycle threshold (CT) value corresponds to a higher viral titer. Hamsters immunized with WT PIV5 CPI and challenged with WA1, had CT values between 14.7 and 18.2, which equates to 8.9×103 and 6.5×102 PFU/rxn (FIGS. 5A-5B). Hamsters immunized with WT PIV5 CPI and challenged with CA1/alpha strain, had CT values between 12.7 and 14.5, which equates to 3.8×104 and 8.3×103 PFU/rxn (FIGS. 5B-5D). For both challenge SARS-CoV2 variants, hamsters immunized with CVXGA1 or CVXGA2 had CT values indicative of less than one infectious viral particle per rxn (FIGS. 5A-5D). Three of four hamsters immunized with CVXGA3 and challenged with WA1 were PCR-negative, and the fourth hamster had a CT value corresponding to less than one infectious virus particle per reaction (FIGS. 5A-5B). For the hamsters immunized with CVXGA3 and challenged with CA1/alpha, one hamster was PCR-negative and two hamsters had CT values indicative of less than one infectious particle per reaction. The remaining hamster had CT value 19.78, which corresponds to approximately 100 PFU/rxn (FIGS. 5B-5D). For the CVXGA5-immunized hamsters, one WA1-challenged hamster was PCR-negative, while one WA1-challenged and three CA1/alpha-challenged hamsters had CT values indicative of less than one infectious particle per mL. For the remaining CVXGA5-immunized hamsters, two challenged with WA1 had CT values correlating to less than 10 PFU/rxn and one challenged with CA1/alpha had a CT value corresponding to approximately 1.5 PFU/rxn (FIGS. 5A-5D). Compared to the PIV5 CPI immunized animals, CVXGA1, 2, 3 and 5 immunized hamsters had greatly reduced challenge virus burden not only to the homologous WA1 strain but also to the heterologous CA/alpha strain, which were either undetectable or at very low levels.

In order to protect against SARS-CoV-2 variants, recombinant PIV5 CPI-vectored candidate vaccines expressing the S protein alone or with S and N from SARS-CoV-2 Wuhan strain, beta, and gamma variants have been developed. All four CVXGA candidate vaccines offered significant protection from weight loss not only from homologous but also heterologous SARS-CoV-2 challenge virus infection. Furthermore, no infectious SARS-CoV-2 live virus was detected in the lungs of CVXGA-immunized hamster 5 dpc, which was confirmed by the low/no viral RNA burden by qRT-PCR assay. These results indicate that PIV5 CPI vectored CVXGA candidate SARS-CoV-2 vaccines are effective in providing protection against SARS-CoV-2 variants.

### Example 2: Protective Efficacy of CVXGA1 and CVXGA3 in AGM

Materials and methods.

Two candidate SARS-CoV-2 vaccine viruses based on the PIV5 CPI vector were evaluated in this study. CVXGA 1 and 3 have the SARS-CoV-S gene from variants Wuhan (WA1) and California/alpha (CA1/alpha), respectively, inserted between the PIV5 small hydrophobic (SH) and hemagglutinin (HN) genes. BLB-201 has the fusion (F) gene from respiratory syncytial virus (RSV) in the same location as the control (FIG. 1).

Study Outline.

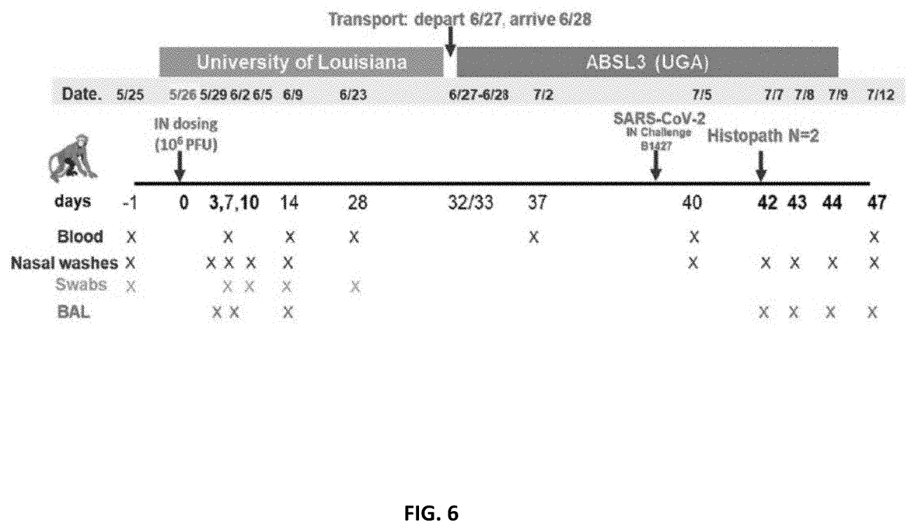

Twelve AGMs were used in this study with 4 per vaccine group. The AGMs were intranasally immunized with 106 PFU/AGM of vaccine virus. Serum was collected prior to dosing (Day −1) and 28 days post-immunization (dpi) to quantify the anti-S IgG antibody titer via enzyme-linked immunosorbent assay (ELISA). Blood was collected at −1, 7, 14, and 28 dpi to quantify the CoV-S-specific cellular response. At 40 dpi, the mice were challenged with 1.5×106 PFU SARS-CoV-2 CA1/alpha variant. At 2, 4, and 7 days post-challenge (dpc), AGM nasal washes (NW) and bronchoalveolar lavage (BAL) were collected, and SARS-CoV-2 viral burden was quantified via plaque assay and qRT-PCR (FIG. 6 and Table 3).

TABLE 3
Efficacy of CVXGA1 and CVXGA3 in African green monkeys

| Group | Animal | | | |
|---|---|---|---|---|
| (N = 4) | ID | Virus | Gene insert | Lot# and titer |
| Group 4 | 9867 | CVXGA1 | CPI-S | 5/1/17 SP |
| | 9845 | | (Wuhan) | 1.5 × 10⁷ pfu/ml |
| | 58169 | | | |
| | 9829 | | | |
| Group 6 | 9669 | BLB-201 | CPI-RSV-F | 210322MCBCHD |
| | 9839 | | | 2 × 10⁷ pfu/ml |
| | 9854 | | | |
| | 9861 | | | |
| Group 7 | 9866 | CVXGA3 | CPI-S- | 210427ZL |
| | 9851 | | B.1.135.1 | 2.1 × 10⁷ pfu/ml |
| | 9814 | | | |
| | 9828 | | | |

ELISA to quantify anti-SARS-Cov-2-S IgG antibodies. For the enzyme-linked immunosorbent assay (ELISA), Immulon 2HB 96-well microtiter plates were coated with 100 μL/well purified SARS-CoV-2 S protein at 1 μg/mL. Serum samples were diluted 3-fold in KPL Wash Solution (SeraCare Life Sciences, Inc., Milford, Mass.) supplemented with 0.5% bovine serum albumin (BSA) and 5% nonfat milk. Serum samples were incubated at 100 μL/well for 1 h. After washing with KPL Wash Solution three times, the plates were incubated for 1 h with a 1:1000 dilution of horseradish peroxidase-labelled goat anti-monkey IgG antibody (Southern Biotech, Birmingham, Ala.) in KPL Wash Solution supplemented with 0.5% BSA and 5% nonfat milk. After the plates were washed three times, antibody-antigen binding was realized using KPL SureBlue Reserve TMB Microwell Peroxidase Substrate (SeraCare Life Sciences, Inc.) and a Glomax 96 microplate luminometer (Promega) set at OD₄₅₀. An antibody titer was calculated as the highest serum dilution at which the OD₄₅₀ was greater than two standard deviations above the mean OD₄₅₀ of naïve serum.

ELISPOT to quantify SARS-CoV-2-S-specific cellular response. SARS-CoV-2-specific peripheral blood mononuclear cells (PBMCs) in blood were quantified by ELISpot assay at days −1, 7, 14, and 28 dpi. The number of spots was reported as the number of IFN-γ spots per 2×105 PBMCs.

Plaque assays to determine infectious SARS-CoV-2 viral burden in AGM NW and BAL post-challenge. NW and BAL were serially diluted in Dulbecco's modified Eagle medium (DMEM)+2% fetal bovine serum (FBS)+1% antibiotic/antimycotic and added to 12-well plates of Vero E6 cells. At 1 hour post-infection, the inoculum was removed, and a methylcellulose overlay (500 mL Opti-MEM+0.8% methylcellulose+2% FBS+1% antibiotic/antimycotic) was added to the wells. The plates were incubated for 3 days, the overlay was removed, and the cells were fixed with 40% acetone/60% methanol. The cells were stained with crystal violet, the number of plaques were counted, and viral titers are expressed as log 10 PFU/mL.

Quantification of SARS-CoV-2 RNA in AGM NW and BAL by qRT-PCR. AGM NW and BAL samples were inactivated by 1:10 diluted Trizol reagent (ThermoFisher Scientific) with the genetic material preserved. Using a Qiagen Viral RNA extraction kit, RNA was extracted from 140 μL inactivated sample and eluted in 15 μL buffer. For the qRT-PCR reactions, 5 μL eluted RNA was mixed with 5 μL Thermofisher TaqPath 1-Step qPCR Master Mix (cat #A15300), 1.5 μL 2019-nCoV CDC EUA Authorized qPCR Probe Assay primer/probe mix, IDT (cat #10006770), and 8.5 μL water. qRT-PCR thermocycler settings were used as described on “CDC 2019-Novel Coronavirus (2019-nCoV) Real-Time RT-PCR Diagnostic Panel . . . Instructions for Use” page 26.

Results.

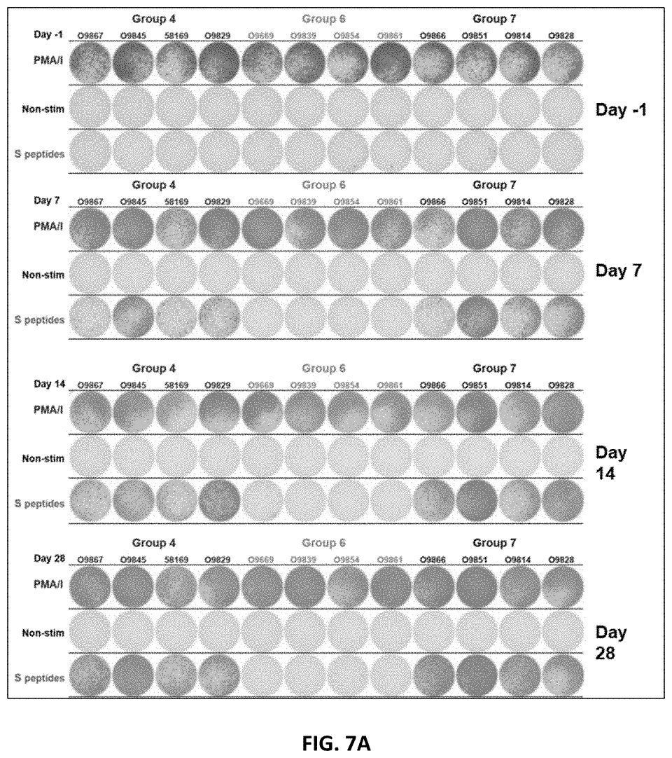

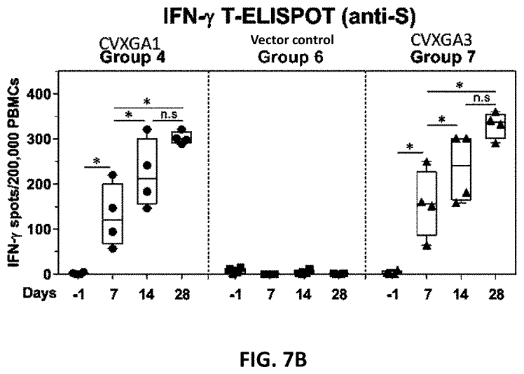

SARS-CoV-2-S-specific cellular response. Following immunization, blood was collected at days −1, 7, 14, and 28 to quantify the SARS-CoV-2-S-specific cellular response. For all groups, PBMCs collected at day −1 failed to respond to SARS-CoV-2-S stimulation. Following immunization, AGMs immunized with CVXGA1 and CVXGA3 generated a cellular response that is SARS-CoV-2 S protein-specific. No SARS-CoV-2-specific cellular response was detected in the BLB control animals (group 6), the cellular responses from animals immunized with CVXGA1 and CVXGA3 increased at each time point post-immunization, Days 7, 14 and 28 (FIGS. 7A-7B). CVXGA3 seems to induce a slightly higher cellular response than CVXGA1.

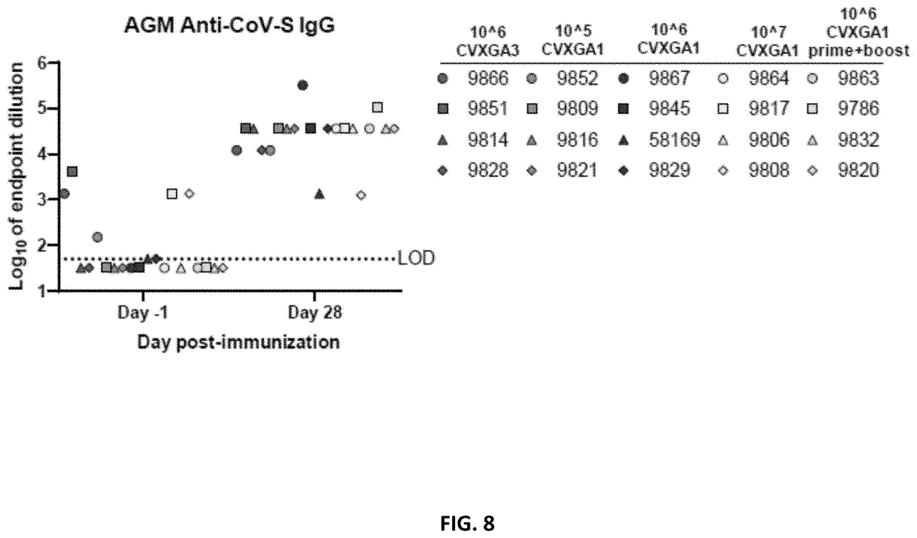

SARS-CoV-2-S IgG antibody response. At −1 and 28 dpi, anti-SARS-CoV-2-S IgG antibodies were quantified. Interestingly, two CVXGA3- and three CVXGA1-immunized AGMs had detectable anti-S antibodies prior to immunization. For all except one CVXGA1-immunized AGM, the anti-S antibody titers increased from −1 to 28 dpi to over 103. For all CVXGA3-immunized AGMs, the anti-S antibody titers increased from −1 to 28 dpi to over 104. One AGM, who had detectable antibodies prior to immunization with CVXGA1, did not have an increase in its anti-S IgG antibody titer (FIG. 8).

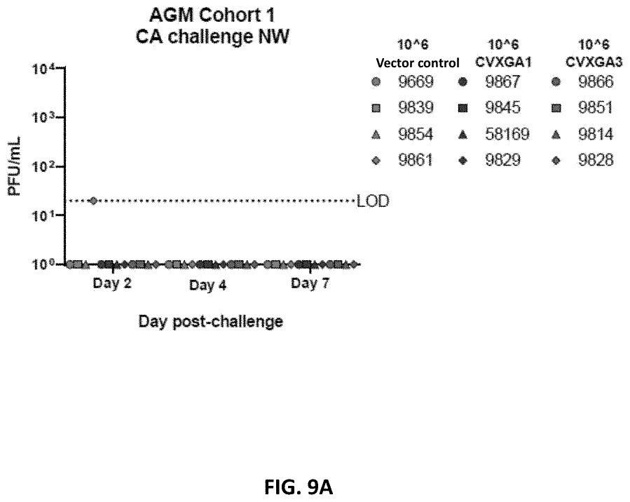

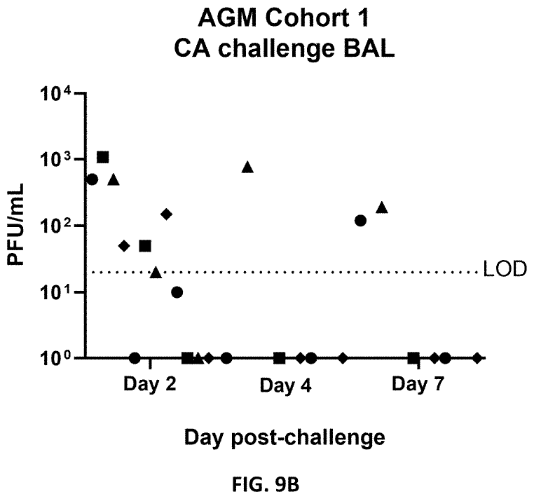

Infectious SARS-CoV-2 challenge virus titer in NW and BAL of vaccinated AGM. At 2, 4, and 7 dpc, NW and BAL were collected and infectious SARS-CoV-2 viral burden was quantified. In the NW, only one AGM, immunized with vector control, had detectable infectious virus (FIG. 9A). However, all four vector control-immunized AGMs had detectable infectious challenge virus in the BAL at 2 dpc with the highest titer being 103 PFU/mL. At 4 and 7 dpc, one of two and two of two vector control-immunized AGMs had detectable virus in their BAL. Three of four CVXGA1-immunized AGMs had detectable infectious virus in their BAL at 2 dpc but not at later time points. In contrast, none of the CVXGA3-immunized AGMs had detectable infectious virus in their BAL at any time point (FIG. 9B).

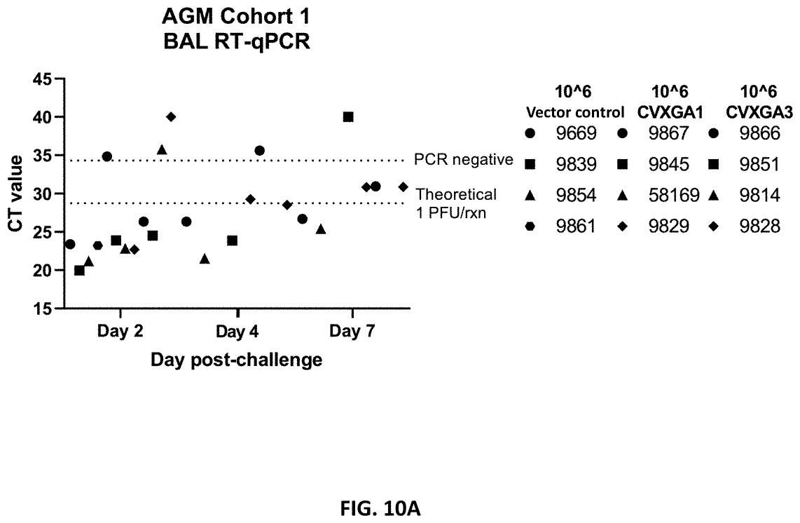

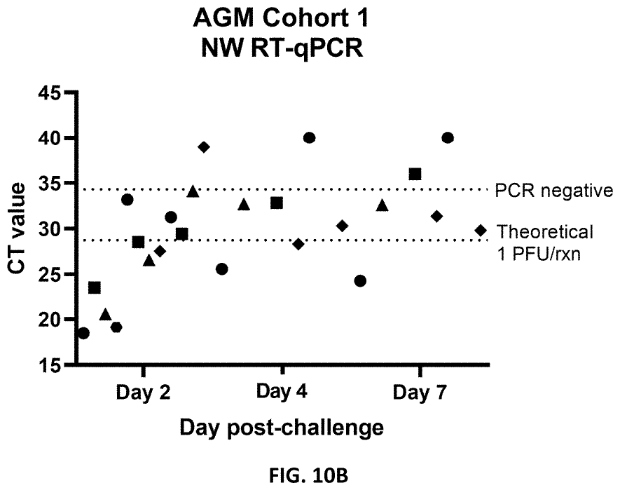

Quantitation of SARS-CoV-2 RNA in the vaccinated AGM NW and BAL following challenge infection. At 2, 4, and 7 dpc, NW and BAL were collected to quantitate SARS-CoV2 challenge virus RNA load by qRT-PCR. Note that a lower cycle threshold (CT) value corresponds to a higher viral titer. AGMs immunized with vector control had CT values corresponding to infectious virus in their BAL at all time points. For CVXGA1-immunized AGMs, three of four at 2 dpc and one of two at 3 dpc had CT values corresponding to infectious virus in their BAL. For CVXGA3, two immunized AGMs had CT values corresponding to infectious virus in their BAL at 2 dpc (FIG. 10A). For the NW, all vector control-immunized AGMs had CT values corresponding to infectious virus at 2 dpc. At 4 and 7 dpc, only half of the vector control-immunized AGMs had CT values corresponding to infectious virus in their NW. Two CVXGA1-immunized AGMs had CT values corresponding to infectious virus at 2 dpc. In contrast, no CVXGA3-immunized AGMs had CT values corresponding to infectious virus in their NW at any time point (FIG. 10B). Compared to the vector control-immunized animals, CVXGA1- and 3-immunized AGMs had greatly reduced challenge virus burden.

Both CVXGA1 and CVXGA3 candidate vaccines induced anti-SARS-CoV-2 S IgG antibodies and SARS-CoV-2 S-specific cellular responses. Furthermore, no infectious SARS-CoV-2 live virus was detected in the NW of CVXGA-immunized AGMs 2, 4, or 7 dpc. For CVXGA3-immunized AGMs, no infectious virus was detected in the BAL. These results were confirmed by the low/no viral RNA burden by qRT-PCR assay, indicating that PIV5 CPI vectored CVXGA candidate SARS-CoV-2 vaccines are effective in providing protection against SARS-CoV-2 alpha variant. CVXGA3 appears to provide a better protection against SARS-CoV-2 alpha variant challenge infection.

### Example 3: Construction and Characterization of PIV5-Vectored SARS-CoV-2 Vaccines

Materials and Methods.

Cells: Vero E6 cells were maintained in Dulbecco's modified Eagle media (DMEM) supplemented with 5% fetal bovine serum (FBS) plus 100 IU/mL penicillin and 100 ug/mL streptomycin (1% P/S; Mediatech Inc, Manassas, Va., USA). Serum-free (SF) Vero cells were maintained in VP-SFM (ThermoFisher Scientific) plus 4 mM GlutaMax (Gibco). Vero-TEMPRSS cells were obtained from Dr. Jeff Hogan, University of Georgia, and maintained in DMEM+10% FBS+1 mg/mL G418. All cells were incubated at 37° C., 5% CO2.

Plasmids and virus rescue: The construction of a plasmid encoding for PIV5 antigenome and generation of recombinant PIV5 has already been described (He, B., et al., Virology, 237(2):249-60 (1997)). To construct plasmids encoding for the antigenome of CVXGA1, CVXGA3, CVXGA5, CVXGA13, and CVXGA14, the Spike (S) genes from SARS-CoV-2 WA1, alpha, gamma, delta, and omicron, respectively, were placed into an additional open reading frame (ORF) between the PIV5 SH and HN genes. The S cytoplasmic tail was replaced by the PIV5 fusion (F) protein cytoplasmic tail. To construct CVXGA2, the SARS-CoV-2 WA1 nucleoprotein (N) gene was placed into an additional ORF between the PIV5 HN and L genes of CVXGA1. Primer sequences are available upon request. To generate recombinant PIV5 viruses CVXGA1, CVXGA2, CVXGA3, CVXGA5, CVXGA13, and CVXGA14, the Neon Transfection System (Invitrogen) was used to electroporate SF Vero cells with 1.9 μg pPIV5-NP, 0.6 μg pPIV5-P, 3.1 μg pPIV5-L, 3.1 μg pT7-polymerase, and 6.3 μg antigenome. Recovered virus was amplified in SF Vero cells, single viral isolates were obtained by limiting dilution or plaque purification, and the viral genomes were verified by RT-PCR and Sanger sequencing.

Virus propagation: The recombinant PIV5 viruses were propagated in SF Vero cells. The cells were infected at a multiplicity of infection (MOI) 0.001 PFU in VP-SFM+4 mM GlutaMax. One hour post-infection, the media was replaced with fresh VP-SFM 4 mM GlutaMax. After 5 to 7 days of incubation at 37° C. with 5% CO2, the media was collected and centrifuged at 1500 rpm for 10 mins to pellet cell debris. The supernatant was mixed with 0.1 volume of 10× sucrose-phosphate-glutamate (SPG) buffer or 10×SPG+10% Arginine, aliquotted, flash-frozen in liquid nitrogen, and stored at −80° C. The PIV5 virus stocks were titrated via immunostain in Vero cells.

The SARS-CoV-2 viruses were propagated in Vero cells with DMEM+1% FBS+1× P/S. WA1 (BEI NR-52281) and alpha variant (BEI NR-54011) were obtained from BEI Resources. The delta variant was provided by Dr. Michael Gale, University of Washington. The omicron variant was provided by Dr. Jeff Hogan, University of Georgia.

Immunofluorescence assay (IFA): To detect PIV5 V/P, SARS-CoV-2 S, and SARS-CoV-2 N, immunofluorescence assays were performed. Vero cells were infected at MOI 0.01 with PIV5, CVXGA1, CVXGA2, CVXGA3, CVXGA5, CVXGA13, or CVXGA14. 3 days post-infection, the cells were fixed with 80% methanol. The cells were incubated with mouse anti-PIV5 V/P, rabbit anti-SARS-CoV-2 S (Sino Biological catalog no. 40150-R007), or SARS-CoV-2 N (ProSci catalog no. 35-579) antibodies at 1:500 in PBS+3% BSA for 1 hr. Next, the cells were washed with PBS and incubated with goat a-mouse Cy3 (KPL) or goat a-rabbit Cy3 (KPL) at 1:500 in PBS+3% BSA for 30 mins. The cells were washed with PBS and imaged with an EVOS M5000 microscope (Thermo Fisher Scientific).

Results

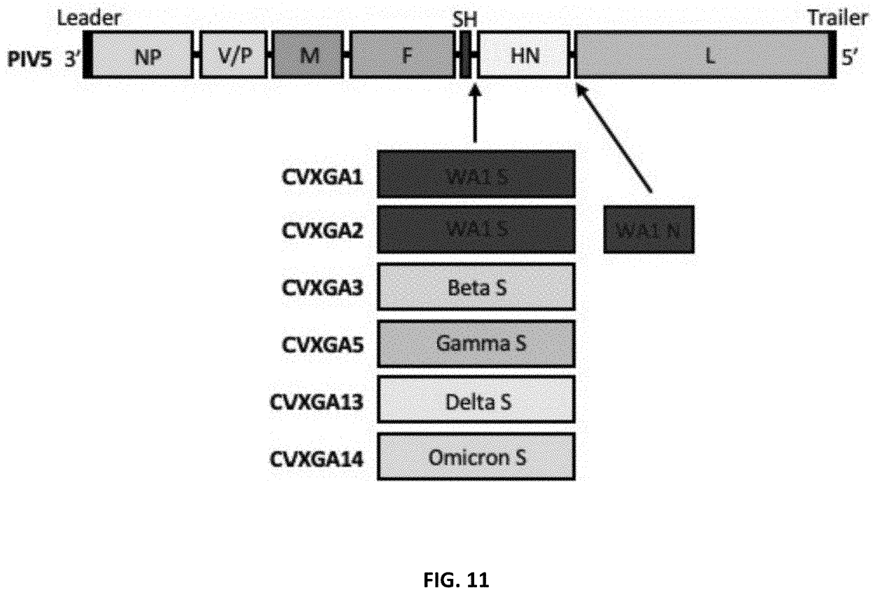

A PIV5-vectored vaccine for SARS-CoV-2 was previously generated by inserting the SARS-CoV-2 WA1 S gene between the PIV5 SH and HN genes (CVXGA1). Additionally, the cytoplasmic tail of the S protein was replaced with the cytoplasmic tail from the PIV5 F protein. A single, intranasal dose of CVXGA1 was shown to protect K18-hACE2 mice from lethal infection with the WA1 strain, the initial circulating strain in the US, and blocks contact transmission in ferrets (An, D., et al., Sci Adv, 7(27) (2021)). To determine whether expressing the N protein of SARS-CoV2 as an additional antigen enhances protection afforded by the S antigen alone, PIV5 expressing both S and N (CVXGA2) was generated. During the study period, SARS-CoV 2 variants of concern (VOC) emerged and some of them became dominant strains circulating in different regions at different times. Thus, PIV5-vectored vaccine candidates expressing S from SARS-CoV-2 VOC were generated in a similar manner as CVXGA1 (FIG. 11): variants alpha (CVXGA3), gamma (CVXGA5), delta (CVXGA13), and omicron (CVXGA14) (collectively called CVXGA vaccines). All variant S genes had the cytoplasmic tail replaced with the PIV5 F cytoplasmic tail.

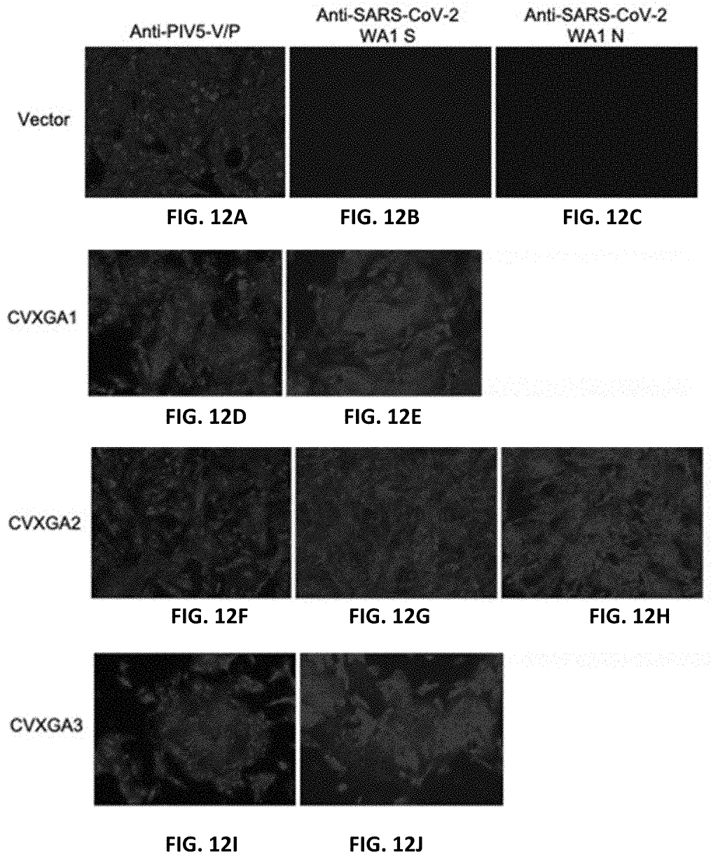

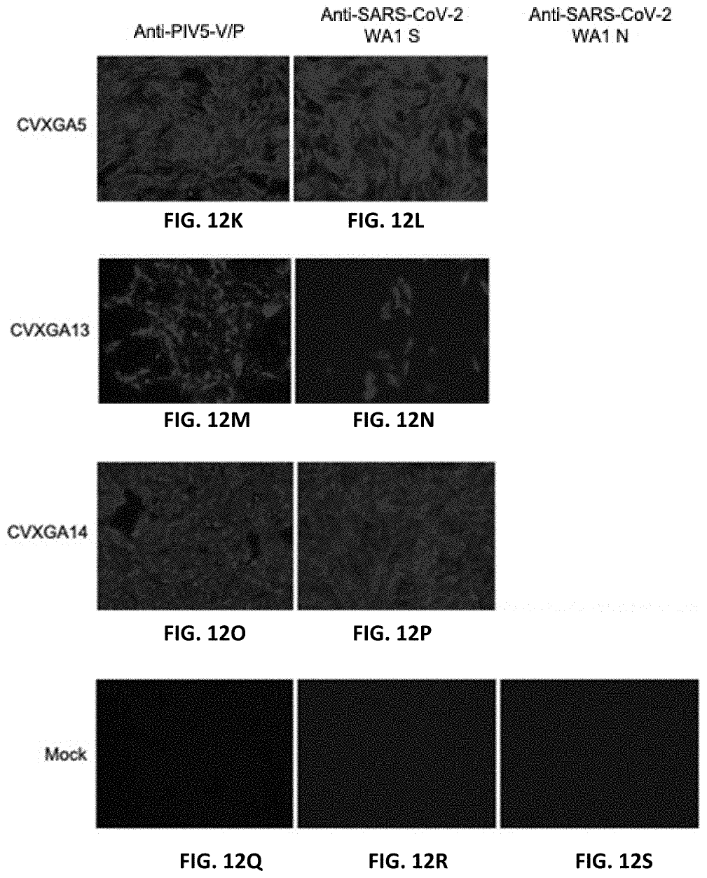

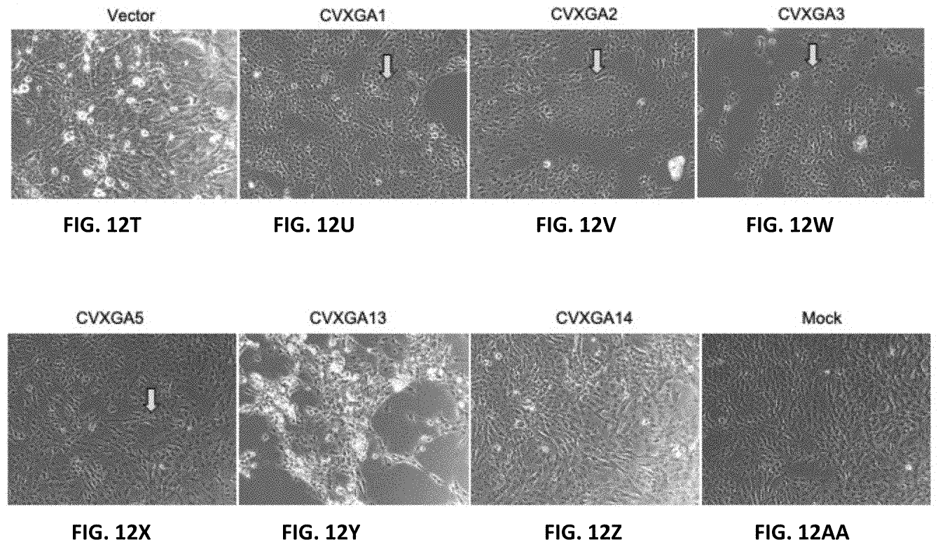

The vaccine viruses were recovered by electroporating the plasmid containing the PIV5 genome with the SARS-CoV-2 antigen along with plasmids encoding for PIV5 NP, P, and L proteins as well as T7 polymerase. The recovered viral genomes were confirmed with RT-PCR and sequencing. To confirm antigen expression, Vero cells were infected at MOI 0.01 with PIV5, CVXGA1, CVXGA2, CVXGA3, CVXGA5, CVXGA13, or CVXGA14 and assayed for immunofluorescence with WA1 S-specific antibody. As expected, S expression was detected in cells infected with CVXGA1, 2, 3, 5, 13 and 14. Additionally, SARS-CoV-2 N expression was only detected in cells infected with CVXGA2 (FIGS. 12A-12S). Compared to PIV5 vector-infected cells, all the PIV5-vectored vaccines had increased syncytia, indicating that SARS-CoV-2-S is functional (FIGS. 12T-12AA).

### Example 4: PIV5-Vectored SARS-CoV-2 Vaccines Induce an Anti-S Humoral Response in Hamsters

Materials and Methods.

Hamsters: Five-to-seven-week-old Golden Syrian hamsters were obtained from Charles River Laboratories. The hamsters were single-housed in biosafety level 2 facilities with ad libitum access to food and water. Pre-challenge procedures were performed at the University of Georgia Biological Sciences Animal Facility. The hamsters were transferred to the University of Georgia Animal Health Research Center for the challenge and post-challenge procedures. The hamsters were anesthetized for immunization, blood collection, and challenge by intraperitoneal injection of 100 μL ketamine/acepromazine cocktail. All experiments were performed in accordance with protocols approved by the Institutional Animal Care and Use Committee at the University of Georgia.

Immunization and challenge of hamsters: The hamsters were anesthetized by intraperitoneal injection of 100 μL ketamine/acepromazine cocktail. To administer intranasal immunizations, anesthetized hamsters were placed on their backs, a pipette was used to dispense 100 μL inoculum onto their noses (50 μL each nostril), and the inoculum was allowed to drain into their respiratory tracts. They were recovered on heating pads.

A Covid-19 mRNA vaccine was obtained from a clinical site after it was thawed and stored at −80° C. 2 μg mRNA vaccine in 50 μL was administered via intramuscular injection.

For study AE19, hamsters (n=8) received a single intranasal immunization of 100 μL of 10⁵ plaque-forming units (PFU) PIV5, CVXGA1, CVXGA2, CVXGA3, or CVXGA5. At 28 dpi, blood was collected from the hamster saphenous vein for serological analysis. At 36 days post immunization (dpi), the hamsters were anesthetized, four hamsters were challenged with 10³ PFU of SARS-CoV-2 Wuhan strain (WA1), and the remaining four hamsters were challenged with 10³ PFU SARS-CoV-2 alpha variant (CA; BEI NR54011). Challenge virus was delivered intranasally with a Teleflex MAD device. Following challenge infection, the hamster weights were monitored for 5 days, the hamster lungs were harvested and homogenized in approximately 1.7 mL sterile PBS, and SARS-CoV-2 viral burden was quantified via plaque assay and real-time quantitative reverse transcription polymerase chain reaction (RT-qPCR).

For study AE23, hamsters received intramuscular immunizations of 100 μL PBS (n=20, group 1) or 2 μg mRNA COVID vaccine (n=20, group 2). At 21 dpi, hamsters that received the mRNA vaccine were boosted with the mRNA vaccine again. At 28 dpi, blood was collected for serological analysis. At 35 dpi following initial immunization, hamsters that received PBS during the first immunization received either 100 μL PBS i.n. (n=5, group 1A), 100 μL 3×10⁵ PFU CVXGA1 (n=5, group 1B), 100 μL 2×10⁵ PFU CVXGA3 (n=5, group 1C), or 100 μL 1.5×10⁵ PFU CVXGA13 (n=5, group 1D). Group 2 hamsters that received two doses of mRNA received 100 μL PBS (n=4, group 2A), 100 μL 3×10⁵ PFU CVXGA1 (n=4, group 2B), 100 μL 2×10⁵ PFU CVXGA3 (n=4, group 2C), 100 μL 1.5×10⁵ PFU CVXGA13 (n=4, group 2D), or a third dose of mRNA (n=4, group 2E). Hamsters were anesthetized for intranasal immunizations but not intramuscular injections. Blood was collected at 54 dpi. At 63 dpi, the hamsters were challenged with 10⁴ PFU SARS-CoV-2 delta variant. Following challenge infection, hamster weights were monitored for 5 days, the hamster lungs were harvested, and SARS-CoV-2 viral burden was quantified via plaque assay and RT-qPCR.

For study AE24, hamsters received 100 μL PBS i.n. (n=5, Group 1), 2 μg mRNA COVID vaccine (n=25, Group 2), or 100 μL 7×10⁴ PFU CVXGA1 (n=10, Groups 3 & 4). At 29 dpi, hamsters that received the mRNA vaccine were boosted with the mRNA vaccine again and group 3 hamsters received another dose of CVXGA1. At 91 dpi following initial immunization, hamsters who received two doses of mRNA received 2 μg mRNA vaccine (n=5, Group 2A), 100 μL 7×10⁴ PFU CVXGA1 (n=5, Group 2B), 100 μL 10³ PFU CVXGA13 (n=5, Group 2C), 100 μL PBS i.n. (n=5, Group 2D), or 100 μL 10⁴ PFU CVXGA14 (n=5, Group 2E). Hamsters were anesthetized for intranasal immunizations but not intramuscular injections. Blood was collected at 36 and 108 dpi. At 116 dpi, the hamsters were challenged with 10⁴ PFU SARS-CoV-2 delta variant. Following challenge infection, hamster weights were monitored for 5 days, the hamster lungs were harvested, and SARS-CoV-2 viral burden was quantified via plaque assay and RT-qPCR.

All animal experiments were performed according to the protocols approved by the Institutional Animal Care and Use Committee at the University of Georgia.

Enzyme-linked immunosorbent assay (ELISA): To quantify the anti-SARS-CoV-2 S and RBD humoral response, hamster serum was analyzed via ELISA. Immulon® 2HB 96-well microtiter plates were coated with 100 μL SARS-CoV-2 S or RBD at 1 μg/mL. For all ELISAs, plates were coated with SARS-CoV-2 S and RBD from the WA1 strain. S and RBD were purified as described previously. (An, D., et al., Sci Adv, 7(27) (2021)). Hamster serum was serial diluted two-fold and incubated on the plates for 2 hrs. Horseradish peroxidase-labelled goat anti-mouse IgG secondary antibody (Southern Biotech, Birmingham, Ala.) was diluted 1:2000 and incubated on the wells for 1 hr. The plates were developed with KPL SureBlue Reserve TMB Microwell Peroxidase Substrate (SeraCare Life Sciences, Inc., Milford, Mass.), and OD450 values were detected with a BioTek Epoch Microplate Spectrophotometer (BioTek, Winooski, Vt.). Antibody titers were calculated as log 10 of the highest serum dilution at which the OD450 was greater than two standard deviations above the mean OD450 of naïve serum.

Results.

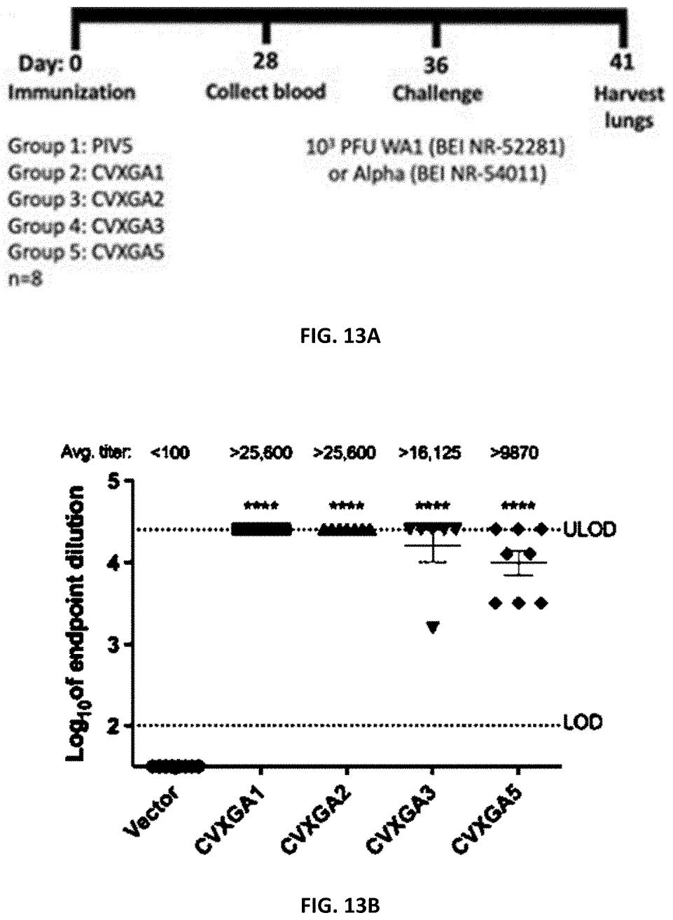

Golden Syrian hamsters are susceptible to SARS-CoV 2 infection. (Chan, J. F., et al., Clin Infect Dis, 71(9):2428-2446 (2020)) To test efficacy of new PIV5-vectored COVID-19 vaccines in hamsters, their ability to induce an immune response in hamsters was examined. Golden Syrian hamsters were immunized with a single, intranasal dose of 10⁵ plaque-forming units (PFU) PIV5 vector, CVXGA1, CVXGA2, CVXGA3, or 5×10² PFU CVXGA5 (FIG. 13A). 28 days post-immunization (dpi), blood was collected from the hamsters and serum anti-SARS-CoV-2-S IgG antibodies were quantified by ELISA. While hamsters immunized with PIV5 vector had no detectable anti-SARS-CoV-2-S antibodies, a single intranasal dose of CVXGA1, CVXGA2, and CVXGA3 induced mean antibody titers of over 10,000, the upper limit of detection for the assay. Even CVXGA5, at a lower immunization dose, was able to induce an anti-S ELISA titer over 9,870 (FIG. 13B).

### Example 5: CVXGA Vaccines Protect Against Homologous and Heterologous Challenges

Materials and methods.

Neutralization assays: To quantify the SARS-CoV-2-neutralizing antibodies generated by the hamsters, microneutralization assays were performed. Hamster serum was heat-inactivated at 56° C. for 45 mins and serially diluted two-fold. The serum was mixed 1:1 with 6×103 focus-forming units (FFU)/mL SARS-CoV-2 WA1, delta, or omicron. The serum/virus mixture was incubated at 37° C. for 1 hr before being incubated on 96-wells of Vero cells for WA1 or Vero TEMPRSS2 cells for delta and omicron. One hour post-infection, a methylcellulose overlay (Dulbecco's modified Eagle medium (DMEM)+5% fetal bovine serum (FBS)+1% penicillin/streptomycin+1% methylcellulose) was added on top of the serum/virus mixture. The plates were incubated at 37° C., 5% CO2 for 24 hrs. The methylcellulose overlay was removed, the wells were washed with PBS, and the cells were fixed with 60% methanol/40% acetone. The number of infected cells were quantified via immunostain. Neutralization titers were calculated as log 10 of the highest serum dilution at which the virus infectivity was reduced by at least 50%.

Plaque assay for infectious virus: Following euthanasia, whole hamster lungs were removed, added to approximately 1.7 mL sterile PBS, and homogenized. To quantify infectious SARS-CoV-2, plaque assays were performed with lungs from AE19 and AE23, and FFU assays were performed with lungs from AE24.

For the plaque assays, lung homogenates were serially diluted in Dulbecco's modified Eagle medium (DMEM)+2% fetal bovine serum (FBS)+1% antibiotic/antimycotic and added to 12-well plates of Vero E6 cells for SARS-CoV-2 WA1 and alpha variant or Vero TEMPRSS2 cells for delta and omicron variants. At 1 hour post-infection, the inoculum was removed, and a methylcellulose overlay (500 mL Opti-MEM+0.8% methylcellulose+2% FBS+1% antibiotic/antimycotic) was added to the wells. The plates were incubated for 3 days, the overlay was removed, and the cells were fixed with 60% methanol/40% acetone. The cells were stained with crystal violet, the number of plaques were counted, and viral titers are expressed as PFU/mL of lung homogenate.

qPCR: SARS-CoV-2 viral RNA levels were quantified by RT-qPCR. SARS-CoV-2 virus was inactivated by mixing 100 μL lung homogenate with 900 μL TRIzol (Invitrogen). Using a QIAgen RNA extraction kit, RNA was extracted from 140 μL homogenate/TRIzol and eluted in 15 μL of elution buffer, of which 5 μL was used in the qRT-PCR reaction. qRT-PCR was performed according to the protocol described in the “CDC 2019-Novel Coronavirus (2019-nCoV) Real-Time RT-PCR Diagnostic Panel . . . Instructions for Use” (page 26) with Applied Biosystems TaqPath One Step RT qPCR Master Mix and SARS-CoV-2 Research Use Only qPCR Primer and Probe Kit primer/probe mix N1. SARS-CoV-2 viral RNA was extracted from SARS-CoV-2 WA1, alpha variant, and delta variant viruses of known titers. RNA was extracted from 140 μL virus and eluted in 15 μL of elution buffer. The viral RNA was serially diluted 10-fold and 5 μL from each dilution was used in the RT-qPCR assay. To generate a standard curve, the viral titer was plotted on the x-axis and the CT value was plotted on the y-axis. The standard curve was used to calculate the CT value that corresponds to 1 PFU/rxn. The standard curve was also used to calculate PFU/rxn from the CT values of homogenized lung RNA samples. The CT value of RNA extracted from sterile elution buffer was designated the PCR negative cutoff

Results.

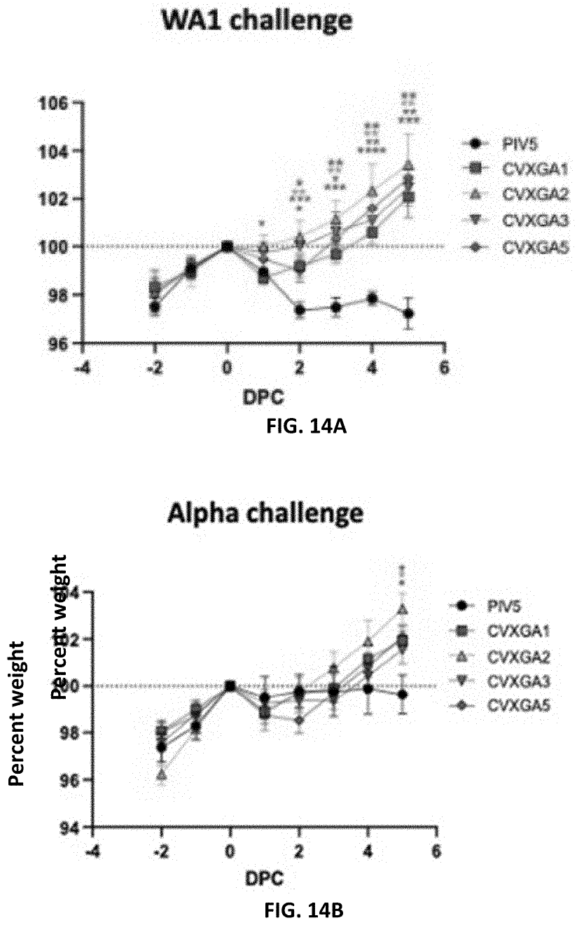

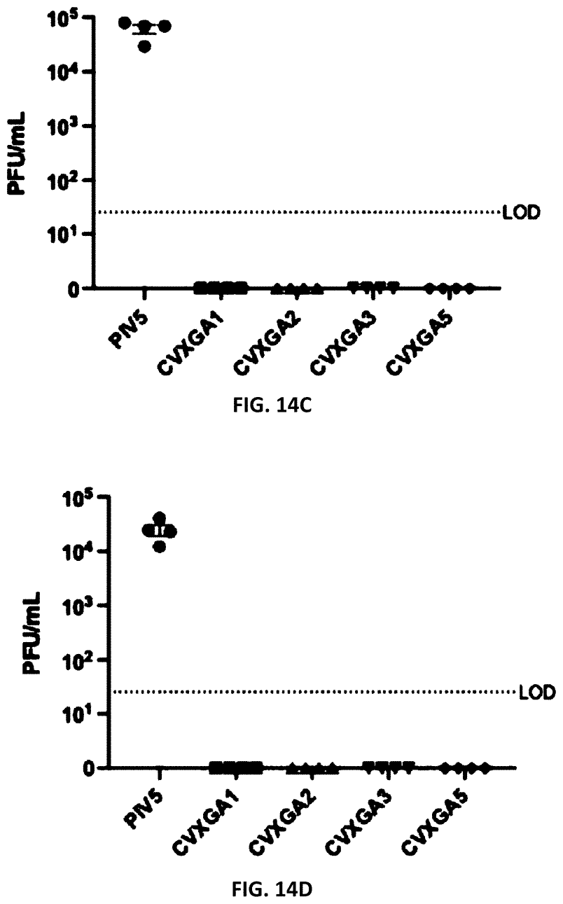

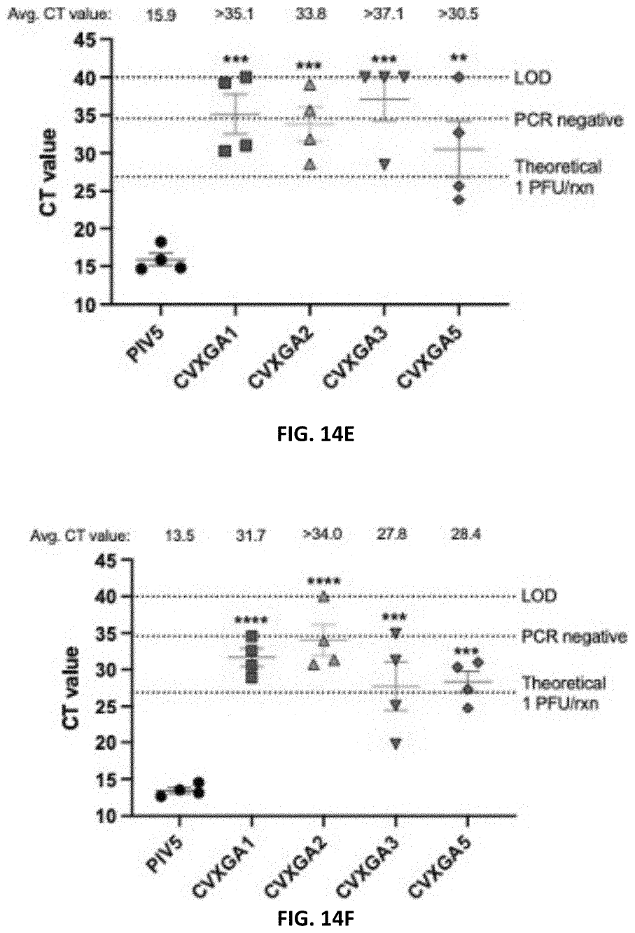

To assess the efficacy of PIV5-vectored SARS-CoV-2 vaccines against homologous and heterologous challenges, CVXGA-immunized hamsters were challenged with either 103 PFU SARS-CoV-2 WA1 (USA-WA01/2020) or alpha variant (CA; BEI NR54011) at 36 dpi (FIG. 13A). The hamster weights were monitored daily for 5 days post-challenge (dpc). Following challenge with WA1, hamsters immunized with PIV5 vector lost weight and did not recover before the study was terminated. In contrast, hamsters immunized with CVXGA1, CVXGA2, CVXGA3, or CVXGA5 lost weight at day 1 post-challenge but returned to pre-challenge weights by 3 or 4 dpc (FIG. 14A). While challenge with alpha variant induced less severe weight loss in PIV5 vector-immunized hamsters, hamsters immunized with CVXGA1, CVXGA2, CVXGA3, or CVXGA5 had significantly higher body weights compared to PIV5 vector-immunized hamsters at 5 dpc. In both virus challenge groups, hamsters immunized with CVXGA2 had the highest mean body weight gains following challenge (FIG. 14B), suggesting that N as an additional antigen provided protection.

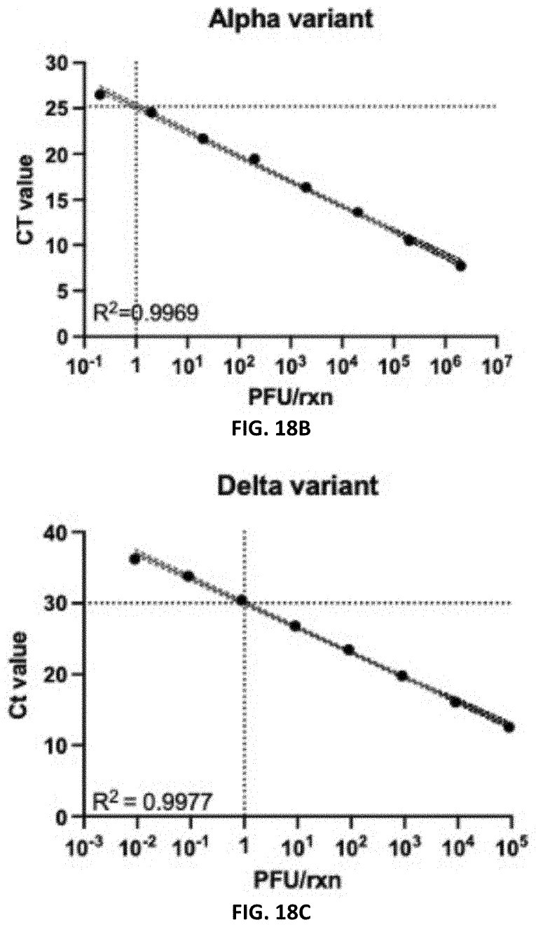

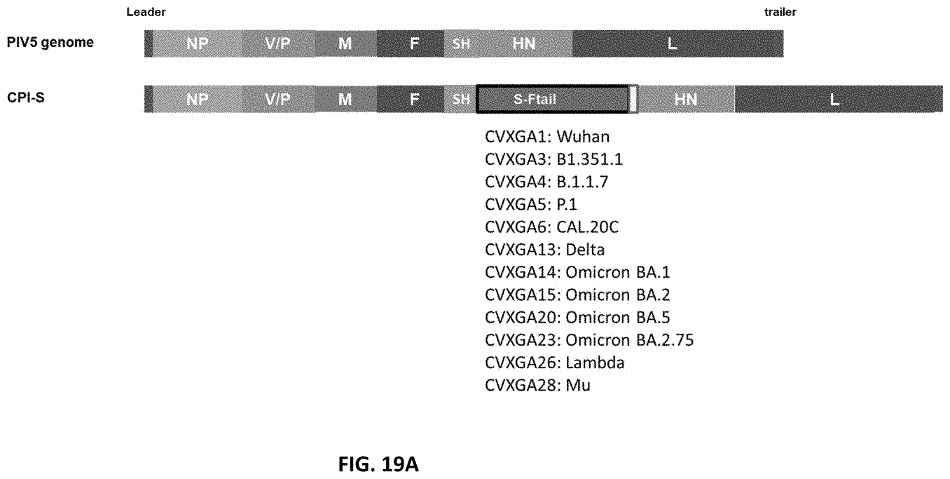

To quantify viral burden in the lungs, hamster lungs were harvested and homogenized 5 dpc. Infectious virus was quantified by plaque assay. Hamsters immunized with PIV5 vector had infectious virus of over 4 log 10 PFU/mL lung homogenate following challenge with WA1 or alpha variant, while no infectious WA1 or alpha variant was detected in hamsters immunized with CVXGA1, CVXGA2, CVXGA3, or CVXGA5 (FIGS. 14C-14D; limit of detection, LOD, 25 PFU/mL). Viral RNA in the lung, were detected with RT-qPCR. Hamsters immunized with PIV5 vector and challenged with WA1 and alpha variant had mean cycle threshold (CT) values of 15.9 and 13.5, respectively (FIGS. 14E-14F). Following challenge with WA1 or alpha variant, hamsters that received a single intranasal dose of CVXGA1 or CVXGA2 had CT values indicative of less than 1 PFU per reaction (PFU/rxn). Interestingly, CVXGA3-immunized hamsters had CT values indicative of less than 1 PFU/rxn following challenge with WA1 but two hamsters had CT values equating to 1 and 96 PFU/rxn following challenge with alpha variant (FIGS. 14E-14F, FIGS. 18A-18C). A single dose of CVXGA2 performed the best against heterologous challenge with alpha variant (FIG. 14F), again, suggesting that SARS-CoV-2 N might be protective.

### Example 6: CVXGA Vaccines Protect Against Delta Challenge

Results.

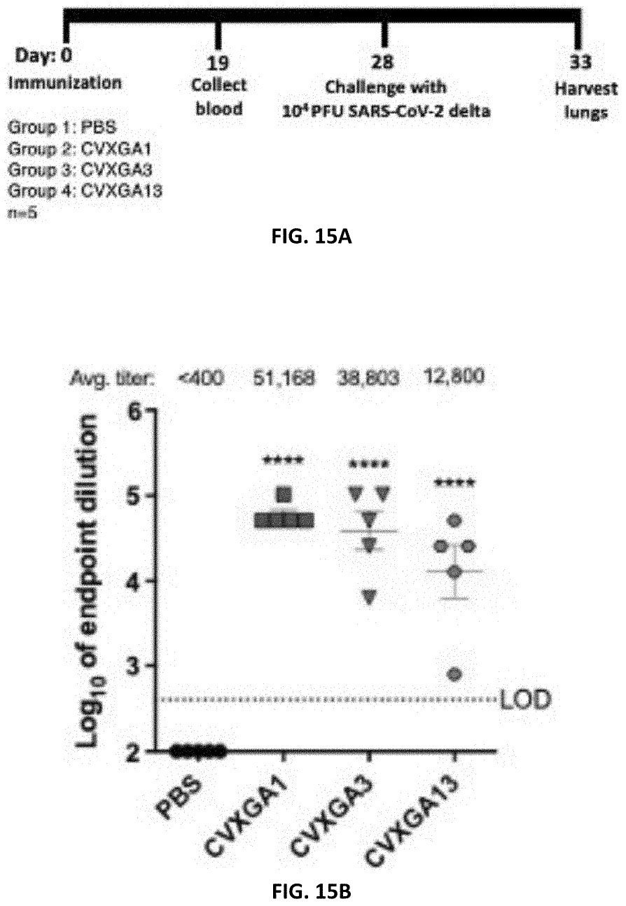

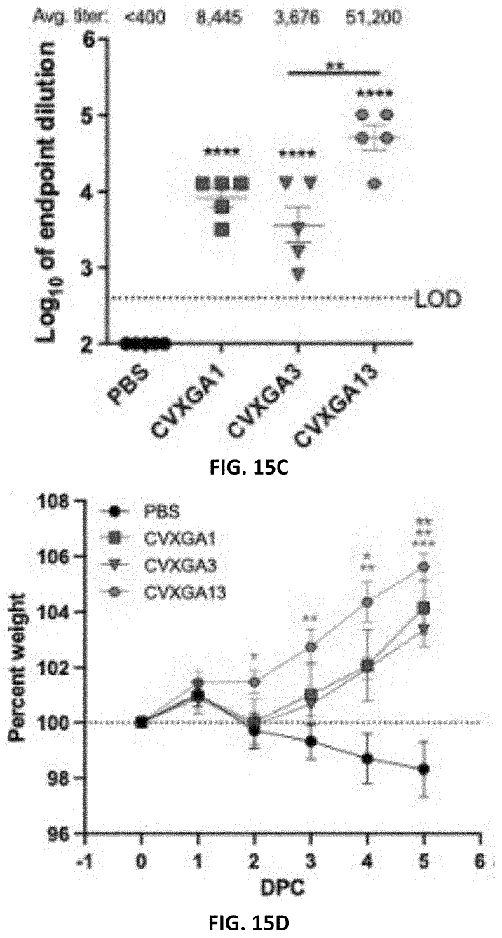

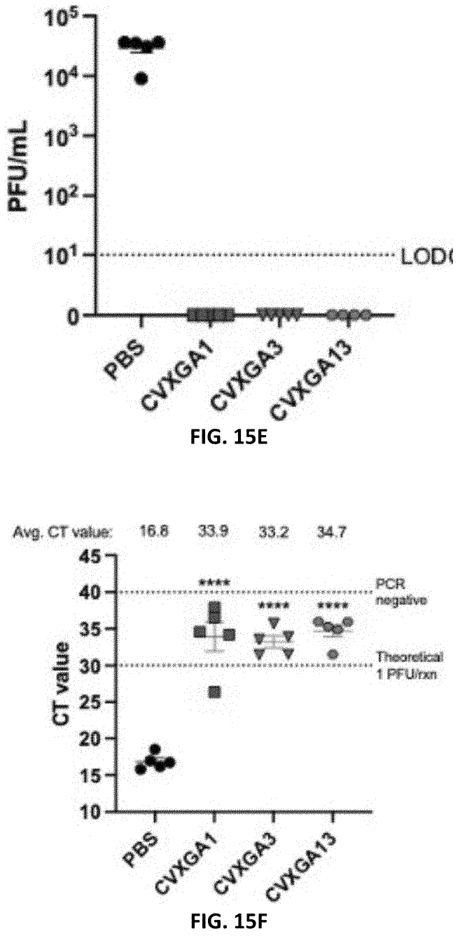

As of May 2022, delta variant is one of two VOCs circulating in the United States. Therefore, we assessed the efficacy of our primary vaccine candidate, CVXGA1, against heterologous challenge with delta variant and test a vaccine expressing S from delta variant against homologous challenge. Hamsters received a single, intranasal dose of PBS or 105 PFU CVXGA1, CVXGA3, or CVXGA13. 19 dpi, blood was collected and serum anti-SARS-CoV-2 WA1 S and -RBD IgG antibodies were quantified via ELISA. Immunization with CVXGA1, CVXGA3, and CVXGA13 elicited anti-S antibodies with mean titers of over 10,000 (FIG. 15B). Interestingly, hamsters immunized with CVXGA13 had the highest quantity of anti-WA1 RBD antibodies with a mean titer of 51,200 (FIG. 14C).

At 28 dpi, the hamsters were intranasally challenged with 10⁴ PFU SARS-CoV-2 delta variant and their weights were monitored for 5 days. Beginning at 2 dpc, hamsters immunized with PBS experienced weight loss that steadily declined until the study was terminated. In contrast, hamsters immunized with CVXGA1, CVXGA3, or CVXGA13 experienced weight gain after 2 days post-challenge. While not statistically significant, hamsters immunized with CVXGA13 had greater weight gain than hamsters immunized with CVXGA1 or CVXGA3, indicating that PIV5 expressing S from SARS-CoV-2 delta variant protected hamsters best from weight loss following homologous challenge with delta variant (FIG. 15D). To further assess the vaccine efficacy, lungs were harvested at 5 dpc and subjected to plaque assay and RT-qPCR. While hamsters immunized with CVXGA1, CVXGA3, or CVXGA13 did not have detectable infectious challenge virus in their lung homogenates, hamsters immunized with PBS had a mean titer of 3×104 PFU/mL lung homogenate (FIG. 15E). SARS-CoV-2 delta RNA was detected in lung homogenates via RT-qPCR. Four of five hamsters immunized with CVXGA1 and all hamsters immunized with CVXGA3 or CVXGA13 had CT values indicative of no infectious virus. One CVXGA1-immunized hamster had a CT value of 26.39 (FIG. 15F). The weight loss and lung viral burden data from this study indicate that single, intranasal doses of CVXGA1 and CVXGA3 protect hamsters from heterologous challenge and CVXGA13 protects hamsters from homologous challenge with delta variant.

### Example 7: CVXGA Vaccines Boost the Antibody Response Induced by a mRNA COVID-19 Vaccine

Results.

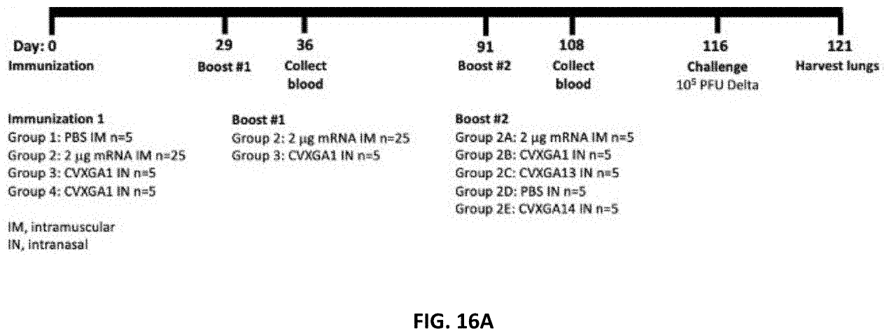

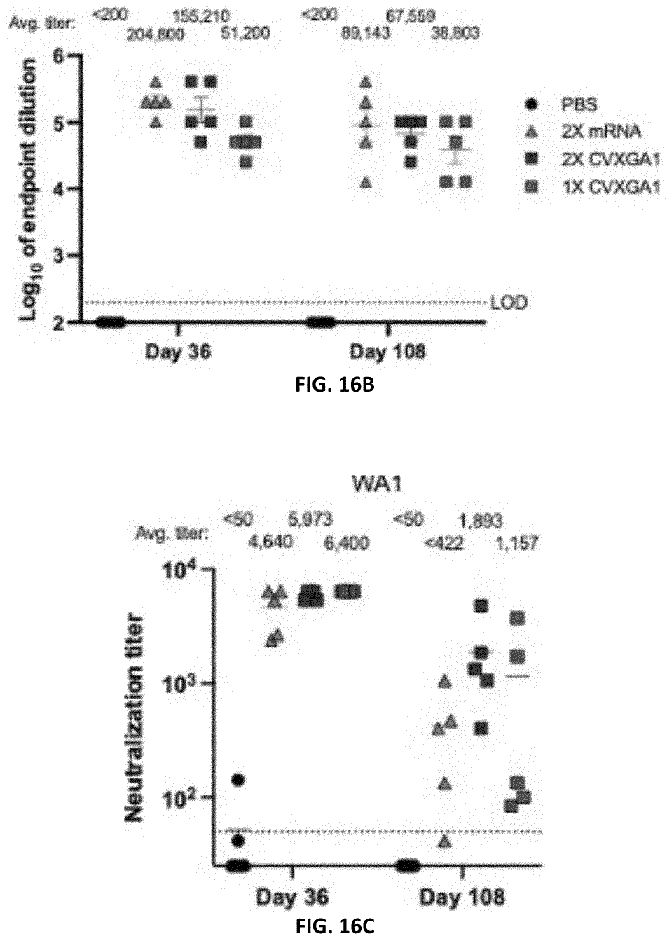

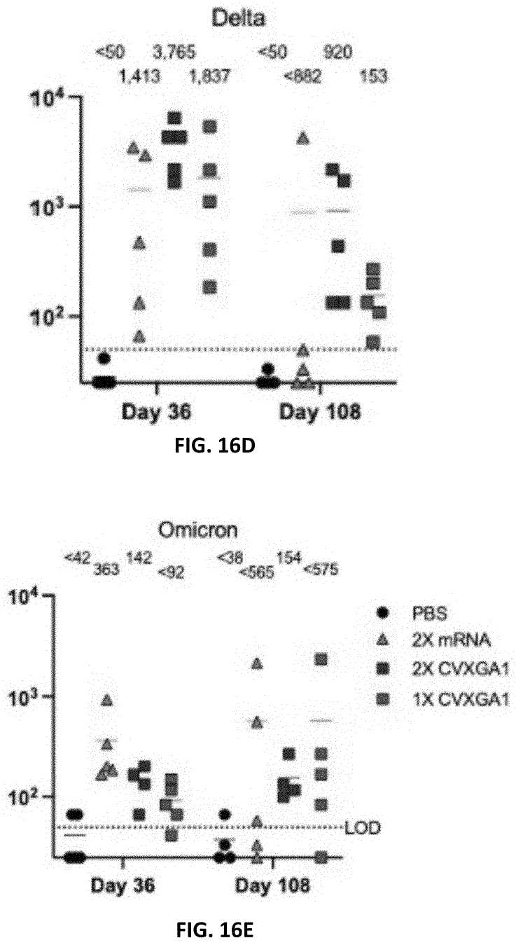

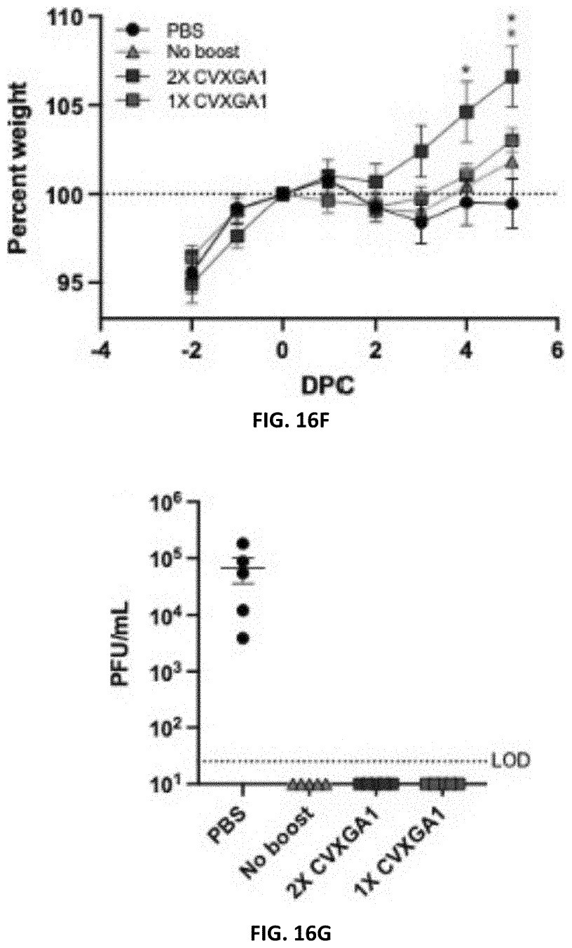

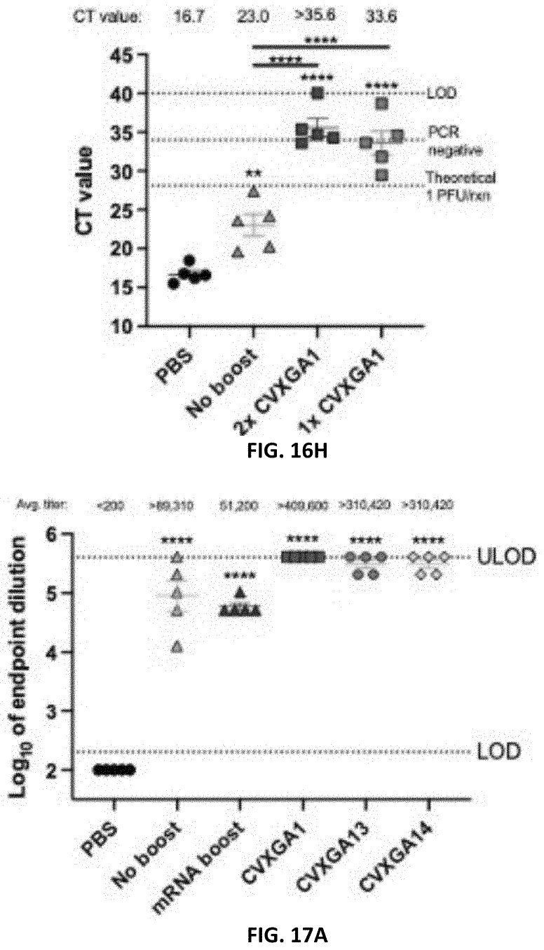

As of May, 2022, 67 percent of individuals in the United States are fully vaccinated against COVID-19. However, data suggests that vaccine-induced immunity wanes over time and is less effective against SARS-CoV-2 variants (Planas, D., et al., Nature, 596(7871): 276 (2021); Zhang, L., et al., Emerg Microbes Infect, 11(1): 1 (2022); Feikin, D. R., et al., Lancet, 399(10328): 924 (2022)). To assess the efficacy of CVXGA1, we compared one (1×CVXGA1) and two (2×CVXGA1) intranasal doses of CVXGA1 to two intramuscular doses of a mRNA COVID-19 vaccine (2× mRNA) (FIG. 16A). Blood was collected 7 days following the second immunization and anti-SARS-CoV-2-S IgG antibodies were quantified via ELISA. Hamsters who received 2× mRNA had highest anti-S ELISA titer, 2×CVXGA1-immunized hamsters had higher anti-S titers than 1×CVXGA1-immunized hamsters, whose mean anti-WA1 S antibody titer was 51,200 (FIG. 16B). To assess the neutralizing ability of the antibodies, the hamster serum was used in microneutralization assays with SARS-CoV-2 WA1, delta variant, and omicron variant. While 2× mRNA generated a high level of anti-WA1 neutralizing antibody (4,640 at 7 days post-second dose), both 2× and 1×CVXGA1 generated comparable levels of anti-WA1 neutralization antibody even though they had lower levels of anti-S ELISA titers (FIGS. 16B-16E). Consistent with reduced cross-reactivity of mRNA-generated antibodies with delta and omicron VOC, delta- and omicron-neutralizing levels were lower than anti-WA1 in hamsters immunized with 2× mRNA at 1,413 and 363 respectively at 7 days post-second dose. As expected, 1× and 2×CVXGA1 generated anti-delta neutralization levels that were moderately lower than anti-WA1; however, 1× and 2×CVXGA1-generated anti-omicron neutralization levels were significantly lower (FIGS. 16C-16E).

To compare longevity of antibody responses in the hamsters, 79 days after boost (day 108 after initial immunization) sera were collected. Anti-S ELISA titers dropped over this time period by 56.5, 56.5, and 24.3 percent for 2× mRNA, 2×CVXGA1, and 1×CVXGA1, respectively (FIG. 16B). Reduction of neutralizing antibody titers in 2× mRNA-immunized hamsters was substantial with 20, 60, and 40 percent of hamsters having no detectable WA1-, delta-, and omicron-neutralizing antibodies 79 post-boost. In contrast, serum from all hamsters immunized with 2×CVXGA1 maintained levels of neutralizing antibodies against WA1, delta variant, and omicron variant (FIGS. 16C-16E).

87 days after the second immunization, the hamsters were challenged with delta variant and the hamster weights were monitored for five days. Compared to hamsters who were immunized with PBS, hamsters who received two doses of CVXGA1 had significant weight gain following challenge. In contrast, hamsters who received two doses of mRNA vaccine did not experience weight gain that was statistically significant (FIG. 16F). To quantify the viral burden in the hamster lungs post-challenge, lungs were collected and homogenized at 5 dpc. While PBS-immunized hamsters had mean lung titers of 5.2×103 FFU/mL lung homogenate, none of the vaccinated hamsters had detectable infectious virus in their lung homogenate (FIG. 16G). However, RT-qPCR revealed that all hamsters immunized with two doses of mRNA vaccine had SARS-CoV-2 vRNA levels indicative of infectious virus with the mean CT value equating to 38 PFU per RT-qPCR reaction (FIG. 16H).

### Example 8: Boosting with CVXGA1 Improves Protection of Hamsters Immunized with mRNA COVID-19 Vaccine

Results.

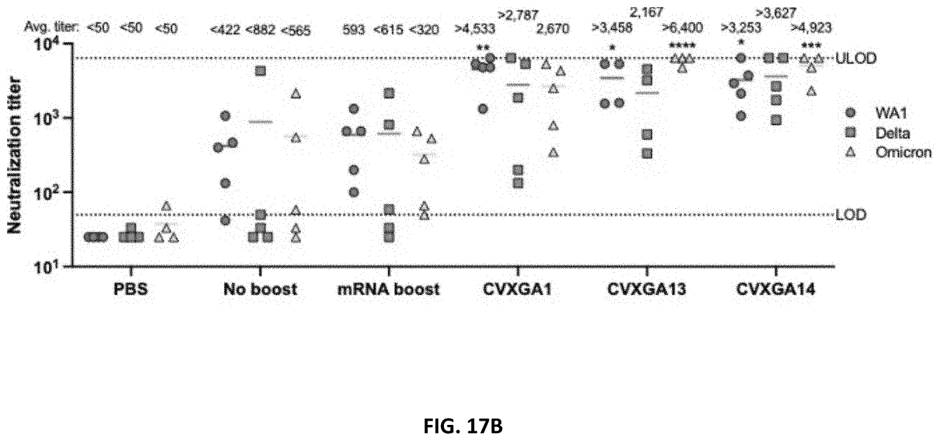

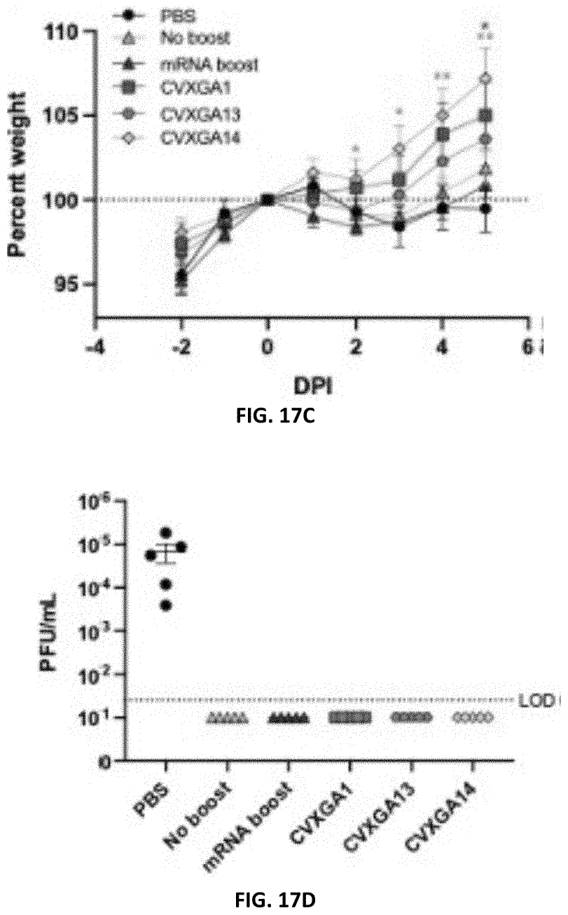

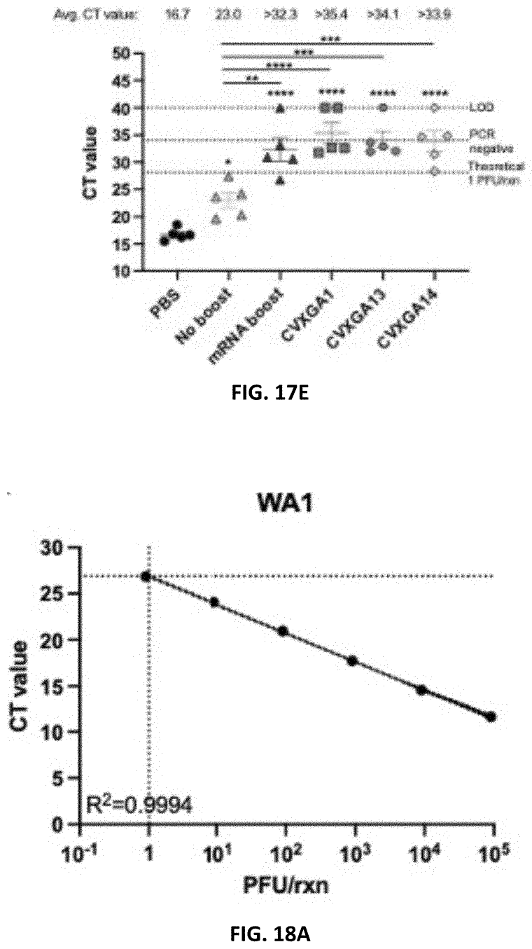

Due to large populations having already immunized with COVID-19 vaccines, we investigated the use of CVXGA vaccines as a boost (FIG. 16A). We used 2× mRNA immunization as a starting point for comparison. Hamsters were first immunized with two doses of mRNA COVID-19 vaccine. 62 days after second dose of mRNA vaccination, hamsters were boosted with mRNA (total 3×mRNA vaccine doses), CVXGA1, CVXGA3, CVXGA14, or PBS. Seventeen days after the third immunization, blood was collected from the hamsters and anti-WA1 S IgG antibodies were quantified via ELISA. Hamsters who received intranasal boosts of CVXGA1 (group 2B), CVXGA13 (group 2C), or CVXGA14 (group 2E) had anti-S IgG titers over 5 log 10, while hamsters who received a mRNA vaccine boost (group 2A) or no boost (group 2D) had lower anti-S IgG titers (FIG. 17A). In neutralization assays with WA1, delta variant, and omicron variant, there was negligible difference in average neutralization titer between sera from hamsters who received a mRNA boost to hamsters who received no boost. However, boosting with mRNA increased detectable levels of neutralizing anti-WA1, delta and omicron from 4, 2 and 3 of 5 animals to 5, 3 and 5 of 5 respectively, indicating that a mRNA boost modestly increased neutralizing antibody responses in the animals (FIG. 17B). In contrast, significant levels of neutralization antibody against WA1, delta variant, and omicron variant were observed in hamsters boosted with CVXGA1, CVXGA13, and CVXGA14 (FIG. 17B).

Twenty-five days following the boost, the hamsters were challenged with delta variant. Hamsters who received intranasal boosts of CVXGA14 or CVXGA1 had the best weight gain compared to hamsters received PBS, no boost, or an mRNA boost. Interestingly, hamsters who received CVXGA14 experienced higher weight gain than hamsters who received CVXGA13 (homologous delta S antigen) (FIG. 17C). Lungs were harvested at five days post-challenge and infectious virus was quantified. None of the boosted hamsters had detectable infectious virus in their lung homogenate (FIG. 17D; LOD, 25 PFU/mL). However, high levels of viral RNA were detected in animals with only two doses of mRNA. CVXGA1-boosted animals had the least amount of viral RNA and mRNA-boosted animals had the most amount of viral RNA among all boosted groups (FIG. 17E).

### Example 9: SARS-COV-2 Vaccine Candidates

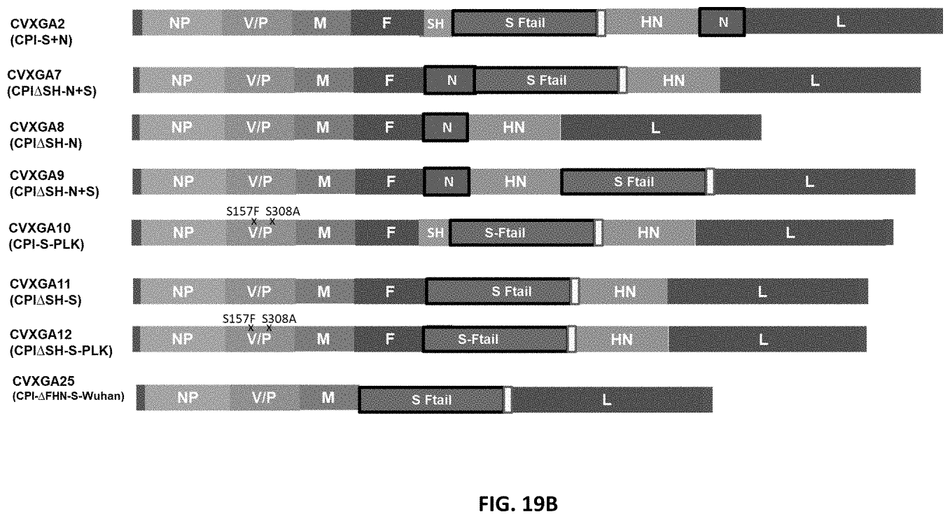

A number of novel SARS-COV-2 vaccine candidates were rescued as described below and summarized in FIGS. 19A-19B and Table 4.

TABLE 4
SARS-CoV-2 vaccine candidate list

| Plasmid | | PIV5 | virus | SARS-CoV-2 strain, inserted gene | Virus |
|---|---|---|---|---|---|
| name | vector | backbone | name | and location, backbone modifications | obtained |
| pCVL6 | pUC | CPI | CVXGA1 | S from Wuhan strain inserted between SH and HN (made | Ph1 |
| | | | | in UGA lab) | |
| pCVL34 | pKBS3 | CPI | CVXGA2 | S and N from Wuhan strain, S is between SH and HN, N | √ |
| | | | | is between HN and L | |
| pCVL44 | pUC | CPI | CVXGA3 | S from Beta variant inserted between SH and HN | √ |
| pCVL48 | pUC | CPI | CVXGA4 | S from Alpha variant inserted between SH and HN | √ |
| pCVL49 | pUC | CPI | CVXGA5 | S from Gamma variant inserted between SH and HN | √ |
| pCVL50 | pUC | CPI | CVXGA6 | S from Epsilon variant inserted between SH and HN | √ |
| pCVL52 | pUC | CPI | CVXGA7 | N from Wuhan strain, S from Beta variant, N and S are | Plasmid |
| | | | | between F and HN, backbone is CPI lacking SH. | construction |
| pCVL53 | pUC | CPI | CVXGA8 | N from Wuhan strain inserted between F and HN, | √ |
| | | | | backbone is CPI lacking SH. | |
| pCVL54 | pUC | CPI | CVXGA9 | N from Wuhan strain inserted between F and HN, S from | √ |
| | | | | Beta variant inserted between HN and L, backbone is CPI | |
| | | | | lacking SH. | |
| pCVL55 | pUC | CPI | CVXGA10 | S from Beta variant inserted between SH and HN, | √ |
| | | | | backbone is CPI containing S157F and S308A in V/P | |
| | | | | gene. | |
| pCVL58 | pUC | CPI | CVXGA11 | S from Beta variant inserted between F and HN, backbone | Rescue |
| | | | | is CPI lacking SH. | |
| pCVL59 | pUC | CPI | CVXGA12 | S from Beta variant inserted between F and HN, backbone | Rescue |
| | | | | is CPI lacking SH and containing S157F and S308A in | |
| | | | | V/P gene. | |
| pCVL60 | pUC | CPI | CVXGA13 | S from Delta variant inserted between SH and HN | √ |
| pCVL64 | pUC | CPI | CVXGA26 | S from Lambda variant inserted between SH and HN | Rescue |
| pCVL65 | pUC | CPI | CVXGA27 | N from Wuhan strain inserted between F and HN, S from | Plasmid |
| | | | | Delta variant inserted between HN and L, backbone is | construction |
| | | | | CPI lacking SH. | |
| pCVL67 | pUC | CPI | CVXGA28 | S from Mu variant inserted between SH and HN | Plasmid |
| | | | | | construction |
| pCVL80 | pUC | CPI | CVXGA14 | S from Omicron BA1 strain inserted between SH and HN | √ |
| | | | | in CPI backbone | |
| pCVL86 | pUC | CPI | CVXGA15 | S from Omicron BA2 strain inserted between SH and HN | rescue |
| | | | | in CPI backbone | |
| pCVL108 | pUC | CPI | CVXGA20 | S from Omicron BA.5 variant inserted between SH and | √ |
| | | | | HN in CPI backbone | |
| pCVL111 | pUC | CPI | CVXGA23 | S from Omicron BA.2.75 variant inserted between SH | Plasmid |
| | | | | and HN in CPI backbone | construction |
| pCVL113 | pUC | CPI | CVXGA25 | PIV5 F and HN genes are deleted. S from Wuhan strain | Plasmid |
| | | | | inserted between M and L in CPI backbone. | construction |

SARAS-COV-2 variants representing variants of concern or variants of interest were produced by inserting the Spike (S) protein gene from different variants into PIV5 canine parainfluenza virus (CPI) vector: CVXGA1, 3, 4, 5, 6, 13, 14, 15, 20, 23, 26, and 28.

To improve vaccine efficacy, the N protein gene of SARS-CoV-2 was inserted between the HN and L gene junction in addition to the S inserted at the SH and HN junction, the resulting virus is named as CVXGA2. The expression of both the S and N proteins of SARS-CoV-2 is expected to offer additional protection by eliciting both antibody and cellular immune responses offered by the N protein. However, CVXGA2's titer is low, further improvement is therefore explored in this disclosure.

The mutations in the V/P gene, S157F and 5308A, have been shown previously to increase viral polymerase activities and improve viral titer or yield (Timani K A, et al., J Virol., 82(18):9123(2008); Sun D, et al., PLoS Pathog., 5(7):e1000525 (2009)). We therefore introduced S157F and 5308A into the CPI V/P gene to produce CVXGA10 (CPI-S-PLK) and CVXGA12 (CPIΔSH-S-PLK) viruses.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

We claim:

1. A viral expression vector comprising a parainfluenza virus 5 (PIV5) genome having a heterologous nucleic acid sequence with at least 98% sequence identity to SEQ ID NOs: 61, 62, 63, 64, 65, 66, 67, 68, 69, 70, 71, 72, 73, 74, 75, 76, 77, 78, 79, or 80, wherein the viral expression vector expresses a heterologous polypeptide comprising a coronavirus spike (S) and/or nucleocapsid (N) proteins.

2. The viral expression vector of claim 1, wherein the coronavirus S protein is a coronavirus S protein of the severe acute respiratory syndrome coronavirus 2 (SARS-CoV-2), a variant of interest or a variant of concern of SARS-CoV-2 and the coronavirus N protein is the coronavirus N protein of SARS-CoV-2, a variant of interest or a variant of concern of SARS-CoV-2.

3. The viral expression vector of claim 1, wherein the coronavirus S protein is the coronavirus S protein of a SARS-CoV-2 beta variant, a SARS-CoV-2 gamma variant, a SARS-CoV-2 delta variant, or a SARS-CoV-2 omicron variant and the coronavirus N protein is the coronavirus N protein of a SARS-CoV-2 beta variant, a SARS-CoV-2 gamma variant, a SARS-CoV-2 delta variant, or a SARS-CoV-2 omicron variant.

4. The viral expression vector of claim 1, wherein the coronavirus S protein comprises the coronavirus S protein of SARS-CoV-2 and wherein the cytoplasmic tail of the coronavirus S protein has been replaced with the cytoplasmic tail of the fusion (F) protein of PIV5.

5. The viral expression vector of claim 1, wherein the coronavirus S protein of a variant of SARS-CoV-2 has been inserted between the PIV5 small hydrophobic (SH) and hemagglutinin (HN) genes and the coronavirus N protein of a variant of SARS-CoV-2 has been inserted between the PIV5 HN and polymerase (L) genes.

6. The viral expression vector of claim 1, wherein the coronavirus S protein of a variant of SARS-CoV-2 has been inserted between the PIV5 HN and polymerase (L) genes.

7. The viral expression vector of claim 1, wherein the coronavirus S protein is a S protein from a variant of interest or a variant of concern of SARS-CoV-2 and the coronavirus N protein is a N protein from different variant of interest or a variant of concern of SARS-CoV-2.

8. The viral expression vector of claim 1, wherein the PIV5 small hydrophobic (SH) gene has been replaced with the coronavirus N protein of SARS-CoV-2.

9. The viral expression vector of claim 1, wherein the PIV5 genome further comprises one or more mutations comprising a mutation of the V/P gene, a mutation of the shared N-terminus of the V and P proteins, a mutation of residues 26, 32, 33, 50, 102, and/or 157 of the shared N-terminus of the V and P proteins, a mutation lacking the C-terminus of the V protein, a mutation lacking the small hydrophobic (SH) protein, a mutation of the fusion (F) protein, a mutation of the phosphoprotein (P), a mutation of the large RNA polymerase (L) protein, a mutation incorporating residues from canine parainfluenza virus, a mutation inducing apoptosis, or a combination thereof.

10. The viral expression vector of claim 9, wherein the one or more mutations comprise PIV5VΔC, PIV5ΔSH, PIV5-P-S308G, or a combination thereof.

11. The viral expression vector of claim 1, wherein the heterologous polypeptide comprises a CPI V/P gene that contains mutations at amino acid residue S157 or S308, or the combination thereof, wherein serine (S) is substituted with an amino acid residue selected from a group consisting of alanine (A), asparagine (B), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), selenocysteine (U), valine (V), tryptophan (W), tyrosine (Y), and glutamine (Z).

12. The viral expression vector of claim 11, wherein the amino acid substitution at amino acid residue S157 comprises a substitution of serine (S) to phenylalanine (F) and the amino acid substitution at amino acid residue S308 comprises a substitution of serine (S) to alanine (A) or Glycine (G).

13. The viral expression vector of claim 1, wherein a viral particle comprises the viral expression vector.

14. A composition comprising a viral expression vector comprising a parainfluenza virus 5 (PIV5) genome having a heterologous nucleic acid sequence with at least 98% sequence identity to SEQ ID NOs: 61, 62, 63, 64, 65, 66, 67, 68, 69, 70, 71, 72, 73, 74, 75, 76, 77, 78, 79, or 80, wherein the viral expression vector expresses a heterologous polypeptide comprising a coronavirus spike (S) and/or nucleocapsid (N) proteins.

15. The composition of claim 14, wherein a heterologous coronavirus spike (S) and nucleocapsid (N) proteins are expressed in a cell by contacting the cell with the composition.

16. A method of inducing an immune response in a subject having coronavirus disease 2019 (COVID-19), the method comprising administering a composition comprising a viral expression vector or a viral particle comprising a parainfluenza virus 5 (PIV5) genome having a heterologous nucleic acid sequence with at least 98% sequence identity to SEQ ID NOs: 61, 62, 63, 64, 65, 66, 67, 68, 69, 70, 71, 72, 73, 74, 75, 76, 77, 78, 79, or 80,
wherein the viral expression vector expresses a heterologous polypeptide comprising a coronavirus spike (S) and/or nucleocapsid (N) proteins, and
wherein the immune response comprises a humoral immune response and/or a cellular immune response.

17. The method of claim 16, wherein the subject is vaccinated against COVID-19), the method comprising administering the composition to the subject.

18. The method of claim 16, wherein the composition is administered intranasally, intramuscularly, topically, or orally.

19. The method of claim 16, further inducing in a subject an immune response comprising administering a PIV-5 booster vaccine composition comprising a viral expression vector or a viral particle having a PIV5 genome comprising a heterologous nucleic acid sequence with at least 98% sequence identity to SEQ ID NOs: 61, 62, 63, 64, 65, 66, 67, 68, 69, 70, 71, 72, 73, 74, 75, 76, 77, 78, 79, or 80, wherein said subject has previously received a primary vaccination against SARS-COV-2.


SEQUENCE LISTING (80 sequences)

SEQ ID NO: 1 - DNA, 35 bp, synthetic construct
  misc_feature 1..35: note = Synthetic: oCVL101
  source 1..35: mol_type = other DNA
acattaatggcatcaatcagttctatttctcaatt

SEQ ID NO: 2 - DNA, 30 bp, synthetic construct
  misc_feature 1..30: note = Synthetic: oCVL106
  source 1..30: mol_type = other DNA
agattagaacatgatgattgggtacattct

SEQ ID NO: 3 - DNA, 30 bp, synthetic construct
  misc_feature 1..30: note = Synthetic: oCVL119
  source 1..30: mol_type = other DNA
cggtgccaacaacacaatccacaatctaca

SEQ ID NO: 4 - DNA, 30 bp, synthetic construct
  misc_feature 1..30: note = Synthetic: oCVL120
  source 1..30: mol_type = other DNA
gctattgttagagtaggccacgctgttctc

SEQ ID NO: 5 - DNA, 30 bp, synthetic construct
  misc_feature 1..30: note = Synthetic: oCVL121
  source 1..30: mol_type = other DNA
gagaacagcgtggcctactctaacaatagc

SEQ ID NO: 6 - DNA, 30 bp, synthetic construct
  misc_feature 1..30: note = Synthetic: oCVL122
  source 1..30: mol_type = other DNA
atatgatgtgagtaggaagtatggcaggag

SEQ ID NO: 7 - DNA, 30 bp, synthetic construct; identical to SEQ ID NO: 3
  misc_feature 1..30: note = Synthetic: oCVL119
  source 1..30: mol_type = other DNA

SEQ ID NO: 8 - DNA, 30 bp, synthetic construct
  misc_feature 1..30: note = Synthetic: oCVL127
  source 1..30: mol_type = other DNA
gtgaattgtaccgaggtgcccgtggctatc

SEQ ID NO: 9 - DNA, 30 bp, synthetic construct
  misc_feature 1..30: note = Synthetic: oCVL128
  source 1..30: mol_type = other DNA
gatagccacgggcacctcggtacaattcac

SEQ ID NO: 10 - DNA, 30 bp, synthetic construct; identical to SEQ ID NO: 6
  misc_feature 1..30: note = Synthetic: oCVL122
  source 1..30: mol_type = other DNA

SEQ ID NO: 11 - DNA, 23 bp, synthetic construct
  misc_feature 1..23: note = Synthetic: oCVL17
  source 1..23: mol_type = other DNA
caagatgcaccttctctccagtg

SEQ ID NO: 12 - DNA, 59 bp, synthetic construct
  misc_feature 1..59: note = Synthetic: oCVL132
  source 1..59: mol_type = other DNA
gttctgtggtccgttatctgacatggtggcgcgcgatttgagctttccgagacggtagc

SEQ ID NO: 13 - DNA, 34 bp, synthetic construct
  misc_feature 1..34: note = Synthetic: oCVL133
  source 1..34: mol_type = other DNA
gcgcgccaccatgtcagataacggaccacagaac

SEQ ID NO: 14 - DNA, 25 bp, synthetic construct
  misc_feature 1..25: note = Synthetic: oCVL134
  source 1..25: mol_type = other DNA
tcaagcctgggtggagtcggcactg

SEQ ID NO: 15 - DNA, 57 bp, synthetic construct
  misc_feature 1..57: note = Synthetic: oCVL135
  source 1..57: mol_type = other DNA
cagtgccgactccacccaggcttgacgtacgacctgctataggctatccactgcatc

SEQ ID NO: 16 - DNA, 20 bp, synthetic construct
  misc_feature 1..20: note = Synthetic: oCVL124
  source 1..20: mol_type = other DNA
caggccgttgaaattgaagt

SEQ ID NO: 17 - DNA, 23 bp, synthetic construct
  misc_feature 1..23: note = Synthetic: oCVL17
  source 1..23: mol_type = other DNA
caagatgcaccttctctccagtg

SEQ ID NO: 18 - DNA, 59 bp, synthetic construct; identical to SEQ ID NO: 12
  misc_feature 1..59: note = Synthetic: oCVL132
  source 1..59: mol_type = other DNA

SEQ ID NO: 19 - DNA, 34 bp, synthetic construct; identical to SEQ ID NO: 13
  misc_feature 1..34: note = Synthetic: oCVL133
  source 1..34: mol_type = other DNA

SEQ ID NO: 20 - DNA, 58 bp, synthetic construct
  misc_feature 1..58: note = Synthetic: oCVL136
  source 1..58: mol_type = other DNA
gatatgatgtgagtaggaagtatggcaggaggctagctcaagcctgggtggagtcggc

SEQ ID NO: 21 - DNA, 35 bp, synthetic construct; identical to SEQ ID NO: 1
  misc_feature 1..35: note = Synthetic: oCVL101
  source 1..35: mol_type = other DNA

SEQ ID NO: 22 - DNA, 30 bp, synthetic construct
  misc_feature 1..30: note = Synthetic: oCVL137
  source 1..30: mol_type = other DNA
ttaggatagtgtcacctgacggataaatgg

SEQ ID NO: 23 - DNA, 53 bp, synthetic construct
  misc_feature 1..53: note = Synthetic: oCVL138
  source 1..53: mol_type = other DNA
ccatttatccgtcaggtgacactatcctaaacctgctataggctatccactgc

SEQ ID NO: 24 - DNA, 25 bp, synthetic construct
  misc_feature 1..25: note = Synthetic: oCVL139
  source 1..25: mol_type = other DNA
tcatttatgataaacaaaattctcc

SEQ ID NO: 25 - DNA, 60 bp, synthetic construct
  misc_feature 1..60: note = Synthetic: oCVL140
  source 1..60: mol_type = other DNA
ggagaattttgtttatcataaatgacgtacgaggcagaagcctccaggtctgacccagcc

SEQ ID NO: 26 - DNA, 30 bp, synthetic construct; identical to SEQ ID NO: 2
  misc_feature 1..30: note = Synthetic: oCVL106
  source 1..30: mol_type = other DNA

SEQ ID NO: 27 - DNA, 21 bp, synthetic construct
  misc_feature 1..21: note = Synthetic: oCVL141
  source 1..21: mol_type = other DNA
gcaagatcagagtgaggaagg

SEQ ID NO: 28 - DNA, 26 bp, synthetic construct
  misc_feature 1..26: note = Synthetic: oCVL142
  source 1..26: mol_type = other DNA
aattggttgcgataggattctctctg

SEQ ID NO: 29 - DNA, 25 bp, synthetic construct
  misc_feature 1..25: note = Synthetic: oCVL143
  source 1..25: mol_type = other DNA
ccccatcgattttaagaggggcagg

SEQ ID NO: 30 - DNA, 27 bp, synthetic construct
  misc_feature 1..27: note = Synthetic: oCVL144
  source 1..27: mol_type = other DNA
ggcatcattgaatgattctggcacaac

SEQ ID NO: 31 - DNA, 25 bp, synthetic construct
  misc_feature 1..25: note = Synthetic: oCVL145
  source 1..25: mol_type = other DNA
ttcttgactcaatctgaagatgtaa

SEQ ID NO: 32 - DNA, 20 bp, synthetic construct
  misc_feature 1..20: note = Synthetic: oCVL146
  source 1..20: mol_type = other DNA
gctgtaggatcacagcagca

SEQ ID NO: 33 - DNA, 23 bp, synthetic construct
  misc_feature 1..23: note = Synthetic: oCVL17
  source 1..23: mol_type = other DNA
caagatgcaccttctctccagtg

SEQ ID NO: 34 - DNA, 35 bp, synthetic construct
  misc_feature 1..35: note = Synthetic: oCVL159
  source 1..35: mol_type = other DNA
cgtacgttagcgcgcgatttgagctttccgagacg

SEQ ID NO: 35 - DNA, 60 bp, synthetic construct
  misc_feature 1..60: note = Synthetic: oCVL160
  source 1..60: mol_type = other DNA
tctcggaaagctcaaatcgcgcgctaacgtacgcacctgctataggctatccactgcatc

SEQ ID NO: 36 - DNA, 20 bp, synthetic construct
  misc_feature 1..20: note = Synthetic: oCVL161
  source 1..20: mol_type = other DNA
aaactgcaaggggtgatgtc

SEQ ID NO: 37 - DNA, 30 bp, synthetic construct; identical to SEQ ID NO: 3
  misc_feature 1..30: note = Synthetic: oCVL119
  source 1..30: mol_type = other DNA

SEQ ID NO: 38 - DNA, 60 bp, synthetic construct
  misc_feature 1..60: note = Synthetic: oCVL262
  source 1..60: mol_type = other DNA
ggctgggtcagacctggaggcttctgcctcgtacgaattcatttatgataaacaaaattc

SEQ ID NO: 39 - DNA, 23 bp, synthetic construct
  misc_feature 1..23: note = Synthetic: oCVL422
  source 1..23: mol_type = other DNA
aacacaatccacaatctacagtc

SEQ ID NO: 40 - DNA, 26 bp, synthetic construct
  misc_feature 1..26: note = Synthetic: oCVL423
  source 1..26: mol_type = other DNA
ggtcaggccgttgaagttgaagttca

SEQ ID NO: 41 - DNA, 23 bp, synthetic construct
  misc_feature 1..23: note = Synthetic: oCVL422
  source 1..23: mol_type = other DNA
aacacaatccacaatctacagtc

SEQ ID NO: 42 - DNA, 25 bp, synthetic construct
  misc_feature 1..25: note = Synthetic: oCVL611
  source 1..25: mol_type = other DNA
gatggcgtggaaccaggtcacgttg

SEQ ID NO: 43 - DNA, 48 bp, synthetic construct
  misc_feature 1..48: note = Synthetic: oCVL612
  source 1..48: mol_type = other DNA
caacgtgacctggttccacgccatcagcggcaccaatggcacaaagcg

SEQ ID NO: 44 - DNA, 90 bp, synthetic construct
  misc_feature 1..90: note = Synthetic: oCVL613
  source 1..90: mol_type = other DNA
cggcctggtagatttctgtagagatgtccctctcgaagggcttcagattgctctttctaaacagccggtatctataattgtagttgccgc

SEQ ID NO: 45 - DNA, 90 bp, synthetic construct
  misc_feature 1..90: note = Synthetic: oCVL614
  source 1..90: mol_type = other DNA
tacagaaatctaccaggccggcaataagccttgcaatggcgtggccggcgtgaactgttatttcccactccagtcctacggcttccgccc

SEQ ID NO: 46 - DNA, 30 bp, synthetic construct; identical to SEQ ID NO: 6
  misc_feature 1..30: note = Synthetic: oCVL122
  source 1..30: mol_type = other DNA

SEQ ID NO: 47 - DNA, 23 bp, synthetic construct
  misc_feature 1..23: note = Synthetic: oCVL422
  source 1..23: mol_type = other DNA
aacacaatccacaatctacagtc

SEQ ID NO: 48 - DNA, 25 bp, synthetic construct
  misc_feature 1..25: note = Synthetic: oCVL618
  source 1..25: mol_type = other DNA
tcccttcctggacgtgtactatcac

SEQ ID NO: 49 - DNA, 25 bp, synthetic construct
  misc_feature 1..25: note = Synthetic: oCVL619
  source 1..25: mol_type = other DNA
catatgtggggcggaagccgtagga

SEQ ID NO: 50 - DNA, 25 bp, synthetic construct
  misc_feature 1..25: note = Synthetic: oCVL620
  source 1..25: mol_type = other DNA
gtgatagtacacgtccaggaaggga

SEQ ID NO: 51 - DNA, 23 bp, synthetic construct
  misc_feature 1..23: note = Synthetic: oCVL621
  source 1..23: mol_type = other DNA
cagtcctacggcttccgccccac

SEQ ID NO: 52 - DNA, 30 bp, synthetic construct; identical to SEQ ID NO: 6
  misc_feature 1..30: note = Synthetic: oCVL122
  source 1..30: mol_type = other DNA

SEQ ID NO: 53 - DNA, 25 bp, synthetic construct
  misc_feature 1..25: note = Synthetic: oCVL5
  source 1..25: mol_type = other DNA
acagtggattccataaggttcctgc

SEQ ID NO: 54 - DNA, 24 bp, synthetic construct
  misc_feature 1..24: note = Synthetic: oCVL6
  source 1..24: mol_type = other DNA
ggagaagctcagatcagtgggatc

SEQ ID NO: 55 - DNA, 24 bp, synthetic construct
  misc_feature 1..24: note = Synthetic: oCVL622
  source 1..24: mol_type = other DNA
agctcattccagctccgtcaggtt

SEQ ID NO: 56 - DNA, 53 bp, synthetic construct
  misc_feature 1..53: note = Synthetic: oCVL624
  source 1..53: mol_type = other DNA
aacctgacggagctggaatgagctagccctgctataggctatccactgcatca

SEQ ID NO: 57 - DNA, 19 bp, synthetic construct
  misc_feature 1..19: note = Synthetic: oCVL625
  source 1..19: mol_type = other DNA
cgataggcaggtccaccag

SEQ ID NO: 58 - DNA, 58 bp, synthetic construct
  misc_feature 1..58: note = Synthetic: oCVL626
  source 1..58: mol_type = other DNA
ggtcagacctggaggcttctgcctcgtacgtcatcatttatgataaacaaaattctcc

SEQ ID NO: 59 - DNA, 30 bp, synthetic construct; identical to SEQ ID NO: 3
  misc_feature 1..30: note = Synthetic: oCVL119
  source 1..30: mol_type = other DNA

SEQ ID NO: 60 - DNA, 21 bp, synthetic construct
  misc_feature 1..21: note = Synthetic: oCVL166
  source 1..21: mol_type = other DNA
accagggggaaaacgaagtgg

SEQ ID NO: 61 - DNA, 20550 bp, synthetic construct
  misc_feature 1..20550: note = Synthetic: nucleic acid sequence for CVL34
  source 1..20550: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaacgcgtgctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagccttcaggtctgacccagccaatcaaagcattataccagaccatggcctaccatcggctttaaagaaaaaaataggcccggacgggttagcaacaagcggcggccgcgaattcgccaccatgtcagataacggaccacagaaccagaggaacgcacccaggattactttcggaggaccaagcgatagcaccgggagcaaccagaatggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaacaaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggaagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgtctagaatcggcatggaggtgacccccagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctccaaacagctgcagcagagcatgtccagtgccgactccacccaggcttgatgatgaggtacctgcggccgcctaaggtcgactcatggaatgcataccaaacattattgacactaatgacacacaaaattggttttaagaaaaaccaagagaacaataggccagaatgatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 62 - DNA, 19170 bp, synthetic construct
  misc_feature 1..19170: note = Synthetic: nucleic acid sequence for CVL44
  source 1..19170: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccaccatgttcgtgttcctggtgctgctgccactggtcagcagccagtgcgtgaatttcactacccggacccagctgcctcctgcttatacaaacagcttcaccaggggagtgtactaccccgataaggtgttccgcagctctgtgctgcacagcacccaggacttgtttcttcccttcttcagcaacgtgacctggttccacgccatccacgtgtccggcaccaacggcaccaaaagattcgccaatcctgtgctgcctttcaacgatggcgtgtactttgcctctacagagaagtctaacatcatcagaggctggatcttcggcacaaccctggattctaaaacccagagcctgctgatcgtgaacaatgccaccaatgtagtgatcaaggtttgtgagttccagttctgcaacgacccctttctgggcgtttactaccacaagaacaacaaaagctggatggaaagcgagttcagggtgtactctagcgccaacaactgtaccttcgagtacgtgagccagccttttctgatggacctggagggcaagcagggcaacttcaaaaacctgcgggagttcgtgtttaagaacatcgacggatacttcaagatttactccaagcacaccccaattaacttggttagaggactgccccaaggcttcagcgccctggaacccctggtggacctgcctatcggcatcaacatcacaagattccaaaccctgcatagaagctatctgacaccaggcgactctagctctggatggacagccggcgccgctgcttattacgtgggctacctgcagcctagaaccttcctcctgaaatacaacgagaacggaaccatcaccgacgccgtggactgcgccctggatcctctgagcgagacaaagtgcaccctgaagtccttcaccgtcgaaaagggcatctaccaaacaagcaacttcagagtgcagcctactgagagcatcgtgcggtttccaaacatcaccaacctgtgtccttttggcgaggtgttcaacgccacccggttcgcttccgtgtacgcctggaatagaaagcggatcagcaactgcgtggccgactacagcgtgctgtataatagcgcctcattttctacttttaaatgttacggcgtgtctcctacaaagctgaacgatctgtgcttcacaaatgtgtacgccgatagcttcgtgattagaggcgacgaggtgcggcagatcgcccctggccagaccggcaacatcgctgactacaactacaagctgcctgacgacttcaccggctgtgtgatcgcatggaacagcaataacctggactctaaggtgggcggaaactacaattacctgtacagactgttcagaaagagcaacctgaagcccttcgagagagatatctccacagaaatctatcaggccggatctacaccttgtaatggcgtgaagggcttcaactgctacttccctctgcaatcctacggctttcagcccacatacggcgtgggctaccagccataccgggtcgtggtgctgagcttcgagctgctgcacgcccctgccacagtgtgcggccctaagaagagcaccaatctggtaaagaacaagtgcgtcaacttcaatttcaacggcctgaccgggaccggagtgctgaccgaaagcaacaagaaattcctgccgttccagcagttcggcagagatatcgccgacaccaccgatgccgtgcgggacccccagacacttgaaatcctggacatcaccccttgcagttttggcggcgtgtccgtgatcacacctggaacaaacaccagcaaccaggtggccgtgctgtaccagggcgtcaactgcaccgaggtccccgtggccatccacgccgatcagctgacacctacatggcgggtgtactctaccggcagcaacgtgttccaaaccagagccggctgcctgatcggcgctgagcacgtgaacaacagctacgagtgcgacattcctatcggtgctggcatctgcgcctcttaccagacacagacaaacagccccagaagagcgagaagcgtggctagtcagagcatcatcgcctacaccatgagcctgggcgtggagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaacgcgtgctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 63 - DNA, 19164 bp, synthetic construct
  misc_feature 1..19164: note = Synthetic: nucleic acid sequence for CVL48
  source 1..19164: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccaccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatcagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggatcccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacatatggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgatgataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcacagaagagcccggagcgtggcctcccagtctatcatcgcctataccatgtccctgggcgccgagaacagcgtggcctactctaacaatagcatcgccatcccaatcaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctggctcggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacacacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgagctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 64 - DNA, 19170 bp, synthetic construct
  misc_feature 1..19170: note = Synthetic: nucleic acid sequence for CVL49
  source 1..19170: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccatgttcgtcttcctggtcctgctgcctctggtctcctcacagtgcgtcaatttcacaaaccggactcagctgccatctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaattaccccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagcgagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggatcccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcacgatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtgaagggctttaactgttatttcccactccagtcctacggcttccagcccacatacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagtacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaattaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctcctttgtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgagctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 65 - DNA, 19176 bp, synthetic construct
  misc_feature 1..19176: note = Synthetic: nucleic acid sequence for CVL50
  source 1..19176: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccatgttcgtcttcctggtcctgctgcctctggtctccatccagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctgcatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggatcccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatcggtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaacgcgtgctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 66 - DNA, 20310 bp, synthetic construct
  misc_feature 1..20310: note = Synthetic: nucleic acid sequence for CVL52
  source 1..20310: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcgcgcgccaccatgtcagataacggaccacagaaccagaggaacgcacccaggattactttcggaggaccaagcgatagcaccgggagcaaccagaatggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaacaaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggaagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgtctagaatcggcatggaggtgacccccagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctccaaacagctgcagcagagcatgtccagtgccgactccacccaggcttgacgtacgacctgctataggctatccactgcatcatctctttttaagaaaaaaataggcccggacgggttagcaacaagcgactgccggtgccaacaacacaatccacaatctacagtcgacgcggccgccaccatgttcgtgttcctggtgctgctgccactggtcagcagccagtgcgtgaatttcactacccggacccagctgcctcctgcttatacaaacagcttcaccaggggagtgtactaccccgataaggtgttccgcagctctgtgctgcacagcacccaggacttgtttcttcccttcttcagcaacgtgacctggttccacgccatccacgtgtccggcaccaacggcaccaaaagattcgccaatcctgtgctgcctttcaacgatggcgtgtactttgcctctacagagaagtctaacatcatcagaggctggatcttcggcacaaccctggattctaaaacccagagcctgctgatcgtgaacaatgccaccaatgtagtgatcaaggtttgtgagttccagttctgcaacgacccctttctgggcgtttactaccacaagaacaacaaaagctggatggaaagcgagttcagggtgtactctagcgccaacaactgtaccttcgagtacgtgagccagccttttctgatggacctggagggcaagcagggcaacttcaaaaacctgcgggagttcgtgtttaagaacatcgacggatacttcaagatttactccaagcacaccccaattaacttggttagaggactgccccaaggcttcagcgccctggaacccctggtggacctgcctatcggcatcaacatcacaagattccaaaccctgcatagaagctatctgacaccaggcgactctagctctggatggacagccggcgccgctgcttattacgtgggctacctgcagcctagaaccttcctcctgaaatacaacgagaacggaaccatcaccgacgccgtggactgcgccctggatcctctgagcgagacaaagtgcaccctgaagtccttcaccgtcgaaaagggcatctaccaaacaagcaacttcagagtgcagcctactgagagcatcgtgcggtttccaaacatcaccaacctgtgtccttttggcgaggtgttcaacgccacccggttcgcttccgtgtacgcctggaatagaaagcggatcagcaactgcgtggccgactacagcgtgctgtataatagcgcctcattttctacttttaaatgttacggcgtgtctcctacaaagctgaacgatctgtgcttcacaaatgtgtacgccgatagcttcgtgattagaggcgacgaggtgcggcagatcgcccctggccagaccggcaacatcgctgactacaactacaagctgcctgacgacttcaccggctgtgtgatcgcatggaacagcaataacctggactctaaggtgggcggaaactacaattacctgtacagactgttcagaaagagcaacctgaagcccttcgagagagatatctccacagaaatctatcaggccggatctacaccttgtaatggcgtgaagggcttcaactgctacttccctctgcaatcctacggctttcagcccacatacggcgtgggctaccagccataccgggtcgtggtgctgagcttcgagctgctgcacgcccctgccacagtgtgcggccctaagaagagcaccaatctggtaaagaacaagtgcgtcaacttcaatttcaacggcctgaccgggaccggagtgctgaccgaaagcaacaagaaattcctgccgttccagcagttcggcagagatatcgccgacaccaccgatgccgtgcgggacccccagacacttgaaatcctggacatcaccccttgcagttttggcggcgtgtccgtgatcacacctggaacaaacaccagcaaccaggtggccgtgctgtaccagggcgtcaactgcaccgaggtccccgtggccatccacgccgatcagctgacacctacatggcgggtgtactctaccggcagcaacgtgttccaaaccagagccggctgcctgatcggcgctgagcacgtgaacaacagctacgagtgcgacattcctatcggtgctggcatctgcgcctcttaccagacacagacaaacagccccagaagagcgagaagcgtggctagtcagagcatcatcgcctacaccatgagcctgggcgtggagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaacgcgtgctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 67 - DNA, 16356 bp, synthetic construct
  misc_feature 1..16356: note = Synthetic: nucleic acid sequence for CVL53
  source 1..16356: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcgcgcgccaccatgtcagataacggaccacagaaccagaggaacgcacccaggattactttcggaggaccaagcgatagcaccgggagcaaccagaatggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaacaaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggaagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgtctagaatcggcatggaggtgacccccagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctccaaacagctgcagcagagcatgtccagtgccgactccacccaggcttgagctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 68 - DNA, 20304 bp, synthetic construct
  misc_feature 1..20304: note = Synthetic: nucleic acid sequence for CVL54
  source 1..20304: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcgcgcgccaccatgtcagataacggaccacagaaccagaggaacgcacccaggattactttcggaggaccaagcgatagcaccgggagcaaccagaatggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaacaaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggaagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgtctagaatcggcatggaggtgacccccagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctccaaacagctgcagcagagcatgtccagtgccgactccacccaggcttgagctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaacctgctataggctatccactgcatcatctctttttaagaaaaaaataggcccggacgggttagcaacaagcgactgccggtgccaacaacacaatccacaatctacagtcgacgcggccgccaccatgttcgtgttcctggtgctgctgccactggtcagcagccagtgcgtgaatttcactacccggacccagctgcctcctgcttatacaaacagcttcaccaggggagtgtactaccccgataaggtgttccgcagctctgtgctgcacagcacccaggacttgtttcttcccttcttcagcaacgtgacctggttccacgccatccacgtgtccggcaccaacggcaccaaaagattcgccaatcctgtgctgcctttcaacgatggcgtgtactttgcctctacagagaagtctaacatcatcagaggctggatcttcggcacaaccctggattctaaaacccagagcctgctgatcgtgaacaatgccaccaatgtagtgatcaaggtttgtgagttccagttctgcaacgacccctttctgggcgtttactaccacaagaacaacaaaagctggatggaaagcgagttcagggtgtactctagcgccaacaactgtaccttcgagtacgtgagccagccttttctgatggacctggagggcaagcagggcaacttcaaaaacctgcgggagttcgtgtttaagaacatcgacggatacttcaagatttactccaagcacaccccaattaacttggttagaggactgccccaaggcttcagcgccctggaacccctggtggacctgcctatcggcatcaacatcacaagattccaaaccctgcatagaagctatctgacaccaggcgactctagctctggatggacagccggcgccgctgcttattacgtgggctacctgcagcctagaaccttcctcctgaaatacaacgagaacggaaccatcaccgacgccgtggactgcgccctggatcctctgagcgagacaaagtgcaccctgaagtccttcaccgtcgaaaagggcatctaccaaacaagcaacttcagagtgcagcctactgagagcatcgtgcggtttccaaacatcaccaacctgtgtccttttggcgaggtgttcaacgccacccggttcgcttccgtgtacgcctggaatagaaagcggatcagcaactgcgtggccgactacagcgtgctgtataatagcgcctcattttctacttttaaatgttacggcgtgtctcctacaaagctgaacgatctgtgcttcacaaatgtgtacgccgatagcttcgtgattagaggcgacgaggtgcggcagatcgcccctggccagaccggcaacatcgctgactacaactacaagctgcctgacgacttcaccggctgtgtgatcgcatggaacagcaataacctggactctaaggtgggcggaaactacaattacctgtacagactgttcagaaagagcaacctgaagcccttcgagagagatatctccacagaaatctatcaggccggatctacaccttgtaatggcgtgaagggcttcaactgctacttccctctgcaatcctacggctttcagcccacatacggcgtgggctaccagccataccgggtcgtggtgctgagcttcgagctgctgcacgcccctgccacagtgtgcggccctaagaagagcaccaatctggtaaagaacaagtgcgtcaacttcaatttcaacggcctgaccgggaccggagtgctgaccgaaagcaacaagaaattcctgccgttccagcagttcggcagagatatcgccgacaccaccgatgccgtgcgggacccccagacacttgaaatcctggacatcaccccttgcagttttggcggcgtgtccgtgatcacacctggaacaaacaccagcaaccaggtggccgtgctgtaccagggcgtcaactgcaccgaggtccccgtggccatccacgccgatcagctgacacctacatggcgggtgtactctaccggcagcaacgtgttccaaaccagagccggctgcctgatcggcgctgagcacgtgaacaacagctacgagtgcgacattcctatcggtgctggcatctgcgcctcttaccagacacagacaaacagccccagaagagcgagaagcgtggctagtcagagcatcatcgcctacaccatgagcctgggcgtggagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgacgtacgaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 69 - DNA, 19170 bp, synthetic construct
  misc_feature 1..19170: note = Synthetic: nucleic acid sequence for CVL55
  source 1..19170: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaatttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatgccttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccaccatgttcgtgttcctggtgctgctgccactggtcagcagccagtgcgtgaatttcactacccggacccagctgcctcctgcttatacaaacagcttcaccaggggagtgtactaccccgataaggtgttccgcagctctgtgctgcacagcacccaggacttgtttcttcccttcttcagcaacgtgacctggttccacgccatccacgtgtccggcaccaacggcaccaaaagattcgccaatcctgtgctgcctttcaacgatggcgtgtactttgcctctacagagaagtctaacatcatcagaggctggatcttcggcacaaccctggattctaaaacccagagcctgctgatcgtgaacaatgccaccaatgtagtgatcaaggtttgtgagttccagttctgcaacgacccctttctgggcgtttactaccacaagaacaacaaaagctggatggaaagcgagttcagggtgtactctagcgccaacaactgtaccttcgagtacgtgagccagccttttctgatggacctggagggcaagcagggcaacttcaaaaacctgcgggagttcgtgtttaagaacatcgacggatacttcaagatttactccaagcacaccccaattaacttggttagaggactgccccaaggcttcagcgccctggaacccctggtggacctgcctatcggcatcaacatcacaagattccaaaccctgcatagaagctatctgacaccaggcgactctagctctggatggacagccggcgccgctgcttattacgtgggctacctgcagcctagaaccttcctcctgaaatacaacgagaacggaaccatcaccgacgccgtggactgcgccctggatcctctgagcgagacaaagtgcaccctgaagtccttcaccgtcgaaaagggcatctaccaaacaagcaacttcagagtgcagcctactgagagcatcgtgcggtttccaaacatcaccaacctgtgtccttttggcgaggtgttcaacgccacccggttcgcttccgtgtacgcctggaatagaaagcggatcagcaactgcgtggccgactacagcgtgctgtataatagcgcctcattttctacttttaaatgttacggcgtgtctcctacaaagctgaacgatctgtgcttcacaaatgtgtacgccgatagcttcgtgattagaggcgacgaggtgcggcagatcgcccctggccagaccggcaacatcgctgactacaactacaagctgcctgacgacttcaccggctgtgtgatcgcatggaacagcaataacctggactctaaggtgggcggaaactacaattacctgtacagactgttcagaaagagcaacctgaagcccttcgagagagatatctccacagaaatctatcaggccggatctacaccttgtaatggcgtgaagggcttcaactgctacttccctctgcaatcctacggctttcagcccacatacggcgtgggctaccagccataccgggtcgtggtgctgagcttcgagctgctgcacgcccctgccacagtgtgcggccctaagaagagcaccaatctggtaaagaacaagtgcgtcaacttcaatttcaacggcctgaccgggaccggagtgctgaccgaaagcaacaagaaattcctgccgttccagcagttcggcagagatatcgccgacaccaccgatgccgtgcgggacccccagacacttgaaatcctggacatcaccccttgcagttttggcggcgtgtccgtgatcacacctggaacaaacaccagcaaccaggtggccgtgctgtaccagggcgtcaactgcaccgaggtccccgtggccatccacgccgatcagctgacacctacatggcgggtgtactctaccggcagcaacgtgttccaaaccagagccggctgcctgatcggcgctgagcacgtgaacaacagctacgagtgcgacattcctatcggtgctggcatctgcgcctcttaccagacacagacaaacagccccagaagagcgagaagcgtggctagtcagagcatcatcgcctacaccatgagcctgggcgtggagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaacgcgtgctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 70 - DNA, 19050 bp, synthetic construct
  misc_feature 1..19050: note = Synthetic: nucleic acid sequence for CVL58
  source 1..19050: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcgcgcgctaacgtacgcacctgctataggctatccactgcatcatctctttttaagaaaaaaataggcccggacgggttagcaacaagcgactgccggtgccaacaacacaatccacaatctacagtcgacgcggccgccaccatgttcgtgttcctggtgctgctgccactggtcagcagccagtgcgtgaatttcactacccggacccagctgcctcctgcttatacaaacagcttcaccaggggagtgtactaccccgataaggtgttccgcagctctgtgctgcacagcacccaggacttgtttcttcccttcttcagcaacgtgacctggttccacgccatccacgtgtccggcaccaacggcaccaaaagattcgccaatcctgtgctgcctttcaacgatggcgtgtactttgcctctacagagaagtctaacatcatcagaggctggatcttcggcacaaccctggattctaaaacccagagcctgctgatcgtgaacaatgccaccaatgtagtgatcaaggtttgtgagttccagttctgcaacgacccctttctgggcgtttactaccacaagaacaacaaaagctggatggaaagcgagttcagggtgtactctagcgccaacaactgtaccttcgagtacgtgagccagccttttctgatggacctggagggcaagcagggcaacttcaaaaacctgcgggagttcgtgtttaagaacatcgacggatacttcaagatttactccaagcacaccccaattaacttggttagaggactgccccaaggcttcagcgccctggaacccctggtggacctgcctatcggcatcaacatcacaagattccaaaccctgcatagaagctatctgacaccaggcgactctagctctggatggacagccggcgccgctgcttattacgtgggctacctgcagcctagaaccttcctcctgaaatacaacgagaacggaaccatcaccgacgccgtggactgcgccctggatcctctgagcgagacaaagtgcaccctgaagtccttcaccgtcgaaaagggcatctaccaaacaagcaacttcagagtgcagcctactgagagcatcgtgcggtttccaaacatcaccaacctgtgtccttttggcgaggtgttcaacgccacccggttcgcttccgtgtacgcctggaatagaaagcggatcagcaactgcgtggccgactacagcgtgctgtataatagcgcctcattttctacttttaaatgttacggcgtgtctcctacaaagctgaacgatctgtgcttcacaaatgtgtacgccgatagcttcgtgattagaggcgacgaggtgcggcagatcgcccctggccagaccggcaacatcgctgactacaactacaagctgcctgacgacttcaccggctgtgtgatcgcatggaacagcaataacctggactctaaggtgggcggaaactacaattacctgtacagactgttcagaaagagcaacctgaagcccttcgagagagatatctccacagaaatctatcaggccggatctacaccttgtaatggcgtgaagggcttcaactgctacttccctctgcaatcctacggctttcagcccacatacggcgtgggctaccagccataccgggtcgtggtgctgagcttcgagctgctgcacgcccctgccacagtgtgcggccctaagaagagcaccaatctggtaaagaacaagtgcgtcaacttcaatttcaacggcctgaccgggaccggagtgctgaccgaaagcaacaagaaattcctgccgttccagcagttcggcagagatatcgccgacaccaccgatgccgtgcgggacccccagacacttgaaatcctggacatcaccccttgcagttttggcggcgtgtccgtgatcacacctggaacaaacaccagcaaccaggtggccgtgctgtaccagggcgtcaactgcaccgaggtccccgtggccatccacgccgatcagctgacacctacatggcgggtgtactctaccggcagcaacgtgttccaaaccagagccggctgcctgatcggcgctgagcacgtgaacaacagctacgagtgcgacattcctatcggtgctggcatctgcgcctcttaccagacacagacaaacagccccagaagagcgagaagcgtggctagtcagagcatcatcgcctacaccatgagcctgggcgtggagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaacgcgtgctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 71 - DNA, 19050 bp, synthetic construct
  misc_feature 1..19050: note = Synthetic: nucleic acid sequence for CVL58
  source 1..19050: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaatttccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatgccttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcgcgcgctaacgtacgcacctgctataggctatccactgcatcatctctttttaagaaaaaaataggcccggacgggttagcaacaagcgactgccggtgccaacaacacaatccacaatctacagtcgacgcggccgccaccatgttcgtgttcctggtgctgctgccactggtcagcagccagtgcgtgaatttcactacccggacccagctgcctcctgcttatacaaacagcttcaccaggggagtgtactaccccgataaggtgttccgcagctctgtgctgcacagcacccaggacttgtttcttcccttcttcagcaacgtgacctggttccacgccatccacgtgtccggcaccaacggcaccaaaagattcgccaatcctgtgctgcctttcaacgatggcgtgtactttgcctctacagagaagtctaacatcatcagaggctggatcttcggcacaaccctggattctaaaacccagagcctgctgatcgtgaacaatgccaccaatgtagtgatcaaggtttgtgagttccagttctgcaacgacccctttctgggcgtttactaccacaagaacaacaaaagctggatggaaagcgagttcagggtgtactctagcgccaacaactgtaccttcgagtacgtgagccagccttttctgatggacctggagggcaagcagggcaacttcaaaaacctgcgggagttcgtgtttaagaacatcgacggatacttcaagatttactccaagcacaccccaattaacttggttagaggactgccccaaggcttcagcgccctggaacccctggtggacctgcctatcggcatcaacatcacaagattccaaaccctgcatagaagctatctgacaccaggcgactctagctctggatggacagccggcgccgctgcttattacgtgggctacctgcagcctagaaccttcctcctgaaatacaacgagaacggaaccatcaccgacgccgtggactgcgccctggatcctctgagcgagacaaagtgcaccctgaagtccttcaccgtcgaaaagggcatctaccaaacaagcaacttcagagtgcagcctactgagagcatcgtgcggtttccaaacatcaccaacctgtgtccttttggcgaggtgttcaacgccacccggttcgcttccgtgtacgcctggaatagaaagcggatcagcaactgcgtggccgactacagcgtgctgtataatagcgcctcattttctacttttaaatgttacggcgtgtctcctacaaagctgaacgatctgtgcttcacaaatgtgtacgccgatagcttcgtgattagaggcgacgaggtgcggcagatcgcccctggccagaccggcaacatcgctgactacaactacaagctgcctgacgacttcaccggctgtgtgatcgcatggaacagcaataacctggactctaaggtgggcggaaactacaattacctgtacagactgttcagaaagagcaacctgaagcccttcgagagagatatctccacagaaatctatcaggccggatctacaccttgtaatggcgtgaagggcttcaactgctacttccctctgcaatcctacggctttcagcccacatacggcgtgggctaccagccataccgggtcgtggtgctgagcttcgagctgctgcacgcccctgccacagtgtgcggccctaagaagagcaccaatctggtaaagaacaagtgcgtcaacttcaatttcaacggcctgaccgggaccggagtgctgaccgaaagcaacaagaaattcctgccgttccagcagttcggcagagatatcgccgacaccaccgatgccgtgcgggacccccagacacttgaaatcctggacatcaccccttgcagttttggcggcgtgtccgtgatcacacctggaacaaacaccagcaaccaggtggccgtgctgtaccagggcgtcaactgcaccgaggtccccgtggccatccacgccgatcagctgacacctacatggcgggtgtactctaccggcagcaacgtgttccaaaccagagccggctgcctgatcggcgctgagcacgtgaacaacagctacgagtgcgacattcctatcggtgctggcatctgcgcctcttaccagacacagacaaacagccccagaagagcgagaagcgtggctagtcagagcatcatcgcctacaccatgagcctgggcgtggagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaacgcgtgctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 72 - DNA, 19164 bp, synthetic construct
  misc_feature 1..19164: note = Synthetic: nucleic acid sequence for CVL60
  source 1..19164: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccatgttcgtcttcctggtcctgctgcctctggtctcctcacagtgcgtcaatctgagaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccggcgtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatagataccggctgtttagaaagagcaatctgaagcccttcgagagggacatctctacagaaatctaccaggccggcagcaagccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctagaagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccagaacgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgagctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 73 - DNA, 19158 bp, synthetic construct
  misc_feature 1..19158: note = Synthetic: nucleic acid sequence for CVL64
  source 1..19158: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatgtgataaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatcagtaccggctgtttagaaagagcaatctgaagcccttcgagagggacatctctacagaaatctaccaggccggcagcaccccttgcaatggcgtggagggctttaactgttattccccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaacgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgatgaacgcgtgctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 74 - DNA, 20310 bp, synthetic construct
  misc_feature 1..20310: note = Synthetic: nucleic acid sequence for CVL65
  source 1..20310: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcgcgcgccaccatgtcagataacggaccacagaaccagaggaacgcacccaggattactttcggaggaccaagcgatagcaccgggagcaaccagaatggagagcggagcggagcaagatccaagcagagacggccccagggcctgccaaacaataccgcatcctggttcaccgccctgacacagcacggcaaggaggacctgaagtttccaaggggacagggagtgcctatcaacaccaatagctcccctgacgatcagatcggctactataggagggcaacaaggagaatcaggggaggcgacggcaagatgaaggatctgagcccacgctggtacttctactatctgggaaccggacctgaggcaggcctgccatatggcgccaacaaggacggaatcatctgggtggcaaccgagggcgccctgaacacaccaaaggatcacatcggcacaagaaatcccgccaacaatgcagcaatcgtgctgcagctgccacagggaaccacactgcccaagggcttttacgcagagggctctcggggaggcagccaggcatctagcagatcctctagccggagcagaaactcctctaggaattccaccccaggaagctccaggggcacatcccctgcccgcatggcaggaaacggaggcgacgccgccctggccctgctgctgctggatcgcctgaatcagctggagtccaagatgtctggcaagggacagcagcagcagggacagaccgtgacaaagaagtccgccgccgaggcctctaagaagccaaggcagaagcgcaccgccacaaaggcctacaacgtgacccaggccttcggcaggcgcggaccagagcagacacagggcaattttggcgaccaggagctgatcaggcagggaaccgattataagcactggcctcagatcgcccagttcgccccatctgccagcgccttctttggcatgtctagaatcggcatggaggtgacccccagcggcacatggctgacctacacaggcgccatcaagctggacgataaggaccctaacttcaaggatcaggtcatcctgctgaacaagcacatcgacgcctataagacctttccccctacagagcccaagaaggacaagaagaagaaggccgatgagacacaggccctgcctcagaggcagaagaagcagcagaccgtgacactgctgccagccgccgatctggacgatttctccaaacagctgcagcagagcatgtccagtgccgactccacccaggcttgagctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaacctgctataggctatccactgcatcatctctttttaagaaaaaaataggcccggacgggttagcaacaagcgactgccggtgccaacaacacaatccacaatctacagtcgacgcggccgccaccatgttcgtcttcctggtcctgctgcctctggtctcctcacagtgcgtcaatctgagaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccggcgtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatagataccggctgtttagaaagagcaatctgaagcccttcgagagggacatctctacagaaatctaccaggccggcagcaagccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctagaagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccagaacgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgaattcgtacgaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 75 - DNA, 19182 bp, synthetic construct
  misc_feature 1..19182: note = Synthetic: nucleic acid sequence for CVL67
  source 1..19182: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctatcgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgacatccaaccacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaaccaagttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtgaagggcttcaactgctacttccctctgcaatcctacggctttcagcccacatacggcgtgggctaccagccataccgggtcgtggtgctgagcttcgagctgctgcacgcccctgccacagtgtgcggccctaagaagagcaccaatctggtaaagaacaagtgcgtcaacttcaatttcaacggcctgaccgggaccggagtgctgaccgaaagcaacaagaaattcctgccgttccagcagttcggcagagatatcgccgacaccaccgatgccgtgcgggacccccagacacttgaaatcctggacatcaccccttgcagttttggcggcgtgtccgtgatcacacctggaacaaacaccagcaaccaggtggccgtgctgtaccagggcgtcaactgcaccgaggtccccgtggccatccacgccgatcagctgacacctacatggcgggtgtactctaccggcagcaacgtgttccaaaccagagccggctgcctgatcggcgctgagcacgtgaacaacagctacgagtgcgacattcctatcggtgctggcatctgcgcctcttaccagacacagacaaacagccacagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccagaatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgatgaacgcgtgctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 76 - DNA, 19164 bp, synthetic construct
  misc_feature 1..19164: note = Synthetic: nucleic acid sequence for CVL80
  source 1..19164: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccaccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgtgatcagcggcaccaatggcacaaagcggttcgacaatcccgtgctgccttttaacgatggcgtgtacttcgcctctatcgagaagagcaacatcatcagaggctggatctttggcaccacactggactccaagacacagtctctgctgatcgtgaacaatgccaccaacgtggtcatcaaggtgtgcgagttccagttttgtaatgatcccttcctggatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcatcgtgcgcgagcctgaggacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttgacgaggtgttcaacgcaacccgcttcgccagcgtgtacgcctggaataggaagcggatcagcaactgcgtggccgactatagcgtgctgtacaacctggcccccttcttcacctttaagtgctatggcgtgtcccccacaaagctgaatgacctgtgctttaccaacgtctacgccgattctttcgtgatcaggggcgacgaggtgcgccagatcgcccccggccagacaggcaatatcgcagactacaattataagctgccagacgatttcaccggctgcgtgatcgcctggaacagcaacaagctggattccaaagtgtccggcaactacaattatctgtaccggctgtttagaaagagcaatctgaagcccttcgagagggacatctctacagaaatctaccaggccggcaataagccttgcaatggcgtggccggctttaactgttatttcccactcaagtcctactccttccgccccacatatggcgtgggccaccagccttaccgcgtggtggtgctgagctttgagctgctgcacgccccagcaacagtgtgcggccccaagaagtccaccaatctggtgaagaacaagtgcgtgaacttcaacttcaacggcctgaagggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagtacgtgaacaattcctatgagtgcgacatcccaatcggcgccggcatctgtgcctcttaccagacccagacaaagtctcacagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaagagagccctgacaggcatcgccgtggagcaggataagaacacacaggaggtgttcgcccaggtgaagcaaatctacaagaccccccctatcaagtactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaagggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcacaacgcccaggccctgaataccctggtgaagcagctgagcagcaagttcggcgccatctctagcgtgctgaatgacatctttagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgagctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 77 - DNA, 19164 bp, synthetic construct
  misc_feature 1..19164: note = Synthetic: nucleic acid sequence for CVL86
  source 1..19164: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccaccatgttcgtcttcctggtcctgctgcctctggtctcctcacagtgcgtcaatctgatcactcggactcagtcctatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctggacgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctgggccgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttgacgaggtgttcaacgcaacccgcttcgccagcgtgtacgcctggaataggaagcggatcagcaactgcgtggccgactatagcgtgctgtacaacctggcccccttcttcgcctttaagtgctatggcgtgtcccccacaaagctgaatgacctgtgctttaccaacgtctacgccgattctttcgtgatcaggggcaacgaggtgtcccagatcgcccccggccagacaggcaatatcgcagactacaattataagctgccagacgatttcaccggctgcgtgatcgcctggaacagcaacaagctggattccaaagtgggcggcaactacaattatctgtaccggctgtttagaaagagcaatctgaagcccttcgagagggacatctctacagaaatctaccaggccggcaataagccttgcaatggcgtggccggctttaactgttatttcccactccgctcctacggcttccgccccacatatggcgtgggccaccagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagtacgtgaacaattcctatgagtgcgacatcccaatcggcgccggcatctgtgcctcttaccagacccagacaaagtctcacagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaagagagccctgacaggcatcgccgtggagcaggataagaacacacaggaggtgttcgcccaggtgaagcaaatctacaagaccccccctatcaagtactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcacaacgcccaggccctgaataccctggtgaagcagctgagcagcaagttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgagctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 78 - DNA, 19158 bp, synthetic construct
  misc_feature 1..19158: note = Synthetic: nucleic acid sequence for CVL108
  source 1..19158: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccaccatgttcgtcttcctggtcctgctgcctctggtctcctcacagtgcgtcaatctgatcactcggactcagtcctatactaatagcttcaccagaggcgtgtactatcctgacaaggtgtttagaagctccgtgctgcactctacacaggatctgtttctgccattctttagcaacgtgacctggttccacgccatcagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctggacgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctgggccgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttgacgaggtgttcaacgcaacccgcttcgccagcgtgtacgcctggaataggaagcggatcagcaactgcgtggccgactatagcgtgctgtacaacctggcccccttcttcgcctttaagtgctatggcgtgtcccccacaaagctgaatgacctgtgctttaccaacgtctacgccgattctttcgtgatcaggggcaacgaggtgtcccagatcgcccccggccagacaggcaatatcgcagactacaattataagctgccagacgatttcaccggctgcgtgatcgcctggaacagcaacaagctggattccaaagtgggcggcaactacaattatagataccggctgtttagaaagagcaatctgaagcccttcgagagggacatctctacagaaatctaccaggccggcaataagccttgcaatggcgtggccggcgtgaactgttatttcccactccagtcctacggcttccgccccacatatggcgtgggccaccagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagtacgtgaacaattcctatgagtgcgacatcccaatcggcgccggcatctgtgcctcttaccagacccagacaaagtctcacagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaagagagccctgacaggcatcgccgtggagcaggataagaacacacaggaggtgttcgcccaggtgaagcaaatctacaagaccccccctatcaagtactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcacaacgcccaggccctgaataccctggtgaagcagctgagcagcaagttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgagctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 79 - DNA, 19164 bp, synthetic construct
  misc_feature 1..19164: note = Synthetic: nucleic acid sequence for CVL111
  source 1..19164: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgactgacccccaatcgagactacaccacctcaaactataggtgggtggtacctcagtgattaatctcgtaagcactgatcgtaggctacaacacactaatattatccagattagagagcttaattagctctgtattaataataacactactattccaataactggaatcaccagcttgatttatctccaaaatgattcaaagaaaacaaatcatattaagactatcctaagcacgaacccatatcgtccttcaaatcatgggtactataattcaatttctggtggtctcctgtctattggcaggagcaggcagtcttgatccagcagccctcatgcaaatcggtgtcattccaacaaatgtccggcaacttatgtattatactgaggcctcatcagcattcattgttgtgaagttaatgcctacaattgactcgccgattagtggatgtaatataacatcaatttcaagctataatgcaacagtgacaaaactcctacagccgatcggtgagaatttggagacgattaggaaccagttgattccaactcggaggagacgccggtttgcaggggtggtgattggattagctgcattaggagtagctactgccgcacaggtcactgccgcagtagcactagtaaaggcaaatgaaaatgctgcggctatactcaatctcaaaaatgcaatccaaaaaacaaatgcagcagttgcagatgtggtccaggccacacaatcactaggaacagcagttcaagcagttcaagatcacataaacagtgtggtaagtccagcaattacagcagccaattgtaaagcccaagatgctatcattggctcaatcctcaatctctatttgaccgagttgacaactatcttccacaatcaaattacaaaccctgcattgagtcctattacaattcaagctttaaggatcctactagggagtaccttgccgactgtggtcgaaaaatctttcaatacccagataagtgcggctgagcttctctcatcagggttattgacaggccagattgtgggattagatttgacctatatgcagatggtcataaaaattgagctgccaactttaactgtacaacctgcaacccagatcatagatctggccaccatttctgcattcattaacaatcaagaagtcatggcccaattaccaacacgtgttattgtgactggcagcttgatccaagcctatcccgcatcgcaatgcactattacacccaacactgtgtactgtaggtataatgatgcccaagtactctcagatgatacgatggcttgcctccaaggtaacttgacaagatgcaccttctctccagtggttgggagctttctcactcgattcgtgctgttcgatggaatagtttatgcaaattgcaggtcgatgttgtgcaagtgcatgcagcctgctgctgtgatcctacagccgagttcatcccctgtaactgtcattgacatgtacaaatgtgtgagtctgcagcttgacaatctcagattcaccatcactcaattggccaatgtaacctacaatagcaccatcaagcttgaaacatcccagatcttgcctattgatccgttggatatatcccagaatctagctgcggtgaataagagtctaagtgatgcactacaacacttagcacaaagtgacacatacctttctgcaatcacatcagctacgactacaagtgtattatccataatagcaatctgtcttggatcgttaggtttaatattaataatcttgctcagtgtagttgtgtggaagttattgaccattgtcgctgctaatcgaaatagaatggagaattttgtttatcataattcagcattccaccactcacgatctgatctcagtgagaaaaatcaacctgcaactcttggaacaagataagacagtcatccattagtaatttttaagaaaaaaacgataggaccgaaactagtattgaaagaaccgtctcggtcaatctaggtaatcgagctgctaccgtctcggaaagctcaaatcatgctgcctgatccggaagatccggaaagcaaaaaagctacaaggagaacaggaaacctaattatctgcttcctattcatcttctttccgtttgtaaacttcattgttccaactctaagacacttgctgtcctaacacctgctataggctatccactgcatcatctctttttaagaaaaaaataggcccggacgggttagcaacaagcgactgccggtgccaacaacacaatccacaatctacagtcgacgcggccgccaccatgttcgtcttcctggtcctgctgcctctggtctcctcacagtgcgtcaatctgatcactcggactcagtcctatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctggacgtgtactatcacgagaacaataagagcagaatggagtccgagctgagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccgtgaacctgggccgcgacctgcctcagggcttcagcgccctggagcccctggtggatctgcctatcggcatcaacatcacccggtttcagacactgctggccctgcacagaagctacctgacacccggcgactcctctagcagctggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttcacgaggtgttcaacgcaacccgcttcgccagcgtgtacgcctggaataggaagcggatcagcaactgcgtggccgactatagcgtgctgtacaacctggcccccttcttcgcctttaagtgctatggcgtgtcccccacaaagctgaatgacctgtgctttaccaacgtctacgccgattctttcgtgatcaggggcaacgaggtgtcccagatcgcccccggccagacaggcaatatcgcagactacaattataagctgccagacgatttcaccggctgcgtgatcgcctggaacagcaacaagctggattccaaagtgtccggcaactacaattatctgtaccggctgtttagaaagagcaagctgaagcccttcgagagggacatctctacagaaatctaccaggccggcaataagccttgcaatggcgtggccggctttaactgttatttcccactccagtcctacggcttccgccccacatatggcgtgggccaccagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagtacgtgaacaattcctatgagtgcgacatcccaatcggcgccggcatctgtgcctcttaccagacccagacaaagtctcacagaagagcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaagagagccctgacaggcatcgccgtggagcaggataagaacacacaggaggtgttcgcccaggtgaagcaaatctacaagaccccccctatcaagtactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcacaacgcccaggccctgaataccctggtgaagcagctgagcagcaagttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgagctagcctcctgccatacttcctactcacatcatatctattttaaagaaaaaataggcccgaacactaatcgtgccggcagtgccactgcacacacaacactacacatacaatacactacaatggttgcagaagatgcccctgttaggggcacttgccgagtattatttcgaacaacaactttaatttttctatgcacactattagcattaagcatctctatcctttatgagagtttaataacccaaaagcaaatcatgagccaagcaggctcaactggatctaattctagattaggaagtatcactgatcttcttaataatattctctctgtcgcaaatcagattatatataactctgcagtcgctctacctctacaattggacactcttgaatcaacactccttacagccattaagtctcttcaaacaagtgacaagctagaacagaactgctcgtggggtgctgcactgattaatgataatagatacattaatggcatcaatcagttctatttctcaattgctgagggtcgcaagctgacacttggcccacttcttaatatacctagtttcattccaactgccacgacaccagagggctgcaccaggatcccatcattctcgctcaccaagacacactggtgttatacacacaatgttatcctgaatggatgccaggatcatgtatcctcaaatcaatttgtttccatgggaatcattgaacccacttctgccgggtttccatcctttcgaaccctaaagactctatatctcagcgatggggtcaatcgtaagagctgctctatcagtacagttccggggggttgtatgatgtactgtttcgtctctactcaaccagagagggatgactacttttctaccgctcctccagaacaacgaattattataatgtactataatgatacaatcgtggagcgcataattaatccacccggggtactagatgtatgggcaacattgaacccaggaacaggaagcggggtatattatttaggttgggtactctttccaatatatggcggcgtgattaaagatacgagtttatggaataatcaagcaaataaatactttatcccccagatggttgctgctctctgctcacaaaaccaggcaactcaagtccaaaatgctaagtcatcatactatagcagctggtttggcaatcgaatgattcagtctgggatcctggcatgtcctcttcaacaggatctaaccaatgagtgtttagttctgcccttttctaatgatcaggtgcttatgggtgctgaagggagattatacatgtatggtgactcggtgtattactaccaaagaagcaatagttggtggcctatgaccatgctgtataaggtaaccataacattcactaatggtcagccatctgctatatcagctcagaatgtgcccacacagcaggtccctagacctgggacaggaagctgctctgcaacaaatagatgtcccggtttttgcttgaaaggagtgtatgctgatgcctggttactgaccaacccttcgtctaccagtacatttggatcagaagcaaccttcactggttcttatctcaacgcagcaactcagcgtatcaatccgacgatgtatatcgcgaacaacacacagatcataagctcacagcaatttggatcaagcggtcaagaagcagcatatggccacacaacttgttttagggacacaggctctgttatggtatactgtatctatattattgaattgtcctcatctctcttaggacaatttcagattgtcccatttatccgtcaggtgacactatcctaaaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt

SEQ ID NO: 80 - DNA, 15192 bp, synthetic construct
  misc_feature 1..15192: note = Synthetic: nucleic acid sequence for CVL116
  source 1..15192: mol_type = other DNA
accagggggaaaacgaagtggtgactcaaatcatcgaagaccctcgagattacataggtccggaacctatggccttcgtgaccgacctcgagtcagagtagttcaataaggacctatcaagtttgggcaatttttcgtccccgacacaaaaatgtcatccgtgcttaaagcatatgagcgattcacactcactcaagaactgcaagatcagagtgaggaaggtacaatcccacctacaacactaaaaccggtaatcagggtatttatactaacctctaataacccagagctaagatcccggcttcttctattctgcctacggattgttctcagtaatggtgcaagggattcccatcgctttggagcattacttacaatgttttcgctaccatcagccacaatgctcaatcatgtcaaattagctgaccagtcaccagaagctgatatcgaaagggtagagatcgatggctttgaggagggatcattccgcttaatccccaatgctcgttcaggtatgagccgtggagagatcaatgcctatgctgcacttgcagaagatctacctgacacactaaaccatgcaacaccttttgttgattccgaagtcgagggaactgcatgggatgagattgagactttcttagatatgtgttacagtgtcctaatgcaggcatggatagtgacttgcaagtgcatgactgcgccagaccaacctgctgcttctattgagaaacgcctgcaaaaatatcgtcagcaaggcaggatcaacccgagatatctcctgcaaccggaggctcgaagaataatccagaatgtaatccgaaagggaatggtggtcagacatttcctcacctttgaactgcagcttgcccgagcacaaagccttgtatcaaataggtattatgctatggtaggggatgttggaaagtatatagagaattgtggaatgggaggcttctttttgacactaaaatatgcattaggaaccagatggcccacacttgctttagctgcattttcaggagagctaacaaagctaaagtccctcatggcattataccagacccttggtgagcaggcccgatatttggccctattggagtcaccacatttgatggattttgctgcagcaaactacccactgctatatagctatgctatgggaataggctatgtgttagatgtcaacatgaggaactacgctttctccagatcatacatgaataagacatatttccaattgggaatggaaactgcaagaaaacaacagggtgcagttgacatgaggatggcagaagatctcggtctaactcaagccgaacgcaccgagatggcaaatacacttgccaaattgaccacagcaaatcgaggggcagacaccaggggaggagtcaacccgttctcatctatcactgggacaactcaggtgcccgctgcagcaacaggtgacacattcgagagttacatggcagcggatcgactgaggcagagatatgctgatgcaggcacccacgatgatgagatgccaccattggaagaggaggaagaggacgacacatctgcaggtccacgcactggactaactcttgaacaagtggccttggacatccagaacgcagcagttggagctcccatccatacagatgacctgaatgccgcactgggtgatcttgacatctagacaattcagatcccaatcttaaatcgacacacctaattgaccagttagatggaactacagtggattccataaggttcctgcctaccatcggcttttaagaaaaaaataggcccggacgggttagcaacaagcgactgccgatgccaataacacaatccacaatctacaatggatcccactgatctgagcttctccccagatgagatcaataagctcatagagacaggcctgaatactgtggagtattttacttcccaacaagtcacaggaacatcctctcttggaaagaatacaataccaccaggggtcacaggactactaaccaatgctgcagaggcaaagatccaagagtcaaccaaccatcagaagggttcagttggtgggggcgcaaaaccaaagaaaccgcgaccaaaaattgccattgtgccagcagatgacaaaacggtgcccggaaagccgatcccaaaccctctattaggtctggactccaccccgagcacccaaactgtgcttgatctaagtgggaaaacattaccatcaggatcctataagggggttaaacttgcgaaatttggaaaagaaaatctgatgacacggttcatcgaggaacccagagagaatcctatcgcaaccaattcccccatcgattttaagaggggcagggataccggcgggttccatagaagggagtactcaatcggatgggtgggagatgaagtcaaggtcactgagtggtgcaatccatcctgttctccaatcaccgctgcagcaaggcgatttgaatgcacttgtcaccagtgtccagtcacttgctctgaatgtgaacgagatacttaatacagtgagaaatttggactctcggatgaatcaactggagacaaaagtagatcgcattctctcatctcagtctctaatccagaccatcaagaatgacatagttggacttaaagcagggatggctactttagaaggaatgattacaactgtgaaaatcatggacccgggagttcccagtaatgttactgtggaagatgtacgcaagaaactaagtaaccatgctgttgttgtgccagaatcattcaatgatagtttcttgactcaatctgaagatgtaatttcacttgatgagttggctcgaccaactgcaacaagtgttaagaagattgtcaggaaggttcctcctcagaaggatctgactggattgaagatcacactagagcaattggcaaaggattgcatcagcaaaccgaagatgagggaagagtatctcctcaaaatcaaccaggcttccagtgaggctcagctaattgacctcaagaaagcaatcatccgcagtgcaatttgatcaagaaacacccaattacactacactggtatgacactgtactaaccctgagggttttagaaaaaacgattaacgataaataagcccgaacactacacaccacccgaggcagccatgccatccatcagcatccccgcagaccccaccaatccacgtcaatcaataaaagcgttcccaattgtgatcaaccgtgatgggggtgagaaaggtcgcttggttaaacaactacgcacaacctacttgaatgacctagatactcatgagccactggtgacattcgtaaatacttatggattcatctacgaacaggatcggggaaataccattgtcggagaggatcaacatgggaagaaaagagaggctgtgactgctgcaatggttacccttggatgtgggcctaatctaccatcattagggaatgtcctgggacaactgagtgaattccaggtcattgttaggaagacatccagcaaagcggaagagatggtctttgaaattgttaagtatccgagaatatttcggggtcatacattaatccagaaaggactagtctgtgtctccgcagaaaaatttgttaagtcaccagggaaagtacaatctggaatggactatctcttcattccgacatttctgtcagtgacttactgtccagctgcaatcaaatttcaggtacctggccccatgttgaaaatgagatcaagatacactcagagcttacaacttgaactaatgataagaatcctgtgtaagcccgattcgccacttatgaaggtccatatccctgacaaggaaggaagaggatgtcttgtatcagtatggttgcatgtatgcaatatcttcaaatcaggaaacaagaatggcagtgagtggcaggaatactggatgagaaagtgtgctaacatgcaacttgaagtgtcgattgcagatatgtggggaccaactatcataattcatgccagaggtcacattcccaaaagtgctaagttgttttttggaaagggtggatggagctgccatccacttcacgaagttgttccaagtgtcactaaaacactatggtccgtgggctgtgagattacaaaggcgaaggcaataatacaagagagtagcatctctcttctcgtggagactactgacatcataagtccaaaagtcaaaatttcatctaagcatcgccgctttgggaaatcaaattggggtctgttcaagaaaaccaaatcactgcccaacctgacggagctggaatgagctagccctgctataggctatccactgcatcatctctttttaagaaaaaaataggcccggacgggttagcaacaagcgactgccggtgccaacaacacaatccacaatctacagtcgacgcggccgccaccatgttcgtgttcctggtgctgctgccactggtcagcagccagtgcgtgaatttcactacccggacccagctgcctcctgcttatacaaacagcttcaccaggggagtgtactaccccgataaggtgttccgcagctctgtgctgcacagcacccaggacttgtttcttcccttcttcagcaacgtgacctggttccacgccatccacgtgtccggcaccaacggcaccaaaagattcgccaatcctgtgctgcctttcaacgatggcgtgtactttgcctctacagagaagtctaacatcatcagaggctggatcttcggcacaaccctggattctaaaacccagagcctgctgatcgtgaacaatgccaccaatgtagtgatcaaggtttgtgagttccagttctgcaacgacccctttctgggcgtttactaccacaagaacaacaaaagctggatggaaagcgagttcagggtgtactctagcgccaacaactgtaccttcgagtacgtgagccagccttttctgatggacctggagggcaagcagggcaacttcaaaaacctgcgggagttcgtgtttaagaacatcgacggatacttcaagatttactccaagcacaccccaattaacttggttagaggactgccccaaggcttcagcgccctggaacccctggtggacctgcctatcggcatcaacatcacaagattccaaaccctgcatagaagctatctgacaccaggcgactctagctctggatggacagccggcgccgctgcttattacgtgggctacctgcagcctagaaccttcctcctgaaatacaacgagaacggaaccatcaccgacgccgtggactgcgccctggatcctctgagcgagacaaagtgcaccctgaagtccttcaccgtcgaaaagggcatctaccaaacaagcaacttcagagtgcagcctactgagagcatcgtgcggtttccaaacatcaccaacctgtgtccttttggcgaggtgttcaacgccacccggttcgcttccgtgtacgcctggaatagaaagcggatcagcaactgcgtggccgactacagcgtgctgtataatagcgcctcattttctacttttaaatgttacggcgtgtctcctacaaagctgaacgatctgtgcttcacaaatgtgtacgccgatagcttcgtgattagaggcgacgaggtgcggcagatcgcccctggccagaccggcaacatcgctgactacaactacaagctgcctgacgacttcaccggctgtgtgatcgcatggaacagcaataacctggactctaaggtgggcggaaactacaattacctgtacagactgttcagaaagagcaacctgaagcccttcgagagagatatctccacagaaatctatcaggccggatctacaccttgtaatggcgtgaagggcttcaactgctacttccctctgcaatcctacggctttcagcccacatacggcgtgggctaccagccataccgggtcgtggtgctgagcttcgagctgctgcacgcccctgccacagtgtgcggccctaagaagagcaccaatctggtaaagaacaagtgcgtcaacttcaatttcaacggcctgaccgggaccggagtgctgaccgaaagcaacaagaaattcctgccgttccagcagttcggcagagatatcgccgacaccaccgatgccgtgcgggacccccagacacttgaaatcctggacatcaccccttgcagttttggcggcgtgtccgtgatcacacctggaacaaacaccagcaaccaggtggccgtgctgtaccagggcgtcaactgcaccgaggtccccgtggccatccacgccgatcagctgacacctacatggcgggtgtactctaccggcagcaacgtgttccaaaccagagccggctgcctgatcggcgctgagcacgtgaacaacagctacgagtgcgacattcctatcggtgctggcatctgcgcctcttaccagacacagacaaacagccccagaagagcgagaagcgtggctagtcagagcatcatcgcctacaccatgagcctgggcgtggagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagttattgaccattgtcgttgctaatcgaaatagaatggagaattttgtttatcataaatgacgtacgaggcagaagcctccaggtctgacccagccaatcaaagcattataccagaccatggaatgcataccaaacattattgacactaatgacacacaaaattggttttaagaaaaaccaagagaacaataggccagaatggctgggtctcgggagatattactccctgaagtccatctcaattcaccaattgtaaagcataagctatactattacattctacttggaaacctcccaaatgagatcgacattgacgatttaggtccattacataatcaaaattggaatcaaatagcacatgaagagtctaacttagcccaacgcttggtaaatgtaagaaattttctaattacccacatccctgatcttagaaagggccattggcaagagtatgtcaatgtaatactgtggccgcgaattcttcccttgatcccggattttaaaatcaatgaccaattgcctctactcaaaaattgggacaagttagttaaagaatcatgttcagtaatcaatgcgggtacttcccagtgcattcagaatctcagctatggactgacaggtcgtgggaacctctttacacgatcacgtgaactctctggtgaccgcagggatattgatcttaagacggttgtggcagcatggcatgactcagactggaaaagaataagtgatttttggattatgatcaaattccagatgagacaattaattgttaggcaaacagatcataatgatcctgatttaatcacgtatatcgaaaatagagaaggcataatcatcataacccctgaactggtagcattatttaacactgagaatcatacactaacatacatgacctttgaaattgtactgatggtttcagatatgtacgaaggtcgtcacaacattttatcactatgcacagttagcacttacctgaatcctctgaagaaaagaataacatatttattgagccttgtagataacttagcttttcagataggtgatgctgtatataacataattgctttgctagaatcctttgtatatgcacagttgcaaatgtcagatcccatcccagaactcagaggacaattccatgcattcgtatgttctgagattcttgatgcactaaggggaactaatagtttcacccaggatgaatcaagaactgtgacaaccaatttgatatccccattccaagatctgaccccagatcttacggctgaattgctctgtataatgaggctttggggacaccccatgctcaccgccagtcaagctgcgggaaaggtacgcgagtccatgtgtgctggaaaagtattagactttcccaccattatgaaaacactagcctttttccatactattctgatcaatggatacaggaggaagcatcatggagtatggccacccttaaacttaccgggtaatgcttcaaagggtctcacggaacttatgaatgacaatactgagataagctatgaattcacacttaagcattggaaggaaatctctcttataaaattcaagaaatgttttgatgcagacgcaggtgaggaactcagtatatttatgaaagataaagcaattagtgccccaaaacaagattggatgagtgtgtttagaagaagcctaatcaaacagcgccatcagcatcatcaggtccccctaccaaatccattcaatcgacggctattgctaaactttctcggagatgacaaattcgacccgaatgtggagctacagtatgtaacatcaggtgagtatctacatgatgacacgttttgtgcatcatattcactaaaagagaaggaaattaaacctgatggtcgaatttttgcaaagttgactaagagaatgagatcatgtcaagttatagcagaatctcttttagcgaaccatgctgggaagttaatgaaagagaatggtgttgtgatgaatcagctatcattaacaaaatcactattaacaatgagtcagattggaataatatccgagaaagctagaaaatcgactcgagataacataaatcaacctggtttccagaatatccagagaaataaatcacatcactccaagcaagtcaatcagcgagatccaagtgatgactttgaattggcagcatcttttttaactactgatctcaaaaaatattgtttacaatggaggtaccagacaattatcccatttgctcaatcattaaacagaatgtatggttatcctcatctctttgagtggattcacttacggctaatgcgtagtacactttacgtgggggatcccttcaacccaccagcagataccagtcaatttgatctagataaagtaattaatggagatatcttcattgtatcacccagaggtggaattgaagggctatgtcaaaaggcttggacaatgatatctatctctgtgataattctatctgccacagagtctggcacacgagtaatgagtatggtgcagggagataatcaagcaattgctgtcaccacacgagtaccaaggagcctgccgactcttgagaaaaagactattgcttttagatcttgtaatctattctttgagaggttaaaatgtaataattttggattaggtcaccatttgaaagaacaagagactatcattagttctcacttctttgtttatagcaagagaatattctatcaggggaggattctaacgcaagccttaaaaaatgctagtaagctctgcttgacagctgatgtcctaggagaatgtacccaatcatcatgttctaatcttgcaactactgtcatgaggttaactgagaatggtgttgaaaaagatatctgtttctacttgaatatctatatgaccatcaaacagctctcctatgatatcatcttccctcaagtgtcaattcctggagatcagatcacattagaatacataaataatccacacctggtatcacgattggctcttctgccatcccagctaggaggtctaaactacctgtcatgcagtaggctgttcaatcgaaacataggcgacccggtggtttccgcagttgcagatcttaagagattaattaaatcaggatgtatggattactggatcctttataacttattagggagaaaaccgggaaacggctcatgggctactttagcagctgacccgtactcaatcaatatagagtatcaataccccccaactacagctcttaagaggcacacccaacaagctctgatggaactcagtacgaatccaatgttacgtggcatattctctgacaatgcacaggcagaagaaaataatcttgctagatttctcctggatagggaggtgatctttccgcgtgtagctcacatcatcattgagcaaaccagtgtcgggaggagaaaacagattcaaggatatttggattcaactagatcgataatgagtaaatcactagaaattaagcccttgtccaataggaagcttaatgaaatactggattacaacatcaattacctagcttacaatttggcattactcaagaatgctattgaacctccgacttatttgaaagcaatgactcttgaaacatgtagcatcgacattgcaaggagcctccggaagctctcctgggccccactcttgggtgggagaaatcttgaaggattagagacgccagatcccattgaaattactgcaggagcattaattgttggatcgggctactgtgaacagtgtgctgcaggagacaatcgattcacatggtttttcttgccatctggtatcgagataggaggggatccccgtgataatcctcctatccgtgtaccgtacattggctccaggactgatgagaggagggtagcctcaatggcatacatcaggggtgcctcgagtagcctaaaagcagttcttagactggcgggagtgtacatctgggcattcggagatactctggagaattggatagatgcactggatttgtctcacactagagttaacatcacacttgaacagctgcaatccctcaccccacttccaacctctgccaatctaacccatcggttggatgatggcacaactaccctaaagtttactcctgcgagctcttacaccttttcaagtttcactcatatatcaaatgatgagcaatacctgacaattaatgacaaaactgcagattcaaatataatctaccaacagttaatgatcactggactcggaattttagaaacatggaataatcccccaatcaatagaacattcgaagaatctaccctacatttgcacactggtgcatcatgttgtgtccgacctgtggactcctgcatcatctcagaagcattaacagtcaagccacatattacagtaccgtacagcaataaatttgtatttgatgaagacccgctatctgaatatgagactgcaaaactggaatcgttatcatttcaagcccaattaggcaacattgatgctgtagatatgacaggtaaattaacattattgtcccaattcactgcaaggcagattattaatgcaatcactggactcgatgagtctgtctctcttactaatgatgccattgttgcatcagactatgtctccaattggattagtgaatgcatgtataccaaattagatgaattatttatgtattgtgggtgggaactactattggaactatcctatcaaatgtattatctgagggtagttgggtggagtaatatagtggattattcttacatgatcttgagaagaatcccgggtgcagcattaaacaatctggcatctacattaagtcatccaaaacttttccgacgagctatcaacctagatatagttgcccccttaaatgctcctcattttgcatctctggactacatcaagatgagtgtggatgcaatactctggggctgtaaaagagtcatcaatgtgctctccaatggaggggacttagaattagttgtgacatctgaagatagccttattctcagtgaccgatccatgaatctcattgcaaggaaattaactttattatcactgattcaccataatggtttggaactaccaaagattaaggggttctctcctgatgagaagtgtttcgctttgacagaatttttgaggaaagtggtgaactcagggttgagttcaatagagaacctatcaaattttatgtacaatgtggagaacccacggcttgcagcattcgccagcaacaattactacctgaccagaaaattattgaattcaatacgagatactgagtcgggtcaagtagcagtcacctcatattatgaatcattagaatatattgatagtcttaagctaaccccacatgtgcctggtacctcatgcattgaggatgatagtctatgtacaaatgattacataatctggatcatagagtctaatgcaaacttggagaagtatccaattccaaatagccctgaggatgattccaatttccataactttaagttgaatgctccatcgcaccataccttacgcccattagggttgtcatcaactgcttggtataagggtataagctgttgcaggtaccttgagcgattaaagctaccacaaggtgatcatttatatattgcagaaggtagtggtgccagtatgacaatcatagaatacctattcccaggaagaaagatatattacaattctttatttagtagtggtgacaatcccccacaaagaaattatgcaccaatgcctactcagttcattgagagtgtcccatacaagctctggcaagcacacacagatcaatatcccgagatttttgaggacttcatccctctatggaacggaaatgccgccatgactgacataggaatgacagcttgtgtagaattcatcatcaatcgagtcggcccaaggacttgcagtttagtacatgtagatttggaatcaagtgcaagcttaaatcaacaatgcctgtcaaagccgataattaatgctatcatcactgctacaactgttttgtgccctcatggggtgcttattctgaaatatagttggttgccatttactagatttagtactttgatcactttcttatggtgctactttgagagaatcactgttcttaggagcacatattctggtccagctaatcatgaggtttatttaatttgtatccttgccaacaactttgcattccagactgtctcgcaggcaacaggaatggcgatgactttaaccgatcaagggtttactttgatatcacctgaaagaataaatcagtattgggatggtcacttgaagcaagaacgtatcgtagcagaagcaattgataaggtggttctaggagaagatgctctattcaattcgagtgataatgaattaattctcaaatgtggagggacaccaaatgcacggaatcttatcgatatcgagccagtcgcaactttcatagaatttgaacaactgatctgcacaatgttgacaacccacttgaaggaaataattgatataacaaggtctggaacccaggattatgaaagtttattactcactccttacaatttaggtcttcttggtaaaatcagtacgatagtgagattattaacagaaaggattctaaatcatactatcaggaattggttgatcctcccaccttcgctccggatgatcgtgaagcaggacttggaattcggcatattcaggattacttccatcctcaattctgatcggttcctgaagctttctccaaataggaaatacttgattacacaattaactgcaggctacattaggaaattgattgagggggattgtaatatcgatctaaccagacctatccaaaaacaaatctggaaagcattaggttgtgtagtctattgtcacgatccagtagatcaaagggaatcaacagagtttattgatataaatattaatgaagaaatagaccgcgggatcgatggcgaggaaatctaaatatatcaagaatcagaattagtttaagaaaaaagaagaggattaatcttggttttccccttggt